SEQUENCE LISTING (8 sequences)

SEQ ID NO: 1 - DNA, 66657 bp, synthetic construct
  misc_feature 1..66657: note = Genomic Nucleic Acid Sequence of ECACC 17062004 Pa193
  source 1..66657: mol_type = other DNA
cgattgcacaatcctaatagaaaaaatctatcacggacgttacctatctttaaaattaataaaattaatagtaatttggtaatttggtaaactttagtatttgaaagcctcgcggcactaagcttgtacatatcccgtcaagtttccgattccagctagctcgcggcagggtcgccggaaacttccggacttccaatccatgggtcgcggcgacaccacggcggactaagcggcaaggaccaaaactcgacgaacggaaccggaaatttgggagcgcgatagaatcgctcagctggacatatttctaacattcgatttaacactcaatccaaacactcaccaccagcgtctcccgccaaccgccgattcgatcctcacccaccagcggatcgcccgtataacatcctataacaccacctaacactcattcatcatcaaacccacccagacctacagcccacccacaagcagcccatagacgcgatccctggccccatagtacaatcgcgccatactcagtgtcgcggcagagcaccaggggccatccgccaaccaagccaccacgacgactccagaatcgaactccagggacgcagcaccaaatgaccaaacactacagccccgacgatctagtcacgccacaggaattcgcagacccgcatttcgcggcgatcaaccaaaagcgattcgacctgtacatcgacctgcgcgtccaaggttacagctcctggcgggtcttccgggccatctggggcgaggagcatatggacggtcccgcgcaggcccgcatcttcgcgatggagtccaacccgtactatcgcaagcagttcaaggccaagttgaacgcgaccaaaacgtccgacttgtggaacccaaagacggcgctccacgaactcctccaaatggttcgtgaccccaccgtcaaggactccagccgtctgtcggccatcaaggagttgaacgttctggctgaaatcacgttcgtggacgagtctggcaagaccaggatcggtcgcggtttggccgacttctacgcatcagaagccgaggctcagaccgcgaccgtcgctgctgcggccgaagccaatggctatgtgccggaaggcgaagagggcgatttcccgtccccgacgccggaaccgaccgaggaagaccgcgccaaccccatttagacataaaataacatcgttctaggcccgaatcggaccgaactaaggcgaaggtagcggtaagggacgaaaaacgattctagggcggttctaggaagttgaagcctaaccctcagaacgacaaagccccggactctagttcagaatccggggctttctttttgggcgccttactcgccagcttcgatgatttcgaagttgtatttgacgccttcgtgctcgaaagtcatcttgccagccgccttcagctgcatgcggaagcggatgtgtttcgaagaaggcaggccgaactcgatgaatgctgcgttggtggaccggaactcgccgcgcttgccttttacagtcacagccacgccatggcgctgggtgcgtttcttggaaacttctgggtctttccaggagttggcgatggctgccgacaaatccttggcctctttggctttctccggcgcgttcttggcctcttcgcgcatcttccggatttcttccagcgcttcctcttcggtgacttcttcttccggcttcagattctcttcctcggccttctcttcttccttcttcttggaagtgcgggtcttgtaaaccttggccggggcttcctcttcctggaaggcctcttcctcggccggcagagcattcaggatggcgaggcagcgacgctcagcagtcttgcggtcagagaagcgcttgacagtcgcatcagcgttgtgagcgttgtagaaagcgaccagttctttcatttctgcgttctggatgtcgccgaaggttttgatggagttggtcatcttgcgatcctctgttttggaagatttcttttgggcttcggtttgtcgcccccgttgaaagagattatgcctaggtcgatgctgcgtgtctacatctatttcatcagattgatcacaaagccgacgaacggttgtcggatgtgaaaacaccgcagagcaggctgcggtgtttgttggtgagggcgatggtcagaaagtcgggatcgtgatcggctcgatgggcggatcgccgggcctgtcgttgtccgatgcgggcgccgggttggacgggattgcgccggaccgaagctccagagattccgactgcctgctcacctgcggagctggaggcgcctttggcgcaggaggagcaggcgggaccggcaggcctggaggcgccatctgcgtcatgggcggttccggaagatcgggaaggcttggcgctatgctgccaaaggaccgctgcgactgctgtgccggattttcgccgaacatttcggcggatcggttgtccttgatgacttgacctatggccgtcttgtcggcgcccggttctgccggatcgaagccagtgaatggagtggttgtgcagcgcatgttcggatgggtcggcagattgttcagggcgatggagtccgcccagacgtacgggtttttatgcttgccagggctgtggctgatgatctcggcaggaatgtgaccgttcctgttcagccatctcgccatctcctgccgattcagagggctggccacgatctggccagtggaatcttcgacgatagcgaacagatcaccgagtttgaatccgatgattgtgtagttggacctggactcgccgacgaaagccagggtttcgatgttgtggtagatatcttggtcttcccagatgtacagcatggttcactcctcaataaggctgcttgttgtgttcgaccagagacataccggccggaactttgaagcggatgtattcgatcatcatcgcggttcggcgtttgttggcgccggattcgtcgttgacgaagagttcgcattggtgcggaatgactcgcgtcaccaggtcttcgcctttcgaaccgtagaacttgatgtaggctgcgacgcctcgcgtcatgctcagatggatcgtttcgcagagtcgctgcgcggccgacgcgctggacagatcggtcggactgaccgtcgcccagtaatggccactttgttctttatcgggcataagctaccctccaatgagaaagcctctgccgaggctggttgttagtcgagccagtcagtcgtacttcctgccgtgaaatgcgtgtccgcagccgaagattgatctgtatgcgttgattgaattcctgttcctttcagattcgatttgaatactgagcatctccagctcgtatcgggccagtccgccccattcgcgctcaatgcgtgaatactcggcctccagttgcctcaatcgttcgctcatataacctccaacaatcgtcattaatcgatcaacccctgccgaaccgcagaggctggttgttaatcgcgcttcagcaaaaccactttctcgtttgcgcgatgattcctgtgccagtcttcccaatagtctccgcactgacgggccaactccagagtgacatagccgtcgtcgctgctacattcgacagaaaggacaatcgcgttgcctctggactcgctgccagacccgaatccgatcagaacttcgtcgccagttttcagctcatctgccctgattactttggccatggcgttctcctattgggtagcgccgcgagggcggatcgggtcgatgtcatgtaagcagatggtgggatagtccctggccagcttgagcttgaagtccacccagtcagcaccgatgagcgccgcgacttcgatgcttactttgcggtgcggcgcagctccagatcgcaggactatagcccgacgccgaacgggatcgttgacgtcaacatgcgtgagtatttcgtcgccgacgagcaactcgcaagccatcaatattttggccataatacactcctatgaggaggcgcgcccaggacgatcccagccgcgccgatggattaacgtttgtgaaggatggacactgcgtccacgtcgaggatgctgatggtacggcgacggcgcggattgctgcgttcatggatgcgaatcaggtcagcggatggcgcttcgtgcaccacccatacatcctccggcttctgcgtccggagcaggaccgtcacttctgtgttctgggcgaggccgttgcagatgaacgtgaacttggacgagggagttctgtacatttcgagattcctttttggacttcgggtccggctcctcagccggtgaagagattatgcctgatcatcgcagtcgagtaaagcacttgtgtaccaattctccagttaaatggaaccaaagcgcggtatcgctgattgctacgctcccacgccatggcccttcttgctcacagacttcgaaccacaagctgatttccattctggccaggactcggccgaaggattcgagccggcaccggctggacagaatgtcgacattcacccgtcgattgatttcgtaccggcgaccgttgatgacgaataccaagcgtagagtttgtccgtccagacattcccagtatctggtttcataccgaagccaatagcgcttgctagcagggcctaccatagtcatttaatcgatgctcctggccgcgccacgcggtccggtcggcgggacgggatcgaaccgacccaggatgtagtcgggccggcgcgcttcctgaggacacacggcgagaaggcgccatcctgcgtccagggcatgctggagttcgtccgtgcagcagtcttccttgagcagcagacgattgatgctctggagatttggaccagggatagcagagccggtgaccgagttccagccttcgataccgtgcaccatctgcggctggttgatgtagccttctgacctgccagccaaccggctcgcggccagctccaggcgctccagcattggccgcagagcggcttccgggtcaacatcgtcccagagcaggaccaggttgatgatggtgtacggatagtccttgtccagatcccaggccgaagcggtcagacggcccaggccgatttcattacacatcacgggaacgtcgttgctccaggtggacggctcccagttccgctcgccaggattcccgattgttacgccttccaggttccccaggagaacgtggagcttgctgacatattccgcctccaaggctttccgctcttcgtcggtctggttgtggcgatagaaggatggagggctgacttttgcatggtagagtttcataactgttcctcggttttggaaggcttgaacgttagagaattgtgtcgcagtatttctcgaaaggactctggcgcttcttctcgcagatcgcacaggtgatttccaggtcgggcagctcgctgtaagtcttgccgaggtacagccagcgcttgcacaggctgcgaccgtccgacgtgaagaaatggactttgcgagcgtcgccgggttgcgcccagccgccctgatagttttcgcgcttgctcatggcgatatttccttttggatcgtggatccatggtgtacgttcagtcacccaaactccatctacatagatcaggactgacagagcagtcgcgccaaacccgaagcctacatagaatgaacctggatcgatttccatggtggcgctattagtggcgctattgcaatcgacttcaaagtcgaatagcgaatgaccaatcataccatcaccatgtcgatttcattgacgaagaaatggacatcgacaccatcggcgctgccggtgtaacgcagccgagtcgtcccatcaatgtgggcctcctcgacgccgacgacttccaggatggtgtcgttgcagtacagtttgacgaacatcttgatgccagagccaatcgcctcgaaggcgatctgcttgtacaggtcttgcttgatcatgctttacgctcctgtttgcggatgtattctgccctggcttcggcttcgagcttcgcccaaagcgcgttgtcgactggaccccagggacctgtctgagttccgtccaccggtgcgaacttgccaccggcgcgacgatacttggacagagttctcagaccgatgattttctcttcttgggaatcgagttggacgatcacttaacatacttcccgaagtagcaggtgtggaggatttccaggcccttggaaatggccttcagcttgataccgaaattcttgatcgcccagtccagatcgtggtgatagaactcgatgatgttgacgatttgaccggcgctgtagtggatgacgcgaaccttgccattccagcggaaatcttcgcggctgtttacggcctcgacttcgatagtgtcatttctgccgatggtccatttggagctagtcatgtcgcaatcctctttttggagcgtttcgcgtttcgatgaggtaactatacttcagtcacctcatcgagtaaagcccttttgaacagattgcctaaaatttctggaagacaaaatcaatcggtcgcgtcatcgatagcgaacgccgagcgcgccaggtcgatgatctctggccaggtcaaagtgaaggttttgccgtcatcgagaatgatgatgggagtccgcagcggctcgccaaccagcagttcgtaggaatgtttctcgctggatgcggagccgatgcgggtgtggagggtgagttaggccggggaaatctcggccgccgtaaaggccgcaatgcgaccgctattcagcctgacgaacaccgggaatgcgccgccctcgcgaatctcgctgatcacgccgtggccgccgccttgcgcatagacattctgtccaacagacaattttttgaggatgtcgctaaaaactacggttttcatcgtcttgccctcggtgatgcccggcgaaccgggcggggtttgctacagcatgcctgctttgcgttcggccttgatggtgtataccgcctcatcgaccagccactcttcagcttccaggtaggctttggcgacttctacggttacgttggcgaatttggctacgttctggattttggcttgcttggtcatttcgatttccttttgtctgaagggtttcgcgtttcgatgaggtaactatacctcagtcacctcatcgagtaaagcattttcttcagattatctgaaaccttttgaagccaggaattgccgccagagccagtcgatgtgttcgttgcagtaacgctcgccatcggacgacatagctaaggcgccatcattgatgccgaagtgggccgcagcgagacattcgaactcgaactggagcgttcctgggcgagggatatgggacatggcgtatttctgcacggtcagtgatcctcagaaggtttcgcgtttgcgatagccgctactttctgcataccgaggcgatagaattcggcctcttccctggccttggccagattatccctggcttcgcacgccagcttgtactgattgttggcattctggatgcttccgtccagactcctgtccagttcttcccgaagagccttctgcgacgctagctcggcctccaaatcctggattctgatctgaagctggcgattcctttcagacatctcgttttcgcgggcgatggagttctgaaggcattcgtcaaggcgagtattttcgcgcaccagctgatgcttttcgactttcgcctttctgcacgtctctcgcattgctgaaagctcactcgcatgcagatcgtcatccgggtctttacgctcttccttcgactccatccgcatatagtactggccggctgcgaaggcgagcactgcctggtcggcatacgctgcggcatcggtcatgtatctggatttgtagcgaggcagaaacttgcactcctctggatcccagaggagactatcggcaataggccagaatttgcggaagccatcaggatcgacttctacattgaccatttgcagctggccggcctccacttcgacttccagaggaacctgaatggtgccggtgatgtactggaagtacactatgctagacataacacaatcctcataagaagttgcggccaggaccatcccagccgcatcgtaatcactcttcgccttcgtccgcgctcagccactcttcgaaggcgaaatttaccttcgactcgacccaatcctgcagctcatcggcgaactcgtcactgtcgatgtccatggtgattcccataacttccatgttccatagcgcggcattcagttcgaactgtaccatctgctcgccatcgacttccaacaggatgcggtctgcgacttcatagtcgtctacatcgaacagatggccgcccgccaggaggtgctggacgaaagcttcatcgaggttggtgacttcgatctggacttgtttggtcattttcgtatccttacttagcggctttgaattgggctttgaggttggcaagttcagctttcagatcgatcatcgcctgaccgcgcaggccttccatttcagcctcgctgattgcgatcttggctgcgcggattttgtcgttgatttgatctttagtcatttcggcttccttttgtttgaagggtttcgcgtttcgatgaggagattatgccgatagtcagaatggaagtaaagtgtctgagtgaagaaatttctgtacaccgacgaacggtcgcgctcgactgtctgctgcggtgtcgatatactcggcctattgcgaacgatggactgattaaatgttcaagctcaatcctgcactgcgagcggtctggcgaactcgtgcccgttacaaagtcatctatggcggccgggcgtcttcgaagtcgcacgacgctggcggtatagccgttttcctcgcggccaactacaagctcaaattcctctgtgctcgccagttccagaaccgcatcagcgaatctgtctacacattgatcaaagacaagatcgaaaactcagagtataatggcgaattcatcttcaccaagaactccatcaagcacaagattaccggctcagagttcctgttttatgggatcgcccgtaacctgtcggaaatcaagtccaccgaaggcatcgacattctctggcttgaggaagctcactacctgacccaggagcaatgggaagttatcgagcctaccatccggaaagaaaactctgaaatctggatcatcttcaaccctaacgaagtcaccgatttcgtgtatcagaacttcgtggtcaaacccccgaaagactcctgcgtcaagatgatcaactggaacgaaaatccgttcctcagtgagacgatgctcaaagtgattcacgaagcctatgagcgcgaccgggagcaggccgagcacatttatggcgggattccgaagacgggaggcgacaaatccgtcatcaacctcaagttcattctcgcggccatcgacgctcacaagaaactcggctgggagccggccggatcgaagcgcatcggcttcgacgttgcggacgacggcgaggatgcgaacgcaactacgctcatgcacggcaacgtcatcatggaagtggacgaatgggacggcctggaagatgaactgctcaagtcgtccagccgcgtttacaacctggcgaagatgaaaggcgcctcggtcacttatgactccatcggcgtcggtgctcacgtcggttcgaagttcgccgaattgaacgatgccagcccagacttcaaactgatctatgatccattcaacgcgggcggcgctgtcgataagcctgatgacgtctacatgaagctgccgcacacgacgatcaagaacaaagaccacttcagcaacattaaggcgcagaagtgggaagaagtcgcgacccgattccggaagacttatgaagcggttgagcatggaaaggttcatccattcgacgaattgatttcgatcaactctgaaacgattcacccggacaaactgaatcagctgtgcatcgaactttcgtcgccgcgcaaagacctggacatgaacggccggttcaaagtcgagtccaagaaggatatgcgcgagaagcgcaagatcaagtcgccgaacatcgctgactcggtgattatgtcggccattctgccgatccggaagcccaaaggcttcttcgacttctaaacatagaaaagcccggagcgatccgggcttcgggtcttactcggtgtggttcctggcgctgagtgtcgacgaaacggcctcgccgacgcccagagcttcctgaccggccgcgagcgcttcggcttccgactcgacgttgaagtcatcaccttggccattgcctggcggaacctcgaccagcacggcttcttcgccttcgaaacgcaggtcataggtcttgtcgacggacagaccgtaacgagcgttgagcgcgtcccagagctgggcctcataggtacgcaggtcttgcagagctttctggtggctgagcatggccatgtcaacggcccgttgcagggtttcgtccaggacgttgaaccgaatgcgaagagcacgaatccgctcgacggtttccgcatccactacatgtctttcgatcattgcttttcacctttgctgaatgttacgttgtagccgttgtcggccaggtaggtcagggcgccttcgaaggaagttccgacgaggtgcttgagcttcatttcgcgttgcgcggccaaccagaatgcagttccggaaaactctgcgcggccttcggacaaaaccctgccatcaggcccggcgatccgtacatggacggaagatagcttgatcggcatcagtgaatacctccactggcttgcgacggcatgctttcggcgcgagcggcttcgcagtccgggcagggacaggcttggcggacgcgctccaactcgtcggcatccatgacatagagcttcccatcggcagtgtcatgtgccatagcgatgttcgggaagtcggttgcgctcaggccggcgacagtgcggatttcgtcccagagcaccagatgctcggcattcagtcgggcggcgagcgcttcgtgctctttagcgacgcgcgccatgaactcgtccatccggaatgcgaattccgcatcgatggcacgggccgaggccatggagctgagtcgaatcggttctttcttcatgatgattctcttttggttggtggttgttagctgctcaggcctattacagcctgagcagcatgatgaagatgaacgcaaggaacgcgaacggcacaagtatgccggacatcagctttgacaggcgctgcatctcagcgtatttctcggtagacattccgtgctgcgccgcctcggccgcgaagcgggctttcgcctcgataacttccgggcgcagcgacaggacatattctaaggcctcttcccgcgctttttcggcctcgaccgacctagggtcgcgggccgagacttcgctgtgccctggcctcgcgggatgggcttgcagcgatggagggagttcagcggccacgactccataatcggcacaggcccaagcgatcccaatgaggatcgcgaggatggactggacgattcgcagcatcactttgtcgctagggaagctcatgatcaatcctccaccgaccgaacgatttccatgttgcggccggcattggtcccggcattgaaggcgcgccgaccgtcgctatcgtccagacgcatgagcttagtaacattagacttcttgtagccaggatcaccgaaatgttcgtgaaccgcagcttccttcgccaccaccagagacgttccggcagaagataccagctccatgcgcttcctggtgatggattgaaggcgatagctgatttcctgggtcgcggccagcttgaattgcgcggcaacctttacgttgaaacgttcgtacccttgagacttctgatactcccggcacagacgatcaacggcctcgaccaaggagttgaacatgttcactgccagctcaacgtccgacttgtagcctttaaagcgtacggcatgaccccagcgcttggtagtgcttccgtcgcgggcactcctggacgccttagccgatgctctgtggttgttgacgccaccggcgaaatccatgatgcaatcattgtacgtcgccacggccaccgagaagaacttcatccagttcgggattgcggaatagtaacgagtggcaatttgctcatcgaactcttcgcgaatctcgccggtcgcttcgaagtcgtgaaggtcatatttgtccttcaatttcttcacgcgctcggctgcgatagcagcttcgtgcggactggaggagtcggccgccatggcggtcagtttgcggatgcggtctttcgccttctcgatggcttccggagtgaattcgttctggtcggtcatggtcggttccttttgtctgaagggtttcgcgtttcaatggagctattctgccttcattcagaatggaagtaaagcactttcttccactatttcggcatcgactggaagaaattccagatccaatcacctgctaccagcaagaggatgagggcgacaaagaacaggacggcggccgcgagctgtgcgcccggcttcagcttgggatggctgagcttgggctctgcgggaacggatggagcgttggcgctgcgaccgtcttctcccaggccgtagccgatgccgcgctcgcgcgcatggagcgacaccagaagtttcagatactcggtctgcttttgcgactcttcgtagatgccggcgacggcgaaccagagggcgaacacgacggacatacacacaacccaggcgccggtcagcaggagggccagcgggccgatgacgaagacggtggcagccaggacgattgcgccgccccagataatgaagccggccagaccattggtgatgtcgatacagaactttttcatttgcggattccttcggttacaggattgaggggatttggaactctgcgccaccgagcacatcggtgacgacttcccagagcgtcggcactgaccactggtagcggtcgaagtcggtttcaggatcgacgcccagcgtcacataggaagtggcaggccatttatgaacgctacctttccgcatgagagtcgccacgcggccgtcaccgagcgggttcgtcttgtgcggaacgtacgcagacatcgaatattgctgcccagcatcaacgatgaatctttgtctctggcgcagacatacagagccaacaggattccaaccgcgaccgccgcaggcctggcgagccgaagaatgctcatacccttcatgagtcatttcgccgaggcaagccgagcgatcttcgtaataattgatgttctccatcgatccaaccagcaccagagactcgaaatcgaatcggtgatcgtgaatggacgagtgatggacgcaacgccggcgcggcaactccgggtgccaaacatggaggcgccctgccgggagctggacctggatgaaccccaggccgtgcaaagtgatcttgtccttcagcggatcatggactgtactcatggataatcctcaatagcagaagtgtatggtgagagttacgatggccaagccggtcgcccatagaagggcgaaccaggccatagcttttatggttatgtagatcatccgaagaattttccggcacagatcgggccgattcccatttcgatggacgcgtggttggtcaactcgcgaccgcagcaggagcattgaccagtcttccggccgtaggcgactgccgattccatcggcttttcgaacatcttcagaacgtcgtcgtgctcggtatcggtgcagtcgcgactcttgatgaatttgccattggtgatacggccgaggtagatgtcgcccaggacgtacaagctaccggcgttccggctgtgagcgctagcctctttcaccacaacgatgagcggctcctcgccttcgccagccaggcgaattttcgggcgcttgattccagattctttcgccttctcaaacgccttctcgatgccggaaatgtccagagtcggcgcagcagcttcctgcgcggccactttctcgcgatacttggcgaagttttcgatggcgcgcttcgcagcagcgatctgattttctgtcaaggagccgtatttatacaacgactcctgaaggctctgagcgaagctgaaggaatttccagtccaccactcgacgatgtccgggtgcgcggcttcgaaggccttaattttgaggtcgcgctcttgcgcagcgctacagattttctcgatgcgcttttctgccgccttagcacggctcttggcgcgctgctccgggctggttttgtactctttgtatccgacaccgccgcaggcgaaacaggcgcgaccataagatgaagggccacggtacaggccggtgcctgcgcattttgtgcacttgtcgcgatacagcttcacttccttccgggagttcgggcgggcgcccatggacacgtcttccagcgtcttcggcgcttcgtcagtgatcgctacggtagcgaagtcatcgcccaggtcttcgaagccggtgaacagattctctgctgcgttcatatcgattctcctgtttggaaagttcgtttcgatgagttgactatacgccagaaatggaaaaacggtagcgatttctcactaccgttcgtcgggtcgaagataatcaatagatgtcgctattgatcttgaacccatgctcagcgccgtcgttgtagtcatactcagcatagctgtcgcagtagtcgttcaaatgctgaatgatgcctagaacgtcgttagcggccttgtgccgtttcgcggcgatggtcttggcgatgctatcgccgacttccagggtttcggccgtccggcgatggatcagtagacgactccaaagatagagccggacgcgacgaattatgtcgcgatggcgctccagtcgtccgtgcagctcttcaatttcttgctcgcgagatttcaccactcgtcgaagctgttgaacttccaattccaaatcggccttagtagccatgttcacctcagaaaggaaaatcgtctgggactccaggaagctcgacgattattgttgagcctgatcggtccagaatgcatccgaccgagccggccctgtggggatagggtctacacgaactataacaggtgacttccaggactatccgccaggtcgatcctccacaccatctacagactggtgggcgcttcgacttcagttttcgttcgtccaagacggcgcgtctgtcgcaggaaaggcagcgcgccttaaccgacgtaaccatgggtatagtcgatcattcggatgagatcatcatcctctgattccctcgacttcagaccgcccaaagagtcagactggccgttgcatttcgcatcgatatccatcgaagacaagaatcctgccgatactacggtgatatcaactttgtcgccggccttcttgaagccttcgcactcagcatcgagggccacaaccgcactcgcagccatgttcacatgacttatgaggtcggggaagatcacgggaacttcacgcgacatgccacggaccgtcatcttcaggactacatacttcatactcactatcccttttgtatgtgaggaaagaatttactgttttccggatggtggaagcgctcggccgcaggcggtcttccttccggacattgaatcgtcgaaggcgggaaaaccgcgcaggcgataatcgccgcgaggagtgcagcggatgtcgacatccagagggcggtttccaggctcacccgaacttccggacggcgcggcttcattcgctccaccctcttcccggttcataggatggtagactgccgccgcgaacccagttgtcccacatgcggttcagaccagtcggtggctggggcggcggactgtagaatccgggagtctcagtgctgctcaggaaataaggcgtgcaaacggcctggacggccagttggtgccgctcccacatcttatcaatcgctcttagcatgacgtcttcgcactgagccttactgtcgaaccgtctgctagtgtggtccggtatctggatgcatccgtctccagtgcaaaggaaagcagtagcaatccacacggtgatactcgccatttcgtcaccctctttagttgatgagcagagtctattccatctgctcgccaggagtaaagcgcttttcgtcgggataaatgccgatgatgtctgcgtcgagcatccaaatgtccacggacggatcgtcgctgctgatttgatataggttgtacagctcccgtccgccgacagcacgctcacctcgcggctcaaccgccagcaccttgccgtgtccttctccgtgctcgtccaggtatatgacgtgatcaccgacttcatagctttcttttgtgacgaggcgtgaacgcgagctgttctgcgattctactacccaggaatccagcacgccgcctttcacgtcgcgaaacaatcttgccgatttgtcgcaatcgaatacgcccagaacttcgccgtctttcaacacgatatgtacgattggcaaaagagtattcatgtttaatctccattggttgataattagagtctaatctgccgaaaagttcccgtaaagaattattttctcataactgattagttgcaactgttaatctgatgtatatgtttgaatctcttttgaacgtttgatgtttcccctataataagcgcacacagccaacaaccacgtggaactacaatgtttaaactttcctggatattcgggcgcaaaaaggataatgctgcctgttctgaatcggcgccggagaaagtcgcacaaatccctcaacacgatccgctcgaccccatgatcaagctgggaaggattcgcggctggaacgtcgagccggagaaagccccggtaattcgtagtgtgaaagatttcctggagccgggcctatctgtagcaatggatagtgcgtatggtgatggaccaactccggccgcgaaggctgctgcgggcggccagaatccctatgtcgtcccgactatgttgcaggactggtacaattcccaagggttcatcggataccaagcttgcgcaatcatttcccaacactggttggtggacaaagcttgttccatgtctggggaagacgcagcacggaacggatgggaactcaaatcggacggcaggaagctatccgatgaacaaagcgcgctgatcgcccggcgcgacatggagtttcgcgtcaaagacaacctcgtcgaactcaacagattcaagaacgttttcggcgttcgcatcgcgctgttcgttgtggagtctgacgatccggactactacgaaaaaccgttcaatccggatggaatcacgcccggctcctacaagggaatctcccagatcgatccatactgggcgatgccgcagctcactgccggctcgacggccgatccgtcttccgaacatttctatgagccggatttctggattatcagcgggaagaaatatcatcgcagccatctagtagtcgttcgcggaccgcagccgccagatatcctgaagcctacatacatcttcggcggtatcccgctcacccagagaatctatgagcgcgtgtatgcggcggaacggacggcgaacgaagccccgctgcttgccatgtcgaagcgaaccagcaccattcacgttgacgtggaaaaggccatcgcgaatgaagacgctttcaacgctcgcctggcgttctggattgccaatcgcgataaccacggcgtgaaagtcttgggtactgatgaaagcatggagcagttcgacacgaaccttgccgacttcgacagcatcatcatgaaccaatatcagctggtcgctgccatcgccaagactccagccacgaagctcctcggcacttctccaaaaggattcaatgccactggtgagcacgaaacgatttcttatcacgaagaactggagtccattcaagagcacatattcgacccacttcttgaacgccactatttgcttctggcgaagtcggaagaaatcgatgtgcagctggaaatcgtctggaaccctgtggactccacgtccagccagcaacaagccgaattgaacaataagaaagccgcgaccgatgaaatctacatcaactctggcgttgtgtcgccggatgaagttcgcgagcgtctgcgcgacgatccgcgttccggctacaaccggctcaccgacgatcaggcagaaaccgaaccgggcatgtctccggaaaacctggccgaattcgagaaggccggtgcacagtcggccaaggcgaaaggcgaagccgagcgagccgaagcccaggcgggcgccgtagagggcgcaggcgacccggttcccgccgctccacgcgggactaagcctctcgcgaaagcggccgaggaaggggccagcgaggccgctgaaccgccgtcgcggccggaccccaaggccgaactgcggaacttgttggtcgatcttttgtcgaagctccaagacctggacgacattaaggcgccggacggcgtagacatagagcacaatgatgcgcctggcgtaaagcgcacgtccaaacctggcgtgtctggcatggagccttcggtgttttcgtccaaccgcatcgtcgggcctcgtgatcattcggaactccagagaatcaaggtgaatggaataaccaccttgatcgaaaatccgcgcggaagcattcggcaaggaaaggatgggagttggcgagttcagatgaaacaccactacgggttcatcaagggaacgaaaggcgctgatggggatgaagtcgattgcttcgtcggtccgaatctgggatccaaacgggtcttcgtcgtcaaccaggtgaacaaggaaggtcagtttgacgagcacaagtgcatgctcggcttcaacaacatcaacgatgcgaagtctggatatctgtcctgcttccgcccaggttgggatggtctcggctccatccatgaagttgacctacccgctttccgtcgctggctggcgaacggcgacacaaccaaaccgtttggaggcgagtgatggcattcaaggcctccaaaaagcgcgaacgccgggcacctcttccggtcggaagagggaagcccataattccatcgtcaggaatcgaggcctggtatcgaaagcagatgaaggatatttccaagctcatgatcgccgactatcgaagcgagattgagaaggcgctgtcccagcctgcggccgaacggttcttcgccagcgacgaatccgttaacgtcctgttcaagatgacccttcgaagcctacagcagcgatggaaccgcatttttgaaggcttcgcggccaagatcgccccggagtttgtcaaccggaccgaagaagcggccaccgccgcgaccctgcacagcttgtctgtggccggcgtcgatcagccaagagctgcgtacaatgagagcgtcaggaataccctggaggcagcaactacttacaaccatactctcatcaccaggattcaagaggaagtccacgagaagatttatacatcagtaatgttgtctctgacttccccaaatccggaagagcaaggaacttccggcataacgaacgcacttcgcaaagtcgggaagttctctgaagatcgaatcgaactcatcgcaagagatcaaaccagcaagctttacagttctctgagcgatgagagaatggcggaaaatggagtcgaggaattcgagtggatgcactcttctgccggcaagactcctcgccatacccacctggagaaagatgggaaaaggttcaagctgaatgaccccagactttgggagggtccgaaggccgaccaggggccgccaggatgggcgattaactgtaggtgcagaaagataccaatcatttgatatcgataggagcgctatatgccgttagttcatggcacttccaatgaggctcgttctgaaaacatcaagcgggagatcgaagccggtaaagacccaaagcaggctgcggccatcgcttactccatccagcgcagtgagaaagggaagacggcgaaagattgttcgcctgagctcgttgccgatcttcgcgccctggtggacgctctgtcgaggctcgtgaaatgaaccgcaagacatgccggcgccgactcgtggtcgatgtaatcagggccaatattcacggcggattcttcagcctgaagtttgccgccatcgatttggcaatcatcggcgtcgccatcttgatgacttttggccgataatgctgagaaaatctggattctgactaaaattctagtccggatagccgcaagttaccgtttacggaaaatagcagtaatttggaaagcctactgccgcgaggctttaacagagctagttcctaatttccgatttagccgcgtgcttcaaaagtatatagcctgggaaattagaagtaacgtcccaatagaattcatctataagtaacgttataatataacgtcaatctatatgctctagacgtattgaaatttaatttttaatcggtaaattggtaatttgaattagtttatgagttgaaagcctcgcggcagtaggcttagacaaatcccgtcaagttccctagaccaaattaccggattttcgcggctgaggaaactggtaattagatcataatacaaattatagtgtaagttaacagtcgcgactacatctaattattgttccgcttatttacccttagatgtactgcgtatataatacagccatagtccacgactcttcgaattaacgatggcaaagtcgaaaagaaaaattgacgaaaatggatatatgaccatcgagggttgcccgatcagctcttatggcgttttccaatattctgctggtcaactcggtcttccgggcgatccgatgcggattgtcaacgtatatcgcccggagtctgccgtcagcgatcctgagtacatcgaatctctgaagaatctcccgttgatcgacgagcatgagatgctgtcgggattcgacgacgatgacgacagtgtggcccccgaagacaaaggggtggagggcatcatcacctccaacgcttactacgaagccccatgggcgcgcggcgatatccgcatctattcccgcaacatgcagaatcagctggaaaggggcaaagaagacctgtccctaggctatagttgccgctacactgagcaacccggcatctggaacggaacgccttatgaagtcgtccaggacaagatgcgcggcaaccacatcgccctggtaaaagaggggcgtgtgccgggggccagagtattggatggtctgtgttttgaccatctcagttttgatttcagaccatccgatgagggtaatgaaatgagtctcaagaaagccaagcagaagccccctgtccagcgcgtagggcaagctgctgactcggcggtcgaagagttgcgcgccctgtggccgaagctctctgcgtctgtccagaagttcctgggcgaagaggcgcaggagccggagcatcaggaaggcgcggccgctccggccgaaccgaccgacagcgagcacatgaccgagcatccgactctggaaggcgctcagaaggacaacgaagagcatgaagaggcgccttccgttgtcgatccggctgtggccgccgccgagtcggagcgtcaagaaagtgccgcatccgaaatgtccggtgaaggcgaagtcgccgaactgatctcccaggtcaaggccattctggctcgactggagggcacggcggccgaaggggcggacgaagagcatggcgaaggcaaagatgtcgtcgagggtttggaggagcagagcagcctcagcggctcgcaaaccgccagcgacgatggtggtgagggcaaggataacagcgaggaacttcctgaaatggcacagaagaacgcgcaagatgctgcaattcgcggtctctatcgcgacattgctgctaaagatcgcctctacaagcgtcttagctccgtggttggcgcgttcgatcaccgagctatggactcggctgaagtcgctgtttacggcgtgaagaagctgaacatcaactgcgcgaagggccaggaagctctggcactcgacatgtacctgaaaggcgtcgaggcctcacgtggcgcggccagccgtcaatcgaaagcccaggattcggccggctctgctccgcagtgcgccgagctggacagctacctgaagggggagtaactcatgttccagaaacaagtttaccgccagtacactcctggttttccgggcgatctgatcgaggacggcccgaagcgggcgcggccgggtcgaatcatgtctctgtctgccgtaaatccggctgccaccgccaccggccccaaccgcatcagtcgcgctttcggttacgccggtgacgtcagcgccctcggcgaaggtcagccgaagaccatcgcggctcgtgcttctgaagtcgtgatcggcggcgccaacttctttggtgtcctcggtcatccgaagcactatgccctgttcggttcggccggagactccctggctcccagctatgatctgcccgatggcgccgaaggcgagttcttcgacatggccaccggcctggtcgtcgaaattttcaacggcgccgcaaccgccctggacctggactatggtgacctggtcgcctatgtacccaacaacctgcctaccgccgacaacgcgcttggcctgccggcaggcgccctggtgggcttcaaggctggctccatgccgaccggtttggtccagattcccaacgcacgcatcgttaacgccatcagtctgccggcccagtcggcggggaatttggttgctggcgttaccatcgtccagctcacgcagtaaggaggcgtcatgagccagatcagcaagacccattcgcgcctcgcaggccgcaatgcgaaacctttcgacctgaaaaacatcaccaatgacgccgtggcgtctctgagtcgcatcggcctggtattcgaccacgccgtcgtccaggaccagatcaaggccttggcgaaggccggcgcattccgctccggctcggccatggacagcaacttcaccgccccggtgaccacgccgtccatcccgactcccatccagttcctgcagacctggttgccgggcttcgtgaaggtcatgaccgctgcacggaagatcgacgaaatcatcggcatcgataccgttggctcctgggaagatcaagaaatcgtccagggcatcgtggagccggccggcactgcggtggaatacggcgaccacaccaacatcccgctgaccagctggaacgccaacttcgaacgtcgcaccatcgttcgtggcgagctgggtatgatggtgggaaccctggaagagggtcgcgcctcggccatccggctgaacagcgccgaaaccaagcgccaacaggcggccatcggtctggaaatcttccgcaacgccatcggcttctatggctggcagagcggcctgggcaaccgcacctatggtttcctgaacgatcccaacctgccggcgttccagaccccgccgagccagggctggtccactgccgactgggcaggcatcatcggcgatatccgcgaggccgttcgccagctgcgtattcagagccaagatcagatcgatccgaaggcggaaaaaatcaccctggccctggccaccagcaaggtggactacctgtcggtcaccacgccatacggcatttcggtttctgactggatcgaacagacctatccgaaaatgcggatcgtgtctgctccggaactgtcaggcgtccagatgaaagcccaagagccggaagatgctctggtgctcttcgtcgaagacgtgaacgcggccgtcgatggaagcaccgatggcggcagcgtgttcagccagctggtacagagcaagttcatcaccctgggtgtcgaaaagcgggcgaagtcgtatgtggaagacttctccaacggcaccgccggtgcgctgtgtaagcgtccgtgggccgtggtgcgctacctcggcatctaaccgatgcctattcaccaaaggccggaaacccggcctttgttcactctgactctgactcggttgtaggggccggttagggcataattactaggactacgccaatgactgtttacatcgtttccgccatgactcaatccgtgtcttacaatgcgtatgacacctctgatccgtccaatcctcgcctccagcgaaagattctgattcgtggccgcgccggcatcgcatccgaaacttccggcttcggcgacatgatttccgacgcggccgggcgtccgatctggaccccgcagggcgattgcactgccgtgagcgattcggatttcgagctgcttcaggccaataagattttcatgcgtcacatggacaagggttatctgcgagtcgtgaagacagacatcaccagtgaccaccagcggatttccaaagagactcgcaccatggagcgcgatggattccagcctctggacgctgatcgtctgcagcagaaaatcaaggtgaccaccgccagcgcttcccaggaacaagagttccggatttaaccgagggtttcggtatggtgattttcgacgaacataagtttcgcacgctgtttccggagtttgctgatccagccgcttatccggacgtgcgcctgcagatgtatttcgacattgcgtgcgaattcatttctgatcgcgattcgccataccgtattctcaatggtaaagccctggaagcctgtctgtatcttctgaccgcccacctcctgtcgctgtccacgatgcaagttcagggcgcggctggaggtggcgtcactgccggtgggacccaaggcggtttcatcaccagcgctacggtcggcgaggtcagcgtggccaagctcgcgcccccggccaagaatggttggcagtggtggctttccgggacgccttatggccaagagctgtgggcgctcctcagcgtcaaagcggtgggcggattctacatcggcggtcttccagagcgccgaggcttccggaaggttggagggacgttctggtgatccctggtgcgaatcttctgcgaatggccttcggggtcatcggcactcaaattgtgaaatatcgcaagtttgagcagcgagtgaagaatgatcaagctcagtacgtttccatgttcgaggagcctttcgacctggcagcgtctgtccagcgagtccgacgcgatcagtatgtccagtttaatctggagttccaacggaattatgttatgatcttcgccaactttgagatggttgacttggatcgcgatgtggccggtgaccagttcctctggaccggaagagtttttcagctggagtctcaaggctcctggttttatcaggacggctggggagtttgcctggccgtggatatcggtgcggccaagctaactgatgacgggaaaccgactttctaggtgatgtatgtttgacggcgaactgatagcgaaaatggttgtcgagctgaatgcggcgatgacatctgctcaagaggctttgcagttcccggattttgaagtcgtccagaaagctcagccgacccaacagggaacgtcaaccaggccgaccatctttttccagaaactgtttgacattcctcgcggctggcctgccaccgattggcacctggacaacacgacccgcaaatatgtagaaataactcgacagcatgtagagacgactttccagatcagttcccttcattggcagaatcctgaaataactcacgtggttacggcttctgatatcgccaactatgtgagggcttatttccaagctcgatccacgattgagcgcgtaaaggaactggacttcctcattcttcgcgtttctcaaatttccaacgaggcattcgagaacgacaatcaccagttcgaattccatccaagttttgacatggttgtaacttacaaccaatatattcgcctgtacgaaaacgcagcatattcggccgatggggtattaataggcgtatgagtctgaggcgcgattcagagctaatcgccgcgcacctccagatgttaagagccatgcgcggcaggtccgtttcagccggatggtattccacggctcgatatcctgacaaggcaggcggatcggtcggaatacaagtagcgagaatcgcacgtctcaatgagtacggcggaactatcgaccatccgggcgggaccaggtatattagggacgccattgttcggggccggtttgttggcgttcggttcgtcagaaacgactttccgggagaaaccgaggtaactaagcctcacagaattaccatcccggctagaccgtttatgcgatatgcttggaacttgttttccgcagatcgcgccgcaatccagaaccggatagccatgaggctggccagaggacagatcactccagatcaagctcttgcccagatcggcctggcgttggaaggatacatagccagaagcatcaggaccgggccatgggtggctaactcagcatctacggtcaggagaaagggattcaacagaccgctggtcgatacggctcatatgcttcaatcgattagcagcagagtaacataaccaggagatcatccagtgatcagtcagagccgttatatccggatcatttcgggcgtaggcgcaggcgctccggtcgcaggccgaaagctgattctgcgcgtcatgactaccaacaacgtcattccgcccggaatcgtcatcgagttcgacaactccaacgcagtcatgtcttacttcggcgcgcagtcggaagaataccagcgcgctgccgcatatttcaagttcatcagtaaaagtgtgaattcgccgtccagcatcagcttcgctcgctgggttaacaccgccatcgcgccgatggtagttggtgacaatctgccgaagaccattgccgatttcgccggcttctccgcaggcgttctaaccatcatggtcggcgcggccgaaaagaacatcaccgccatcgatacgtccgccgcgacttccatggacaatgtggcgtcgatcatccagaccgaaatccgcaagaacaccgacccgcagctggcccaggctaccgtgacctggaatcagaacaccaaccagttcaccttggtcggcgccaccatcggtaccggcgtcctggccgtggcgaaatctgccgacccccaggacatgtccaccgccctcggctggtccacctccaacgtcgtcaacgtcgccggccaggctgccgatcttcccgacgcggccgttgccaagagcaccaatgtcagcaacaacttcggttcgttcctgttcgccggtgcgccgctcgacaatgaccagatcaaggccgtgtcggcctggaacgctgctcagaacaaccagttcatctacacggtcgccacttccctggcgaacctcggcactcttttcgacttggtgaagggcaacgccgggaccggcctgcatgtgctgtcggcgactgccgccaacgacttcgtggagcagtgccccagcgagatttttgccgccaccaactacgatgagccgggcgcttcgcaaaactacatgtactaccaattccctggccgcaacatcaccgtttccgacgataccgttgcgaacaccgtcgacaagagccggggcaactacatcggcgtcacccaggccaatggccagcagctcgctttctaccagcgcggcattctgtgcggcggtccgaccgatgcggtggacatgaacgtctatgccaacgaaatctggctgaagtcggctatcgctcaagcgcttctggacctgttcctgaacgtcaatgcggttccggcgagcagtactggtgaggcgatggtcctggcggtgctgcagccggttctggacaaggcgaccgccaacggcacgttcacctacggcaaggaaatcagcgccgtccagcagcagtacattacccaagtcaccggtgatcgccgtgcctggcgccaagtccaaaccctgggctactggatcaatatcaccttctccagctataccaacaacaacaccggcttgatcgagtggaaggccaactacacgctgatctattcgaagggcgatgccatccgcttcgtcgaagggtcggatgtgatgatctaatggtttgcggcggactcgatccgccgcaaccttccataaatggagtgaggagtaagcaatgatcaacatttctgcgttcggctcgatctgccagttcacggcaagcagaactttcccgaacggattcaccgtcaccgagttcgccgacgatgccgatcccatcgacagcccgccgttcaccgctgccgataccggcgtcggtctcaacggtgacatggtagtttggaaccgggcgaacatcctggaagtagtcgtcaacgtcattccgaataccgagggcgagcgcaacctggcagtcctgctggatgccaaccgcaccggaaaggacaagtcgggcgctcgtgatgtcgtcggcctggtcgtggcgatgccggacggcagcaaaatcacctgcaccaacggcaccccaatcgacggcgttctgatcaacgcggtggcaagcgtcggccgtctgaagaccaagccgtatcggttccgatttgaaaaagtgatcaaagccggtactagctgatgaagaaaattccgctgacagcagtccccaatcaagcgatctcatttaacgccggtagcagctattggaagattcgcctgtaccagaacatggacatgatgaatgccgatatcagccgcgacggcgtgatcgtttgccatggggtccgctgcttcggcgggattcctcttctccagtacagccgtcagtaccgacctgactatggcaatttcgtcttcgaccgcgacgccgattggacattgttcggcgacggcatcaacctgttctatctggacggcgccgaattcgccgagtatcaggcgcttgccacgaggaaagaatgagcacatcaacgatcagaaccgggacgaacaacgatatccttttggacgacaatggaaacatggttatcctcagggatgtcgaagcgtgcgcccaggacgttcgggcggcgatgctcatgcgcaccggcgaaaacattttcgatgtgaactccggtgtgggatatttcgaatacatcttctcgccgcagaaaagctatgatgacgctcgcaaatccatcgcggatgcaattttgtcctcgccggatgtgaccggcatcgagcagctggacatcgacatcaccggggaagtcttcggcgtcgatgctaaagtcatcaccatccacgggcctgttaccacaggagtttgaaatgagcacaatccgcatccaatacgccaacggcacccagctgttcctggacggcaaaaatccgccgctcctggaccctctaccctcgttcaacccgtctgtagaagatctggaaggcctggaccgcgaaaagaacaccggcaagggcgactcctcgccggccggtcttcccgttcccccggtaaacgtcgattccaatgtcgacaacggcggaaccatcccggctccggtatcgaccgacgctcctgcggccgaatcggccccggaaggcgcccaggaagctcctgcagcgggacaaggcgacgagaaaggcgccgaggaagccccgactaccgccccagtagaaaaggccgaggaaacggcctcgccggccgctgaagaggaagccccggctcccgccaaggccacctctcgcaaaaccaccagcaagtaaggactcgacatgatcaacgtcagcggcttcggcacaggaattgtaatagtttccgcctcatcgttcccgatggggttttccttgtcgaagttcgctgatgatgagagtccgatatcctccaaagagctggagccgttcggatatgagatgctttatgacggcggcctattcgcctttgacaaggctgctcctctggaagtatctgtatccgtcatcgcagggagcgaggatgatattaaccttcgcatccttctcaattccaaaaagggatcattccgatttcttccgggaatcatcccggatatgacgactctcgtggccactcttcctgatggcggccgcaccgttctgtccaatgggactatcatcaagggtccggccatcgacaccatacagaacaccggacgacgcaaaggcaacacgtacacttttgttttcggtagctatcttggcgcccagactgcgcgtcaagccatttctaacgttatccaatcggtactggaggtggtctgatgttagggattttcaccagcctcctaagttcgcgatctttttcgattgtggaccaaaacacaaaccagctagttgctgcggatttgaggataagccgggtcaacacccggttttcttctgtagggcaacgccacatgctggaagatggcaagaccaagatggattccagaacgatccaccctatggaaatcatcgtcgaagtattttgcccttcaattgatgtcgtcgatcagatcaatcaattgctcctggatcgcgacactctgtacaaagtcatcactcgcggcatggtattcgaacggatgatgtgtaccagcgaagcgctcaatcagactccggatatgatatcggcgactcctgcgcggctgacattctcccaagttctcgtccagaatcccaagcctataatgttcagaaatgcaggggactcttctatgatcgaccgagggctggccctagctgaagacgtggttggctcggccggcgatctgttcgactacgcagtaaacggcgtccagaacgccgcagacttgttctgaggtgccaattgaactctttcctcaagtctattctcaacacgcctactctcaccatacgtgatgatgttaccaaacttcctgtctggaagagtcttcaagtcaagaaagtggaaatttactcgccggcttccgtagtgtcgaaacctctggcgacgaaagaccagacggaagctcaagtgtacaccgaagctctggacattgatgtgaagaatggaaagatcatccaaccggtgcgactccgcatcaatgctatctgtccggacctgtccaccgttgaaagtatcatgaacgctttcaatgataacacctcgactttcgctatcacttctaagtcgatattggccgataaaatggccatcatgacgctcgatgtagatcagtcgccagacatgttgaacgcggctgagatcaatatggaattcgagcaggttgagcctccagtattgaatgaattcgatcctgcgttccctcaagatagtccgacttatggggtacagattcaatctctttctgatgctaatttgctggatttgggcgccatcggcgattcgatatcttcggccgcaaaatcgctatataatcgcgtgaccagctacttctgaggatgtatcatgcttgaaatcaatcttcccgatggccgtcaaactcgcgtacaaatcgaggcgtggtcggcattggatggctgggaactccagcgccgtttcgtcgaattcgctgtcagccaagattccgacttccgccgctctttcaccatggaaatcctgggctatgcgaaagtgctgcttggcgacgatgacaccggcattccgctgaccaccgcggcagtcatcaacaaccacctcggccactggaagaacgtggaactggttttcaactctgttctcaagcacaatggcatcgacccggccacgcacgccgaccggccggactattgggaacaagtcggatcgcagatggccatcgcatttctggccgaggcgtccaagctcattggtccagcaatgaagatcgccgaaggactcgccaacaagccggagtgattcatgtctagtgatttggatgaattcatacttcggtatgaggccgatactgccagagccgagcgcaatctagagcacctccagaaccagatcaggcgcgtaaacagcgcatcgacgagtggccttcaggatttgcgccacttcgcagacggcgcggccaccgaactcggccgagtcgttcctcagattgattctgtgacgagcgcaattcgcggaatgaacgcgcaactggcaataggcgcaactggcgtggccctggtcgcggccggcgtcaaggcgttcatgaacaccagggaccagtacaatcagcagcgcatccaggcgatggacatcggcatcgccccggcacgcttggaagagtaccagagaaaattcgttcgccagtccggtggaaccatcagccgcgagcagggcgcggagatgaccaaaaatctggcagacactttccggcgtgcttatcgcgatatcgggcgagtcggcccggaagcgcgaattctgcgtatggcaggcgtcgatgtcgggagcttccaaaagggcatgcggccgctcaacgacatcattactgatctggccacgaaaatggccaagttgaagccggacgaaatttctgcctacgctgatgcccttggcgtctcgcgagactacctgagcaccctggcgaagatcggcccggccatgggaaaagtcactgagatgacgactgcggaactccagtccagggtccagggcgagtccaacatccagaaattcaacgatgccctggcgaatctcaaccagacgttcactaccctggaaaaccgagtcggcgaaaagctcgcgcctgcgttcaccaagctgatcgaaatcatcgacaagatagtccaggctattcccaatgaagtggaaagcttcgcgaaggacaccaaatcccgttgggaagatggagtgctcgggaaggcgaccgttggcagtgatatcctatccctcctcagccccggcgccctgctcggtcgcctggcagcgtgggggactcgtcgcggcatggaagaatcagggctcatcgacaaggacaaggttcccggcgctcaaaccagcgaagacctggccaagaaacaagaagaccaggacaaggctactaagtccatgaaagagctagagaagctggctgaccagaccacgaagtctaccaatgattttgcggtggcgatcaacatgttcagtggcgcggtatcgtcgttcgcgaatgccgttgacgagcgccaagcttgggcagcctgggcgggggaaattggtcgggcggtcggcatgggaagcaccgcgccgacttcgcgggccaccggcgtctatccgcacgcgatctatgatcagtcgaagagtggcgcggccggacaagtcttcggcgagccaatcggcgcccagtctcttcgcaatcgcatgttctcgccgcagcgcaaggccgagccggtcaccgttccatcgtacatcaacgacatcatcaaagatgcgtcgaagatgtacaacattcctgagctggacatcaagaaactcatatacaccgaaagccgattcaacgccagggctaccagcgaagccggagcgaaaggcctcatgcagctgatgccggaaatcgccaaggcgtatggtataactgacgtatatgaccctcgccagaacatcctcggcggaacgcgcctattgcgggaaaacctggatcgagccaatggcgacatgcggttggccttgacctactatcacggcggcctcgatccgaagaactggggaccaaggactcgcgcatatccgggtttggtgatgagcgctccaatcgagctgatggaagaggctcagcgcaagcagaaggctgcggccatgaccgtcgccaacgagacgttcgccccggaaggcggcgacatggacattcgtccctatgatggcgggcgactggaaaccccagaccagggcaagaaggaagatgagcgccgcgaagcccgtcgatatgacgacagggttgtgcgaccggaaattcgcatcatcgaccgcatgccagaccgcagcgacggcgaaatcctcaagatgtctaggcgtcaagaggccgaccgggcagattctggattccggaagttcccgaatcaagttcgcggcgagaccaagcagaacatccaggctcaactcactgcaggagccatcgcgcaagtgatcggcgttaaccccaaccagatcatgcgccgcgaaatcagccgttctgacttgctgttcggatacaaccaagccatcctgggcaagcaacaggagatcaaggccgccgcgaccgaagccaacaacgtattcctgtctccagccaagcttgccgaagccacggccaaggtgaacgccgcatcgcgagaaatggatattctcaggacgtacggcgagcaacttctgaagagcgctccagagcgcggccaggagctgaccatcggtcgaatcgatatgttggtaaacgttaccggcgcgaattctccggaagaggctcgcgagatattcagtaagcagactgcagaccagctgactacggcaatccaggacgctcaaaacgattccgcaactaagatactctactgatgaaaaagagaattctgcgagtaacattcaacatgccttatggacccgaagtcatccgcgaagatctggatgttcgggtccggattatgaaggctgcgttgcggattcagaaccgggcgacgatggaaattttcggcctcaccactcagctgcgcgagtctcttctgtcgcagttcaccgcgtggaagcaccggcaacgtcaagtgggcagggaagatgaattgatgatcaaggtgtcggtggaagccggctactctgaccaggggcgcgagcaagtttccagagtctttgtcggcgaagtcgccattgtcgatatcatttctccgccaccggacattggaatccgcatccagtgctataccaggcaaatcgacaggacgaagaccattcggaatatgccgccagccaacacgacgtttgtcaagttcgtcgagtggggcgcgaacgaaatgggattgaacttcatttgcgataccagttacaacgatcaagttctgaagaatccgggccggtctatcactgtcgcgtcggcaatcctggcgtcgattcaggatatgtacatgccggacgtggccgcgttcgtcgatgatgacattttgatcgtgaaggaccgcgacaaagtcattcgaccggatgaggtgaccaacgtcaactcgttcgttggaattccatcttggtctgaatggggcgttgaattccagtgtctgtttgagccatcgattcgcgtggctggaggcgtggcggtcgaatcgctcatgaacccaagcgtcaatggcaactatgtaatcactgctctggaatacgatttggccagtcgggatcggccgttctatatcaaagtcatggggagcccagcagcataatggccagggaaatcaaaccgttcaacatgttcggcgttcactatacttcgcggcaattctctgctgtcgatgggctcagcatgatgtcggaaattcagagtgtgccgccagaagaactgctcaagggaaccagtgtattggcgcatccggaagaccatccggaaggcatctggcttccattgactgctgcgaacatcaatctttatgtcgttgatcgagcgaaagtaatagctcccgtacaagttcttgcacttctgtccgaagtggtaatcgactggaactttggttttctcaaagactggaccggagtcaagattccatccagatttgtcgaagatatcaaaagcgtgaagacggcacattcaccttctgtagttgcgagcttggtcgcgaacggctcagcctctatgcgcgagttggaagagtattattcgactcaagatgcgtttaagatgatagacatcatgacggcgaagagcgtgaacgaggccttggcgtccgaagcatcgcacaacagaatcaaaaagggataattcctaaccgggcctgggaaggctatactagacctgccaaatcagaggctttcccatgtccaatatttctctaacatccgcaaaagctcccgacaggacgcgactgatcgccgctcttgacgctcggtcgcggcgggatgctctagatttcgaagtgatgataccggctcaggttgttcaatatgaccgggccgagaacatcgcgacgattcaacctctcatcacctgggttgacacggaacacaacgccgtccaaaggcatcagctggttgatattcctgtaatttccatgggggctggcggcttccacataagtttcccgattcagcaaggtgacataggctggatttatgcggccgaccgcgacacgtctcagttcctggagtcgttgtcaatgtcgaagccgaacaccggccgcatccataagttcgagcatggcctgttcatcccggacgtattccgtcgatacaccatcaattccgaagattcggccgctatggtcatacaatcgacttccggagcgaccagaatttcgattcgcggcgataacatcaagataactgcgccgtcgaacgttactgtggatactccgcaggcgaacttcaccggcaatgtgactatcgctaacaccctggtcgtaaacggcatcaacgtgaacaaccacggccacctcgaaaacaacccgcctgatgcccggacgaaaggcggcatgattgcttaaggagattttcatggcaagttttgatttttctgatttaacagcgggggggggttgtaatggctaactacaactacatcgtcgatactggtgtgatagtcgcagataccgccgacgttctgagcgacgttgaggccgagttccgcgccgccctcggtgccaatatcaacttggccgcgagcactccgcagggatcgcttgtcgcggccgaggccattgcccgttccagcgtcatgcggaatgaagcgcgaattgccaataccataaacccgaacgtttcattcggaacgttcctggacgcgatctgtgccttgatggggatcgagcgtggttctgacctgtcaaccttcggctatggagttcaagtcaccggccgcagccaaacccgcatttccacagggtctagggtccagactccggccggcgcgatcttcacagtgctgagcgatgtcacgatccctgctggtggtgtcgcgaccatcgatatcaaatcgcaggagtatggcaacattcctctgccggtcgggaatctcatcatcatcgacggcacaattggctggtccggagcgaaagtcatcgcctccactcgcgtcgatccgggcagccgccaaatgagcgatgcagaactgaagaatgcccgcgttaatcgattggccatccaaggccgcaactcgaccatggccatcaaggcgtatgtgagcgccgttccgaacgtcacgtcggtgaacgtaatcgagaacaacaccggcgccgttcaagtggtgaatggagtatcgtttacccttccgtatgctgtttgggtctgcgtcgccggaaacccggataaacaagcagtcgccgatgcgttgtgggccgcccataacggcggaactccatgggactatggtgcgacgaacaacggcgtcccggtggacggtccgaatggcgttcctgtgcgcgatccggcgtccggtcgaaagtatgtcgtgaagtggaccactccaattatgtacgatggatatgttaacgtaacagtccagcaaggttcctcctctgtagctccggaagccattcagaacgcagtggtcaattacgcccaggggaaagtggaaggggaagaggggctggtggtgggcgcgagcctgtcggcattcgaagtcgccggcgccatcgctcgcgaaattcccggcatctacatcaaactctgccaggtcgcgtgcgtcgcggctggctcgccggctccggcgccgggcgatttcacttcggaatacgtcatgagcgcattcggccaggctaccatttccgttggtaacgttcgggtgactttcgtatgactctgcctgcgtacaattcggacatccaacaagctctgaaatggctccataaccaggcgcctggaatcaccggcctgatccagagaaaggcgcaatggtatgacagattcagccgccaattctgggccaactgggagcgcgacgttttccacttgaaaactgccaacccgttcggccttatggtttggtgcatcatcctcggcacgccttcgaaagggttcggcctttatccaaaaaacagttcttgggcattcggtcggctacgccagaacttcatctatagcggtacacaagttccgccaccggcagacgcatcgcctggcggcaacttctacggtggcggcaatgccgaaattctcaacttggacgaaatcaggaaagtgcttcagctaagatatgtagcgctgatttctaacggctcgattgcatatatcaatcgcatgcttcgttacatattcaatgatgatgagccatgggacgaggcgaccggtctgtacttctatctcatggactcaaccggcgagaatggccctgtggagaacttggcgatatatcggaaagattgggaaggtatggtgctgttgtccagttcgcccagaacgaaccacgtgctgacatcgacccctgccagcgacgccgattggccgggagtcgatccggccgcgagcggtattccggtaacggtcgaaacggcgtccgctacggccccggacggctccgctacggtgtgcaagcttactaagccggccgggagtaccgcttacgtctccgcgccgatagatgggccgctggggtccggtagcactgtaacgttctcgttcttcgcgaaagccggctccacccgtttcattgcaattcagtcggctgccgatttccccagtcgagccgatgccgttttcgacctggattccgggaacgtaatcagcgatcagatgctggacagcagcgtggtaagcgcccgaatgattcgtctggaaaatggctggtggcgttgcgttctcacgaccaagaccgtcagctcttcgttccgcgctgcttacgtcgctccggcagaaaccaacttcagctggattgattcgaattccagcgcggcgattgacgtgcttatctggggcgctcagatcgaactgggtgatactccaactggatacttggaaactaccggggcgcccgtaaccatgaccgattatgttttgcagaacgcccagaccggaacagtcaagttcacacagccccttccgaccggagtagaagcgtattggactggagactggaaaggcgggactgcagcagaaccggccagattcgcagtcgggaatgggactcaggatacgtttactctgtcagaccccgcatacatcggcctacccaccagtggggcgttcaagctagaatacagggttggtccggcgcttaatctgtcgccgcaattgatcaacctcatgaatgaccgggcagttggtattatgccgacttgcgccggttgcgatgtcaaagtcattcaggagtaatgacgtgatcacacccgaactgatccccagtccgtttgccgctcagggcgataaagacccgattccacaaacctcctccactggcttcgcaaaccttcgcgacggctatacgccggactacgaaatcagcctggcgtcgaacaacccgcaggccaaagcggtcgagcggaaaattcaaaaccaactcttcttcatcgcgacccagaacgcacaggcgtggcagcgacaaatggcgccgccgtggtttcagggcatgcctggcggctacgaacagaatgcagaagtcgtgcgcgtcggaaatgacggcataatgcggcgttatcgttccatggtgaatgccaatgcgagcgaccctctcagcagcacgacttgggaagaacaacccgcatggtcggcgatgcgctccaacatcccgatgccggccggaggcccaggcctatcttctggcggagaagtcatcacgaccggccgcaacttcaacgacctgttaaatgggacgtgggagttcttctctgattcagtggttatcgcttctcagaatgcccccgtatatccggcgtccgctggtgccgctgctggcatgttggaggcgaaatcttggatatccgggtccaatacgttctgcgttcaacgctacactgaccgcgtcgggaacgtcgctgtgcgcgggcttaatgccggggcatggaccaactggatgtatgctgtaaacgttatggccctccaacaaggtcgagtgacctatggagtcgcggccggcccggcgaacgcttacacgttgacgctcgttccgcagctccaaggcggcctggtggatggcatgattcttcgggtcaagttcaacaccatgaacaccggcgcctccaccatcaacgtctccggactcggcgccaaagccatcgtcggcgcggccaacttccctctcaccggcggcgaacttggccaaggactcatcgctgagcttgtattcgacgcagcaggcgaccgctggagaatcctcgcaggcgcgccacgcatccaagttggcaacgccgatcaagattaccaggcccccagctggaaacaggtgaaggattacgtcgcgtcccaaaagctgactgaagtggactgggctgacgtcgtcaacaagccgaacgtcgctatccaagacactacgccgtggttcgccaatctggagttgtctgacgctcggcctttcatcgatttccacttcaacaacaaccgcgccaaagacttcgactatcgctttatctctgaagctgatgggtcgatggcgttctactctcgccaggggtcagctggtcctacccaggatatcctgttcagcaggtcgaatgttacattcctccagccgcgactggatgttgcgaaaaacctcgcgtacatcgcgaactctggtcccctttggcagaacaccactgccgatcagcccggttggaagttcacattcgcgcaaggcgtggacgcgaacaacaacgcggtgatcgcagtcaataccaccaatcctgacggctcctatcgctcacagataatgcgttgggactgggcgtccacgaatgtaatcttcaacaatcgtccgcttttcgccgggcaatatgttccttgggattctggtaacttcgacccttcaacaaagttgacagtcaatgccacgaaccagatcgccgggcccaccgggattcggaacaccaacggcaacaccggcaatatgaacacatggggttccggttccacgacggcatcctatggcaatgctgccatccagatcttcgggaaagggggcggtgagcctgccgcgatctattttgacaactcccagaccggatggtatctgggtatggacaaggatggacagctcaagcgggccggctggtcactcggcaacaactcttatgtcatcaccgatgaattgaacatcagaaaccacatcaacggtatggcagcaaggccggtttggggcggaaatgaattctgggggccgtggaacttcaatccgaacactaagcttacgttgggcgcattcaacgatagccaacacaccaggatggtaaacagcgccgcgaaggacgtagggatagccgccatgacaagttatggcgacgcggctatgtcgttcttcaactatgaggcttcgacgccgaccgggaatcgtgctgctgtaatttcgtttgttcgtaacggggcacgaggcgttctgttcggcctggacacggacaacaagctgaaatggggcggctattctctaggtgccgtcgcgttcgagattgccgactccaacaacctcatgagcctgtggtcatcccacgctgccgcgccgaactggaacgggcagaccatctggaggtcgggaaacttcaacccagacaccaaggcgactttggcagctcgcaatacgacgtcatcccctacaatattcagttatggggcgtccggaatcgcatcaaccggacaggtcggtgcgttggttgtggaaaacaacagcgtcaccaataccgcagccgccatcacgttccattcgccgcagaaatatcaggtcaacttcggcctggatgcggacaacgtggtaaagatcggtggcggcacaatgggcggcgtagcatatcccatcatccactccggcaactacaacaactacatcaaccaggcgctggttcaggtaggtcttggcggcgtcggttcctacgcgatccttgccgtatttgacacctccgcgccggcagcgtccattgcccccggaacgatcatggacagttccaagctgttctattcgtcctgcgattcgacctatcgcagcagcgccagtccgacgggcacctggcgctgcatggggtatgtgtataaccgagactccaccaacggcgactcggcatccctattccagcgggtaacgtaacatgaaatggacgcgaatcagaaaccctcgttggctggatgagacaaatattcacgccatggttactttcgaggggattggagaagttcctttcaccgccaatccgcacgacgtggaggcccacggaagggccattcacgctgcgatcctgtccggggcgcacggacctatcgccccagtagactcgacgcgggagcaggccttgcaggacgctatacgagcaagggaaaagcgggctatccttcgcgatacccgatggcccatagatcgtcacgacgagcagaggcggctgggtatcgaaaccacggacgggcctgggctgatagcagcccttgttcactggaggcagcagattcgcgattggaatagcggggatcggccgcgacttcccatggctctgaaagcaatattcaaaaatcaggagtattgatgaaaatcacgaaggatattctgatcatcggaaccgggtgcaccacggatcgggcgatcaagtggctggatgacgtccaggcggccatggacaagttccacatcgagtcgccgcgagccattgcggcctacctcgccaacatcggcgtcgagtccggtggcctggtaagtctggtggagaatctcaactacagcgctcaaggattggccaacacctggcctcgccggtacgcagtagacccgcgagtccgcccgtatgtcccgaacgctctggcgaaccgcctggcccgtaacccggtcgccatcgccaacaacgtgtacgcggatcgcatgggtaatggatgcgagcaggatggcgatggttggaagtatcgcgggcgcggcttgatccagctgaccggaaagtcgaactattccctgttcgccgaagactccggcctggacgctctggagcaaccggagctgctggagactccggccggcgcggcgatgtcttctgcctggttcttctggcgcaatcgctgtatccccatggcggagtccaacaacttctccctggtcgtgaaaaccatcaacggtgccgcgccgaacgatgcgaaccacggccagctccggatcaaccgctatatgaagaccatctccgcgatcaatcaaggctcctgatattcaaccccaaagaaaaggccgcttattcagcggcctttttgctttccggctttgcctcttcaaccatcttgacctcaaccggcgcggcggactcctcctgagtgatcgagtccacatagttccctagtgaactcagaacgccgattaacagcgctctcaccaccttgtccttgactgtctcgcctatgatcttggtcagaacggatattaactcttcccggagtctggggctgattcttggccgaaagcgcttgcgatgctctttgcgtttcatgtttagtcctctgtctgcggtcttctcctcaccccgataattgcttggggatgctttgtgttaatcggaagggtcgggcgctattataacccgtcgaaaatgctagcgcttaactgtttaacgatacgcaccgcgatattaaatcgccttctttctggccgaggaactctggcggccgggtccggtctaaggcctaatttgtcgacatcaaaacgagaaaacccggatcgcctgtagggtaaggcgtccgggtttatttcgatctagtgtccgctagaatcagtggcttccgccccatccgtccagccagcaatcgaagacggcgtgtctcggcttgtccttggcgccatgggagaagtgcttgaatcggatgacctggcccttgaggtattccctgtcattccagcggcgctgtttctcgtcgtgggtcaggctggaggccgacacgttgaaggtgacgccaggccacagacgttcgttgcggcagacgaacgcaccgaccattccggacggcgacaggttttctgcgtggctggagcgggccgtgtggccaagttcattgatgaaagcttcattgttgttgtgcatcaactcttccacgtcgatgatctcagcttcatcgtagtcgtatcgcttgactttcacacagtgcccttccttggcagtcgagcgaccgaacttgtacagtccatcagcgcgcttgcccatggaaccttcgaatcccagcattgtgtggcggcgctcgacttcgctgaactgttcgatggaagtcaccagttcctgctcgaccaggtgaatcctctcatagccgaggcagtgacgaaggaagttcacgcgctcggccgccctggctaggcgatcttcggtcggtgcgcgaggatcggtgaaatcatcgaacacatggaaagaccagtccggctcaccgctgtggcggcgaagatcgccggacgacttctggaaaactttcgggtcgctgatgtcgccgcagaccagctcgccgtccaggccgttgaacagcttatcgctgaggtattcgtagatcgattgattgttctgccgcttgagttgacgggtcagcgcctcgccttcgaatacgaagcagcgaaatccatcgatcttcggtgagaaatacatcggcagttggccttccagcagcttcgggtcaaaattcgatgcgagcatgggtttcatacagttctccagaaaagaagcccggcgaaccgggctaaatggcggtaagccggctcagatggtttcgttggcgtggttcagttcggccatgatcgacgcatagcgctcgtccgactccttgatgaacacgccgtcgtacatgacgcctttgcgatccttgatggtgtcgtaggccgcagcgtagcaggcgagcatggtagtgtcgtgctcttccgtcacatcgaacagagtcatgacggccaagaccaggctcttgatggctaatccatgatttccgcgtgccagcgcgccggccagatgtccgaggtgctgcaaatcgtcgccgtaggtgggatgaacttcgacagtctcgctgaagacagatagatgatcgaacagattttcaccgagttgcgcggccatgatcgtggcaaccaccattacgtcgccgatgccgtctttcacctcgactggtttgttataaacccaggcttcgcaaacttctgcgaactcttcgaccaacttcaggaattgatccttggccgaagagcctttgatcaggttccggtcggaaccccatttaacaaccaggtcgtgaagttcgcgactcatgatttgttcgatattcattctttcgattccttttggatttgggattttactgcgttgattatggacgccgtgctctggcgcgatccatccttggtggtgccgaagtagaaggccattaccgacttcagctcggcgaaccaatagccgatgatggtgccgatggcgacagaggatgtcgggtccatcagagcctcgcggccgaatgtgaagattgcgatgatgatgagaatggagcctgtcagaagagcgaatgtaatcgccgggcgaacgaaatcgttctgctgcgccgcaagcctcctcgcagaatccctgtcggccgcctctgcggcgaactggctaagctcggcctggagctggttctgctcagactgaaggcgattttgttcggcttgaatcgcaagctcctggagacgaacgcgctcggcgctctggagttcggcgagacgcgctagagcttccggattcgcgtctagagcgctagcgaccgaggctgggtcggccttcgaccccagcgccgtcgcgacgatagcgcccacggcggcgcctgcaggcccacccaggagcgaacccagagccggggcagccgcgccgatcttactacctatgtctttccagtccatttcgactcctcaaaagaaaggcgccattacagcgcctttctccggccggtgatttagaactcttcggcttcggtcgcatcgccgacgccaccggcgtcactgcgaggctgttcctgcttgctgtagtccaccttcacctcgccgccgacgaacgacttgtacaggtcggccgcagccttgaagtgatccgggttcttcaccaggccttccagctcgaactggacgccggaccagctacccttgtcgttcgacatgcctacggtggtcatgcggaccaggttggcgaaagtcggcggggtgcgcaggccctgcggggtctgaacctttttctgggacagcgcggtcatgagcttcttcgaggccttgatctgcgaagacgacagggagatcagagcctggccgaaatcgccggtgtccggatcgatgacgatgacgtaatgaccacgggtgtcggcgaagtagtcagacttcttgtcgctgaccgaaccgtcttcgttcggcgcgtacaggcgtccttcgacttccttcaccttggtcgggtctttcatcatttccttgaagtcttcgacgctgatggcgcccttgaaaccgccctcggcctcgcgaccggcccagcggatgaactcgcggcggtacgcggccggaatgatcagcaggccggtcttgccgtcgtaaatcttgccggtgacggtgttcaggaacatgccggccttcgcgccttcgatgtacttcgggtcatcttcatcgacctggggagacatcttctgcagaacctggatgaaggggatggcataggactcggcatcggcgccttcgaagcctgcgccgtcatacgatcccaaatccatgaagtcgggaacttcggtagttgcgacggcgccgccaccggtggcaactgctacggccttggtttcttcggttgcttcggaagtttcggttttcttaccagccatgttaggctccttgtttgtcgaatttcagttatcgctaactgtgggtttataataacggaagttgcaactaagtaaagcaaattacatattaagatttgctctttttcaccttcggtttcgtgattttggcctctttgtattcgtgcacgccgatgaaatctggcagctcttcgcccttctccaaatactcgcgaccgaacgcctggagggtctggtagtggacatcgcggttgatggtggcgtcatagccggcctcaatgatagcctcggccgccttcttcgcatcttccatttcgccgcgaccgaattcggccagaaccttggtcttgatgatgccgtcgttgtcggtttcttccagccacttccagaacttcgatttgttctcttctttgacggaaatgatggccttcggctcgactttgaccgtgcgaccgtcagccagagtagtggtcttttgaccaagctcctccagcagctccggaatggtgttgcgcttgagggttttcagctcctcttccttttcggccagcgccttttgaagatcgaggatttcgacgtccagctgcgaagccttgtccaccaggttcagcagtcgatggccgatgtcggtggcttcaaccgccatttcatccatgacgccaaaatagtcgatttcgcccggcgcgttctctttcaagtattccgggatttcaagctcttgctctttcatgtccgcctccaacttagtgatgttcccttacttgaaccaagtattgagtagatattatgccgcatcttccttgatacggctactgatttacatattaaatttcgtcgcgagtgctaacgtcagcctcaaacacgccatcaacgacataacttgccaggttgcgtttccactccaagctgacctggattttctcgtcgatggaatccagacagatcaggtcgaagtacagaaccgagttgacggtgccgattcgatgatttcggtcttcggactgcatccgcagctcgttgtcttcgtcggtcgtgtagtagatggccacgtctgcggccgtgagcgtgatcccgatccctgctgcggccgggttgcccaggaacacctggacgcgcttggcctggaaatcgtcgatcaatttttcccgctcggcctctttggtctcgccgtagtaggtgccaaacgaaatcccttgggcctccaaatacgccttgatctggtcgatttcctgaatgcgcatggcccagacgatgatggaacgctccgggtcttcctccagcagaccctccagaagatcagtaaacacggcgaatcgcgggttgtcttcgggcggcaggatcaccggctcaccatagacgttgatatagccggacgcaacttgcttgagcttcgaacgcgctgccgcagcatcgaaagatacatccagcatgaaatcttcgttctggagaacgaagtgatagtcttcttccacgcgctgataaacctttcgttgctccggcgacatttcgaagtatatgcgcttgtacaccttgtccggcaggaatggcaatgcttccttcttcgtgacccggaagctgtgcggctcgatcagggatcgcagcttgtccaggttccggaataccgggcgcccataatcgtctttttcgacgagttgcggcggcatggtttgcttcccttccagcttgcgcatgatagcgatcattcgaggatcgtcactgggcaccaagacggaaaattcggccacaaacgcccgataggatttagtccccagaattccattccgcaagaattggaactgcatgaacaaatcggtcggtgcccgcgtcagaggagttccggaaaggatgcgacgggccacggccttctcgcccagctttacgatctttttcgcacgcttggcttgtgggtttttgatcctcgttgactcgtccacaattgcgcaaactttgaatgtatcaaggaatcgctcgacctcatcatagccagcctggtggttgattgcgtcaacgttgatggcgaagacgcgaagaactttttcatctgcgaacgtctcggaatacagacggtccaagcgcgccctggcctttttggaagtcggtcggccgcgccaatccacgctcaaagtcttgatcgcgacgtgggtgggaatctcgcgcagaatccagttggtgtggacgcctttgggggcgacgatgagcagcgcgtccacgcgcccctgtaggaagaggcgaacggcatcggccaaagtcgtccaagtcttcccggtgccctgctccatcaggtatgcgaagttccgtttgttcagggacgctgccagggcattgaattggtgctgcatggcctcggtcttcatgcccttgacgggatatgttttggctttcatttgttctccaaatcggcgaggaactgaacaatgttatccagtccttctgcatagctcgcaacttccaccaagtcgcggctgttaagctcgaacaaatcgaacataggattcagcagcagccaatcggttccgatcttggccaggacgaagccccggccaccccagccgatccgctcccgaagaaaagggatttgcccaggttcgaaacatctggccattgggcaggtggaagcgcgtttcggccactcctccaaagccttgaactcgacccagaactggacacctcgacgattcaggcagatcgaatcggacatgccagaccgccgcgtctccagaaagtcgatcaggattcggccgagcgagcgctgcttaaacgcattcgccgctttcgtttcgcgatcattcatcaccttcgccctctttggaaactttctctgcttgcgatgccaactttgacttctcgcgctcggtcaatatccgcttgacggccttcacgatgaacatgtcgatgccgctgagcctccatcctttgatcaggaaccaagcgccggtcggcgtcccttcagcaatctgctttccgtattgcagatatttttcaggccgaattctgaaacgtatcggctgatcaaccgagtcatcaacgcacatgatgtcgagaaactgcgactggcccttgtacaccgggtttttcccttgatcggccctcttcttctggcgaatcggttcgttctcatcggacagaactttcttcaccatcttgacgatgactaggccatcgtctccatcgcggatatcccgaatgttctgaatggggtttccggaagtcactccaaccagctccggattgtcataagcatgaccccagagcgtgtgagcttcgttcaagtctgcgaattgcacttcagaattcgacaaactagctgcgactttctcccaatcctgaagcgtcttcaggtgggtgcctgccagctctttatattgagccttcagctccttaagatcggccttcaactccttcagatcggccttcaacagtttctccagttctttgtcttggctgattttcgccgaaagaatctgggcttccagagctgctacatcagcagccttgtcctcaacatcctgtgccgatatcgggcaattggccagggcgatcctcgcggccttgacttcctcgcgaagacgcaggaaccgctcggccttcgccgggccgaagcctttggcgttcatgatgccgccgatcaggcgcccgtccgccaccacccagttgagttcggaatgctccgggtccagggccgtatattctacgccttctttggccaattcgcgaaggatggacacagtttgctggtcgtctttcgccgcccgaagacacgcggccgcgtattccaggcgatgatatcgcttcatgtagcaggtccagtacgtcaccacagcgtagctgaccgagtgggagcggttgaatccccaggcgccgaacgttaccatttcctgccaaacacggtgagcatcgtccggggcgacgcctatggtcttggcgccctcgatgaacaattcccggcgcttgttgaaaaattcttcgccctttcgcgcagacatggctttccgaatcgccgacgtttgttcccagtcgaactgcccaatgtccttcacaatggacatgatctgttcttggtacaggaaaactccatatgttccagacagatactgctcgacctgcgggatggtataggtcacaggctcgcgaccggccacgcgctcgatgtatttggtggccatgcccgaagacaacgggcctggacgggcgagcgccgttatgtggtcgatgttttcgaacgcggtgatattgatcgcattggcgaccgagcggacggcctggccttcgaactggaagatgccggacatcttgtcttcgttgagaatgtccagaaccgccttgtcgttcaacggcaagtcgtacagctcttgcgccgtcacgcaattagcatcttggatgacgcccagagttcgaagcccgagcgcatcgatcttgagaagattcagatattctgaatcgggcttgtcgagctgcgcgacaccttcagaagttaccgtgcagaagtcgatcacttcatcgttgcagaccaggatgcctgccgcgtggacgccggagtgggatgggtggatttcgaggtcgcccatgcaggcggacgcaatctcatacttttcacggaagtcgcggccgggctgagttttttcgaaagtatcctccaatccttttccatatcgttcgtccgccgatgtatattcgatgatcgagtttttgatgttgtcggtgtcgtggaatggaatgccgaagcgttttccgacgtgagcgataaccgacgcggccttgagtgtgttgatgttcccaagctttaccacgttccaagtgccgtatttctgctggagatattcgaacactaaatagcgatgggtatcggcaaagtcgatatctatatcgggaagatcggaacgggaaatgtcgataaagcgctggaagagaaggcgatgcgggagcgggtcaacctcggtaatgcccaggaggtagcagaccaaagagccggctgaagagccgcgagccgggccgaccagcatatgcttcttggcgaacgcaaccagatcggcgaccaccagaaagtagctgtcgaagtctttcagctgaatctgcttgatctcttcttggaatcgatcctcatactcctgggtccattccttgatgtggccgcgactgagacggtaggcttgcccctcgcgagccagcgcgacgatatccccatccaggtggatcataggcgccttcgccagtttcacatccgccaattgttcgactaccgcacgagtattggcggcggcggcatcgaactcttcgcgagtcatgatgtggcgtaaacggctccacaactcctcttcggtggcgatgtggcgaaggccgaccgattcccgcaccttccaggccgacgcgaaatcagcatggtcgatggacggcatgtcgttgtaagaggtaattaccaccggctttccgaatgccctggccgtctccatagcgccgtgcgcggcgaccatcgacgcgggattgatgtcaatgtagtcgattccggccagatccagatgggcataggcctcgccagcgaatttgatgacgccgtcagcttcctggaattcttgcggagtcaatccttgattctggaccgacttggaagtcaagcggtagaacttctttgtgtccttggcgagcacccaggctttgagcttcagctctttctcgccatcgtcggcgcatttgatcgggatttccatgccgaatccgcgaggcagttctgccttggtagcggcttgttcccaacggacgtggccccatgttccatcatcgacgatggcgacgaatggggattcgatctctttggcgcgctcgatgatctccgggaatcgaccatatgcggcgccgtaggagtaaccagagcgaacgcggagctgagggaaagacatcatgcggcctccattgcttgatacgcccgatacactcccatgcgcttgcagacttcgtggagcagccgcacgtcgtcgagcgcccggtgtttctgtacatatgggccgcagtagtgctcataaagatgttgcagccgcatgcggtggccgaacaacggcgccgactcttccacagtacagatatcgagcgatgggaaattgacttcgtccaggccgaacttgccgcgagccagatcgcaggccagcatgaacttatcgaatggcaggttgtgggcaatattcgcgtcggctctggaaaagaagtcgcgaactttctggcgctgatcgaggaaagatgggtgcttgatcaggtcttcgttcttcaacccggtgatcttggtgatgatttcctcgataacgattccaggattgcagatgaactctacttcatccaaaatcttttcgccatcagttatcacgccggcgaattcgattattctcggctgctttctcagactcaccctttggtggaacgggagtcctgtggtctcagtatcccatacagcgaatatcatgtttgttccctcttatgtcgaaaggccggctgctttcgcgaccggcctgaggagtataccgcgacggctgaagatttacgccttctgtccgtctttcggcgtgatgcggccggagcgcatggtggcgtgaacaaacgccgaatagttgatcgagtccaaggccgaatcggcatccttgaacccgctattcgccaggcgagtgagtttacccaccatgtgcatcacgaacagggcgagtcgatgatcatcggcggtcttcgccaccaggccgttcgggaagaggatttccatgatctttccgtacatcagatcattgcgaccataggtgctctggcgggcgcggaagacttctgctgcggcgtacagattgttgagaacatcttccgcgaaatcatcgggatgggaatcatcttcgcccggccagacggattccatggcgaacggcgcggcgtcttcggtcgggtctttgggctgagccgtttcagcgagcggagacggggctttcgcaaacgcctcgtcgagggtaggggcggagttcggggcggccgggaacggctcgcctgccatgtcgttgggcgcgctatcggggtcgctatcggccgcgacggcgaagaacggcgcatcgcaaccttccaggttcaggatgtaggagtcgatccccaggctattgtaggcatcgatgatgtcctggcgatcatcgaacgccgcgacgatcttggtgacgccttcgattttcttcaggatgtcgagcgcgactgcccgcttgaactgaggcgccggctcggtgttaccatacggccgcatgatgagttcatactcgcgatgttcggcgatgccgagttcgcggtggagcttggccctggtctggaaaaagtggttgtcggttcggccggtgacgaagaaaatcatgaggtcggcgtcgatggcattcctgatgcgacctacagcgtgcgggttgagagtgtccttgtcgagacgagaatgatactcgtcccattgccgttccaaggcgaagctcttgcggtggctatcgtcgaatacgcagccgtccagatcgaagatgatgatgccattcttgggtttgcgttccatattcagatttccttgatggttgctttctgggtgacggttttatcgatgaacgtcccatcagaagtgaggcggaaaacttcgccattctggctgaaatcgaacgattgcacgttcatcgtgatgcgaatggattcgctgccgttggtcacttcgacttgtgcgatgtcgccaactttctcgacaatgatcttcatgttacatggacttcccattaacggccacagggctggcctcttgtttttccgatccccagaaatcgcggcgcagttcttcctgctcggccgagcgatccatccatggacgatagaacttgcactgatagatcggtttcaccggcaactcaaccacaggaatctggccgggtttcgtctccagagcggacatgaagcggtcataggattgctcatgaatctcatcttcattcagaaccttcgatccatagcgcgggaaggcacaggagccagtagcgacacagtgcggctggagcagactatcgaacatcggatagacttccagaaccagtcggcgcatttcgcggaaggcttcctgatactccccttgcgtacgaacacacaggcgaactttcgccatgtcgctcagagtgcgcaaattgaacttggccgcgatcttcgtttccatgttggaaggaatgatggcacgagcatcctggagcgatgcgccggcctccaagagcttctggtaactggtctgcgcgtcggcgatggcatcgtgccacaggcggttcaactcttcgcgggcgtgataggtcgggtccggctcgccattggccgtagcctgctcatcgaaatcccagcggaatgcttccggctgaacaacggcgctaacctccagggcgcgactggtttcctgctggtaagcaccggtccgcgtccggacgagttggtgggtaaaattcttgcttacgccctcgatctggaaaatgaagtccacgaattcgaacggcgagcgaatggtgtccagcatgtacttccagtggtcgagcttttcggcttcggtcatggtcgccgggtcttggccgcgcatgcgggtggatttcgtccccaggaggagttcccaggcgttctgggtgtaactgatcagagaaatcttcatcagaaatcttccggaattggcgtgaaggtgaatttttctgtgagagcaagtgccaggccttgggcttgctccttacgaagtccatactgctccatctcttccttaagcagttcgcaggcctccagacgagactcatactcttcagcctcatgaactgacgagaagacggttccatcagaggttctgtatacgagttcaatagccatgatcgcctcagtagcagcggatgatttcggcgcggatatctcgacggtcgaggtagtgctcacgaatcttatcgcgggcgcgctcggcctcttctcgactgccgaacgacaggttgaacgaagtgaaaggcttatcgtccaggcgcccatcgccaatcaggtacaggatgcctaccagcacgaaagacggcgctgtctgtcgcgtttgcggcgggtcgatttgcatttctaggaaagacatgggaacctcttcaggatggtctggtgagtacatcaatagcgctcctgctgagcagctacggtttccggctcgtagatgagcatgtcaacgatttccgggcactggcctttcacccagtcgatggccgcgaccagtgtggtggaagcggagaaagagaaagtgcggtaagactcatggatgtacggcgaccccatcgaatcgcgctcggtcgtgcgagaaatgactgctttgatattaacgatccggcccatcttcggcctccactttagcgattatatctgacaggctcagcttctcgccattcaggaaataactggcgcgaaacttcctgtcaccctctggcccgacgatccgggccgttatggtgagactcccgccgccaagggcatcggtgtccctgaagaaagccagcagcgcccgcttgagcgctgcctcgcgaggatcgacggccattaacctaccacgttccagccgtgctgagcgcaccaaaccgcaccggcccctgcaggaaggctattcagaaggaatacaggggtcaggcggccatcctcggtcatgtggatgaagtagcgtgcactttcaccgagccattcggccttggcgatggcgcgttcaagattggccttggtggcgtaggtcttggtggtgtttttgtcggtggagaaggttacttcgcgggccattttgtcgattccttttggttgaagggtttcgcgtttcgatgagggaatactactctcacctgactcagaagtaaagcacttcgtgtcgattatttcacgaacatcttcttggccttctgataggacgaagaagtcatcaggcgctcgatgacatccatgtccgaaaccaggtcatccagaaggacgttgcgccaggtagcgaaccggccgagcgagaagatgccggcttcatgggtgaggttccagatcatggactcgcgctcgtcgcggccgagcggaacgattttgcctttggtctggatggtcggctcgccgtcctcgatgagatgccttttcctgatgccgaaggccgcgcaaacgtaatccatatcccagtcgctgtcccattcgatggtttcgattttaccatctaaagtctccactatccccttagtgatggattcgacgatcagagtatcaccggtgatggacgctcgaaacgttcccactttaggaccagggaaatacacggtctggaagacatcacaagggatggaaagcttgtatcgactcacgatgatggaggttccttcaccgaatgacgggtcgattcccaggtccagccctgccgcagccaggttggcgcggaatggcgcggtgctgatgacgttcacatggtcatcttgccggcgaagatactggaagaaagaggcgtcgaaaggacggctccaagtgatacgattcgcaagcttagccaccagctgctcatagtagtcggccggtgcgatccaacgcttttcagtcgccagattccagatggaccggtccgacaggccgcccgttactttcctggagtacatgttgcagtggtcgatgcgcggctgggaaatgaactcgccgtcgatgtagatagctttgtgtacagtgacttcgcggaacgggatgccggtgagttggccaatcactggcgagcggaatcgcagaagtgcgttgtggcgctccttattctccggcgtcgccgcgtcgatgatttgggcttgaggaaagcgatgcgcggcgatcagtccagcgagtccggcacctacgatgattactttctgatcaggaatcatgatatgttccttatgagtgtacaaaacttgagaggataaaaaagggacccattttcatgagtcccttgaagagctagacgattcaggctcagaggagcggcggcttactcctcctcaccttcggaaccatcggcgccctgatcttcaccgtcgtgctcctggccttcgtcatcggccttctcgtcatcgccctggccagcttcgtcttccttcgaagcgatggcaaccagatcgacccagcccatgatttccagcttgctcagatagctgcgaaccgaggtgccgtacagcaggtgggccaccttctcgccgaaggattcgatttcgaccggctcaccaacggtgcagtgctcgttgatgtaagcaaacaccttgccgcgagtcgagaaggcctgcggggttccatggccgtcgccggtcgggatgaagtgagtggcgcgggggcgacgcgagccgttggacttcaggtcttcgcggcgggcttcggccttcgcgcggcgctcttcctgctcttccttgcggcgctgcttctcggcttcgcgctctgccttcttctgctcggccaggcgcttgcgctcttcttcgcgtgcggctttctgggcttcctgcgcggccttcttctcttcggccttcttggcgcgctcggcttccttctcggccttcttctgctcgcgctcggcttccttggccttcgccttctcggcctgctcggcttccttcgccttggccttttcagcgcgctcggcttccttggcggcggccttttccttcgccttctcggcgcgctcggcttctttcttctcgcgctcggctttgcgcttctcttcgcgctcggctttcttctgctcggcttccttggccttggcttcggccttctcggcgcgctcgcgctctttacgttggcgctcagcggccttctcggccttgcgcagggcagcggcttgttccttggtcagctcttcgccttgggtctgttcgttctggtccttctgttccatgttcttactccgggaatgtttaaagggatggcttattggcctgtgaggggattatctctaaactaattgaagaagggaatacccttagcctgaactttcctaaatattttctttcgggaaagtccaaactctagggaacttatttatgttcgagaagttcctagcttttacgcaagaacagtaagtattcgattgcgcgagttatcccagtatacatcaactgactataagggatggacggcaagttttcttctaacatggcaacccgtttccattctgaaccctgcgacttgtggaacgtcatcgcccagccgaagtcgaatccgccaatagccttctgcgcctccagccgcacgtcttcctcggccgaaaagctcaaaggattgaacttcacccagcgctcatagttcgtgccgataatgcgaactttggcgaacaacatttcgtccggctcgtcatcatcctcttggccttcggggaccggcttgagttcgagcaggatggcttgctcgccgttcataatgccatattcgtgctggttcccggtgcaaaccagcttctcgccgatttccggttgtccacccttgtagccgaggatgcggcgcgccctggcgttcaaacggcggcgagtattgttgtaggcgcaaaggatcacgccatcatcgtccaagaacgtccgcatttcgtcgtccgacatgtcgaatccggcccgtaccagaatgtcgtcatactcgcggcagggtaggcgcttgccctggcggacgaacatcgacgcccgaacgatattgccggcgttgcgctcgatttcggtcatgatggtgtcacagctgttctcatggaaaatctggacgccgcgcacaggaggaacttggccaaagtcgccaatctccagaaccggaattcggtgcgacagcaagcgctcttcatcccactcgccgatcatggacgactcgtcgagcacgaccaacttcggcttctcgtcgagcgagtctttgttggcaaacatgatttcgccgtcttcatcttcgccaatcggccgatagatgaagctgtggagggtccgggcattggtgcaacccttctcgcgaagccgcgctgctgctttcccggttggcgcgacgaagaccgtccagtccatcgagcagcaaagttcggcgatgatcttcgcgatggaggtcttaccagttccggcgaaaccagcgagtcgatagacctggcggcggtgcgctcgatcacaccaaccgcgataccagtccacaacggaattgatcgcgtcgatctgctgactgtttggtcgaaagccgaatcgctcttcgatctgatcgacggtgaagttagatgctgacatatttgcgttctccaacgctaggtttaattgaatcgaggctcagtttaagcacgccgtccacagaccagccagtatcacgacgatacttgcggccttgcggatcgacatagaagttcttcgtgcgccgcagcagaacataatgccaagcagctccaagcgcgtggacccttcctttatacgggaaagccttcgcctgctctgcggcctctttggccccagggagccagcggacggtagaaaggaccaggaccgcacccttaatagcctgggaagcggcgcggccgtcctgtgggctatagcgatgtctatcgaagtctacccagtggtggttgccgcgccggagcttgaccgttcgggatcgcccctcgaacaccaccgtgccttcgtgagtaaaaatatgttccgccatcgaatgttcctttataacgtacagttatgctttacctctgcgcaggaagagtatactatcagctgactcgccaaagcgagcgaatttaatccaactttacttcggcaggaaagtggccgatactagcgccgccgcctgtactgccctccaaaacagaggatacattaaatgcaagaatgcaagatttcccgcgaccaactcccggtcggtaatccgaatcccaatgtcgacaagacccgcgacccgaacctgaagcccggtttcctgcgtcgcagccgcgagctggaaccggcgctggcagttcgcattcgtcgcgagctgatccacgccgaagcgtccgacttggccaaggccggatgggtcaattcacagtccagcctctatggatcgaaagccttcccgcgccattccgtcgttcgcgttaccggagttccggaagatggagctttcatcggcatgctgatcggcttcatcgagcatcgcgagcatggcgaatgggcggtcatggaagccggaacgaaagaaggcggcgcagtcatcatcccggtcaatcacatcacgcgagcgtcattcgccgaagccgaagagttcgccgaaaagtgggagcggaacttgggctggcgcctcctgcgacagctccgcgagtgcggcgccctggccgggactgaagacgagttcctgcggcggatcatcaatcgatacgttcgtgatcgcacgatcctcgatcaccacaaagtcggcgcggacaaaatctatactgatgcagtactcaaaagcatcggcgaaacatggccgaagattccttcggggaaattcgtcggacaccgagtcgcgcagctcctgatcggccacaagctaggtcgagcggggaccattctgaatgacctggtggacttcctggagaagttcgcggccgggcgcgataaaattctcaacatcgccatctgtaattgaggtgaatgaaatggttgacttggaaaagctgagtccaaagctgagtcctcggcagcatgccgttctactggagatgtacaggcatcctgagcatccagttcacagatcgccaacgaatttaactcttaaggctttgagacgaagggggcttgtcgagctatcctggaaggattccgtggctggatatatgttcggtcgccaaccttgctggaaaataactgatgccggcataaggcgaatcctgggagtgctggggaataatatggaaaaagaacaaagcgaagatcgcccaagcatccctgagtttgagaagaccgacgccaacaaactctatgatgctctggagacgatttcgctgctggagaagaagttggacgaatccgaaaagcgcgggagcgagctggccgcgagctattgtgacggcgtagtcggcgatgaatacggccatccttattgccgttataaagtggaacgcgacgccgccttggccgaagtagagcgcctgcgagaatcaaaaggcgatccctctggcagcttcgacaggtgtatgaagatgatgtacgagcgcgacgagaatgcaaaacgactcgacgccgccttggccagggtcgcggagctggaagggaagttgacggactgggtgcacgaagggttccggctcaacgaggcgctagcggtcgccaaggctcaacccagcgtgccggaaatatctggcatcggtcgcgatgccgaacatcccagagctgtagtgctgtatctgcgtaacgaacccagcgatgaagatatgcgagcaattcagaactttttgcgcgccatggccgcccccgctcagcacagcgtgccgaaagcatggctcgacgttcaggcagagcgccgccggcaggtcgaggcccaggcgctaaggcaggaagtcgcagcactgcgcgcaagggcggatgttgtgccggaggtattcgggcttgagcggtaccgcgtagaaaaaactgggaagggtttctggccatactgcgtgcgtgcgggggatggaacgcgtgaactgttcgtcggccacttgaagcagtgtcagcgggtagcgaccgaattggctacggcgtttgaagatgggaagtttatcgccgcgcccggcagctcggctcagcacagcgtgccggaggggtggaagctcgtaccgatggacccgacatcgcagatgacctatgtcggtcagtccctgcgttatgacgcggtaaacagcatcggcgagatttaccggcaaatgctcgccgtagctccctcacccatcgatccggccgcgcatcctcagccgtgccagcaaccacaggcccatcctgcccgctgcgggtgcgagcggtaagtgccaagtaaggaattcatgtaatggaacagaagaaaccttcaccagtagatggagtcatcatgaccagcctcgacgttctccggaaagcacagcccgaagcccaggacgagtatgccgtgtccatgttcgcaacggcgatccgccagaaaatgcagcgctcccgcgataaaggccgaggcggctggatcgactgcgacgaagatattctgatcaacggattcgccgaacatgcgctgaagggaaatgagaacaacctcttggacctggcgacgttcctgatgttcatgtgggtgcgcggcatcgacgatgcgaagattcccccggcgctcgaaaaggcgcggcagcacaagatcatggaagcatggagtcgggtccacgaagatggactaaactccgccagaaaggcgagtgctgcgcgacagttcgtggaagtgcctcgacgcaaggggcgcccggagcgacttgcatgaagcctcacgaaataagactggcacaggccgaagagttcctgcgcgaactcggccgagggattccggacgacgaacgggtgatggtcggctacgctgaagaggccacagtccaaaccgacgagaacggtcgcaagctcaacgccggctggtggcctgtgccctggaaggaagggaaatacatcaactcccgatccaacgcctacgcctgtatatcgtcgtccatcaagacgcccaacccgaagaccggccagatgcgatactggcgcggcgaagcctctttcggccacggcctagcgttgatggttgatgacatcggctccggcaaagggtcaaagggcgacttcgaccgcgacgagttccgcgagcgacttgagccgaccgcgattgtggagacttcgccgaacaactaccagttctggtatttcttcaaagagccgatgtcccacatgctccagttcaaggcgctgctctattcgttcgtggaccaggtgctaaagaaaggcggcgacaacaccgtcaaggacgtcagccgttacggccggatgcccttcggcttcaacaacaagcgcgggaaagacggcaagttcaaatatgccgacgaaaacggcaagcccgaactcgtgcgactgttcagcgccgactattccaagcgctactcgccagaagagatcgcccaggcattcggcgtccgcatcatcatgccacagatgaagaaggtggagataaaccgcgacgattgggtgtatgaccaagtatggctgaagtatgccgagcacatctgcacgaaatacaagatgggcgaagcggcaggcggccaggtccagcagaatatgtccggcaaataccgcatccgctgcccgtggggagacgagcacaccaacggcgatccattcggcgcctactttcgcggaccgatccctggagccgagcacgaatatgtgttcggctgcggccacgatacttgccgcaaagagcatcgccggacgtgggcggccttcacggatgaagtcgtgctaccctatatcgtcgaacaattggaaagaatcaaccgccgtcatatcggtgaggagtagacaatatgcaaaacgatcctggaatcctgattaccgccatcggcttgctgttcctcggccttatcatcttcttcgaaggcctaaagggatggaaaatacaagtcgcaaacttcctcgcgtcgcttctgtgcttcttcttcggcctttctgctttgacgttctggttcgtcgttgcgtttgacgtattttaatcgacgaacggtacagaaattttcggatggggacggaacttattagctatgccggtttaggtaggagataatagccgtccctttcgcctcaatgtgtagaggcaatgttgaatccgatcatgtaaagcagaaggcggcaaacctaacatgattatcgacgaagataatatttttgatgatggcgaatcagggtccagtgagtttgatctcacgcagatagaagatgctggaatggaccctttgatgaccgccgcgagcaaggcggccgatgatgcgattgcgaggaacgaaacgcatcgcgcgcaaaaggcggcaagatacgccgaggcgtatgctgaaccagacttgaggaagcgagcgcggttgttgatgctcgaccaggcgttcgatcttccggtcagccgggtggtgaaagggccgttcgatgacttcatcacgaaatacagctcgacgtcagacagcaactacctcgcggtctatgatacgttgttctgtaagggcgacggaaccgtcccacacccgcacttcgacgagtttcgcggccggctggtggaccatcgcggcgtggcgttcaacaacaagaccctcgatccgattgacctgatgggcgccctcgcggctgcggccttggacgatccctcgattaagaagacgattgagacttgctgcgtgtgggctcgtcgatatcgccgcaactctctgatcgagacgttcgagaagaagataccggagtgggatggtgaagagcgaatcgacacgctgctgatcgacttgttcaaaccgttcgacaccgaactcaaccggatggtgagcaagtatttctggctgagcttgtactgccgcatcaactaccctggaatctcggcgccgatctcgttggcgttgattggtgggcaggatgcggggaaatcatatttcggcttgctgatctgtaaggaactgtccggtgggcgcgatctggctccagttcagctcgacctgagccgacacgaccagacgccgttcctgcgcaacatcaccggcaactcggttattgcgaacgtcggcgaaatgtccggcttcaaaaagggcgacatggaacgcatcaaagaattcctggtgcgatcttcggatacattcgatcagaagtttgagcctggcgaaacgatcaagcgacaatggatcaccatcatggacggcaacggatacgatggactccagcgggacgactccggcaaccgtcgattctatccgatgttcgtggcacaacttcccgacgaagatggaaagccgaactgggttaagccgggcgatggtaatgaaccgttcaaggtggacttcacagacttcggccgcaagttctggcaggctatggctgagtgccgcgcctggatcgaagagcatggcgtcgatggatacctgaatatggtgtcggaagctaaccgcgaagtgcagaacttctctatttcggaaatggagaatgcgcgtggcgtggttcgtgatgatacgattgacatgtatctgatcaacgttctgataagttgcgagttcgaagagatcaagccgggcaagaattccaagaatcctgggtggagggctgacaccgtcaccattctgaaatggttcgatattctcgccaggaagaagccgatttctcgccacttaactccgcacctgaaggcgctggggttcgtcccgaacaagaacggcctggctggttggtgcctgcccgtcgataaggttgcgcctggatggacgaatagtatgcaaaccacgctgccgccgttcaatgatgcgcttgtgtacttgctgaggaagggcgacccggatatcactgatgaggctgcaatggcaaagattcgggcagtccgggccgagcgagccaagattttgggcgaggatttctgatgtagtggtggagtgtacttggattaggccgccttcgggcggtctttttctttggtgcggcgtacattagattccggcgttgggcgggattggtagaagtgaatacagtaatttggaccagtttcgagcgcgaataatggaaagtcgatagattttcggtatcggtttagcgtgtatggtggatggattttgcgtgaaatgttgagaaattgcgtgtttgaggtggacttttcgtgggaaatagccgcaaattactgtatagcgattctgagcggtaatttgggaagcctactgccgcgtgggttagcgggccaatttcctaatttccggtttttcgagcatgggttaaaactattctacagcgaaaat

SEQ ID NO: 2 - DNA, 65924 bp, synthetic construct
  misc_feature 1..65924: note = Nucleic Acid Sequence of ECACC 17062006 Pa204
  source 1..65924: mol_type = other DNA
cgattgcacaatcctaatagaaaaaatctatcacggacgttacctatctttaaaattaataaaattaatagtaatttggtaatttggtaaactttattatttgaaagcctcgcggcactaagcttgtacatatcccgtcaaatttccgattccagccagctcgcggcagggtcgccggaaacttccggacttccaatccatgggtcgcggcaacaccacggcggactaagcggcaggcaccaaaactcgacgaacggaaccggaaatttgggagcgcgacagaatcgctcagctggacatatttctaacattcgatttaacattcgatccaaacactcatcgccatcgtcgcccgccaaccgccgactcgatcctcacccaccagcggatcgcccgtataacatcctataacaccacctaacactcattcatcatcaaacccacccagacctacatcccacccacaagcagcccatagacgcgatccctggccccatagtacaatcgcgccatactcagtgtcgcggcagagcaccaggcccatccaccaccaacgccaccgcgacgactccagaatcgaactcagggacgcaacaccaaatgaccaaacactacagccccgacgatctagtcacgccacaggaattcgcagacccgcatttcgcagcgatcaaccaaaagcgattcgacctgtatatcgacctgcgcgtccaaggctacagctcctggcgggtcttccgggccatctggggtgaggagcacatggacggtcccgcccaggctcgcatcttcgcgatggagtccaacccgtactatcgcaagcagttcaaggccaagttgaacgcgaccaaaacatccgacttgtggaatccaaagacggcgctccacgaactcctccaaatggttcgtgacccaaccgtcaaggactccagccgtctgtcggccatcaaggaattgaacgttctggctgaaatcacgttcgtggacgagtctggcaagaccaggatcggtcgcggtctggcagacttctacgcatcagaagccgaggctcagaccgccaccgtcgctgctgcggccgaagccaatggctatgtgccggaaggcgaagagggcgatttcccgtccccgacgccggaaccgaccgaggaagaccgcgccaaccccatttagacataaaataacatcgttctaggcccgaatcggatcgaactaaggcgacggtagcgggaagggacgaaaaacgattctagggcggttctaggaagtcggagcctaacctcagaaacgacaaagccccggactctagctcagaatccggggctttcttttgggcgccttattcgccagcttcgatgatttcgaagttgtatttgacgccttcgtgctcgaaagtcatcttgccagccgccttcagctgcatgcggaagcggatgtgtttcgaagaaggcaggccgaactcgatgaatgctgcgttggtggaccggaactcgccgcgcttgcctttgacagtcacagccacgccatggcgctgagtgcgtttcttggaaacttccgggtctttccaggagttggcgatggctgccgacaggtctttggactctttggctttctccggagcgttcttggcctcttcgcgcatcttccggatttcttccagcgcttcctcttcggtgatctcttcttcaggcttcagattctcttcctcggccttctcttcttccttcttcttggaagtgcgggttttgtaaaccttggccggggcttcctcttcctggaaggcctcttcctcggccggcagagcgttcaggatggcgaggcagcgacgctcagcggtcttgcggtcagagaagcgcttgacagtcgcatcggcgttgtgagcgttgtagaaggcgaccagttctttcatttctgcgttctggatgtcgccgaaggttttgatggagttggtcatcttgcgatcctctgttttggaagatttcttttgggcttcggtttgtcgccccgttgaaagagattatgcctaggtcgatgctgcgtgtctacatctatttcatcagattgatcgcaaagccgacgaacggtcgtcggatgtgaaaacaccgcagaacaggctgcggtgtttggtgggcgagggcgatggtcagaaagtcgggactgtgatcggctcgattggcgggtcgccgggcttgtcgttgtccgatgcgggagctggactggacgggatcgcgccggaccgaagcgacagagattccgactgcctgctcacctgcggagcttgaggcgcttttggcgcatgaggcgcgggcggaaccggcaggcctggaggagccatcggcgccctgggcggctccggaagatcgggcagatcaggaagactcggcgctatgctgccaaaggaccgctgcgactgctgcgccggattgtcgccgaacatttcggcggatcggttgtccttgatgacttgacctatggccgtcttgtcgccgcccggctctgccggatcgaagcccgtgaatggagtggtaacgcagcgcatgttaggatgggtaggcagatttttcaggactatggaatctgcccagacatacggattcttgtggttgccgggtccgtggctgatgatctcggcaggaatatggccgttcatggtaagccattccgccatcccttgccgagtcagcgggcttaccacgatctggccagtggactctttggctatggcgaacagatcaccgaattcgaatccgatgattgtgtagttggacctggactcgccgacgaaagccagggtttcgatgttgtggtggatatcttgatcttcccagatgtacagcatggtgtcactcctctttggcgaaattgttctttcgaaggtagtaggcgatcatccgtcacgacaacgtggccagaacggcgtcagtgccacgttgtcgtgacggatgatcgcaaagttgtccgttccttccagccctgcgatgaagcagcgttcccaggcgctcccgctgtagcgatcatctggcttccagacgttcattcctgggctgagttcgctcgactcccatttatgatcgatcttgacgttcctcatgacgccctcaataaggctgcttgttgtgttcgaccagagacataccggccggaactttgaagcgggcgtattcaatcatcagcgccgtgcgacgccgattcgtgccagactcgtcattaacgaagagttcgcactgatgcgggatgactcgcgtcaccaggccttcgcctttcgagccgtagaacttgatgtagaccgcgacgcctcgcgtcatgttcagatggatcgtttcgcagagtcgctgcgcggccgacgcgctggacagatctgtcggactgaccgtcacccagtaatggccactttgttctttatcggacataaactgccctccaatgagaaagcctctgccgagccgcagaggctggttgttgttagtcgcgcttcaacagaacgactttgtcatatgcgcgatacttcccgcgccagtcttcccagtattcgctggccgggcagacaagctccagagtgatttcgtcgcgccagcattcgacagaaagtacaatagccttgttcctggctgcggcatctgctctgagcttaatcagaatttcatcgccagttttcagttcatctacgcgaacaactttagccatgacacactcctgtttgaagaggcgcggccaggacgatcccagccgcgccgatggattaacgtttgtgaaggatggacactgcatccacgtcgaggatgctgatggtacggcgacggcgcggattgctgcgctcatggatgcgaatcaggttcgcaactggagcttcgtccaccacccatacatcttccggcttctgcgtccggagcaggaccgtcacttctgtattctgggcgaggccgttgcaaatgaacgtgaatttggacgagggagttctgtacatttcgagattcctttttggacttcgggtccggctactcagccggtgaagagattatgcctgatcatcgccatcgagtaaagcacttgtgtaccaattctccagttaaatggaaccaaagcgcggtatcgctgatcgctacgcttccgcgccatggcccttcttgctcacagacttcgaaccacaagctgatttccatcctggccaggactcggccgaaggattcgagccggcgccggctagccaggatgtcgacattcacccgccgattgatttcgtaccggcgaccgttgatgacgaagaccaggcgcagggtgcgtccgtccagacattcccagtatttggtttcataccgaagccagtagcgcttgccagccgggcctaccatggtcatttaatcgatgctcctggccgcgccacgcggtccggtcggcgggacgggatcgaaccgacccaggatgtagtcgggccggcgcgcttcctgaggacacacggcgagaaggcgccatcctgcgtccagggcatgctggagttcgtccgtgcagcagtcttccttgagcagcagacggttgatgctctggagattcggaccagggatggccgagctggtgtgcgagttccagccttcgatgccgtgcaccatctgcggctggtcgatgtagccttcggacctgccggccaaccggctcgcggccagctccaggcgctccagcatgggtcgcagagcggcttccgggtcaacatcgtcccagagaaggaccaagctgatgatggtgtacggatagtccttgtccagatcccaggccgaagcggtcagacggcccaggccgatttcattacacatcacgggaacgtcgttgctccatgtggacggctcccagttccgctcaccaggattcccgattgttacgccttccaggttccccaggagaacgtggagcttgctgacatattccgcctccaaggctttccgctcttcgtcggtctggttgtggcgatagaaggatggagggctgacttttgcatggtagagtttcataactgttcctcggttttggaagatttgaaagttagagaatggtgtcgcagtatttctcgaaaggactctggcgcttcttctcgcagatcgcgcaggtgatttccaggtcgggcagctcgctgtaagtcttgccgagatacagccagcgcttgcacagactgcggccgtctgattcgaagaagtggactttgcgagcattgccgggttgcgcccagccaccttgatcgtttttgcgcttgctcatggcgacatatcctctggatcggggatccatggtgtacgttcagccacccaaactccatctacatagatcaggactgacagagcagtcgcgccaaacccgaagcctacatagaatgaaccgggatcgatttccatggtggcgctattggtggcgctattgcaatcgacttcaaagtcgaatagcgaatggccactcataccatcaccatgtcgatttcgctggcgaagaagttaacgccgactccatctgcgtagccggtgtaacgcagccgaatagtaccatcgatgtgggcttcttcgacgccgatgacttccaagatggtttcgtcgcagtgcagcttgacgaacatcttgaagccggagccaatcgccgcgaaggcgatctgcttgtacaggtcttgcttgatcatgctttacgctcctgtttgcggatgtactctgccctggcttcggcttcgagcttcgcccaaagcgcgttgtcgactggaccccagggaccggtctgagttccgtccaccggtgcgaacttgccaccggtgcgacgatacttggacagagttctcaggctgatgattttctcttcttgtgaatcgagttggaagatcacttgacatacttcccgaagtagcaggtgtggaggatttccaggcctttggagatagccttcagcttgatgccgaaattcttgatcgcccagtccagatcgtggtgatagaactcgatgatgttgacgatttgaccggcgctgtagtggatgacgcgaatcttgccattccagcggaaatcttcgcggctgtttacggcctcgactgcgatggtgtcgtttctgccgatggtccattttgagctagtcatgtcgcaatcctctttttggagtgtttcgcgtttcgatgaggtgactatacctcagtcacatcatcgagtaaagcacttttgaacagattgtctaaaatttctggaagacaaaatcaatcggccgcgtcatcgatagcgaacgccgatcgcgccaggtcgatgatctctggccatgtcaaagtaaaggttttgccgtcatcacgaatgataattggagtccgcagcggctcgcccaccagcattgtgtaggtatgtttctcgccggatgcggagccgatgcgggtgcttaaggtgagcggggattctttgtgtacggtgccgatcattgtttcgttcctcggtggtacccggagaatcgggcgcgttggattaggctttgccgcgacggattgttacgccgcattcagtagtcacttccttagcggcaggatcgcgaacaagataagcacaggcggctttgacattcaaagctcgggttgcgccgagtttgcaggaaagatcattcccctcttcgtcccacagatgggcaatgcgagagatacagcccttttctacttcagcttctacagttacggactggtcaatgttaaataccggcatcgctttctctccgagattgttccgtttcaatgaggtgactatacctaagtcacctcattgagtaaagcattttcttaagattatctgaaaccttttgaagtcaggaactgccgccagagccagtcgatgtgttcgttgcagtaacgctcgccatcggacgacatagccagagcaccatccttgatgccgaaccgggtaacggcgagccattcgaattcgaactggagcgttcctgggcgagggatatgggacatggcgtatttctgcacggtcagtgatcctcagaaggtttcgcgtttgcgatagccgctactttctgcataccgaggcgatagaaatcggcttctttcctggctttggtaagctgctccatggcgtcgcacgccgtcttgtgctgggaattggcgttcttgatgctgccgtccaggctcttgtctaactcttcccgaagagccttctgcgatgctagctcggcctccaaatcctggattctgagttgaagctggcggttctgctcggcgaccttgtcttcgcgggctacgaagttctgcaggcggtcgacaagacgagtattttcgcgcaccagctgctgtttggcgactttggtccttcgcagttctgctcgcaatgcggacaactccagcgcatgcagatcggcgtccgattcttgcgactctgcggcctgcttattgcgcagatagtattgcccagccgcgaaggcgagcagtgctcggtctgtatccttaactgtcttggccatgaagtgggctcgatacagaggcgcgaacttgcaggcgtctgggtcccaggccagattatcggccacaggccagaatgcgcgaaagcttttcgcatcgacatgcgccttgaccgtctgcgatgcgcccgactcttcgcagatgatttccagagggacataaatggcgttcttgatgcttcggaaagaaatggtggacataacacaatcctcataagaagtcgcggccgggaccatcctggccgcgcatcgtaatcactcttcgccttcgtccgcgctcagccactcttcgaaggcgaaattcaccttcgactcgacccaatcctgcagctcatcggcgaactcgtcactgtcgatgtccatggtgattcccataacttccatgttccatagcgcggcattcagttcgaactgtaccatctgctcgccatcgacttccagcaggatgcggtctgcgacttcatagtcgtctacatcgaacagatggccgcccgccaggaggtgctgtacgaaagcttcatcgaggttggtgacttcgatctggacttgtttggtcattttcgtatccttacttagcggctttgaattgggctttgaggttggccagttcagctttcagatcgatcatcgcctgaccgcgcaggccttccatttcagcctcgctgattgcgatcttggctgcgcggattttgtcgttgatttgatctttggtcatttcggcttccttttgtttgaagggtttcgcgtttcgatgaggagattatgccgacagtcagactggaagtaaagtgtctgatcgaagaaatttctgtacaccgacgaacggttgtgctcgaccgtctgctgcggtgtcgatatactcggcctattgcgaacaatggactgattaaatgtacaaactcaatcctgcgctgcgagcggtctggcgaactcgcgcccgttacaaagtcatctatggaggccgggcgtcttcgaagtcgcacgacgctggcggtatagccgttttcctcgcggccaactacaagctcaaattcctctgtgctcgccagttccagaaccgcatcagcgaatctgtctacacattgatcaaagacaagatcgaaaactcagagtataatggcgaattcatcttcaccaagaactccatcaagcacaagattaccggctcagagttcctgttttatgggatcgcccgtaacctgtcggaaatcaagtccaccgaaggcatcgacattctctggcttgaggaagctcactacctgacacaggagcagtgggaagttatcgagccgaccatccggaaagaaaactctgaaatctggatcatcttcaaccctaacgaagtcaccgatttcgtgtatcagaacttcgtggtcaaaccaccgaaagactcctgcgtcaagatgatcaactggaacgaaaatccgttcctaagcgagacgatgctcaaagtgattcacgaagcctatgaacgcgaccgggagcaggccgagcacatttatggcgggattccgaagactggaggcgacaaatccgtcatcaacctcaagttcattctcgcggccatcgacgcccacaagaaactcggctgggagccggccggatcgaagcgcatcggcttcgacgttgcggacgacggcgaggatgcgaacgccactacgctcatgcacggcaacgtcatcatggaagtggacgaatgggacggcctggaagatgaactgctcaaatcgtccagtcgcgtttacaacctggcgaagatgaaaggcgcatcggtcacttatgactccatcggcgtcggcgctcatgtcggttcgaagttcgccgagttgaacgatgccagcccagacttcaaactgatctatgacccattcaacgcgggcggcgctgtcgataagcctgatgacgtctacatgaagctgccgcacacgacgatcaagaacaaagaccacttcagcaacattaaggcgcagaagtgggaagaagtcgcgacccgattccggaagacttatgaagcggttgagcatggaaaggtttatccatttgacgaattgatttcgatcaactctgaaacgattcacccggacaaactaaatcaactgtgtatcgaactttcgtcaccgcgcaaagacctggacatgaacggccggttcaaagtcgagtccaagaaggacatgcgcgagaaacgcaagatcaagtcgccgaacatcgctgattcggttattatgtcggccattctgccgatccggaagcccaaaggcttcttcgacttctaaacatggaaaagcccggagcgatccgggcttcgggtcttactcggtgtggttcctggcgctgagtgtcgatgcaacggcctcgccgactcccagggcttcctggccggccgcgagcgcttcggcttccgactcgacgatgaagtcatcaccttggccgtcgccgggcggaacctcgaccagcacggcttcttcgccctcgaaacgcagatcataggtcttttcgacggacagaccgtaacgggcgttgagcgcatcccagagctgagcttcatagttgcgcaggtcttgcagagatttctggtggctgagcatcgccatatcgacggcccgttgcagggtttcgtccaggacgttgaatcgcatgcgaagagaacgaatccgctcgaccacttccgcatccaccacatgtctttcgatcatcacttttcacctttgctgaatgtaacgttgtagccgttgtcggccaggtaggtcagggcgccttcgaaggaagttccgacgaggtgcttgagcttcatttcgcgttgcgcggccaaccagaatgcagttccggaaaactctgcgcggccttcggacaaaaccctgcaatcaggcccggcgatccgtacatggacggaagatagcttgatcggcatcagtgaatacctccgctggcttgcgacggcatgctttcggcgcgagcggcttcgcagtccgggcagggacaggcttggcggacgcgctccaactcgtcggcatccatgacatagagcttcccgtcggcagtgtcatgtgccatagcgatgttcgggaagtcggttgcgctcaggccggcgacagtgcggatttcgtcccagagcaccagatgctcggcattcagtcgggcggcgagcgcttcgtgctctttagcgacgcgcgccatgaactcgtccatccggaatgcgaattccgcatcgatggcacgggccgaggccatggagctgagtcgaatcggttctttcttcatgatgattctcttttggttggtggttgttagctgctcaggcctattcaaggccggctcaacgcggcgaagaacaacacgatgaaaaggagcgaaaagaatgcgaacgccagagatattccggaaatcagctttgctatgccctgcatctcagttgccttggcagcgagtctggccttagcctcgataacttccggtcgtggcgactgaacatattctaaggcctcttcccgcgctttttcggcctcgaccaacctagggtcgcgggccgagacttcgctgtgccctggcctcgcgggatgggcttgcagcgatggaggaagttcagcagccacgactccatagtcggcgcaggcccaagcgatcccgatgaggatcgcgaggatggactggacgattcgcagcatcactttgtcgctagggaagttcacgatcaatcctccaccgaccgaacgatttccatgttgcgaccggcattggtcccggcattgaaggcgcgccgaccgtcgctatcgtccagacgcatgagcttagtaacattcgatttcttgtagccaggatcgccgaaatgttcgtgaaccgcagcttccttcaccacaaccagagacgttccggcagaagataccagctccatgcgcttcctggtgatggattgaaggcgatagctgatttcctgggtcgcggccagcttgaattgcgcggcaacctttacgttgaaacgttcgtacccttgagccttctgatactcccggcacagacgatcaatggcctcgaccagggagttgaacatgttcaccgccagctcaacgtccgacttgtagcctttaaagcgtacggcatgaccccagcgcttggtagtgcttccgtcgcgggcgcttctggatgccttggccgatgctcgatggttgttgatgccaccggcgaaatccatgatgcagtcattgtacgtcgccacggccaccgagaagaacttcatccagttcgggattgcggaatagtaacgagtcgcaatctgctcgtcgaactcttcacgaatctcgccggtcgcttcgaagccgtggaggtcatacttgtccttcagcttcttcacgcgctcggctgcgatggccgcttcgtgcgggctggaggagtcggctgccatggcggtcagtttgcggatgcggtctttcgccttctcgatggcttccggggtgaattcgttctggtcggtcatggtcggttccttttgtctgaagggtttcgcatttcaatggagctattctgccttcattcagaatggaagtaaagcactttcttccactatttcggcatcgactggaagaaattccagatccaatcacctgctaccagcaagaggatgagggcggcaaagaacaagacagcggccgcgagctgtgcgcccggcttcagtttgggatggctgagcttgggctctacgggaacggccggagcgctagcgctgcaaccggcgtctcctgggccgtagccgatgccgcgttcacacgcctggagcgacaccagggatttaagatactcggtctgcttttgcgactcttcgtagatgccggcgacggcgaaccagagggcgaacaccacggacgtacacacaacccaggcgccggtcagcaggagggccagcgggccgatgacgaagacggtagcagccaggacgattgcgccgccccagatgatgaagccggccagaccattggtgatgtcgacacagaactttttcattttcagattccttcggttacgggatggaggggatttgaaactctgcgccgccgagaacatcaatgacgacttcccagagcgtcggaacagaccactggtaacggtcgaagtcggtttcaggatcgacgcccagcgtcacataggaagtggcaggccatttatgaatactgcctttccgcatgagagtcgccacgcggccgtcaccgagcggattcgtcctgtgcggaacgtacgcagacatcgaatattgctgcccggcgtcgacaacaaatctttgcctctggcgcagacatacagagcccacaggattccagccgcgaccgccgcaagcctggcgagccgaagaatgctcataaccttcgtgagtcatttcgccgaggcaagccgagcgatcctcgtaatagttgatgttctccatcgatccaaccagcaccagagactcgaaatcgaatcgatgatcgtggatggccgagtgattgaagcaaagccgacgcggcagctccgggtgccaaacatggaggcgaccggccggaagttggacctgaatgaaccccaggccgtgcagagtgatcttgtccttcatcgggtcatggacggtgctcatggataatcctcagtagcagaaatgtattgtgagagttacgattgacaagccggtcgcccatagaagggcgaaccaggccatggctttgatggtcgtgtagatcatccgaagaattttccggcacagatcgggccaatgcccatttcgatggatgcgtggttggtcaactcgcgaccgcagcaggagcactgaccagtcttccgaccgtaggcgactgccgattccatcggcttttcgaacatcttcagaacgtcatcgtgctcagtatcggtgcagtcgcgactcttgatgaatttgccattggtgatccggccgaggtagatgtcgcccaggacgtacaggctaccggcgttccggctgtgagcgctagcctctttcacaacaacgatgagcggctcctcgccttcgccagctaggcggattttcgggcgcttgataccagactctttcgccttctcaaacgctttctcgatgccggaaatgtccagagtcggcgcagcagcttcctgcgcggccactttctcgcgatacttggcgaggttttcgatggcgcgcttcgcagcagcgatctgattttctgtcaaggagccgtatttatacaacgactcctgaaggctctgagcgaagctgaaggaatttccagtccaccactcgatgatgtccgggtgcgcggcttcgaaggccttaattttgaggtcgcgctcttgcgcagcgctacagattttctcgatgcgcttttctgccgccttagcacggctcttggcgcgctgctccgggctggttttgtactctttgtatccgacgccgccgcaggcaaagcaggcgcgaccataagacgaagggccacggtacaggccggtgcctgcgcatttggtgcacttgtcgcgatacagcttcacttccttccgggagttcgggcgggcgcccatggacacgtcttccagggtcttcggcgcttcgttgttgatctctacggtagcgaagtcatcacccaggtcttcgaagccggtgaacagattctctgctgcgttcatgtcgattctcctgtttggaaagttcgtttcgatgagttgactatacgccagaaatggaaaaacggtagcgatttctcactaccgttcgtcgggtcgaagacaatcaataaatgtcgctattgatcttgaacccatgctcagcgccgtcgttgtagtcgtactcaacataactgtcgcagtagtcgttcaaatgccgaatgatgcctagaacgtcgttcgcggccttgtgccttttcgcggcgatggtcttggcgatgctgtcgccgacttccagggtttcggccgtccggcgatggatcagcaggcgactccaaagatagagccggacgcggcgaatgatgtcgcgatggcgctccagtcgtccgtgcaactcttcaatttcgtgctcgcgagattttaatactcgtcgaagctgttgaacttccaactccaaatcggccttagtagccatgttcacctcagaaaggaaaatcgtctgagactccaggaagctcgacgattattgttgagcctgatcggtctagaatgcatccgaccgagccagccctgtgtggatagggtctacacgaactataacaggtgacttccctgactattcgccaggtcgatcctccacaccatctgcaaattggcgggcgcttcgacttcagttttcgttcgtccaggacggcgcgtctgtcgcaggaaaggcagcgtgccttaaccgacgtaaccatgggtatagtcgatcattcggatgagctcatcatcctctgattctctcgacttcagaccgcccaaagagtccgactggccgtcgcatttcgcatcgatatccatcgaagacaggaatcctgccgatactgcgatgatatcaactttgtcgccggccttcttgaagccttcgcactcagcatcgagggccacaattgcactcgcggccatgttcacatgactgatgaggtcggggaagatcacaggaacttcacgcgacatgccacggaccgtcagcttcaggactacatacttcatactcactatcccttttgtatgtgaggaaagaacttgctgttttccggatggtgaaaacgctcggtcgcaggcggtctttcttccggacattgaatcgtcgaaggcgggaaaaccgcgccggcgataatcgccgcgaggagtgcagcggatgtcgacatccagagggcggtttccaggctcacccgaacttccggacggcgcggcttcattcgctccaccctcttcccggttcataggatggtagactgccgccgcgaacccagttgtcccacatatggttcagaccagtcggtggctggggtggcggactgtagaatccgggagtctcagtgctgctcaggaaataaggcgtgcaaacggcctggacgaccaattggtgtcgctcccacatcttatcaatcgctcttagcatgacgtcttcgcactgagccttactgtcgaaccgtctgctagtatggtccggcatctggacgcagccgtctccagtgcaaaggaaagcagtagcaatccatacggtgatactcgccatttcgtcaccctctttagttgatgagcagagtctattccatctgctcgccaggagtaaagcgcttttcgtcgggataaatgccgatgatgtctgcgtcgagcatccaaatctccacggacggatcgtcgctgctgatgtggtacaggttgtacagctcccgtccgccgacagcgcgctcgcctcgcggctcaaccgccagaaccttgccgtgcccttctccgtgctcgtccaggtatatgacgtgatcgccgacttcatagctttctttcgtgacgaggcgtgaacgcgagctgttctgcgattccaccacccaggaatccagcacgccgcctttcatgtcgcgaaacaatcttgccgatttgtcgcaatcgaatacgcccagaacttctccgtctttcaacacgatatgtacgattggcaaaagagtattcatgtttaatctccattggttgataattagagtctaatctgccgaaaagttcccgtaaagaattattttctcataactgattagttgcaactgttaatctgatgtatatgtttgaatctcttttgaacgtttgatgtttcccctataataagcgcacacagccaacaaccacgtggaactacaatgtttaaactttcctggatattcgggcgcaaaaaggataatgctgcctgttctgaatcggcgccggagaaagtcgcacaaatccctcaacacgatccgctcgaccccatgatcaagctgggaaggattcgcggctggaacgtcgagccggagaaagccccggtcattcgtagtgtgaaagatttcctggaaccgggcctatctgtcgcaatggatagtgcgtatggtgatggaccaaccccggccgcgaaggctgctgcgggcggccagaatccctatgtcgtcccgactatgttgcaggactggtacaattcccaagggttcatcggataccaagcttgcgcaatcatttcccaacactggttggtggacaaagcttgttccatgtctggggaagatgcagcacggaacggatgggaactcaaatcggacggcaggaagctatccgatgaacaaagcgcgctgatcgcccggcgcgacatggagtttcgcgtcaaagacaaccttgttgaactcaacagattcaagaacgttttcggcgttcgcatcgcgctgttcgttgtggagtctgacgatccggactactacgagaaaccgttcaatccggatggagtcacacccggctcctacaagggaatctcccagattgatccatattgggcaatgccgcagctcactgccggctcgacggccgatccgtcttccgaacatttctatgagccggatttctggattatcagcggtaaaaaatatcaccgcagccacctagtggtggttcgtggaccgcagccgccagatatcctgaagcctacatacatcttcggcggtatcccgctcacccagagaatctatgagcgcgtgtatgcagcggaacggacggcgaacgaagccccgctgcttgccatgtcgaagcgaaccagcaccatccacgttgacgtggagaaggccatcgcgaacgaagaggccttcaacgctcgcctggcgttctggatcgccaaccgtgataaccacggcgtgaaagttctgggaattgacgaaggcatggagcagttcgacacgaacctggccgacttcgacagcatcatcatgaaccaatatcagctggtcgcggccatcgccaagactccagccacgaagctcctcggcacttctccaaaaggattcaatgccactggtgagcacgaaacgatttcttatcacgaagaactggagtccattcaagagcacatattcgacccacttcttgaacgccactatttgcttctggcgaagtcggaagaaatcgatgtgcagctggaaatcgtctggaatcctgtagactccacgtccagccagcaacaagccgaattgaacaacaagaaagccgctaccgacgaaatctacatcaactctggcgttgtgtctccggatgaagttcgcgagcgtctgcgcgacgatccgcgttccggctacaaccgactcaccgacgatcaggcagaaaccgaaccgggcatgtctccggaaaacctggccgaattcgagaaggccggtgcacagtcgacaaaggcgaaaggcgaagccgagcgagccgaagcccaggcgggcgccgtagagggcgcaggcggcccggttcccgccgctccacgcgggactaagcctctcgcgaaagcggccgaggaaggggccagcgaggccgctgaaccgccgtcgcgaccggaccccaaggccgagctgcggaacttgttggtcgaccttttgtcgaagctccaagacctggacgacattaaggcgccggacggcgtagacatagagcacaatgacgcgcctggcctgaagcgcacgtcccaacctggcgtgtcgggcatggaaccttcggtgttttcgtccaaccgcatcgtcggtcctcgtgatcattcggaactccaaaggatcaaggttaatggaataaccaccttgatcgaaaatccgcgtggaagcattcggcaaggaaaggatgggagctggcgagttcagatgaaacatcactacggattcatcaagggaacgaaaggcgctgatggggatgaagtcgattgcttcgtcggtccgaatctgggctccaaacgggtcttcgtcgtcaaccaggtgaacaaggaagggcagtttgacgagcacaaatgcatgctcggcttcaacaacgtcaatgacgcgaagtctggatatctgtcctgcttccgtccgggttgggatggtctcggctccatccacgaagttgacctgcccgctttccgtcgctggctggcgaacggcgacaccaccaaaccattcgggggcgagtgatggcattcaaggcctccaaaaagcgcgagcgccgggcacctcttccagttggaagagggaagcccataattccatcggcaggaatcgaggcctggtatcgaaagcagatgaaggacatgtccaagctcatgatctccgactatcgaaacgagattgagaaggcgctgtcccagcctgcggccgaacggttcttcgccagagacgaatccgttaacgtcctgttcaagatgacccttcgaagcctacagcagcgatggagccgcatttttgaaggtttcgcggccaagatcgccccggagttcgtcaaccggaccgaagaagccgccactgctgcgacccttcacagcttgtcggtggccggcgtcgatcagccacgagctgcgtataatgagagcgtcaggaacaccctggaggccgcaaccacttacaatcatactctcatcaccaagatccaagaggaagtccacgagaagatttacacatcagtaatgctgtctctgacttccccgaatccggaagagcaaggaacttccggcataactaacgcacttcgcaaagtcgggaagttttctgaagatcgaatcgaactcatcgcaagagatcaaacaagcaagctttacagttctctgagcgatgagagaatggcggaaaatggagtcgaggagttcgagtggctacactcttctgccggcaagactcctcgccacacccacctggagaaagatgggaagaggttcaagctgaatgaccccagactttgggagggtccgaaagcagaccaggggccgccaggatgggcgattaactgtagatgcagaaagataccagtcatttgacatcgataggagcgctatatgccgttagttcatggcacttccaatgaggctcgttctgaaaacatcaagcgggaaatcgaagccggtaaagacccaaagcaggctgcggccatcgcttattccatccagcgcagtgagaaagggaagacggcgaaagattgttcgcctgagctcgtcgccgatcttcgcgccctggtggacgctctgtcgaggctcgtgaaatgaaccgcaagacgtgccggcgccgactcgtggtcgatgtaatcagggccaatattcacggcggattcttcagcctgaagtttgccgccatcgatttggcaatcatcggcgtcgccatcttgatggcttttggccgataatgctgagaaaatctggattctgactaaaaattctagtccggatagccgcaagttaccgtttacggaaaatagcagtaatttggaaggcctactgccgcgaggctttaacagagccagttcctaatttccgatttagccgcatgcttcaaaagtatatagcctgggaaattagaagtaacgttacaatagaattcatctataagtaacgttataatataacgtcaatctatatgctctagacgtattgaaattcaatttttaatcggtaaattggtaatttgaattagtttaggagttgaaagcctcgcggcagtaggcttagacaaatcccgtcaagtttccgagaccaaattaccggattttcgcggctgaggaaactggtaattagatcataatacaaattataatgtaagttaacagtcacggctacatctaattattgttccgcttatttacccttagatgtactgcgtatataatacagccatagtccacgactcttcgaattaacgatggcaaagtcgaaaagaaaaattgacgaaaatggatatatgaccatagagggctgcccgatcagctcttatggcgttttccaatattctgctggtcaactcggtcttccgggcgatccgacgcggattgtcaacgtatatcgcccggagtctgccgttagcgatccggagtacatcgaatctctgaagaatctcccgttgatcgacgagcatgagatgctgtcgggattcgacgatgatgacgatagcgtggcccccgaagacaaaggggtggagggcatcatcacatccaacgcttactacgaagccccatgggctcgcggcgatatccgcatctattcccgcaacatgcagaatcagctggaaaggggcaaagaagatttgtccctaggctatagttgccgctacactgagcaacccggcatctggaacggaacgccttatgaagtcgtccaggacaagatgcgcggcaaccacatcgccctggtaaaagagggtcgtgtgccgggggccagagtattggatggtctgtgttttgaccatctcagttttgatttcagaccatccgatgagggtaatgaaatgagtctcaagaaagccaagcggaagccccctgtccagcgcgtagggcaagctgctgactcggcggtcgaagagttgcgcgccctgtggccgaagctatctgcgtctgtccagaagttcctgggcgaagaagagcaggagccggagcatcaggaaggcgcagctccggccgaaccgaccgacagcgagcacctgaccaagcatccgactctggaaggtgctcagaaggatgacgaagagcaggaagaggagccttccgttgtcgatccggccgtggccgccgtcgagccggagcatcaagaaaacgccgcatccgaaatgtccggtgaaggcgaagtcgccgaactgatctctcaggtcaaagccattctggctcgactggagggtacggtagccgaaggggcagacgaagagcatggcgaaggtcaagatgtcgtcgagggcttggaggagcagagcagcctcagcggctcgcaaaccgccagcgacgatggtggtgagagcaaggataacagcgaggaacttcctgaaatggcacagaagaacgcgcaagatgctgcaattcgcggtctctatcgcgacattgctgctaaagatcgcctctacaagcgtcttagctccgtggttggtgcgttcgatcaccgagctatggactcggctgaagtcgctgtttacggcgtgaaaaagctgaacatcagctgtgcgaagggccaggaagctctggcgctcgacatgtacctgaaaggcgtcgaagcctcgcgcggcgcggccagccgtcaatcgaaagcccaggattcggccggttctgctccgcagtgcgccgagctggacagctacctgaagggggagtaactcatgttccagaaacaagtttaccgccagtacactcctggttttccgggcgatctgatcgaggacggcccgaagcgggcgcggccgggtcgaatcatgtctctgtctgccgtaaatccggctgccaccgccaccggccccaaccgcatcagtcgcgctttcggttacgccggtgacgtcagcgccctcggcgaaggtcagccgaagaccatcgcggctcgcgcttctgaagtcgtgatcggcggcgccaacttctttggtgtcctcggtcatccgaagcactatgcgctgttcggttcggccggagactccctggctcccagctatgatctgcccgatggcgccgaaggcgagttcttcgacatggccaccggcctggtcgtcgaaattttcaacggcgccgcaaccgccctggacctggactatggtgacctggtcgcctatgtaccaaacaacctgcctaccgccgacaacgcgcttggcctgccggccggcgccctggttggcttcaaggctggctccatgccgaccggcttggtccagattcccaacgcacgcatcgttaacgccatcagtctgccggcccagtcggcggggaatctggttgctggcgttaccatcgtccagctcacgcagtaaggaggcgtcatgagccagatcagcaagacccattcgcgcctcgcaggccgcaatgcgaaacctttcgacctgaaaaacatcaccaatgacgccgtggcgtctctgcgccgcatcggcctggtattcgatcacgccgtcgtccaggaccagatcaaggccttggcgaaggccggcgcgttccgctccggctcggccatggacagcaacttcaccgccccggtgaccacgccgtccatcccgactcccatccagttcctgcagacctggttgccgggcttcgtgaaggtcatgaccgctgcacggaagatcgacgaaatcatcggcatcgataccgttggctcctgggaagatcaagaaatcgtccagggcatcgtggagccggccggcactgcggtggaatacggcgaccacaccaacatcccgctgaccagctggaacgccaacttcgaacgtcgcaccatcgttcgtggcgagctgggtatgatggtgggcaccctggaagagggtcgtgcctcggccatccggctgaacagcgccgaaaccaaacgccaacaggcggccatcggtctggaaatcttccgcaacgccatcggcttttatggctggcagagcggcctgggcaaccgcacctatggtttcctgaacgatcccaacctgccgccgttccagaccccgccgagccagggctggtccactgccgactgggcaggcatcatcggcgatatccgcgaggccgttcgccagctgcgtattcagagtcaagatcagatcgatccgaaggcggaaaagatcaccctggccctggccaccagcaaggtggactacctgtcggtcaccacgccatacggcatttcggtttctgactggatcgaacagacctatccgaaaatgcggatcgtgtctgctccggaactgtcaggcgtccagatgaaagcccaagagccggaagatgctctggtgctcttcgtcgaagacgtgaacgcggccgtcgatggaagcaccgatggcggcagcgtgttcagccagctggtacagagcaagttcatcaccctgggtgtcgaaaagcgggcgaagtcgtatgtggaagacttctccaacggcaccgctggtgcgctgtgtaagcgtccgtgggccgtggtgcgctacctcggcatctaaccgatgcctattcaccaaaggccgggtttccggcctttgttcactctgactctgactcggttgtaggggccggttagggcataattactaggactacgccaatgactgtttacatcgtttccgccatgactcaatccgtgtcttacaatgcgtatgacacctctgatccgtccaatcctcgccttcagcggaagattctgattcgtggccgcgccggcatcgcatcagaaacttccggcttcggcgacatgatttccgacgcggccgggcgtccgatctggaccccgcagggcgattgtactgccgtgagcgattcggatttcgagctgctccaggccaacaagattttcatgcgtcacatggataagggttatctgcgagtcgtgaagaccgacatcaccagtgaccaccagcggatttccaaagagactcgcaccatggagcgcgatggattccagcctctggacgctgctcgtctgcagcagaaaatcaaggtgactaccgccagcgcttcccaggaacaagagttccggatttaaccgagggtttcggtatggtgattttcgacgaacataagtttcgcacgctgtttccggagtttgctgatccagccgcttatccggacgtgcgcctgcagatgtatttcgacatcgcgtgcgaattcatttctgatcgcgattcgccataccggattctcaacggcaaagccctggaagcgtgcctgtatcttctgaccgcccacctcctgtcgctgtccacgatgcaagttcagggcgcggctggaggtggcgtcactgccggtgggacccagggcggtttcatcaccagcgccacggtcggcgaggtcagcgtggccaagctcgcgcccccggccaagaatggttggcagtggtggctttccgggacgccttatggtcaagagttgtgggcgctcctcagcgtcaaagcggtgggcggattctacatcggcggtcttccagagcgccgaggcttccggaaggttggagggacgttctggtgatccctggtgcgaatcttctgcgaatggccttcggggtcatcggtactcaaattgtgaaatatcgcaagtttgagcagcgagtgaagaatgatcaagctcagtacgtttccatgttcgaggagcctttcgacctggcagcgtctgttcagcgagtccgacgcgatcagtatgtccagtttaatctggagttccaacggaattacgttatgatcttcgccaactttgagatggttgacttggatcgcgatgtggccggtgaccagttcctctggaccggaagagtctttcagctggagtctcaaggctcctggttttatcaggacggctggggagtttgcctggccgtggatatcggtgcggccaagctcactgatgacgggaaaccgactttctaggtgatgtatgtttgacggcgaactgatagcgaaactggttgtcgagttgaatgcggcgatgacatctgctcaagaggctttgcagttcccggattttgaagtcgtccagaaagctcagccgacccaacagggaacgtcaaccaggccgaccatctttttccagaaactgtttgacattcctcgcggctggcctgccaccgattggcacctggacaacacggcccgcaaatatgtagaaataactcgacagcatgtagagacgactttccagatcagttcccttcattggcagaatcctgaaataactcacgtggttacggcttctgatatcgccaactatgtgagggcttatttccaagctcgatccacgattgagcgcgtaaaggaactggacttcctcattcttcgcgtgtctcaaatttccaacgaagcattcgagaacgacaatcaccagttcgaattccacccaagttttgacatggttgtaacttacaaccaatatattcgcctgtacgaaaacgcagcatattcggccgatggggtattaataggcatatgagtctgaggcgcgattcagagctaatcgccgcgcacctccagatgttaagagccatgcgcggcaggtccgtttcagccggatggtattccacggctcgatatcctgacaaggcaggcggatcggtcggaatacaagtcgcgagaatcgcacgtctcaatgagtacggcggaactatcgaccatccgggcgggaccaggtatattagggacgccattgttcggggccggtttgttggcgttcggttcgtcagaaacgactttccgggagaaaccgaggtaactaagcctcacagaattaccatcccggctagaccgtttatgcgatatgcttggaacttgttttccgcagatcgcgccgcaatccagaaccggatagccatgaggctggccagaggacagatcactccagatcaagctcttgcccagatcggcctggcgttggaaggatacatagccagaagcatcaggaccgggccatgggtggctaactcagcatctacggtcaggagaaagggattcaacagaccgctggtcgatacggcgcacatgcttcaatcgattagcagcagagtaacataaccaggagatcatccagtgatcagtcagagccgttatatccggatcatttcgggcgtaggcgcaggcgctccggtcgcaggccgaaagctgattctgcgcgtcatgactaccaacaacgtcatcccgcccggaatcgtcatcgagttcgacaacgccaacgcagtcctgtcatacttcggcgcgcagtcggaagagtatcagcgggctgcggcttatttcaaattcatcagtaaaagcgtgaattcgccgtccagcatcagcttcgctcgctgggtaaacaccgccatagcgccgatggttgttggtgacaatctgccgaagaccatcgccgatttcgccggcttctcagcaggggctctgaccatcatggtcggcgcggccgaacagaacatcaccgccatcgatacgtccgccgcgacttctatggacaacgtggcgtcgatcatccagaccgaaatccgcaagaacgccgacccgcagctggcccaggctaccgttacctggaatcagaacgccaaccagttcaccttggtcggcgccaccatcggcaccggcgtcctggctgtggcgaaatctgccgatccccaggacatgtccactgccctcggctggtccacctccaacgtcgtcaacgtcgccggccaggctgccgatcttcccgacgcagccgttgccaagagcaccaatgtcagcaacaacttcggttcgttcctgttcgccggtgcgccgctcgacaatgaccagatcaaggccgtgtcggcctggaacgcggctcaaaacaaccagttcatctacacggtcgccacttccctggcgaacctcggtactcttttcaacttggtgaatggcaacgccgggaccgccctgaacgtgctgtcggcgactgctgccaacgacttcgtggagcagtgccccagcgagattctggccgccaccaactacgatgagccaggtgcttcgcaaaactacatgtactaccaattccctggccgcaacatcaccgtttccgacgataccgttgcgaacaccgtcgacaagagccggggcaactacatcggcgtcacccaggccaatggccagcaactcgcgttctaccagcgcggcattctgtgcggcggtccgaccgatgctgtggacatgaacgtctatgccaacgaaatctggctgaagtcggctatcgctcaagcgctcctggacctgttcctgaacgtcaatgcggttccggcgagcagcactggcgaggctatgaccctggcggtgctgcagccggttctggacaaggcgaccgccaacggcacgttcacctacggcaaggaaatcagcgccgtccagcagcagtacatcacccaagtcaccggtgatcgtcgcgcctggcgtcaagtccaaaccttgggttactggatcaacatcaccttctccagctataccaacagcaacacaggcttgaccgagtggaaggccaactacacgctgatctattcgaagggcgatgccatccgcttcgtcgaaggatcggatgtgatgatctaatggtttgcggcggactcgatccgccgcgaccttccataaatggagtgaggaataagcaatgatcaacatttctgcgttcggctcgatctgccagttcacggcaagcagaactttcccgaacggattcaccgtcaccgagtttgccgacgatgcagaccccatcgacagcccgccgttcactgcggccgataccggcgtcggcctcaacggtgacatggtagtctggaaccgggcgaacatcctggaagtcgtcgtcaacgtcatcccgaacaccgagggcgagcgcaacctggcagtcctgctggatgccaaccgcaccgggaaagacaagtcgggcgctcgtgatgtcgtcggtctggtcgtggcgatgccagacggcagcaaaatcacctgcaccaacggcacccccatcgacggcgttctgatcaacgctgtggcaagcgtcggccgtctgaagaccaagccgtatcggttccgattcgaaaaagtgatcaaagccggtactagctgatgaagaaaattccgctgacagcagtccccaaccaggcgatctcattcaacgccggtagcagctattggaagattcgcctgtaccagaacatggacatgatgaatgccgatatcagccgcgacggcgtgatagtttgccatggggtccgctgcttcggcggaattccgcttctccagtacagccatcagtaccgacctgactatggcaatttcgtcttcgaccgcgacgctgattggacgttgttcggcgacggaatcaatctgttctatctggacggcgccgaattcgcagagtatcaggcgctcgccacgaggaaagaatgagcacatcaacgatcagaaccgggatgaacaatgatatccttttggccgacaatggaaacatggtcatcctcagggatgtcgaagcgtgcgcccaggacgttcgggcggcgatgctcatgcgcaccggcgaaaacattttcgatgtgaactccggcgtgggatatttcgaatacatcttctcgccgcagaaaagctatgatgacgctcgcaaatccatcgcggatgcaattttgtcctcgccggatgtgaccggcatcgagcaacttgacatcgacatagccggggaagtcttcggcgtcgatgcgaaagtcatcacaatccacgggcctgttaccacaggagtttgaaatgagtaccatccgcatccaatacgccaacggcacccaactgttcctggacggcaaaaatccgccgccgctggacccgctgccctcgtttaacccgtctgtcgaagatctggaaggcctggaccgcgaaaagaacatcgacaagggcgactcctcgccggccggtattcccgttcccccggtaaacgtcgattccaatgtcgacaacggcagaaccatcccggctccggtatcgaccgacgctgctgcggccgaatcggccccggaaggcgcccaggaagctcctgcagcaggccaaggcgacgagaaaggcgccgaggaagccccgactacagccccggtagaaaaggccgaggaaacggcctcgccggccgctgaagaggaaaccccggctcccgccaaggccacctctcgcaaaaccaccagcaagtaaggactcgacatgatcaacgtcagcggcttcggcacaggaattgtaatagtttcagcctcatcgttcccgatggggttttccttgtcgaagttcgctgatgatgagagtccgatatcctccaaagagctggagccgttcggatatgagatgctttatgacggcggcctattcgcctttgacaaggctgctcctctggaagtatctgtatccgtcatcgcagggagcgaggatgatattaaccttcgcatccttctcaattccaaaaagggatcattcagatttcttccgggcatcatcccggatatgacgactctcgtggccactcttcctgatggcggccgcaccgttctgtccaatgggactatcatcaagggtccggccatcgacaccatacagaacaccggacgacgcaaaggcaacacgtacacttttgttttcggtagctatcttggcgcccagactgcgcgtcaagccatttctaacgttatccaatcggtactggaggtggtctgatgttagggattttcaccagcctcctaagttcgcgatctttttcgattgtggaccaaaacacaaaccagctagttgctgcggatttgaggataagccgggtcaacacccggttttcttctgtagggcaacgccacatgctggaagatggcaagaccaagatggattccagaacgatccaccctatggaaatcatcgtcgaagtattttgcccttcaattgatgtcgtcgatcagatcaatcaattgctcctggatcgcgacactctgtacaaagtcatcactcgcggcatggtattcgaacggatgatgtgtaccagcgaagcgctcaatcagactccggatatgatatcggcgactcctgcgcggctgacattctcccaagttctcgtccagaatcccaagcctataatgttcagaaatgcaggggactcttctatgatcgaccgagggctggccctagctgaagacgtggttggctcggccggcgatctgttcgactacgcagtaaacggcgtccagaacgccgcagacttgttctgaggtgccaattgaactctttcctcaagtctattctcaacacgcctactctcaccatacgtgatgatgttaccaaacttcctgtctggaagagtcttcaagtcaagaaagtggaaatttactcgccggcttccgtagtgtcgaaacctctggcgacgaaagaccagacggaagctcaagtgtacaccgaagctctggacattgatgtgaagaatggaaagatcatccaaccggtgcgactccgcatcaatgctatctgtccggacctgtccaccgttgaaagtatcatgaacgctttcaatgataacacctcgactttcgctatcacttctaagtcgatattggccgataaaatggccatcatgacgctcgatgtagatcagtcgccagacatgttgaacgcggctgagatcaatatggaattcgagcaggttgagcctccagtattgaatgaattcgatcctgcgttccctcaagatagtccgacttatggggtacagattcaatctctttctgatgctaatttgctggatttgggcgccatcggcgattcgatatcttcggccgcaaaatcgctatataatcgcgtgaccagctacttctgaggatgtatcatgcttgaaatcaatcttcccgatggccgtcaaactcgcgtacaaatcgaggcgtggtcggcattggatggctgggaactccagcgccgtttcgtcgaattcgctgtcagccaagattccgacttccgccgctctttcaccatggaaatcctgggctatgcgaaagtgctgcttggcgacgatgacaccggcattccgctgaccaccgcggcagtcatcaacaaccacctcggccactggaagaacgtggaactggttttcaactctgttctcaagcacaatggcatcgacccggccacgcacgccgaccggccggactattgggaacaagtcggatcgcagatggccatcgcatttctggccgaggcgtccaagctcattggtccagcaatgaagatcgccgaaggactcgccaacaagccggagtgattcatgtctagtgatttggatgaattcatacttcggtatgaggccgatactgccagagccgagcgcaatctagagcgcctccagaaccagatcaggcgcgtgaacagcgcatcgacgagtggccttcaggatttgcgccacttcgcagacggcgcggccaccgaactcggccgagtcgttccgcagattgattctgtgacgagcgcaattcgcggaatgaacgcgcaactggcaataggcgccactggcgtggccctggtcgcggccggcgtcaaggcgttcatgaacaccagggaccagtacaatcagcagcgcatccaggcgatggacatcggcatcgccccggcacgcttggaagagtaccagagaaaattcgttcgccagtccggtggaaccatcagccgcgagcagggcgcggagatgaccaaaaatctggcagacactttccggcgtgcttatcgcgatatcgggcgagtcggcccggaagcgcgaattctgcgtatggcaggcgtcgatgtcgggagcttccaaaagggcatgcggccgctcaacgacatcattactgatctggccacgaaaatggccaagttgaagccggacgaaatttctgcctacgctgatgcccttggcgtctcgcgagactacctgagcaccctggcgaagatcggcccggccatgggaaaagtcactgagatgacgactgcggaactccagtccagggtccagggcgagtccaacatccagaaattcaacgatgccctggcgaatctcaaccagacgttcactaccctggaaaaccgagtcggcgaaaagctcgcgcctgcgttcaccaagctgatcgaaatcatcgacaagatagtccaggctattcccaatgaagtggaaagcttcgcgaaggacaccaaatcccgttgggaagatggagtgctcgggaaggcgaccgttggcagtgatatcctatccctcctcagccccggcgccctgctcggtcgcctggcagcgtgggggactcgtcgcggcatggaagaatcagggctcatcgacaaggataaggttcccggcactcaaaccagcgaagacctggccaagaaacaagaagaccaggacaaggctactaagtccatgaaagagctagagaggctggctgaccagaccacgaagtctaccaatgattttgcggtggcgatcaacatgttcagtggcgcggtatcgtcgttcgcgaatgccgttgacgagcgccaagcttgggcagcctgggcgggggaaattggtcgggcggtcggtatgggaagcaccgcgccgacttcgcgggccaccggcgtctatccgcacgcgatctatgatcagtcgaagagtggcgcggccggacaggtcttcggcgagccaatcggcgcccagtctcttcgcaatcgcatgttctcgccgcagcgcaaggccgaaccggtcaccgttccatcgtacatcaacgatatcatcaaagatgcttcgaagatgtacaacattcctgagctggacatcaagaaactcatatacaccgaaagccgattcaacgccagggccaccagcgaagccggagctaaaggcctcatgcagctgatgccggaaatcgccaaggcgtatggtatcactgacgtatatgaccctcgccagaacatcctcggcggaacgcgcctactgcgggaaaacctagatcgagcaaatggcgacatgcggttggccttgacctactatcatggcggactcgatccgaagaactggggtccaaggactcgcgcatatccgggtttggtaatgagcgctccaatcgagctgatggaagaggctcagcgcaagcagaaggctgcggccatgaccgtcgccaacgagacgttcgccccggaaggcggcgacatggacattcgtccctatgatggcgggcgactggaaaccccagaccagggcaaaaaggaagatgagcgccgcgaagcccgtcgatatgacgacagggttgtgcgaccggaaattcgcatcatcgaccgcatgccagaccgcagcgacggcgaaatcctcaagatgtctagacgtcaagaggccgaccgggcagatcctggattccggaagttcccgaatcaagttcgcggcgagaccaagcggaacatccaggctcaactcactgccggagccatcgcgcaagtgatcggcgttaacccaaaccagatcatgcgccgcgaaatcagccgttctgacttgctgttcggatacaaccaagccatcctgggcaagcaacaggagatcaaggccgccgcgaccgaagccaacaacgtattcctgtctccagccaagcttgccgaagccacggccaaggtgaacgcagcatcgcgagaaatggatattctcaggacgtacggcgagcaacttctgaagagcgctccagagcgcggccaggagctgaccatcggtcgaatcgacatgttggtcaacgtcaccggcgcgaattctccggaagaggctcgcgagatattcagtaagcagactgcagaccagctgactacggcaatccaggacgctcaaaacgattccgcaactaagatactctactgatgaaaaagagaattctgcgagtaacattcaacatgccttatggacccgaagtcatccgcgaagatctggatgttcgggtccggattatgaaggctgcgttgcggattcagaaccgggcgacgatggaaattttcggcctcaccactcagcttcgcgagtctcttctgtcgcagttcaccgcgtggaagcatcggcaacgtcaagtgggcatggaagatgaattgatgatcaaggtgtcggtggaagccggctactctgaccaggggcgcgagcaagtttccagagtctttgtcggcgaagttgccattgtcgatatcatctctccgccaccggacattggaatccgcatccagtgctataccaggcaaatcgacaggacgaagaccattcggaatatgccgcccgccaacacgacgtttgtcaagttcgtcgagtggggcgcgaacgaaatgggattgaacttcatctgcgataccagttacaacgatcaagttctgaagaatccgggccggtccatcactgtcgcgtcggcaattctggcgtcgattcaagatatgtacatgccggatgtggccgcgttcgtcgatgatgacatattgatcgtgaaggatcgcgataaagtcattcgtccggatgaggtgaccaacgtcaactcgtttgttggaattccatcttggtctgaatggggcgtggaattccagtgtctgtttgagccatcgattcgtgtggctggaggtgtagcggtcgaatctctcatgaatccaagcgtcaatggtaactatgtaataactgctctggaatacgatttggccagtcgggatcggccgttctatatcaaagtcatggggagtccagcggcataatggccagggaaaataaatcgttcaacatgttcggagttcactatacttcgcggcaattctctgctgtcgatgggctcagcatgatgtcggaaattcagagcgtgccgccagaagaattgctcaagtgcaccgatgtattggcgcatccggaagaccatccggaaggcatctggcttccattgactgctgcgaacatcaatctttatgtcgttgatcgagcgaaagtaatagctcccgtacaagttcttgcacttctgtccgaagtggtaatcgactggaactttggttttctcaaagactggaccggagtcaagattccatccagatttgtcgaagatatcaaaagcgtgaagacggcacattcaccttctgtagttgcgagcttggtcgcgaacggctcagcctctatgcgcgagttggaagagtattattcgactcaagatgcgtttaagatgatagacatcatgacggcgaagagcgtgaacgaggccttggcgtccgaagcatcgcacaacagaatcaaaaagggataattcctaaccgggcctgggaaggctatactagacctgccaaatcagaggctttcccatgtccaatatttctctaacatccgcaaaagctcccgacaggacgcgactgatcgccgctcttgacgctcggtcgcggcgggatgctctagatttcgaagtaatgataccggctcaggttgttcaatatgacagggccgagaatatcgcgacgattcaacctctcatcacctgggttgacacggaacacaacgccgtccaaaggcatcagctggttgatattcctgtaatttccatgggggctggcggcttccacataagtttcccgattcagcaaggtgacataggctggatttatgcggccgaccgcgacacgtctcagttcctggagtcgttgtcaatgtcgaagccgaacaccggccgcatccataagttcgagcatggcctgttcatcccggacgtattccgtcgatacaccatcaattccgaagattcggccgctatggtcatacaatcgacttccggagcgaccagaatttcgattcgcggcgacaacatcaagatcactgcgccgtcgaatgtcaccgtggatactccgcaggcgaatttcactggaagtgtgactatcgccaacaccctggttgtaaacggcgtcaacgtgaacaaccacggccacctcgaaaacaacccgcctgatgcccggacgaaaggcggcatgattgcttaaggagattttcatggcaagttttgatttttctgatttaacagcgggggggggttgtaatggctaactacaactacatcgtcgatactggtgtgatagtcgcagataccgccgacgttctgagcgacgttgaggccgagttccgcgccgccctcggtgccaatatcaacttggccgcgagcactccgcagggatcgcttgtcgcggccgaggccatcgcccgttccagtgtcatgcggaatgaagcgcgaattgccaataccataaacccgaacgtttcattcggaacgttcctggacgcgatctgtgccttgatggggatcgagcgcggttctgatctgtcaaccttcggctatggagttcaagtcaccggccgcagccaaacccgcatttccaccgggtctagggtcaagactccggccggcgcgatcttcacagtgatgagcgatgtcacgatccctgctggtggtgtcgcgaccatcgatatcaaatcgcaggagtatggcaacattcctctgccggtcgggaatctaatcatcatcgacggaacaattggatggtccggagcaaaagtcatcgcctccactcgcgtcgatccgggcagccgccaaatgagcgatgcagagttgaagaatgcccgcgttaatcgattggccatccaaggtcgcaactcgactatggccatcaaggcgtatgtgagcgccgttccaaacgtcacgtcggtgaacgtaatcgagaacaacaccggcgccgttcaagtggtgaatggagtctcgtttacccttccgtatgctgtttgggtctgcgtcgccggaaacccggataaacaagcagtcgccgatgcgttgtgggccgcccataacggcggaactccatgggactatggtgcgacgaacaacggcgtcccggtggacggtccgaatggcgttcctgtgcgcgatccggcgtccggtcgaaagtatgtcgtgaagtggaccactccaattatgtacgatggatatgttaacgtaacagtccagcaaggttcctcctctgtagctccggaagccattcagaacgcagtggtcaattacgcccaggggaaagtggaaggggaagaggggctggtggtgggcgcgagcctgtcggcattcgaagtcgccggcgccatcgctcgcgaaattcccggcatctacatcaaactctgccaggtcgcgtgcgtcgcggctggctcgccggctccggctccgggcgatttcacttcggaatacgtcatgagcgcattcggccaggctaccatttccgttggtaacgttcgggtgactttcgtatgactctgcctgcgtacaattcggacatccaacaagctctgaaatggctccataaccaggcgcctggaatcaccggcctgatccagagaaaggcgcaatggtatgacagattcagccgccaattctgggccaactgggagcgcgacgttttccacttgaaaactgccaacccgttcggccttatggtttggtgcatcatcctcggcacgccttcgaaagggttcggcctttatccaaaaaacagttcttgggcattcggtcggctacgccagaacttcatctatagcggtacacaagttccgccaccggcagacgcatcgcctggcggcaacttctacggtggcggcaatgccgaaattctcaacttggacgaaatcaggaaagtgcttcagctaagatatgtagcgctgatttctaacggctcgattgcatatatcaatcgaatgttgaagtacatcttcaatgatgacgagccttgggacgaggcgaccggtctgtacttctatctaatggactcaaccggcgagaatggccctgtggagaacttggcgatatatcggaaagattgggaaggtatggtgctgttgtccagttcgcccagaacgaaccatgtgctgacatcgacccctgccagcgacgccgattggccgggagtcgatccggccgcgagcggtattccggtaacggtcgaaacggcgtccgctacggccccggacggctccgctacggtgtgcaagcttactaagccggccgggagtactgcttacgtctccgcgccgatagatgggccgctggggtccggtagcactgtaacgttctcgttcttcgcgaaagccggctccacccgtttcattgcaattcagtcggctgccgatttccccagtcgagccgatgccgttttcgacctggattccgggaacgtaatcagcgatcagatgctggacagcagcgtggtaagcgcccgaatgattcgtctggaaaatggctggtggcgttgcgttctcacgaccaagaccgtcagctcttcgttccgcgctgcttacgtcgctccggcagaaaccaacttcagctggattgattcgaattccagcgcggagattgacgtgcttatctggggcgctcagatcgaactgggtgatactccaactggatacttggaaactaccggggcgcccgtaaccatgaccgattatgttttgcagaacgcccagaccggaacagtcaagttcacacagccccttccgaccggagtagaagcgtattggactggagactggaaaggcgggtctgcgaccgagccggccagattcgcagtaggggatgggactcaagatacattcaatctgtccagccctgcatacatcggcctacccactagtggggcgttcaagctagaatacagagttggtccggcgcttaatttgtcgccgcaattgatcaacctcatgaatgaccgggcggtcggtatcatgccgacttgcgccggttgcgatgtaaaagtcattcaggagtaatgacgtgatcacacccgaactgatacccagtccgtttgctgcgcagggcgacaaagacccaatcccgcagacctcttccactggttttgccaaccttcgcgacggctacacgccggactatgaaatcagtctggcgtcgaacaacccgcaggccaaagcggtcgagcggaaaattcaaaaccaactcttcttcatcgcgacccagaacgcacaggcgtggcagcgacaaatggcgccgccgtggtttcagggcatgcctggcggctacgaacagaatgcagaagtcgtgcgcgtcggaaatgacggcataatgcggcgttatcgttccatggtgaatgccaatgcgagcgaccctctcagcagcacgacttgggaagaacaacccgcatggtcggcgatgcgctccaacatcccgatgccggccggaggcccaggcctatcttctggcggagaagtcatcacgaccggccgcaacttcaacgacctgttaaatgggacgtgggagttcttctctgattcagtggttatcgcttctcagaatgcccccgtatatccggcttccgctggtgccgctgctggcatgttggaggcgaaatcttgggtgtccggagccaatacgttctgcgtccaacgctacactgatcgcgtcgggaacgtcgctgtgcgcgggcttaatgccggagcgtggaccaactggatgtatgcggtaaacgtcatggcccttcaacaaggtcgggtcacctatggagtcgcggccggatcggcgaacgcttacacgttgacgctagttccgcagctccaaggcggcctggtggatggcatgattcttcgggtcaagttcaacaccatgaacaccggcgcctccaccatcaacgtctccggactcggcgccaaagccatcgtcggcgcggccaacttccctctcaccggcggcgaacttggccaaggactcatcgctgagcttgtattcgacgcagcaggcgaccgctggagaatcctcgcaggcgcgccacgcatccaagttggcaacgccgatcaagattaccaggcccccagctggaaacaggtgaaggattacgtcgcgtcccaaaagctgactgaagtggactgggctgacgtcgtcaacaagccgaacgtcgctatccaagacactacgccgtggttcgccaatctggagttgtctgacgctcggcctttcatcgatttccacttcaacaacaaccgcgccaaagacttcgactatcgctttatctctgaagctgatgggtcgatggcgttctactctcgccaggggtccgctggtcctacccaggatatcctgttcagcaggtcgaatgttacattcctccagccgcgactggatgttgcgaaaaacctcgcgtacatcgcgaactctggtcccctttggcagaacaccactgccgatcagcctggttggaaattcaccttcgcacaaggtgtggacgccaacaacaacgcggttatcgcagtcaataccaccaaccctgacggctcttatcgctcgcaggtcatgcgatgggactgggcgtccacgaacgtcatattcaacaatcgccctctgtttgctggacaatatgtcccttgggactccggaaactttgatccggccaccaagctcactgtcgggactaccaacaacatttcgggaccgaccggaattcgcaataccaccagcaataccggaaatatgaacacatggggctccagctccacaactgcatcgtatggaaacgcagcccttcaaatcttcggtagagggggtggcgagcctgcgaccatctacttcgacaactcccaaaccggctggtatttgggcatggacaaggacggccagctgaagcgagcaggctggtcgctcggcaataactcctatgtggtcaccgacgagtcgaatattcgtttccacgtgaattccatggctggcactccggtatggggcggaaacgaattctgggggccgtggaacttcaacccgaacaccaagttgaccatcaaagccggcacgcaggaaactagcagtactgctatattcagcggaaccctaccgttcgccccaatcgcgtctctgtccgactattcccaggcgccattgacggtttacaactcgccaaccggtccgtctgcaaaaccggccgtcatcgcgttcattcgtcctggcaactggggcgcgttcttcggcatcgataccgacaacaagctgaaatggggcggcggatcgctcggcaacagttccagggaaatcgccgattccagcaacatcatgaatctttgggcggccaacccgaccgcgccgtcctggaacggccaaaccgtttggcgatccggaaactttgatccggcgacgaaagtggatttgaacgccgcgaacgccaccaacggcaacatgatcttcaaccgcatttctggaactggtagcggcatagcttcgtcaggtcgagttggcgccatcaacctgcagaatggggcgcattcggggcaagcggcagcagtcactttcgagcggggcggcagtatcttcgtcaacttcggcttggataccgacaacgttctcaaagtgggtggcggaaacctgggggcaaatgcctatccggtcatccacgccgggaactacaataactacatcaaccaggcgctggttcaggtgggtcttggcggagtcggttcctatggcatctttgcggttctggaccgtgccgctcgaaccgcgaccgttcaaccgggagtggttgtggacggttccattctcatctactcgtcttgcgccgcaaactacaatagcggtcaaaggcctgccggaacttggcgctgcatgggatatgtattcaaccgggatgcccacaatcctgactccgcgacccttttccagcgagtgacgtaaaatgaaatggacgcggatcagaaaccctcgttggctggatgaaacaaacatccacgccatggtgactttcgatgggatcggggaagttcctttcaccgccaatccgcacgacgtggaggcccacggaagggccatccacgctgcgatcctatccggggcgcacggacctatcgccccggtcgatgcgaagcgggagcaggccttgcaggacgctatacgggccagggaaaagcgggctatccttcgtgatacccgctggcccatagatcgtcacgacgagcagaggcggcttggtatcgaaaccacggacggacctgggttgatagcagcccttgttcactggaggcagcagattcgcgattggaatagcggggatcggccgcgacttcccatggctctgaaaacgatgttcaaaaatcaggagtattgatgaaaatcacaaaggatattcttatcaccggaaccgggtgcaccacggatcgggcgatcaagtggctggatgatgtccaggcggccatggacaagttccacatcgagtcgccgcgagccattgcggcctacctagccaacatcggcgtcgagtccggtggactggtgagtctggtggagaatctcaactacagcgcccaaggattggccaacacatggccgcgccggtacgcagtagacccgcgagtccgcccatatgtcccgaatgctctggctaaccgcctggcccgtaacccggttgccatcgccaacaacgtgtacgcggatcgtatgggcaatggttgtgagcaagatggtgacggttggaagtatcgcgggcgcggcctgatccagctgaccgggaagtcgaactattcactgttcgccgaagactccggaatggacgttctggagaagccggagcttctggaaactccggccggcgcgtcgatgtcttcggcctggttcttctggcgcaatcgctgcatccccatggcggaatccaacaacttctccatggtcgtgaaaaccatcaacggcgccgcgccgaacgatgcgaaccatggccagctccggatcaaccgttatatgaagaccatcgccgcgatcaatcaaggctcctgatattcgcccaaaagaaaaggccgcttattcagcggcctttttgctttccggctttgcctcttcaaccatcttgacttcaaccggcgcggcggactcttcctgagtgaccgaatccacatagttccctagtgaactcagaacgccgattaacagcgctcttaccaccttgtccttgactgtctcgcctatgatcttggtcagaacggatattaactcttcccggagtctggggctgattcttggccgaaagcgcttgcgatgctctttgcgtttcatgtttagtcctctgtctgcggtcttctcctcaccccgataatggcttggggatgcgctgtgttaatcggaaggttcgggcgctattataactcgtcgaaaatgctcgcgcttaactgtttaacgatacgcaccgcgatattaaatcgccttctttctggccaaggaactctggcggccgggtccggtctaaggcctaatttgtcgacattaaaacgagaaaacccggatcgcctgtagggcaatgcgtccgggtttatttctatctagtgtacgctaggatcagtgacttccgccccacccgtccagccagcaatcgaagacggcgtgcctcggcttgtccttggcgccatgggagaagtgcttgaaccggatgacctggcccttgagatactccctgtcattccagcggcgctgtttctcgtcgtgggtcaggctggaggccgacacgttgaaggtgactccaggccacagacgttcgttgcggcagacgaacgcaccgaccattccggacggcgacaggttttctgcgtggctggagcgggccgtgtggccaagttcattgatgaaagcttcattgttgttgtgcatcaactcttccacgtcgatgatctcagcttcatcgtagtcgtatcgcttgactttcacacagtgcccttccttggcagtcgagcgaccgaacttgtacagtccatcagcgcgcttgcccatggaaccttcgaatcccagcattgtgtggcggcgctcgacttcgctgaactgttcgatggaagtcaccagttcctgctcgaccaggtgaatcctctcatagccgaggcagtgacgaaggaagttcacgcgctcggccgccctggctaggcgatcttcggtcggtgcgcgaggatcggtgaaatcatcgaacacatggaaagaccagtccggctcaccgctgtggcggcgaagatcgccggacgacttctggaaaactttcgggtcgctgatgtcgccgcagaccagctcgccgtccaggccgttgaacagtttatcgctgaggtattcgtagatcgattgattgttctgccgcttgagttgacgagtcagtgcctcgccttcgaatacgaagcagcgaaatccatcgatcttcggcgagaaatacatcggcagttggccttccagcagcttcgggtcaaaattcgatgcgagcatgggtttcatacagttctccagaaaagaagcccggcgaaccgggctaaatggcggtaagccggctcagatggtttcgttggcgtggttcagttcggccatgatcgacgcatagcgctcgtccgactccttgatgaacacgccgtcgtacatgacgcctttgcgatccttgatggtgtcgtaggccgcagcgtagcaggcgagcatggtagtgtcgtgctcttccgtcacatcgaacagagtcatgacggccaagaccaggctcttgatggctaatccatgatttccgcgtgccagcgcgccggccagatgtccgaggtgctgcaaatcgtcgccgtaggtgggatgaacttcgacagtctcgctgaagaccgatagatgatcgaacagattttcaccgagttgcgcggccatgatcgtggcaaccaccattacgtcgccgatgccgtctttcacctcgactggtttgttataaacccaggcttcgcaaacttctgcgaactcttcgaccaacttcaggaattgatccttggccgaagagcctttgatcaggttccggtcggaaccccatttaacaaccaggtcgtgaagttcgcgactcatgatttgttcgatattcattctttcgattccttttggatttgggattttactgcgttgattatggacgccgtgctctggcgcgatccatccttggtggtgccgaagtagaaggccattaccgacttcagctcggcgaaccaatagccgatgatggtgccgatggcgaccgaggatgtcgggtccatcagagcctcgcgcccgaatgtgaagattgcgatgatgatgagaatggagcctgtcagaagagcgaatgtaatcgccgggcgaacgaaatcgttctgctgcgccgcaagcctcctcgcagagtccctgtcggccgcctctgcggcgaactggctaagctcggcctggagctggttctgctcagactgaaggcgattttgttcggcttgaatcgcaagctcctggagacgaacgcgctcggcgctctggagttcggcgagacgcgctagagcttccggattcgcgtctagagcgctagcgaccgaggctgggtcggccttcgaccccagcgccgtcgcgacgatagcgccaacggcggcgcctgcaggcccacccaggagcgaccccagagccggggcagccgcgccgatcttactacctatgtctttccagtccatttcgactcctcaaaagaaaggcgccattacagcgcctttctccggccggtgattcagaactcttcggcttcggtcgcatcgccgacgccaccggcgtcactgcgaggctgttcctgcttgctgtagtccaccttcacctcgccgccgacgaacgacttgtacaggtcggccgcagccttgaagtgatccgggttcttcaccaggccttccagctcgaactggacgccggaccagctacccttgtcgttcgacatgcctacggtggtcatgcggaccaggttggcgaaagtcggcggggtgcgcaggccctgcggggtctgaacctttttctgggacagcgcggtcatgagcttcttcgaggccttgatctgcgaagacgacagggagatcagagcctggccgaaatcgccggtgtccggatcgatgacgatgacgtaatgaccacgggtgtcggcgaagtagtcagacttcttgtcgctgaccgaaccgtcttcgttcggcgcgtacaggcgtccttcgacttccttcaccttggtcgggtctttcatcatttccttgaagtcttcgacgctgatggcgcccttgaaaccgccctcggcctcgcgaccggcccagcggatgaactcgcggcggtacgcggccggaatgatcagcaggccggtcttgccgtcgtaaatcttgccggtgacggtgttaaggaacatgccggccttcgcgccttcgatgtacttcgggtcatcttcatcgacctggggagacatcttctgcagaacctggatgaaggggatggcataggactcggcatcggcgccttcgaaaccggcgccgtcatacgcgcccaggtccatgaagtcgggaacttcggtagtcgcgacggcgccgccaccggtggcaactgctacggccttggtttcttctgttgcttcggaagtttcggttttcttaccagccatgttaggctccttgtttgtcgaatttcagttatcgctaactgtgggtttataataacggaagttgcaactaagtaaagcaaattacatattaagatttgctctttttcaccttcggtttcgtgattttggcctctttgtattcgtgcacgccgatgaaatctggcagctcttcgcccttctccaaatactcgcgaccgaacgcctggagggtctggtaatggacatcgcggttgatggtggcgtcatagccggcctcaatgatagcctcggccgccttcttcgcatcttccatttcgccgcgaccgaattcggccagaaccttggtcttgatgatgccgtcgttgtcggtttcttccagccacttccagaacttcgatttgttctcttctttgacggaaatgattgccttcggctcgactttgaccgtgcgaccgtccgccagagtagtggtcttttgaccaagctcctccaacagctccggaatggtgttacgcttgagggtcttcagctcctcttctttttcggccagcgccttctgaagatcgagaatttcgccgtccagctgcgaagccttgtccaccaagttcaacagtcgatggccgatgtcggtggcttcaaccgccatttcatccatgacgccaaaatagtcgatttcgcccggcgcgttctctttcaagtattccgggatttcaagctcttgctctttcatgtccgcctccaacttagtgatgttcccttacttgaactaagtattgagtagatattatgccgcatcttccttgatacggctactgatttacatattaaatttcgtcgcgagtgctaacgtcagcctcaaacacgccatcaacgacataacttgccaggttgcgcttccactccagactgacctggattttctcgtcgatggaatccaaacagatcaggtcgaagtacaaaaccgagttgacggtgccgattcggtgatttcggtcttcggactgcatccgcaactcattgtcttcgtcggtcgtgtagtagatggccacgtctgcggctgtgagcgtgatcccgatccctgctgcggccgggttgcccaggaacacctggacgcgcttggcctggaaatcgtcgatcaatttttcccgctcggcctctttggtctcgccgtaatatgtgccaaacgaaatcccttgcttctccagatattccttgatctggtcgatttcctggattcgcatggcccagatgatgatggaccgttccgggtcttcctccagtagaccctccagaagatcggtaaacacggcgaatcgcggattgtcttcgggcggcaggatcaccggctcaccatagacattgatatagcctgacgccacttgcttgagcttcgagcgcgctgccgcagcatcgaaagatacatcaagcatgaaatcttcgttcttgagaacaaagtggtagtcttcttcaacgcgctggtaaacctttcgttgctccggcgacatttcgaagtatatgcgcttgtacaccttgtccggcaggaatgggagcgcttccttcttcgtgacccggaaactgtgcggctcgatcagggaccgaagcttgtccaggttccggaataccgggcgcccacactcgtctttttcgaccagttgcggcggcatggtctgcttcccttccagcttgcgcatgatagctatcattcgaggatcgtcactgggcaccaagacggaaaattcggccacaaacgcccgataggatttcgtccccagaattccattgcgcaagaattggaattgcatgaacaaatcggtcggtgcccgcgtcagaggagttccggaaaggatgcgacgggccacggccttctcgcccagctttacgatctttttcgcacgcttggcttgtgggtttttgatcctcgttgactcgtccacaattgcgcaaactttgaatgtatcaaggaatcgctcgacctcatcatagccagcctgatggttgattgcgtcaacgtttatggcgaagacgcgaagaactttttcatctgcgaacgtctcggcatacagacgatccaagcgcgctctggcctttttggaagtcggtcggccgcgccaatccacgctcaaagtcttgatcgcgacgtgggtgggaatctcgcgcagaatccagttggtgtggacgcctttgggggcgacgatgagcagcgcgtccacgcgcccctgtaggaagaggcgaacggcatcggccaaagtcgtccaagtcttcccggtgccctgctccatcaggtatgcgaaattccgtttgttcagggacgcggccagggcattgaactggtgctgcatggcctcggtcttcatgcccttgacgggatatgttttggctttcatttgttctccaaatcggcgaggaactgaacaatgttatccagtccttctgcatagctcgcaacctccaccaagtcgcggctgttaagctcgaacaaatcgagcatgggattcagcagcagccaatcggtcccgatcttggccaggacgaagccccggccgccccagccgatccgttcccgaaggaaagggatttgcccaggctcgaaacatctggccattgggcaggtggaggcgcgtttcggccactcctccaacgccttgaactcgacccagaactggacacctcgacgattcaggcaaatcgaatcggacataccagaccgccgcgtctccagaaagtcgatcaggattcggccgagtgagcgttgcttaaacgcattcgccgctttcgtttcgcgatcattcatcgccttcgccctctttggaaactttctctgcttgcgctgccagctttgctttttcgcgttcggtcaaaatcctcttgacggccttcacgatgaacatatcgattccgctgagcttccagcctttgatgaggaaccaagagcctgtcggcgttccttcagcaatctgctttccgtattgcagatatttttcaggcctaattctgaaacgtatcggctgatcaaccgagtcgtcaacacacatgatgtcgagaaactgcgactggcccttgtacaccgggtttttcccttgatctgccctcttcttctggcgaatcggttcgttctcatcagacagaacttttttcaccatcttgacaatgaccaggccatcgtctccatcgcggatatcccgaatgttctggatggggtttccggaagttactccaaccagctccggattgtcataagcatgaccccagagcgtgtgagcttcgttcaaatctgcgaattgaacttcagaattcgacagactcgcggcgactttctcccaatcctgaagcgtcttcaggtgggttccggccagctctttatattgagctttcagctccctaagatcggcctttaactccttcagatcggccttcaacagtttttccagttctttgtcttggctgattttcgccgaaagaatctgggcttccagcgctgctacatcagcagccttgtcctcaacatcctgtgccgatatcgggcaattggccagggcgatcctcgcggccttgacgtcatcacgaaggcgcaggaacttctctgccttcgccgggccgaagcctttggcgttcatgatgccgccgatcaggcgtccgtccgccactacccagttaagttcggaatgctccgggtccagggccgtatattctacgccttccttggccaattcgcgaaggattgacacagtttgctggtcgtctttcgccgcccgaagacacgcggccgcgtattccaggcgatgataccgcttcatgtagcaggtccagtacgtcaccacggcgtagctgaccgagtgggagcggttgaatccccaggcgccgaatgtcaccatttcctgccatacacggtgagcatcgtccggggcgacgcctatggtcttggcgccctcgatgaacaattcccggcgcttgttgaagaactcttcgccctttcgcgcagacatggccttccggattgccgacgtttgttcccagtcgaactggccgatgtccttgacgatggacatgatctgttcctgatataggaagacgccgtacgtccctgacagatattgctcgacctgcggaatggtataggtcacaggctcgcggccggccacgcgctcgatatacttggtggccataccagaagacaacggacccggacgggcgagcgccgtgatgtggtcgatgttttcgaacgcggtgatattgatcgcgtttgcgaccgaacgaaccgcctgcccttcgaactggaaaatgccggacattttgtcttcgttgagaacatccaggaccgccttgtcgttcaaaggcaagtcgtacaactcctgcgcagtcacacagttcgcgtcctggatgacgcccagagttcgaaggccgagcgcatcgatcttgaggagattcaaatattccgaatccggcttatcgagctgcgcgacgccttcagaagttaccgtacagaagtcgataacttcatcgttgcagaccaggatgcctgccgcgtggactccagagtgggatgggtgaatttcgaggtcgcccatgcaggcggacgcgatctcatacttttcacggaagtcgcggccgggttgagttttttcgaaagtgtcctccaatccttttccataccgttcgtccgccgacgtatattcaatgatcgagtttttgatgttgtcggtatcgtggaaggggatgccgaagcgctttccgacgtgagcgataaccgacgcggccttgagcgtattgatgttgcccaacttcaccacgttccaagtgccatacttctgctggagatattcgaacactagatagcgatgggtatcggcgaagtcgatatctatatcgggaagatcggaccgggaaatgtcgataaagcgctggaagagaaggcgatgagggagcgggtcaacctcggtaatgccaaggagatagcagaccaaagagccggccgaagagccgcgagctgggccgaccagcatgtgcttcttggcaaacgcgaccagatctgccacgaccaggaagtagctgtcgaaatctttcagctgaatctgcttgatctcttcttggaatcgatcctcatactcctgagtccattccttgatgtggccgcgactgaggcggtaggcttgcccctcgcgagccagagcgacgatatcaccatccaggtggatcataggcgccttcgccagtttcacatccgccaattgttcgactaccgcacgagtattggcggcggcggcatcgaactcttcgcgagtcattatgtggcgtaaacggctccacagctcctcttcggtggcgatgtggcgaaggccgaccgattcccgcaccttccaggccgacgcgaaatcagcatggtcgatggacggcatgtcgttgtaagaggtaattaccaccggctttccgaatgccctggccgtctccatagcgccgtgcgcggcgaccatcgacgcgggattgatgtcaatgtagtcgattccggccagatccagatgggcataggcctcgccagcgaatttgatgacgccgtcagcttcctggaattcttgcggagtcaatccttgattctggaccgacttggaagtcaagcggtagaacttcttggtgtccttggcgagcacccaggctttgagcttcagctctttctcgccatcgtcggcgcatttgatcgggatttccatgccgaatccgcgaggcagttctgccttggtagcggcttgttcccaacggacgtggccccatgttccatcatcgacgatggcgacgaatggggattcgatctctttggcgcgctcgatgatctccgggaatcgaccatatgcggcgccgtaggagtaaccagagcgaacgcggagctgagggaaagacatcatgcggcctccattgcttgatacgcccgatacactcccatgcgcttgcagacttcgtggagcagccgcacgtcgtcgagcgcccggtgtttctgtacatatgggccgcagtagtgctcataaagatgttgcagccgcatgcggtggccgaacaacggcgccgactcttccacagtacagatatcgagcgatgggaaattgacttcgtccaggccgaacttgccgcgagccagatcgcaggccagcatgaacttatcgaatggcaggttgtgggcaatattcgcgtcggctctggaaaagaagtcgcgaactttctggcgctgatcgaggaaagatgggtgcttgatcaggtcttcgttcttcaacccggtgatcttggtgatgatttcctcgataacgattccaggattgcagatgaactctacttcatccaaaatcttttcgccatcagttatcacgccggcgaattcgattattctcggctgctttctcagactcaccctttggtggaacgggagtcctgtggtctcagtatcccatacagcgaatatcatgtttgttccctcttatgtcgaaaggccggctgctttcgcgaccggcctgaggagtataccgcgacggctgaagatttacgccttctgtccgtctttcggcgtgatgcggccggagcgcatggtggcgtgaacaaacgccgaatagttgatcgagtccaaggccgaatcggcatccttgaacccgctattcgccaggcgagtgagtttacccaccatgtgcatcacgaacagggcgagtcgatgatcatcggcggtcttcgccaccaggccgttcgggaagaggatttccatgatctttccgtacatcagatcattgcgaccataggtgctctggcgggcgcggaagacttctgctgcggcgtacagattgttgagaacatcttccgcgaaatcatctggatgggaatcatcttcgcccggccagacggattccatggcgaacggcgcggcgtcttcggtcgggtcttcggactgagccgtttcagcgagcggagacggggctttcgcaaacgcctcgtcgagggtaggggcggagttcggggcggccgggaacggctcgcctgccatgtcgttgggcgcgctatcggggtcgctatcggccgcgacggcgaagaacggcgcatcgcagccttccaggttcaggatgtaggaatcgacccccaggctgttgtaggcatcgatgatgtcctggcgatcatcgaatgccgcgacgatcttggtgacgccttcgattttcttcaggatatcgagcacaactgcccgcttgaactgaggcgccggctcggtgttaccatacggccgcatgatgagttcgtactcgcgatgttcggcgatgccgagttcgcggtggatcttggccctggtctggaaaaagtggttgtcggttcggccggtgatgaagaagatcatgaggtctgcgtcgatggcatttctgatgcgacctacagcgtgcgggttgagagcgtccttgtcgagacgagaatgatactcgtcccattgccgttccaaggcgaagctcttgcggtggctatcgtcgaagacgcagccgtccaggtcgaagatgatgatgccattcttgggtttgcgttccatattcagatttccttggtggttgctttctgagtgacggttttctcgatgaacgtcccatcagaagtgaggcggaaaacttcgccgttctggatgaaatcgaacgattgtacgttcatcgtgatgcgaatggattcgctgccgttggtaacttcgacttgtgcgatgtcgccaactttctcgacaatgatcttcatgttacatggacttcccgttgacggccacagggttggcctcctgtttttccgatccccagaaatcgcggcgcagctcttcctgctcggccgagcgatccatccaggggcgatagaacttgcactggtagataggttccaacggaacatcaaccacaggaatctggccgggcttcgtctccagagcggacatgaaccggtcataggatttctgctggatttcctcttcattcatcaccttcgacccatagcgcgggaaggcgcaggagccggtggcgacgcagtgcggctgaagcaggctgtcgaacatcggatagacttccagaaccagccggcgcatttcgcggaacgcttcctgatattcaccctgggtccgaacgcacaggcgaaccttcgccatgtcgctgagagtccgcagattgaacttggccgcgatcttcgtttccatgttggaaggaatgattgcgcgagcgtcctggagcgacgcgccggcctccagcagcttatggtaacttgtctgcgcgtctgcgatggcgtcatgccacaggcggttcagctcttcgcggacatggtaggtcgggtcaggctcgccatttaccgtagccggttcgtcgaattcccagcggaaagcttctggttgaacgacggcgctgatttccagagcgcgactggtttcctgctggtaagccccggtacgagtacgaacgagttgatgggtgaaattcttgctgacaccctcgatctggaagatgaagtccacgaactcgaacggcgagcggatggtgtccagcatgtacttccagtggtcgagcttttcggcctcggtcatggtcgccgggtcttggccgcgcatgcgggtggacttcgttcccaagagcaattcccaggcgttctgggtgtaactgatcagagaaatcttcatcagaaatcttccggaattggcgtgaagtggaatttttcggtgagagccagggccaggccttgggcttgctctttacgaaggccatactgctctatctcttccttgagcagttcgcaggcctccagacgcgactcatactcttcagcctcctgaacggatgagaagacggttccatcagaggttcggtacacaagttcaatcgacatgatgacctcagtagcagcggatgatttcggcgcggatatctcgacggtcgaggtagtgctcacgaatcttatcgcgggcgcgctcggcctcttcgcggctgccgaatgacaggttgaacgacgtgaaaggcttatcgtccagttcagcgccggtctgatacaggatgcctaccagaacgaaagacggcgcggtcttcggctgctggtcggcttgcatttcgaggaaggacataggaacctcttcagaatgatctggtgcgtaaattaatagcgctcctgctgagcagctacggtttccggctcgtagatgagcatgtcaacgatttccgggcactggcctttcacccagtcgatggccgcgaccagtgtggtggaagcggagaaagagaaagtgcggtaagactcatggatgtacggcgaccccatggaatcgcgctcggtcgtgcgcgaaatgactgctttgatattaacgatccggcccatcttcggcctccactttagcgattatatcggacaggcttaacttctcgccattcaggaaataactggcgcgaaacttcctgtcgccctctggcccgacgattcgggccgttattgtgagactcccgccgccaagggcatcggtgtcccggaagaaagctagcagcgcccgcttgagtgctgcttcgcgaggatcgacggccattaacctaccacgttccagccgtgctgagcgcaccacacggcaccggtccctgcaggaagactattcagaaggaacacaggagtcaggcggccatcctcggtcatatggacgaagtagcgggcgccttcaccgagccattcggccttggcgatggcgcgttcgagattggccttggtggcgtaggtcttggtgtggttcttgtcggtggagaaggttacttcgcgggccatttgtcgattccttttggttgaagggtttcgcgtttcgatgagggaatattacgcccacctgatccagaagtaaagcacttttgtaaattacttcacgaacattttcttggccttctgataagacgaagaagtcatcaggcgctcgatgacgtccatgtcagaaaccagatcgtccagaaggacgttgcgccaggtagcgaaccggccgagcgagaagatgccggcttcatgggtgagattccagatcatggattcgcgctcgtcgcggccgagcggaacgattttgcctttggtctggatggtcggctcgccgtcctcgatgagatgccttttccttatgccgaaggccgcgcaaacgtaatccatatcccagtcgctgttccattcgatggtttcgattttaccatctaaagtctccactatccccttagtgatggattcgacgatcagagtatcaccggtgatggacgctcgaaacgttcccactttaggaccagggaaatacacggtctggaagacatcacaggggatggaaagcttgtatcgactcacgatgatggaggttccttcaccgaatgacgggtcgattcccaggtccagccctgccgcagccagattggcgcggaacggcgcggtgctgatgatattcacgtggtcatcttgccggcgaagatactggaagaaagaggcgtcgaaaggacggctccaagtgatacgattcgcaagcttagccaccagctgctcatagtagtcggccggtgcgatccaacgcttttcagtcgccagattccagatggaccggtccgacaggccgcccgttactttcctggagtacatgttgcagtggtcgatgcgcggctgggaaatgaactcgccgtcgatgtagatagctttgtgtacagtgacttcgcggaacgggatgccggtgagttggccaatcactggcgagcggaatcgcagaagtgcgttgtggcgctccttattctccggcgtcgccgcgtcgatgatttgggcttgaggaaagcgatgcgcggcgatcagtccggcgagtccggcacctacgatgattactttctgatcaggaatcatgatgtgtcccttatgagtgtacaaaacttgagaggataaaaaagggacccattttcatgagtcccttgaagagctagacgattcaggctcagaggagcggcggcttactcctcctcaccttcggaaccatcggcgccctgatcttcaccgtcgtgctcctggccttcgtcatcggccttctcgtcatcgccctggccagcttcgtcttccttcgaagcgatggcaaccagatcgacccagcccatgatttccagcttgctcaggtagctgcggaccgaagtgccatagagcagatgggccaccttctcgccgaaggattcgatttcgaccggctcaccaacggtgcagtgctcgttgatgtatgcgaacaccttgccgcgagtcgagaaggcctgcggagtgccatggccgtcgccggtcgggatgaagtgggtggcgcgcggacggcgggagccattggacttcaggtcttcgcggcgggcttcggccttcttgcggcgctcttcctgctcttccttgcggcgctgcttctcggcttcgcgctcagccttcttctgctcggccaggcgcttgcgctcttcttcgcgagcgactttctgggcttcctgggcggccttcttctcttcggccttcttggcgcgctcggcttccttctcggccttcttctgctcgcgctcggcttccttcgccttcgccttctcggcctgctcggcttccttcgccttggccttttcagcgcgctcggcttccttggcggcggccttttccttcgccttctcggcgcgctcggcttctttcttctcgcgctcggctttgcgcttctcttcgcgctcggctttcttctgctcggcttccttggccttggcttcggccttctcggcgcgctcgcgctctttacgttggcgctcagcggccttctcggccttgcgcagggcagcggcttgttccttggtcagctcttcgccttgggtctgttcgttctggtccttctgttccatgttcttactccgggaatgtttaaagggatggcttattggcctgtgaggggattatctctaaactaattgaagaagggaataccttagcctgaactttcctaaatattttctttcgggaaagtccagactccagggaacttatttatgttcgcgaagtttaactcttgcgcaaaaacaacaagtattcaactgcacgagttatgccagtatacatcaactgactataaggaattgacggtaagttttcttccagcattgcgactcgcttccattccgatccctgcgacttgtggaaagtcatcgcccaaccaaagtcgaatccgccaatggccttctgcgcctccagccgcacatcttcctcggccgaaaagctcaaaggattgaacttcacccagcgctcatagttcgtgccgataatgcgaactttggcaaacagcatttcatccggctcgtcatcatcttcttggccttcggggaccggcttgaagtccagcagaattgcttgctcgccgttcatgatgccatattcgtgctggttcccggtgcacaccagcttctcgccgatttccggttgtacacccttgtagccgaggatgcggcgcgccctggcgttcaagcggcggcgagtattgttgtaggcgcaaaggatcacgccatcgtcatccaggaacgtccgcatttcgtcgtccgacatatcgaagccagcccggactaggatatcatcatactcgcggcagggcaggcgtttgccctggcggacgaacatcgacgctcgaacgatattcccggcgttacgctcgatttcagtcatgactgtgtcgcagctatgttcgtggaaaacctgaacgccgcgcacagggggaacttggccaaagtcgccgatctccagaaccggaattcgatgcgccaacaagcgctcttcatcccactcaccggtcattgacgcctcgtcgagcacgaccaacttcggcttctcgtcgagcgagtctttattggcgaacatgatttcgccgtcttcatcctcgccaatcggccgatagatgaagctgtggagggtccgggcattgatacatcccttctcgcgaagccttgcggccgctttcccggtaggtgcgacgaagaccgtccagtccatcgagcagcaaagttcggcgatgatcttcgcgatggaagtcttaccagtaccagcgaagccggcgagtcgatagacctggcggcggtgcgctcgatcacaccaaccgcgataccagtccacaacggaattgatcgcgtcgatctgctggctgtttggccgaaaaccgaatcgctcttcgatctgatcgacggtgaagttagatgctgacatatttgcgttctccaacgctaggtttaattgaatcgagactcagtttaagcacgccgtccacagaccatccagtatcacgacgatacttgcggccttgcggatcgacatagaagttcttcgtgcgccgcagcagaacatagtgccaagcagctccaagtgcgtggacccttcctttatacggaaaagccttcgcctgctctgcggcctccttggctccagggagccagcgtacggtcgaaaggaccaggaccgcacccttaatagcctgggaagcggcgcggccgtcctgtgggctatagcgatgtctatcgaagtctacccagtggtggttgccgcgccggagcttgaccgttcgggatcgcccctcgaacaccaccgtgccttcgtgagtaaaaatatgttccgccatcgaatgttcctttataacgtacagttatgctttacctctgcgcaggaagagtatactatcagctgactcgccaaagcgagcgaatttaatccaactttacttcggcaggaaagtggccgatactagcgccgccgcctgtactgccctccaaaacagaggatacattaaatgcaagaatgcaagatttcccgcgaccaactcccggtcggtaatccgaatcccaatgtcgacaagacccgcgacccgaacctgaagcccggtttcctgcgccgcagccgcgagctggacccggcgctggcagttcgcattcgtcgcgagctgatccacgccgaagcgtccgacttggcaaaggccggatgggtcaattcacagtccagcctctatggatcgaaagccttcccgcgccattccgtcgttcgcgttaccggagttccggaagatggggctttcatcggcatgctgatcggcttcatcgagcatcgcgagcatggcgaatgggcggtcatggaagccggaacgaaagaaggcggcgccgtcatcattccagtcgatcacatcatgcgagcgtcattcgccgaagccgaagagttcgccgaaaagtgggagcggaacttggggtggcgtctcctgcgccagctccgcgagtgcggcgccctggccgggactgaagacgagttcctgcggcggatcatcaatcgatacgttcgtgatcgcacgatcctcgatcaccacaaagtcggcgcggacaaagactacactgatgcagtgctcaaaagcatcggcgaaacatggccgaaaattccttcggggaaattcgtcggacaccgagtcgcgcagctcctgatcggccacaagctaggtcgagcggggaccattctgaatgacctggtggacttcctggagaagttcgcggccgggcgcgataaagttctcaacatcgccatctgtaattgaggtgaagggatgaacaataaaccgacgctgaatcaccaccatcaaactgcattgttgtatctgtataacaatccaaatcagccggccttcacaggtcccaacaatcaggcgctcaatgaacttcggcagatggggtatgtaaaggcgaaagaattcgaaaactgggcaggcaccggccatcttaggatggaatggacgctcaccaaagccgggattgagcgcgtcgaagtcgggcttctggggaagtgcgacgcttgcaagggaatcggtcagactttgcttcgggggaagtgtactgtttgcaatggccgcggccaggggtggatcagagaatggtcgcagaagccaatcgaagataatcctcaaattgtccctaagttcgaaaagaccgatgcgaatagacttgcagacgccattgaagaaatcggccggctggaaaaggcactggccgaatccgaaaagcgcgggagcgagctggccgcgagctattgtgacggcgtagtcggcgatgaatacggccatccttattgtcgttataaagtggaacgcgacgccgccttggccgaagtagagcgcctgcgagaatcaaaaggcgatccctctggcagcttcgacaggtgtatgaagatgatgtacgagcgcgacgagaatgcaaaacgactcgacgccgccttggccagggtcgcggagctggaagggaagttgacggactgggtgcacgaagggttccggctcaacgaggcgctagcggtcgccaaggctcaacccagcgtgccggaggggtggaagctcgtaccgatggacccgacatcgcagatgacctctgtcggtcagtccctgcgttatgacgcggtaaacagcatcggcgagatttaccggcaaatgctcgccgtagctccctcacccatcgatccggccgcgcatcctcagccgtgccagcaaccacaggcccatcctgcccgctgcgggtgcgagcggtaagtgccaagtaaggagtttatgtaatggaacagcagaaaccttcggaagtagatggagccatcgccatgaccgatctcgatattcgcattcttcgaaaggcgaagccagaagcgcaggacgagtgcgccgtgtccatgttcgcaacggcgatccgccagaaactgcagcgctcccgcgataaaggccgaggcggctggatcgactgcgacgaagatattctgatcaacggattcgccgaacatgcgctgaagggaaatgagaacaacctcctggacctggcgacgttcctgatgttcatgtgggtgcgcggcatcgatgatgcgaagattcccccggcgctcgaaaaggcgagacagcacaagatcatggaagcctggagtcgaatccatgaagacggcctgaactccgccagaaaggcgagcgctgcgcgacagttcgtggaagtgccgcgacgcaaggggcgcccggagcgacttgcatgaagactcacgaaataagactggcccaggccgaagagttcctgcgcgaactcggccgagggattccggacgacgaacgggtaatggtcggctacgctgaagaggccacagtccaaaccgacgagaacggccgcaagctcaacgccggctggtggcctgtgccctggaaggaagggaaatacatcaactccagatccaacgcctacgcatgcatctcgtcgtccatcaagacgcccaacccgaagaccggccagatgcgatactggcgcggcgaagcctctttcggccatggcctggcgttaatggtcgatgacatcggctccggcaaagggtccaaaggcgacttcgaccgcgacgagttccgcgagcgactggagccgaccgcgattgtagagacttcgccgaacaactaccagttctggtatttcttcaaagagccgatgtcccacatgctccagttcaaggcgctgctctattcgtttgtggaccaggtgctaaagaaaggcggcgacaacacagtcaaggacgtcagccgttacggccggatgccctttggcttcaacaacaagcgcgggaaagacggcaagttcaaatatgccgacgaaaacggcaagcccgaactcgtgcgcctgtacagcgccgactattccaagcgctactcgccggaagagatcgcccaggcctttggcgtccgcatcatcatgccacagatgaagaaggtggagataaaccgcgacgattgggtgtatgaccaagtatggctgaagtatgccgagcacatctgcacgaaatacaagatgggcgaagcggcaggcggccaggtccagcagaatatgtccggcaaataccgcatccgctgtccctggggcgacgagcacaccaacggcgatcctttcggcgcatactttcgcggaccgatccctggcgccgagcacgaatatgttttcggctgcggacacgatacttgccgcaaagagcatcgacggacgtgggcggccttcactgatgaagtcgtcctgccgtatattgtcgaacaacttgaacgcatcaaccgccgtcacatcggcgaggagtagtaactatgcaaaatgatcctggaattctgattaccgccatcggctcattgcttctcggccttatcatcttcttcgaaggcctaaagggatggaaaatacaagtcgcaaacttcctcgcgtcgcttctgtgcttcttcttcggcctttctgctttgacgttctggttcgtcgttgcgtttgacgtattttaatcgacgaacggtacagaaattttcggatggggacggaacttattagctatgccggtttaggtaggagataatagccgtccctttcgcctcaatgtgtagaggcaatgttgaatccgatcatgtaaagcagaaggcggcaaacctaacatgattatcgacgaagataatatttttgatgatggcgaatcagggtccagtgagtttgatctcacgcagatagaagatgctggaatggaccctttgatgaccgccgcgagcaaggcggccgatgatgcgattgcgaggaacgaaacgcatcgcgcgcaaaaggcggcaagatacgccgaggcgtatgctgaaccagacttgaggaagcgagcgcggttgttgatgctcgaccaggcgttcgatcttccggtcagccgggtggtgaaaggaccgttcgatgacttcatcacgaaatacagctcgacgtcagacagcaactacctcgcggtctatgatacgttgttctgtaagggcgacggaaccgtcccacacccgcacttcgacgagtttcgcggccggctggtggaccatcgcggcgtggcgttcaacaacaagaccctcgatccgattgacctgatgggcgccctcgcggctgcggccttggacgatccatcgatcaagaagacgattgagacttgctgcgtgtgggcgcgtcgatatcgccgcaactctttgatcgagacgtttgagcagaagataccggagtgggatggcgaagagcggatcgacacgctgctgatcgacttgttcaaaccgttcgacaccgaacttaaccggatggtgagcaagtatttctggctgagcttgtactgccgcatcaactaccctggaatctcggcgccgatctcgttggcgttgattggtgggcaggatgcggggaaatcatatttcggcttgctgatctgtaaggaactgtccggtgggcgcgatctggctccagttcagctcgacctgagccgacacgaccagacgccgttccttcgcaacatcaccggcaactcggttattgcgaacgtcggcgaaatgtccggcttcaaaaagggcgacatggaacgcatcaaagaattcctggtgcgatcttcggatacattcgatcagaagtttgagcctggcgaaacgatcaagcgacaatggatcaccatcatggacggcaacggatacgatggactccagcgggacgactccggcaaccgtcgattctatccgatgttcgtggcacaacttcccgacgaagatggaaagccgaactgggttaagccgggcgatggtaatgaaccgttcaaggtggacttcaccgacttcggccgcaaattctggcaagcgatggctgaatgccgcgcatggatcgaagagcatggtgtcgatggatacttgaatatggtgtctgaagcgaaccgcgaagtgcagaacttctctatttcggaaatggagaatgcgcgtggcgtagttcgtgacgatacgattgacatgtatctgatcaatgttctgatcagttgcgagttcgaagagatcaagcctggcaggaatgccaagaatcctggatggagggctgacaccgtcgccattctgaaatggttcgatattctcgccaggaagaagccgatttctcgtcaccttactccgcacctgaaggcgctgggattcatcccgaataagaacggcctggctggttggtgcttacctgtcgataaggttgcgcctggatggacgaatggcctgcacacgacgttgccgccgttcaatgatgcgcttgtgtacttgctgagaaagggcgacccggatatcactgatgaggctgccatggcaaaaattcgggcagtccgggccgagcgagccaagattttgggcgaggatttctgatgtagtggtggagtgtacttggactaggccgccttcgggcggtcttttctttggtgcggcgtacattcgattccggtggtggccgagtgagattcgaaagctatccggtaattagggtgggttggcgacggtgtattgtggaaagttcgtggacgttggatcgaggattttgtgtggtgagtggatgaattgtggaaaatcgacgattctgactggatttttggggtcggtcgcgtcgagcttaccggtaatttacgaaaccggtaattagggcctagccttagtgccgcgtgggtttgaagggctttaacggatactttccggtttcggatgcagccgcaaaagtatactacagcgaaaat

SEQ ID NO: 3 - DNA, 45770 bp, synthetic construct
  misc_feature 1..45770: note = Nucleic Acid Sequence of ECACC 17062003 Pa222
  source 1..45770: mol_type = other DNA
ccccagagtcccccaggataacgttggggtggcctatccccaggggtatagcccctagcccctccctaggataccataaggctagtccgggggtcaaccccttgagataccgttcatcgggtggaccctagggtattcataagaaaaatcttagggtattcagggggtggatcggggggaatatcccttgatcccctattgacatcccacgataccctatgttaatcaaagactgtctatatccgggggtttgctgggggactgatagtccttagactgtatgaaaaaccagtagacaaccccgtaaagaatatggcgtaatggcgccgccttgagggaaaccgataggcaccgggggaaaccccaggggcttgacaaaccccgcggaaccgggtctaatggccccacgttctacctaggccaccatcccccttgccgcagtgctatccgggggctgccaaccagggaagagcgcccaagtcgcccaagtgtaaagccggcaggtatcgggggttgacaagggatcggtgaagcggtatagttcgccccacgtcaacggcataacgtgcatgactccctcgggattagcgtagccttgacaagcaagactgatggccactcagcaaaagagcctatgccagagaactggacgaactaacccccgtcagagggttgacaaggcaagcccaagcctctaacatgggcacccatcaggtgacggccacctggggaaagtgtaccttcgacggtccttggactgaacaagggagaaacccgaaagctgagggaataacgcacactccgaaagagcgtaggtcctggccttgtaccatagaaggtccgactggtagcctgtcccagcgcaatcaattgacagaccccgtggttcaagcgcgacggggagtatgatgggttagtgtcgaaggcttagcgcagagggcgaagcttggtagatacggcgaaggtaggggcgagtggatacgaagacccccgaggataaccgaggacagaccataacgacgacactaacgcggaaaggggccggctacgccaatgccaaggtttgccgataaacccctttgtcgtatcgggatgtgtgtcccggctgatgattcctaaaggatgaaacgaccatgactcaggctcttgacaaaaagctgcgtcgcaaggccaaccgcaaggccaaggctttgggttacaaccttgggaatctggggaaggcccaacagcgtagtgagcagaagtttgggattattgcaagttgtaacaagatcctcgacgacaagaccacttcgttgcaagaaaaggcaggggcgcgtaaacgcaaggctcttatgagtaccgactggcgtaaccgtgaggttactaaccttcggaactggtataagccgagcaagtgcggtaactctgccgtcatcactgtcgaagtaaacaactgaggtaattacaatgagcgactacaagcgcatcaacgggatcatcaagaccattgccaaccggggtgctgccctggacaagctggttcaaaccactggcatggatatcctcaagcacatcgacgagcatggcgaggtgtccttggcctgcaagctgttcaacgcgatgcctcaaggctcccgccggctggccctggcccactggttcatcgacaacggcaagatcgaggccaataccgacaaggaaaaggccaaggaattcccgttcgtcttcgccaaggacaaggccactcgtctggagcgtgccgcagagaaaccctggttcaagtacaagaaagagcgtgacgtggccgacgagttctccctcgatcaagccatcgccgccttcaaggccaagatccagcgtgccatcgacaagggccagctccaggcagcggacgagcgtatcgccgtgatccagcgactggaagtcaaggacgaagcgaaggcagcgtaacgccgggtgcgccaggggtgtgtactatgacgcgcccctggtctatccctagagtgcatggtaggtccgtgcattgtagcgatagaccatcaggaggtttccgtgggttactgtatctttgggcaggttcattccatccaaggccaggattatgtttctggtttctgcgatgcaagggtaggtttcgacgctggtattgtgtaccgttggacattcaaggacggtaaatttgcctgcggtgtctactccctggatggtatccgtctgaacatctgcgggtggatcaaatgaagtcaccctacgaagcggcccatgaacgtgcccaaatgattaaccgtctcaagaaactcactaggatgatccgggtgcatcccgatccccggtggattgttgagcgtcaggaactcataaggaaactaagcaagtgacaatcgctatcattgtatccagcattggtatcgcctacttcttctttcgtgattggaaagaggaaatgggtatctaaccccaactgatgaggccaaggtgattcctggccgaaacccccaccggacctatggtcgcaggctggggcgtcttgggaaatcaactaaggaaaccatcccgtgaagcgcaacgactaccgaaaggtgaatcggaacatgcaagccatcgaggccatcgaccgcaagattgccaaggctgtccgtgagttgactaactccggaggaaaccacgttggtaaaaccctggagctgaatcggctgcgggctaagcgtgcatcgctggccaaggtaagggcacggtgagtacatggaagtctattgtatgtgccgccatacgctcacgttccaatgggttgttgctggtaacgtgggtggttctcttcgtcctgtcctcgatcttggacagcatcccttttgcatcgtgaggtagtcccatggatatcaaggtgtggccccgcaacggcgtaaccctcagttctctggttaagtcccaaacctttctcatcaatggggatgtgtacatggtctgcgaactccgaagcatcaagatgaagagtgaatctgacgaggttcccgttctcaatctcaagactggcaacgtcatttacatgcctccgttgagcctggtatacccagtaactgccgagctgaattgctacgaggtgtaacatgaaactccaatacaacatgattaccagtaagatcaaggttgtgatgtggaatgaccttgagccgggttgcctgtacatcttcgcagaggatgacgggaagaagaaccccaaagtcttccagtacctggagcttaatgatgaattcttcatctgcgaaatcttcggcggacctgagggcgatgtattcttcagcagcgacgttgactgcgggttcttccccgtgactgtcaagtacgcagaggtagaagtccatcggttcggcccaccggactagggtgtgtactatgacgcccgcccaatacatctacctctggttggtatccaaggttgtcatccgtcgataccagcctaaccccaaactgtaccccaactggtccgttactcatatccgtgtgtcgatcttcggcaaacgtgcaggtatcgtctatgaaatccactgaaccccaagtctattggctggctagcgccgatggtgaattccacctcaaggtcatcaacttcggccagttcatcaccgaaaccctttacgaactcggcattccggtggaccacaaggttcatcgggtccactgaggtgcaaccatgtcaaacaccaccttcactgtagcagcaacccgcccccggaacgaccttcacatcagtaagcgggcggatggggtttatgtgaaccctaaaaactctggcgatattgtcttcctcaaggaaggtaataaggttgtcgtcctcagccagagcgggttcagcatcctcgaagcccagaccatgcccaacatcggggaactctacccggctaaggaggtccatgtatccgccagggtatgggggccgtatgaagaataaacccatgatcggccagataatggcccaggagcgccgcatgaagcgtcgtgtagaaaagcgggggtttaacatgagcctccaggaaggcacccaggagccccgtggtgagcttggtttcaccttggctgccgtaggtgtggagtccagccgatcggcttacttgcggcacgccagggaggctatgatccaatctggtgagccttgtccccactgtatgccagtcttcggccccaaagagggtcgttgttgcaactgcgcgagggattggtgatggatctcggtaatctttggtgcctcttgggtgtccatcggtacaaaatacttgatggtggaccctacgagattcgtcacaaggggcggttagtggaactctgccactattacgacctgaggtgtgatcgctgcggtgacgtacatcggaaggtggtccgcagcaaatgatgacatacctcctgataatcccagcgatcctggcctacatgtcggtagcccttttgatatccggggtttccgcactctttttagccgaccagaatggaatggtaggccagagagaccgggacattgcaatcgtgatgggtgccctgtggcctgcatcgcttccctggatgtgcttcatggctgttatctacaacccactggtcatcctcatcaagtcaaccaaacgactaatcaaaggagattactaatgcaccgcagagactttccttcctgctgtaccgcaaaaatctacatcggcatgggaccctcgggtaccgctgaccattacgccggcctcgcatccaacgggttcagcccccgtggtttcgccaaggaactgatcggcgctatccgtcgtgaatccaacgagggccacggtacgatggttttcaccgtgaacagcgagcaggtggtagcagataccatcctccgccgcatgggcagccactacaacccctgggcatccagcgacaaccactcgaccaaggtccgggtccacgtcatcaacgtgaagtctgcggcagatatcctcatcaaccatggggttctccgtcgtcacctcggtggcttgcaggactaccccggtaccgtcgagcactcggagtacctcgacaagctctgtaaaggtctgtaacatctaggccttgacattctgctggaagtgtggtataataaccttaaggtgccgggggttgctttatatccctaaggtacctaaggttctaccttctatcttcttcatcttaagaaagaggtaagaacaatggtcctcaagctctacactcgggtaatgctcctggctatccccgcatggataagcatcctatgggtagtaccccagatgctgtccccggctaacaccctgatgaacgccatgggtgccggccttctggtcctttccatcctgtactccctctgggctgtcagccatctgtactcctacgccggtaagctcactggcaagaagggggtgtagtatggccacggtaacaagaagtagtaaggggccaacgatccctctttacttcgaacgtaagtgtgattcccaggtctaccttcgggcggactgcttccaaaaggatctcactattccgatggtaaacatctgcaatgggagtgtcctatacacacatcctcgggagattcacatattcttcgctgagatacccgagggtgaaacgctcaaggtgaaggtgtgatgtactatgacggaacagatgcgaggccgcctccgtgaggcaatccgaaaggacttagagggcatcgggggagatacctccgacctctacgacaacatacataagatagacggcctgctgggccacccaggtaagcccccgaatgattggggcttctatgtactgatgttcgtcatgttcatcgcattgtgcatggtctacatcaccccccttctctacaagtaaggaatccctatgaagaaaatcctcggcctcctggccattgctgccctcgcgatcaccgcaggttgctccaaggttcccgctggctataccggtgtcgtcgtcaacatgatgggcgactccaagggcgttgacctgaaggaaaccccggtcggctggaagttcctgacccccaacgaggagttgttcaagttccccaccttcaaccagaacttcgatctggcggtggttactgcccaggacaaggacggcctgaagctggacttccccatcggcgtcacccttcgggctgctccgggtagcgcacccctgctgttccagacctaccgcaagggcatgaacgagatcgtaagcgtgaacgtgccgcaggtggtccgtaacgccgtgaacaacgccagcagcaagaagtccgccgaggccatctatggtccgggcaaggaagccttcgtgaaggaagtcgaagctgccgtccgtgagcacttctccacccgtggcatcatcgtcgaaagcctgtacctcaacggtatgatcggtctgccggcgcaggtggtcgaggctatcaatgccaagatcaaggccactcagacggcgatgcagcgcgagaacgaacttcgtcagacccaggccgaggcagctaaggctatcgcggcagccgagggtgagaagcaagcggctatcctggtggcacaaggtaaggccgaaagcctacgcatccagggtgaagccctccgccagaaccccggcgtggtagagctgaatgccatcgagaagtgggacggcaagctgcccacctacgtcaccggcggccaagccatgcccttcatcaacgttcgctaaggaggtggacaatgggtaagcgtaaggttttctacgagcgtctctgctccaacggtgaattgaccctgaacggcaacctcaaggccaccaaggtgtcggcaccaaccaaaggcaagcaacagcgccgtggcaccttctaagtaacttagaggtcgctagtaatacccctagtattcctgtcctgggggtattgctggctaactcaaccaaggagaaaacaaatggcccgtatcaagtatgccttcggtatgaaacccaagaagggcgagggtaaagccctcaaggtaatgacctcttcctcctgcttcggtacaatggaaggtccggtaacccacggctataagctcgacggttggaccttcatctgctcccgccgaagcaagaagttcatcgatgtgctcaacaagtgtacccaaggtgaactcaagaccatcaccatcggcggcaaggccttcaaaatcccgaaggtccacttctggtcttacgaaagcaaggccaaggattcccccttcgagaagtattctaacgggacggaaatgatttcccccacccatcacggaaaagaaggggtctgcggtatctacttcaaccccaaggtacacaccctggattcctggtaccccatcatgaaattcctcttcaagatgatctccagcggactcgactaccaaggtcgtgaagaggaggttcacctgaagcttgctgagaagcatggcttctggaagtcctacctcgcaatgtccttccacgggctgatagccaatggctacaccgggtatcccctaaccaactacatgttcagtagcgattgggatgatctaaagaagggagatatccacatccaatccgctgaagagtctattcgatccggtcgttggacgcccaatcggaatgccccaggtggacgtgagtgggtaaggggtacaccctggcgttctgaattcctgaagattcagttggacaaggtggaggtagttaccatagcacctcagccaaccatcttcggaaagaaagatccctatatgaccgtagatagcgaggttggggggtttatccggcttaatcccaatagctgcgaaaaacacggagtggtaaagctggggatcatgggtgagattggctggagtcgtgacataaaggatatctcactggccctcgaagacttcatcgagaaaaacttctgacaaccaaggagaaatcaaatgggaatgtatgcagcccataatgtgtactatgacgccgagggctctgttgtacaccaatactcggtcaagagtatggccgaatatacaaactgcttcggggcctattgcgaccggacctccgacgttgggggcgaccggtgggttaccggggttggccacctgttggtatctacccacgagaatacctctggtaaactggtggagttcctgaacagtgatttggtcaacaggatcaccgacggtggtatcctctcggcttccatcgactggcctacccgttggtgggattacaacgatgacctcaagaacctcagtgatcccgaccacttctctcccccaggacgccggcaggcagtgtacgttcgggttgatctcaggaagaacgcatcggccatcatctgtgccctccgaatgggcgatcgtctgtggtgcgtaggtgattccatgcgtcgcatcacccaggaaagccgtgataagatcctgggatgcaatgcagagatcctcaccctcgcggcttgtgcccaggtaggcagcactgctcattgcgacagctatccggcgatgttcccggtgactgctggtgaataccgggaaggatgcgagagtcacggatgggaagtggatgacagctacatcagcgaagtcatggatggtatcgtaggtgggtacaaggagatcacctacttcgatatcaagagcatccatgggcggactcgcgaagagttcaaggacaagctcaagcatcacgacgctgagttgtggcaagggtataccaaggacgattacctgatcgaatgcgacgggctcgaaggtgtacacaatgatcggatcctctccatcatggcacccatcactatcgacgtaggtgacccaggtcgggaaaggtctgagaccgtacccaccagtccctacagcatccccgaaatcctcgaaaaactggagaacatgcaatgaacaacgcaatccccctgatcggtgcagatcccgaagttttcgtcggctacgaccgtaacccccagagcgtcatcggcttcatcggtggaaccaaggaagcgcccttggctgtagccggtggtgccgtccaagaagacaacgttcttctggagtacaacatcgacccggccagcaccaaggaagagttcgtcaagcggatcatttccgttcgcctcctgggtgcccagatgctccaccccttcggcatgaacatcatcgagaacctgtcctctcacctgtacgacgaggaactcctccgcagcttcggtccccaagcttacgtcttcgggtgtgagccggactacaactgctggacccgtcgccagaacgtgatgccgaacgatgcacctccaaccctgcgcactgctggcggccacgtccatattggcttcagccacatcgagcgagtcaccaaggcgaacacccgcgaggtcatgcagatgtgtgactacctcctgggcctggcgtctgtcatcctcgacggtgacacccagcgtaagaagctgtacggcaaggctggcgcaatgcgctataagccctacggcggcgagtaccgtagcctgtcgaacttctggatcttctctgtcgatctgaccgagtgggtctatgaaatggcagtacgggcttacacctccaagcacctcctggaggagtacaagtccatcgtatctggcgatgaagtccagcgaatcatcaacgagaacgacggcgccgcggcggtagctgcacttaaagcgctgggggtggtatatgaatgacctgaataatcgccatcggttgtccggagacttcaacatgtactactcctccacctatgccttcttccgggtggacggcgagcctcgggtagtgtacgtggacgataccgagtccattggtgacgaccgtcaattcgacgggtttcgtctccttggcaacgtgtatcgccccgacggcagtaactactacggaggggttgtctacagcgaggtagaaagcgtgcggccctcaagtgggtactatgacgtctttggtcgtggggttcgtgatacctatgtatccttcctcgttaacaaccgtacccagcgcaagggtgtagatccccggaacatcttgctgaatcatggccaacaggctatcaccggcgaaatgatgatccgaatcttcacccaggccgaggaaatgatctctgctccctcccaccgggacttcttcatcaaggatggggttgtacactggaagggagtgaaggttggccagatggtggatgggcggctgtccgcagatgaacaattcaagaaccaggaggacttactatgtcagttattggcacacagatagggttccgtaagaaccagatcatcgccccggaacaccacgaggaacttcctgcggttgcttccttcgggttcgaagtcgaactggaaggcctcaacaactggccagaagtggatgggtgggatctgaagggtgacggctctctgcgggagggtatggagtatgtcttctccggtcccgcctctggccagagggcaatcactcgggttgaatcctttgtaagtgcgatggaagaaacccctccggctcctaccttccgttgctccacccacctgcacatggatatgcgggacgtagagtggccggtgtacgaacgaacggtcctgacttacatggcattcgaggatgttttcttcgatcactgccagccgtatcgtcgggatagtaacttctgcatcccgttcttcagcaacgactggctggcccagaccttcggtcgccgtatcctggccccggaaggtgaccgagagaaagtcttgggtcttacctcctggccaaagtattcggccttgaacctccaggtaacccacaacttcggatccatcgagttccgtggtgctcacgccctgactactcgccaggaaatggtaggcctgatgcagcgtatgttgtgtctcaaggccttcgccatggctcacgcagaaacccccctggaagagttcctcaaggtgctctccgaggtgaatctccgtgatgtgttcttcctgggggtatctccggactatgaaatgtctccgggtggtcgtgaaatggggatcgccagtgctactctcgcggtggcaaccatgggctttgttcgctccggggtagatcctctggaggatgaacagaaccgtcagcgacgtctccgggagcaggaacgtgagcgtcagagggttttggatcgtaggctgctgctggctcgatctactacctccagactactcgatggtgcagcagagcggtacaacttggcaatggtcccaggtacccaggttcgactggatacggcgattactgcggtaacttctctgcgtcgtattggtcaccaagtgcgtgtacgagaccttctggaggaccaagaggctcttcaagatgccttcgtactgctcatggataacccgcagcaccttcagcgccataccggcgtaacaatcgaaccagatatgtactaaggagaaatacaatgtgtggattggtaggcttctgtgccacaactaacgcgagtgataacgaaatcgctcttctcaaatccctcctggccgtggatattatccgtggtgctcacgccaccggtttggccaagatcgacccggttaagaacgaggtaggaattcacaagcgggcagtagatgcctacgacttcctggctgatcctgaaaccaaggagttcctggacaagggtcgggctcgcatctacatgggtcacaaccgttacgccacgatgggcgacaagacggaccatggtaatgcccaccccttccaggtagaccacatcaccatggtacataacggaaccgtagatacctggggcctgcacctgctggacggcaatgataagtacaacgtggattccaacatgctgtgcgctaccattgccaaccacggtgccaagaagacgttcgaggagaagttctctggtgctgctgcggttgtctggtgggactccaaggaacgtagtctgaacttcatccgtaacgaagatcgtcctctgttcatggcggttaccactaccgggaccatcgtatgggcatcggagcctgggatgctcaaggttttcctggagcgacccaatgctaagatccgcctgcgttctcctatcgctgaactgaaggccgaagtcctggtaactatcccgttcacggaggccggagtgcgaaagggtgcagaaccccagaccactccggtcacgtttctggacctcccgattcccgaaagcgaaaggcaagcagcggcatggtggagtcgttacgtcggtgtctcggactacgatgactacagccgaagccaaggcagccaagcgggaacgaaaggcagccaagcgggctcgtcgtatggaacgtctggcgatgcgtacgcaaggaacaccctccggatcaacaacaacctcgacgcagcaggtagcaccttcaagcaccggcagcttgtcaccttcgatgttgtcaagatcgaggcctacgcaaacggcagcgaatacggaactgtcactggaatcgagcgtgaagaaaaccttctcatcgaagctcatggcatcaacgtcgccaaggtccacggatacaccgtcctccgagggagtatctccaatgcctacttcatcggccaagaccgtgatctcaaggttactgtcgaggatctggcggtaagctgcctggacccaaagcatcggccgactcctggggagactaccccagtgttgaggattggaacgatctcatcggagacgaaatcccattctaaacccagggttcaagtcggcggcacctcggggaacacccctccggccaacatcacctaccccctgaaggtgcaaggccacaccttcaacaacgtgcatgtctttcgggacttcgtgtcccagggatgtgcatcttgcgggaagatcccaaccgcatatgaccagcgtaatcgtcatctgacggtatacgaaggtgccaagttcactggtagcctggatgagtgcgagttcatctgtggtgagtgtgtaatcgaaaataaataggaggtcaaaatgacccaagtaacgatgaagcgtcaagtagtgatccagatggagacctacgcaacccgtaagtatcccttctcccgtgacaccctggacaagatccagtcgattcgtcgagtcaaggagcaggaactcaatgatgccaacccggacgaggaattcctggtaccggccccggtagtcattgcggaagctatcgaccgactcttcgaagactacttcgagtaaggtagtgcgttagtaatagcccctggccgacccatgccggttcctaagatgcgtacatgggatctaacttaggattccaggggctattgctggcttcactaccctcaacagaaacaggagatttgccatgttctatatctataaaggtgcccgcccctctgctggtgctgtcgctcttcgtaacgccctgggtgctcgaatccttcgttccgaggggtctacctatcggggtcgtgcgggtactgcggtaatcaactggggtaccgttggtgcagaggctcaccgactccgggatattgcccctgtgttcctcaacgacccggcagttgttcaccgctgctctaacaagctggagttcttccgccacttcgaggccaatgctccccacctgattcctcgttggaccgagcgctgggaagatgccttgggtgtactcaatatctctggtcgcatgtatgcacgtaccgatctgaacggccatagcggtcgtggtatccatctggtgtgtactgtcaacgatgcagaagttgctgcaatcgatgctctccgtagacagggccactacccggtacacatctacggtcacacccacatcccggatgttatcaccagggcacaactgttcacccagggaatcgtcggtaagcgtaccgagtttcgagcccatgtgatccgtggggaagtagtacttctccaagtcaaactccgacgtgttgccaatgaaatggtgaccaacgaaggacaaagcatcgttcgtaacgtagctggcggctgggtctacggggttaacgatgcaatggaccgtgatggtgctgagcaggctatgtcggcagcggcagaggcaatccaggtggctgggctggacttcggtgcagtagatatcatcttccagcacgccactggccgggcctttgtcctggagatcaacaccgccccaggtctggacgctgaaggcagcgccctggaggcatacgtcaagggcttcagcaaaatcttcgaggagactatctaatggctgttcgtgttttcgtttatggtactctcctgtcaggtttgtacaaccactaccttctggaaggggccgagttcatcggcaatgctgtatcctgcgagcggggtctgatgtactccgctggcggcttccccatcctctccttcgcctcccgtgctgacctcatcgtaggcgaaatctggcaactccccgtgggcgaagcgggggatgaaatgctggagaacctggatgccctagagggttatccgggttggtatgatcgtaccctcaaggatttccgaatcaatggggaacgcatcaaggccctggtgtaccatcaggatagccacatggcgatggatatcgtccaagatggcgactggaaggcacacctggcaaaacgacaaggagcagtataatgaacgaaatgaccgtagacaaagcagtagaagtctaccgcgacaccccgaacaccttcggacaccaagaactacatgcccagaagatgctcctcaaggagatcctgggtcttgtagcttcccagcgacacctccaagactccatcgaggtctccaagattccggaggcctcggacagtcccgagaccagctacggtgggtactgtgacgaatccattggcattcgcttcatgtgggagcgattgaagaaaatcgaggatcgtcttcgggaactggaggaagtctacggtaccttcgtaacaactccctataaaaccctaccgggcaacgtgaatgctgtaccaagcctggttctcaagagtcaactggaggggtaagtgaagaagatcatcggtgatacggcttgtccgggttgccgagctaaaggtggggataaaacaggaaatcacctcatcttgttcgttgatacagaaaagggtactcggttcggaagttgtaaccgttgtggtcactacgaggtcctcgaagagggtttcaaggtaccagagcggagggagaaatctgaagaggatatcatccatgaagtcaacgaagtccttgagtatccaactaaagccctcgatactcgaaagatcagcaaatcaatcgctgaacgatacggggtacgtgttggtctatcacaagaaaatggtgaggacgttatcgagcattactatccacgcactcgcgatggggagtaccgagcgtttaacgtccgaatcctagagcccaaggccttctactaccgtgggagccccaagggcggtgtagaccccttcgggtataatacccttcggcataaggatatgggacacctgcggttagtcatctgcgaagatgaactgtcggctatgtctgtggcccagatcatggagtcgaaactcccggagaagtggaagcatcttcgtcaggcatccattagctggtcctcgggtgttggttctgctggacgggacattgcattccttaaggagtctggtgtccttgagaggttcaacgaggtcatctattgccacgatgcggacgacgaaggccgtaagtcagtagaaaaggtacgtgccctgtaccccgagtgtaagtttgtcgagcttcctctgaaggacgctaatgacatgcttatgcgtaaccggggggatgaggtttaccagatgatacgtttcggcagcaaggtcaagtctccggactgttccgttactgtcgatgaggtatacgctgaggctctggaacccccaaagtggggcaagagttacccgtgggaaggtttaaccaacctaacctatggtcagcgggatggtgaaatcatcggggtaggcggtggtactggtatcggtaagaccctgttggcccacgagattgctgcctggaattgcattgagcacggggagaacgtagggaccttcctcttggaggagcaggtagccatgacacttaagaatatcgcggggaaggtagccaacgtgcccttccaccgaccggatatcgagtgggatgagcaagcctttaaagatgctgctggtaaactccgaggcaagctcttcatgtggaagaataagggtcagaacgattgggatcatatcaaggagtgtattcgcttctgggcagtagccatggatgtgaagactattctcctggataacatgaccgccatgaccaaccacctgagtccttccgaaatgaacacggagatcgctcgtatctgtacagagctcgcagggatggccgacgagctagggctgcgaatcttcatcttctcccaccttaacccacccaaaggtaaccgtacccacgaggagggtgctgaagtaaaggaaagccagttcactggttcccgagctatgcagcggtggtgtcagcttatgatcggcttcgagcggaacaagcaggctgacggtgaagagaagcacgagagtcggatccgtgtaatcaaggacaggaactacggtaacactggcctagtgttcaccaagtataaccctgagacgggtcgcttggttgagcgtgagggcagttacgacgaggtacctgctgacgatgacaccccaatttgattacgtgatctatgaccttgagggggacggactcttcaatgaggtcacaaggctatggtgtgctgttgcagtagacattccgactggggtagtccggggattccggcccgaggaaatggacgtgttctaccgcatcatcgcccatgcaaagttcgtggtcgggcataacatcctagactacgacaaccgggtccttgagaaacttcatgggatcatcatccctccggatcgaagctacgataccttggttgcatcgaggttgacttggccagaccgaccccagggtcattccctgggtgcctggggtagattcctgaagtgtcacaagggtgatttcaacgacttctccaagttctccgaagaaatgtttgagtattgccttcaggatggagtggtcagtcacgcactgttcaactacctcctccgggtactcggcatgacttggcaagagcttgttgaatggaggactgtagattggctaaaaagcgagtgaggaactacaagcgtgaaagggaactggctattcgacgcggcgaaacgggcgtggggtctaagtctggagatgctcagcggcacagggcccgccgaaaggtggaaaagcgtcttggaaggaagctcggagccgacgaggttgtcgatcatatcaaacgtgttaaagatggtggcggtaacggggattctaatctccgcgtccgtagtcattcttctaacgctgctgatggtggtcgtgtgggcaatcgtaaggccaaaggcattcgtaagaaaaaataactaaggaggggccttcgggcccccgaggactcactatgttcaatcgaaagcttagcatcagcaacatcctcagttccttcgacaaggtgctggttaacctgaagacctttatccaagagtcttcggaggaatctgaacggatctacaacgagatcagtctcctgaaggcggagcgtacccaggtcatgcaggacaacctgaaggcccagaaggtactggctaatctggaagaactgctgggaggtaagagtgaagaagtatcgagttaacgtgggattccaggacaccaaggtgttcaacgcagacttctaccgaatcgagttggatatcatccggttcttcgcgggtgattctgatgccaaccccatgaccgtccgagccaatgaaatcggtgctgtccgtggatgggtttctgtggaggagattaacgatggcgagtaagaaggaaagcctggaggatcaggcacggaaggagattgccctggagaaggagttctctggtagctggggtggccccgagatcgatgctgatgacttccccttgggtagtgcctgtggcctagatcccgaggtatgcgagtcatgtcaatagctctacaagtccttgtcatcttctggagtgcattcttccaggtattcctcctgggattgaactctaagctgctccgggatgacaagatcaaggctgggttcgtagtgtcttggtgtatcacgctggctcagtttgcttacatcaaggctgtagcctcctcccacttggatatcggatggtttatcttcgtgtccgggtggggaggtgcccttgggattacctctgctcaatacttctacaagtggtacgacagggtattccacaagaaggcttgacaaatccagaaaagtgtggtataataaccttaaggtgtccggggttgctttacccctataggagatacaaatgagtgaccatgtaagctattccaaacatgtccgtggcaagtacctgtgtaatatggcctctgccctccataagagcatggagatacaaaggactaacatccggaagttcctcagcagtccccagattactctccgggagaagcgtcgggtattcctgagcctccccgctggattcctgggggtgagctacttctcaggctcccacctgaatctgagttcctactcgcctaaacgcaatagccgggtacgggagaaggatcttagcctgtacgatgacttctacatcgacaggaatacccagctagatccgcgagacctcttgtttacctcccaagaagagaagaaatgggagtttcaatacctgaaggagaagcggggtgatgggttcgagctatccgatgaagaactgagcgacgttaaggatatccagcgcaagcttgacctgtcctggtacctggtggaccttgcctgtgagcgtgggtgttcctacttcattttcgactggtgataacatgagcaagatcaagagtgttctcatggagcgggtagatgacttcctgctcaagcaagttgctgtagcgttcttggaacagcaatggcgactggaccgaagcggaaccgtggactacctctcctacctggagggtatctccgacgaggccgtggaggtagtcatagagaatctggcggagcgtcttaagggggactaagtggattggagaaagtcactgttcgttgagcacaaggtagctgatatcatcagtcgccagagtaaacgtggagtctacttccagactcagcgggccaagtggctgatccatgtgcttaccgaacgaatcctcaagattgaccttgaggccgtcccccagatgccgatgatgatcgtaaaggctggggccttcagcaagccattcctaaagagtggtaagccgaaccaaaggctccagtccttatggcagcgtcttgggcatttcgaagtctctggacccttcactgcaatcgagtacatacccttcgatcttggtaagactgccaagttcaaggattggatgctggatcagggatgggttcctgaccaatggaacatcaaggatattactgtcggcactgatggcaagaagctacgtggatccgaccttaacgagtccttgaacaagtacattgaagacctccgacagagtaaatctggacgactccgaatgaagctccaagggatcatccctgggaagactacgatcggagaggtcaagagaaagttagaaaggcaacgaaaggtattgacgactccgaagatgacggaagagtcgatggataccgtccagggagacctgggaaagctggtgatgcagcgaatggtttgggcccaccggcggtccctcttgcaggggcttgtcgatcaggtgaggcccgatggacgcctagaggggagtgctaacccctgtgcaacacctacgggccgaatgaggcaccgtgtagtagttaatatccccgctgctcgttctccctttggaccagaaatccgagggttgttccaggggacacctgatgccggtgaatggaaatggactgtcctccgccgagacattggagagaacgaaagggttaggcccttcactaacatcgtggaggaactcaagaaaggtaagtggaagcctgtaggaaagcacaagatatacgtcccagcgaatcaaatgatcttcgtgggctatgatggtgctggactagagcttcggatgcttgcatcctacatcaataacccagagtacaccaaagaggtggtcgagggtgatgtacacactgccaaccagatagccgcaggacttccaacccgtgacgatgctaagacgttcatctacgccttcatctacggtgctggggatgccaagatcgggactatcattggaggcaccagggcagacggggctagactccgggcccagttccttgaggctaaccctgaccttgctgcactgattgagagggttaagcaggaagccgagcgaggttatctcgaagggctagacggacggaagctaaccatgcgacgttctgagtctggcgacgtgatgatccataaggcattgaacaccctcctccaagcggcaggtgcaattgtcatgaagtgggccatggtgctcctagatgaacgggtccggaggttgaaccttcgggcttggaaagtcctggatatccatgacgaaggtcagtgggaatgccacccagaggatctcattgcgctacgtggacagatggaaatctgtgtccgggatgctggagtgctccttggggttaactgtcctttggctagtgattccatcgctggtcgctcgtggtatgacacacactgatacatctgggggttgactttcagccccctttgtggtataataccttcttccctacgagaggtttaagatatgtctaagaaagtatcccaacgattcaccttcccggtagcgaagctgatcttcccctacatcgtaactccggacaccgagtacggtgaagtctaccaagtaaccatctgcattccgaccaaagaagaggccgacgatctggtacagcagatggagtccaaggatgcccgactgaagggtaccatcaagtaccaagagcgtgacggcgagtacctgttcaaggtcaagcagaagaagcacgtggattggatgcaagatggtgagcggaagtctgctgtaatgaagccggtggttctgacctcggacaacaagccgtatgatggccccaacccgtggggtggctctactggtgaagttggcatcctgatcgagacccaaaagggcccacgaggcaagggtactatgacggccctgcgactgcgcggtgtacgactccacgagatcgtatccggtggtgacggtgaggacgatccgctgttcggtggtgccttcaccgaggaagagcccgaggatgtattcgacgaggtgttcgatgacgaagacgctcctatctaaggggttggaggatcacgaggcgggggtatgcccacggggctgcccctactgcttaatcgaattcgaaagagtgtggggtgtaagggtggtcagttctacagctgcatctaataataaagtagaggtcgatcctaatggaatcaagccgggtgagccgggcgctaaacttgatagcggcaaggtggatgttggaatcatcttcgaagcgttcccgagggctctatatgcagtggcacaagttgctaacttcggagccagcaagtatagtcgcgggggttggaggtctgtcgagaacggagtccagcgatatgatgctgccttcgggagacacctccttgagcgacacaagggtgaggctttggacccccaaagtaaactaccccaccgataccacgaagtgtggaacgctctagcatccctggaactagtcattcagcaagaggaggactccaatggaacttctgttggatccaagggctaagactgttcctagcaactactctgtaaaaggcgttgatgtagacctggggcttcccccaggctacagcctaacggaggaagctatggacaaggccaaacgtcaagagagtgaatattacgactggaagggctacgaagcactggttaatccggtagtagagcacccagagtatcgagctaagggtgaagcctttgccctccgtgtattctgggaagagaagctcaaagagtctcaggtcgtagaagaggtaacgtaatgattgctggtatcgatggtgacgttcttaggtatgagctaggtcacgtggctatgtcgaaggaacacatcttcgatatccaggtggagaagccatggcctgaagaggaagtccacaagctcgtcgatgagaaagtcgaacaaattatcaaaagggtgaatgcagatgagtgtgaaatctaccttactggccaaggaaattttaggctggagcttgcgaaaatcaagcaatataagggtactcgaatcggtcttgaaaagcctcatcactgggaaaccgtgtcagccagacttaaggacaagtggggagcaatcactttccacggtatcgaggctgatgactggctcgggattcgagggactgaagagggagataactttacagcgtgttctagagacaaggatatccgccaagtcccaggatgctaccattacagttggccctgtggagattcccagccggagttgggaccgtttcaagttgatggtcttggaagggtctccgcttcttggagaatgtatggcgttaaaaagccgcagaagtcatggaagcttgagggcaacggtacggcattcctctacgggcaactccttgttggtgactccgtggataacataccaggcctcccagggacgggaccaaagacagcggcagatttgcttggggagctttctagtgagagagatctcttcgcagcttgcgcttacgcctaccaacagaagtacggagataattggaaagagtaccttctggagaattttcgtctcctctacctcattcgggaccgctcttggcttgatattcagcagtccggtaacgagtatcactgctcactgaagaaacattgggagattccctatgacgatgaagaaatattctattgaggaagcacagaaaatctgtgaaggcctctttgagatccttgagggtcttaactttactgactacaaggtcgctggtggtttccttcgggatgcagacaacggggttgcacccaaggatatcgacctctatgtccgtaggccatacgtggaggaccccactgatactcggcgtagtcgctttggaccacggttgatcccctgtgatgacgataccctagaggtagaggtcactcgattctacaataagataggacacaagaaggtccggtgtaggactggggataagcctgatgggtatcctgcggggtttgatgtgtgggagtccattggtgttgacctacccgtcaacctcgtcgtgactactcactcccacccagcagagttcgatgtaggcctatgtgagatcgcatgttggcctgtaaatcggtttggtttgaagtctcaaatctaccgcacaaaggcttatgagtttgataaggaagagaagtgcattaccctgaaccgtcttctggaccctctcctggatcattctcaacccctaactgacaatcaagttgaaaaggttgtctctcatatccaacgtatcaaactgaagtatccggagttccgggtgtgtctgggggattgggtttggctgctgatccggtctaactctgtcctcactgaggggacgctatcggttgtccgtaagcttcaagaaggagggctcattggcaaagcaggggagattcttcagacccaaactgaagtcattgattgggacgaagtacgacagcgaaaccgagaagatcgtccacgagacgatgccttggatgcagttcaagccggagccggtaccatacgtcatcgagcacaagtacaagccggacttcaaggtatcgacattacaaccctgtggatcgacgaagcatctgctggttgaagtcaaggggtacttccaggaagcctcggaggcatctaagtacatctgggtgagggaggctctccctccagatactgaacttgtgtttatcttcgagcgccctaacacagcttgccattggcttagtaagcgtaaagatggcacaaagcaatccatggcggaatgggccgaacgtaatggcttccgctggtttactctagagactttcaaggagtccttccctaatgagtaagaagtataatgaagacactctcgtcattgcggacacccaagttcgatccgaggtcaacatcgatcacctcgggaaccttggggagtggatcgcacgtaaccgccccaagcgaattgttcatattggggaccattgggacatgcccagtctgtcaagctacgaccgtggtaccgctaagatcgaaggccgtcgagtcctcgctgacatacaagctggtaatgatgcgatgcgagttctgctggatcctcttcgccgcctacagcaacaccaatcgggttgtaagaagcgtatctaccgaccagaaatgcacttcttcatcggaaaccacgaggagcgtatcaagcggtatgaaaactctaaccctgctctccaaggttttattgggtacgatcattttgatctgtccgattggattgtccatgatttcctcgacgtgggtgttatcgaaggtgtcgccttcgcccactacttctacaatcccaacagtggtcggccatacggcgggagtgccgagcatcgcctcaataagatcaagcgcagcttcgtccaaggccatgagcagggattcaagtaccacatcgaggcagtaggcaagaagcgaatccacggattggtagtcggtagcttctatacccacgatgaatcctacaagggtcctcagggtaacgaccattggcgaggcgtagccctcctccggaaccacaaggatggagagtatgacctcaagctgatgagtgtggaggagttcctgtgagtaagttcttgccagacctgtactatattaagtctgagcatgacttcggtcaacgggggttggcgtttaagacgccgatctccgcagaactctggctggatatgaagtttgggaaagggggtgctgaggaggggcttaggcgagggatgtattccatcgaagtcctggagatcctctacatccccagcgttcaccttccggatatcttggggtaatctatgaaagaccgagtaggacgtaaactagaggtgggggacaacgtagtcttcctgatccacaggaacacatcctcccacctagccattggtaccgtcgatgggtttacccccaagatgattcggatcaaatgcccgaccatgagttggactattgacgctgagtatgttctcaggagcagtgacaaggtggtgtactatgacaaaggctgaactggagaaagcacttgaagaggcggagagcgctcttgcgaaggctgaggcgaaggccttttcctttgaagagttggctgaagaagctaaaagacagattgaattcctcgaagggatgctagacctagtagacctcagggcttctgtattctacggagattggaggggttatgcagaacgatcaaaaagttaagggtggattcccctggacctaccttgcggtggcagccttgtttgccctactggtatatgtaggatatagctgactgatgttactcctgaccctgggagaaatatcaagactcctcatcgaagtgttatcttgggcaggttccttatgaagtatcgagtgaagcaagtggggaagttcttcttccctcagtataagcaatggtttcgttggaggaatttcgagcagagaaaacaggggatggggatccactgtgtcagctcgtatctagagtcccttcctgtagtcttcgagaacctacaggatagctgttgcttcatccgggaccacatggacaggatatccgaagatactcctatctaccaccccgtcgagtaaccaaaaaaaaggccccaagggtgttatcccaaggggccttatctttagctccggagagcgttcagtagtgtattgaacttatccacgagtgcctcgtgagtatccgtataggaagccacggggatctcagatatcttatcctggatggaagccgcaatggcttcatataggccactcacttcgctagccttgggtttccagttacctggtttagcctcgctagccttagcacctacctccagaagcttaggcttcccctcaatatcctcccacttcactggctcaaaatctttcgggccaatgacttgtcgaagggtcccatcttcattacgtacagcaatagccttcgattgaataacgaatacactgttgccgtcgttgtcagcgatataacctgccataatctaactcctattaaactgccgatttgaaggtacccacggtaccaccgaaaggatacactcggacagtgcattccccaaattgaccatcagtttccgtctgattacccttagcataccaagccttgtccggagatacagcgaacttgatcttagctgtagacccttggataaagtggaagtgacaacctggggttacatctggaccaagagtcacggtgatatccgtggtcccttgagacatgaagaaccaaccagactgctgcttagtcagggtaatgttctgagtaagctgctgggtgttgacgacattaccaccaaggtgagtagtcacgaggcggggatccaggtagttctgttggagagaaccaatgttcttcacaggaccacgcatgtcatcaaactgctcgataatgtaatccccgatggtaccaccgttttggtcatccgggccaatcacgtgcttaatagccacgtaaacacccaggtgttcccgacgagggtctactcggaattcactaccgaatgcagtaccgcaaggtgcctcaatcatggtctcagagaatagaccaatcacgtggccagggtcacggttaccacccgaggtttccccaggagcgcccacgagggtgttgaccagaacagaggggataacggcctctgaggggttctccgggaaggaggctgtaacgttctctactaccagggctgcgttctccttaactgcataatagtcagggaggaactctgtagcggtaacgatattagtattcacccgaccggcatccgaggtaatctcaatgagatcaccatcggaattacgtcggaagacttttggcacatcaaccacgtaggcacggccaccaggaccagcggcatcagtttctacataagttgccatattacttctccttaaagatccagagcatcttccatcgcttccataccggcaacgaaaggacgtgctggagtttgcttatacaagaacttccagatttcacctgcggatgggtcatccccaaacgaagcgatagttttaccaacagccatagcaccctcagttgcagcaccagctactggacccaatagtacctctgcgggggtagtccctcgacggtaacccgtcagcatgtcgtagatcatagaagcctggagtggcatctgttgcatcactacgtccatcatccgttgttctggactacgggtatcctctcggctagacccaccgaacttagccagttgacgaagctcatcctggagataccccagactcatcatcagaccaagagtgaaggctacacctgcggcacccataccagcgttggtccaggaaccagcgaagtgtgggctcattcgtctacggaacatcggtaggatgatgttaccataggctgctgggtaacccttcaagagggagaacatctgaacgtttccgttgctcatccacataggcttatcagcgaaggtgggatcgaggactacctgatctacaaaccgacgcatggccaaagtcttgacgttgttagccatcaggacttcagatggggtagccggggagatcaacttgagggcatcctgctggctaccgatgttaacccccatttcccgaagctgagcaaccttcagagcaccattggcagaactgaaggggagacctgcggctagatccatcaggttgttctgatagaccctcttggcagtctctgttgcaaagattcggttaacatgggttaggatggacaagccgttaatgaggaactgaccacggatcgtcttttggatagtagagttaaacacctcagcaccaacccgatcagccatcagagaggtagcagaggccagggtgtggttcatatcactcataaaccgaccggtctcagactttggaaccccactgtagatcctacgggctgcttgtcttactacttcacccatggttgggagtacagccccaagggtaggcataacccccgccttagcgaagggtaggctgaactcagttagggtagagaaccctgcgaggggtagtcgggagagcacgagggcacctgacgtaacagccgccagcttcttaaggttggggtctttgatacgaccgtgcataccattgtaggcatccactaggtcatacatccgatccacttcttccttggtaacccgcttaccagcccgttgagcctcagctacagcagaagcaatcttagcgttagccttctctccgttgataccaaaccgttcggtaaaggcaatccggtgggaagccccctcgaagtaatctcggatttcctggagacgcttcttaggagtatcattaagagaatacttattgaggatctcttggggaacggagccaaaggcccgactctcttccagctgaccatacttaggtaccgcatcactttgggcaaaccgtccacgtagagtatctggatcaccctggatacggtaacgtgggtctacttcccaagccccggtctgctggttctgagtaaccaatcgattaacctcaggggcagtgttaccacgagtatcatccgagacttcagccagccagttagttacagcatcttcagcagcttgtcggttctggaagtaaggagtgatatcgttcaggaactctggggattgaaccttctcaggggacagcccaaagggcatatagttggggatagtcccaacagacatgccaccacggttaacagcctcattccttacgtcatccatcaaagaacgtagacgggtagcctcaggggtgttgacaccagcggatgtatcagcgataatcctatcgatctctttggaagacttaccctcaaagatgctatctagttcagagttccacttacctgcctgtagttcctggtcctcaaagatagtcttaccagaggcccgcttaccactcatatcagcacggaaggtctcagagaactcacgagcgataggggaggccttagcgagtggctctaggagcgacgtagcttcgtttcctagggcatcccaagccttcttgaccgtacccctaggttcaaactcagaagccttaggaggtgcctcaggggcgttaggatcaaccacggcagatccagcggagtcctgatgacgacccagggtgtccatccccgaggatacagcaccaccggcagtacccatagcggtaccagtgaatgcagcagtcaggaggttatccatgaactgctcaggggtttgggtctgcccaacggcatcatacgcgatagtgtcctggagggcctgctgggcaccagaggtaacaccttcagccacaccagagactacagcgtgtttaccagcttgggtaactgcttcaatagcagtctgcttgggtagaccggactgtaccagcatatggtatgccccgtctttcccaaggtgcttcaacaggggaccagcgataaccccagcccctgcggtatccagggcagacagaccagcaccacctaggactgcggtccatgggttgctttgatcggggtctagttccttcatctggttactcagggcacctacgttgatacccatggaactcaggaaggaaccaatgagcgctccacccatacgacctggggccccaaagacagagccagccttagcacctgcggcaccaccagcaagtacaggggccatcgaagggagagcctctacaatgttattctttaggaacgaaccgatggatgggatatcttggatatcagcgaaagaccgaacatcgggggttccgtactgtgacgcttcctgagcattctcctcggccatctgtgtgccgtagtctttcaggtagtcactgccagtcaattcaccgagggtagcaatggtgccaccgatgttagactgcatggtatcaaccccgcgaccaatcgcggagctaatagaattagggtcagccggagttactagggcactcaggtctggggcaggctcaggggtagcaggggctacttcttgaggtgcctcttcgataacgtctggctcattcaaagaagccaactcagcggctacgtcgaggcctttgacctcagccagttccgcatcaatagcggctttcagttctggagaaagagccataagtcaccttctatttgctggaaaggaaaataggctcttaagatgtatcctaagatactaaccttcttagtaagtaagtcaaaaggaagaaggaatcttaagagcctataggtctattatactacattttggatactttgtcaagtaccttttatcgggttggggcaggatagtccaaatcagcaccaaaccagttacccgtaggttgcaacttagcagcctcccgctggataatacctacagggttagcccccggattagcccgaagttcattccggacagtctgggcaatagcctgttgggcagtcttactcaacttcttgccacccagtgcttgagagccgttgacttcactcaggatacccagggcgtccttagtagtgacagcttcaccacgggctgccttagctgctgcttggcgtgcctgggcgctaatcttagcggtcttgagacgtacagcagaactaagctgggcattctcttgagacatatcctgaccccgacgagtggtttcagcctgtagctgagcccgctggttagccaggtcctgtccacgctgggttagggcaaggttggctcggccccgagcagcctcctcagtccacctatccagaagaccagtgttcttacgagaggtttccttaagggtgcccttcttgacctccaggtttccttccccgagacgtacatcagcagcatccttagccaccttacgctcaagatccttctccttgatctgacgatcagcagcagcagcagccatcttctgttggttgataccagcctggaggttacgatcaagttgacgctcataagaggcagcgaacatatccccagctttaccagtcttgtctagggccgaagccagaagaccagtaccaatgagagcgtaagatacataacgagacaggtcatcattatccatagtcctcatctgggtcaactcttcgttcacccggttcttaagctcttggggtttaagctctacaccctctctctgggcatcagcttcaactacagcctgggccatttcaggacgacttacagcaccagtacgaagaccctcggctgcaccctgttggatgacctgacgattagcctcctcctcatctgctacagcagcaccagccgcagaggccacctctggagcgatatcaggctcgatagagggttggttaggggttaccccataggacaggagttcagcccccgtagggcctcctgtggcgttctgggctgcacgttggcctacaccctgggcccattggtttgcctcttgagcaacctcgggacccatgttctccacagcctgactagcctgagccatttcttcagcacgagtacgctctgcatccccaagttctcctggggttacagccgcttggataccaacagacactggaccaccaaggatacctgcgatacgacccagggcaccacgaccagtagcttcagccgcaggggccacctcttgggcagcccgagaccaacggtcagggttctctagggcagcctgagcagcccgtcggacacgatcagggattcctgaaccctctacagccagcggcagtttgttccttgcgattcgcgccatgtccagttcattctgagcagcccgaaggatgctagggcgaattggggacccttcataagccatgctggccataggattaacctccaaatagtttacttaggattccggactttggctgctgtccgaaaccgagttcttctgcgctaggaaggattcccattcccgctcccatacctgctgcaacttccgctgcgcctcgttcgcttgctcctctagccccctcgacagaaaagcttggtactgcttctgaagaaggctcacttccttgcccgaggagagccgcgagtgctgctccgcccattgcgccaagagggtttgattgtccactagactctcccgattgcattgtcccaaggtgggtggggcggtaggagccttgacgactcaactgaacccgctcctgcccaccacctatattctgtcctatagcgaacagtttctgagggagggaggccattagaagagaaggccccctaccctaccacctgcgttcataccaacagaagcaccagctggaccaccgaagagagcacccaaggaagcaccaccgagagcacccagggcagacccgaggccaccaccaccgccaccacccgaagaagtagtgacattggttccccccatatcgccggagataagctccttataggcgaggaggtcgttgaggctgacgttattctcataggcccacttctgtagagccccgttgatttcctgctgctcctggttctgaagcatgctaccagcatctacctgcatggcattaccagagccgaggcccttagcaatagccgacaggttacccagggtattcaacctattctggttgtaagcctgctggtcttggaaagccaactgggaggcgttattctgttgactctggagcaatctagcggtagcaataccttctgctacacccgctcgggaactcccatactgaccagcattagtggcccctgctcgcaggtctggacgtaccgtagtgtcgaagtcccattgcatctgctcgttggctgcaccaatggcgttcgccaagccagttttgttgggatcgtaaggaccaaggtaatcagccagagagctaacacctgagctacccaggagagactgaagggcacccccgagaccacccagcccttcgatgccaccaagctggagagcattttggtcagccaccgggtcaaagttcggatcgcccccgtaatttgggtcaaagcccccgttatgtagccaatcactggcacccgagagtagttcattataattaccttgctgatagggtgtagagacagaggtggtcttttgcttcttactaccacccttgtaagcccgagaatctagggcatcctctacatcgaaccccatcagacgctttacattaaattggaggaagttcatctggagttacctcacgatagaaggatacggagtcttcggtgtacccgagtttctctagggtaggcttccagccccgacgaccttcgcattggataaaccgacagttaactcgttgggcgaactgtccgaggaagtcgtctacctccgagtaatctaccggggtttcattcccaggcatcttaccactccagaagaagtgaaggatgttacccaggggtgcttgggacacttgaattacaccagcgtagccactctcttcttggtagaagacataggcctcgtagttaaccaaggagtgaaccaagtgttcgaagtcccaaaacttaccgaggtccgtcctgttgaaggctcgggccagagcagggactacggtaggaagcagatcgatattctcacgagtaatcaaatgaatcatggagtcaccaccagggggaatgtgaatgtacccccgaagactgctccttggggaattccttggattagaacacggccattagctgtgatcttaaacattgcttggttaaccaccccagtctgaatagtagccctgttgagaacatcaaagacttgatccaacggaggtgcgttagggtcagacggaggtggataagtaatagtgctctgcgctgggatgatgctggaataggccgggataaacaactcagcaggaggccaatacgcctggggtagatccaggacagtagccccgttagtgtagttaccaccggacataagcatggttatccatacctcatccttagcagtgttcatcctataggcacaggtacccatcggctgatggttgttctgtggggtaaataggataaaatcccccggcaggtccttgggtttagtacctgcgagtctccattggttatccaagtcgtaatgatagatgccggatactggacctaccactccgggggcaaaatactttacagtccctggcttgagcttcttagggggctccatagagaccccccagtagccgtcagctaggtcattaagagtctgtccaaccctaacgaattcttcattaaggaagggcagcagttcctcctcttcctgtggtggaatcgaagggctgtacttttgactcatcgcatacctgccttcggggccatttcaatagtgtacccattgaagtaccaatccccctccgaagagaagtcgaacttcaaggctatataccggcccacatgtttagtgtcaatcttatagtcctgaccaatccggtatgggtaagggcctttccatcgaataccagaaccttgtacctgagcgttacccacccagatgttacaagtaccgttaccagtaatgtgtggaatgatggcactcactgtcttcatcattcgatcatccccgagatagatatcggatctctcaagggtactgacgaagttctgcccagagaatgtagagttattaccgaagaggaacaacttcttatcctggaaagacgagaagatcatactggactttgctgggttataagagccttcaccccagaccgaagtatcggtatcccaggggttggggtcgtcatcccagaggttagacaccttaggatcgatgatcccgtaggctccactgagaacgttgggaaggtctcggatactccaagtgttttccttccagctccagatgatagccctgtcgcagtgcttacctggctcagacctagtggaagagtagcatacccacatttcagtattcacgtggtctgcaagtacgaaggtccgttgatagttgtcagggttaatatccgagaagaagaacttacggacctgggcatcaataacagactgcttctgcacaccgttgtggacatatacatcaccgtgacctactacaaagtggttaccatcgaactctactgcacagttgggcccaaggatacctacgtcgttaaacaactgctggaactggaagatgaacaatccaccgatataccgcatggagtatacagagtcttccttgtagatgatgaaggagtcacgaagcttcacaccatccacgatagcaccattggtatcagccaaggtgttctgaccagcatccttagtagggtccgttgggtcccaagaggcgggtacaccaccagcatcagccgaggtactccaccagaccatctgtggcatttctacagagttacttgtagcgtttaagccaaccaggaagttcttaaaagacttaagcctcttaaaagtagtattcgctgggaagttaggaagtaccctaaaggttgattctgacggtggaagatgatgaggggggttaaccccatcgttagcaaagattaccccgttgaacgaccctacagaccacctgttagttatactagcagcgtaaggtcctggggatacatcgatgattgtagtcccgtcggctagatacaacctttgttcagaacacaggagccaataggggatgttattccggatgaaaggaaacatatccaagattggggcctgggctgtatcaaagataggcgtatggcccagagccttctgagccttgccgttcttaaaccggacgttgttcccgaaggaccatttctccagtggcaggtcagcgggggcgatatcggtcacaatccccgtagggttcttgacctcttgtctctctagggccattgtatacctcagttcttaatgatgaagaacacagaacagaacggtgggatattacccaacggcatgttgatctttactgcatggttgtgagtctgaccttgaccagcaggaccagtctcaaggttggtattggctgcgttaccagagcctcccgtcagagcacccgagtcacccgcagaaccggtgagggtagtagcacccctagttctccaagtgtgggtgtgtgatgggatttgggctagggtaagggcagtcccttcagtgaacccatcccatacaatgttagcgctaccgcctctagtacctacagcctgggaagatccgtcgataccccaggggaatgcaccaatgaggttggggactgggataccattagaggtagtccctacaccattacaaagcttccaacctgctgggatctgagccagtgacccagcccacatgataaccatcccaggtttaacatactgggttgtatcagcgactgcgtttagctgggctgccgttacagtgacagcctgagaaatattggggaaggtattcttaatagcactcttaatgagtcgcaggtggtcatccccaaaggacttcagatcagagccggtagggttcgtaggcaccaactggttaatataagttgcgacctcaagacccattcttggcctcctttatctcttctttcatttcttttcgggtgacataccgctccccaaagattgccatagaaatctggagatcgctcaccgcttgggtgagcttctccgtggcctggatgtttctttcaagcagagcctgatgaacattctgaccgacaaccgaggaacctaccgtcactaccgaggatacgaccagggcactgacgatgctgcccaggttatcagttagaagttgcatcctccgtcttctccttaaggttgtcttccaggacttttacgaactcgtcgtcaaccttagagttagtcttctccgccagggccttagcaccagtcacgatggacttagcgattaccttggttgggaagagggtagccagaaggttgattgcgagggtttttagaaagataggcatctgaagctccttaacgttcaatgtctttgatcgccagtcgagtgctagcgaagtcagcggcattctcctcgttctggagttccataacagcccgttcgagtttctgaccccagaactgggaccgagcttcatccatggtgtacagataaatctgctcaaggacaccatagagataaatctgcggatacttcgtcagagcccaagtcgttgggttagcgaggcttagctctgggagtacagtccagtagttcacaatgaacggggcaccgtcaggaacaacggggaatactcgccagaagttacccaaccgagtgtagtaggttacaccctgaggttggtagttgtagttaacatagtgggtgaagatatcttgggtgatgtactgaagagtacgcccaccgataagagagtcacccgtgatagaccttagagcaacaaagtgctcagggatctcaatgccaccaccgaaggccatgagggtttcgaagtgttcgttctccctcactcgtagcaatcggttaagacggtcagtagtattcccaatgaacagcatcagaagttcttgggtaaggtcctgacggtcagaccactggatagcggcaatagcgagatcagttacgttgttgatcgtagccattcattacacccgtgcctcagaggtccgcatccgatagttatctcggtcattaagccaacgggtaaatcttgcggcatgatctgggtcacaaccaatcaggttaagatcgatgggaccaccttcagccatggggcgattccgtagtgcctctacgacaaccaaggggatacttgctaccttgcgcatattatccttacggttgctattgacacccgagtggcgctcttcggcgttagcggacaggattgactcaacatcttgagtatcctttcggataaagagcccaaggtcttcgtcaattgcataagtcgattggatactcatgatgtacctcctagggggaaacaaagggccccgaagggccccgttgggttagacctgggctacaacgtctcggatcagagcaccggacttctcgttgtttacacgcagggtgtactcaaccagcagctggcgcttctcgctgtcaccggtcttagccagttcgtgctggaagaacggacgcaggtagcagagagcgtgcatcttcggatcaaagatgaacatggtgttttcgtggaaccagcggttggcacgaatggtgtacttaccgaaatcactctcgtagacgtccacggtctgcgcaatgcggttgtccgaggcatccagggtgatctcagttgcacgacccttcatgttcttgctgatggccttcttgatcgagctagaagtctggatcgagttagcctgaccaccgttgcgccagatagcctcagaggcattcaggagcatgtcttcggtcagaagacggaggtcaccagcggtaccagtgtcggaaccatcaccagttggcagggtaccgttagcacctaccgaaccgttggtcttgtagtaggcaaagatgtttgccatctgacccggagtagtggtgttacgctggatcttagcctgaggggcaccgaccatggcgtattccatgtccagcttcagttccttcgacttcttagccagctgatacgccagttcgttcttacgaccagccttcttgaccttatctgcggtaccggtgacttgcagggtctcgtccgagatttggcagaagttgttcagcatggtggtgaagctaccagccttgatggttgcatcctcaccttccactcgggtgttcttacccggctggcggagttcatcagtctgccactcgtgggtgatagcggtagctacgcccttgccgatagcagtcatgaacggggtgtcatagggtgcgatgttgtagatgatatcgataaggtcttcgcgcttaccgttgatctctacagtcgagacggcattagttggagttgccataagttacgtttccttctattagaaaatatcgagagaggagaagagagcagcagcggcttcaaccgactggtctttcctcagattagcgcgggcagctttaacccgcttagaaccctctgaggcttccgccctacgagcagcaggcttaactgcggcaggtagttcagtctcctccttcttctctagggcagccttgcgacggacctgagattcagcccacttacgtgctgcatcgagtacagccagttggcgggcatcagatatccctcggatctcatcctcggagtatccaatcgatttgccgtaggacacgatcttgtcaccccaagactcatccgtagtcatttccgggataagtttcttggctagctctgtctgacgcttaacgtaggcagagtggacaatctcggctcgcttctcttgcatagccttaatgtcgttacgacgtttgataagagcctgggctcggtctcgggcttccagggcttccagtcgaagggtttgatacttctctggatcctgggccttaagctgctcccagtttacattgtcatactgattagcaccagcgatagcggtaacagcatactgctcaagctcggccagtagattagagcgttcagcatcaagctcttcgaacttagctgcatactgatcctctagttcagcttgtcgagttacgaactcttcattgcgaaggtagccactcttaagctcttcgaagttaacctcgtagacttcatccccaatcgggatctcgaagagtttatcctcaggatcttcctcggactcaacctcgggatcttcctcagaaccctcttcttcctcctcggactcaacctcttcggtgtcctctggagtaccttcgtctactacctcttcctcctcttcttcccctacgactttaccatccacggtttcatcaccgggggccaggaggtcatcacctagcaaatctccgaaggcttccgctgcctcgaattcatccatgccttgattctcaaggtccattacttatactcctttagagtgatcgaatccaggatagcctgaatacgaagttgtacgcggttgagggcatgtagttcgtggtagatggcctccctggattctgaatccttaggtgccgtggacttccactctccctcgatctcttcttggacaatacggaaaagctcaggaagaacgttctcacgtaccatctgttgtgccgcatcagtcagcactagactatagccaaaacgttctcccaagattatttcctcactgccttcgagggcttcttagtctcaggtaccttaccgtctccgatataagcagcccgagcctgagttgcctcaagatgatactccgcttcgttacgagcccgttcccaagtgaaacgatcacgctcaagttgtagttccgcttccttaagggccatttctctctgctgaaggacagcctcttgcttcttcagttcgatctcagccagtcggatctgagcctctacctgcttcatctgggcctccgcttgcttagccatagcgtccgattgggcacgttgggcatcagcttgggctttgatatcttcaggcttaggttgtgcttccttctgctctctgatggccttagcccgttgagcttcaggagaatccgggttagtccagaagcgatccgggtctttgtacccagcgttctctgtgacttccttaaggatattataaaggttctgctcagagacaaggaccccaagaccaccaccccctactacagcctgggccatttcccagatacgcataaggtggagcatctgctggtctttgttcatgtttccgataccaacggtaaccgtcaggtcggatctctctcgccagttggcagggttaatagcaacccacttgcctcgtagctggaagacctcttcctgattctggtacttgatggcatggtcatgcagaagttggaacaaacgcttaacaccagtctctgcaaacatccgggcaatcaggtcaatctgttgctcagcagcagtcatcaactggttaacactcatagccgcttggttagagtgtagggtgttttggtctaggcctcgggtacgatcagtgatgcctgtccgcttacccctatcagcctctagcctatctagcataccgtagacttccccagacagctgaggggtctccagaggcatgatagagttcatggccttaacccgaacgatacccgctgcctcgttggtcagcaagtcttcgaggttaacctggccatccaggactacagatcgcccttggttggtccggtagatattgtccatgatgttgcgcatgagcaccgaacggatctcttgaatgtctcggatcttatcgtagacactcatcccgtggaacttatgggcaattcgataggcattcaggtcagcgaagggacggcaatcccaaggctcgttgctgatgatgtagtcgcccacgtacaggatacggcgcaactcagagataccatccccatctacgtccagaagggtgtagcactcggaggcccatacctcacggttggcttcagcatcatccccagagttgtactggagttggccagtcatatcgaagttatcacgtaccaacctttctggctgactatcagagaattcatactcatcgtatggaagctcatctagtacatcctcgggaacacccaagagcctcaggtcacttacggtatacttctcacggtgacagaggaagcgtgcatcatcaatgcaggtagccaaccgatcaaccaggaagttctcaggcttgatacaggtgactttaatctctcgcttcttcttgtccttgcgaattttaatactgtaggttccatcctcgtccacactctgtgctagaatctcagtgtctggatcagccaggatatccgctaccatttcctcagagagaccagagaatcgttcgaaggtagggttcaggacctcttctacatagacctttacaacaccggtcttcatcatcagagtgtcttggaaccagtcgaacattaccttgaacccctcgttcttacgcatgaagaggtagttcacatactcagtctcttgctctgcctgttcaacatcttcggcagtctgaggttcatacttaactacttgaccgcctgacgtgaataccttcataagagaaggcataatccagtctacagtctcttgaacgtccctagatacaatcgcggacttcccagggcgctcgttaccgaagggctctccgaagtagtacttcagggcctcagaacgctgcttggaaagttccgaagagttgaaatcaagggcgtcgttaacaagttggtctagatgacgaagtacctgttcatcatccataggcttaatcttgcgacgacgcttagccattagacaatactcccaaaccaatcaggggtcaactttgcagtatcactcctgtagtatccactgtttcgtacagcaccaggacgtgcatgccgggaagccatcaacagggcataccgggtagcggatatcatatcgtcgtttctgtcgataatctttccgtcctttcggtggtacattttcatttcttttaggaagttcgtacacgtattaaacaccttcagatcaccattctccatacgggtcaacatccagttaacaccgaactctacagagttacctccgtgtttaccatcaggacccggtgggttactgaagggctcatacactacattgaggttgtggtcatctttaagaaggtctacgaatctacgaccagaggttgctccatcgtgcttaaaggcatcgtgggggacaactaccgggatctggtgaccacccttcaggtagatagcatcagcgtgcatcccaagggtctcaccactctcacttctctcatcatagaggtagtatttgtctttctcagggtcccaggccacacaggcgatagcgttagggtggtcaaacccgaggtcgataccgatgatcctatggaagtgatcggggatctggaaaggctcacatacaaacttctcttccagaatggggaagactacaccagatccgagcataggaacaccctcggccctcatcctgcgttctgctggagagtataccgagagtagctgctctttaacttctggactgaggtgtggagcgtcttcccagcttgcatggacaaggaactgaccaggtttaagatcctggaggaagtccttgacgatctccgtcagaccatgctctggggtaaacgtcagatatacaatacccccagtagtagccgttcgggttacacactgggtataaatatccttggggcattcctcatcgagccagatgacgtcgatggcagtacccatgaatttgtcctgggacatttcgtaggacttgaagataagggacgagaggcccccggaggcatgcttaacaactactgcttgcacacatccgggtttaccttccctacgaatcgtctctacgatatcttccttggggatcatccccgttccaaaagcctcagggttcttccagtcacctagtagttcggactgaagaatatcccgagtggtatccgtggagatccccgccgcccagcagttcactggacgatcatactttctaccagtccaccactcagggtagcgaccggtgaggtggcaggccatgataaaggccccggtgtaggttttaccacagcggttaccagtcatagccagcaactgggcgcagttcgaggaagctgcgataaacttctcttgccacccataaggagtatactggttcatgcggaaatacttctgccgctccgccaactccctgactagattccgtagccgctcttgggtatccattagaccaccttgtcacctactgttgatgcacgccacagggcccgttggaggtcccttacgcgcactggagtttgtttagcccagaggctatcctctgcctcccatgcagcctcatcaaaccttccggatttcagaagattccacgtcttgaggaacttcttcgtccacgctgtacccagctggaaattgacgctaacgagggcatcgaatagctctggagtgcaaaaaggcagctgagatacttgcccttgggctgcatcataggctgccttactatccttctctagccaagtctctgctcgtgctagggtaatcggtccgtcttccccagggagttgcaagtggccataaccaccagttactttcccgagggagtctttgtaccactttagtaccaacccctctcttcgcttgattagatcaaggtacttaagaggctgggaaagaaatgactttagccaactctggctcttcgtcgagaattcgtcgaacttcactgattagatcctctgcggacatatcttccacattcttagtagtaagctccagtttctgcttagctccgaagcctccacgatccaggatatcctgggccgccttgaggcgaatcccacctttctcttctgggttgctagcgatctctacaaccacacgaagcgccatagggacgtgactcccgatgcgctcagagataaaggcgttgatgtattcggcatgctttcgatggtatgcagctacgttgcgttgcgcatgattaggagagaacccagcggctatatatgcttgggttttgttcataccatctgccagagcctcacaatagaggtctagcttttctttagcagaaagaggagcagccccaatcgagacaaccttttcgtctgacatacactgctccgatctctctaatggtcaccccggtaggactcgaacctacgacctcctgtgtccaagacaggcactctaaccaactgagctacgaggagttaatggtggtcgaaggaaggaattgaacctctagcctatggcgtccgggttacagccggatggtagtagccatctacctctacaacgacctttgttggcgacccatgtcggattcgaaccgacggcttctccctagacaggggagcactctaaccgctgagttaatgggccataatggctgggacggtagggctcgaacctacgacctgagagttaacagctccctgctctaccaactgagctacatcccaatgaactcttaaggttaatttctcaaccttataatactattatatcacactttttctattttgtcaagcccctttttcatcttttttttatttttagtcgcaagacccggatttgttagggtttttgctggaatgggaaaggggttcttaagactatcttaagataatcttaggataactaaccttcttagtaagtaagtcaaaaggaagaaggaatcttaagagcctataggtctattatactacattttggataccttgtcaagtactttttgatatttctttgataatcacatgatttacccgacgaacggtatgatagggttctataggtataccctaagactacccttcgtaggttgctggtaaggggtaagtggcataaggttcccttgatccaggttctataggtatacctctagactacctctgtttgctatgtcctggcctattgcccctttatccagtaatataccaacaggtatcgaagggtgctttgatccagggcctataaccgggggttaccccaaaagtctattttatcctccggtgtatctggggaatatccccccagagtcccccaggataacgttggggtggcctatccccaggggtatagcccctagcccctccctaggataccataaggctagtccgggggtcaaccccttgagataccgttcatcgggtggaccctagggtattcataagaaaaatcttagggtattcagggggtggatcggggggaat

SEQ ID NO: 4 - DNA, 45703 bp, synthetic construct
  misc_feature 1..45703: note = Nucleic Acid Sequence of ECACC 17062002 Pa223
  source 1..45703: mol_type = other DNA
ccccaggagtcccccagataaagtgggggtggcctatccacaggggtatagcccctagcccctccctaggataccatgggattagtccgggggtcaacccttagacccttgggctaagccccctagagctaccgttcatcgggtggacactagggtattcataagaaaaatcttagggtattcaggaggtggatcggggggaatatcccttgataaccccttgaccccctattgacttccctcgataccatatgttaatcaaagactgtctatatccgggggtttgctgaagactgatagtccatagactgtataaaaaaccagtagacaaccccgtaaggaatatggcctaatggctccgccttgaaggaaaccgacaggcaacgggggaaacccctaggggcttgacaaaccccgcggaacctggcctaatggccaccacgttccacctaggccaccatcctccaagtctacggacattaacgggggctgccaaccaaaggggaatgcccaagtcacccaagtataagaccggcaggcatcgggggttgacaagggataggggaagcggtaagatggctcccaagtcaacggcataacgtgcatgactccctcgggattagcgtagccttgacaagcaagactgatggccactcagaaaaagagcctatgccggagaaccggacgaactaacccccgtcagagggttgacaagccaagcccgaggctctaacatgggcacccattaggtgacggccaccttgggaaagtgtaccttcgacggtccttggactgaacaagggagaaacccgaaagctgggggaataacgcacactccggaagagcgtaggttctggccttgtaccatagaaggtccgactggttgcctgctccagcgtaaacaattagcagagttcccggtagaatcgtggggaacagtatgatgggttagtatcgtcggcttagcaacgagggcgaagcgtggtagatactggcgaaggtaggggcgagggattcgaagacccccgaggatagccagaggacagaccacaacgaggatactaacgcggaaaggggccggctacgccaatgccaaggtttgccgactaacccctttatcgtatcgtagtgtgtgctacggctgatgattccaaaaggatgaaacgatgaacgctgctctggataagaagctgcgccgcaaggctagccgcaaggctcgtgctcaaggtctgaacattgggcatcttggcaaagctcagcagcgtgcagaacagaagttcgggatcatttccaattgtaacaagatcctggatagtaagtttgcttccctacaggaaaaggccggggcacgcaagcgcaaggctctgatgaataccgattggcgtaaccgtgaggatactaaactgcgcaattggtacaagcctagcaagtgcaagaactcccccgtcacccacatcaaacccgaggtataagttatgagcgactacaagcgcatcaacggcatcatcaagaccatcgcccagcgtggtgcgtccctggataagctgatccaaaccaccggcctggatatcctcaagcatatcgaagagcacggcgaggtttcgctggcatgcaagctgttcaaggccatgccccaggggtcccgtcgcaacgccctggccgcgtggttcatcgaccacggcaagatcgaggccaatatcgacaaggaaaagtccaaggaattcccgttcgtcatcgccaaggataaggccactcgactggagcgtgcagccgaaaagccctggttcaagtacaagaaagagcgtgacgtggccgacgagttctccctcgatcaagccatcgccgccttcaaggccaaaatccaacgtgccatcgacaagggccagctccaggcagctgacgagcgtatcgccgtgatccaacgtctggaagtgaaggacgaagcgaaggcagcctaacggccagggccaggggtgtgtactatgacgcgccccttggttcctccctagagtacatggtaggtccgtgtattgtatggatgaactaaccgaggataaaaccgtgtggaccccttttggtagctttctggtagctgttacaaacgattacgtgcaaggacgttgtaactcccgttcgtataacagcgatgggtctgagcaggtagtgtaccgttgggctttcgtcgatggtaagttcgcctgcggtgtatatgtagatggccgtcccatgggcttctgtgggtggaccaaatgaagtccacctacgaagctgcccacgaacgtgccctcatggttaatcggctacagaagctaaccagaatgcttcgtgtccaccacgaccctaagtggaaacaggaacaacaggaacttatcaagaggttaaggcgatgattatctctatcgtggtgtcttgcgtaggcatcgggtacttcttcttccgtgactggaaggaggagatgggtatcaactgacccaactgatgaggccatggtgattcctggccgaaacccccaccggacctatggttgcaggatggggagtcttgggaaatcagcaaaggaaaccatcccgtgaaacgcaacgactatcgtaaggtcagccgcaacatgcaagccatcgaggctatcgaccgcaagattgccaaggtaactcgtgagttgaccaactccagcggcaatcaccttggtaaaaccctggagttgaacaaactccgtgccaagcgtgcatcgctggctaagaacagggcgcggtaatgtcatggaagtatattgcatgtgccgccatacgctcacgttccaatgggttgttgctggtagcgtgggtggttctcttcgtcctgtcctcgaccttggacagcatcccttttgcatcgtgaggtaatccgatgaagattaatgtggtagaaaagcccaagaaggttccggcgcataccctcaaggctacagatgttttccgtgttgagaacatccatgggatggtacttcgggatgaccacaaccactacctccccaatgacggcgatgtgtacttcgtttgcctggaggctggtatcatttgcagcatccatagcgactgcgaggtcatcccggtggacgctcaaatgaccgtgagggtctagtgtactatgacgcccgccctgtatctgctcctctggttggaatccttggtcagtatccgcaggttccagcctaaccacaaggcgtaccctggttggtacgtcacccatatccgtgtgtctatcttcggcaaacgtgtaggtatcgtctatgaaatccactaaaccgcaagtcttctggttggtaagcaaggatggtgagcgtcacgtaaaggtcatcaatttcggccagttcatcaccgaaaccctttacgaactcggcattccggtggaccacatcgttcatcgggttcagtaaggtgacgccatgaccaatacctctttcaccgtagcagtggcaaagccaaggtttgagttccacatcagcaagatggcggacggtgcctacgtccattcgggtgattccgggtctgtcgacttggtttacctcaagaaaggcgaactcgtgaccattattcacaaggatggtttcaacatcctcaagtgcaaggaaatcggtactcaccctgatctattcccggccaagtggttgtcggtgaatgcctgcatgtgggggccgtatgaagaataaacccatgataggccagataatggcccaggagcgccgcatgaagcgtcgggtagaaaagcgggggttcaacatgagcctccaggaaggcacccaggagccccgtggtgagcttggtttcaccttggctgccgtaggtatggagtcgagccgatcggcttaccttcggcacgccagggaggccatgatccaatctggtgagccttgccctcattgtatgccaatcttcggtcccaaagagggccgttgctgcaactgcgcgagggactggtgatgggccggtcgaaacacctcacgttcctcccgtcgaggatcactgtccttgacgatgatacggtctatgtgccttggcgataccagttggaatccctctacaacatggcttccgctggtggtgctactgaccccgaggcagtcaagggtgaattccttgattgggtacgagcggagtatgggcgatatgtgtccatcgagcttgaaggaatacccgaggacagggaagatgccttcgatgagtatgtattgaaggggtctcagcaagggtaagcaaaatgatgacatacctcctgataatcccagcgatcctgatctacatggccttctccttgctcgtggctggcatcgccgggctggcggcaaactgcgatgagtacgggaggatgagccagaaggaccaggacgtttcaatcatcctcggtatcctgtggccagtgtcactgccctggatgtgcttcatgtgtgttatctggaaccctttggctaccaccatccgtgcggccaaacgactaatcaaaggagattactaatgcgccgtatagacttcccttcctgctgcaccgccaagatttacattggcatgggcccctcgggtaccgctgaccaccgagccggggaagcgtccaatgggttcagcccccgtggtttcgccaaggaattgatcggggtcatccgtcgcgagtacaacgaaggtcatggcaccgccgtgttctccgtgaacaacgagcaggtggtagccgataccatcctccgcagaatgggcagccactacaacccctgggcctccagctacaaccactcgacgaaggttcgggttcacgtcatcaacgtgcaagctgcggcagaaatcctcatcaaccacggtgtcctcatccgacgactcggtggtttgcaggactaccccggtactgtcgagcactcggaatacctcgacaagctctgcaaaggtctgtaacatctaggccttgacattctgctgaaagtgtggtataataaccttaaggtgtccggggttgctttatatccctaggataccttaaggttctaaccttctatcttcttcatcttaagaaagaggtaagaacaatggtcctcaagctctacactcgggtaatgctcctggctatccccgcatggataagcatcctctgggtagtaccacagatgctgtccccggctgacaccatggtaaacgccattggtgctgccctcctggtaatctccatcctgtactccctctgggctgtcagtcacctgtactcctacgcaagtaagctctctggaaagaaggaggtgcatcgtggccacggtaacgagaagcagtaaggtcccaaccagccccctttacttcgaacgtaagtgtgattcccagatttaccttcgttcggaatcccggaaggacctcaccatcccgatggtaaacatctgcaatgggagtgtcctgtacctacaccctcgggaactccccgtgtatttcgtggcgatacccgagggtgaaactctcaaggtgaaggtgtgatgtactatgacggaacagcagcgagaccgcctccgtaaggcaatccacatggatctagaaggtatcggcgaagatacccccgacctctacgacaacatacataagatagaaggcctgcttggccacccaggtaagcccccgcatgattggggcttctatgtactgatgttcgtcatgttcatttccttgtgcatggtctacatcactccccttctctacaagtaaggaatccctatgaagaaaatcctcggcctcctggccattgctgccatcgctatcaccgcaggttgctccaaggttcccgctggctataccggtgtcgtcgtcaacatgatgggcgactctaagggcgtcgatctgaaggaaaccccggtcggctggaagttcctgacccccaacgaggagttgttcaagttccccaccttcaaccagaacttcgatctggcggtggttactgcccaggacaaggacggcctgaagttggacttccccatcggcgtcaccctccgggctgctcctggtagcgcacccctgctgttccagacctaccgcaagggtatgaacgagatcgtaagcgtgaacgtgccgcaggtggtccgtaacgccgtgaacaacgccagcagcaagaagtccgccgaggccatctatggtcctggcaaggaagcctttgtcaaggaagtcgaagctgccgtccgtgagcacttctccacccgtggcataatcgtcgaaagcctgtacctcaacggtatgatcggtctgccggcgcaggtggtcgaggcgatcaatgccaagatcaaggccactcagacggcgatgcagcgtgagaacgaactgcgtcagacccaggccgaggcagccaaggctatcgcggcagccgagggtgaaaagcaagcggctatcctggtggcacaaggtaaggccgaaagcctccgcatccagggtgaagccctccgccagaaccccggcgtggtagaactgaatgccatcgagaagtgggacggcaagctgcctacctacgtcaccggcggccaagccatgcccttcatcaacgttcgctaaggaggtggacaatgggtaagcgtaaggttttctatgagcgtatctgctccaacggtgaattgaccctgaacggcaacctcaaggccaccaaggtgtcggcacccaccaaaggcaagcagcagcgtcgcggccccttctaagagggcgttagtaatgcccctaggaactccctgggggtattgctggctaactcaatcaaggagaaaacaaatggcccgtatcaagtacgccttcggtatgaaatccaagaagggcggggttaaggccctcaaggtaatgacctctgcctcctgcttcggagccatggagggggtggtaacccatggttataagctcgacggttggaccttcgtctgctcccgtcgaagcaagaagttcatcgatgcacttaataagtgtacccaaggtgaactcaagaccatcaccatcggcggcaaggcctacaagatcccgaaggttcacttctggtcttacgaaagcaaggccaagacttccccgttcgtgaagtacgttagcgggtcggagttgatctctcccactcaacacgggaaggaaggggtttgcggtatctacttcaacccgaaggaacataccctagattcctggtatcccatcatgaagttcctcttcaagctggtatccagtggtctagaccctcaaggtcgagaggagaagattcacatggagcttgctgaaaagtatggcttctggaagtcctacctcgcaatgtctttccacggattggtagccaatggatacaccgggtaccccctcaccaattacatgttcagtaatgattgggaggatatcaagaaaggcgatatccacatccagtccgcagaagagtgcaaccgattcggccgttgggtgcccaaccgtaatgccccaggtggacgtgagtgggtaaggtccaagccctacaagtcagaatacctggggattaagctgaacaaggtggaggtggtaactatttacccccagccaaccatcttcggcaagaaaaccccctacgacatcgtggatacggaactgggaggattcctccggcttactccttctgactgtgaaaaacacggagtggtcatgctggggaccatgggtgaaattcactggggtcgcggctcaagggatatccccctggccctggaaggattcatagagaaaaacttctgacaaccaaggagaaatcaaatgggaatgtatgcagcccataacgtgtactacgatgccgagggtgctgaggttggtcactgctgcgttaaatacgtggctgaatacaccaactgcttcggtgcattctgcgactcaacggggccccacccggatatagtgtgggagaccggggttggacacctcctggtgtctacccacgagaacactgctggtaaactggtagaattcttgaatagtgatctggtcaacaggatcaccgacggtggaatcctctcggcttcccaggactggcccaccgagtggtggacgggatctggccgtaaacaaaacctctccaatcctgaccacttcagcccacctggccgccgacaggctgtgtacgttcgggtggacctcaagaagaacgcatcggcaatcatctgcgcactccgtatgggtgatcgtctctggggtatcggggatcagatgcgtcgtattccgaaggaagctcaggataaaatcctgggattcaacgcagaacttcttaccctcgcggcttgtgcccaggtcggtagcactgcccactgcgacagctatccggcgatgttccctgtgactgctggggagtataaggagcagtgtgaagccaatggggatgatgtagaggatagctacatcagcgaaatcatggaccacttgacgggtgggttgaaggatatcacctacttcgatatcaagagcatccatggacgaactcgcgaagagttcaaggacaagctcaagcaccacaatgacccactctggctgggttataccaaggatgattacctgatcgagtgtgatgggttggatggggtacacgatgatcgaatcctttccatcatggcgcccatcaccatcgacgtaggggaccccggacgggataattcccccacggtacccaccggttattacgaccttcccgaaatcctcgaaaaactggagagcatgcaatgaacaacgcaatccccctgatcggtgcagatcccgaagttttcgtcggctacgaccgtaacccccagagcgtcatcggcttcatcggtggaaccaaggaagcgcccttggctgtagccggtggtgccgtccaagaagataacgttcttctggagtacaacatcgacccggccagcaccaaggaagatttcgtcaagcggatcatttccgtccgactccttggtgcccagatgctccaccccttcggcatgaacatcatcgagaacctgtcgtctcacctgtacgacgaggaactcctccgcagcttcggcccccaggcttatgtcttcggttgcgagccggactacaactgctggacccgtcgccagaacgtgatgccgaacgatgcacctccgaccctgcgcactgctggcggccacgtccatatcggcttcggccacatcgagcgagtcaccaaggctaccacccgcgaggtcatgcagatgtgtgactacctcctgggcctggcctctgtccttctcgacggcgacacccagcgtaagaagctgtacggtaaggccggtgcgatgcgctacaagccctacggcggcgagtaccgtagcctgtccaacttctggatcttctccgtcaacctgaccgagtgggtctatgaaatggcggtacaagcctacacctcgaagcacctgctggaagagtacaagtccatcgtgtctggcgatgaagtccagcggatcatcaacgagaacgacggtgttgcggcggtcgctgctcttcaggccttgggggtgaagtatgaatgacctcaacaacaggcatcgtctggccggcgacttcaacatgtactacagttccaccttcgccttcttccgggtggacggggagcctcgggtagtctacgtggatgacactgaatccatcggggatgaccgccagttcgatggcttccgtctcctgggtaatatcttccgtcccgacggcggacaatactacggcggcgtagtgtacagcgaggtcgaaggtgtgcggcccccgagtgggtactatgacgtcttcggtggcggcgagcgagatacctacgtgtcgttcctcgtgaacaatcgtacccagcgcaagggtgttgacccccggaatgtcctcatcaaccacggccagcaggcagtaaccggtcagatgatgatccgtatcttcctccaagcggaagagatgatctccgacccggcacaccgtgacttcttcatcaaggacggggtagtcaactggaaaggtgtaaaggtagggcagatggtagacaaccggctgtccgtagatgaacaattcaagaaccaggaggacttgctatgtcggttattggcacgcagataggtttccgtaagaaccagatcaagaccccggagcaccacgaggaacttcctgcggttgcatccttcgggttcgaggttgaactggagggtctggaccagtggcccgaggttgatggctgggatatcaagaacgatggatctctccggaatggtatggagtacgtcttctctggtccggcttctggtgaacaggcgatcaatcgtgtggaagccttcgcgaatgccatggaacagaatcccccggcacccaccttccgctgctctacccacctgcacatggatatgcgggatgtgaactggaaggtttacgagcggactgtcctggcctacatggtattcgaggatgtattcttcgatcactgccaaccgtaccgccgggatagcaacttctgtatccccttcttcagcaacgactggttgtcccagaccttcggtcgccgcatcctggcacccgagggtgatcgagagaaggtcttgggccttacctcctggccgaaatactcggccttgaacctccaggtcacccacaacttcggctccatcgagttccgtggtgcccacgcgatgaccactcgctcggaaatggtcgggctgatgcagcggatgatgtgcctcaaggcgttcgcgattgcccacgaggaaacccccttgggtgagttcgttaaggtcctgtcggatgtgaaccttcgtgatatctttttcctcggcgtagcccctgactatgaaatgtctccgggtggccgggaaatgggtatcgcaagcgcatccctggccattgcaactatgggctttgttcgctccggggtagaccccctggaggatgaacagaaccgtcagcgtcgtctccgtgagcaggctcgtgaacaacagcgggcactggatcgtcggttgatggctgcccgaagcgtcctgggtagaatccgagagggtgcccctgagcgttacaacctggcgatcatcgaagggagtaacatgcggatggatcaggtgcttaccactgtccagtccctgaggatgatcgggcaccaagtaagcgtccgagacctcgtagaggataacgagattcttcgtgattccttcgtacttctgctggacaacccggaccatatgcagcgtcaagctggattccgactcgaagaaaacatgtactaaggagataaacaatgtgtggattggtaggcttctgtgccacaactaacgcgagtgataacgaaatcgctcttctcaaatccctcctggccgtggatattatccgtggtgcccacgccactggtctggccaagatcgacccggttaagaacgaggtaggaattcacaagcgggcagtagatgcctacgacttcctggctgatcctgaaaccaaggagttcctggacaagggtcgggctcgcatctacatgggacataaccgttacgccaccatgggtgacaagacggaccatgcgaatgcccaccccttccaggtcgaccacatcaccatggtacataacgggactgtggattcctggggtctccatctgctggacggcaatgacaagtacaacgtggactccaacatgctgtgcgctaccattgccaaccacggagccaagaagaccttcgaagagaagttctccggtgctgctgcggtaatctggtgggactccaaggaacgtaccctgaacttcatccgcaacgatgagcgtccgttgttcatggcggtgaccaccactggtaccatcgtgtgggcatccgagcctggtatgctcaaggttttcctggagcgtcccaacgctaagatccgccttcgttctcccatcgctgaactgaaggctgaagtcctggtaactatcccgttcacggaggccggagtgcgaaagggtgtagaaccccagaccactccggttacatttctcgaccttcccatccccgaaagcgaaaggcaagcagcggcatggtggagtcgctacgtcggtgtctccgactacgaggactatagccgaagccaaggcagccaagcgggaacgaaaggcagcaaagcgggctcgtcgtatggaacgtctggcgatgcgtacgcaaggaacaccctccggatcaacaacaacctcgatgcagcaggcagcaccttcaagcaccgccaactcgtcaccttcgatgttgtcaaagtcgaggcctacgcaaacggcagcgaatacggaactgtcactggaatcgagcgtgaagaaaaccttctcatcgaagctcatggcatcaatgtcgccaaagtccacggctacaccgtcctccgaggaagtatctccaacgcctacttcatcggccaagaccgtgatctcaaggttactgtcgaggatctggcggtaagctgcctggacccaaagcatcggccgactcctggggagactaccccggttttgaggattggaacgatctcatcggagacgaaatcccattctaaacccagggttcaagtcggcggcaccttggggaatacccctccggccaacatcagctaccccttgaaggttcagggacacacgttcaacaacgttcatgtctttcgggacttcgtatcccaggggtgtgcatcctgcggtaagatccctaccgcgtatgaccagcgtaatcgtcatctgacggtgtacgagggtgccaagttcacaggtagcctggatgagtgtgagttcatctgcggcgaatgcgtaatcgaaaataaataggaggtcaaaatgacccaagtaactctcaagcgtcaagtagtgatccagatggaaaccgacgcaactcgtaagtatcctttctcccgcgacaccctggacaagatccagtcgatccgtcgagtcaaggagcaggaactcaatgatgccaacccggacgaggaattcctggtaccggccccggtagtcattgccgaggccatcgatcgactcttcgaagattacttcgagtaaagtagtgcgttagtaatagcccctggccgacccatgccggttcctaagctgcgtacatggggttcaacttaggatcacaggggctattgctggcttcactactaccctcaacagaaacaggagatatgccatgttctacatctacaaaggttcccgagcgtcttccggtgccgtaaatcttcgcaatgccctgggtgcccgaatccttcggtccgaggggtctacctaccgtggccgtgcgggtactgcggtaatcaactggggtaccgtaggtgcagaggcacgacgcctccagggtatcgccccggtattcctaaatgatccggccatggttgctcgctgcaccaacaagctggacttcttccgccacttcgaggccaatgcccctcacttgattccccgttggaccgagcgctgggaagatgccttgggtgtactaaatatctctggtcgcatgtatgcacgtaccgatctgaacggccatagcggtcgtggtatccatctggtgtgtactgtcaatgatgcagaagttgctgcaatcgatgccctccgtagacagggacactacccggtacacatctacggtcacacccacatcccggatgttatcaccagggcacaactgttcacccagggaatcgtcggtaagcgtaccgagtttcgagcccatgtgatccgtggggaggtagccctgctccaggtcaagctccgccgtactgccaacgatacggtgaccaacgaaggccaaagcatcgtccgtaacgtagccggtggctgggtctatggcgtcaacgatgcaatgggacgggatggcgctgagcaggctatgtcggcagcagcagaagctatccaagttgcaggcctggacttcggagctgtggatattatctaccagcacgctactagccgggcgtttgtcctggaaatcaacaccgcgccgggcctggatgcagaaggcagcgccctggaggcctacgtcaagggcttcaacaaaatcttcgaggagactatctaatggctgttcgtgttttcgtttatggtactctcctgtctggtttgtacaaccactaccttctggaaggggccgagtttgtcggcaatgctgtatcctgcgagcggggtctgatgtactccgctggcggattccccatcctctcctttgcctcccgcgctgacctcatcgtaggtgaaatctggcaactccccgagggcgaagcgggggatgaaatgctggagaacctggatgccctggagggttatccgggttggtatgatcgtaccctcaaggatttccgaatcaatggggaacgaatcaaagccctggtgtaccatcaggatagccatatggcgatggatatcgtcaaggatggcgactggaaggcacacctggcaaaacgacaaggagcagtataatgaacgaaatgaccgtagacaaagcagtagaagtctaccgcgataccccgaacaccttcggacaccaagagctacatgcccagaagatgctcctcaaggaaatcctgggacttgtagcttcccagcgacacctccaagactctatcgaggtctccaagattccggaggcctcggatagtcccgagaccagctacggtgggtactgtgacgaatccattggaattcgcttcatgtgggagcgactgaagaaaatcgaggatcgtcttcgggaactggaggaggtctacggtaccttcgtaacaactccctataaaaccctaccgggcaacgtgaatgctgtaccaagcctggttctcaagagtcaactggaggggtaagtgaagaaaatcatcggtgatacggcttgtccgggttgccgagctaaaggtggggataaaacaggaaatcacctcatcttgttcgttgatacagaaaagggtactcggttcggaagttgtaaccgttgtggtcactacgaagtcctcgaagagggtttcaaggttcctgaacgaagggagaaatccgaggaggatatcatccatgaagtcaacgaagtccttgagtatccaattaaagccctcgatactcgaaagatcagcaaatcaatcgctgaacggtacggggtacgtgttggtctatcacaagaaaatggtgaggacgttatcgagcattactatccacgcacccgcgatggggagtaccgagcgtttaacgtccgaatcctagaacctaaggctttctactatcgtggtagccccaagggtggtgttgaccccttcggttataacacccttcggcataaggatatgggccacctgcggttagtcatctgcgaagatgaactgtcggctatgtctgtggcccagatcatggagtcgaaactcccggagaagtggaagcatcttcgtcaggcatccattagttggtcctcgggtgttggttctgctgggcgggatatcgcattccttaaggagtctggtgtccttgagcggttcaacgaggtcatctattgccacgatgctgatgacgaggggcgtaagtcagtagaaaaggtacgtgccctgtaccctgagtgtaagtttgtcgagctcccgctgaaggatgctaacgacatgctcatgcgtaatcggggggatgaggtataccagatgatccgattcggcagcaaggtgaagtctcctgactgctccgtgactgtcgatgaggtatatgctgaggctctggaaccccctaagtggggcaagagttatccgtgggagggcctaaccaaccttacctatggtcagcgtgatggtgaaatcatcggggtaggtggaggtactggtatcggtaagaccctgttggcccacgagattgctgcctggaattgcattgagcacggggagaacgtagggactttcctgttggaagagcaggtagccatgacacttaagaatatcgcggggaaggttgccaacgtgcccttccaccgaccggatatcgagtgggatgagcaagcctttaaagatgctgcgggtaaactccggggcaagctcttcatgtggaagaataagggtcagaacgattgggatcatatcaaggagtgtattcgcttctgggcagtagccatggatgtgaagactatcctactggataacatgaccgccatgaccaaccaccttagtccttccgaaatgaacacggagatcgctcgtatctgtacagagctcgcagggatggccgacgagctaggactgcggatcttcatcttctcccaccttaacccacccaagggtaaccgtacccacgaggagggtgctgaagtaaaggaaagccagttcactggttcccgagctatgcagcggtggtgtcagcttatgatcggattcgagcggaacaagcaggctgacggtgaagagaagcacgagagtcgaatccgtgtaatcaaggacaggaactacggtaacactggcctagtgttcaccaagtataaccctgagacgggtcgcttggttgagcgcgagggcagttacgacgaggtacctgctgacgatgacaccccaatttgattacgtgatctatgaccttgagggggacggactcttcaatgaggtcacaaggctatggtgcgctgttgcagtagacattccgactggggtagtccggggattccggcccgaggaaatggacgtgttctaccgcatcatcgcccatgcaaagttcgtggtcgggcataacatcctagactacgacaaccgggtccttgagaaacttcatgggatcatcatccctccggatcgaagctacgacaccttggttgcatcgaggttgacttggccagaccgaccccagggtcattccctgggtgcctggggtagattcctgaagtgtcacaagggtgatttcaacgacttctccaagttctccgaagaaatgtttgagtattgccttcaggatggagtggtcagtcacgcactgttcaactacctcctccgggtactcggcatgacttggcaagagcttgttgaatggaggactgtagattggctaaaaagcgagtgaggaactacaagcgtgaaagggagctggctattcgacgcggcgaaacgggcgttggctctaagtctggagatgctcagcggcaccgagcccgccgaaaggtggaaaagcgtcttggaaggaaactcggagccgacgaggttgtcgatcatatcaaacgtgttaaagatggtggcggtaacggggattctaatctccgcgtccgtagtcattcttctaacgctgctgatggtggtcgtgtgggcaatcgtaaggccaaaggcattcgtaagaaaaagtaactaaggaggggccttcgggcccccgaggactctctatgttcaatcgaaagcttagcatcagtaacatcctcaattccttcgataaggttctggtgaacctgaagaccttcatccaagagtcttcggaggaatctgagcgcatctacaacgagatcagcctgctcaaagctgagcgtacccaggtcatgcaggacaacctgaaggcccagaaggtactggctaacctggaagaattgctgggaggtaagagtgaagaagtatcgagttaacgttgggttccaggacaccaaggtgttcaacgcagacttctaccgcatcgagttggatatcatccggttcttcgcgggtgattccgatgccaaccccatgaccgtccgagccaatgagatcggtgctgtccgtggatgggtttctgtggaggagattaacgatggcgagtaagaaggaaagcctggaggatcaggcacggaaggagattgccctggagaaggagttctctggtagctggggtggccccgagatcgatgctgatgacttccccttgggtagtgcctgtggcctagatcccgaggtatgcgagtcatgccaatagctctacaagtccttgtcatcttctggagtgcattcttccaggtattcctcctgggattgaactctaagctccttcgggatgacaagatcaaggctgggtttgtagtgtcttggtgtatcacgctggctcagtttgcttacatcaaggccgtggggtcttcccacttggatatcggatggtttatcttcgtatccgggtggggaggtgcccttgggattacctctgcccaatacttctacaagtggtacgacagagtattccacaagaaggcttgacaaatccagaaaagtgtggtataataaccttaaggtgtccggggttgctttacccctataggagatacaaatgagtgaccatgtaagctattccaaacatgtccgtggtaagtacctgtgtaatatggcctctgccctccataagagcatggaggtacaaaggactaacatccgtaagttcctcagcagtccccacattaccctacgggagaagcgtcgggtgttcctgagcctgccagagggattcctgggggtgagctatttcacggggtctcacctgaacctgagttcctactcggatcgtcgaaacacccggattcgtgacaaggatatgagtctctatgatgacttctatgtggacaggggcacccaactagatcctcgggatgctttgtttgccaataaagaggcgaaggagtacgagtttcagtttctgaggaagaagtgggggactgacttcgagttgtctggtgaggatcttgagaacgtcaagggttgccagcgcatgatcgatctgtcctggtatctggtagacctggcctgtgaacgtgggtgttcctacttcgttttcgactggtgattacatgagcaagatcaagagtatcctcatggagcgggtagatgacttcctgctcaagcaagttgctgtggccttcctggagcagcaatggaagctggaccgcagtggtaccgtggactacctctcctacctggggggtgtatctcacgaagctgtggagacagttgtagagaatctagcggagcgtcttaagggggattaagtggattggagaaagtcactgttcgttgagcacaaggtagctgatatcatcagccgccagagtaaacgtggagtctacttccagactcagcgggccaagtggctgatccatgtgcttagcgaacgaatcctcaagattgaccttgaggctgtcccccagatgcccatgatgatcgtaagggctggggccttcagcaagccattcctaaagagtggtaagccgaaccaaaggctccagtccctgtggcaacgtcttgggcatttcgaggtatctggacccttctctgcaatcgagtatgtacccttcgaccttggtaagactgccaagttcaaggactggatgctggatcaggggtggatacctgaccaatggaacatcaaggatattactgtcggcactgatggcaagaagctacgtggatctgaccttaatgaatccttgaataagtacattgaggatctccgacagagcaagtctggacgactccgaatgaagctccaggggatcatccctgggaaaactaccattggggaagtcaagagaaagctcgaaaagcaacgtaaggtcctaacgactccgaagatgactgaagagtcgatggataccgtccagggagacctgggcaagctggtgatgcagcgaatggtttgggcccaccgtcggtccctcttgcaggggctggtagatcaggtgaggcctgatggacgcctagaggggagtgctaacccctgtgcaacacctacgggccgtatgaggcaccgtgtagtagtcaatatccccgctgctcgttctcctttcggacctgaaatccgtgggttgttccaggggacacctgaggctggtgaatggaaatggactgtcctccgccgtgatcttggggagaacgaaagggttaggcccttcactaacatcgtggaggaactcaagaagggtaagtggaagactgtaggaaagtaccgggtatacgtcccagcgaaccaattagtattcgtgggctacgatggtgctggtctagagcttcggatgcttgcatcctacatcaacaacccagagtacaccaaagaggtggtcgagggtgatgtacacacggccaaccagatagccgcagggcttcccacacgggacgatgctaagacgttcatctacgccttcatctacggtgctggggatgccaagattgggactatcattggtggtacaagggcggacggggctaagctccgggcccaattccttgaagctaacccagaccttgctgcactgattgagagggttaagcaggaagccgagaggggttatctcgaagggctagacggacggaagctaaccatgcgacgttctgagtctggcgacgtgatgatccacaaggcattgaacaccctcctacaagcagcaggtgcaattgtcatgaagtgggccatggtgctcctagacgagcgagtccgtaggttaaacctgagggcttggaaagtcttggatatccacgacgaaggtcagtgggaatgccacccagaggatctcaaggcgctacgtgagcagatggaagtctgtgtccgggatgctggggagatcctaggtgttaactgtcctttggctagcgattctatcgctggtcgctcatggaaagacacacattgatacatctgggggttgactttcagcccccttttgtggtataataccttcttccctacgagaggtttaagatatgtctaagaaagtttcccaacgattcaccttcccggtagcgaagctgatcttcccctacatcgtaactccggataccgagtacggtgaggtctaccaagtaaccatctgcattccgaccaaggaagaggccgacaagctggtacagcagatggaatctaaggatgttcgtctgaaggacaccatcaaataccaagagcgagacggcgagtacctgttcaaggttaagcagaagaagcacgtggactggatgcaggatggcgaacgtaagtctgccgtgatgaagcctgtagtcctgacctcggataacaagccgtatgacggccccaacccgtggggcggttcgactggcgaggtaggtatcctgatcgaaacccagaagggtccgcgaggcaagggtactatgacggccttgcgactgcgcggtgtacgtctccacgagattgtatccggtggtgatggcgaggatgatcctctgttcggtggtggttttgctgaagaagaggacaagtccgaggacgtattcgacgaggtattcgatgacgaagacgctcctatctaagggtctagcggatcatgagtcgggggtatgccggtcaggctgcccctactgcttaattgaattcgagaaagtgtggggtgtacgggtggtgagttctacagctgcatctaataataaagtagaggtcgatcctaatggaatcaaacctggtgagccgggcgctaagctcgatagcggcaaggtggatgttggaatcatcttcgaagcgttcccgagggctctatatgcagtggcacaagttgctaacttcggagccagcaaatatagtcgcgggggttggaggcatgtcgagaacggtatccagcgatatgacgctgccttcggaagacacctccttgagcgtcacaagggaaaaactttggacccccaaagtaaactaccccaccggtatcacgaagtgtggaacgccctagcgtccctggaactcgtcattcagcaagaggaggaatccaatggaacttctgttggacccaagggctaagactgttcctagcaactactctgtgatgggaatcgatgtagacctggggcttcccccaggctacagcctaacggaggaagctatggacaaagccaagcgtcaagagagcgagtattacgactggaaggggtatgaagcactggccaatccagtgatagagcacccagagtaccgagccaagggtgaagcctttgccctccgtgtcttctgggaagagaagctcaaagaatcccaggtcgtagaagaggtatcgtgatgattgaggagcggttctggtccaaggttgataaaaccgagggttgctggaactggacactaggtttcccatacaatgctgttcagaacatcagatatggcagggcgtgggctcaatgctgggagtagtccagcgtaaggagggtaagtagtatgatcgcaggaattgatggcgacgtactttagcttcgctacgaattgggacatgtggctatgtcgaaggaacacatcttcgatatccaggtggagaagccatggcctgaggaagaggtccataagctcgtcgatgataaagtcgaacaaattatcaaaagggtaaatgcagatgagtgtgaaatctaccttactggccaaggaaattttaggctggagcttgcgaaaatcaagcaatataagggtactcgaatcggtcttgaaaagcctcatcactgggaaaccgtgtcggcccgacttaaggacaagtggggagcaatcactttccacggtatcgaggctgatgactggctcggaattcgagggactgaagagggagataactttacagcgtgttctagagacaaggatatccgccaagtcccaggatgctaccattacagttggccctgtggagattcccagccggagttgggaccatttcaagttgatggtcttggaacagtctccgcttcttggcgaatgtatggtgttaagaaaccgcagaagtcatggaagcttgagggcaacggtacggcattcctctacgggcaactccttgttggtgactccgtggataacataccaggcctcccagggacgggaccaaagacagcggcagatttgcttggggagctttctaatgagagggatctcttcgcagcttgcgcttacgcctaccaacagaagtacggagataattggaaggagtaccttgtggaaaactttcgtctcctctacctcattcgcgaccgctcttggcttgatattcagcagtccggtaacgagtatcactgctcactgaagaaacattgggagatcccctatgacgacgaagatttattctattgatgaggccctatctatctgtgagggtctcttcaaaatcctggagactactggcttcactgactacaaggtggctggtggtttcctccgggatgcagacaacggggtagcacccaaggatattgacctatatgtacgcaggcctcgtgtagaagccattggtagacgagtctcacgtgtctttgggccccagatggttccttgcagtgatgatgagcttgaggtagaagtcatccggttctacaacaaacttggtcacaagaaggttcgtacggggtccggggaattacgcaatgggtatcccgaggggttttctgtgtgggagtccattggtgttgatctcccggttaacctggtcgtatccactgacagtcacccagcagagttcgacatggggatctgtgaaatctcctgttggcctattggcacccttcgtcgtaaccgtagtggagaaatctaccggtccagggcatacgagtgggacaaggagaataagtgcatcacaatcaatcgtatggttgatccgttgctggagagtggcaatgagataaccgttggccagatcgagagactcgttaatcacgtatcgagggtaaaggctaagtacccagaccaccgagtgtgcattggggattggatttggatgcttcttcgttcacgttcagtctacgcagggagcgcaatggattttatagtaaggcttcaagaggagggtctaattggcaaagcaggggagattcttcagaaccaaactgaagtcattgattgggacgaagtacgacagcgaaaccgagaagatcgtccacgagacgatgccttggatgcagttcaagccgtccccggtaccatacgtcatcgagcacaagtacaagccggacttcaaggtatcgacattacaaccttgtggatcgacgaagaatctgttggtcgaggtcaaggggtacttccaagaggcgtctgaggcatccaagtatatctgggtgagggaggcccttcccccggatactgaacttgtgttcatctttgagcggcctaatacggcctgccattggcttagtaagcgtaaggatggcacaaagcaatccatggcggaatgggccgaacgtaacggcttccgctggtttactctagagactttcaaggagtccttccctaatgagtaagaagtacaatgaagacactctcgttatcgcggacactcaagttcgatccgaggttaacatcgaccacattggtaacctcggggagtggatcgcacgtaaccgccccaagcgaattgttcatattggggaccattgggacatgcccagcctgtctagctacgaccgtggtaccgctaagatcgaaggccgacgagtcctcgctgatatccaagctggtaatgatgcgatgcgagtcctgctcgatcctcttcgtcgcctacagcagcatcaggcgagttgtaagaaaagggtctaccgaccagaaatgcacttcttcatcggtaaccacgaggagcgtatcaagcggtatgaaaactctaaccctgctctccaaggttttattgggtacgatcattttgatctgtccgattggattgtccatgatttcctcgacgtgggtgttatcgaaggtgtcgccttcgcccactacttctacaatcccaacagtggtcggccatacggcgggagtgccgagcatcgcctcaataagatcaagcgcagcttcgtccaaggccatgagcagggattcaagtaccacatcgaggcagtaggaaagaagcgaatccacggattggtagtcggtagcttctatacccacgatgaatcctacaagggtcctcagggtaacgaccattggcgaggtgtagcccttctccggaaccacaaggacggggaatatgacctcaagctgatgagcgtggaggagttcctgtgagtaagttcttgccagacctgtactacattaagtctgagcatgacttcggtcaacgggggttggcgtttaagacgccgatctccgcagaactctggctggacatgaagtttgggaaagggggtgcagaggaggggcttaagcgagggatgtattccatcgaagtcctggagatcctctacatcccaagcgttcaccttccagatatcttggggtaatctatgaaagaccgagtgggacgtaagctagaggtaggggacagtgtagtcttcctggtccacaggaacacctcctcccatctagctattggtacagttgatgggtttactccgaagatgattcggatcaaatgtcctaccatgagttggactattgattctgaatatgtcctcaggagcagtgacaaggtggtgtactatgacaaaggctgaactggagaaagcacttgaagagacgcaaagcgctcttgcgaaggctgaggcgaaggccttttcctttgaagaactggctgaagaagctaaaagacagattgaattcctcgaagggatgctagacctagtagacctcagggcttctgtattctacggagattggaggggttatgcagaaagatcaaaagggtaagggtggattcccttggacctacattgcggtagcagccttgtttgccctgctggtttatgtagggtatagctgactgatgttactcctgaccctgggagaaatatctaggttactaatcgaagtgttatcttgggcaggttccttatgaagtatcgagtgcagcagctagggaagcgtctcttctacatacaaaagaggtttctctttatgtggaggactgttacggactcgggaggttgttttatggccttctactccttggaagatgctgaggagttcatccgggataacctaaagtatagggattctccagtctaccaccccgtcgagtaaccaaaaaaaaggccccaagggtatcatcccaaggggccttatctttagctccggagagcgttcagcagagtgttgaacttatccacgagggcctcgtgagtgtccgtataggaagccacggggatctcagatatcttatcctggatggaagccgcaatggcttcatataggccactcacttcgctagccttaggcttccagttacctggtttagcctcactagccttagcacccacctccagaagcttaggcttcccctcaatatcctcccacttcactggctcaaagtctttcgggccaatgacttgtcggagggtcccatcctcattacgaacagcaatagccttcgattgaataacgaatacactgttgccgtcgttgtcagcgatataacctgccataattaactcctaattaagctgctgatttgaaggtacccacggtaccaccgaagggatatacacggatagtgcattccccgaattgaccatcagtctccgtctggttacccttagcataccaagccttgtccggagatacagcaaacttgatcttagctgccgatccctggataaagtggaagtgacaacctggggttacatcggggcccagggtgacggtaatctccgtagtcccctgagacatgaagaaccatccagactgttgcttagtcagggtaatattctgagtaagctgctgggtattgacgacgttaccacccaggtgagtagtcacaaggcggggatcaaggtagttctgttggagagaaccaatgttctgcacaggaccacgcatgtcgtcaaactgctcgataatgtagtcaccaatggtaccaccattttggtcatctggaccaatcacgtgcttgatagccacataaacatccaggtgttcccgacgggggtctactcggaattcactaccgaaagcagtaccacaaggggcctcaatcatagtctcagagaacaggccaatcacgtggccagggtcacggttaccacccgaggtttcaccaggagcacccacaagggtgttgaccaggacagaggggataacggcctctgaggggttctccgggaaggaggctgtaacgttctctactaccagggctgcgttctccttaactgcataatagtcagggaggaactctgtagcggtaacgatgttagtatttacccgaccggcatccgaggtaatctcaatgagatcaccatcggcattacgtcggaagacttttggcacatcaaccacgtaggcacggccaccaggaccagcggcatcagtttctacataagttgccatattacttctccttaaagatccagagcatcttccatcgcttccataccggcaacgaaaggacgtgctggagtttgcttatacaagaacttccagatttcacctgcggaagggtcatccccgaacgaggcaatagttttaccaacagccatagcaccctcagttgcagcaccagctactggacccaatagtacctctgcgggggtagtccctcggcggtaacccgtaagcatgtcgtagatcatagaggcctggagtggcatctgttgcatcactacgtccatcatccgttgttctggactacgggtatcctctcggctagacccaccgaacttagccagttgacgaagctcatcctggagatacccaagactcatcatcagaccaagagtgaaggctacacctgcggcacccataccagcgttggtccaagaaccagcgaagtgtgggctcattcgtctacggaacatcggtaggatgatgttaccataggctgctggataacccttcaagagggagaacatctgaacgtttccattgctcatccacataggcttatcagcgaaggttgggtcgaggactacctgatccacaaatcgacgcatagccagagtcttgacgttgttagccatcaagacttcagatggtgtagctggggagaccaacttgagggcatcctgctggataccgatgttaacccccatttcccgaagctgagcaaccttcagagcaccattagcagaactgaaggggaggcctgcggctagatccatcaggttattctgatagacccgcttggcagtctctgttgcaaacacccggttaacatgggttagaatggacaagccgttgatgaggaactgaccacggattgccttttggacagtcgtgttaaatacctcagcaccaacccgatcggccatcaaagaggtagcagaggacagggtgtggttcatatcactcataaacctaccggtctcagattttggaaccccactgtagatcctacgggctgcctgtcttactacctcccccatagttggcaatacagccccaagggtaggcatcacccctgccttagcgaatggtaggctgaactcagttagggtagagaaccctgcgaggggtagtcgggagagcacgagggcacctgacgtaacagccgccagcttcttaaggttggggtctttgatacgaccgtgcataccattgtaggcatccactaggtcatacatccgatccacttcttccttagtgactctcttaccagcccgttgagcctcagctacagcagaagcaatcttagcgttagccttctccccattgataccaaacctctcggtaaaggcaatccggtgggaagccccctcgaagtaatctcgaatttcctggagacgcttcttaggagtatcgttgagagaatacttgttgaggatctcttgaggcacggagccaaaggcccgactctcttccagctgaccatacttaggtactgcatcactctgggcaaaccgtccacgtagagtatctggatcaccttggatacgatatcgagggtctacttcccaagccccggtctgctggttctgagtaaccagtcggttaacctcaggggcagtgttaccacgagtatcatccgagacttccgccagccagttggctacagcatcctcagcagcttgacggttctggaagtacggagtgatgtcgttcaggaactcgggggtctgaaccttctctggggatagaccgaatggcatatagttggggatagtaccaacggacattccaccacggttaacagcctcattccgtacgtcatccatcaaagcacggagacgggtagcttctggggtattgacacctgcggatgtatctgagataatcctgtcgatctctttcgaagacttaccctcgaagatgttatcgagttcagagttccacttacctgcctggagttcctgatcctcaaagatagtcttaccagaggctcgcttaccactcatgtctgccctgaaagtctcagagaactcgcgggcgataggggaggccttagcaagtggctctagaagggacgtagcttcgtttcctagggcatcccaggctttcttgaccgtacccctaggctcaaactcagaagccttaggaggggcctcaggggcgttaggatcaaccacagcagatccagcggagtcctgatgacgacccagggtgtccaaaccggaggatacagcaccaccagcagtaccaatagcggtaccagtgaaggcagccgtcaggaggttatccatgaactgctctggggtttgtacctgccctactgcatcataagcaatagtatcctggagagcctgttgagcaccagaggttacaccttcagccacaccagagactgctgcatgcttaccagcctgggtaacagcctcgatagcggtttgcttagggagtccagactgaaccagcatctggtatgccccgtccttaccgatgtgcttcaagagagggccagcgatgaccccagcaccagcggtatccagggcagacagtccagcacccccgataactgcggtccagggattgctttggtctgggtctagctccttcatctggttgctcagggcacccacgttgatacccatggagcttaggaaggaaccaatgagcgctccacccatacgacctggggccccaaatacagagccagccttagcccctgctgcaccaccagcaagtacaggggccatcgaagggagagcctctacaatgttattcttgaggaacgaaccgatggatgggatatcttggatatcagcgaaagaccgaacatcaggggttccgtactgtgacgcttcctgagcattctcctcggccatctgtgtgccgtagtctttcaggtagtcactgccagtcagttcaccaagggtagcaatggtaccaccgatgttagactgcatggtatcaaccccgcgaccaatcgcagagctaatagaattagggtcagccggagttactagggcactcaggtctggggcaggctcaggggtagcaggggctacttcctggggtgcctcttcgataacctctggctcattcaaggaagccaattcagcggctacgtcgaggcccttgacctcagccagttccgcatcaatagcggccttcagttctggagaaagagccataagtcaccttctagttgctggaaagagaataggctcttaagatgtatcttaggatactaaccttcttagtaagtaagtcaaaaggaagaaggaatcttaagagcctataggtctattatactacattttggatactttgtcaagtaccttttatcgggttggagcaggatagtccagatcaccaccaaagaaccagttacccgtaggttgaagcttagcagcctcccgctggataataccaatagggttagccccagggttagccctcatttcgttacgaagggtctgagctatagcctgttgggcagtcttaccaagcttcttaccacccagggcctgagatccactgacttcactgaggatgcccaaggcatctttggtagtaaccggctcacctcgggctgccttagctgctgcctgacgtgcctgagcgctaatcttagcagtcttcagacgtacagcagaactaagctgggcattctcttgagacatatcctggccacgacgggttgtttcagcctgtagctgagcccgttggttagccatatcctgtccacgctgggttagggcaaggttggctcgcccccgagcagcctcctcagcccaccgatccagaagaccagtgttcttacgagaggtttcctcaagggtgcccttcttgacctccaggttcccttccccaagacgtacatcagcagcatccttagctaccttacgttccagatccttttccttgatctgacgatcagcagctgcggcagccatcttctgctggttgatcccagcctgtaggttacgatccagttgacgctcataggaagcagcgaacatatccccggctttaccagtcttgtctatggccgaagccagaagaccagtaccaatgagagcataagatacataacgagacaggtcatcattatccatagtcctcatctgggtcaactcttcgttcacccgattcttaagctcttggggtttaagctctacaccctctctctgggcatcagcctcaactacagcctgggccatttcaggacgacttacagcaccagtacgaagaccctcagctgcaccctgttggataacctgacgattagcctcctcctcatctgctacagcagcaccagcctcggaagccacctctggggtgatctcaggctcgatagagggttggttaggggttaccccataggacaggagttcagccccagtagggcctcctgtggcgttctgggctgcacgttggcctacaccctgggcccattggtttgcctcttgagcaacctcaggccccatattctccacagcctgactagcctgagccatttcttcagcacgagtccgttctgcatccccgagttctcctggggttacagcagcttgtatacctacagatacaggaccaccgaggatacctgcgatacgacccagggcaccacgacccgttgcttcagccgcaggggctacctcttgagcagcccgagaccaacggtcagggttctcaagggcagcctgagcagcacgacggacacgatcagggagtccagagccttctacagccagcggcagtttgttccttgcgattcgcgccatatccagttcattctgagcagcccgaaggatgctagggcgaattggggacccttcataagccatgctggccataggattaacctccaaatagtttacttaggattccagacttgggttgctgtccgaaaccgagttcttctgcgctaggaagaatgcccattcccgctcccatacctgctgcaacttctgctgcgcctcgttcgcttgctcccctagccccttcgacagaaaagcttggtactgcttctgaagaaggctcacttccttgcccgaggagagccgcgagtgctgctccgcccattgcgccaaaagggtttgattgtccactagactctcccgattgcattgtcccaaggtgggtggggcggtaggaaccttgacggctcaactgtacccgctcctgcccaccacctatattctgtcctatagcgaacagcttctgaggaagggaggccattagaacagaaggcccccgaccctaccaccagtattcataccaacagaagcacccgctgggccaccgaagagagcgcccaaggaagcaccaccgagagcacccagggcagacccgaggccaccaccaccgccaccacccgaggaggtagtaacgttggttcctcccatgtcaccggagataagctccttgtaggcgagtaggtcgttaaggctgacgttattctcataggcccacttctgtagtgccccattgatctcctgctgctcctgattctgaagcatactaccagcatctacctgcatggcattaccagagccgaggcccttagcaatagccgacaggttacccagggtattcaacctattctggttgtaagcctgctggtcttggaaagccaactgggaggcgttattctgttgattctggagcaacctagcggtagcaataccttctgctacacccgctcgggaactcccatactgaccagcattagtggcccctgctcgcaggtctgggcgtaccgtagtgtcgaagtcccattgcatctgctcgttggctgcaccaatggcattcgccaagccagttttgttgggatcgtaaggaccaaggtaatcagccaaagagctaacacctgagctacccaggagagactgaagggcacccccgagaccacccaacccttcgatgccgccaagctggagagcattttggtcagccaccgggtcgaagttcggatcacccccatagttagggtcaaagcccccgttatgtagccaatcactggcacccgagagtagttcattatagttaccttgctgatagggtgtagagacagaggtggtcttttgcttcttactaccacccttgtaagcccgagaatctagggcatcctctacatcgaaccccatcagacgctttacgttaaattggaggaagttcatctggagttacctcacgatagaaggatacggagtcttcggtgtacccgagtttctctagggtaggcttccagccccgacgaccttcgcattggataaaccgacagttaactcgttgggcgaactgtccgaggaagtcgtctacctccgagtaatctaccggggtttcattcccaggcatcttaccactccagaagaagtgaaggatgttacccaggggtgcttgggacacttgaattacaccagcgtagccactctcttcctgatagaagacataggcttcgtagttaaccaaggagtggaccaagtgttcgaagtcccaaaacttaccgaggtccgtcctattgaaggctcgggccagagcagggactacggtaggaagcagatcgatattctcacgagtaatcaaatgaatcatggagtcaccaccagggggaatgtgaatgtacccccgaagactgctccttggggaattccttggattagaacacggccattagctgtgatcttaaacattgcttggttaaccaccccagtctgaatagtagccctgttgagaacatcaaagacttgatccaacggaggtgcgttagggtcagacggaggtggataagtaatagtgctctgcgctgggatgatgctggaataggccgggataaacaactcagcagggggccaatacgcctggggaagatccaggacagtagctccattagtgtagttaccaccagacatgagcatggttatccatacttcatccttagcagtgttcatcctataggcacaggtacccatcggttgatggttgttctgtggggtaaataggataaagtcccccggcaggtccttgggtttagtacctgcgagtctccattggttatccaagtcgtaatgatagatgccggatactggacctaccactccgggggcaaaatactttacagtccctggcttgagcttcttagggggctccatagagaccccccagtagccgtcagctaggtcattaagagtctgtccaaccctaacgaattcttcattaaggaagggcagcagttcctcctcttcctgtggtggaatcgaagggctgtacttttgactcatcgcatacctgccttcggggccatttcaatagtgtacccattgaagtaccaatccccctccgaagagaagtcgaacttcaaggctatataccggcccacatgtttagtgtcaatcttatagtcctgaccaatccggtatgggtaagggcctttccatcgaataccagaaccttgtacctgagcgttacccacccagatgttacaagtaccgttaccagtaatgtgtggaatgatggcactcactgtcttcatcattcgatcatccccgagatagatatcggatctctcaagggtactgacgaagttctgtccagagaatgtagagttattaccgaagaggaacaatttcttatcctggaaagatgagaagatcatactggactttgctgggttataagagccttcaccccagaccgaagtataggtatcccaggggttggggtcgtcatcccagaggttagacaccttaggatcgatgatcccgtaggctccactgagaacgttgggaaggtctcggatactccaagtgttttccttccagttccagatgatagccctatcgcagtgcttacctggctcagacctagtagaagagtagcatacccacatttcagtattcacgtggtctgcaattacgaaggtccgttgatagttgtcagggttaatatccgagaagaagaacttacggacctgagcatcaataacagactgcttctgcacaccgttgtggacatatacgtcaccgtggcctactacaaagtggttaccatcgaactctattgcacagttgggtccgaggatacctacgtcgttaaacagctgctggaattggaagatgaacaatccaccgatataccgcatggagtatacagagtcttccttgtagataatgaaggagtcacgaagcttaacaccatctacgatggcaccattggtatcagccaaggtgttctgaccagcatccttagtagggtccgttgggtcccaagatacaggaatacccccagcatcagccgaggtactccaccagaccatctgtggcatttctacagagttctgggttacgttcaaggcaatcataaagttcttgaacgacttcattcgtttagcgaacgtgttagccggccagttaggcatgggaatgaacccactattctgtggcatcaacacgtaaggatagttgcttgggttgttagcaaagatgacaccattgaatgaaccagaggaccatctactggttacacttgcagagtgtccacctggggatacatccacaacggtagtaccatccgccaagtacatacgctgttcgccacacaggagccaataggggatgttattccggatgaaagggaacatatccaagattggggcctgggctgtatcaaagataggcgtatggcccagagccttctgagccttgccgttcttaaaccggacgttgttcccgaaggaccatttctccagtggcaggtcagcgggggcgatatcggtcacaatccccgtagggttcttgacctcttgtctctctagggccattgtatacctcagttcttaatgatgaagaacacagaacagaacggtgggatattacccaacggcatgttgatctttactgcatggttgtgagtctgaccttgaccagcagggccagtctcaaggttggtattggctgcgttaccagagccacccgtcagagcacccgagtcacccgcagaaccggtgagggtagtagcccctcgggatctccaagtgtgggtatgtgctgggatttgggctagggtaagggcagtcccttcagtgaacccatcccatacaatgttagcgctaccgcctctagtacctacagcctgggaagatccgtcgataccccaggggaatgcaccaatgaggttggggacggggataccattagaggtagtccctacaccattacaaagcttccaacctgctgggatctgagccagtgacccagcccacatgataaccatcccaggtttaacatactgggttgtatcagcgactgcgtttagctgggctgccgttacagtgacagcctgagaaatattggggaaggtattcttaatagcactcttaatgagtcgcaggtggtcatccccaaaggacttcagatcagagccggtagggttcgtaggcaccaactggttaatataagttgcgacctcaagacccattcttggcctcctttatctcttctttcatttcttttcgggtgacataccgctccccaaagattgccatagaaatctggagatcgctcaccgctttggtgagcttctccgtggcctggatgtttctttcaagcagagcctgatgaacattctgaccgacaaccgaggaacctaccgtcactaccgaggatacgaccagggcactgacgatgctgcccaggttatcagttagaagttgcatcctccgtcttctccttaaggttgtcttccaggacttttacgaactcgtcgtcaaccttagagttagtcttctccgccagggccttagcaccagtcacgattgacttagcgattaccttggttgggaagagggtagccagaaggttgattgcgagggtttttagaaagataggcatctgaagctccttaacgttcaatgtctttgatcgccagtcgagtgctagcgaagtcagcggcattctcctcgttctggagttccataacagcccgttcgagtttctgaccccagaactgggaccgagcttcatccatggtgtacagataaatctgctcaaggacaccatagagataaatctgcggatacttcgtcagagcccaagtcgttgggttagcgaggcttagctctgggagtacagtccagtagttcacaatgaacggggcaccgtcaggaacaacggggaatactcgccagaagttacccaaccgagtgtagtaggttacaccctgaggttggtagttatagttaacatagtgggtgaagatatcttgggtgatgtactgaagagtacgcccaccgataagagagtcacccgtgatagaccttagagcaacaaagtgctcagggatctcaatgccaccaccgaaggccatgagagtttcgaagtgttcgttctccctcactcgtagcaatcggttaagacggtcagtagtgttcccaatgaacaacatcagaagttcttgggtaagatcctgacggtcagaccactggatagcagcaatagcgagatcagttacgttattgatcgtagccattcattacacccgtgcctcagaggtccgcatccgatagttatctcggtcattaagccaacgggtaaatcttgcggcatggtctgggtcacaaccaatcaggtttagatcgataggaccaccttcagccatgggacgattccgtagtgcctctacgacaaccagggggatacttgctaccttgcgcatattatccttgcggttgctattgacacccgagtggcgctcttcggcgttagcggacaggattgactcaacatcttgagtatcctttcggataaagagcccaaggtcttcgtcaattgcataagtcgattggatactcatgatgtacctcctaaggggaaacaaagggccccgaagggccccgttgggttagacctgggctacaacgtcgcggatcagagcaccggacttctcgttgtttacacgcagggtgtactcaaccagcagctggcgcttctcgctgtcaccggtcttagccagttcgtgctggaagaacggacgcaggtagcagagggcgtgcatcttcggatcaaagatgaacatggtgttttcgtggaaccagcggttggcacgaatggtgtacttaccgaagtcactctcgtagacatccacggtctgcgcaatgcggttgtccgaggcatccagggtgatctcagttgcacgacccttcatgttcttgctgatggccttcttgatcgagctcgaagtctggatcgagttagcctgaccaccgttgcgccagatagcctcagaggcattcaggagcatgtcttcggtcagaagacggaggtcaccagcggtaccagtgtcggaaccatcaccagttggcagggtaccgttagcacctaccgaaccgttggtcttgtagtaggcaaagatgtttgccatctgacccggagtagtggtgttacgctggatcttagcctgaggggcaccgaccatggcgtattccatgtccagcttcagttccttcgacttcttagccagctgatacgccagttcgttcttacgaccagccttcttgaccttatctgcggtaccggtgacttgcagggtctcgtccgagatttggcagaagttgttcagcatggtggtgaagctaccagccttgatggttgcatcctcaccttccactcgggtgttcttacccggctggcggagttcatcagtctgccactcgtgggtgatagcggtagctacgcccttgccgatagcagtcatgaacggggtgtcatagggtgcgatgttgtagatgatatcgataaggtcttcgcgcttaccgttgatctctacagtcgagacggcattagttggagttgccataagttacgtttccttctattagaaaatatcgagagaggagaagagagcagcagcggcttcaaccgactgatctttcctcagattagcgcgggcagctttaacccgcttagaaccctctgaggcttccgccctacgagcagcaggtttaactgcggcaggtagttcagtctcctccttcttctctagggcagccttgcgacggacctgagattcagcccacttacgtgctgcatcaagtacagccagttggcgggcatcagatatccctcggatctcatcctcggagtatccaatcgatttgccgtaggacacaatcttgtcaccccaagactcatccgtggtcatttccgggataagtttcttggctagctctgtctgacgcttaacgtaggcagagtggacaatctcggctcgcttatcctgcatagccttaatgtcattgcggcgtttaattaaggcctgggctcgatctcgggcttccagggcttccagtcgaagattttgatacttctctgggtcctgggccttaagctgctcccagtttacattgtcatactgattagcaccagcaatagcggtaacagcatactgctcaagctcggccagtagattagagcgttcagcatcaagctcttcgaatttagctgcatactgatcctctagttcagcttgtcgagttacgaactcttcattgcgaaggtagccactcttaagctcttcgaagttaacctcgtagacttcatccccaatcgggatctcgaagagtttatcctcaggatcttcctcggactcaacctcgggatcttcctcagaaccctcttcttcctcctcggactcaacctcttcggtgtcctctggagtaccttcgtctactacctcttcctcctcttcttcccctacgactttaccatccacggtttcatcaccgggggccaggaggtcatcacctagcaaatctccgaaggcttccgctgcctcgaattcatccatgccttgattctcaaggtccattacttatactcctttagagtgatcgaatccaagattgcctggatacgaagttgtacgcggttaagggcgtggagctcgtggtatatcgcctcccgagtctcactatctttgggggccgtagccttccattcgccctcgatctcttcttggacaatacggaaaagctcaggaagaacgttctcacgtaccatctgttgtgccgcatcagtcagcactagactatagccaaaacgttctcccaagattatttcctcactgccttagatggcttcttagtctcaggtactttaccgtctccgatataagcagcccgagcctgagtagcctcaagatgatactccgcttcgttacgagcccgttcccaagtgaaacgatcacgctcaagttgtagttccgcttccttaagggccatttctctctgctgaaggacagcctcttgcttcttcagttcgatctcagccagtcggatctgagcctctacctgcttcatctgggcctccgcttgcttagccaaggcgtccgattgggcacgttgggcatcagcttgggctttgatatcttcaggcttaggttgtgcttccttctgctccctgatggccttagcctgttgagcttcaggagaatccgggttagtccagaagcgatccgggtctttatacccagcgttctctgtgacttccttaaggatattgtaaaggttctgctcagagactaggaccccaagaccaccaccccctactacagcctgggccatttcccagatacgcataaggtggagcatctgctggtctttgttcatgttgccaataccaacggtaaccgtcaggtcggatctctctctccagttggcagggttaatagcaacccacttgcctcgtagctggaagacctcttcctgattctggtacttgatggcatggtcatgcagaagttggaacaaacgcttaacaccagtctctgcaaacatccgggcaatcaggtcaatctgttgctcagcagcagtcatcaactggttaacactcatagccgcttggttagagtgcagggtgttttggtctagtcctcgggtacggtcagtgatacctgtccgcttacccctgtctgcctctagacggtctagcataccgtagacttccccagacagctgaggggtctccagaggcatgatagagttcatggccttaacccgaacgatacccgctgcctcgttggtcagcaagtcttcgaggttaacctgtccatccaggactacagatcgcccttggttggtccggtagatgttatccatgatgttgcgcatgagcaccgaacggatctcttgaatgtctcggatcttatcgtagacactcatcccgtggaacttatgggcaattcgataggcattcaggtcagcgaagggacggcaatcccaaggctcgttgctgatgatgtagtcgcccacgtacaggatacggcgcaactcagagataccatccccatctacgtccagaagggtgtagcactcagaggcccatacctcacggttggcttcagcatcatccccagagttgtactggagttggccagtcatatcgaagttatcacgtaccaacctttctggctgactatcagagaattcatactcatcgtatggaagctcatctagtacatcctcgggaacacccaagagcctcaggtcacttacggtatacttctcacggtgacagaggaagcgtgcatcatcaatgcaggtagccaatcgatcaaccaggaagttctcaggcttgatacaggtgactttaatctctcgcttcttcttgtccttgcgaattttaatactgtaggttccatcctcatccacactctgtgctagaatctcagtgtctggatcagccaggatatccgctaccatttcctcagagagaccagagaatcgttcgaaggtaggattcaggacctcttctacatagacctttacaacaccggtcttcatcatcagagtgtcttggaaccagtcgaacattaccttgaacccctcgttcttacgcatgaagaggtagttcacatactcagtctcttgctctgcctgttcaacatcttcggcagtctgaggttcatacttaactacttgaccgcctgacgtgaataccttcataagagaaggcataatccagtctacagtctcttgaacatccctagatacaatcgcggacttacccggacgctcattaccgaagggctcaccgaagtaatacttcagggcttcagagcgttgtttagatagttccgacgagttgaaatcaagggcgtcgttaacaagttggtctagatgacgaagtacctgttcatcatccataggcttaatcttgcgacgacgcttagccattagacaatactcccaaaccaatcaggggtcaactttgcagtatcactcctgtagtatccactgtttcgtacagcaccaggacgtgcatgccgggaagccatcaacagggcataccgggtagcggatatcatatcgtcgtttctgtcgataatctttccgtcctttcggtggtacattttcatttcttttaggaagttcgtacacgtattaaacaccttcagatcaccattctccatacgggtcaacatccagttaacgccgaactctacagagttacctccgtgtttaccatcaggacccggtgggttactgaagggctcatacactacattgaggttgtggtcatctttaagaaggtctacgaatctacgaccagaggttgctccatcgtgcttaaaggcatcgtgggggacaactaccgggatctggtgaccacccttcaggtagatagcatcagcgtgcatccccaaggtctcaccactctcacttctctcatcatagaggtagtatttgtctttctcagggtcccaggcaacacaggcgatagcgttagggtggtcaaacccaaggtcgataccgatgatcctatggaagtgatcggggatctggaaaggctcacatacaaacttctcttccagaatggggaagactacaccagatccgagcataggaacaccctcggccctcatcctgcgttctgctggagagtatacagagagtagctgctctttaacttctggactgaggtgtggggcgtcttcccagcttgcatggacaaggaactgaccaggtttaagatcctggaggaagtccttgacgatctccgtcagaccatgctctggggtaaacgtcagatatacaatacccccagtagtagccgttcgggttacacactgggtataaatatccttggggcattcctcatcgagccagataacgtcgatggcagtacccatgaatttgtcctgggacatttcgtaggacttgaagataagggacgagaggcccccggaggcatgcttaacaactactgcttgcacacatccgggtttaccttccctacgaatcgtctctacgatatcttccttggggatcatccccgttccaaaagcctcagggttcttccagtcacctagtagttcggactgaagaatatcccgagtggtatccgtggagatccccgccgcccagcagttcactggacgatcatactttctaccagtccaccactcagggtagcgaccggtgaggtggcaggccatgataaaggccccggtgtaggttttaccacagcggttaccagtcatagccagcaactgggcgcagttcgaggaagctgcgataaacttctcttgccacccataaggagtgtactggttcatgcggaaatacttctgccgctccgccaactccttgactagattccgtagccgctcttgggtatccattagaccaccttgtcacctactgttgatgcacgccacagggcccgttggaggtcccttacgcgcactggagtttgtttagcccagaggctatcttctgcctcccatgcagcttcatcaaaccttccggacttcaggagattccacgtcttggggaacttcttcgtccacgctgtacccagctggaagttgacgctaacgagggcatcgaatagctctggagtgcaaaaaggcagctgagatacttgcccttgggctgcatcataggctgccttactatcttgctctagccaagtctctgctcgtgctagggtaatcggtccgtcttccccagggagttgcaagtggccataaccaccagttatcttcccgagggagtctttgtaccattttagtaccaacccctcccttcgcttgattagatcaaggtacttaagaggctgggaaagaaatgactttagccaactctggctcttcgtcgagaattcgtcgaacttcactgattagatcctctgcggacatatcttccacattcttagtagtaagctccagtttctgcttagctccgaagcctccacgatccaggatatcctgggccgccttgaggcgaatcccacctttctcttctgggttgctagcgatctctacaaccacacgaagcgccatggggacatgactcccgatgcgctcagagataaaggcgttgatgtattcggcatgctttcgatggtatgcagctacgttgcgttgcgcatgattaggagagaacccagcggctatatatgcttgggttttgttcataccatctgccagagcctcacaatagaggtccagcttttctttagcagaaagaggagcagccccaatcgagacaaccttttcgtctgacatacactgctccgatctctctaaatggtcaccccggtaggactcgaacctacgacctcctgtgtccaagacaggcactctaaccaactgagctacgaggagttaatggtggtcgaaggaaggaattgaacctctagcctaaggcgtccgggttacaaccggatggtagtagccatctacctctacatcgacctctgttggctgggacggcagggctcgaacctgcgacctgagagttaacagctccctgctctaccaactgagctacatcccaatgaaatcttaaggtaatttctcaaccttataatactattatatcatactttttctattttgtcaagtcttttttcttctttttttattttagtcgcaaggcccggttttgttagggtttttgctggaatgggaaaggggttcttaagactatcttaagataaccttaaggtaactaaccttcttagtaagtaagtcaaaaggaagaaggaatcttaagagcctataggtctattatactacattttggataccttgtcaagcactttttgatatttctttgataatcacatgatttacccgacgaacggtatgatagggttctatagatataccctaagactacccttcgtaggttgctggtaaggggtaagtggcatagggttcccttgatccaggttctataggtataccttaggactacctctgtttgctatgtcctggcctattgccccctatccagtaatataccaacaggtatcgggggttgcctttgatccagggcctataaccgggggataccccaaaagtctattttatcctccggtgtatctggggaatatccccccaggagtcccccagataaagtgggggtggcctatccacaggggtatagcccctagcccctccctaggataccatgggattagtccgggggtcaacccttagacccttgggctaagccccctagagctaccgttcatcgggtggacactagggtattcataagaaaaatcttagggtattcaggaggtggatcggggggaat

SEQ ID NO: 5 - DNA, 65590 bp, synthetic construct
  source 1..65590: mol_type = other DNA
cgattgcacaatcctaatagaaaaaatctatcacggacgttacctatctttaaaattaataaaattaatagtaatttggtaatttggtatactttggtatttgaaagcctcgcggcactaagcttgtacatatcccgtgaagtttccgattccagccagctcgcggcagggtcgccggaaacttccggacttccaatccatgggtcgcggcaacaccacggcggactaagcggcaagtgccaaaactcgacgaacggaaccggaaatttgggagcgcgacagaatcgctcagctggacatatttctaacattcgatttaacactcaatccaaacactcaccccatcgtcgcccgccaaccgccgactcgatcctcatccaccagcggatcgcccgtataacatcctataacaccacctaacactcattcaccatcaaacccacccagacctacagcccacccacaagcagcccatagacgcgatccctggccccatagtacaatcgcgccatactcagtgtcgcgacagagcaccaggccccatccaccaaccgagccaccgcgacgactccagaatcgaactcagggacgcagcaccaaatgaccaaattctacagccccgacgatctagtcacgccacaggaattcgcagacccgcatttcgcggcgatcaaccaaaagcgattcgacctgtacatcgacctgcgcgtccaaggttacagctcctggcgggtcttccgggccatctggggcgaggagcatatggacggtcccgcgcaggcccgcatcttcgcgatggagtccaacccgtactatcgcaagcagttcaaggccaagttgaacgcgaccaaaacgtccgatttgtggaatccaaagacggcgctccacgaactcctccaaatggttcgtgaccccaccgtcaaggactccagccgtctgtcggccatcaaggaattgaacgttctggctgaaatcacgttcgtggacgagtcgggcaagaccaggatcggtcgcggcctggccgacttctacgcatcagaagccgaggctcagaccgccaccgtcgctgctgcggccgaagccaatggctatgtgccggaaggcgaagagggagatttcccgtctccgacgccggaaccgaccgaggaagaccgcgccaaccccatttagacatgaaataacatcgttctaggcccgaatcggaccgaactaaggcgacggtagcgggaagggacgaaaaacgattctagggcggttctaggaagcagtagcctaaccctcagaaacgacaaagccccggactctagttcagaatccggggctttctttgggcgccttattcgccagcttcgatgatttcgaagttgtatttgacgccttcgtgctcgaaagtcattttgccagccgccttcagctgcatgcggaatcggatgtgcttcgaagaaggcaggccgaactcgatgaatgctgcgttggtggaccggaactcgccgcgcttgcctttgacagtcacagccacgccatggcgctgagtgcgtttcttggaaacttccgggtctttccaggagttggcgatggctgccgacaggtctttggtctctttggccttctccggcgcgttcttggcctcttcgcgcatcttccggatttcttccagcgcttcctcttcggtgatctcttcttcaggcttcagattctcttcctcggccttctcttcttccttcttcttggaagtgcgggttttgtagactttggccggggcttcctcttcctgaaaggcttcttcctcggccggcagagcattcaggatggcgaggcagcgacgctcagcagtcttgcggtcggagaagcgcttgacagtcgcatcggcgttgtgagcgttgtagaaggcgaccagttctttcatttctgcgttctggatgtcgccgaaggttttgatggagttggtcatcttgcgatcctctgttttggaagatttcgttcgggcttcggtttgtcgccccgttgaaagagattatgcctaggtcgattctgcgtgtctacatttatttcatcagattgatcgcaaagccgacgaacggttgtctgatgtgaaaacaccgcagagcaggctgcggtgtttgttggcgagggcgatggtcaagcgaacggatcgcccagcatatcttcagacggggcacgttgcatatcgcccagcttgtcttcgtcagttgccgggctggacgggatcgcgccggaccgaagcgacagagattccgactgcctgctcacctgcggagccggaggagcttttggcgcagtaggggcaggcgggaccggcaggcctggaggagccatcggcgccatgggcggttctggaagatcaggaagactcggcgctatgccgccaaaggaccgctgcgactgctgcgccggattgtcgccgaacatttcggcggatcggttgtccttgatgacttgacctatggccgtcttgtcgccgcccggctctgccggatcgaagcccgtgaatggagtggtaacgcagcgcatgttcggatgggtaggcagatttttcaggactatggagtctgcccagacatacggattcttgtggttgccgggtccgtggctgatgatctcggcaggaatgtggccgttcatggtgagccattccgccatcccctgccgggtcagcgggcttactacgatctggccagtggactcttcggcgacggcgaacagatcaccgagcttgaatccgatgatcatatagttggacctggactcgccgacgaaagccagggtttcgatgttgtggtggatatcttgatcttcccagatgtacagcatggtgtcactcctctttggcgaaattgttctttcgaaggtagtaggcgatcttcagtcgtggccagaacggcgtcagtgccacgttgtcgtgacggatgatcgcaaagtggtccgttccttccaaccctgcgatgaagcagcgttcccaggcgctcccgctgtagcgatcatctggcttccagacgttcatgcctgagcttagttcgctcgactcccatttatgatcgatcttgacgttcctcatggctccctcaataaggctgcttgttgtgctcgaccagagacataccggccggaactttgaagcggacgtattcgatcatcatcgccgttcgacgccgattcgtgccagactcgtcattaacgaagagttcgcactgatgcgggatgactcgcgtcaccaggtcttcgcctttcgaaccgtagaactttatgtaggctgcgacgcctcgcgtcatgttcagatggaccgtttcgcagagtcgctgtgcggccgacgcgctggacagatcggtcggactgaccgtcacccagtaatggccactttgttctttatcggacatcaactgccctccaatgagaaagcctctgccgagtcgcagaggctggttgttgttagtcgcgcttcaacagaacgactttgtcgtatgcgcgatatttaccgcgccagtcttcccagtagtcgccggccgggcaggtaagctccagggtgatttcgtcgcgccagcattcgacggaaaggacaattgccttgttcctggttgcggaatccgctctgagcttaatcaggatttcgtcgcccgttttcagctcatcaactctcacaactttggccatgacacaatcctgtttgaagaggcgcggccggaaccaacccagccgcgccgatggattaacgtttgtgaaggatggacacggtgtccacgtcgaggatgctgatggtacggcgacggcgcggattgctgcgttcatggatgcgaatcaggttcgcaactggagcttcgtccaccacccatacatcttcctgcttctgcgtccggagcaggaccgttacttctgtgttctgggcgaggccgttgcagatgaaagtgaacttggacgagggagttctgtacatttcgagattcctttttggacttcggggtccgacttctcagccggtgaagagattatgcctgatcatcaccatcgagtaaagcacttgtgtaccaattctccagttaaatggaaccaaagcgcggtatcgctgatcgctacgcttccacgccatggtccttcttgcttacagacttcgaaccacaagctgatttccattctggccaggactcggccgaaggattcgagccggcgccggctagccaggatgtcgacattcacccgccgattgatttcgtatcggcgaccgttgatgacgaagaccaggcgcagggtgcgtccgtccagacattcccagtatctggtttcataccgaagccagtagcgcttaccagccgggcctaccatggtcatttaatcgatgctcctggacgcgccacgcggtccgttcggcgggacgggatcgaaccgccccaggatatagtcgggccggcgcgcttcctgaggacacacggcgagaaggcgccatcctgcgtccagggcgtactggagttcgtccgtgcagcagtcttccttgagcagcagacggttgatgctctggagattcggaccagggatagcagagcctgtgaccgagttccagccttcgataccgtgcaccatctgcggctggttgatgtagccttcggacctgccagccaaccggctcgcggccagctccaggcgctccagcatgggtcgcagagcggcttccgggtcaacatcgtcccagagcaggaccaggttgatgatggtgtacggatagtccttgtccaggtcccaggccgaagcggtcagacggcccaggccgatttcattacacatcacaggaacgtcgttgctccaggtggacggctcccagttccgctcgccaggattcccgattgttacgccttccaggttccccaggagaatgtggagcttgctgacatattccgcctccagcgctttccgctcttcgtcggtctggttgtggcgatagaaggatggagggctgacttttgcatggtagagtttcatggcggttcctcggttttggaagatttgaaagttagagaatggtgtcgcagtatttctcgaaaggactctggcgcttcttctcgcagatcgcgcaggtgatttccaggtcgggcagctcgctgtaagtcttgccgagatacagccagcgcttgcacagactgcggccgtccgacgtgaagaagtggactttgcgagcattgccgggttgcgcccagccaccttgatcgtttttgcgcttgctcatggcgatatttcctttggatctggaatccatgccgtccgttcgctcacccaaactccgtctacatagatcaggactgacagagcagtcgcgccaaacccgaagcctacatagaatgaaccgggatcgatttccatggtggcgctattggtggcgctattgaaatcgacttcaaagttgaacatcgaatggccactcataccatcaccatgtcgatttcattgatgaagaaatgaacatcgacaccatcggcgcgaccggtgtaacgcagccgagtcgtcccatcaatgtgggcttcctcgacgccgatgacttccaggatggtgtcgttgcagtaaagcttgacgaacatcttgatgccggagccaatcgccgcgaaggcgatctgcttgtacaggtcttgcttgatcacgctttgcgctcctgtttgcggatgtattctgccctggcttcggcttcgaacttcgcccaaagcgcgttgtcgactggaccccagggaccggtctgagtcccgtccaccggcgcgaacttgccaccggcgcgacgatacttggacagagttctcaggctgatgattttctcttcttgggaatcgagttggacgatcacttgacatacttcccgaagtagcaggtgtggagtatttccatacccttggagatagccttcagcttgataccgaaattcttgatcgcccagtccagatcgtggtgatagaactcgatgatgttgacgatttggccggcgctgtagtggatgacgcgaaccttgccgttccagcggaaatcttcgcggctgtttacggcctcgacttcgatggtgtcgtttctgccgatggtccatttggagctagtcatgtcgcaatcctctttttggagtgtttcgcgtttcgatgaggtaactatacctcagtcacctcatcgagtaaagcacttttgaacagattatctgaaatttctggaagacagatccatcggctgattcagtcaatcaatcggcggaaccgagacgccacccgttgcgggcgaaggccgagcgaccagacagcttgcgacactggatggagctaccatcgtcgaaggtgtaagatacggcccagtccaggcgctcgcgacgtactgccgcggcagcgatatcttccagacgggctttgagaagttgttctgctttggtcatttcaagttcctctttggagcgcttctggcatcgatgagatgactatacctaagtcatctcatcgagtaaagcacttctgaggaattatctgaaatttctggaggccaggaattgtcgccagagccagtcaacgtgatcgttgagatagcgctcgccatcggacgacagagccaggcagccgtcctttatgccgagcttggagccggcaatccattcgaactcgaactggagcgttcctgggcgaggggtatgggacatgatgtacttttgcacgatcaggaatcctcgaaaggtttggcgttcgcgatggccgcgattttctgcatgccgatgcgatagaattcggcctcttccctggccttggccagctgctccctggcttcgcacgccagcttgtgctgattgttggcgttctggatgcttccgtccagactcctgtccagttcttcccgaagagccttctgcgacgccagctctgcctccaaatcctggattctgagctggagctggcgattcgtctcggcaagcttgttttcgtgagcaatggagttctgaaggcattcatccagatgagctttttcgagcttcagctggttttcgcgatccgcgtacgcagtaagacatttggtcagacgagcctgttctttcacaagatcgactttgcggagcttggccctggccagagctttgcgaagtgactccagctcaagcgggtgaagatcagcatccgggtcttcacgctcttccttcggctccatccgcaggtagtattggccggcagcgaaggccaatagggcacggtcggaattctctacggaagcggccatgaagtgggaattataacgaggcagaaacttgcccgcctctgggtcccagcacaggctatccgccataggccagaatttccggaagttatctgcgttcacgcccaccttgaccgtttccggcgcacctgaatccgtcgccacttccaggttcaggtgaatggtgccggtgatgtactggaagtacactatgctagacataacacaaactccatgagaagtcgcggccgggaccatcctggccgcgcatcgtgattactcttcgccttcgtccgcgctcagccactcttcgaaggcgaaattcactttcgactcgacccaatcctgcagctcatcggcgaactcgtcactgtcgatatccatcggaacgcccagagtttcttcattccacaattcagcattcagctcgaactggaccatttgctcgccatcgacttccatcaggatgcggtctgcgacttcatagtcgtctacatcgaacagatggccgccagtcaggaggtgctggacgaaagcttcatcgagattggtgacttcgatctggacttgtttggtcattttccgtctccttacttagcggctttgaattgggctttgaggttggccagttcagctttcagatcgatcatcgcctgaccgcgcaggccttccatttcggcctcgctgattgcgatcttggctgcgcggattttgtcgttgatttgatctttagtcatttcggcttccttttgtttgaagggtttcgcgtttcgatgaggagattatgccgacagtcagactggaagtaaagtgtctgatcgaagaaatttctgtacaccgacgaacggttgcgctcgaccgtctgctgcggtgtcgatatactcggcctattgcgaacaatggactgattaaatgttcaagctcaatcctgcactgcgagcggtctggcgaactcgtgcccgttacaaagtcatctatggcggccgggcgtcttcgaagtcgcacgacgctggcggtatagccgttttcctcgcggccaactacaagctcaaattcctctgtgctcgccagttccagaaccgcatcagcgaatctgtctacacattgatcaaagacaagatcgaaaactcagagtataatggcgaattcatcttcaccaagaactccatcaagcacaagattaccggctcagagttcctgttttatgggatcgcccgtaacctgtcggaaatcaagtccaccgaaggcatcgacattctctggcttgaggaagctcactacctgacacaggagcagtgggaagttatcgagccgaccatccggaaagagaactctgaaatctggatcatcttcaaccctaacgaagtcaccgatttcgtatatcagaacttcgtggtcaaaccaccgaaagactcctgcgtcaagatgatcaactggaacgaaaatccgttcctaagcgagacgatgctcaaagtgattcacgaagcctatgaacgcgaccgggagcaggccgagcacatttatggcgggattccgaagactggaggcgacaaatccgtcatcaacctcaagttcattctcgcggccatcgacgcccacaagaaactcggctgggagccggccggatcgaagcgcatcggcttcgacgttgcggacgacggcgaggatgcgaacgccactacgctcatgcacggcaacgtcatcatggaagtggacgaatgggacggcctggaagatgaactgctcaaatcgtccagtcgcgtttacaacctggcgaagatgaaaggcgcatcggtcacttatgactccatcggcgtcggcgctcatgtcggttcgaagttcgccgagttgaacgatgccagcccagacttcaaactgatctatgacccattcaacgcgggcggcgctgtcgataagcctgatgacgtctatatgaagctgccgcacacgacgatcaagaacaaagaccacttcagcaacatcaaggcgcagaagtgggaagaagtcgcgacccgattcaggaagacttatgaagcggttgagcatggaaaggtttatccatttgacgaattgatttcgatcaactctgaaacgattcacccggacaaactaaatcaactgtgtatcgaactttcgtcaccgcgcaaagacctggacatgaacggccggttcaaagtcgagtccaagaaggatatgcgcgagaaacgcaagatcaagtcaccgaacatcgctgactcggtgattatgtcggccattctgccgatccggaagcccaaaggcttcttcgacttctaaacatagaaaagcccggatcgctccgggcttcgggtcttactcggtgcggttcctggcgctgagtgtcgacgcaacggcctcgccgactcccagggcttcctggccggccgcgagcgcttcggcttccgactcgacgatgaaatcatcaccttggccatcgcctggtggaacctcgaccagcacggcttcttcgccttcgaaccgcaggtcataggtcttttcgacggacagaccgtaacgagcgttgagcgcgtcccagagctgggcctcataggtccgcaggtcttgcagagctttctggtggctgagcatggccatgtcaacggcccgttgcagggtttcgtccaggacgttgaaccgaatgcgaagagaacgaatccgctcgacgacttccgcatccactacatgtctttcgatcattacttttcacctttgctgaatgtaacgttgtagccgttgtcggccaggtaggtcagggcgccttcgaaggaagttccgacgaggtgcttgagcttcatttcgcgttgcgcggccaaccagaatgcagttccggaaaactctgcgcggccttcggacaaaaccctgccatcaggcccggcgatccgtacatggacggaagatagcttgatcggcatcagtgaatacctccactggcttgcgacggcatgctttcggcgcgagcggcttcgcagtccgggcagggacaggcttggcggacgcgctccaactcgtcggcatccatgacatagagcttcccgtcggcagtgtcatgtgccatagcgatgttcgggaagtcggttgcgctcaggccggcgacagtgcggatttcgtcccagagcaccagatgctcggcattcagtcgggcggcgagcgcttcgtgctctttagcgacgcgcgccatgaactcgtccatacggaatgcgaattccgtatcgatggcacgggccgaggccatggagctgaggcgaatcggttctttcttcatgatgattctcttttggttggtggtggctaatgtccgtgcctattcaaggccggctcaatgcggcgaagaacaacacgatgaagaggagcgaaaagaatgcgaacgccagagatattccggaaatcagctttgctatgccctgcatctcagtcgccttggcagcgagtctggccttagcctcgataacttccggtcgtggcgactgaacatattctaaggcctcttcccgcgctttttcggcctcgaccaacctagggtcgcgggccgagacttcgctgtgccctggcctcgcgggatgggcttgcagcgatggagggagttcagccgccacgactccatagtctgcgcaggcccaagcgatcccgatgaggatcgcgaggatggattggacgattcgcagcatcactttgtcgctagggaagttcatgatcaatcctccaccgaccgaacgatttccatgttacgcccggcattggtccctgcattgaaggcgcgccgaccgtcgctatcgtccagacgcatgagcttggtgacattcgacttcttgtagccaggatcgccgaaatgttcgtgaaccgcagcttccttcaccacaaccagagacgttccggcagaagataccagctccatgcgcttcctggtgatggattgaaggcgatagctgatttcctgggtcgcggccagcttgaattgcgcggcaacctttacgttgaaacgttcgtacccttgagccttctgatactcccggcacagacgatcaatggcctcgaccagggagttgaacatgttcaccgccagctcaacgtccgacttgtagcctttaaagcgtacggcatgaccccagcgcttggtagtgcttccgtcgcgggcgctcctggatgccttagccgatgctctgtggttgttgatgccaccggcgaaatccatgatgcagtcattgtacgtcgccacggccactgagaaaaacttcatccagttcgggattgcggaatagtaacgagtcgcaatctgctcgtcgaactcttcacgaatctcgccggtcgcttcgaagccgtggaggtcatacttgtccttcagcttcttcacgcgctcggctgcgatggccgcttcgtgcgggctggaggagtcggctgccatggcggtcagtttgcggatgcggtctttcgccttctcgatggcttccggggtgaattcgttctggtcggtcatggtcggttccttttgtctgaagggtttcgcatttcaatggagctattctgccttcattcagaatggaagtaaagcactttcttccactatttcgtcatcgactggaagaaattccagatccaatcacctgctaccagcaagaggatgagggcggcaaagaacaagacagcggccgcgagctgtgcgcccggcttcagtttgggatggctgagcttgggctctacgggaacggccggagcgctagcgctgcaaccggcgtctcctgggccgtagccgatgccgcgttcacacgcctggagcgacaccagggatttaagatactcggtctgcttttgcgactcttcgtagatgccggcgacggcgaaccagagggcgaacactacggacgtacacacaacccaggcgccggtcagcaggagggccagcgggccgatgacgaagacggtagcagccaggacgattgcgccgccccagatgatgaagccggccagaccattagtgatgtcgacacagaactttttcattttcagattccttcggttacgggatggaggggatttgaaactctgcgccgccgagaacatcaactattacgaggatcgctcggcttgcctcggcgaaatgactcacgaaggttatgagcattcttcggctcgccaggcttgcggcggtcgcggctggaatctttgcctctggcgcagacatacagagcccacaggattccagccgcgaccgccgcaagcctggcgagccgaagaatgctcataaccttcgtgagtcatttcgccgaggcaagccgagcgatcctcgtaatagttgatgttctccatcgatccaaccagcaccagagactcgaaatcgaatcgatgatcgtggatggccgagtgattgaagcaaagccgacgcggcagctccgggtgccaaacatggaggcgaccggccggaagttggacctgaatgaaccccaggccgtgcagagtgatcttgtccttcatcgggtcatggacggtgctcatggataatcctcagtagcagaaatgcattgtgagagttacgattgacaagccggtcgcccatagaagggcgaaccaggccatggctttgatggtcgtgtagatcatccgaagaattttccggcacagatcgggccaatgcccatttcgatggatgcgtggttggtcaactcgcgaccgcagcaggagcactgaccagtcttccgaccgtaggcgactgccgattccatcggcttttcgaacatcttcagaacgtcgtcgtgctcagtgtcggtgcagtcgcggctcttgatgaatttgccattggtgatccggccgaggtagatgtctcccaggacatacagactcccggcgttccgactgttagcgctagcctctttcacaacaacgatgagcggctcctcgccttcgccagccaggcggattttcgggcgcttgattccagactctttcgccttctcaaatgctttctcgataccagaaatgtccagagtcggcgcagcagcttcctgcgcggccactttctcgcgatacttggcgaagttttcgatggcgcgcttcgcagcagcgatctgattttctgtcaaggagccgtatttatacaacgactcctgaaggctctgagcgaagctgaaggaatttccagtccaccactcgacgatgtccgggtgcgcggcttcgaaggccttaattttgaggtcgcgctcttgcgcagcgctacagattttctcgatgcgcttttctgccgccttagcacggctcttggcgcgctgctccgggctggttttgtactctttgtatccgacgccgccgcaggcaaagcaggcgcgaccataagacgaagggccacggtacaggccggtgcctgcgcatttggtgcacttgtcgcgatacagcttcacttccttccgggagttcgggcgggcacccattgacacgtcttccagggtcttcggcgcttcgttgttgatctctacggtagcgaagtcatcacccaggtcttcgaagccggtgaacagattctctgctgcgttcatatcgattctcctgtttggaaagttcgtttcgatgggttgactatacgccagaaatggcaaaacggtagcactttcgcactaccgttcgtcgggtcgcagacgatcaataaatatcgctattgatcttgaatccatgctcagcgccgtcgttgtagtcatactcagcatagccgtcgcagtagtcgttcaaatgctgaatgatgccaagaacgtcgttcgcggctttgtgtcgtttcgcggcgatggtcttggcgatgctgtcgccgacttccagggtttcggccgtccggcgatggatcagcaggcgactccaaagatagagccggacgcgacgaataatgtcgcgatggcgctccagtcgtccgtgcaacttttcaatttcgtgctcgcgagattttaatactcgtcgaagccgttgaacttccaactccaaatcggccttagtagccatgttcacctcagaaaggaaaatcgtctgagactccaggaagctcgacgattattgttgagcctgatcggtctagaatgcatccgaccgagccagccctgtgtggatagggtctacacgaactataacaggtgacttccatgactattcgccaggtcgatcctccacaccatctgcaaattggcgggcgcttcgacttcagttttcgttcgtccaggacggcgcgtctgtcgcaggaaaggcagcgtgccttaaccgacgtaaccatgggtatagtcgatcattcggatgagctcatcatcctctgattctctcgacttcagaccgcccaaagagtccgactggccgtcgcatttcgcatcgatatccatcgaagacaggaatcctgccgatactgcggtgatatcaactttgtcgccggccttcttgaagccttcgcactcagcatcgagggccacaattgcactcgctgccatgttcacgtgactgatgaggtcggggaagatcacaggaacttcacgcgacatgccacggaccgtcagcttcaggactacatacttcatactcactatcccttttgtatgtgaggaaagaacttgctgttttccggatgatggaagcgctcggccgcaggcggtctttcttccggacattgaatcgtcgaaggagggaaaaccgcgccggcgataatcgccgcgaggagtgcagcggatgtcgacatccagagggcggtttccaggctcacccgaacttccggacggcgcggcttcattcgctccaccctcttcccggttcataggatggtagatcgccgccgcgaacccagttgtcccacatatggttcagaccagtcggtggctggggcggcggactgtagaatccgggagtctcagtgctgctcaggaaataaggcgtgcaaacggcctggacgaccaattggtgccgctcccacatcttatcaatcgctcttagcatgacgtcttcgcactgagccttactgtcgaaccgtctgctagtatggtccggcatctggacgcagccgtctccagtgcaaaggaaagcagtagcaatccatacggtgatactcgccatttcgtcaccctctttagttgatgagcagagtctattccatctgctcgccaggagtaaagcgcttttcgtcgggataaatgccgatgatgtctgcgtcgagcatccaaatctccacggacggatcgtcgctgctgatttggtacaggttgtacagctcccgtccgccgacagcgcgctcgcctcgcggctcaaccgccagaaccttgccgtgcccttctccgtgctcgtccatgtatatgacgtgatcgccgacttcatagctttctttcgtgacgaggcgtgaacgcgagctgttctgcgattccaccatccaggaatccagcacgccgcctttcatgtcgcgaaacaatcttgccgatttgtcgcaatcgaatacgcccagaacttcgccgtctttcaacacgatatgtacgattggcaaaagagtattcatgtttaatctccattggttgataattagagtctaatctgccgaaaagttcccgtaaagaattattttctcataactgattagttgcaactgttaatctgatgtatatgtttgaatctcttttgaacgtttgatgtttcccctataataagcgcacacagccaacaaccacgtggaactacaatgtttaaactttcctggatattcgggcgcaaaaaggataatgctgcctgttctgaatcggcgccggagaaagtcgcacaaatccctcaacacgatccgctcgaccccatgatcaagctgggaaggattcgcggctggaacgtcgagccggagaaagccccggtcattcgtagtgtgaaagatttcctggagccgggcctatctgtcgcaatggatagtgcgtatggtgatggaccaaccccggccgcgaaggctgctgcgggcggccagaatccctatgtcgtcccgactatgttgcaggactggtacaattcccaagggttcatcggataccaagcttgcgcaatcatttcccaacactggttggtggacaaagcttgttccatgtctggggaagacgcagcacggaacggatgggaactcaaatcggacggcaggaagctatccgatgaacaaagtgcgctgatcgcccggcgcgacatggagtttcgcgtcaaagacaacctcgtcgaactcaacagattcaagaacgttttcggcgttcgcatcgcgctgttcgttgtggagtctgacgatccggactactacgaaaaaccgttcaatccggatggaatcacgcccggctcctacaagggaatctcccagatcgatccatactgggcgatgccgcagctcactgccggctcgacggccgatccgtcttccgaacatttctatgagccggatttctggattatcagcgggaagaaatatcatcgcagccatctagtagtcgttcgcggaccgcagccgccagatatcctgaagcctacatacatcttcggcggtatcccgctcacccagagaatctatgagcgcgtgtatgcggcggaacggacggcgaacgaagccccgctgcttgccatgtcgaagcgaaccagcaccattcacgttgacgtggaaaaggccatcgcgaatgaagacgctttcaacgctcgcctggcgttctggatcgccaatcgcgataaccacggcgtgaaagtcttgggtactgatgaaagcatggagcagttcgacacgaaccttgccgacttcgacagcatcatcatgaaccaatatcagctggtcgctgccatcgccaagactccagctacgaagctcctcggcacttctccaaaaggattcaatgccactggtgagcacgaaacgatttcttatcacgaagaactggagtccattcaagagcacatattcgacccacttcttgaacgccactatttgcttctggcgaagtcggaagaaatcgatgtgcagctggaaatcgtctggaaccctgtggactccacgtccagccagcaacaagccgaattgaacaataagaaagccgcgaccgatgaaatctacatcaactctggcgttgtgtcgccggatgaagttcgcgagcgtctgcgcgacgatccgcgttccggctacaaccgactcaccgacgatcaggcagaaaccgaaccgggcatgtctccggaaaacctggccgaattcgagaaggccggtgcacagtcggccaaggcgaaaggcgaagccgagcgagccgaagcccaggcgggcgccgtagagggcgcaggcgccccggttcccgccgctccacgcgggactaagcctctcgcgaaagcggccgaggaaggggccagcgaggccgctgaaccgccgtcgcggccggaccccaaggccgagctgcggaacttgttggtcgatcttttgtcgaagctccaagacctggacgacattaaggcgccggacggcgtagacatagagcacaatgatgcgcctggcttaaagcgcacgtccaaacctggcgtgtctggcatggagccttcggtgttttcgtccaaccgcatcgtcgggcctcgtgatcattcggaactccagagaatcaaggttaatggaataaccaccttgatcgaaaatcctcgcggaagcattcggcaaggaaaggatgggagttggcgagttcagatgaaacaccactacggattcatcaagggaacgaaaggcgctgatggggatgaagtcgattgcttcgtcggtccgaatctgggatccaaacgggtcttcgtcgtcaaccaggtgaacaaggaaggtcagtttgacgagcacaagtgcatgctcggcttcaacaacatcaacgatgcgaagtctggatatctgtcctgcttccgcccaggttgggatggtctcggctccatccatgaagttgacctacccgctttccgtcgctggctggcgaacggcgacacaaccaaaccgtttggaggcgagtgatggcattcaaggcctccaaaaagcgcgaacgccgggcacctcttccggtcggaagagggaagcccataattccatcggcaggaatcgaggcctggtatcgaaagcagatgaaggatatttccaagctcatgatcgccgactatcgaagcgagattgagaaggcactgtcccagcctgcggccgaacggttcttcgccagcgacgaatccgttaacgccctgttcaagatgacccttcgaagcctacagcagcgatggaaccgcatttttgaaggcttcgcggccaagatcgccccggagtttgtcaaccggaccgaagaagcggccaccgccgcgaccctgcacagtttgtctttggccggcgtcgatcagccaagagctgcgtacaatgagagcgtcaggaataccctggaggcagcaactacttacaaccatactctcatcaccaggattcaagaggaagtccacgagaagatttacacatcagtaatgttgtctctgacttccccaaatccggaagagcaaggaacttccggcataacgaacgcacttcgcaaagtcgggaagttctctgaagatcgaatcgaactcatcgcaagagatcaaaccagcaagctttacagttcgctgagcgatgagagaatggcggaaaatggagtcgaggaattcgagtggatgcactcttctgccggcaagactcctcgccatacccacctggagaaagatgggaaaaggttcaagctgaatgaccccagactttgggagggtccgaaggccgaccaggggccgccaggatgggcgattaactgtaggtgcagaaagataccagtcatttgacatcgataggagcgctatatgccgttagttcatggcacttccaatgaggctcgttctgaaaacatcaagcgggaaatcgaagccggtaaagacccaaagcaggctgcggccatcgcttattccatccagcgcagtgagaaagggaagacggcgaaagattgttcgcctgagctcgtcgccgatcttcgcgccatggtggacgctctgtcgaggctcgtgaaatgaaccgcaagacgtgccggcgccgactcgtggtcgatgtaatcagggccaatattcacggcggattcttcagcctgaagtttgccgccatcgatttggcaatcatcggcgtcgccatcttgatgacttttggccgataatgctgagaaaatctggattctgactaaaaattctagtccggatagccgcaagttaccgtttacggaaaatagcagtaatttggaaagcctactgccgcgaggctttaacagagccagttcctaatttccgatttagccgcatgcttcaaaagtatatagcctgggaaattagaagtaacgttacaatagaattcatctataagtaacgttataatataacgtcaatctatatgctctagacgtattgaaattcaatttttaatcgggaaattggtaatttgaattagtttaggagttgaaagcctcgcggcagtaggcttggacaaatcccgtcaagtttccatggtcaaattactggattttcgcggctgaggggctggtaattagatcataatacaaattataatgtaagataacagtcgcggctacatctaattattgttccgcttatttacccttagatgtcctgcgtatataatacagccatagtccacgactcttcgaattaacgatgacaaagtcgaaaagaaaaattgacgaaaatggatatatgaccatcgagggctgcccgatcagctcttatggcgttttccagtattctgctggtcaactcggtcttccgggcgatccgacgcggattgtcaacgtatatcgcccggagtctgccgtcagcgatcctgagtacatcgaatctctgaagaatctcccgttgatcgacgagcacgaaatgctgtcgggattcgacgacgatgacgacagcgtggcccccgaagacaaaggggtggagggtatcatcacctccaacgcttactacgaagccccatgggctcgcggcgatatccgcatctattcccgcaacatgcagaatcagctggaaaggggcaaagaagacctgtccctaggctatagttgccgctacactgagcaacccggcatctggaacggaacgccttatgaagtcgtccaggacaagatgcgcggcaaccacatcgccctggtaaaagagggtcgtgtgccgggggccagagtattggatggtctgtgttttgaccatctcagttttgatttcagaccatccgatgagggtaatgaaatgagtctcaagaaagccaagcggaagccccctgtccagcgcgtagggcaagctgctgactcggcggtcgaagagttgcgcaccctgtggccgaaactctctgcgtctgtccagaagttcctgggcgaagaagagcaggagccggagcatcaggaaggcgccgctccggccgaaccgaccgacagcgagcacctgaccgagcatccgactctggaaggcgcccagaaggacgacgaagagcaggaagaggagccttccgttgtcgatccggccgtggccgccgtcgagccggagcatcaagaaagcgccgcatccgaaatgtccggtgaaggcgaagtcgccgaactgatctctcaggtcaaagccattctggctcgactggagggaacggtagccgaaggggcagacgaagagcatggcgaaggtcaagatgtcgtcgagggtttggaggagcagagcagcctcagcggctcgcaaaccgccagcgacgatggtggtgagagcaaggataacagcgaggaacttcctgaaatggcacagaagaacgcgcaagatgctgcaattcgcggtctctatcgcgacattgctgctaaagatcgcctctacaagcgtcttagctccgtggttggtgcgttcgatcaccgagctatggactcggctgaagtcgctgtttacggcgtgaaaaagctgaacatcagctgtgcgaagggccaggaagctctggcgctcgacatgtacctgaaaggcgtcgaagcctcgcgcggcgcggccagccgtcaatcgaaagcccaggattcggccggttctgctccgcagtgcgccgagctggacagctacctgaagggggagtaactcatgttccagaaacaagtttaccgccagtacactcctggttttccgggcgatctgatcgaggacggcccgaagcgggcgcggccgggtcgaatcatgtctctgtctgccgtaaatccggctgccaccgccaccggccccaaccgcatcagtcgcgctttcggttacgccggtgacgtcagcgccctcggcgaaggtcagccgaagaccatcgcggctcgcgcttctgaagtcgtgatcggcggcgccaacttctttggtgtcctcggtcatccgaagcactatgcgctgttcggttcggccggagactccctggctcccagctatgatctgcccgatggcgccgaaggcgagttcttcgacatggccaccggcctggtcgtcgaaattttcaacggcgccgcaaccgccctggacctggactatggtgacctggtcgcctatgtaccaaacaacctgcctaccgccgacaacgcgcttggcctgccggccggcgccctggttggcttcaaggctggctccatgccgaccggcttggtccagattcccaacgcacgcatcgttaacgccatcagtctgccggcccagtcggcggggaatctggttgctggcgttaccatcgtccagctcacgcagtaaggaggcgtcatgagccagatcagcaagacccattcgcgcctcgcaggccgcaatgcgaaacctttcgacctgaaaaacatcaccaatgacgccgtggcgtctctgcgccgcatcggcctggtattcgatcacgccgtcgtccaggaccagatcaaggccttggcgaaggccggcgcgttccgctccggctcggccatggacagcaacttcaccgccccggtgaccacgccgtccatcccgactcccatccagttcctgcagacctggttgccgggcttcgtgaaggtcatgaccgctgcacggaagatcgacgaaatcatcggcatcgataccgttggctcctgggaagatcaagaaatcgtccagggcatcgtggagccggccggcactgcggtggaatacggcgaccacaccaacatcccgctgaccagctggaacgccaacttcgaacgtcgcaccatcgttcgtggcgagctgggtatgatggtgggcaccctggaagagggtcgtgcctcggccatccggctgaacagcgccgaaaccaaacgccaacaggcggccatcggtctggaaatcttccgcaacgccatcggcttttatggctggcagagcggcctgggcaaccgcacctatggtttcctgaacgatcccaacctgccgccgttccagaccccgccgagccagggctggtccactgccgactgggcaggcatcatcggcgatatccgcgaggccgttcgccagctgcgtattcagagtcaagatcagatcgatccgaaggcggaaaagatcaccctggccctggccaccagcaaggtggactacctgtcggtcaccacgccatacggcatttcggtttctgactggatcgaacagacctatccgaaaatgcggatcgtgtctgctccggaactgtcaggcgtccagatgaaagcccaagagccggaagatgctctggtgctcttcgtcgaagacgtgaacgcggccgtcgatggaagcaccgatggcggcagcgtgttcagccagctggtacagagcaagttcatcaccctgggtgtcgaaaagcgggcgaagtcgtatgtggaagacttctccaacggcaccgctggtgcgctgtgtaagcgtccgtgggccgtggtgcgctacctcggcatctaaccgatgcctattcaccaaaggccgggtttccggcctttgttcactctgactctgactcggttgtaggggccggttagggcataattactaggactacgccaatgactgtttacatcgtttccgccatgactcaatccgtgtcttacaatgcgtatgacacctctgatccgtccaatcctcgccttcagcggaagattctgattcgtggccgcgccggcatcgcatcagaaacttccggcttcggcgacatgatttccgacgcggccgggcgtccgatctggaccccgcagggcgattgtactgccgtgagcgattcggatttcgagctgctccaggccaacaagattttcatgcgtcacatggataagggttatctgcgagtcgtgaagaccgacatcaccagtgaccaccagcggatttccaaagagactcgcaccatggagcgcgatggattccagcctctggacgctgctcgtctgcagcagaaaatcaaggtgactaccgccagcgcttcccaggaacaagagttccggatttaaccgagggtttcggtatggtgattttcgacgaacataagtttcgcacgctgtttccggagtttgctgatccagccgcttatccggacgtgcgcctgcagatgtatttcgacatcgcgtgcgaattcatttctgatcgcgattcgccataccggattctcaacggcaaagccctggaagcgtgcctgtatcttctgaccgcccacctcctgtcgctgtccacgatgcaagttcagggcgcggctggaggtggcgtcactgccggtgggacccagggcggtttcatcaccagcgccacggtcggcgaggtcagcgtggccaagctcgcgcccccggccaagaatggttggcagtggtggctttccgggacgccttatggtcaagagttgtgggcgctcctcagcgtcaaagcggtgggcggattctacatcggcggtcttccagagcgccgaggcttccggaaggttggagggacgttctggtgatccctggtgcgaatcttctgcgaatggccttcggggtcatcggtactcaaattgtgaaatatcgcaagtttgagcagcgagtgaagaatgatcaagctcagtacgtttccatgttcgaggagcctttcgacctggcagcgtctgttcagcgagtccgacgcgatcagtatgtccagtttaatctggagttccaacggaattacgttatgatcttcgccaactttgagatggttgacttggatcgcgatgtggccggtgaccagttcctctggaccggaagagtctttcagctggagtctcaaggctcctggttttatcaggacggctggggagtttgcctggccgtggatatcggtgcggccaagctcactgatgacgggaaaccgactttctaggtgatgtatgtttgacggcgaactgatagcgaaactggttgtcgagttgaatgcggcgatgacatctgctcaagaggctttgcagttcccggattttgaagtcgtccagaaagctcagccgacccaacagggaacgtcaaccaggccgaccatctttttccagaaactgtttgacattcctcgcggctggcctgccaccgattggcacctggacaacacggcccgcaaatatgtagaaataactcgacagcatgtagagacgactttccagatcagttcccttcattggcagaatcctgaaataactcacgtggttacggcttctgatatcgccaactatgtgagggcttatttccaagctcgatccacgattgagcgcgtaaaggaactggacttcctcattcttcgcgtgtctcaaatttccaacgaagcattcgagaacgacaatcaccagttcgaattccacccaagttttgacatggttgtaacttacaaccaatatattcgcctgtacgaaaacgcagcatattcggccgatggggtattaataggcatatgagtctgaggcgcgattcagagctaatcgccgcgcacctccagatgttaagagccatgcgcggcaggtccgtttcagccggatggtattccacggctcgatatcctgacaaggcaggcggatcggtcggaatacaagtcgcgagaatcgcacgtctcaatgagtacggcggaactatcgaccatccgggcgggaccaggtatattagggacgccattgttcggggccggtttgttggcgttcggttcgtcagaaacgactttccgggagaaaccgaggtaactaagcctcacagaattaccatcccggctagaccgtttatgcgatatgcttggaacttgttttccgcagatcgcgccgcaatccagaaccggatagccatgaggctggccagaggacagatcactccagatcaagctcttgcccagatcggcctggcgttggaaggatacatagccagaagcatcaggaccgggccatgggtggctaactcagcatctacggtcaggagaaagggattcaacagaccgctggtcgatacggcgcacatgcttcaatcgattagcagcagagtaacataaccaggagatcatccagtgatcagtcagagccgttatatccggatcatttcgggcgtaggcgcaggcgctccggtcgcaggccgaaagctgattctgcgcgtcatgactaccaacaacgtcattccgcctggaatcgtcatcgagttcgacaacgccagcgcagtcttgtcatacttcggcgcgcagtcggaagagtatcagcgggctgcggcttatttcaagttcatcagtaaaagcgtgaattcgccgtccagcatcagcttcgctcgctgggtaaacaccgccatcgcgccgatggttgttggtgacaatctgccgaagaccatcgccgatttcgccggcttctcagcaggggttctgaccatcatggtcggcgcggcagaacagaacatcaccgccatcgatacgtccgccgcgacttctatggacaacgtggcgtcgatcatccagaccgaaatccgcaagaacgccgacccgcagctggcccaggctaccgttacctggaatcagaacaccaaccagttcaccttggtcggcgccaccatcggcaccggcgtcctggccgtggcgaaatctgccgatccccaggacatgtccactgccctcggctggtccacctccaacgtcgtcaacgtcgccggccaagctgccgatcttcccgacgcggccgttgccaagagcaccaatgtcagtaacaacttcggttcgttcctgtttgccggcgcgtcgctcgacaatgaccagatcaaggccgtgtcggcctggaacgctgctcagaacaaccagttcatctacacggtcgcaacttccctggcgaacctcggcactcttttcaccttggtgaatggcaacgccgggaccgccctgaacgtgctgtcggcgactgctgccaacgacttcgtggagcagtgccccagcgagattctggccgccaccaactacgatgagccgggtgcttcgcaaaactacatgtactaccaattccctggccgcaacatcaccgtttccgacgataccgttgcgaacaccgtcgacaagagccggggcaactacatcggcgtcacccaggccaatggccagcaactcgcgttctaccagcgcggcattctgtgcggcggtccgaccgatgctgtggacatgaacgtctatgccaacgaaatctggctgaagtcggctatcgctcaagcgctcctggacttgttcctgaacgtcaatgcggttccggcgagcagcactggcgaggcgatgaccctggcggtgctgcagccggttctggacaaggcgaccgccaacggcacgttcacctacggcaaggaaatcagcgccgtccagcagcagtacatcacccaagtcaccggtgatcgccgcgcctggcgtcaagtccaaaccctgggttactggatcaacatcaccttctccagctataccaacagcaacacaggcttgaccgagtggaaggccaattacacgctgatctattcgaagggcgatgccatccgcttcgtcgaaggatcggatgtgatgatctaatggtttgcggcggactcgatccgccgcgaccttccataaatggagtgaggaataaacaatgatcaacatttctgcgttcggctcgatctgccagttcacggcaagcagaactttcccgaacggattcaccgtcaccgagtttgccgacgatgcggaccccatcgacagcccgccgttcactgcggccgataccggcgtcggcctcaacggtgacatggtagtctggaaccgggcgaacatcctggaagtcgtcgtcaacgtcatcccgaacaccgagggcgagcgcaacctggcagtcctgctggatgccaaccgcaccgggaaagacaagtcgggcgctcgtgatgtcgtcggtttggtcgtggcgatgccggacggcagcaaaatcacctgcaccaacggcacccccatcgacggcgttctgatcaacgcggtggcaagcgtcggccgtctgaagaccaagccgtatcggttccgattcgaaaaagtgatcaaagccggtactagctgatgaagaaaattccgctgacagcagtccccaaccaggcgatctcattcaacgccggtagcagctattggaagattcgcctgtaccagaacatggacatgatgaatgccgatatcagccgcgacggcgtgatcgtttgccatggggtccgctgcttcggcgggattcctcttctccaatacagccatcagtaccgacctgactatggaaatttcgtcttcgaccgcgacgccgattggacattgttcggcgacggcatcaacctgttctatctggacggcgccgaattcgccgagtatcaggcgcttgccacgaggaaagaatgagcacatcaacgatcagaaccgggacgaacaacgatatccttttggacgacaatggaaacatggttatcctcagggatgtcgaagcgtgcgcccaggacgttcgggcggcgatgctcatgcgcaccggcgaaaacattttcgatgtgaactccggtgtgggatatttcgaatacatcttctcgccgcagaaaagctatgatgacgctcgcaaatccatcgcggatgcaattttatcctcaccggatgtgaccggcatcgagcagcttgacatcgacataaccggggaagtcttcggcgtcgatgcgaaagttatcaccatccacgggcctgttaccacaggagtttgaaatgagtaccatccgcatccaatacgccaacggcacccaactgttcctggacggcaaaaatccgccgcccctggacccgctgccctcgtttaacccgtctgtcgaagatctggaaggcctggaccgcgaaaagaacaccgacaagggcgactcctcgccggccggtcttcccgttcccccggtaaacgtcgattccaatgtcgacaacggcggaaccatcccggctccggtatcgaccgacgctcctgcggccgaatcggccccggaaggcgcccaggaagctcctgcagcgggccaaggcgacgagaaaggcgccgaggaagccccgactaccgccccagtagaaaaggccgaggaaacggcctcgccggccgctgaagaggaagccccggctcccgccaaggccacctctcgcaaaaccaccagcaagtaaggactcgacatgatcaacgtcagcggcttcggcacaggaattgtaatagtttccgcctcatcgttcccgatggggttttccttgtcgaagttcgctgatgatgagagtccgatatcctccaaagagctggagccgttcggatatgagatgctttatgacggcggcctattcgcctttgacaaggctgctcctctggaagtatctgtatccgtcatcgcagggagcgaggatgatattaaccttcgcacccttctcaattccaaaaagggatcattcagatttcttccgggaattatcccggacatgacgactctcgtggctactcttcctgatggcggccgcaccgttctgtccaatgggaccatcctcaagggtccggccatcgacaccatacagaacaccggacggcgcaaaggcaacacgtacactttcgttttcggtagctatcttggcgcccagactgcgcgtcaagccatttctaacgttatccaatcggtactggaggtggtctgatgttagggattttcaccagcctcctaagttcgcgatctttttcgattgtggaccaaaacacaaaccagctagttgctgcggatttgaggataagccgggtcaacacccggttttcttctgtagggcaacgccacatgctggaagatggtacgaccaagatggattccagaacgatccaccctatggaaatcatcgtcgaagtattttgcccttcaattgatgtcgtcgatcagatcaatcaattgctcctggatcgcgacacgctgtacaaagtcatcactcgcggcatggtattcgaacggatgatgtgtaccagcgaagcgctcaatcagactccggatatgatatcggcgactcctgcgcggctgacattctcccaagttctcgtccagaatcccaagcctataatgttcagaaatgcaggggactcttctatgatcgaccgagggctggccctagctgaagacgtggttggctcggccggcgatctgttcgactacgcagtaaacggcgtccagaacgccgcagacttgttctgaggtgccaattgaactctttcctcaagtctattctcaacacgcctactctcaccatacgcgatgatgtcaccaaacttcctgtctggaagagtcttcaagtcaagaaagtggaaatttactcgccggcttccgtagtgtcgaaacctctggcgacgaaagaccagacggaagctcaagtgtacaccgaagctctggacattgatgtgaagaatggaaagatcatccaaccggtgcgactccgcatcaatgctatctgtccggacctgtccaccgttgaaagtatcatgaacgctttcaatgataacacctcgactttcgctatcacttctaagtcgatattggccgataaaatggccatcatgacgctcgatgtagatcagtcgccagacatgttgaacgcggctgagatcaatatggaattcgagcaggttgagcctccagtattgaatgaattcgatcctgcgttccctcaagatagtccgacttatggggtacagattcaatctctttctgatgctaatttgctggatttgggcgccatcggcgattcgatatcttcggccgcaaaatcgctatataatcgcgtgaccagctacttctgaggatgtatcatgcttgaaatcaatcttcccgatggccgtcaaactcgcgtacaaatcgaggcgtggtcggcattggatggctgggaactccagcgccgtttcgtcgaattcgctgtcagccaagattccgacttccgccgctctttcaccatggaaatcctgggctatgcgaaagtgctgcttggcgacgatgacgccggcattccgctgaccaccgcggcagtcatcaacaaccacctcggccactggaagaacgtggaactggttttcaactctgttctcaagcacaatggcatcgacccggccacgcacgccgaccggccggactattgggaacaagtcggatcgcagatggccatcgcatttctggccgaggcgtccaagctcattggtccagcaatgaagatcgccgaaggactcgccaacaagccggagtgattcatgtctagtgatttggatgaattcatacttcggtatgaggccgatactgccagagccgagcgcaatctggagcgcctccagaaccagatcaggcgcgtgaacagcgcatcgacgagtggccttcaggatttgcgccacttcgcagacggcgcggccaccgaactcggcagagtcgttccgcagattgattctgtgacgagcgcaattcgcggaatgaacgcgcaactggcaataggcgccactggcgtggccctggtcgcggccggcgtcaaggcgttcatgaacaccagggaccagtacaatcagcagcgcatccaggcgatggacatcggcatcgccccggcacgcttggaagagtaccagagaaaattcgttcgccagtccggtggaaccatcagccgcgagcagggcgcggagatgaccaaaaatctggcagacactttccggcgtgcttatcgcgatatcgggcgagtcggcccggaagcgcgaattctgcgtatggccggcgtcgatgtcgggagcttccaaaagggcatgcggccgctcaacgacatcatcaccgatctggccacgaaaatggccaagctgaagccggacgaaatttctgcctacgctgatgcccttggcgtctcgcgagattacctgagcaccctggcgaagatcggcccggccatgggaaaagtcactgagatgacgactgcggaactccagtccagggtccagggcgagtccaacatccagaaattcaacgatgccctggcgaatctcaaccagacgttcactaccctggaaaaccgagtcggcgaaaagctcgcgcctgcgttcaccaagctgatcgaaatcatcgacaagatagtccaggctattcccaatgaagtggaaagcttcgcgaaggacaccaaatcccgttgggaagatggagtgctcgggaaggcgaccgttggcagtgatatcctatccctcctcagccccggcgccctgctcggtcgcctggcagcgtgggggactcgtcgcggcatggaagaatcagggctcatcgacaaggacaaggttcccggcgctcaaaccagcgaagacctggccaagaaacaagaagaccaggacaaggctactaagtccatgaaagagctagagaagctggctgaccagaccacgaagtctaccaatgattttgcggtggcgatcaacatgttcagtggcgcggtatcgtcgttcgcgaatgccgttgacgagcgccaagcttgggcagcctgggcgggggaaattggtcgggcggtcggcatgggaagcaccgcgccgacttcgcgggccaccggcgtctatccgcacgcgatctatgatcagtcgaagagtggcgcggccggacaagtcttcggcgagccaatcggcgcccagtctcttcgcaatcgcatgttctcgccgcagcgcaaggcagagccggtcaccgttccatcgtacatcaacgatatcatcaaagatgcttcgaagatgtacaacattcctgagctggacatcaagaaactcatatacaccgaaagccgattcaacgccagggccaccagcgaagccggagcgaaaggcctcatgcaactgatgccggaaatcgccaaggcgtatggtatcactgacgtatatgaccctcgccagaacatcctcggcggaacgcgcctactgcgtgaaaacctagatcgagcaaatggcgacatgcggttggccttgacatactatcatggcggactcgatccgaagaactggggcccaaggactcgcgcatatccgggtttggtaatgagcgctccaatcgagctgatggaagaggctcagcgcaagcagaaggctgcggccatgaccgtcgccaacgagacgttcgccccggaaggcggcgacatggacattcgtccctatgatggcgggcgactggaaaccccagaccagggcaagaaggaagatgagcgccgcgaagcccgtcgatatgacgacagggttgtgcgaccggaaattcgcatcatcgaccgcatgccagaccgcagcgacggcgaaatcctcaagatgtctaggcgtcaagaggccgaccgggcagactctggattccggaagttcccgaatcaggttcgcggcgagaccaagcagaacatccaggcccaactcactgccggagccatcgcgcaagtgatcggcgttaaccccaaccagatcatgcgccgcgaaatcagccgttctgacttgctattcggatacaaccaagccatcctgggcaagcagcaggagatcaaggccgccgcgaccgaagccaacaacgtattcctgtctccagccaagctcgccgaagccactgccaaggtgaacgccgcatcgcgagaaatggatattctcaggacgtacggcgagcaacttctgaagagcgctccagagcgcggccaggagctgaccatcggtcgaatcgatatgttggtcaacgttaccggcgcgaattctccggaagaggctcgcgagatattcagcaagcagactgcagaccagctgactacggcaatccaagatgcccaaaacgattccgcaactaagatactctactgatgaaaaagagaattctgcgagtaacattcaacatgccttatggacccgaagtcatccgagaggatttggatgttcgggtccggatcatgaaggctgcgttgcggattcagaaccgggcgactatggaaattttcggcctaaccactcagctgcgtgagtctcttctgtcacagttcaccgcgtggaagcaccggcaacgtcaagtcggcagggaagacgaattgatgatcaaagtgtcggtggaagccggctactctgaccaggggcgcgagcaagtttccagagtctttgtcggcgaagtggccattgtcgatatcatttcgccgccaccggacattggaatccgcatccagtgctacaccaggcaaatcgacaggacgaagaccattcgaaatatgccgcccgccaacacgacgttcgtcaagttcgtcgagtggggcgcgaacgaaatgggattgaacttcatctgcgataccagttacaacgatcaagttctgaagaatccgggccggtctatcactgtcgcgtcggcaatcctggcgtcgattcaggatatgtacatgccggacgtggccgcgttcgttgatgatgacatcttgatcgtgaaggatcgcgataaagtcattcgccctgatgaggtgaccaacgtcaattcgttcgttggaattccatcttggtctgaatggggcgttgaattccagtgtctgtttgagccatcgattcgcgtggctggaggcgtggcggtcgaatcgctcatgaacccgagcgtcaatggcaactatgtaatcactgctctggaatacgatttggccagtcgggatcggccgttctatatcaaagtcatggggagcccagcagcataatggccagggaaatcaaaccgttcaacatgttcggcgttcactatacttcgcggcaattctctgctgccgatgggctcagcatgatgtcggaaattcagagtgtgccgccagaagaactgctcaagggaaccagtgtattggcgcatccggaagatcatccggaaggcatctggcttcccttgaccgctgcgaacatcaatctttatgtagttgatcgggcgaaagtaatagctcccgtacaagttcttgcacttctgtccgaagtggtaattgactggaactttggtttcctcaaagactggaccggagtcaagattccatccagatttgtcgaagatatcaaaagcgtgaagacggcacattcgccttctgtagttgcgagtttggtagcgaacggctcagcctctatgcgcgagttggaagagtattattcgactcaagatgcatttaagatgatagacatcatgacggcgaagagcgtgaacgaggccttggcgtcagaggcatcgcacaacagaatcaaaaagggataattcctaaccgggcctgggaaggctatactagacctgccaaatcagaggctttcccatgtccaatatttctctaacatcagcaaaagctcccgacagaacgcgactgatcgccgctcttgacgctcggtcgcgacgggatgctctagatttcgaagtaatgataccggctcaggttgttcaatatgaccggaccgagaatatcgcgacgattcaacctctcatcacctgggttgacacggaacacaatgccgtccaaaggcaccagctggtggatattcctgtaatttccatgggggctggcggcttccacataagtttcccgattcagcaaggtgacataggctggatttatgcggccgaccgcgacacgtctcagttcctggagtcgttgtcaatgtcgaagccgaacaccggccgcatccataagttcgagcacggcctgttcatcccggacgtattccgtcgatacaccatcaattccgaagattcggccgctatggtcatacaatcgacttctggagcgaccagaatttcgattcgcggcgataacatcaagataactgcgccgtcgaacgttactgtggatactccgcaggcgaacttcaccggcaatgtgactatcgctaacaccctggtcgtaaacggcatcaacgtgaacaaccacggccacctcgaaaacaacccgcctgatgcccggacgaaaggcggcatggttgcttaaggagattttcatggcaagttttgatttttctgatttaacagcggggggggttgtaatggctaactacaactacatcgtcgatactggtgtgatagtcgcagataccgccgacgttctgagcgacgttgaggccgagttccgcgccgccctcggtgccaatatcaacttggccgcgagcactccgcagggatcgcttgtcgcggccgaggccatcgcccgttccagtgtcatgcggaatgaagcgcgaattgccaataccataaacccgaacgtttcattcggaacgttcctggacgcgatctgtgccttgatggggatcgagcgcggttctgatctgtcaaccttcggctatggagttcaagtcaccggccgcagccaaacccgcatttccaccgggtctagggtccagactccggccggcgcgatcttcacagtgatgagcgatgtcacgatccctgctggtggtgtcgcgaccatcgatatcaaatcgcaggagtatggcaacattcctctgccggtcgggaatctcatcatcatcgacggaacaattggatggtccggagcaaaagtcatcgcctccactcgcgtcgatccgggcagccgccaaatgagcgatgcagagttgaagaatgcccgcgttaatcgattggccatccaaggtcgcaactcgactatggccatcaaggcgtatgtgagcgccgttccaaacgtcacgtcggtgaacgtaatcgagaacaacaccggcgccgttcaagtggtgaatggagtctcgtttacccttccgtatgctgtttgggtctgcgtcgccggaaacccggataaacaagcagtcgccgatgcgttgtgggccgcccataacggcggaactccatgggactatggtgcgacgaacaacggcgtcccggtggacggtccgaatggcgttcctgtgcgcgatccggcgtccggtcgaaagtatgtcgtgaagtggaccactccaattatgtacgatggatatgttaacgtaacagtccagcaaggttcctcctctgtagctccggaagccattcagaacgcagtggtcaattacgcccaggggaaagtggaaggggaagaggggctggtggtgggcgcgagcctgtcggcattcgaagtcgccggcgccatcgctcgcgaaattcccggcatctacatcaaactctgccaggtcgcgtgcgtcgcggctggctcgccggctccggctccgggcgatttcacttcggaatacgtcatgagcgcattcggccaggctaccatttccgttggtaacgttcgggtgactttcgtatgactctgcctgcgtacaattcggacatccaacaagctctgaaatggctccataaccaggcgcctggaatcaccggcctgatccagagaaaggcgcaatggtatgacagattcagccgccaattctgggccaactgggagcgcgacgttttccacttgaaaactgccaacccgttcggccttatggtttggtgcatcatcctcggcacgccttcgaaagggttcggcctttatccaaaaaacagttcttgggcattcggtcggctacgccagaacttcatctatagcggtacacaagttccgccaccggcagacgcatcgcctggcggcaacttctacggtggcggcaatgccgaaattctcaacttggacgaaatcaggaaagtgcttcagctaagatatgtagcgctgatttctaacggctcgattgcatatgtcaatcgcatgcttcgttacatattcaatgatgatgagccatgggacgaggcgaccggtctgtacttctatctcatggactcaaccggcgagaatggccctgtggagaacttggcgatatatcggaaagattgggaaggtatggtgctgttgtccagttcgcccagagcgaaccacgtgctgacatcgacccctgccagcgacgccgattggccgggagtcgatccggccgcgagcggtattccggtaacggtcgaaacggcgtccgctacggccccggacggctccgctacggtgtgcaagcttactaagccggccgggagtaccgcttacgtctccgcgccgatagatgggccgctggggtccggtagcactgtaacgttctcgttcttcgcgaaagccggctccacccgtttcattgcaattcagtcggctgccgatttccccagtcgagccgatgccgttttcgacctggattccgggaacgtaatcagcgatcagatgctggacagcagcgtggtaagcgcccgaatgattcgtctggaaaatggctggtggcgttgcgttctcacgaccaagaccgtcagctcttcgttccgcgctgcttacgtcgctccggcagaaaccaacttcagctggattgattcgaattccagcgcggagattgacgtgcttatctggggcgctcagatcgaactgggtgatactccaactggatacttggaaactaccggggcgcccgtaaccatgaccgattatgttttgcagaacgcccagaccggaacagtcaagttcacacagccccttccgaccggagtagaagcgtattggactggagactggaaaggcgggactgcagcagaaccggccagattcgcagtcgggaatgggactcaggatacgtttactctgtcagaccccgcatacatcggcctacccaccagtggggcgttcaagctagaatacagggttggtccggcgcttaatctgtcgccgcaattgatcaacctcatgaatgaccgggcagttggtattatgccgacttgcgccggttgcgatgtcaaagtcattcaggagtaatgacgtgatcacacccgaactgatccccagtccgtttgccgctcagggcgataaagacccgattccacaaacctcctccactggcttcgcaaaccttcgcgacggctatacgccggactacgaaatcagcctggcgtcgaacaacccgcaggccaaagcggtcgagcggaagattcaaaaccaactcttcttcatcgctacccagaacgcacaggcgtggcagcgacaaatggcgccgccgtggtttcagggcatgcctggcggctacgaacagaatgcagaagtcgtgcgcgtcggaaatgacggcataatgcggcgctatcgttccatggtgaatgccaatgcgagcgaccctctcagcagcacgacttgggaagaacaacccgcatggtcggcgatgcgctccaacatcccgatgccggccggaggcccaggcctatcttctggcggagaagtcatcacgaccggccgcaacttcaacgacctgttaaatgggacgtgggagttcttctctgattcagtggttatcgcttctcagaatgcccccgtatatccggcttccgctggtgccgctgctggcatgttggaggcgaaatcttgggtgtccggagccaatacgttctgcgtccaacgctacactgatcgcgtcgggaacgtcgctgtgcgcgggcttaatgccggagcgtggaccaactggatgtatgcggtaaacgtcatggcccttcaacaaggtcgggtcacctatggagtcgcggccggatcggcgaacgcttacacgttgacgctagttccgcagctccaaggcggcctggtggatggcatgattcttcgggtcaagttcaacaccatgaacaccggcgcctccaccatcaacgtctccggactcggcgccaaagccatcgtcggcgcggccaacttccctctcaccggcggcgaacttggccaaggactcatcgctgagcttgtattcgacgcagcaggcgaccgctggagaatcctcgcaggcgcgccacgcatccaagttggcaacgccgatcaagattaccaggcccccagctggaaacaggtgaaggattacgtcgcgtcccaaaagctgactgaagtggactgggctgacgtcgtcaacaagccgaacgtcgctatccaagacactacgccgtggttcgccaatctggagttgtctgacgctcggcctttcatcgatttccacttcaacaacaaccgcgccaaagacttcgactatcgctttatctctgaagctgatgggtcgatggcgttctactctcgccaggggtcagctggtcctacccaggatatcctgttcagcaggtcgaatgttacattcctccagccgcgactggatgttgcgaaaaacctcgcgtacatcgcgaactctggccccctttggcagaacaccactgccgatcagcctggttggaaattcaccttcgcacaaggtgtggacgccaacaacaacgcggttatcgcagtcaataccaccaatccggacggctcctatcgctcacagatcatgcgatgggactgggcgtccacgaacgtcatattcaacaatcgtccgctctttgccggtcaatacactccttgggattctggaaactttgatccctccaccaagttgactgtgaatgccacgaaccaaatcgccggcccgaccgggattcggaatacaaacggcaacaccggcaacatgaacacatggggctccagttccacgacggcttcatacggaaacgctgctcttcaaatcttcgggaaaggtggtggcgagcctgccgcgctttatttcgacaactcccagacaggatggtatctggggatggacaaggatggacagctcaagcgggccggctggtcgctcggcaataacgcctacgtgattaccgacgaatccaacattcgtttccacgtgaattccatggctggcactcctgtttggggcggaaacgaattctgggggccgtggaactttaatccgaacaccaagctgaccatcaaagccggcacccaggaaactagcagcactgcgatattcagcggaactatgccattcgctccaatcgcatcgctgtccgattattctcaggcccctttgacggtttacaacgcgccaaccggtccgtctgcaaaaccggccgtcatcgcgttcattcgtcctggcaactggggcgcgttcttcggcatcgataccgacaacaagctgaaatggggcggcggatcgctcggcaacagttccagggaaatcgccgattccagcaacatcatgaatctttgggcggccaacccgaccgcgccgtcctggaacggccaaaccgtttggcgatccggaaactttgatccggcgacgaaagtggatttgaacgccgcgaacgccaccaacggcaacatggtcttcaaccgcatttctggaactggtagcggcatagcttcgtcaggtcgagttggcgccatcaacctgcagaatggggcgcattcggggcaagcggcagcagtcactttcgagcggggcggcagtatcttcgtcaacttcggcttggataccgacaacgttctcaaagtaggtggcggaaacctgggggcaaatgcctatccggtcatccacgccgggaactacaataactacatcaaccaggcgctggttcaggtgggtcttggcggagtcggttcctatggcatctttgcggttctggacaatgccgctccaaccgcgaccgttcgaccgggagtggttgtggacggttccattctcatctactcgtcttgcgccgcaaactacaatagcggtcaaaggcctgccggaacttggcgctgcatgggatatgtagtcaaccgggatgccaacactcctgactccgcgacccttttccagcgagtgacgtaaaatgaaatggacgcggatcagaaaccctcgttggctggatgaaacaaacatccacgccatggtgactttcgaggggatcggggaagttcctttcactgccaatccgcacgacgtggaggcccatggaagggccatccacgctgcgatcctgtccggggagcacggacctatcgccccggtagattcgacgcgggagaaggccttgcaggacgctatacgagcaagggaaaagcgggctatccttcgcgatacccgctggcccatagatcgtcacgacgagcagaggcggctgggtatcgaaaccactgacggacctgggctgatagcagctcttgtttcctggaggcagcagattcgcgattggaatagcggggatcggccgcgacttcccatggctctgaaaacgatgtttaaaaatcaggagtattgatgaaaatcacgaaggatattctgatcaccggaaccggttgcaccacggatcgggcgatcaagtggctggatgatgtccaggcggctatggacaagttccacatcgagtcgccgcgagccattgcggcctacctagccaacatcggcgtcgagtccggtggactggtgagtctggtggagaatctcaactacagcgcccaaggattggccaacacatggccgcgccggtacgcagtagacccgcgagtccgcccgtatgtcccgaatgctctggcgaatcgtctggcccgaaacccggttgccatcgccaacaacgtgtacgcggatcgcatgggcaatggatgcgagcaggatggcgatggttggaagtatcgcgggcgcggcttaatccagctgaccgggaagtcgaactattctctgttcgccgaagactccggcatggacgttctggagaagccggagcttctggaaactccggccggcgcgtcgatgtcttcggcctggttcttctggcgcaatcgctgcatccccatggcggaatccaacaacttctccatggtcgtgaaaaccatcaacggcgccgcgccgaacgatgcgaaccatggccagctccggatcaaccgttatatgaagaccatcgccgcgatcaatcaaggctcctgatattcgcccaaaagaaaaggccgcttattcagcggcctttttgctttccggctttgcctcttcaaccatcttgacttcaaccggcgcggcggactcttcctgagtgaccgaatccacatagttccctagtgaactcagaacgccgattaacagcgctcttactaccttgtccttgactgtctcgcctatgatcttggtcagaacggatattaactcttcccggagtctggggctgattcttggccgaaagcgcttgcgatgctctttgcgtttcatgtttagtcctctgtctgcggtcttctcctcaccccgataatggcttggggatgcgctgtgttaatcggaaggttcgggcgctattataactcgtcgaaaatgctcgcgcttaactgtttaacgatacgcaccgcgatattaaatcgccttctttctggccaaggaactctggcggccgggtccggtctaaggcctaatttgtcgacattaaaacgagaaaacccggatcgcctgtagggcaatgcgtccgggtttatttctatctagtgtacgctaggatcagtgacttccgccccacccgtccagccagcaatcgaagacggcgtgcctcggcttgtccttggcgccatgggagaagtgcttgaaccggatgacctggcccttgagatactccctgtcattccagcggcgctgtttctcgtcgtgggtcaggctggaggccgacacgttgaaagtgactccaggccacagacgttcgttgcggcagactaacgcaccgaccattccggacggcgacaggttttctgcgtggctggagcgggccgtgtggccaagttcattgatgaaagcttcattgttgttgtgcatcaactcttccacgtcgatgatctcagcttcatcgtagtcgtatcgcttgactttcacacagtgcccttccttggcagtcgagcgaccgaacttgtacagcccatcagcgcgcttgcccatggaaccttcgaatcccagcattgtgtggcggcgctcgacttcgctgaactgttcgatggaagtcaccagttcctgctcgaccaggtgaatcctctcatagccgaggcagtgacgaaggaagttcacgcgctcggccgccctggctaggcgatcttcggtcggtgcgcgaggatcggtgaaatcatcgaacacatggaaagaccagtccggctcaccgctgtggcggcgaagatcgccggacgacttctggaaaactttcgggtcgctgatgtcgccgcagaccagctcgccgtccaggccgttgaacagtttatcgctgaggtattcgtagatcgattgattgttctgccgcttgagttgacgagtcagcgcctcgccttcgaatacgaagcagcgaaatccatcgatcttcggcgagaaatacatcggcagttggccttccagcagcttcgggtcaaaattcgatgcgagcatgggtttcatacagttctccagaaaagaagcccggcgaaccgggctaaatggcggtaagccggctcagatggtttcgttggcgtggttcagttcggccatgatcgacgcatagcgctcgtccgactccttgatgaacacgccgtcgtacatgacgcctttgcgatccttgatggtgtcgtaggccgcagcgtagcaggcgagcatggtagtgtcgtgctcttccgtcacatcgaacagagtcatgacggccaataccaggctcttgatggctaatccatgatttccgcgtgccagcgcgccggccagatgtccgaggtgctgcaaatcgtcgccgtaggtgggatgaacttcgacagtctcgctgaagaccgatagatgatcgaacagattttcaccgagttgcgcggccatgatcgtggcaaccaccattacgtcgccgatgccgtctttcacctcgactggtttgttataaacccaggcttcgcaaacttctgcgaactcttcgaccaacttcaggaattgatccttggccgaagagcctttgatcaggttccggtcggaaccccatttaacaaccaggtcgtgaagttcgcgactcatgatttgttcgatattcattctttcgattccttttggatttgggattttactgcgttgattatggacgccgtgctctggcgcgatccatccttggtggtgccgaagtagaaggccattaccgacttcagctcggcgaaccaatagccgatgatggtgccgatggcgaccgaggatgttgggtccatcagagcctcgcggccgaatgtgaagattgcgatgatgatgagaatggagcctgtcagaagagcgaatgtaatcgccgggcgaacgaaatcgttctgctgcgccgcaagcctcctcgcagaatccctgtcggccgcctctgcggcgaactggctaagctcggcctggagctggttctgctcagactgaaggcgattttgttcggcttgaatcgcaagctcctggagacgaacgcgctcggcgctctggagttcggcgagacgcgctagagcttccggattcgcgtctagagcgctagcgaccgaggctgggtcggccttcgaccccagcgccgtcgcgacgatagcacccacggcggcgcctgcaggcccacccaggagcgaccccagagccggggcagccgcgccgatcttactacctatgtctttccagtccatttcgactcctcaaaagaaaggcgccattacagcgcctttctccggccggtgattcagaactcttcggcttcggtcgcatcgccgacgccaccggcgtcactgcgaggctgttcctgcttgctgtagtccaccttcacctcgccgccgacgaacgacttgtacaggtcggccgcagccttgaagtgatccgggttcttcaccaggccttccagctcgaactggacgccggaccagctacccttgtcgttcgacatgcctacggtggtcatgcggaccaggttggcgaaagtcggcggggtgcgcaggccctgcggggtctgaacctttttctgggacagcgcggtcatgagcttcttcgaggccttgatctgcgaagacgacagggagatcagagcctggccgaaatcgccggtgtccggatcgatgacgatgacgtaatgaccacgggtgtcggcgaagtagtcagacttcttgtcgctgaccgaaccgtcttcgttcggcgcgtacaggcgtccttcgacttccttcaccttggtcgggtctttcatcatttccttgaagtcttcgacgctgatggcgcccttgaaaccgccctcggcctcgcgaccggcccagcggatgaactcgcggcggtacgcggccggaatgatcagcaggccggtcttgccgtcgtaaatcttgccggtgacggtgttcaggaacatgccggccttcgcgccttcgatgtacttcgggtcatcttcatcgacctggggagacatcttctgcagaacctggatgaaggggatggcataggactcggcatcggcgccttcgaagcctgcgccgtcatacgatcccaaatccatgaagtcgggaacttcggtagttgcgacggcgccgccaccggtggcaactgctacggccttggtttcttcggttgcttcggaagtttcggttttcttaccagccatgttaggctccttgtttgtcgaatttcagttatcgctaactgtgggtttataataacggaagttgcaactaagtaaagcaaattacatattaagatttgctctttttcatcttcggtttcgtgatcttggcctctttgtattcgtgcacgccgatgaaatctggcagctcttcgcccttctccagatactcgcggccgaacgcctggagggtctggtaatggacatcgcggttgatggtggcgtcatagccggcttcgatgatggcctcggccgccttcttcgcatcttccatttcgccgcgaccgaactcggccagaaccttggtcttgatgatgccgtcgttgtctgtgtcttccagccacttccagaacttcgatttgttctcttccttgacggaaatgatggccttcggctcgactttgaccgtgcgaccgtccgccagagtagtggtcttttgaccaagctcctccaacagctccggaatggtgttgcgcttgagggttttcagctcctcttccttttcggccagcgccttttgaagatcgaggatttcgacgtccagctgcgaagccttgtccaccaggttcagcagtcgatggccgatgtcggtagcttcgaccgccatttcatccatgacgccaaaatagtcgatttcgcccggtgcgttctctttcaggtattccgggatttcaagctcttgctctttcatgtccgcctccaacttagtgatgttcccttacttgaactaagtattgagtagatattatgccgcatcttccttgatacggctactgatttacatattaaatttcgtcgcgagtgctaacgtcagcctcaaacactccatcaacgacataacttgccaggttgcgcttccactccaagctgacctggattttctcgtcgatggaatccagacagatcaggtcgaagtacaaaaccgagttgacggtgccgattcggtgatttcggtcttcggactgcatccgcaactcgttgtcttcgtcggtcgtgtagtaaatggccacgtctgcggcggtgagcgtgatcccgatccctgctgcggccgggttgcccaggaacacctggacgcgcttggcctggaaatcgtcgatcaatttttcccgctcggcctctttggtctcgccgtagtatgtgccaaacgaaattccttgggcctccagatacgccttgatctggtcgatttcctgaatgcgcatggcccagatgatgatggagcgctccgggtcttcctccagcagaccctccagaagatcagtaaacacggcgaatcgcgggttgtcttcgggcggcaggatcaccggctcgccatagacgttgatatagccggatgcaacttgcttgagcttcgaacgcgctgccgcagcatcgaaagatacgtccagcatgaagtcttcgttcttgagaacgaagtggtagtcttcttccacgcgctggtaaacttttcgttgctccggcgacatttcgaagtatatgcgcttgtacaccttgtccggcaggaatgggagtgcttccttcttcgtgacccggaaactgtgcggctcgatcagggaccgcagcttgtccaggttccggaataccgggcgcccacgctcgtctttttcgaccagttgcggcggcatggtttgcttcccttccagcttgcgcatgatagcgatcattcgaggatcgtcactgggaaccaaaacggaaaattcggccacgaacgcccgataggatttcgttccaagaattccatcgcgcaagaattggaactgcatgaacaaatcggtaggcgcccgcgttagaggagttccggaaaggatgcgacgggccacggccttctcacccagctttacgatctttttcgcacgcttggcttgcgggtttttgatcctcgtggactcgtcaacaattgcgcaaaccttgaaagtttcaaggaatcgcaagacttcatcatagccagcctggtgattgatggcgtcaacgttgatggcgaagacgcgaagaactttttcatctgcgaacgtctcggcatacagacgatccaagcgcgccctggcctttttggaagtcggtcggccgcgccaatccacgctcaaagtcttgatcgcgacgtgggtgggaatctcgcgcagaatccagttggtgtggacgcccttgggggcgacgatgagcagcgcgtccacgcgcccctgtaggaagaggcgaacggcatcggccaaagtcgtccaagtcttcccggtgccctgctccatcaggtatgcgaaattccgtttgttcagggacgcggccagggcattgaactggtgctgcatggcctcggtcttcatgcccttgacgggatatgttttggctttcatttgttctccaaatcggcgaggaactgaacaatgttatccagtccttctgcatagctcgcaatttccaccaagtcgcggctgttaagctcgaacaaatcgagcatgggattcagcagcagccaatcggtcccgatcttggccaggacgaagccccggccgccccagccgatccgttcccgaaggaaagggatttgcccaggctcgaaacatctggccattgggcaggtggaggcgcgtttcggccactcctccaacgccttgaactcgacccagaactggacacctcgacgattcaggcaaatcgaatcggacataccagaccgccgcgtctccagaaagtcgatcaggattcggccgagtgagcgttgcttaaacgcattcgccgctttcgtttcgcgatcattcatcgccttcgccctctttggaaactttctctgcttgcgctgccagctttgctttttcgcgttcggtcaaaatcctcttgacggccttcacgatgaacatatcgattccgctgagcttccagcctttgatgaggaaccaagagcctgtcggcgttccttcggcaatctgctttccgtattgcagatatttttcaggcctaattctgaaacgtatcggctgatcaaccgagtcgtcaacacacatgatgtcgaggaactgcgactggcccttgtacaccgggtttttcccttgatctgccctcttcttctggcgaatcggttcgttctcatcagacagaacttttttcaccatcttgacaatgaccaggccatcgtctccatcgcggatatcccgaatgttctggatggggtttccggaagttactccaaccaactccggattgtcataagcatgaccccagagcgtgtgagcttcgttcaaatctgcgaattgaacttcagaattcgacagactcgcggcgactttctcccaatcctgaagcgtcttcaggtgggttccggccagctctttatattgagctttcagctccctaagatcggcctttaactccttcagatcggccttcaacagtttttccagttctttgtcttggctgattttcgccgaaagaatctgggcttccagcgctgctacatcagcagccttgtcctcaacatcctgtgccgatatcgggcaattggccagggcgatcctcgcggccttgacgtcatcacgaaggcgcaggaacttctctgccttcgccgggccgaagcctttggcgttcatgatgccgccgatcaggcgcccgtccgccactacccagttaagttcggaatgctccgggtccagggccgtatattctacgccttccttggccaattcgcgaaggatggacacagtttgctggtcgtctttcgccgcccgaagacacgctgccgcgtattccaggcgatgataccgcttcatgtagcaggtccagtacgtcaccacagcgtagctgaccgagtgggagcggttgaatccccaggcgccgaatgtcaccatttcctgccacacacggtgagcatcgtccggggcgacgcctatggtcttggcaccctcgatgaacaattcccggcgcttgttgaagaactcttcaccctttcgcgcagacatggctttccggattgccgatgtctgctcccagtcgaactgtccgatgtccttgacgatggacatgatctgttcctgatataggaagacgccgtacgtccctgacagatattgctcgacctgcggaatggtataggtcacaggctcgcggccggccacgcgctcgatatacttggtggccataccagaagacaacggacccggacgggctagcgccgtgatgtggtcgatgttttcgaacgcggtgatattgatcgcgtttgcgaccgaacgaactgcctgcccttcgaactggaaaatgccggacattttgtcttcgttgagaacatccaggaccgccttgtcgttcaaaggcaagtcgtacaactcctgcgcagtcacacagttcgcgtcctggatgacgcccagagttcgaaggccgagcgcatcgatcttgaggagattcaaatattccgaatccggcttatcgagctgcgcgacgccttcagaagttaccgtacagaagtcgataacttcatcgttgcagaccaggatgcctgccgcgtgtacgccagagtgggatgggtgaatttcgaggtcgcccatgcaggcggacgcgatctcatacttttcgcggaagtcgcggccgggctgagttttctcgaaagtgtcctccaatccttttccatatcgttcgtccgccgacgtatattcaatgatcgagtttttgatgttgtctgtatcgtggaaggggatgccgaagcgctttccgacgtgagcgataaccgacgcggccttgagcgtgttgatgttgcccaacttcaccacgttccaagtgccatacttctgctggagatattcgaacactagatagcgatgggtatcggcgaagtcgatatctatatcgggaagatcggaccgggaaatgtcgataaagcgctggaagagaaggcgatgagggagcgggtcaacctcggtaatgccaaggagatagcagaccaaagagccggccgaagagccgcgagctgggccgaccagcatatgcttcttggcgaacgcgaccagatctgccacgaccaggaagtagctgtcgaaatctttcagctgaatctgcttgatctcttcttggaatcgatcctcatactcctgggtccattccttgatgtggccgcgactgagacggtaggcttgcccctcgcgagccagcgcgacgatatccccatccaggtggatcataggcgccttcgccagtttcacatccgccaattgttcgactaccgcacgagtattggcggcggcggcatcgaactcttcgcgagtcatgatgtggcgtaaacggctccacaactcctcttcggtggcgatgtggcgaaggccgaccgattcccgcaccttccaggccgacgcgaaatcagcatggtcgatggacggcatgtcgttgtaagaggtaattaccaccggctttccgaatgccctggccgtctccatagcgccgtgcgcggcgaccatcgacgcgggattgatgtcaatgtagtcgattccggccagatccagatgggcataggcctcgccagcgaatttgatgacgccgtcagcttcctggaattcttgcggagtcaatccttgattctggaccgacttggaagtcaagcggtagaacttctttgtgtccttggcgagcacccaggctttgagcttcagctctttctcgccatcgtcggcgcatttgatcgggatttccatgccgaatccgcgaggcagttctgccttggtagcggcttgctcccaacggacgtggccccatgttccatcatcgacgatggcgacgaatggggattcgatctctttggcgcgctcgatgatctccgggaatcgaccatatgcggcgccgtaggagtaaccagagcgaacgcggagctgagggaaagacatcatgcggcctccattgcttgatacgcccgatacactcccatgcgcttgcagacttcgtggagcagccgcacgtcgtcgagcgcccggtgtttctgtacatatgggccgcagtagtgctcataaagatgttgcagccgcatgcggtggccgaacaacggcgccgactcttccacagtacagatatcgagcgatgggaaattgacttcgtccaggccgaacttgccgcgagccagatcgcaggtcagcatgaacttatcgaatggcaggttgtgggcaatattcgcgtcggctctggaaaagaagtcgcgaactttctggcgctgatcgaggaaagatgggtgcttgaccaggtcttcgttcttcaacccggtgatcttggtgatgatttcctcgataacgattccaggattgcagatgaactctacttcatccaaaatcttttcgccatcagttatcacgccggcgaattcgatgattctcggctgctttctcagactcaccctttggtggaacgggagtcctgtggtctcagtatcccatacagcgaatatcatgtttgttccctcttatgtcgaaaggccggctgctttcgcgaccggcctgaggagtataccgcgacggctgaagatttacgccttctgtccgtctttcggcgtgatgcggccggagcgcatggtggcgtgaacaaacgccgaatagttgatcgagtccaaggccgaatcggcatccttgaacccgctattcgccaggcgagtgagtttacccaccatgtgcatcacgaacagggcgagtcgatgatcatcggcggtcttcgccaccaggccgttcgggaagaggatttccatgatctttccgtacatcagatcattgcgaccataggtgctctggcgggcgcggaagacttctgctgcggcgtacagattgttgagaacatcttccgcgaaatcatctggatgggaatcatcttcgcccggccagacggattccatggcgaacggcgcggcgtcttcggtcgggtctttggactgagccgtttcagcgagcggagacggggctttcgcaaacgcctcgtcgagggtaggggcggagttcggggcggccgggaacggctcgcctgccatgtcgttgggcgcgctatcggggtcgctatcggccgcgacggcgaagaacggcgcatcgcaaccttccaggttcaggatgtaggagtcgatccccaggctattgtaggcatcgatgatgtcctggcgatcatcgaacgccgcgacgatcttggtgacgccttcgattttcttcaggatgtcgagcgcgactgcccgcttgaactgaggcgccggctcggtgttaccatacggccgcatgatgagttcatactcgcgatgttcggcgatgccgagttcgcggtggagcttggccctggtctggaaaaagtggttgtcggttcggccggtgacgaagaaaatcatgaggtcggcgtcgatggcattcctgatgcgacctacagcgtgcgggttgagagtgtccttgtcgagacgagaatgatactcgtcccattgccgttccaaggcgaagctcttgcggtggctatcgtcgaatacgcagccgtccagatcgaagatgatgatgccattcttgggtttgcgttccatattcagatttccttgatggttgctttctgggtgacggttttatcgatgaacgtcccatcagaagtgaggcggaaaacttcgccattctggctgaaatcgaacgattgcacgttcatcgtgatgcgaatggattcgctgccgttggtcacttcgacttgtgcgatgtcgccaactttctcgacaatgatcttcatgttacatggacttcccattaacggccacagggctggcctcttgtttttccgatccccagaaatcgcggcgcagttcttcctgctcggccgagcgatccatccaggggcgatagaacttgcactggtaaatcggctctaccggcaaattaaccacaggaatctgaccgggcttcgtctccagagcggacatgaagcggtcataggatttctgctggatttcctcttcattcatcaccttcgatccataacgcgggaaggcgcaggagccggtggcgacgcagtgcggctggagcaggctgtcgaacatcggatagacttccagcaccagccggcgcatttcgcggaacgcttcctgatattcaccctgggtccgaacgcacaggcgaaccttcgccatgtcgctgagagtccgcaggttgaacttggccgcgatcttcgtttccatgttggaagggatgatggcacgagcatcctgaagcgatgcgccggcctccaagagcttctggtaactggtctgcgcgtcggcgatggcatcgtgccacaggcggttcagctcttctcgggcgtgataggtcgggtccggctcgccattggccgtcgccggctcatcgaaatcccagcggaatgcttccggttgaactacggcgctcacctccagggcgcgactggtttcctgctggtaagccccggtgcgagtccgaacgagttgatgggtgaaattcttgctgacgccctcgatctggaaaatgaagtccacgaactcgaacggcgagcggatggtgtccagcatgtacttccagtggtcgagcttttcggcctcggtcatggtcgccgggtcttggccgcgcatgcgggtggacttcgtgcccaagagcaattcccaggcgttctgggtgtaactaatcagagaaatcttcatcagaaatcttccggaattggcgtgaagtggaatttttcggtgagagccagggccaggccttgggcttgctccctgttaaggccatactgctctatctcttccttgagcagttcgcaggcctccaggcgcgcctcatactcttcagcctcatggacggatgagaagacggctccatcagaggttcggtacacaagttcgatcgccatggtgacctcagtagcagcggatgatttcggcgcggatgtcgcgacggtcgaggtagtgctcacgaatcttgtcgcgggcacgctcggcctcttcgcggctgccgaacgacaggttgaacgacgtgaaaggcttatcatccagttcagcgccggtctgatacaggattcctaccagaacgaaagacggcgcggtcttcggctgctggtcggtttgcagttcgagaaaggacatagggagacctcttcaggatgatctggtgcgtacattaatagcgctcctgctgagcagctacggtttccggctcatagatgagcatgtcaacgatttccgggcactggcctttcacccagtcgatggccgcgaccagtgcggtggaagcggagaaagagaaagtgcggtaagactcatggatgtacggcgaccccatggaatcgcgctcggtggtgcgcgaaatgactgctttgatattaacgatccggcccatcttcggcctccactttagcgattatatcggacaggcttaacttctcgccattcaggaaataactggcgcgaaacttcctgtcgccctctggcccgacgattcgggccgttattgtgagactcccgccgccaagggcatcggtgtcccggaagaaagctagcagcgcccgcttgagcgctgcttcgcgaggatcgacggccattaacctaccacgttccagccgtgctgagcgcaccacacggcaccggtccctgcaggaagactattcagaaggaacacaggagtcaggcggccatcctcggtcatatggacgaagtagcgggcgccttcaccgagccactcggccttggcgatggcgcgttcgagattggccttggtggcgtaggtcttggtgtggttcttgtcggtggagaaggttacttcgcgggccatttgtcgattccttttggttgaagggtttcgcgtttcgatgagggaatattacgcccacctgatccagaagtaaagcacttttgtaaattacttcacgaacattttcttggccttctgataagacgaagaagtcatcaggcgctcgatgacgtccatgtcagaaaccagatcgtccagaaggacgttgcgccaggtagcgaaccggccgagcgagaagatgccggcttcatgggtgagattccagatcatggattcgcgctcgtcgcggccgagcggaacgattttgcctttggtctggatggtcggctcgccgtcctcgatgagatgccttttccttatgccgaaggccgcgcaaacgtaatccacatcccagtcgctgtcccattcgatggtttcgattttaccatctaaagtctccactatccccttagtgatggattcgacgatcagagtatcaccggtgatggacgctcgaaacgttcccactttaggaccagggaaatacacggtctggaagacatcacaggggatggaaagcttgtatcgactcacgatgatggaggttccttcaccgaatgacgggtcgattcccaggtccagccctgccgcagccagattggcgcggaacggcgcggtgctgatgatattcacgtggtcatcttgccggcgaagatactggaagaaagaggcgtcgaaaggacggctccaagtgatacgattcgcaagcttagccaccagctgctcatagtagtcggccggtgcgatccaacgcttttcagtcgccagattccagatggaccggtccgacaggccgcccgttactttcctggagtacatgttgcagtggtcgatgcgcggctgggaaatgaactcgccgtcgatgtagatagctttgtgtacagtgacttcgcggaacgggatgccggtgagttggccaatcactggcgagcggaatcgcagaagtgcgttgtggcgctccttattctccggcgtcgccgcgtcgatgatttgggcttgaggaaagcgatgcgcggcgatcagtccggcgagtccggcacctacgatgattactttctgatcaggaatcatgatgtgtcccttatgagtgtacaaaacttgagaggataaaaaagggacccattttcatgagtcccttgaagagctagacgattcaggctcagaggagcggcggcttactcctcctcaccttcggaaccatcggcgccctgatcttcaccgtcgtgctcctggccttcgtcatcggccttctcgtcatcgccctggccagcttcgtcttccttcgaagcgatggcaaccagatcgacccagcccatgatttccagcttgctcaggtagctgcgaaccgaagtgccatagagcagatgggccaacttctcgccgaaggattcgatttcgaccggctcaccaacggtgcagtgctcgttgatgtatgcgaacaccttgccgcgagtcgagaaggcctgcggagtgccatggccgtcgccggtcgggatgaagtgggtggcgcgcggacggcgggagccattggacttcaggtcttcgcggcgggcttcggccttcttgcggcgctcttcctgctcttccttgcggcgctgcttctcggcttcgcgctcagccttcttctgctcggccaggcgcttgcgctcttcttcgcgagcgactttctgagcttcctgggcggccttcttctcttcggcctttttggctcgctcggcttccttctcggccttcttctgctcgcgctcggcttccttggccttcgccttctcggcctgctcggcttccttcgccttggccttttcagcacgctcggcttctttggcggcggccttttccttcgccttctcggcgcgctcggcttccttcttctcgcgctcggccttgcgcttctcttcgcgctcggctttcttctgctcggcttccttggccttggcttcggccttctcggcgcgctcgcgctctttacgttggcgctcagcggccttctcggccttgcgcagggcagcggcttgttccttggtcagctcttcgccttgggtctgttcgttctggtccttctgttccatgttcttactccgggaatgtttaaagggatggcttattggcctgtgagaggattatctctaaactaattgaagaagggaatacccttagcctgaactttcctaaatattttctttcgggaaagtccaaactctagggaacttatttatgttcgagaagttcctagcttttacgcaagaacagtaagtattcgattgcgcgagttatcccagtatacatcaactgactataagggatggacggcaagttttcttccaacatggcgacccgtttccattccgatccctgcgacttatggaacgtcatcgcccagccgaagtcgaatccgccaatggccttctgcgcctccagccgcacatcttcctcgaccgaaaagctcaagggattgaacttcacccagcgctcatagttcgtgccgataatgcgaactttggcaaacagcatttcatccggctcgtcatcatcttcttggccttcggggaccggcttgaagtccagcagaattgcttgctcgccgttcatgatgccatattcgtgctggttcccggtgcacaccagcttctcgccgatttccggttgtacacccttgtagccgaggatgcggcgcgccctggcgttcaaacggcggcgagtattgttgtaggcgcaaaggatcacgccatcatcgtccaagaacgtccgcatttcgtcatccgacatgtcgaatccggcccgtaccagaatgtcgtcatactcgcggcagggtaggcgcttgccctggcggacgaacatcgacgctcgaacgatattgccggcgttgcgctcgatttcggtcatgatggtgtcacagctgttctcatggaaaatctggacgccgcgcacaggaggaacttggccaaagtcaccaatctccagaaccggaattcggtgcgacagcaagcgctcttcatcccactcgccgatcatggacgactcgtcgagcacgaccaacttcggcttctcgtcgagcgagtctttgttggcaaacatgatttcgccgtcttcatcttcgccaatcggccgatagatgaagctgtggagggtccgggcattggtgcaacccttctcgcgaagccgcgctgctgctttcccggttggcgcgacgaagaccgtccagtccatcgagcagcaaagttcggcgatgatcttcgcgatggaagtcttaccagttccggcgaaaccagcgagtcgatagacctggcggcggtgcgctcgatcacaccaaccgcgataccagtccacaacggaattgatcgcgtcgatctgctgactgtttggtcgaaagccgaatcgctcttcgatctgatcgacggtgaagttagatgctgacatatttgcgttctccaacgctaggtttaattgaatcgaggctcagtttaagcacgccgtccacagaccagccagtatcacgacgatacttgcggccttgcggatcgacatagaagttcttcgtgcgccgcagcagaacataatgccaagcagctccaagtgcgtggacccttcctttatacgggaaagccttcgcctgctctgcggcctctttggccccagggagccagcggacggtcgaaaggaccaggaccgcacccttaatagcctgggaagcggcgcggccgtcctgtgggctatagcgatgtctatcgaagtctacccagtggtggttgccgcgccggagcttgaccgttcgggatcgcccctcgaacaccaccgtgccttcatgagtaaaaatatgttccgccatcgaatgttcctttataacatacagttatgctttacctctgcgcaggaagagtatactatcagctgactcgccaaagcgagcgaatttaatccaactttacttcggcaggaaagtggccgatactagcgccgccgcctgtactgccctccaaaacagaggatacattaaatgcaagaatgcaagatttcccgcgaccaactcccggtcggtaatccgaatcccaatgtcgacaagacccgcgacccgaacctgaagcccggcttccttcgccgaagccgcgagctggacccggcgctggccgttcgcattcgtcgcgagctgatccacgccgaagcatccgacttggccaaggccggatgggtcaattcacagtccagcctttatggatcgaaagccttcccgcgccattccgtcgttcgcgtgaccggagttccggaagatggagctttcatcggcatgctgatcggcttcatcgagcatcgcgagcatggcgaatgggcggtcatggaagccggaacgaaagaaggcggcgccgtcatcatcccggtcgatcacatcatgcgagcgtcattcgccgaagccgaagagttcgccgaaaagtgggagcggaacttgggatggcgcctcctgcgccagctccgcgagtgcggcgccctggccgggactgaagacgagttcctgcggcggatcatcaatcgatacgttcgtgatcgcacgatcctcgaccaccacaaagtcggcgctgacaaaatctacactgatgcagtgctcaaaagcatcggcgaaacatggccgaagattccttcggggaaattcgtcggacaccgagtcgcgcaactcctgatcgaccacaagctaggtcgagcgggaaccattctgaatgacctggtggacttcctggagaagttcgcggccgggcgcgataaagttctcaacatcgccatctgtaattgaggtgaagggatgaacaataaaccgacgctgaatcaccaccatcaaactgcattgttgtatctgtataacaatccaaatcagccggccttcacaggtcccaacaatcaggcgctcaatgaacttcggcagatggggtatgtaaaggcgaaagaattcgaaaactgggcaggcaccggccatcttaggatggaatggacgctcaccaaagccgggattgagcgcgtcgaagtcgggcttctggggaagtgcgacgcttgcaagggaatcggtcagactttgcttcgggggaagtgtactgtttgcaatggccgcggccaggggtggatcagagaatggtcgcagaagccaatcgaagataatcctcaaattgtccctaagttcgaaaagaccgatgcgaatagacttgcagacgccattgaagaaatcggccggctggaaaaggcactggccgaatccgaaaagcgcgggagcgagctggccgcgagctattgtgacggcgtagtcggcgatgaatacggccatccttattgtcgttataaagtggaacgcgacgccgccttggccgaagtagagcgcctgcgagaatcaaaaggcgatccctctggcagcttcgacagttgtatgaagatgatgtacgagcgcgacgagaatgcaaaacgactcgacgccgccttggccagggtcgcggagctggaagggaagttgacggactgggtgcacgaagggttccggctcaacgaggcgctagcggtcgccaaggctcaacccagcgtgccggaggggtggaagctcgtaccgatggacccgacatcgcagatgacctctgtcggtcagtccctgcgttatgacgcggtaaacagcatcggcgagatttaccggcaaatgctcgccgtagctccctcacccatcgatccggccgcgcatcctcagccgtgccagcaaccacaggcccatcctgcccgctgcgggtgcgagcggtaagtgccaagcaaggagttcatgtaatggaacagcagaaaccttcgaaagtagatggagccatcgccatgaccgatctcgatattcgcattcttcgaaaggcgaagcccgaagcgcaggacgagtgcgccgtgtccatgttcgcaacggcgatccgccagaaactgcagcgctcccgcgataaaggccgaggcggctggatcgactgcgacgaagatgttctgatcaacggattcgccgaacatgcgctgaagggtaatgagaacaacctcttggacctggcgacgttcctgatgttcatgtgggtgcgcggcatcgacgatgcgaagattcccccggcgctcgaaaaggcgcggcagcacaagatcatggaagcatggagtcgggtccacgaagatggactaaactccgccagaaaggcgagtgctgcgcgacagttcgtggaagtgcctcgacgcaaggggcgcccggagcgacttgcatgaagcctcacgaaataagactggcacaggccgaagagttcctgcgcgaactcggccgagggattccggacgacgagagggtgatggtcggctacgctgaagaggccacagtccagaccgacgagaacggccgcaagctcaacgccggctggtggcctgtgccctggaaggaagggaaatacatcaactccagatccaacgcctacgcgtgcatctcgtcgtccatcaagacgcccaacccgaagaccggccagatgcgatactggcgcggcgaagcctctttcggccacggcctggcgttgatggtcgatgacatcggctcaggcaaagggtccaagggtgacttcgaccgcgacgagttccgcgagcgactggagccgaccgcgattgtggagacttcgccgaacaactaccagttctggtatttcttcaaagagccgatgtcccacatgctccagttcaaggcgctgctctattcgttcgtggaccaggtgctaaagaaaggcggcgacaacaccgtcaaggacgtcagccgttacggccggatgcccttcggcttcaacaacaagcgcgggaaagacggcaagttcaaatatgccgacgaaaacggcaagcccgaactcgtgcgactgttcagcgccgactattccaagcgctactcgccagaagagatcgcccaggcattcggcgtccgcatcatcatgccacagatgaagaaggtggagataaaccgcgacgattgggtgtatgaccaagtatggctgaagtatgccgagcacatctgcacgaaatacaagatgggcgaagcggcaggcggccaggtccagcagaatatgtccggcaaataccgcatccgctgtccctggggcgacgagcacaccaacggcgatcctttcggcgcatactttcgcggaccgatacctggcgccgagcacgaatatgtgttcggatgcgggcacgatacttgccgcaaagagcatcgacggacatgggcggccttcactgatgaagtcgtcctgccctatatcgtagaacaattggaacgcattaaccgccgtcacatcggcgaggagtagtaactatgcaaaatgatcctggaattctgattaccgccatcggctcattgctgctcggccttctcgtcttcttcgaaggcctgaatggctggaaaataccagtgtcaaactttctcgcgtcgctcttgtgcttcttcgtcggcctttctgctttgacgttctggttcgtcgttgcgtttgacgtattttaatcgacgaacggtacagaaattttcggatggggacggaacttattagctatgccggtttaggtaggagataatagccgtccctttcgcctcaatgtgtagaggcaatgttgaatccgatcatgtaaagcagaaggcggcaaacctaacatgattatcgacgaagataatatttttgatgatggcgaatcagggtccagtgagtttgatctcacgcagatagaagatgctggaatggaccctttgatgaccgccgcgagcaaggcggccgatgatgcgattgcgaggaacgaaacgcatcgcgcgcaaaaggcggcaagatacgccgaggcgtatgctgaaccagacttgaggaagcgagcgcggttgttgatgctcgaccaggcgttcgatcttccggtcagccgggtggtgaaagggccgttcgatgacttcatcacgaaatacagctcgacgtcagacagcaactacctcgcggtctatgatacgttgttctgtaagggcgacggaaccgtcccacacccgcacttcgacgagtttcgcggccggctggtggaccatcgcggcgtggcgttcaacaacaagaccctcgatccgattgacctgatgggcgccctcgcggctgcggccttggacgatccctcgattaagaagacgattgagacttgctgcgtttgggcgcgtcgatatcgccgcaactcgctgatcgagacgtttgagaagaagataccggattgggacggcgaagagcgaattagcacgttgctgatcgatcttttcaagccgttcgacaccgaattgaaccggatggtaagcaagtatttctggctgagcttgtactgccgcatcaactaccctggaatctcggcgccgatctcgttggcgttgattggtgggcaggatgcggggaaatcatatttcggcttgctgatctgtaaggagctgtccggtgggcgcgatctggctccagttcagctcgacctgagccgacacgaccagacgccgttcctgcgcaacatcaccggcaactcggttattgcgaacgtcggcgaaatgtccggcttcaaaaagggcgacatggaacgcatcaaagaattcctggtgcgatcttcggatacattcgaccagaagtttgagccgggcgaaacgatcaagcggcaatggatcaccatcatggacggcaacggatacgatggactccagcgggacgactccggcaaccgtcgattctatccgatgttcgtggcacaacttcccgacgaagatggaaagccgaactgggttaagccgggcgatggtaatgaaccgttcaaggtggacttcaccgacttcggccgcaaattctggcaagcgatggctgaatgccgcgcatggatcgaagagcatggtgtcgatggatacctggatatggtgtcggaagcgaaccgcgaagtgcagaagttctctatttcggaaatggagaatgcgcgtggcgtggttcgtgatgacacgattgatatgtatctgatcaacgtgctgatcagttgcgagttcgaagagatcaagcctggcaggaattcaaagaatcctggatggagagtcaacaccatcgaaatcctgaaatggttcgatattctcgccaggaagaagccgatttctcgccacttaactccgcacctgaaggcgctaggatttgttccgaataagaatggcttgaatggatggtgcctgcctgtagataaggttgcacctggatgggcgaatgacatgcacacgacgctgccgccgttcaatgatgcgcttgtgtacttgctgaggaaggtggattctgaggtgactgatgaagaggcaatgattcaaatccgcaaagttaaggccgagcgcgcaaggatattgggcgaggatttctgatgtagtgatggagtgtacttggactaggccgccttcgggcggtcttttctttggtacggagtacattcgatttcggttgtggccgagtgagattcggaagctatctggtaattagggtttggtggcgatggcgcgttgtggaaagttcgtggacggtggatcgaggattttgtgtggtgattggatgaattgtggaaaatcgacgattctgactggattttttgggtcggccgcgtcgagcttaccggtaatttacgaaaccggtaattagggcctagccttagtgccgcgtgggtttgaagggcttaaccggatactttccggtttcggatgcagccgcaaaagtatactacagcgaaaat

SEQ ID NO: 6 - DNA, 64562 bp, synthetic construct
  source 1..64562: mol_type = other DNA
cgattgcacaatcctaatagaaaaaatctatcacggacgttacctatctttaaaattaattaaattaatagtaatttggtaatttggtatactttagtatttgaaagcctcgcggcactaagcctgtacacttcccgtcaagtttccgattcctctcaactcgcggcagggtcgccggaaacttccggacttccaatccatgggtcgcggcaacaccacggcggactaagcggcaagtgccaaaactcgacgaacggaaccggaaatttgggagcgcgatagaatcgctcagctggacatatttctaacattcgatttaacactcaacccaaacactcaccaccaccatcgcccgccaccggccaatccgatcctcacccaccagcagaccgcccatataacatcctataacaccacctaacactcattcaccatcaaacccacccagacctacagcccacccacaagcagcccatagacgcgatccctggccccatagtacaatcgcgccatactcagtgtcgcgacagagcaccaggccccatccaccaaccgagccaccgcgacgactccagaatcgaactcagggacgcagcaccaaatgaccaaattctacagccccgacgatctagtcacgccacaggaattcgcagacccgcatttcgcggcgatcaaccaaaagcgattcgacctgtacatcgacctgcgcgtccaaggttacagctcctggcgggtcttccgggccatctggggcgaggagcatatggacggtcccgcgcaggcccgcatcttcgcgatggagtccaacccgtactatcgcaagcagttcaaggccaagttgaacgcgaccaaaacgtccgatttgtggaatccaaagacggcgctccacgaactcctccaaatggttcgtgaccccaccgtcaaggactccagccgtctgtcggccatcaaggaattgaacgttctggctgaaatcacgttcgtggacgagtcgggcaagaccaggatcggtcgcggcctggccgacttctacgcatcagaagccgaggctcagaccgccaccgtcgctgctgcggccgaagccaatggctatgtgccggaaggcgaagagggagatttcccgtctccgacgccggaaccgaccgaggaagaccgcgccaaccccatttagacataaaataacatcgttctaggcccgaatcggaccgaactaaggcgacggtagcgggaagggacgaaaaacgattctagggcggttctaggaagtcgtagcctaaccctcagaaacgacaaagccccggactctagttcagaatccggggctttctttgggcgccttattcgccagcttcgatgatttcgaagttgtatttgacgccttcgtgctcgaaagtcattttgccagccgccttcagctgcatgcggaatcggatgtgcttcgaagaaggcaggccgaactcgatgaatgctgcgttggtggaccggaactcgccgcgcttgcctttgacagtcacagccacgccatggcgctgagtgcgtttcttggaaacttccgggtctttccaggagttggcgatggctgccgacaggtctttggtctctttggccttctccggcgcgttcttggcctcttcgcgcatcttccggatttcttccagtgcttcctcttcggtgatctcttcttccggcttcaggttctcttcctcggccttctcttcttccttcttcttggaagtgcgggttttgtaaaccttggccggggcttcctcttcctggaaggcttcttcctcggccggcagagcattcagaatggcgaggcagcgacgctcagcggtcttgcggtcggagaagcgcttgacagtcgcatcggcgttgtgagcgttgtagaaggcgaccagctctttcatttctgcgttctggatgtcgccgaaggttttgatggagttggtcattttgcgatcctctgttttggaagatttctttcgggcttcggtttgtcgccccgttgaaagagattatgcctaggtcgatgctgcgtgtctacatttatttcatcagattgatcacaaagccgacgaacggttgtcggatgtgaaaacaccgcagaacgtgctgcggtgtttggtgggagagggtgatggtcagaaagtcgggactgtgatcggctcgattggcgggtcgccgggcttgtcgttgtccgatgcgggagctggactggacgggatcgcgccggaccgaagcgacagagattccgactgcctgctcacctgcggagctggaggcgcttttggcgcatgaggcgcgggcggaaccggcaggcctggaggagccatcggcgccctgggcggctccggaagatcgggcagatcaggaagactcggcgctatgccgccaaaggaccgctgcgactgctgcgccggattgtcgccgaacatttcggcggatcggttgtctttgatgacttgacctatggccgtcttgtcgccgcccggctctgccggatcgaagcccgtgaatggagtggtaacgcagcgcatgttcggatgggtaggcagatttttcaggactatggaatctgcccagacatacggattcttgtggttgccgggtccgtggctgatgatctcggcaggaatgtggccgttcatggtaagccattccgccatcccttgccgagtcagcgggcttaccacgatctgaccagtggactcttcggcgacggcgaacagatcaccgagcttgaatccgatgatcatatagttggacctggactcgccgacgaaagccagagtttcgatgttgtggtggatatcttggtcttcccagatgtatagcatgatgtgctccttaataaggctgcttgttgtgctcgaccagagacataccggccggaactttgaagcggatgtactcgatcatcatcgccgtgcgacgccgattcgtgccagaatcgtcattgacgaagagttcgcactgatgcggaatgactcgcgtcaccaggtcttcgcctttcgagccgtagaacttgatgtaggctgcgacgcctcgcgtcatgttcagatgaatcgtttcgcagagtcgctgcgcggccgacgcgctagacagatcggtcggactgaccgtcacccagtaatggccattttgttctttatcggacatcaactgccctccattgataaagcccctgccgagtagcagaggctggttgttagtcgcgcttcatcaagacgactttatcatatgcgcgatacttcccgcgccagtcttcccagtagtcgccggccgggcaggtcagctccagattgatttcatcgcgccagcattcgacggaaaggacaatcgccttgttcctggctgcggaatctgctctgagcttaatcagaatttcgtcgcccgttttcagttcatcgactcttacaactttggccatgttgttctcctattggttagtgccacgagggggggggcatgtagcttcgatgtcctgcaagcagatggtgggatagtctctggccagcttgagcttgaagtccatccagtcagcaccgatgagtgccgcgacttcgatgatgacttcgtcgttcggcgtcgcgctagattgcaggactatagcccgacgccgaacgggatcattgacgtcaacatgggtgagtatttcgtcgccgacgagcagctcacaggccatcagtattttggccataacacactcctatgagaaggcgcggccagggcaatcccgaccgcgccgatggattaacgtttgtgaaggatggaaacagagtcaacgtcgaagatgctgatggtacggcgacggcgcttgttgctgcgttcatggatgcgaatcaggttcgcaactggagcttcgtccaccacccatacatcttccggcttctgcgtccggagcaggaccgtcacttctgtattctgggcgaggccgttgcaaatgaacgtgaatttggacgagggagttctgtacatttcgagattcctttttggacttcgggtccgacttctcagtcggtgaagggattatgcctgatcatcgccatcgagtaaagcacttgtgtaccaattctccagttaaatggaaccaaagcgcggtatcgctgattgctacgctcccacgccatggcccttcttgcttacagacttcgaaccacaagctgatttccatccttgccaggactcggccgaaggattcgagccggctccggctagccaggatgtcgacattcacccgccgattgatttcgtatcggcgaccgttgatgacgaagaccaggcgcagggtgcgtccgtccagacattcccagtatttggtttcataccgaagccagtagcgcttgccagccgggcctaccatggtcatttaatcgatgctcctggccgcgccacgcggtccggtcggcggagcaggatcgaaccgccccaggatgtagtcgggccggcgcgcttcctgaggacacacggcgagaaggcgccatcctgcgtccagggcatactggagttcgtccgtgcagcagtcttccttgagcagcagacgattgatgctctggagattcggaccagggatagcagagccggtgaccgagttccagccttcgatgccgtgcaccatctgcggttcagatatgtagcctggcgagcgtccctgtagacggctcgcggccagctccaggcgttccaacatgggtcgcagagcagcttccgggtcaacgtcgtcccagagaaggaccagattgatgatggtgtacggatagtccttgtccagatcccaggctgaagcggtcagacggcccaggccgatttcattacacatcacgggaacgtcgttgctccatgtggacggctcccagttccgctcaccaggattcccgattgttacgccttccaggttccccaggagaacttggagtttgctgacgtattccgcctccagcgttttccgctcttcgtcggtctggttgtggcgatagaaggatggagggctgacttctgcatggtagagtttcatggcggttcctcggtttttgaaggcttgaacgttagaaaatggtgtcgcagtatttctcgaaaggactctggcgcttcttctcgcagatcgcgcaggtgatttccaggtcgggcagctcgctgtaagtcttgccgagatacagccagcgcttgcacagactgcggccgtccgacgtgaagaagtggactttgcgagcattgccgggctgcgcccagccaccttgatcgtttttgcgcttgctcatggcgatatttcctttggatttggaatccatgccgtccgttcgctcacccaaactccgtctacatagatcaggactgacagagcagtcgcgccaaacccgaagcctacatagaatgaaccgggatcgatttccatggtggcgctattggtggcgctattgaaatcgacttcaaagtcgaacatcgaatggccactcataccatcaccatgtcgatttcattgatgaagaaatgaacatcgacaccatcggcgcgaccggtgtaacgcagccgagtcgtcccatcaatgtgggcttcctcgacgccgatgacttccaggatggtgtcgttgcagtaaagcttgacgaacatcttgatgccggagccaatcgccgcgaaggcgatctgcttgtacaggtcttgcttgatcatgctttacgctcctgtttgcttatgtattctgccctggcttcggcttcgagcttcgcccaaagcgcgttgtcgactggaccccagggaccggtctgagttccgtccaccggtgcgaacttgccaccggcgcgacgatacttggacagagttctcaggccgatgattttctcttcttgggaatcgagttggacgatcacttgacatacttcccgaagtagcaggtgtggaggatttccaggcccttggagatggccttcaacttgatgccgaagttcttgatcgcccagtccagatcgtggtgatagaactcgattatgttgacgatttgaccggcgctgtagtggatgacgcgaaccttgccattccagcggaaatcttcgcggctgtttacggcctcgacttcgatggtgtcgtttctgccgatggtccacttggagctagtcatgtcgcaatcctctttttggagtgtttcgcgtttcgatgaggtaactatacctcagtcacctcatcgagtaaagcactttttgaacaattatttgaattttctggaagacagattcatcggccgattcattcaatcaatggaaccgagactccatccgttgcgggcgaaggccgagcgaccagacagcttattcgtaggtcatcatcttgccctcaaggggtcatggcccggcgaaccgggcgggtgattacttgcgatatttcttaataaggtcgtgtgccatggcgatggcatcagaataggtttctccgccggcctccatttccttcacagccttattgcgcagtgcgttcagcttgttgccgttgatggtgcctttttcatccttggcagcttcgcaggagtcgcgcagagttttcagggcctggtaggtggcgttcatcgtcttgttctccgggtttcgcgtttcgatgaggagattatgccgatagtcagaatggaagtaaagtgtctgaatgaagaaatttctgaacaccgacgaacggttgtgctcgaccgtctgctgcggtgtcgatatactcggcctattgcgaacaatggactgattaaatgttcaagctcaatcctgcactgcgagcggtctggcgaactcgtgcccgttacaaagtcatctatggcggccgggcgtcttcgaagtcgcacgacgctggcggtatagccgttttcctcgcggccaactacaagctcaagttcctctgtgctcgccagttccagaaccgcatcagcgaatcggtctacacgttgatcaaggacaagattgaaaactcagagtacaatggagaattcatcttcaccaagaattcgatcaagcacaagagcaccgggtcagagttcctattctatgggatcgcccgtaacctgtcggaaatcaagtccaccgaaggcatcgacattctctggcttgaggaagctcactacctgacccaggagcaatgggaagttatcgagccgaccatccggaaagaaaactctgaaatctggatcatcttcaaccctaacgaagtcaccgatttcgtgtatcagaacttcgtggtcaaaccaccgaaagactcctgcgtcaagatgatcaactggaacgaaaatccattcctcagtgagacgatgctcaaagtgattcacgaagcctatgagcgcgaccgggagcaggccgagcacatttatggcgggattccgaagacgggaggcgacaaatccgtcatcaacctcaagttcattctcgcggccatcgacgctcacaagaagctcggctgggagccggccggatcgaagcgcatcggcttcgacgttgcggacgacggcgaggatgcgaacgcaactacgctcatgcacggcaacgtcatcatggaagtggacgaatgggacggcctggaagatgaactgctcaagtcgtccagccgcgtttacaacctggcgaagatgaaaggcgcctcggtcacttatgactccatcggcgtcggtgctcacgtcggttcgaagttcgccgaattgaacgatgccagccccgacttcaaacttatctatgatccattcaacgcgggcggcgctgtcgataagcctgatgacgtctacatgaagctgccgcacacgacgatcaagaacaaagaccacttcagcaacattaaggcgcagaagtgggaagaagtcgcgaccagattccggaagacttatgaagcggttgagcatggaaaggtttatccattcgacgaattgatttcgatcaactctgaaacgattcacccggacaaactgaatcaactgtgcatcgaactttcgtcgccgcgcaaagacctggacatgaacggccggttcaaagtcgagtccaagaaggatatgcgcgagaaacgcaagatcaagtcgccgaacatcgctgattcggttattatgtcggccattctgccgatccggaagcccaaaggcttcttcgacttctaaacacagaaaagcccggatcgctccgggcttcgggtcttactcggtgtggttcctggccctgagtgtcgacgcaacggcctcgccgactcccagggcttcctgaccggccgcgagcgcttcggcttccgactcgacgatgaagtcatcaccttggccgtcgcctggcggaacctcgatcagcacggcttcttcgccttcgaaccgcaggtcataagtcttttcgacggacagtccgtaacgggcattgagcgcatcccagagctgggcctcataggtccgcaggtcttgcagagctttttggtggctgagcatggccatgtcaacggcccgttgcagggtttcgtccaggacgttgaaccgaatgcgaagcgcacgaatccgctcgacggtttccgcatccactacatgtctttcgatcattgcttttcacctttgctgaatgtaacgttgtagccgttgtcggccaggtaggtcagggcgccttcgaaggaagttccgacgaggtgcttgagcttcatttcgcgttgcgcggccaaccagaatgcagttccggaaaactctgcgcggccttcggacaaaaccctgccatcaggcccggcgatccgtacatggacggaagatagcttgatcggcatcagtgaatacctccactggcttgcgacggcatgctttcggcgcgagcggcttcgcagtccgggcagggacaggcttggcggacgcgctccaactcgtcggcatccatgacatagagcttcccgtcggcagtgtcatgtgccatagcgatgttcgggaagtcggttgcgctcaggccggcgacagtgcggatttcgtcccagagcaccagatgctcggcattcagtcgggcggcgagcgcttcgtgctctttagcgacgcgcgccatgaactcgtccatccggaatgcgaattccgcatcgatggcacgggccgaggccatggagctgagtcgaatcggttctttcttcatgatgattctcttttggttggtggttgttagctgctcaggcctattcaaggccggctcaacgcggcgaagaacaacacgatgaaaaggagcgaaaagaatgcgaacgccagagatattccggaaatcagctttgctatgccctgcatctcagttgccttggcagcgagtctggccttagcctcgataacttccggtcgtggcgactgaacatattctaaggcctcttcccgcgctttttcggcctcgaccaacctagggtcgcgggccgagacttcgctgtgccctggcctcgcgggatgggcttgcagcgatggagggagttcagccgccacgactccatagtctgcgcaggcccaagcgatcccgatgaggatcgcgatgatggattggacgattcgcagcatcactttgtcgctagggaagttcatgattaatcctccaccgaccgaacgatttccatgttgcgaccggcattggtcccggcatttaaggcgcgccgaccgtcgctatcgtccagacgcatgagcttagtaacattcgatttcttgtagccaggatcgccgaaatgttcgtgaaccgcagcttccttcaccacaaccagagacgttccggcagaagataccagctccatgcgcttcctggtgatggattgaaggcgatagctgatttcctgggtcgcggccagcttgaattgcgcggcaacctttacgttgaaacgttcgtacccttgagccttctgatactcccggcacagacgatcaatggcctcgaccagggagttgaacatgttcaccgccagctcaacgtccgacttgtagcctttaaagcgtacggcatgaccccagcgcttggtagtgcttccgtcgcgggcgcttctggatgccttggccgatgctcgatggttgttgatgccaccggcgaaatccatgatgcagtcattgtacgtcgccacggccaccgagaagaacttcatccagttcgggattgctgaatagtaacgagtagcaatttgctcatcgaactcttcgcgaatctcgccggtcgcttcgaagtcgtgaaggtcatatttgtccttcaacttcttcacacgctcggctgcgatggccgcttcgtgcgggctggaggagtcggctgccatggcggtcagtttgcggatgcggtctttcgccttctcgatggcttccggagtgaattcgttctggtcggtcatgatcggttccttttgtctgaagggtttcgcgtttcaatggagctattctgccttcattcagaatggaagtaaagcactttcttccactatttcggcatcgactggaaataattccaagcccagtcgcctgcggccaggacgaagatgatgaccgcgaagaacgcgattgcagcgaccagttgcgcgcccggcttcagcttgggatggctgagcttgggctctacgggaacgcccggagtgctggcgctggaaccggcgtctcctgggccgtagccgattccacgttcacgcgcctggagcgacaccagggatttcagatactcggtctgcttttgcgactcttcgtagatgccggcgacggcgaaccagagcgcgaacactacggccgtgcatatgacccaggcgccggtcagcaggagggccagcggaccgatgaggaagacggaagcggccaggacgatggcgccgccccagatgatgaagccggccagaccattggtgatgtcgatacagaactttttcattttcagattccttcggttacgggatggaggggatttggaactctgcgccgccgagaacatcaatgacgacttcccagagcgtcggaacagaccactggtagcggtcgaagtcggtttcaggatcgacgcccagcgtcacataggaagtggcaggccatttatgaacgctgcctttccgcatgagagtcgccacgcgaccgtcgccgagcggattcgtcttgtgtggaacataagcagacatcgaatattgctgcccggcgtcgacaacaaatctttgcctctggcgcagacatacagagcccacaggattccagccgcgaccgccgcaagcctggcgagccgaagaatgctcataaccttcgtgagtcatttcgccgaggcaagccgagcgatcctcgtaatagttgatgttctccatcgatccaaccagcaccagagactcgaaatcgaatcgatgatcgtggatggccgagtgattgaagcaaagccgacgcggcagctccgggtgccaaacatggaggcgaccggccggaagttggacctgaatgaaccccaggccgtgcagagtgatcttgtccttcatcgggtcatggacggtgctcatggataatcctcagtagcagaaatgtattgtgagagttacgattgacaagccggtcgcccatagaagggcgaaccaggccatggctttgatggtcgtgtagatcatccgaagaattttccggcacagatcgggccaatgcccatttcgatggatgcgtggttggtcaactcgcgaccgcagcaggagcactgaccagtcttccgaccgtaggcgactgccgattccatcggcttttcgaacatcttcagaacgtcgtcgtgctcagtatcggtgcagtcgcgactcttgatgaatttgccattggtgatccggccgaggtagatgtcgcccaagacgtacaagctaccggcgttccggctgtgagcgctagcctctttcacaacaacgatgagaggctcctcgccttcgccagccaggcggattttcggacgcttgattccagactctttcgccttctcaaatgctttctcgataccggaaatgtccagagtcggcgcagcagcttcctgcgcggccactttctcgcgatacttggcgaagttttcgatggcgcgcttcgcagcagcgatctgattttctgtcaaggagccgtatttatacaacgactcctgaaggctctgagcgaagctgaaggaatttccagtccaccactcgacgatgtccgggtgcgcggcttcgaaggccttaattttgaggtcgcgctcttgcgcagcgctacagattttctcgatgcgcttttctgccgccttagcacggctcttggcgcgctgctccgggctggttttgtactctttgtatccgacgccgccgcaggcaaagcaggcgcgaccataagacgaagggccacggtacaggccggtgcctgcgcatttggtgcacttgtcgcgatacagcttcacttccttccgggagttcgggcgggcacccattgacacgtcttccagggtcttcggcgcttcgttgttgatctctacggtagcgaagtcatcacccaggtcttcgaagccggtgaacagattctctgctgcgttcatatcgattctcctgtttggaaagttcgtttcgatgggttgactatacgccagaaatggcaaaacggtagcactttcgcactaccgttcgtcgggtcgcagacgatcaataaatatcgctattgatcttgaatccatgctcagcgccgtcgttgtagtcatactcagcatagccgtcgcagtagtcgttcaaatgctgaatgatgccaagaacgtcgttcgcggctttgtgtcgtttcgcggcgatggtcttggcgatgctgtcgccgacttccagggtttcggccgtccggcgatggatcagcaggcgactccaaagatagagccggacgcgacgaataatgtcgcgatggcgctccagtcgtccgtgcaactcttcaatttcgtgctcgcgagatttgaatactcgtcgaagctgttgaacttccaactccaaatcggccttagtagccatgttcacctcagaaaggaaaatcgtctgagactccaggaagctcgacgattattgttgagcctgatcgatccagaatgcatccgaccgagccggccctgtggggatagggtctacacgaactataacaggtgacttccctgactattcgccaggtcgatcctccacaccatctgcaaattggcgggcgcttcgacttcagttttcgttcgtccaggacggcgcgtctgtcgcaggaaaggcagcgtgccttaaccgacgtaaccatgggtatagtcgatcattcggatgagctcatcatcctctgattctctcgacttcagaccgcccaaagagtccgactggccgtcgcatttcgcatcgatatccatcgaagacaggaatcctgccgatactgcgatgatatcaactttgtcgccggccttcttgaagccttcgcactcagcatcgagggccacaattgcactcgcggccatgttcacatgactgatgaggtcggggaagatcacaggaacttcacgcgacatgccacggaccgtcagcttcaggactacatacttcatactcactatcccttttgtatgtgaggaaagaacttgctgttttccggatggtgaaaacgctcggtcgcaggcggtctttcttccggacattgaatcgtcgaaggcgggaaaaccgcgccggcgataatcgccgcgaggagtgcagcggatgtcgacatccagagggcggtttccaggctcacccgaacttccggacggcgcggcttcattcgctccaccctcttcctggctcataggatggcagaccgccgccgcgaatccaattgtcccacatacggttcagaccagtcggtggctggggcggcggactgtagaatccgggagtctcagtgctgctcaggaaataaggcgtgcaaacggcctggacgaccaattggtgccgctcccacatcttatcaatcgctcttagcatgacgtcttcgcactgagccttactgtcgaaccgtctgctagtatggtccggcatctggacgcagccgtctccagtgcaaaggaaagaagtagcaatccatacggtgatactcgccatttcgtcaccctctttagttgatgagcagagtctattccatctgctcgccaggagtaaagcgcttttcgtcgggataaatgccgatgatgtctgcgtcgagcatccaaatctccacggacggatcgtcgctgctgatttggtacaggttgtacagctcccgtccgccgacagcgcgctcgcctcgcggctcaaccgccagaaccttgccgtgcccttctccgtgctcgtccaggtatatgacgtgatcgccgacttcatagctttctttcgtgacgaggcgtgaacgcgagctgttctgcgattccactacccaggaatccagcacgccgcctttcatgtcgcgaaacaatcttgccgatttgtcgcaatcgaatacgcccagaacttcgccgtctttcaacacgatatgtacgattggcaaaagagtattcatgtttaatctccattggttgataattagagtctaatctgccgaaaagttcccgtaaagaattattttctcataactgattagttgcaactgttaatctgatgtatatgtttgaatctcttttgaacgtttgatgtttcccctataataagcgcacacagtcaacaaccacgtggaactacaatgtttaaactttcctggatattcgggcgcaaaaaggataatgctgcctgttcggaatcggcgccggagaaagtcgcacaaatccctcaacacgatccgctcgaccccatgatcaagctgggaaggattcgcggctggaacgtcgagccggagaaaaccccggtcattcgtagtgtgaaagatttcctggagccgggcctatctgtagcgatggatagtgcgtatggtgatggaccaactccggccgcgaaggctgctgcgggcggccagaatccctatgtcgtcccgactatgttgcaggactggtacaattcccaagggtttatcggataccaagcttgcgcaatcatttcccagcactggttggtggacaaagcttgttccatgtctggggaagacgcagcacggaacggatgggaactcaaatcggacggcaggaagctatccgatgaacaaagcgcgctgatcgcccggcgcgacatggagtttcgcgtcaaagacaaccttgtcgaactcaacagattcaagaacgttttcggcgttcgtatcgcgctgttcgttgtggagtctgacgatccggactactacgaaaagccgttcaatccggatggaatcacgcccggctcctacaagggaatctcccagatcgatccatactgggcaatgccgcagctcactgctggctcgacggcagatccgtcttccgaacatttctatgagccggatttctggattatcagcggtaaaaaatatcaccgcagccacctagtggtggttcgtggaccgcagccgccagatatcctgaagcctacatacatcttcggcggtatcccgctcacccagagaatctatgagcgcgtgtatgcagcggaacggacggcgaacgaagccccgctgcttgccatgtcgaagcgaaccagcaccatccacgttgacgtggagaaggccatcgctaacgaagaggccttcaacgctcgcctggcgttctggatcgccaaccgtgataaccacggcgtgaaagttctgggaattgacgaaggcatggagcagttcgacacgaacctggccgacttcgacagcatcatcatgaaccaatatcagctggtcgcggccatcgccaagactccagccacgaagctcctcggcacttctccaaaaggattcaatgccactggtgagcacgaaacgatttcttatcacgaagaactggagtccattcaagagcacatattcgacccacttcttgaacgccactatttgcttctggcgaagtcggaagaaatcgatgtgcagctggaaatcgtctggaatcctgtggactccacgtccagccagcaacaagccgaattgaacaataagaaagccgcgaccgatgaaatctacatcaactctggcgttgtgtctccggatgaagttcgcgagcgtctgcgcgacgatccgcgttccggctacaaccgactcaccgacgatcaggcggaaaccgaaccgggcatgtctccggaaaacctggccgaattcgagaaggccggtgcacagtcggccaaggcgaaaggcgaagccgagcgagccgaagcccaggcgggcgccgtagagggcgcaggcggcccggttcccgccgctccacgcgggactaagcctctcgcgaaagcggccgaggaaggggccagcgaggccgctgaaccgccgtcgaggccggaccccaaggccgagctgcggaacttgttggtcgatcttttgtcgaagctccaagacctggacgacattaaggcgccggacggcgtagacatagagcacaatgatgcgcctggcttaaagcgcacatccaaacctggcgtgtctggtatggagccttcggtgttttcgtccaaccgcatcgtcgggcctcgtgatcattcggaactccagagaatcaaggtgaatggaataaccaccttgatcgaaaatccgcgcggaagcattcgacaaggcaaggatgggagttggcgagttcagatgaaacaccactacgggttcatcaagggaacgaaaggcgctgatggggatgaagtcgattgcttcgtcggtccgaatctgggatccaaacgggtcttcgtcgtcaaccaagtgaacaaggaaggtcagtttgacgagcacaagtgtatgctcggcttcaacaacatcaacgatgcgaagtctggatatctgtcctgcttccgtccgggctgggatggtctcggctccatccatgaagttgacctgcccgctttccgtcgctggctggcgaacggcgacaccaccaaaccattcgggggcgagtgatggcattcaaggcctccaaaaagcgcgagcgccgggcacctcttccagtcggaagagggaagcccataattccatcggcaggaatcgaggcctggtatcgaaagcagatgaaggatatgtccaagctcatgatctccgactatcgaaacgagattgagaaggcgctgtcccagcctgcggccgaacgtttcttcgccagagacgaatccgttaacgtcctgttcaagatgacccttcgaagcctacagcagcgatggagccgcatttttgaaggtttcgcggccaagatcgccccggagttcgtcaaccggaccgaagaagccgctaccgccgcgacccttcacagcttgtcggtggccggcgtcgatcagccacgagctgcgtataatgagagcgtcaggaacaccctggaggccgcaaccacttacaatcatactctcatcaccaagatccaagaggaagtccacgagaagatttacacatcagtaatgctgtctctgacttccccgaatccggaagagcaaggaacttccggcataactaacgcacttcgcaaagtcgggaagttttctgaagatcgaatcgaactcatcgcaagagatcaaacaagcaagctttacagttctctgagcgatgagagaatggcggaaaatggagtcgaggagttcgagtggctacactcttctgccggcaagactcctcgccacacccacctggagaaagatgggaagaggttcaagctgaatgaccccagactttgggagggtccgaaagcagaccaggggccgccaggatgggcgattaactgtagatgcagaaagataccagtcatttgacatcgataggagcgctatctgccgttagttcatggcacttccaatgaggctcgttctgaaaacatcaagcgggaaatcgaagccggtaaagacccaaagcaggctgcggccatcgcttattccatccagcgcagtgagaaagggaagacggcgaaagattgttcgcctgagctcgtcgccgatcttcgcgccctggtggacgctctgtcgaggctcgtgaaatgaaccgcaagacgtgccggcgccgactcgtggtcgatgtaatcagggccaatattcacggcggattcttcagcctgaagtttgccgccatcgatttggcaatcatcggcgtcgccatcttgatggcttttggccgataatgctgagaaaaatctggattctgactaaaaattctagtccggatagccgcaagttaccgtttacggaaaatagcagtaatttggaaagcctactgccgcgaggctttaacagagccagttcctaatttccgatttagccgcatgcttcaaaagtatatagcctgggaaattagaagtaacgttacaatagaattcatctataagtaacgttataatataacgtcaatctatatgctctagacgtattgaaattcaatttttaatcgggaaattggtaatttgaattagtttaggagttgaaagcctcgcggcagtaggcttggacaaatcccgtcaagtttccgtggtcaaattactggattttcgcggctgagggtctggtaattagatcataatacaaattataatgtaagataacagtcgcggctacatctaattattgttccgcttatttacccttagatgtactgcgtatataatacagccatagtccacgactcttcgaattaacgatgacaaagtcgaaaagaaaaattgacgaaaatggatatatgaccatcgagggctgcccgatcagctcttatggcgttttccaatattctgctggtcagctcggtcttccgggcgatccgacgcggattgtcaacgtatatcgcccggagtctgccgttagcgatccggagtacatcgaatctctgaagaatctcccgttgatcgacgagcatgagatgctgtcgggattcgacgatgatgacgatagcgtggcccccgaagacaaaggggtggagggcatcatcacatccaacgcttactacgaagccccatgggctcgcggcgatatccgcatctattcccgcaacatgcagaatcagctggaaaggggcaaagaagatttgtccctaggctatagttgccgctacactgagcaacccggcatctggaacggaacgccttatgaagtcgtccaggacaagatgcgcggcaaccacatcgccctggtgaaagagggtcgtgtgccgggggccagagtattggatggtctgtgttttgaccatctcagttttgatttcagaccatccgatgagggtaatgaaatgagtctcaagaaagccaagcggaagccccctgtccagcgcgtagggcaagctgctgactcggcggtcgaagagttgcgcgccctgtggccgaagctatctgcgtctgtccagaagttcctgggcgaagaagagcaggagccggagcatcaggaaggcgcagctccggccgaaccgaccgacagcgagcacctgaccgagcatccgactctggaaggtgctcagaaggatgacgaagagcaggaagaggagccttccgttgtcgatccggccgtggccgccgtcgagccggagcatcaagaaagcgccgcatccgaaatgtccggtgaaggcgaagtcgccgaactgatctctcaggtcaaagccattctggctcgactggagggcacggtagccgaaggggcagacgaagagcatggcgaaggtcaagatgtcgtcgagggcttggaggagcagagcagcctcagcggctcgcaaaccgccagcgacgatggtggtgagagcaaggataacagcgaggaacttcctgaaatggcacagaagaacgcgcaagatgctgcaattcgcggtctctatcgcgacattgctgctaaagatcgcctctacaagcgtcttagctccgtggttggtgcgttcgatcaccgagctatggactcggctgaagtcgctgtttacggcgtgaaaaagctgaacatcagctgtgcgaagggccaggaagctctggcgctcgacatgtacctgaaaggcgtcgaagcctcgcgcggcgcggccagccgtcaatcgaaagcccaggattcggccggttctgctccgcagtgcgccgagctggacagctacctgaagggggagtaactcatgttccagaaacaagtttaccgccagtacactcctggttttccgggcgatctgatcgaggacggcccgaagcgggcgcggccgggtcgaatcatgtctctgtctgccgtaaatccggctgccaccgccaccggccccaaccgcatcagtcgcgctttcggttacgccggtgacgtcagcgccctcggcgaaggtcagccgaagaccatcgcggctcgcgcttctgaagtcgtgatcggcggcgccaacttctttggtgtcctcggtcatccgaagcactatgcgctgttcggttcggccggagactccctggctcccagctatgatctgcccgatggcgccgaaggcgagttcttcgacatggccaccggcctggtcgtcgaaattttcaacggcgccgcaaccgccctggacctggactatggtgacctggtcgcctatgtaccaaacaacctgcctaccgccgacaacgcgcttggcctgccggccggcgccctggttggcttcaaggctggctccatgccgaccggcttggtccagattcccaacgcacgcatcgttaacgccatcagtctgccggcccagtcggcggggaatctggttgctggcgttaccatcgtccagctcacgcagtaaggaggcgtcatgagccagatcagcaagacccattcgcgcctcgcaggccgcaatgcgaaacctttcgacctgaaaaacatcaccaatgacgccgtggcgtctctgcgccgcatcggcctggtattcgatcacgccgtcgtccaggaccagatcaaggccttggcgaaggccggcgcgttccgctccggctcggccatggacagcaacttcaccgccccggtgaccacgccgtccatcccgactcccatccagttcctgcagacctggttgcctggcttcgtgaaagtcatgaccgcagcgcggaagatcgacgaaatcatcggcatcgataccgttggctcctgggacgatcaagagatcgttcaaggcatcgtggagccggccggcactgcggtggaatacggcgaccataccaacatcccgctgaccagctggaacgccaacttcgaacgccgcaccatcgttcgtggcgagctgggtatgatggtgggtaccctggaagagggtcgcgcctcggccatccgtctgaacagcgccgaaaccaagcgccaacaggcggccatcggtctggaaatcttccgcaacgccatcggtttctacggctggcagagcggcctgggcaaccgcacctatggtttcctgaacgatcccaacctgccggcattccagaccccgccgagccagggctggtccactgccgactgggcaggcatcatcggcgacatccgcgaggccgttcgccagctgcgtattcagagccaagatcagatcgatccgaaggcggaaaagatcaccctggccctggccaccagcaaggtggactacctgtcggtaaccacgccttacggcatttcggtttcggactggatcgaacagacctatccgaaaatgcggatcgtgtcggctccggaattgtccggcgtccagatgaaaaaccaagagccggaagatgctctggtgctcttcgtcgaagacgtgaacgcggccgtcgatggcagcaccgatggcggcagcgtgttcagccagctggtacagagcaagttcatcaccctgggtgtcgaaaagcgggcgaagtcgtacgtggaagacttctccaacggcaccgccggtgcgctgtgcaagcgtccgtgggctgtggtgcgctacctcggcatctaacagatgcctattcaccaaaggccgggcttccggcctttgttcactctgactctaactcggttgtaggggccggttagggcataattgctaggactgcgccaatgactgtttatatcgtttcctccatgactcaatcggtgtcttacaatgcgtatgacacctctgatccgtccaatcctcgcctccagcgaaagattctgattcgcggccgcgcaggcatcgcatccgaaacttccggcttcggcgacatgatttccgacgaggccggacgtccgatctggaccccacagggcgattgtactgccgtgagcgattcggatttcgagctgcttcaggccaataagattttcatgcgtcacatggagaagggttatctgcgagtcgtgaagacagacatcaccagtgaccaccagcggatttccaaagagactcgcaccatggagcgcgatggattccagcctctggacgctactcgcctgcagcagaaaatcaaggtgaccacggccagcgcttcccaggaacaagagttccggatttaaccgagggtttcggtatggtgattttcgacgaaaataagtttcgcacgctgtttccggagtttgctgatccagtcgcttatccggacgtgcgcctgcagatgtatttcgacattgcgtgcgaattcatttctgatcgcgattcgccataccggattctcaacggcaaagctctggaagcgtgcctgtatcttctgaccgcccacctcctgtcgctgtccacgatgcaagttcagggcgcgtctggaggtggcgtaacagcaggtgggacccaaggcggtttcatcaccagcgctacggtcggcgaggtcagcgtggccaagctctcgcccccggccaagaatggttggcagtggtggctttccgggacgccttatggccaagagctgtgggcgctcctcagcgtcaaagcggtgggcggattctacatcggcggtcttccagagcgccgaggcttccggaaggttggagggacgttctggtgatccctggcgcgaatcttctgcgaatggccttcggggtcattggcactcaaattgtgagatatcgcaagtttgagcagcgagtgaagaatgatcaagcccagtacgtatccatgttcggggagcctttcgacctggcagcgtctgtccagcgagtccgacgcgatcagtatgtccagtttaatctggagttccaacggaattatgttatgatcttcgccaactttgagatggttgacttggatcgcgatgtggccggtgaccagttcctctggaccggaagagtttttcagctggagtctcaaggctcctggttttatcaagacggctggggagtttgcctggccgtggatatcggtgcggccaagctcactgatgacgggaaaccgactttctaggtgatgtatgtttgacggcgaactgatagcgaaaatggttgtcgagctgaatgcggcgatgacatctgctcaagaggctttgcagttcccggattttgaagtcgtccagaaagctcagccgacccaacagggaacgtcaaccaggccgaccatctttttccagaaactgtttgacattcctcgcggctggcctgccaccgattggcacctggacaacacggctcgcaaatatgtagaaataactcgacagcatgtagagacgactttccagattagttcccttcattggcagaatcctgaaataactcacgtggttacggcttccgatatcgccaactatgtgagggcttatttccaagctcgatccacgattgagcgcgtaaaggaactggacttcctcattcttcgcgtgtctcaaatttccaacgaagcattcgagaacgacaatcaccagttcgaattccacccaagttttgacatggttgtaacttacaaccaatatattcgcctgtacgaaaacgcagcatattcggccgatggggtattaataggcatatgagtctgaggcgcgattcagagctaatcgccgcgcacctccagatgttaagagccatgcgcggcaggtccgtttcagccggatggtattccacggctcgatatcctgacaaggcaggcgggtcggtcggaatacaagtcgcgagaatcgcacgtctcaatgagtacggcggaactatcgaccatccgggcgggaccaggtatattagggacgccattgttcggggccggtttgttggcgttcggttcgtcagaaacgactttccgggagaaaccgaggtaactaagcctcacagaattaccatcccggctagaccgtttatgcgatatgcttggaacttgttttccgcagatcgcgccgcaatccagaaccggatagccatgagtctggccagaggacagatcactccagatcaagctcttgcccagatcggcctggcgttggaaggatacatagccagaagcatcaggaccgggccatgggtggctaactcagcatctacggtcaggagaaagggattcaacagaccgctggtcgatacggcgcacatgcttcaatcgattagcagcagagtaacataaccaggagatcatccagtgatcagtcagagccgttatatccggatcatttcgggcgtaggcgcaggcgctccggtcgcaggccgaaagctgattctgcgcgtcatgactaccaacaacgtcattccgcctggaatcgtcatcgagttcgacaacgccagcgcagtcttgtcatacttcggcgcgcagtcggaagagtatcagcgggctgcggcttatttcaagttcatcagtaaaagcgtgaattcgccgtccagcatcagcttcgctcgctgggtaaacaccgccatcgcgccgatggttgttggtgacaatctgccgaagaccatcgccgatttcgccggcttctcagcaggggttctgaccatcatggtcggcgcggcagaacagaacatcaccgccatcgatacgtccgccgcgacttctatggacaacgtggcgtcgatcatccagaccgaaatccgcaagaacgccgacccgcagctggcccaggctaccgttacctggaatcagaacaccaaccagttcaccttggtcggcgccaccatcggcaccggcgtcctggccgtggcgaaatctgccgatccccaggacatgtccactgccctcggctggtccacctccaacgtcgtcaacgtcgcaggccaagctgccgatcttcccgacgcggccgttgccaagagcaccaatgtcagtaacaacttcggttcgttcctgtttgccggcgcgtcgctcgacaatgaccagatcaaggccgtgtcggcctggaacgctgctcagaacaaccagttcatctacacggtcgcaacttccctggcgaacctcggcactcttttcaccttggtgaatggcaacgccgggaccgccctgaacgtgctgtcggcgactgctgccaacgacttcgtggagcagtgccccagcgagattctggccgccaccaactacgatgagccgggtgcttcgcaaaactacatgtactaccaattccctggccgcaacatcaccgtttccgacgataccgttgcgaacaccgtcgacaagagccggggcaactacatcggcgtcacccaggccaatggccagcaactcgcgttctaccagcgcggcattctgtgcggcggtccgaccgatgctgtggacatgaacgtctatgccaacgaaatctggctgaagtcggctatcgctcaagcgctcctggacttgttcctgaacgtcaatgcggttccggcgagcagcactggcgaggcgatgaccctggcggtgctgcagccggttctggacaaggcgaccgccaacggcacgttcacctacggcaaggaaatcagcgccgtccagcagcagtacatcacccaagtcaccggtgatcgccgcgcctggcgtcaagtccaaaccctgggttactggatcaacatcaccttctccagctataccaacagcaacacaggcttgaccgagtggaaggccaattacacgctgatctattcgaagggcgatgccatccgcttcgtcgaaggatcggatgtgatgatctaatggtttgcggcggactcgatccgccgcgaccttccataaatggagtgaggaataaacaatgatcaacatttctgcgttcggctcgatctgccagttcacggcaagcagaactttcccgaacggattcaccgtcaccgagtttgccgacgatgcggaccccatcgacagcccgccgttcactgcggccgataccggcgtcggcctcaacggtgacatggtagtctggaaccgggcgaacatcctggaagtcgtcgtcaacgtcatcccgaacaccgagggcgagcgcaacctggcagtcctgctggatgccaaccgcaccgggaaagacaagtcgggcgctcgtgatgtcgtcggtttggtcgtggcgatgccggacggcagcaaaatcacctgcaccaacggcacccccatcgacggcgttctgatcaacgcggtggcaagcgtcggccgtctgaagaccaagccgtatcggttccgattcgaaaaagtgatcaaagccggtactagctgatgaagaaaattccgctgacagcagtccccaaccaggcgatctcattcaacgccggtagcagctattggaagattcgcctgtaccagaacatggacatgatgaatgccgatatcagccgcgacggcgtgatcgtttgccatggggtccgctgcttcggcgggattcctcttctccaatacagccatcagtaccgacctgactatggaaatttcgtcttcgaccgcgacgccgattggacattgttcggcgacggcatcaacctgttctatctggacggcgccgaattcgccgagtatcaggcgcttgccacgaggaaagaatgagcacatcaacgatcagaaccgggacgaacaacgatatccttttggacgacaatggaaacatggttatcctcagggatgtcgaagcgtgcgcccaggacgttcgggcggcgatgctcatgcgcaccggcgaaaacattttcgatgtgaactccggcgtgggatatttcgaatacatcttctcgccgcagaaaagctatgatgacgctcgcaaatccatcgcggatgcaattttatcctcgccggatgtgaccggcatcgagcagcttgacatcgacataaccggggaagtcttcggcgtcgatgcgaaagtcatcaccatccacgggcctgttaccacaggagtttgaaatgagtaccatccgcatccaatacgccaacggcacccaactgttcctggacggcaaaaatccgccgcccctggacccgctgccctcgtttaacccgtctgtcgaagatctggaaggcctggaccgcgaaaagaacatcgacaagggcgactcctcgccggccggtcttcccgttcccccggtaaacgtcgattccaatgtcgacaacggcggaaccatcccggctccggtatcgaccgacgctgctgcagccgaatcggccccggaaggcgcccaggaagctcctgcagcaggccaaggcgacgagaaaggcgccgaggaagccccgactacagcccctgtagaaaaggccgaggaaacggcctcgccggccgctgaagaggaaaccccggctcccgccaaggccacctctcgcaaaaccaccagcaagtaaggactcgacatgatcaacgtcagcggcttcggcacaggaattgtaatagtttcagcctcatcgttcccgatggggttttccttgtcgaagttcgctgatgatgagagtccgatatcctccaaagagctggagccgttcggatatgagatgctttatgacggcggcctattcgcctttgacaaggctgctcctctggaagtatctgtatccgtcatcgcagggagcgaggatgatattaaccttcgcatccttctcaattccaaaaagggatcattcagatttcttccgggcatcatcccagatatgacgactctcgtggccactcttcctgatggcggccgcaccgttctgtccaatgggactatcatcaagggtccggccatcgacaccatacagaacaccggacgacgcaaaggcaacacgtacacttttgttttcggtagctatcttggcgcccagactgcgcgtcaagccatttctaacgttatccaatcggtactggaggtggtctgatgttggggattttcaccagcctcctaagttcgcgatctttttcgattgtggaccaaaacacaaaccagctagttgctgcggatttgaggataagccgggtcaacacccggttttcttctgtagggcaacgccacatgctggaagatggtacgaccaagatggattccagaacgatccaccctatggaaatcatcgtcgaagtattttgcccttcaattgatgtcgtcgatcagatcaatcaattgctcctggatcgcgacacactgtacaaagtcatcactcgcggcatggtatttgaacggatgatgtgtaccagcgaagcgctcaatcagactccggatatgatatcggcgactcctgcgcgcctgacattctcccaagttctcgtccagaatcccaagcctataatgttcagaaatgcaggggactcttctatgatcgaccgagggctggccctagctgaagacgtggttggctcggccggcgatctgttcgactacgcagtaaacggcgtccagaacgccgcagacttgttctgaggtgccaattgaactctttcctcaagtctattctcaacacgcctactctcaccatacgcgatgatgttaccaaacttcctgtctggaagagtcttcaagtcaagaaagtggaaatttactcgccggcttccgtagtgtcgaaacctctggcgacgaaagaccagacggaagctcaagtgtacaccgaagctctggacattgatgtgaagaatggaaagatcatccaaccggtgcgactccgcatcaatgctatctgtccggacctgtccaccgttgaaagtatcatgaacgctttcaatgataacacctcgactttcgctatcacttctaagtcgatattggccgataaaatggccatcatgacgctcgatgtagatcagtctccagacatgttgaacgcggctgagatcaatatggaattcgagcaggttgagcctccagtattgaatgaattcgatcctgcgttccctcaagatagtccgacttatggggtgcagattcaatctctttctgatgctaatttgctggatttgggcgccatcggcgattcgatatcttcggccgcaaaatcgctatataatcgcgtgaccagctacttctgaggatgtatcatgcttgaaatcaatcttcccgatggccgtcaaactcgcgtacaaatcgaggcgtggtcggcattggatggctgggaactccagcgccgtttcgtcgaattcgctgtcagccaagattccgacttccgccgctctttcaccatggaaatcctgggctatgcgaaagtgctgcttggcgacgatgacaccggcattccgctgaccaccgcggcagtcatcaacaaccacctcggccactggaagaacgtggaactggttttcaactctgttctcaagcacaatggcatcgacccggccacgcacgccgaccggccggactattgggaacaagtcggatcgcagatggccatcgcatttctggccgaggcgtccaagctcattggtccagcaatgaagatcgccgaaggactcgccaacaagccggagtgattcatgtctagtgatttggatgaattcatacttcggtatgaggccgatactgccagagccgagcgcaatctggagcgcctccagaaccagatcaggcgcgtgaacagcgcatcgacgagtggccttcaggatttgcgccacttcgcagacggcgcggccaccgaactcggccgagtcgttccgcagattgattctgtgacgagcgcaattcgcggaatgaacgcgcaactggcaataggcgccactggcgtggccctggtcgcggccggcgtcaaggcgttcatgaacaccagggaccagtacaatcagcagcgcatccaggcgatggacatcggcatcgccccggcacgcttggaagagtaccagagaaaattcgttcgccagtccggtggaaccatcagccgcgagcagggcgcggagatgaccaaaaatctggcagacactttccggcgtgcttatcgcgatatcgggcgagtcggcccggaagcgcgaattctgcgtatggcaggcgtcgatgtcgggagcttccaaaagggcatgcggccgctcaacgacatcattactgatctggccacgaaaatggccaagttgaagccggacgaaatttctgcctacgctgatgcccttggcgtctcgcgagactacctgagcaccctggcgaagatcggcccggccatgggaaaagtcactgagatgacgtcagaagagcttcaggctagggtcaggggcgagtcaaacattcagaagttcaatgatgctttggcaaacctcaaccaaacgttcacgactctggaaaaccgcgttggcgaaaaactcgcgcctgcattcaccaagttgatcgaaatcatcgacaaaattgtccaggccattcccaatgaagtggaagaattcgccaaggacacgaaagcccgctgggacgatggaatcaccggaaaggctactgtgggcggcgatatcctgtcccttctcagccctggtgctctgctaggtcgtctggcttcctggggcactcggcgcggcatggaagaggccggattaatcgacaagtcaaaggtcccaggctcccaaggccaaaccagcgaagacctggccaagaaacaggaagaccaggacaaagctacgaagtccctgaaagagctggagaaattggccgaccagactacgaagtcaacgaatgatttcgcggtggcgatcaacatgttcagcggagccgtgtcatcgttcgccaatgccgttgacgagcgtcaagcatgggcagcatgggcgggggaaatcgggcgcgcagttggcatgggaagcaccgcgccgacttcgcgagcaacaggggtttatccgcacgcgatctacgatcagtcgaagagtggcgcggccggacaagtattcggcgagcctattggcgcccagtctctgcgaaacaggatgttctcgccgcagcgcaaggccgagccgataaatgtcccatcgtacatcaatgatatcatcaaagatgcgtctaagatgtacaacattcctgagctggacatcaagaagctcatatacacagaaagccgattcaacgctagggcgaccagtgaagccggggcgaaaggcctcatgcagctgatgccggaaattgccaaggcgtatggaatcaccgatgtgtatgacccacgccaaaacatcctcggtggaacgcgcctattgcgggaaaacctggaccgggccaaaggcgacatgcgattggcgttgacctactaccatggcggcctcgacccgaagaactggggaccaaggactcgcgcatatcctggtttggtgatgagcgcaccaattgaactgatggaggaagcccagcgcaagcagaaggctgcggccatgacggtcgccaacgagacgttcgcgccagaaggtggcgacatggacattcgcccctatgacggcggaaggctggaagctccggaccagggcaggaaggaggatgatcgccgcgaagctcgtcgatatgacgacagagttgtccggccggagattcgcatcatcgaccgcatgccagaccgcagtgacggcgaaattcttaaaatgtctcagcgccaagacgccgaccgggcggactctggattccggaaattcccgaaccaggttcgtggcgagacaaagcagaacatccaggcccaactcactgccggagctattgcccaagtcatcggtgttaatcctaaccaaattatgcgccgcgaaatcagccgttccgacttgctgttcggatacaaccaggccatcttgggcaaacagcaggaaatcaaagccgctgcgacagaggccaacaatgtattcctctctccagccaaactcgctgaggctactgccaaggtgaacgccgcatcgcgagaaatggatattctcaggacgtatggtgagaaacttctgaagagcgctccagagcgcggccaggaactgacaatcggtcgaattgatatgttggtcaacgtcaccggcgcgaattctccagaagaggctcgcgaaatcttcagcaggcaaaccgcagaacagctgactactgccatccaggactcccaaaacgattccgcaactaagatactctactgatgaaaaagagaattctgcgagtcacgttcaatatgccctatggacccgaaatcatccgtgaagacctggatgttcgggtacggattatgaaggctgcattgcgaattcaaaaccgagctaccctggaaatctttggcctcacgacgcaattgcgcgagtctcttctgtcgcagttcacagcgtggaagcaccggcagcgccaagtcggaatggaagacgagctgatgatcagagtatcggttgaggccggctattccgatcagggtcgcgaacaagtttccagagtatttgtcggcgaagtggcaattgtcgatgtcatttcgccgccaccggatattggaattcgcatccaatgctacacaaggcaaatcgataggacgaagactattcgaaatatgccgccagccaacacgacgtttgtaaagttcgtcgaatggggcgcaaatgaaatggggcttaacttcatctgcgataccagttacaatgatcaagttttgaagaatccgggccggtcgatcactgtcgcgtcggcaatcctggcatcgattcaggatatgtacatgccggatgtggccgcgttcgtcgatgatgacattctggtcgtgaaggaccgggataaggtcattcgtcctgatgaagttgccaacatcaactcattcgtcggcatcccttcatggtcggaatggggcgtggaatttcagtgcctgtttgaaccgtcgattcgcgtggctggcggtgtcgcggtcgaatctctcatgaatccaagcgtcaacggcaactatgtgatcaccgctctagagtatgatttggccagccgggatcggccgttctatatcaaagtcatggggagcccagcagcgtaatggccagggaaatcaaatcattcaatatgttcggcgtgcactacaactcgcggcaattctctgcggtcgatggactcaggatgatgtcgggaatccatgatgttcctccggaagaattgctcaaagggaccgacgtgttggcccatacggaggaacaaccggaaggcgtttggcttcccttgaccgctgcgaacataaatctttatgtaattgaccgggcgaacgtaatagctcccgtacaagtgcttgcgcttttgtctgaactggtcatagattggaactttggcttcctcaaagattggacaggggtcaaaattccatcaagatttgtagaagatatcaaaagcgtgaagacggcccattcgccttccgtggtcgcaagtttggtggcgaatgggtcagcttctatgcgcgagctggaagagtattattcgactcaagatgcctttaagatgattgacatcatgactgcgaagagcgtgaatgaggctctagcgtccgaagcatcacagaacagaatcaaaaagggataattcctaagcgagcctgggaaggctatactagaccggccaaatcagaggctttcccatgtccaatattccgctaacatccgcaaaatctaccgacagaacgcgactgatcgccgctcttgacgctcggtcgcggcgggatgcgctcgactttgaagtcatgattcccgcccaggttgttcaatatgatcgggcagaaaacatcgctaccattcaacctctcatcacctgggttgatacggaacacaatgccgtccagcggcatcagctggttgacatcccggtgatttccatgggcgctggcggcttccacataagtttcccgatccagcagggggatatcggctggatttacgcggccgaccgcgatacttcccagttcttggagtcgctatcgatgtcgaagccgaacaccggccgcatccacaaatttgaacatggtatgttcataccggacgtattccgccgatacaccatcaattctgaagactcggacgcgatggtcatccaatctactagtggagcgaccaggatttccattcgcggcgacaacatcaagatcactgcgccgtcgaatgtaacagtggatactccgcaggcgaatttcactggaaatgtgactatcgccaacaccctggttgtaaacggcgtcaacgtgaacaaccacggccacctcgaaaacaatccgcctgatgcccgtactaaaggcggcatgattgcctaaggagaatttaatggctagttttgatttttctgatttaacagcgggggggggttgtaatggctaattatgactacatagtagatactggagtcatagtcgccgatactgctgatattctgaaggacgttgaagcggaattcagggcagccctcggcgccaatatcaacctggcggcctcaacgccccagggaactctggtcgcggctgaaaccattgcgcgttctagcgtgatgaggaatgaagctcgcatcgccaataccatcaacccaaacgtgtctttcggaacgttcctggacgccatctgtgcgctgatgggaatcgagcgcggctctgatctttcgacgttcggctatggcgtccaggtgaccggccgcagccagacccgaatttccaccgggtcgcgtgtgcagactccggccggagcgattttcacggtcatgagtgacgttctgattccggcaaccggagtcgccaccatcgacgtaaaatcgcaggactatggaaacatccctcttcccgtaggaaatcttatcatcatcgatggaaccatcggttgggccggggcgaaagtcatcgcttcaactcgcgtcgatcctggcagccgccaaatgaccgatgcagagttgaagaatgctcgcgtcaatcgtctggcgatccaaggccgcaactcgactttggccattaaagcgtatgtcagcgccgtgcccaacgttacctcggtcaacgtcatcgaaaacaacaccggcactgttcaagttgtcaacggcgtatcattcacccttccgtatgcggtctgggtctgcgtcgccggaaatccggataagcaggctgtcgcagatgctctgtgggcggcccacaacggcgggactccctgggactatggcgcggccgacaacggcgtccctgtggatgggcctactggcgttcctgtgcgcgatccggcgtctggtcgaaagtatgtcgtgaagtggaccactccaattatgtacgatggatatgttaacgtaacagtccagcaaggttcctcctctgttgctccggaagccattcagaacgcagtggtcaattacgcccaggggaaagtggaaggggaagaggggctggtggtgggcgcgagcctgtcggcctttgaagtggccggggccatcgctcgcgagattcccggaatctacattaaactatgccaggtggcttgcgtcccggctggatcgccggccccggcccccggcgacttctcgcctgagtacgtcatgagcgcattcggtcaggcaaccatttcggttggcaacgttagggtgactttcgtatgactctgcccgcgtacaattctgatattcaacaggcgctgaagtggctccataaccaggcccctgggatcaccggcttggttcagcgaaaatctcaatggtatgaccgtttcagtcgtcagttttgggtcaactgggagcgcgacgttttcaacctgaagaccgccaacccgttcggcctcatggtgtggtgcatcatcctcggcacgccgtcgaaaggattcggcctatatccaaaaaacagttcttgggcattcggtcggctacgccagaacttcatctatagcggtacacaagttccgccaccggcagacgcatcgccgggcggcaacttctacggtggcggcaatgccgaaattctcaacttggacgaaatcaggaaagtgcttcagttaagatatgtagcgctgatttcgaacggctcgattgcatatatcaatcgcatgcttcgctacatattcaatgatgatgagccgtgggacgaggcgaccggtctgtacttctatctcatggactcaaccggcgaggatggccctgtggagaacttggccatatatcggaaagattgggaaggtatggtgctgttgtccagttcgcccagaacgaaccatgtgctgacatcgacccctgccagcgacgccgattggccgggagtcgatccggccgcgagcggtcttccggtaacggtcgaaacggcgtccgctacggccccggacggctccgctacggtgtgcaagcttactaagccggccgggagtaccgcttacgtctccgcgccgatagatgggccgctggggtccggtagcactgtaacgttctcgttcttcgcgaaagccggctccacccgtttcattgcaattcagtcggctgccgatttccccagtcgagccgatgccgttttcgacctggattccgggcacgtgatcagcgatcagatgttggacagcagcgtggtaagcgcccgaatgattcgtctggagaatggctggtggcgttgcgttctcacgaccaagaccgtcagctcttcgttccgcgcggcttacatcgctccggcagaaaccaacttcagctggattgattcgaattccagcgcggcgattgatgtgcttatctggggcgctcagatcgaactgggtgatactccaaccggatacttggagactaccggaacgcccgtaaccatcaccgattacgttttgcagagcgcccagaccggaacggtcaagttcacacagcctcttccgaccggagtagaggcgtattggactggagactggaaaggtgggtctgcgaccgagccggccagattcgcagcaggggatgggactcaagatacattcaatctgtccagccctgcatacatcggcctacccactagtggggcgttcaagctagaatacagagttggtccggcgcttaatttgtcgccgcaattgatcaacctcatgaatgaccgggcggtcggtatcatgccgacttgcgccggttgcgatgtaaaagtcattcaggagtaatgacgtgatcacacccgaactgatacccagtccgtttgctgcgcagggcgacaaagacccgatcccgcagacctcttccactggctttgccaaccttcgcgacggctacacgccggactacgaaatcagtctggcgtcgaacaaaccgcaggccaaagcggtcgagcgaaaaattcaaaaccaactcttcttcatcgcgacccagaacgcacaggcgtggcagcggcaaatggcgccgccgtggtttcagggcatgcctggcggctacgaacagaatgcagaagtcgtgcgagtcggcaatgacggcataatgcggcgttatcgttccatggtgaatgccaacgcgagcgaccctctcagcagcacgacttgggaagaacaacccgcctggtcggtgatgcgctccaacataccgatgcccgctggagggccaggcctatcttctggcggagaagtcatcacgaccggccgcaacttcaatgacctgttgaatgggacgtgggagttcttctctgattcagtggtcgtcgcttctcagaacgcccccgtatatcccgcttcggctggtgcagcagctggaatgttggaggcgaaatcctggatatccgggtccaatacattctgcgttcaacgctacactgaccgcgtcgggaacgtcgctgtgcgcgggcttaatgccggggcctggaccaactggatgtacgcagtaaatgtcatggccctccaacaaggccgtgtgacctatggagtcgcggccggatcggcgaacgcttacacgttgacgctagttccgcagctccaaggcggcctggtggatggcatgattcttcgggtcaagttcaacaccatgaacaccggcgctactaccatcaacgtctccggactcggcgccaaagccatcgtcggcgcggccaacttccctctcactggcggcgaacttggccaaggactcatcgctgagcttgtcttcgacgcagcaggcgaccgctggaggattctggcaggcgcgccgcgcatacaagtcggcaacgccgatcaggactaccaggcgccgagttggaagcaagttaaagactatgtcgcgtcccaaaagctcaccgaagtggattggacggatgtcgtcaacaagccgaacgtcgctatccaagatacaacgccgtggttcgccaatctggagctgtccgatgctcggcctttcatcgatttccacttcaacagcaatcgcgccaaagattttgactatcggctgatatctgaagcagacggatcgctggctttctattcgcggcaggggtctgctgggcctacccaggacatcctgttcaaccgaaattccgtgactttcttccagccgcgactcgatgtggcgaaaaacctcgcgtacatcgcgaactctggccccctttggcagaacaccaccgccgatcagcccggttggaaattcacctttgcacaaggcgtggacgcgaacaacaacgcggtgatcgcagtcaataccaccaatccggacggttcctatcgttcacaggtcatgcgatgggactgggcgtccacgaacgtcatattcaacaaccgtccgctcttcgccggtcaatacaccccttgggattctgggaacttcgatccttccaccaagttgacggtgaatgccacgaaccaaatcgccggcccaaccgggattcggaataccaacggcaacaccggcaacatgaacacctggggttccggctccacgacggcatcctatggcaatgctgccattcaaatcttcggaaaagggggcggtgagcctgccgcgatctatttcgacaactcccagaccggatggtatctgggcatggacaaggatggacagctcaagcgggccggctggtcgctcggcaataactcctatgtgatcaccgacgaatccaacatccaaacccatatcaacacaatggctgcgcgcccgatttgggggaatgtcgagttctggggtccgtggaacttcaatccgaacactaagcttacgttggacgcattcaacgatagccaatacaccaggatggtaaacagcgccgcgaaggacgtagggatagccgccatgacaagttatggcgacgcggctatgtcgttcttcaactatgaggcttcgacgccgaccgggaatcgtgctgctgtaatttcgtttgttcgtaacggggcacgaggcgttctgttcggcctggacacggacaacaagctgaaatggggcggctattctctaggtgccgtcgcgttcgagattgccgactccaacaacctcatgagcctgtggtcatcccacgctgccgcgccgaactggaacgggcagaccatctggaggtcgggaaacttcaacccagacaccaaggcgactttggcagctcgcaatacgacgtcatcccctacaatattcagttatggggcgtccggaatcgcatcaaccggacaggtcggtgcgttggttgtggaaaacaacagcgtcaccaataccgcagccgccatcacgttccattcgccgcagaaatatcaggtcaacttcggcctggatgcggacaacgtggtaaagatcggtggcggcacaatgggcggcgtagcatatcccatcatccactccggcaactacaacaactacatcaaccaggcgctggttcaggtaggtcttggcggcgtcggttcctacgcgatccttgccgtattggacacatccgcgccggcagcgtccattgcccccggaacgatcatggacagttccaagctgttctattcgtcctgcgattcgacctatcgcagcagcgcccgtccgacgggcacctggcgctgcatggggtatgtgtataaccgagactccaccaacggcgactcggcatccctattccagcgggtaacgtaacatgaaatggacgcggatcagaaacccacgttggctggacgcggtaaacatccacgccatggttactttcgaggggattggagaagttcctttcaccgccaatccgcacgacgtggaggcccacggaagggccattcacgctgcgatcctgtccggggcgcacggccctatcgccccagtagactcgacgcgggagcaggccttgcaggacgctatacgagacagggaaaagcgggctatccttcgcgatacccgatggcccatagatcgtcacgacgagcagaggcggctgggtatcgaaaccacggacgggcctgggctgatagcagcccttgttcactggaggcagcagattcgcgattggaatagcggggatcggccgcgacttcccatggctctgaaaacaatgttcaaaaatcaggagtattgatgaaaatcacgaaggatgttctgatcaccggaaccgggtgcaccacggatcgggcgatcaagtggctggatgatgtacaggcggccatggacaagttccacatcgagtcaccgcgagccatcgcggcctacctcgccaacatcggcgtcgagtccggcggactggtaagtctggtggagaatctcaactacagcgcccaagggttggccaacacctggccgcgccggtacgcagtagacccgcgagtccgcccgtatgtcccgaatgctctggcgaaccgcctggcccgtaacccggttgccatcgccaacaacgtgtacgcggatcgtatgggtaatggctgtgagcaggatggtgacggttggaagtatcgcgggcgcggcctgatccagctgaccgggaagtcgaactattcactgttcgccgaagactccggcatggacgttctggagaagccggagcttctggaaactccggccggcgcgtcgatgtcttctgcctggttcttctggcgcaatcgctgcatccccatggcggagtccaacaacttctccatggtcgttaaaaccatcaatggcgccgcgccgaacgatgcgaaccacggccagctccggatcaaccgctatatgaagaccatcgccgcgatcaatcaaggctcctgatattccacccaaagaaaaggccgcttattcagcggcctttttgctttctggctttgcctcttcaaccatcttgacttcaaccggcgcggcggactcttcctgagtgaccgaatccacatagttccctagtgaactcagaacgccgattaacagcgctcttaccaccttgtccttgactgtctcgcctatgatcttggtcagaacggatattaactcttcccggagtctggggctgattcttggccgaaagcgcttgcgatgctctttgcgtttcatgtttagtcctctgtctgcggtcttctcctcaccccgataatggcttggggatgcgctgtgttaatcggaaggttcgggcgctattataactcgtcgaaaatgctcgcgcttaactgtttaacgatacgcaccgcgatattaaatcgccttctttctggccaaggaactctggcggcctggtccggtctaaggcctaatttgtcgacattaaaacgagaaaacccggatcgcctgtaggacaaggcgtccgggtttatctcgatctagtgtacgctagaatcagtggcttccgccccatccgtccagccagcaatcgaagacggcgtgtcgcggcttgtccttggcgccatgagagaaatgcttgaatcggatgacctggcccttgatggactcccggtcgttccagaaacgacgtttctcgtcgtgggtcaggctggatgccgacacgttgaaagtgactccaggccacagacgttcgttgcggcagactaacgcaccgaccattccggacggcgacaggttttctgcgtggctggagcgggccgtgtggccaagttcattgatgaaagcttcattgttgttgtgcatcaactcttccacgtcgatgatctcagcttcatcgtagtcgtatcgcttgactttcacacagtgcccttccttggcagtcgagcgaccgaacttgtacagcccatcagcgcgcttgcccatggaaccttcgaatcccagcattgtgtggcggcgctcgacttcgctgaactgttcgatggaagtcaccagttcctgctcgaccaggtgaatcctctcatagccgaggcagtgacgaaggaagttcacgcgctcggccgccctggctaggcgatcttcggtcggtgcgcgaggatcggtgaaatcatcgaacacatggaaagaccagtccggctcaccgctgtggcggcgaagatcgccggacgacttctggaaaactttcgggtcgctgatgtcgccgcagaccagctcgccgtccaggccgttgaacaatttatcgctgaggtattcgtagatcgattgattgttctgccgcttgagttgacgagtcagcgcctcgccttcgaatacgaagcagcgaaatccatcgatcttcggcgagaaatacatcggcagttggccttccagcagcttcgggtcaaaattcgatgcgagcatgggtttcatacagttctccagaaaagaagcccggcgaaccgggctaaatggcggtaagccggctcagatggtttcgttggcatagttcagttcggccatgatcgacgcatagcgctcgtccgactccttgatgaaaacgccgtcgtacatgacgcctttgcgatccttgatggtgtcgtaggctgcagcgtagcagtcgagcatggtggtgtcgtgctcttccgtcaagtcgaacagagtcatggcggccgataccaggctcttgatggccaacccatgatttccacgtgccagagcgcctgccagatcgccgaggcgctgcaaatcgtcgccgtaggtgggatgtacttcgccgctctcgatgaaggccgacagatgatcgaacagattttcgccgagttgcgcggccatgatcgtggcaaccaccattacgtcgccgatgccatctttcacatcgaccgggttgttctgaacccaggcttcgcatacttctgcgaactcttcgaccaacttcaggaattgatctttcgcagacgagcctttgatcaggttacggtcggaagcccatttaacaaccaggtcgtgaagttcgcgactcatgatttgttcgatattcattctttcgattccttttggatttgggattttactgcgttgattatggacgccgtgctctggcgcgatccgtccttggtggtgccgaagtagaaggccattaccgacttcagctcggcgaaccaatagccgatgatggtgccgatggcgacagaggatgtcgggtccatcagagcctcgcggccgaatgtgaagattgcgatgatgatgagaatggagcctgtcagaagagcgaatgtaatcgccggacgaacgaaatcgttctgctgcgccgcaagcctcctcgcagaatccctgtcggccgcctctgcggcgaactggctaagctcggcctggagctggttctgctcagactgaaggcgattttgttcggcttgaatcgcaagctcctggagacgaacgcgctcggcgctctggagttcggcgagacgcgctagagcttccggattcgcgtctagagcgctagcgaccgaggctgggtcggccttcgacccaagcgccgtcgcgacgatagcgcccacggcggcgcctgcaggcccacccaggagcgaacccagagccggggcagccgcgccgatcttactacctatgtctttccagtccatttcgactcctcaaaagaaaggcgccattacagcgcctttctccggccggtgatttagaactcttcggcttcggtcgcatcgccgacgccaccggcgtcactgcgaggctgttcctgcttgctgtagtccaccttcacctcgccgccgacgaacgacttgtacaggtcggccgcagccttgaagtgatccgggttcttcaccaggccttccagctcgaactggacgccggaccagctacccttgtcgttcgacatgcctacggtggtcatgcggaccaggttggcgaaagtcggcggggtgcgcaggccctgcggggtctgaacctttttctgggacagcgcggtcatgagcttcttcgaggccttgatctgcgaagatgacagggagatcagagcctggccgaaatcgccggtgtccggatcaatgacgatgacgtaatgaccacgggtgtcggcgaagtagtcagacttcttgtcgctgaccgaaccgtcttcgttcggcgcgtacaggcgtccttcgacttccttcaccttggtcgggtctttcatcatttccttgaagtcttcgacgctgatggcgcccttgaaaccgccctcggcctcgcgaccggcccagcggatgaactcgcggcggtacgcggccggaatgatcagcaggccggtcttgccgtcgtaaatcttgccggtgacggtgttcaggaacatgccggccttcgcgccttcgatgtacttcgggtcatcttcatcgacctggggagacatcttctgcagaacctggatgaaggggatggcataggactcggcatcggcgccttcgaagcctgcgccgtcatacgatcccaaatccatgaagtcgggaacttcggtagttgcgacggcgccgccaccggtggcaactgctacggccttggtttcttcggttgcttcggaagtttcggttttcttaccagccatgttaggctccttgtttgtcgaatttcagttatcgctaactgtgggtttataataacggaagttgcaactaagtaaagcaaattacatattaagatttgctctttttcaccttcggtttcgtgattttggcctctttgtattcgtgcacgccgatgaaatctggcagctcttcgcccttctccaaatactcgcgaccgaacgcctggagggtctggtagtggacatcgcggttgatggtggcgtcatagccggcctcaatgatagcctcggccgccttcttcgcatcttccatttcgccgcgaccgaattcggccagaaccttggtcttgatgatgccgtcgttgtcggtttcttccagccacttccagaacttcgatttgttctcttctttgacggaaatgatggccttcggctctacttttaccgtgcgaccgtccgccagagtagtggtcttctgaccaagctcctccaacagctccggaatggtgttacgcttgagggtcttcagctcctcttccttttcggccagcgccttttgaagatcgaggatttcgccgtccagctgcgaagccttgtccaccaagttcagcagtcgatggccgatgtcggtggcttcaaccgccatttcatccatgacgccaaaatagtcgatttcgcccggcgcgttctctttcaagtattccgggatttcaagctcttgctctttcatgtccgcctccaacttagtgatgttcccttacttgaactaagtattgagtagatattatgccgcatcttccttgatacggctactgatttacatattaaatttcgtcgcgagtgctaacgtcagcctcaaacacgccatcaacgacataacttgccaggttgcgcttccactccaagctgacctggattttctcgtcgatggaatccagacagatcaggtcgaagtacaaaaccgagttgacggtgccgattcggtgatttcggtcttcggactgcatccgcaactcgttgtcttcgtcggtcgtgtagtaaatggccacgtctgcggcggtgagcgtgatcccgatccctgctgcggccgggttgcccaggaacacctggacgcgcttggcctggaaatcgtcgatcaatttttcccgctcggcctctttggtctcgccgtagtatgtgccaaacgaaattccttgggcctccagatacgccttgatctggtcgatttcctgaatgcgcatggcccagatgatgatggagcgctccgggtcttcctccagcagaccctccagaagatcagtaaacacggcgaatcgcgggttgtcttcgggcggcaggatcaccggctcaccatagacgttaatatagccggacgcaacttgcttgagcttcgaacgcgctgccgcagcatcgaaagatacatccagcatgaaatcttcgttctggagaacgaagtgatagtcttcttccacgcgctgataaacctttcgttgctccggcgacatttcgaagtatatgcgcttgtacaccttgtccggcaggaatgggagcgcttccttcttcgtgacccggaaactgtgcggctcgatcagggaccgaagcttgtccaggttccggaataccgggcgcccacactcgtctttttcgaccagttgcggcggcatggtctgcttcccttccagcttgcgcatgatagctatcattcgaggatcgtcactgggcaccaagacggaaaattcggccacaaacgcccgataggatttcgtccccagaattccattgcgcaagaattggaattgcatgaacaaatcggtcggtgcccgcgtcagaggagttccggaaaggatgcgacgggccacggccttctcgcccagctttacgatctttttcgcacgcttggcttgtgggtttttgatcctcgttgactcgtccacaattgcgcaaactttgaatgtatcaaggaatcgctcgacctcatcatagccagcctggtggttgattgcgtcaacgtttatggcgaagacgcgaagaactttttcatctgcgaacgtctcggcatacagacgatccaagcgcgctctggcctttttggaagtcggtcggccgcgccaatccacgctcaaagtcttgatcgcgacgtgggtgggaatctcgcgcaaaatccagttagtatgtacgcctttgggggcgacgatgagcagcgcgtcaacccttccttgcaggaagagtctaactgagtctgccaaagtagtccaggtcttcccggtgccctgctccatcaggtatgcgaaattccttttgttgagggaagcttccagggcattgaactggtgttgcatcgcctcggtcttcatacccttgactggaaaggttttggctttcatttgttctccagatcggcgagaaattgaatgatgttgtccagtccttctgcatgactcgcgacttccacaaggtcgcggctgttaagatcgaacagatcgagcatgggattcaggagcaaccaatcggttccgatcttcaccagaacgaagccgcgaccaccccagccgatccgctcccgaaggaaagggatttgcccaggctcgaaacagcgcgccattgggcaggtcgagttgcgctttggccaagcttccagagccttgaactcgacccagaattggacgccgtgacgattcagacagatcgaatctgacatgccggaccggcgcgtctccaggaaatcgaccaggattctgcctagcgagcgttgcttaaacgcattcgcggctttcgtttcgcgatcattcatcgccattcccctcttcggaatctttctctgcttgcgctgccaactttgctttctcgcgttcggtcaatatccgcttgacggccttcacgatgaacatatcgattccgctgagcttccatcctttgatgaggaaccaagagccggtcggcgtcccttcggcaatattctttccgtaccgaagatatttttcaggacgaattctgaaacgaatcggttggtcaaccgagtcgtccacgcacattaagtcgaggaactgcgactggcctttatacaccggattctttccttggtcagccctcttcttctggcggatcggttcattctcatccgacagaactttcttcaccagctttacgataactaggccatcgtcgccatcgcggatatcccgaatgttttgaatgggatttccggaagttaccccaaccaactcaggattgtcataggcatgaccccagagtgtatgagattcgttcaaatccgcgaattgaacctcggaatttgacaaactcgcggcaactttctcccaatcctgaagtgttttaagatgggagccggccagctctttgtattgagccttcaactccttcagttcagctttcaactccttgagatcagctttgagtagcttctccagttctttgtctcggctgactttcgccgaaagaatctgggcttccagcgcggccacgtcatcagccttatcctctacatcctgcgccgaaatcgggcaattggccagggcaatcctcgcggccttgacttcctcgcgaagacgcaagaatcgctcggccttagccgggccgaagcctttggcgttcatgatgccgccgatcaggcgtccgtccgctaccacccagttgagttcggaatgctccgggtccagggccgtatattctacgccttctttggccaattcgcgaaggatagacacagtttgctggtcgtccttcgccgcccggaggcacgcggccgcgtattccaggcgatgataccgcttcatgtagcaagtccagtatgtcaccacggcatagcttaccgagtgggagcggttgaatccccaggcgccgaacgtcaccatttcctgccaaactcggtgggcgtcttccggggcgactcctatggtcttggcaccttcgatgaacagttctcggcgcttgttgaagaactcttcgccctttcgcgccgacatcgctttccggattgccgacgtttgttcccagtcgaactgaccaatgtccttcacaattgacatgatctgttcttggtacaggaacacgccatacgtacccgacagatactgttcgacctgcggaatggtataggtcacaggctcgcgaccggccacgcgctcgatgtatttagtggccatgcccgaagacaacggacccggacgagcgagcgccgtgatgtggtcgatgttttcgaacgcggtaatgttgatcgcattggcgaccgagcggacggcctggccttcgaactggaagatgcctgacatcttgtcttcgttcagaacatccaaaaccgccttgtcgttcagcggcaagtcgtacaactcttgcgccgtcacgcaattcgcatcttgaattacgcccagcgttcgaagacctagcgcgtcaatcttgagaagattcaaatattccgaatcaggcttgtcgagctgcgcgacgccttcagaggtaaccgtacagaaatcgatcacttcatcgttgcagaccaggatgcctgccgcgtggacgccggagtgggatgggtgaatttcgaggtcgcccatgcaggcggacgcaatctcatacttttcgcggaagtcgcggccgggttgagtcttttcgaaagtgtcctccaatccttttccatatcgttcgtccgccgaagtatattcgatgatcgagtttttgatgttgtcggtgtcatggaatggaatgccgaagcgctttccgacgtgagcgataaccgacgcggcctttagtgtgttgatgttcccaagctttaccacgttccaagtgccgtatttctgctggagatattcgaacactagatagcgatgggtatcggcgaagtcgatatctatatcgggaagatcggaacgggaaatgtcgataaagcgctggaagagaaggcgatgcgggagcggatcgacctcggtaattcccagcaggtagcagaccaaagagccggccgaagagccgcgagccggaccgaccagcatatgcttcttggcgaaggcaaccagatcggccacaaccagaaagtagctgtcgaagtctttcagctgaatctgcttgatttcttcctggaaccgatcttcgtaaacttgggtccattccttgatgtggccgcgactgagacggtaggcttggccctcgcgagccagggcgacgatatcaccatccaggtggatcatcggcgctttcgccagctttacatcgaccagctgctcgaccaccgcatgcgtgttggcaacggctttgtcgaactcttcgcgggtcatgatatggcgaagacgggcccacaactcttcctcagtcgcgatgtggcgaaggccgaccgattcccgaaccttccaagccgaagcaaaatctgcatggtcgatggacggcatgtcgttgtaggaggtaatcaccacaggcttgccgaacgccctggcagtctccatggcgccgtgcgcggcgaccatcgacgcaggattgatgtcaatgtagtcgattccggccaagtccaagtgggcataagcctcgccggcaaacttgatgatgccgtcagcatcctggaattctttgggcgacaatccttgattctggacagtcttcgaagtaaggcgatagaattttctggtgtccttggctagcgcccaggccttcagcttcagctctttttctccatcatcggcgcatttgatcgggatttccatgccgaatccgcgaggcaattctgctttggtagctgcctgctcccagcggacgtggccccaggtcccatcatcgacgatggcgacaaagggcgattcgatttctttggcgcgctcgatgatttccggaaacctgccatatgcggcgccgtatgagtagccggagcgaacgcggagttgagggaaagacattatgcggcctccattgcttgatatgctcgatatactcccatgcgcttgcaaacttcgtggagcagccgcacgtcgtccaatgcccggtgcttctgaacataagggccgcagtagtgctcatacagatgctgcagccgcatgcggtggccgaacaatggcgccgactcttctacagtacagatatcgagcgatgggaagttgacttcttccaggccgagcttcccgcgagccaaatcgcaggtaagcatgaatttatcgaatggaaggttgtgggcgatatttgcgtcggccttcgaaaagaaatcgcgaactttctggcgttgatcgaggaacgatgggtgtttgattaagtcttcattcttcagacctgtgatctttgtaatgatttcttcgatcacaatcccagggttgcagataaactcgacttcatccaaaatcgtctcgccatcggtgatcactccggcgaattcaatgatcctcggttgctttctcagacttaccctctggtggaacgggagtcctgtggtctcagtatcccatacggcgaatctcatgtcggttccctcttatgtcgaaaggccggctgctttcgcgaccggcctgaagagtatactgcaacggcgcagggtttatgccttctgtccgtctttcggcgtgatgcggccggagtgcatggtggcgtggacgaacgctgaatagttgatcaagtcaaataccgaatcgtcatcctcaaacccactattcgccaggcgagtgagtttgccaactgtatgcatcacgaacagggcgagccgatggtcatctgcggtcttcgccaccatgccattcgggaagaggatttccatgatcttgccatacatcagatcatttcgaccataagcgctctggcggtcgcggaaaatttctgcggctgaggacaggttgttgagaacatcctccacgaaatcatcgggatcggcatcaccgtcaccaggccaggcggattccgtggcgaacggcgctgctgcgtcttcggtcggctcttcggctggctcttcggccggtgcttcgtagagcggagacggggcctgcgcaagcgcctcgtcgagggtaggggcggagtaggaggctgcggcgatcgtctcgcctgccatgtcgttgggagcgctaccggggtcgctaccggggtcgctatcggccgcgacggcgaagaacggcgcgttacatccttccaggtccagaatgtaggcgtcgatgcccaggcccttgtaggcgtcgatgatatcctggcgatcatcgaatgccgcgacgatcttggtcaggccgtcgatcttcttcagaatatctagcgcgactgagcgcttgaactccggcgccggctgggtgtttccatactcccgcatgatgagctcatattcgcgatgttcggcgatacccaggtcgcgatggattttcgccctggtctggaaatagttgttgtcggttcggccggtgatgaagaaaatcatcaggtcggcgtcgatggcgttacggattcgtgcgactgcatgcggattgagcttgtccttgtcgaggcgggaatggtactcgtcccattgcttttccagggcgaagcttttacggtggctatcgtcgaagacgcatccgtccagatcgaagatcatgatgccattcttgggttttcgtgccatattcagatttcctcgctttctgctttctgggtgatggttttctcgatgaaagcgccatcggaagttagacggaacagttcaccgttctggaccagatcgaacgatttcacgttcatggtgacgcgaattgattcgcggccgttggtcacttcgacttgtgcgacgtcaccaaccttctcgatgatgatcttcatgctacattgacttcccattgaccgctacaggattggcctcttgtttctccgatccccagaactcgcggcgcagctcttcctgctcggccgagcgatccatccatgggcggtagaacttgcactgatagatcggcgccaccggcaactcaatcacaggaatctggccgggcttcgtccccagagcggacatgaaggtgttatatgaaccctcctggatttcctcttcattcagaaccttcgatccatagcgcggaaaggcacaggagccagtagcgacgcagtgcggctggagcagactatcgaacatcggatagacttccagaaccagtcggcgcatttcgcggaaggcttcctgatactccccttgcgtacgaacacacaggcgaactttcgccatgtcgctcagagtgcgcagattgaacttggccgcgatcttcgtttccatgttggaagggatgatggcacgagcatcctggagcgatgcgccggcctccaagagcttctggtaactggtctgcgcgtcggcgatggcatcgtgccacaggcggttcaactcttcgcgggcgtgataggtcgggtccggctcgccattggccgtagcctgctcatcgaaatcccagcggaatgcttccggctgaacaacggcgctaacctccagggcgcgactggtttcctgctggtaagccccggtccgcgtccgaacgagttgatgggtgaaattcttgctcacaccctcgatctggaaaatgaagtccacgaattcgaacggcgagcgaatggtgtccagcatgtacttccagtggtcgagcttttcggcttcggtcatggtcgccgggtcttggccgcgcatgcgggttgatttagtgcccaggagaagttcccaggcgttctgggtgtaactgatcagagaaattttcatcagaaatcttccggaattggcgtgaaagtgaatttctccaccagtgccatggccaacgcttgtgcgtcttccttatgtagaccgtatctgtcgatttcttcggccagcaatctgcaagcctccagacggtcttcatactcttctgcttcgcacattgacgaaaagatagtgccgtcagaggtccggtacaccagctcaatggacatcacttcaatcctcagtagcagcggatgatttcggcgcgaatatcgcgacggtccaggtaatgctcaataatcttgtcgcgggcgcgctcggcctcttcgcggctaccgaacgacaggttgaatgaagtgaaaggcttatcatccaggcgtccatcgccaatcaggtacaggatgcctaccagcacgaaagacggcgaggtcttctgctgcgggtcgatttgcatttcgaggaaagacataggaacctcttcaggttggtctggtgcgtacattaatagcgctcctgctgagcggccactgtttccggttcgtagatgatcatatcaacgatttccgggcactggcttttcacccagtcgagggccgcaaccagcgttgtggcggcgccgaaggtgaaagtgcgataagactcatggatgtacggcgaacccatggagtcgcgctcggtcgtacgagaaatgactactttgatgttaacgatccggcccatcttcggcctccactttagcgatgatatcggacaggctcagcttctcgccattcaggaaataactggcacgaagcttcctgtcgccttctggcccgacgatccgggccgttattgtgagactcccgccgccaagggcctgcgtgtcacggaagaaagccagcagcgccctcttgagcgctgcctcgcgaggatcgactgccatcaacctaccacattccagccgtgctgggcacaccaaaccgcgccagttcccgcaggaagactgttaagaaggaacacaggggtcaggcggccatcctcggtcatatggatgaagtaacgtgcactttcaccgagccattcggccttggcgatggcgcgttcgaggttgaccttggtggcgtaggtcttggtgatgtttttgtcggtggagaaggttacttcgcgggccattttgtcgattccttttggttgaagggtttcgcgtttcgatgagggaatactactctcacctggctcagaagtaaagcactttgtgtaaattatttcacgaacatcttcttggccttctgataagacgaagaagtcatcaggcgctcgatgacgtccatgtccgaaaccagatcgtccagaaggacgtttcgccaggtcgcgaaccgaccgagcgagaagatgccggcttcatgggtgagattccagatcatggattcgcgctcgtcgcgaccgagcggaatgattttacccttggtctggacggtcggctcgccgtccggaatgagattcttcttcctgatgccgaaggccgagcaaacttcatccaggtcccagttgctgtcccattcgatggtttcgatttcaccatctacagtctccacgacgcccttagtaacggattcgacgataagggtgtcgccggtgatggacgctcgaaacgttcccacttcgggaccagggaaatacaccgtctggaagacatcgcaaggaatggaaagcttgtatcgactcacgacgatggaggttccttcgccgaatgacgggtcgatccccaggtccagtcctgccgcagccagattggcgcggaatggtgctgtgctgatgatattaacgtgatcatcttgccggcgaaggaactggaagaaagaggcgtcgaaaggcctgccccagctgatgcgattcgctagcttagcgaccagctgctcatagtagtcggccggcgcgatccagcgcttttcggtcgccatgttccagatggaccgatccgacaggccgcccgttactttcctggagtacatgttgcagtagtcgatgcgcggctgggaaacgaactcgccgtcaatgtagatggccttgtgtacggtgacttcgcggaatgggatgccggtgagttggccgatgactggtgagcggaaccgcaagagcgcgttgtggcgttccttgctcgtcggcgtcgccgcgtcgatgatttgggcttgagggaaacgatgcgcggcgatcaggccggcgagtccggctcccacgatgataactttgtgatcaggaatcatgagatgttccttatgagtgtacagaacttgggaggataaaaaagggacccattttcatgagtcccttgaagagctagacgattcggtctcagaagagcggcggcttactcttcttcaccatcggaaccgtcggcgccctgaccttcaccgtcgtgctcctggccttcatcggccttctcgtcatcgccctggccagcttcgtcttccttcgaagcgatggcaaccagatcgacccagcccatgatttccagcttgctcagatagctgcgaaccgaggtgccgtacagcaggtgggccaccttctcgccgaaggattcgatttcgaccggctcaccaacggtgcagtgctcgttgatgtaagcaaacaccttgccgcgagtcgagaaggcctgcggggttccatggccgtcgccggtcgggatgaagtgggtggcgcgcggacggcgcgaaccattggacttcaggtcttcgcggcgggcttcggccttcgcgcggcgctcttcctgctcttccttgcgacgcttcttctcggcctcgcgctctgccttcttctcttcggccaggcgcttgcgctcttcttcgcgtgcggctttctgggcttcctgcgcggctttcttctcttcggccttcctggcgcgctcggcttccttctcggccttcttctgctcgcgctcggcttccttcgccttcgccttctcggcctgctcggcttccttggccttcgccttctcagcgcgctcggcttccttggcggcggccttttccttcgccttctcggcgcgctcggcttccttcttctcgcgctcggccttgcgcttctcttcgcgctcggctttcttctgctcggcttccttggccttggcttcggccttctcggcgcgctcgcgctctttacgttgacgctcagcggccttctcggccttgcgcagggcggctgcttgttccttggtcagctcttcgccttgggtctgttcgttctggtctttctgttccatgttcttactccgggaatgttttaaagggatggcttattggcctgtgaggggattatctctaaactaactgaagaagggaatacccttagcctgaactttcctaaatattttctttcgggaaagtccaaactctagggaacttatttatgttcgagaagttcctagcttttacgcaagaacagtaagtattcgattgcgcgagttatcccagtatacatcaactgactataagggatggacggcaagttttcttctaacatggcgacccgtttccattctgatccctgcgacttgtggaacgtcatcgcccatccgaagtcgaatccgccaatggccttctgcgcctccagccgcacgtcttcctcgaccgaaaaactcagaggattgaacttaacccagcgttcaaagttcgtaccgataatgcgaactttggcgaacaacatttcatcaggctcgtcatcatcttcttgcccttcaggaaccggcttgaagtccagcagaatggcttgttcgccgttcatgattccatattcgtgctggttcccagtacacaccagcttctcgccgattcccggctgcgcacctttgtagccgaggattcggcgcgccctggcgttcaaacggcggcgagtattgttgtaggcgcaaaggatcacgccgtcatcgtccaagaacgtccgcatttcgtcgtccgacatgtcgaatccggcccggaccagaatgtcgtcatactcgcggcagggtaggcgcttgccctggcggacgaacatagacgcccgaacgatattgccggcgttgcgctcgatttcggtcatgatggtgtcacagctgttctcatggaaaatctggacgccgcgcacaggaggaacttggccaaagtcgccaatctccagaaccggaattcggtgcgacagcaagcgctcctcatcccactcgccgatcatggacgactcgtcgagcacgaccaacttcggcttctcgtcgagcgagtctttgttggcaaacatgatttcgccgtcttcatcttcgccaatcggccgatagatgaagctgtggagggtccgggcattggtgcaacccttctcgcgaagccgcgctgctgctttcccggttggcgcgacgaagaccgtccagtccatcgagcagcaaagttcggcgatgatcttcgcgatggaagtcttaccagttccggcgaaaccagcgagtcgatagacctggcggcggtgcgctcgatcacaccaaccgcgataccagtccacaacggaattgatcgcgtcgatctgctgactgtttggtcgaaagccgaatcgctcttcgatctgatcgacggtgaagttagatgctgacatatttgcgttctccaacgctaggtttaattgaatcgaggctcagtttaagcgcgccgtccacagaccagccagtatcacgacgatacttgcagccttgcggatcgacatagaagttcttcgtgcgccgcagcagaacataatgccaagcagctccaagcgcgtggacccttcctttatacgggaaagccttcgcctgctctgcggcctctttggccccagggagccagcggacggtcgaaaggaccaggaccgcacccttgacagcctgggaagcggcgcggccgtcctgtgggctatagcgatgtctatcggggtctacccagtggtggttgccgcgccggagcttcaccgtccgggaccggccctcaaacaccacagtaccttcgtgagtaaaaatatgttccgccatggaatgttccttataacgtacagttctgctttacctctgcgcaagaagagtatactatcagctgactcgtcaaagcgagctaatttaatccgactttacttcggcaggaaagtggccgatactagcgccgccgcctgtactgccctccaaaacagaggatgcattaaatgcaagaatgcaagatttcccgcgaccaactcccggtcggcaatccgaatcccaatgtcgataagactcgcgacccgaacctaaagcccggctacctgcgtcgcagtcgcgagctggacccggctcttgccgttcgcattcgtcgcgagctgatccacgctgaagcctccgacttggccatggccgggtgggtcaattcccagtccagcctctatggatcgaaagcgttcccgcgccattccgtcgttcgcgtgactgggatggcggaatctgaaacgaacgtcggaatgctcatcggattcatcgagcatcgcgagcatggcgaatgggcggtcatggaagccggaacgaaagaaggcggcgccgtcatcattccagtcgatcacatcatgcgagcgtcattcgccgaagccgaagagttctctgaaaagtgggagcggaaccttggatggcgtctcctgcgccagctccgcgagtgcggcgccctggccgggactgaagacgagttcctgcggcggatcattaatcgatacgttcgtgatcgcacgatcctcgatcaccacaaagtcggcgcggacaaaacctacactgatgcagttctcaaaagcatcggtgaaacatggccgaaaattccttcggggaaattcgtcggacaccgagtcgcgcagctcctgatcggccacaagctaggtcgagcggggaccatcctgaatgacctggtggacttcctggagaagttcgcggccgggcgcgataaagttctcaacatcgccatctgtaattgaggtgattggcatgaacaataaaccgacgctgaatcaccaccatcaaactgcattgttgtatctgtacaacaatcccaaccaaccgccattcaccggtcccaataataaggcgctaaatgaacttcgacagatggggtatgtaaaggcgcaagagttcgaaaactgggcaggaaccggccgtatcagggctgaatggatgctcaccaaagccgggattgagcgcgtcgaagtcgggcttctggggaagtgcgacgcttgcaagggaatcggtcagactttgcttcgggggaagtgtactgtttgcaatggccgcggccaggggtggatcagcgaatggtcgcagaagccaatcgaagataatcctcaaattgtccctaagttcgaaaagaccgatgcgaataggcttgcagacaccattgaagaaatcggccggctggaaaaggcactggccgaatccgaaaaacgcgggagcgagctggccgcgagctattgtgacggcgtagtcggcgatgaatacggccatccttattgtcgttataaagtggaacgcgacgccgccttggccgaagtcgagcgcctgcgagaatcaaaaggcgatccctctggcagcttcgacaggtgtatgaagatgatgtacgagcgcgacgagaatgcaaaacgactcgacgccgccttggccagggtcgcggagctggagaaagaactggcgatggcacgcgacgcagcagcaaagggtgatgctgcccgccacgctgctggcggcatggaaatggagattcatgagctgaagacgaagctggccgagctggagaaacaagagccggtggcatggggagccttccatttcggcgggaagcgcgacggcaagctgtatgcgcaatgcgaaactgaggctcagatagaggcgtatatcctcgacatgcaccgaagcagcgactcattgacgctcaggaaaggccccctctacaccgcgcctgtagtccaggctcaacccagcgtgccggaggggtggaagctcgtaccgatggacccgacatcgcagatgacctctgtcggtcagtccctgcgttatgactcggtaaacagcatcggcgagatttaccggcaaatgctcgcagtagcgccctcacccatcgatccggccgcgcatcctcagccgtgccagcaaccacaggcccatcctgcccgctgcgggtgcgagcggtaagtgccaagtaaggagtttatgtaatggaacagcagaaaccttcggaagtagatggagccatcgccatgaccgatctcgatattcgcattcttcgaaaggcgaagccagaagcgcaggacgagtgcgccgtgtccatgttcgcaacggcgatccgccagaaactgcagcgctcccgcgataaaggccgaggcggctggatcgactgcgacgaagatattctgatcaacggattcgccgaacatgcgctgaagggaaatgagaacaacctcctggacctggcgacgttcctgatgttcatgtgggtgcgcggcatcgatgatgcgaagattcccccggcgctcgaaaaggcgagacagcacaagatcatggaagcctggagtcgaatccatgaagacggcctgaactccgccagaaaggcgagtgctgcgcgacagttcatggaagtgccacgacgcaaggggcgcccggagcgacttgcatgaagcctcacgaaataagactggcccaggccgaagagttcctgcgcgaactcggccgagggattccggacgacgaacgggtaatggtcggctacgctgaagaggccacagtccagaccgacgagaacggccgcaagctcaacgccggctggtggcctgtgccctggaaggaagggaaatacatcaactccagatccaacgcctacgcgtgcatctcgtcgtccatcaagacgcccaacccgaagaccggccagatgcgatactggcgcggcgaagcctctttcggccacggcctggcgttgatggtcgatgacatcggctcaggcaaagggtccaagggtgacttcgaccgcgacgagttccgcgagcgactggagccgaccgcgattgtggagacttcgccgaacaactaccagttctggtatttcttcaaagagccgatgtcccacatgctccagttcaaggcgctgctctattcgttcgtggaccaggtgctaaagaaaggcggcgacaacaccgtcaaggacgtcagccgttacggccggatgcccttcggcttcaacaacaagcgcgggaaagacggcaagttcaaatatgccgacgaaaacggcaagcccgaactcgtgcgactgttcagcgccgactattccaagcgctactcgccagaagagatcgcccaggcattcggcgtccgcatcatcatgccacagatgaagaaggtggagataaaccgcgacgattgggtgtatgaccaagtatggctgaagtatgccgagcacatctgcacgaaatacaagatgggcgaagcggcaggcggccaggtccagcagaatatgtccggcaaataccgcatccgctgtccctggggcgacgagcacaccaacggcgatcctttcggcgcatactttcgcggaccgatacctggcgccgagcacgaatatgtgttcggttgcgggcacgatacttgccgcaaagagcatcgacggacgtgggcggccttcactgatgaagtcgtcctgccgtatattgtcgaacaacttgaacgcatcaaccgccgtcatatcggcgaggagtagtaactatgcaaaatgatcctggaattctgatcaccgccatcggctcattgcttctcggccttctcgtcttcttcgaaggcctgaatggctggaaaataccagtagcgaacttcctcgcgtcgcttctgtgcttcttcgtcggtctttctgctttaacgtgctggttcgtcttggcgtttgacgtgttttagtcgacgaacggtccggaaattttcggatggggacggaacttattagctctgccggtttaggtaggagataatagccgtccctttcgcctcaatatgtagaggcagtgttatatccgatcatgtaaagcagaaggcggcaaacctaacatgattatcgacgacgataacattcttgatgatgacgaatcggggtccggtgagttcgatctcacgcaaatagaagatgctggaatggaccctttgatgaccgccgcaagtaaagcggccgatgatgcgattgcgaggaatgagacgtaccgagcacaaaaggcggcaaagtatgccgaggcgtatgcggaaccagatctgaagaagcgagcgcgattgttgatgctcgaccaggcgttcgatcttccggtcagccgggtggtgaaagggccgttcgatgacttcatcacgaaatacagctcgacgtcagacagcaactacctcgcggtctatgatacgttgttctgtaagggcgacggaaccgtcccacacccgcacttcgacgagtttcgcggccggctggtggaccatcgcggcgtggcattcaacaacaagaccctcgatccgattgacctgatgggcgccctcgcggctgcggccttggacgatccctcgattaagaagacgattgagacttgctgcgtttgggcgcgtcgatatcgccgcaactcgctgatcgagacgtttgagaagaagataccggattgggacggcgaagagcggatcgaaacgctgctgatcgacttgttcaaaccgttcgacaccgagcttaaccggatggtgagcaagtatttctggctgagcttgtactgccgcatcaactaccctggaatctcggcgccgatctcgttggcgttgattggtgggcaggatgcggggaaatcatatttcggcttgctgatctgtaaggaactgtccggtgggcgcgatctggctccagttcagctcgacctgagccgacacgaccagacgccgttcctgcgcaacatcaccggcaactcggttattgcgaacgtcggcgaaatgtccggcttcaaaaagggcgacatggaacgcatcaaagaattcctggtgcgatcttcggatacattcgatcagaagtttgagcctggcgaaacgatcaagcgacaatggatcaccatcatggacggcaacggatacgatggactccagcgggacgactccggcaaccgtcgattctatccgatgttcgtggcacaacttcccgacgaagatggaaagccgaactgggttaagccgggcgatggtaatgaaccgttcaaggtggacttcaccgacttcggccgcaaattctggcaagcgatggctgaatgccgcgcatggatcgaagagcatggtgtcgatggatacttgaatatggtgtctgaagcgaaccgcgaagtgcagaacttctctatttcggaaatggagaatgcgcgtggcgtagttcgtgacgatacgattgacatgtatctgatcaatgttctgatcagttgcgagttcgaagagatcaagcctggcaggaatgccaagaatcctggatggagggctgacaccgtcgccattctgaaatggttcgatattctcgccaggaagaagccgatttctcgtcaccttactccgcacctgaaggcgctgggattcatcccgaataagaacggcctggctggttggtgcttacctgtcgataaggttgcgcctggatggacgaatggcatgcacacgacgttgccgccgttcaatgatgcgcttgtgtacttgctgagaaagggcgacccggatatcactgatgaggctgccatggcaaaaattcgggcagtccgggccgagcgagccaagattttgggcgaggatttctgatgtagtggtggagtgtacttggactaggccgccttcgggcggtcttttctttggtgcggcgtacattcgattccggtggtggccgagtgagattcgaaagctatccggtaattagggtggggtggcgacggtgtattgtggaaagttcgtggacgttggatcgaggattttgtgtggtgagtggatgaattgtggaaaatcgacgattctgactggatttttggggtcggccgcgtcgagcttaccggtaatttacgaaaccggtaattagggcctagccttagtgccgcgtgggtttgaagggctttaacggataatttccggtttcggatgcagccgcaaaagtatactacatcgaaaat

SEQ ID NO: 7 - DNA, 45300 bp, synthetic construct
  source 1..45300: mol_type = other DNA
ccccagagtcccccaggctaacgttggggtggccctctcccaggggtactagcccctagggctcccccaggataccataaggctagtccgggggtcaaccccttgagataccgttcatcggatttacccaagggtattcataagaaaaatcttagggtattcagggggtggatcggggggaatatcccttgatcccccattgacatcccacgataccctatgttaatcaaagactgtctatatccggggggtttgctgggggactgatagtccatagactgtatgaaaaaccagtagacaaccccgtaaagaatatggcgtaatggcgccgccttgagggaaaccgataggcgccgggggaaaccccaggggcttgacaaaccccgcggaaccgggtctaatggcccccacgttctacctaggccaccatcccccttgccgcagtgctatccgggggctgccaaccagggaagagcgcccaagtcacccaagtataagaccggcaggcatcgggggttgacaagggatcggtgaagcggtatagttcgccccacgtcaacggcataacgtgcatgactccctcgggattagcgtagccttgacaagcaagactgatggccgctcagcaaaagagcctatgccggagaaccggacgaactagcccccgtcagagggttgacaagccaagcccacggctctaacatgggcacccatcaggtgacggccacctggggaaagtgtaccttcgacggtccttggactgaacaagggagaaacccgaaagctgagggaataacgcacactccgaaagagcgtaggtcctggccttgtaccatagaaggcccgactggtagcctgtcccagcgcaatcaattgacagaccccgtggttcaagcgcgacggggagtatgatgggttagtgtcgaaggcttagcgcagagggcgaagcttggtagatacggcgaaggtaggggcgagaggattcgaagacccccgaggataaccgaggacagaccataacgacgacactaacgcggaaaggggccggctacgccaatgccaaggtttgccgataaacccctttgtcgtatcgggatgtgtgtcccggctgatgattcctaaaggatgaaacgaccatgactcaggctcttgacaaaaagctgcgtcgcaaggccaaccgcaaggccaaggctttgggttacaacctggggaatctggggaaggcccaacagcgtagtgagcaaaagtttgggatcattgcaagttgtaacaagatcctcgacgacaagaccacttcgttgcaagaaaaggcaggtgcgcgtaaacgcaaggctcttatgagtaccgactggcgtaaccgcgaggttactaaccttcggaactggtataagccgagcaaatgcggtaactctgccgtcatcactgtcgaagtaaacaactgaggtaattacaatgagcgactacaagcgtatcaatgggatcatcaagaccattgccaaccggggtgctgccctggataagctggttcaaaccaccggcatggatatcctcaagcatatcgacgagcatggcgaggtatccttggcatgcaagctgttcaacgcaatgccgcaaggctcccgccggctggccctggcccactggttcgtagacaacggcaagatcgcggccaataccgacaaggaaaaggccaaggaattcccgttcgtcttcgccaaggataaggccactcgactggagcgtgcagccgagaaaccctggttcaagtacaagaaagagcgtgacgtggccgacgagttctccctggaccaagccatcgccgccttcaaggccaagatccagcgtgccatcgacaagggccagctccaggcagcggacgagcgtatcgccgtgatccaacgtctggaagtgaaggacgaagcgaaggcagcgtgacggcgggtgagcctggggtgtgtactatgacgcgccccaggtctatccctagagtgcatggtaggtccgtgcattgtagcgatagaccatcaggaggtttccgtgggttactgtatctttgggcaggttcattccatccagggccaggactatgtttctggtttttgcgatgcaagggtaggtttcgacgctggtattgtgtaccgctggacattcaaagacggtaaatttgcctgcggtgtttacactctggaaggtatccgtctgaacatctgcgggtggctcaaatgaagtcaccctatgaagcggcccatgaacgtgctcagatgattaaccgtctcaagaaactcactaggatgatccgggtgcatcccgatcccaggtggattgttgagcgtcaggaactcataaggaaactaagcaaatgaccgtcgctatcattgtatccagcattggtatcgcatacttcttcttccgtgattggaaggaggaaatgggtatctaatcccaactgatgaggccaaggtgattcctggccgaaacccccaccggacctatggtcgcaggctggggcgtcttgggaaatcaactaaggaaaccatcccgtgaaacgcaatgactaccgcaaggtgaatcggaacatgcaagccatcgaggccatcgaccgcaagattgccaaggctgtccgtgagttgactaactccggaggaaaccacgttggtaagaccctggagctgaatcggctgcgggctaagcgtgcatcgctggctaaggtaagggcacggtgagtacatggaagtctattgtatgtgccgccatacgctcacgttccaatgggttgctgctggtaacgtgggtggttctcttcgtcctgtcctcgatcttggacagcatcccttttgcatcgtgaggtagtcccatggatatcaaggtgtggccccgcaacggagtaaccctcagttccctggttaagtcccaaaccttcctcatgaatgagggtgtgtacatggtctgcgaactccgcagcaccaggatgaagggtgaatctgatgaggttgtcgttgttaaccttaagactggcaacgtcatttacatgccaccggtgagtctggtatacccagtaactgccgagctgaattgctacgaggtgtagccatgaacggtttctcattcgcagccatcatcctgtcaggactggctatcggttgggtgattggtgcgaccacggtggttcgcaaagatgcacccgtggatgtaagcaggggcgaagtggttggcaagatggacggggtgagtatctacagactctacgacccagaggaagggactgtttgttatggcacctaccggggccttagctgcctgcgtcctcaaggtggtgtaaggtgatcgtgactaagggttgtagtaacgggtcagtgggggtttttaaaaagaaagtagtgattggtaggcatatggtttttgtttaccagaatgtttgctacatctaccaagggggtactccctggtgcaaaaccccgaccctatcttcggcgaaggccatgattaataattatgaggtggccgtgaaggactcgtccaacagggtcaaagatatgttcggcgacctcatggtcatcccaatcgaggaggtgtaacatgaaattcaactacaacatgctcaccagcaagatccgggttgcctcttgggatgatctggagtctggtcgtctgtacatccgccaggacgaggacgggactgcaaacccgcgcgtctttatgtacatggaagttgcagatgaattcttcatcgtggatatcttcggtggacccgtgggtgccgtatacttcagcggagatgttgactgcgggttctatcccgtgaccatcaaggctgcgtccatcgaagtccatcggttcggccctccggcctagggtgtgtactatgacgcccgcccaatacatctaccactggttggtatccaaggttgtcatccgtcgataccagcctaaccccaagctgtaccctaactggtctgttactcatatccgtgtgtcgatcttcggcaaacgtgcaggtatcgtctatgaaatccactgaaccccaagtttactggctggctagcgccgatggtgaattccacctcaaggtcatcaacttcggccagttcatcaccgaaaccctttacgaactcggcattccggtggaccacaaggttcatcgggtccactgaggtgtctatgacaggtactatcttcaccgtagcggtgtcccaacctcgaagtgatctgcacatcagcaaacgccaggatggcgcgtatgtaagcatcaagaacgatgaggtatacctcaaacaaggcgggaccgtgacggttattaccaaggttggtttctccatcttcgcagccaatatggtatgcaaccatggggaaatctacccggtgaaggaggtccatatctctgccaggatgtggggcccgtatgaagagtaaaccaatgatcggccaaatcatggcccaggagcgccgcatgaagcgtcgggtagagaaacgcgggttcaacatgacccgtcaggaaggcccccaggagccccgtggtgagctcggattcaccttggcagccgtaggtgtggagtcgagccgatcggcttacctgcggcacgccagagaggctatgatccaatccggggaggattgcccccactgtatgccagtcttcggtcctaaagagggccgttgctgcaactgcgcgagggattggtgatgacaactccagaaatcctcatggtgtccgccgttaccctctccgggttggcggcctacgggttcacatcctgcctgatctattcgatattggaagagtgtgggatgaggtgggggtcagatcgtgattgggcagtcctactgtccttcttctggcccttgtccctgcctggggtgatcctgtacttcacctgctacatacccctcaaggaagtcttcatatcagccaagcgactcttcggcggccactacaggaggctctaatggaaagtgtaatcgtctatgcggcggcggccttcttcctcatgatgtgctgcttcatctacttcctcgtggcagccgtaggagtgtgcatgaccttggctctcttcgaggatagtggaaggatcaaggaagagcacgccattgtcattggtgcattgtggcccttgtcactcccattgatctccttcggcttgatggtctactacctcctcttcccgatgaaactcttcatcatctccctataccaagttatcaaaggaatcatcaaatgagagagcacgacttcccctcttgctgcaccgccaaaatctacaccggtatgggcccgtcggggaccgctgactaccaatcgatcagggactccgaggggttcagccccaagaagtttgcgatggatgttatccaggccctggccgatcagtaccacgaaggtgaggccctcatggtcttctccctgaacaacgagcagcgggtcgccgaccgtatcctcagtcgcctggggagcaccagcaacccttgggccagcagcgacaaccacgacaccctcgtgcgagtccacatcatccgggtccgtgacacctggggcaagctgtgccgggagaacctggtccgctacgactgggaccacggatccatcccgagcgaggacttcatcagcaagttggttaaaggtttgtaacatttgacctattgactttctgctggatgtgtggtataataaccttaaggtgcctggggttgctcttatacctataggataccttaaggtctacctttatatcttacttattatgtgaggtatcaaatgctcaagctctatacccggactatgctggtggctatccccctgtggattctctaccttctgatcctcccctacctgatgacctcggattcaacctcggctagcgccctgggtgctggtctgatggtggcctccatcttctatggtctcttcttcttgaagtacctgtacaactacagcaagcggctggagaaaagggtaaagcccctgggggtagatgatgagtgagtcggagaaacccatcgtcagcttcgagaagttcatgcaggtcatgtatgaccgcgacgagtacgctaagcgtctggaggtagccacggccaagatccaggaattggagaagtcgttggctatggcactcgacgcagcagcgaagggtgatgatgctcgccatcagtgcggaggaatggaaatggagatccaggagcttcgtgatgaattggcaggactgaaaaatgaagtaaaggttcgtacaacccaagaagtagtcccccacttctggcgtcaccgagggggtggtcgaatcttcgtgtcaggcaggctgaaccttgacaccgagacacttcccttcatctgcctggagactggcaacagttgggacattcaacctgagcgggtggaatatatcttcgagttacttcccaagggtcatatctttacggtggaggtctgatgtactatgacgaacagcaatggggaccgcacccaagggctccgccggtgttaccgcactagcgaattcaccggtgatatatacgacgcgatccacaatatcccagcaatcgctggatcccccaagaggccaccggatgatgattgggtatactacctcctgatggtagtcctcttcatcagcttgtgcatggccttccttactcccctcatcaccagcaaggatacagcctatgaaagccaaagtcttctacgagcgactgaaaacctccggcgagctgaccctgaatggcggcatctacaaggccaccaaggttcctgcgccccacaagggtaagcagcagcgtcgcggccccttctaagagggcgttagtaatatccctaggaactccctggggatattgctggctaactcaaccaaggagaaacaagatggctcgactcaagtacgcaatgaaccacgggaactccctcgtcatgatgaccagtagctcctgctttggcgctctggaggctaagtacttctcgaactggaacggttggttctacatccaagctcgccgtacccccaagtacattcgccagatgaacaagatgttctctgacattaccctgacggtggccaacaccaaggtaaaagtccccaagatccaccactggcgcatggggtccaaggccaagagcgcaccctggaatcgattccccaaccagaaggtattcaccgatgaccgcatgtggggtaacgattacgtgtgcggtatctacatgaacccccggaccactaccccggaggtctggtatcccgtggtcaaactcctcttcaagtgggtatcctctggtgcgttcgattgggaggaagaggcccatctggagttggccgataagcacggattcctcctgtcctacgtggcaatgtcctgctacaacctgatggccaatgggtacaccgagcaccccctgcgaaacgtccacggcaaggtgtggaagcccgaggccgtcgaaagactcctcaagggcgactgggaaatgcccaaggtcgaagaccaaggaacctatggccggtggttgaagcaggaggatggcacctgggacttcatcctgggggttactcctacttactatggtttcaataaggagaagatcgagggtatgcggaagacgatggacaaggagttccagatcaccatctaccctcagccctgctacctggggaaatgggaggatagccactggcccttcttccagagcaacaagctgcttcctcagcacgtaccctccgtgaagaacgcgggggagcgtatcgagttgataccccctcacctgtctcgtccgatgaatggggcacgaaaagagtccttcctccccagtctggatgcaatctgtcaacacctcaagaaactcatgggagattaatcatgggaatgtatgcggcagaagtaatcgaccgggagggaagacccttctcccaagttaagaacctcaatggtcatacccgttgctacggggacttcgaggacaggaatggtgaagcgggttgtgttgtccacttcatttcccacactgccaacgaggtcacccgaaggatcgctgtggacttcctcaacagtgatttcgtcaaccggatgaccgatggtgctatcctggcggcgtccgaggatctccccaaggagtggtgggatggcaatggtgatctcaagcccatcggggacttcgaactccctcgggcaatgagtcgcttctacgtccgggtagatatggacaagcctggcggtcccatcatcaacgccctgcggatggcccacatgctggtccacgtcagccaccagccgaacctacttcagaagttggaccccgtggttgcagtcctcgtggcctgcgcaaggattggccacaacttctattgcgacagctacgtcagcatgatccccgtgactgctggccagtatcgtgagcaatgcgagaaggagggcatagaggtagatgactcctacctgacggagacgatggccaaggctttgggcctggggcggattgatagctggggtgattatgtacaagccccagagcttctccacgggcccaccctgtacgatatccaagatatcatgggtcgtactcgggaaaccttcaagcgtctctacagcggccacaaggaccctctctggcaggggtataccaaagactgccgagctatcgaggaactcggcctggatgggtacgaggatgatgatatcatcagcgtgtccgcaggtatcacttacgacctcaacgacaagaaccgtgagcaatatgaggtaatgcctctcgatagttacgaggacaccctcattcaattcttcaccgaaaaccaactgattaaataggagaactgccatgaaaaatatccctctgattggtgctgacccggaagtcttcgtaggcttcggtcgtgaacccgcatctgccatcggcttcgtcggtggtactaaagacgagcccatcccggtggaaggtggggcgttgcaggaggataacgttctgctggagtacaacattcatccggccagctcctttgagcaattccaccacggcatcaccagcgtcctgaacatcggacgcgacaccattcgccccttcggcctggaagtcatcgacaacctgtcctcccacctctacgaggaagatgttctccgtggcttcggtgatgcagcattcgtcttcggttgtgagccggactacaacgcctggactcgcagccgtaaccgtatgccgaccgatgcacccccgacccttcgtaccgctggcggtcacatccacatcggcttcgctgacgacctgcctgttaccaaggccaactgtcgtgcggtgatccaagcctgcgatatctacctgggcctggcctcggtgctgatcgacggagacaccgaccggaagctgctctacggtaaggccggtgcctgccgctacaagccctacggtgtcgagtaccgtaccttgtccaacttctggatcttctccgatgagttgattaagtgggcatacgactcggccctcaaggcttactccgtggtagtcgaaggcggcctggatgacgccatcgcagccgtaggtggtgcagacgaggtacaacgtatcatcaacgagaacgacgcagtggcagctctggcggccctggagaagctgggggtggctcatgaatgacctgaacaacgatcatcctttggccggcgacttcaccatgtactacggaaacacctacgtcttcttcgagaaggagggtgttccccatgtgatgtacgtcgagggaaccgaggaagatggcgacgatacccggttcgatgggttcgttcttctgggacgtacttacaccctggatgacaacgagggccggtattctcgggttcgcttcagtgatgtagtgaccgacagaccggccagcgggtattatgacattcacgggacgggcttccgtaatgtttacgtcaccttcgctgtcaacaaccgctctcagaagaagggtttggacccccggaacttcatcctgaacgggattggcagcaacggcagcctgtacccggtgaatatcctccggatcttccttcaatcccgagacatgatctccagcccatcccatcgggacttctacaccgaggaaggcaagcgagtcttctggaagggtatgttggtgggtgatctggtggaaggccaactggttgtcaaagatgaattcaagaaacaggagggactgctatgtcggctctatactctctgattggccaccgaaagcgtgaccttcccgagctgcaagcccacgaggaactcccagcaatcgcatccttcgggttcgaggttgagctggagggttccgacgagtggccacgtatagatgggtggaaccgcaaggaagatggttctctccgtagtggaatggaatacgtcttcgatggccccgcgagcggtgcagaggcaatccaacgggtagatcgtttcgctgctaccatggaagctgtcaaaccggcacccaccttccgttgctccactcaccttcacatggacatgcgggactcttcgatggatgttctcatcaagaccgtcctgacctacatggtgttcgaggatgtgttcttcgaccattgtgaaccctaccgccgggatagcaacttctgtatccctttcttctccaacgattggctggctcaagccttcggtcgtcgtctcctggccgtcaatgaagacggtcttcgggttcgagctctgatgaactggcccaagtactccgccctgaacctccagaccgctacaaccttcgggtccatcgagttccggggctcccatgcgatgacctctcgggatgaaatggtcgggctgatgcaacgtatgctctgcctcaaggccttcgccaaaggtcacgaaggggttgacacctctgagttcctggaggtgctggccaatgtggatctgcgggatgtgttccacctgggattgtctccggactacaacatgtctccgggtggccgtgagatgggtctggcggctgctgccctggctatcagcacgggtattcaggttgccaatggtgttgacccgatggaggaagagcaggagagacagcgccgactccgtgagcaagaacgtgctgcgcagaacctccgtcgtgcagaacttgttgcggcagccacaactcctctgagtttctccgaggaaggcgtcaatcggttcaacatcgcgagggctccgggtgaggtaagcaacctgggtaacgccttgatgaccgtatctgccctgcgtggtatgggttatcaggatgcaaccgtccgtaacgtcttcgatctcactgacgagagggtagcggctcaggttgtgaatgccttcgtcaacctgatgacggaccataccaactacgtcctccgtacctacggcgtaaacattcaccaagaactttactaaggagtaatacctatgtgcggactagtaggcttctgttctacaactaacgcgagtgataacgaaatcgctcttctcaagaacctcctggcggctgatatcgtccgtggtgcccatgccacgggcttggccaaggtcgatccgatcaagaacgaggtcagtattcacaaaatggctgtagatgcctacgacttcctggcccatgaggacaccaagaagttcctcacggaaaaccgtgcccgcatctacatcggccacaaccgttacgcaaccatgggtgacaaaggggatcatgagaatgcccaccccttccaagtggatcacatcacaatggttcacaatggcaccgtggatgcctggggtatcgatctgctggaaggtagtgatactctcaaggttgactcccacatggttgcagccaccatcgccaagtatgggattcgtagcaccctggaggaacgcttctcgggtgctgctgccctggtctggtgggatgcaaaagaacgcagcctgaacttcgttcgtaactcggaacgacccctattcttcgctgtgactaccacggggactatcgtatgggcatccgaggattggatgctcaagtctttcctggagcgtgcatccagcaagatcaagcttcgcggtaacatcgttgagctgtctgaagaaactctggtatccatcccgttcacggaggccggcgtgcggaagggtgtagagcccatcagctctccggtcacctttctcgacctaccgattcccgaaagttatcgtcaggcagaaaagttctggggcaactacgtcggagcggaagcctggaagcaatatctcggggactactatgacgatcaaggaagcaaaggcagccaagcgggaacgcaagctggccaaaaagcttcgtcgtcaagagcagcttcggatgaggcgtatgcaaggaataccctgcgaatcaacaacaatctccgagccgcaggaagctaccgtaagcatcacagcctcctcaccttccgaatcgtcgaagtccagccctacaaggccgaccctggctttggtacagtcgtcggaattgacctcgacgaaggaatggtcatcgaagcctacggactcaacgtcgaagctctcgaagggtacaccaccctccgtggaagtatctccaacgcctacttcatcggtgaagaccgcgattttaaggtggtggtcgaaggtgttgccgtaagctgccttgacccaaagcacaaggccagtccggtggatgggaccccgttgcagaccattggaacgcggatatccccttctaaagttcttcagatgaagtccgaggctggcaatactccaccggcgaatatctcgtatcccctgaagtgccaagggcataccttcgtgaacagctacgtcttccaagactttgtatgtcgtggttgcgctgtctgcggggatatccctacagcctatgaccaacggaaccgtgatcttacggtgtacgaaggtagcaagttcaccggcgagcttaacgagtgcgagttcgtctgtggcaaatgtgtccgtgaaggactttccgaagcagtataaggaggtcacatgacccagcaattcaaacgtcaagtagtaatccacatggaaaccgacgcaacccgcaagtatcccttctcccaggagactctggataagatggcggatatccgtcgcctgaaagaacaggagctgaacgaagccaatccgggtgaagaattcctggtgccggccccgatcgtcctggctgaagcggtggatatgctccacgagtactacttcgagtaagtgcgttagtaatgcccctaggtaactgggggtattgctggcttcactaacctcaacgaaacaggagatacatcatgatctatatctacaaaggcaaccgtgcatccaatggtgctgtagcccttcgtaatgccctgggtggacgaatccttcgttccgagggttcgacctaccgtggccgtcagggttccatggttatcaactgggggactcagaaccaagaggctgaacgtctccgtcgccttgcccccatcttcctgaacaatccccaggttgtatcgatggtcaccaagaaggatgaattcttccgccatatgcaaactgtaggcccggacctgatccctcgctggactaacaacttcgatgacgcagaagccctggttaacagtggtggtggccgtatcttcgtccgtactcaactcaatggccatagcggccaggggatctctctcctggtgtctccacaggatgcagagattcaggcgatccagcagttgatccgcagcaggcaatatccggtgtatgtcatgggtgttagtaatatccccggtgaagtcctcggtgctcgtctgttcacccaaggtatcgtgggccgtcgtaccgagttccgaatccacgtggttcgtggccgggctatcctgtcccaggttaagctccgccgtaacgtcgatggcgatgtggcagatgactcccaaagcatcgtacgcaacgtagcaggcggctgggtctatggtgtcaacgatgccgaagaacgacaaggccatgctcaggcagtgggcgcagcaatccaagcgatcaacttctgtggtctggacttcggtgctgtggatatcgtctacaagcacgagactgaccgtgcctacgttctggagatcaacactgctccgggtctggatgaaggcggtagcgccctggaggcatacactcaggcattcaccaacatctacgaagagggtgtaatctgatgtttgtttttgtttacggaacactgatgcacggcttcggcaaccactgtcttctggatggcgcccgattcgtcgggcatgccacctcccaagagcgaggccgtatgtactcctgtgggggattccccatcctgtccttcaaacaccgtgaggatcttgtcacgggtgaagtgtgggaactccccggtggagacgaaggaaaggaaatgttaaacaacttggatcgccttgagggttatccgggttggtacgatcgtagccccaagaatttccgtatcaacggggaaatgattaccgcccttgtctatcaccaagacgaagatcaagatctgcatatcgtcgagggtgctgactggcgtaagtttgttaataaaaggaatgctgcatgacccatactgatgtagttaaggcccacaagatttaccgtgatatccccaacctgatgaaaaccgatgatgaggggaaacgtcagctcctccgtcggatcatcggcctgatggcatcccagcgacagctacaggactccatcagtgcgtctgtagagcctgctacggaggattcggattacagcgggtactatgacgagtctgctggtattcgcttcatgtgggagcgtctcaaggccgtagaagatgagttgcgcctgctggagaaacaattgggtgaattctcctatccattcctaacgtatccgggcaatgttaacccggttccttccctggtcctcaagagtcaactggagggttaaatgaagaaaatcatcggtgatacggcttgtccgggttgccgagctaaaggtggggatcacacaggtaatcacctcatcttgttcatcgacgaagaaaaaggtagccgtttcgggaagtgcaaccgttgtggccactatgaggtactggacgaggggtttgtagtacctaagcgaacggagaagtccgaagaagatatcctccgggaaatcgctgaggtccaagagtaccccttcaaagcccttgattcccgcaagatcagtaagtccatcgcagaacgctatggggttcgcgtagggttgtccatggagaatggtgtagatgtaatcgaacactactacccacggacaagggatggtaagtaccgtgcgtataacgtccggatcttggatcctaaggcgttttactaccggggtagccccaagggtggggtcgatccgttcggccttgagacgctcatgcacaaggacgtagggcacctcaagcttattatctccgaggacgagctttcggctatgtcgattgctcagatcatggaaagtaagttacccgagaagtggaagcacctacggccttgagacgctcatgcacaaggacgtagggcacctcaagcttattatctccgaggacgagctttcggctatgtcgattgctcagatcatggaaagtaagttacccgagaagtggaagcacctacggcaggcatccattagctggtcttctggagttggctctgctggtcgagacattggtttcctcaaagagtctgggattctcgataggttccaagaggttgtctactgtcacgatgctgacgaggaaggtcgcaagtctgtcgagagggtccgtgccctccttcctgactgtaagtttgtcgaaatgcccctcaaggacgctaacgacatgcttatgcgcaaccgggcagatgaggtctatcagctcattcgattcggcagcaaggtgaagtccccagactgtgctgttaccgtagatgaggtctttgcagaagcccttgagccacctaagtggggcaagagctatccctgggaaggcctaacgaacttaactatgggtcagcgcgacggtgaaatcgttggtgtaggtggtggtactggcattggtaagactctgctggctcaccatattgcagcatggaacatcatcgaacatggggaaaacgtagggaccttccttctagaagagcaagtcgctatgacactcaagaatatcgcgggtaaggtggcccaaagggctttccaccggcctgaccttgagtgggacgtggaggcgttcaagaatgctgcgggtcagcttcggggcaagctcttcatgtggaagaacaagggtcagaatgattgggagcatatcaaggagtgcattcgctattgggcggttgctatggacgttaagaccatcatccttgacaacatgactgccatgactaaccacttgtctccctccgaaatgaacactgagattgctcgtatctccacagaattggccgggatggctgacgagttgggtatccgggtattcatcttctcccaccttaacccacccaagggcaaccgtacccacgaggaaggggctgaggtgaaggaaagccaattcactggtagccgtgcgatgcagcgttggtgccaactgatgattggtttcgagaggaacaagcaagcctctggggaagagaagcacgagagccgtatccgagtcatcaaagatcggaactatggtaacaccgggctagtgttcaccaagtataaccctgacacgggttgtctgacggagcgtgaaggaagttatgacgaagtacctgaggacgacgagtccccaatttgattacgttatctacgacctagagggtgatgccctttacgataacgtcacaaggctttggtgcgctgtccttgtggatatcccaactcaggtagtccgggggttccggcctgaggaaatggagacattctacaggatcatcgcccatgcaaagttcgtggtcgggcacaacatccttgactacgacaacagggtccttgagaaacttcatggtatcgtgattccagaagaacggtcttacgataccttggtcgcctcccgactcacttggcctgaccgtcctatgggacactcgctgggagcctggggtagattcctgaagtgtcacaagggtgacttcaacgacttctccaagttctccgaggaaatgtttgagtattgcctacaggatggggttgttagtatggcactcttcaactacctgcttaaggttctcgggatgacctgggaggagctagtagagtggaggactggttcttggctaaacgtgtgaggaactacaagcgtgaaagagaactggctatacgacgcggtgaaacgggcgttggctctaagtcaggagatgctcagcggcaccgagcccgccgtaaggtggaaaagcgtcttggacggaaactcgggtctgacgaggtggtcgatcacatcaaacgtgttaaagatggtggcggtaacggggattctaatcttcgtgtccgtagccactcttctaatgctgctgatggtggtcgtgttgggaatcgtaaggccaaaggtattcgcaagaaaaagtaaatgaggtggggccttcgggcccctgaggatctaggatgaagaagtacgtggtcaatattcagtttcaggacaccaaggtgttctatgctgacttctaccggatcgaaggagagatcattcgattcttccaaggcgaggctgactctaaccccttgctcaaccgcagcctagaagtaggcgcagtccgtggatggacttccgtagaggagaaagaagatggacaataagtacaagcctacgccccaggatgaagcccgcaaggaagcagccctagaggcagccttcagcggtggctggggtggtccagaggtcgatgctgatgacttcgagctgggtaatgcctgcgggttggaccccgaggtatgtgagtcgtgtcagtaaccctacaagtccttgtcatcttctggagtgcattctttcaggtcttcctcttggggttgaactctaagcttctcagggattacaaaattaaagctggctttgtagtctcctggtgtatcaccctcgctcagtttgcctacatcaaggcagtcgggtcgagccatttggatatcggatggttcatcttcgtgagcggttggggtggtgccctggggattaccttcgcccaatacttctatcgctggtacgacaaggttttccacaaaagggcttgacaaaagttactttctgtggtataataaccttgtggtgccgggggttgctcttatatctaaaggagacattccatgaactacctaacccatacctcttgtaacatcattgttgaaaaaaccaatgaggcacatcgtgtagagcaatttcgaaagggtcttatcaggaactacctgatgagcaccgagactcccctacaggctgccagggctatcttctccaaactccccaaaggctatctgggggttggggacttcagtggacatgagaggttggctcggtattcccccagcaagaatcgacggattgcccagaaggatcttagcatctatgatgacttctactttgatcgtcatgtgacctttgacccactggacctccttcgggatgccctcgccaatctttactactacctggacaaaggtgatgagtacagcatccgaacggagcagcgtaaggttgacttggcccgattccttgttgatgtggctatttccaagggtgtatctcaacttacctatgactggtgattatatgagtaaagtcaagagtatcctgatggagcgtattgatgagtacgtgtacaaggctgttgctgtaaagttcatggagcagcaattctccctggaccgcaaatccaccgtggattacctctcgtggctagagggcgtagatgatgaagcccttgagtcggtactcaagaaactttcggagaaatctaaggaggattaagtggactggagaaagtcactgttcgttgagcataaggtagctgatatcattagccgccagagtaaacggggggtctacttccagactcaacgggctaagtggcttatccacgtactcaacgaacgaatcctcaagattgacctagaggcagtcccccagatgcctcccatgatcgtccaagcgggagccttcagtaagccatttctaaagagtggcctgccgaacaaacgcttggtcgctctatggcaacgcttgggtcacttcgaggtatctggaccctttacagcaatcgagtacaaggactttgacctgggtaaaaccgcaaggttcaaagactggatgctgtctcaaggctggatacctgaccagtggaatatcaaggacattacagtcggcactgatggcaagaagctacgtggatccgatctgaatggtgccttgaaccgctacatcgaagacctccgaagctccccctctgggctactccgaatgaagctccagggcatcatccctgggaagactacagtcggtgaggtcaagagaaagcttgagaaacaacgtaaggttctcactacgccgaagatgactgagacctccatggataccgtccagggggatcttggtaagcttgtgatgcagcgaatggtttgggctcaccgacgctccctccttcagggattagtcgagcaggtgaagcctaatggacgcctagaggggagtgctaacccctgtgcaacacccacgggccgtatgagacaccgtgtagtagttaatatccccgctgctcgttctcccttcgggcctgaaatccgagggttgttccaggggactcctaatgctggtgaatggaaatggactgtcctccgccgcgacataggtgagaacgaaagggtaaggccccacactaacatcgtggaggtcctcaaaggtggtaagtggaagacggtaggaaagtaccgagtatacgtcccagcgaatcaactgatcttcgtgggctatgacggtgctggtctagagcttcggatgcttgcctcctacattggcgacccagactatacccgagaggttgtagatggtgacgtccatactgctaaccaaatagccgcagggctccctacccgagacgatgctaagacgttcatctacgccttcatctacggtgctggggatgccaaaattgggcagatcattggtggcacgagggccgatggggctaggctgagggagcaattccttaaggccaacccagagctggctaagctgattgagagggttaagcaggaagccgaaaggggctaccttgtaggactcgacggacgtaagctaacgatgcgccgtagtgagtctggagaggtgatggtccacaaggcattgaacaccctcctccaagctgctggtgctattgtcatgaagtgggcaatggtgatcctagatgagaaggtacgacgggctggactcaaggcgtggaaggttttggatatccacgatgaaggccagtgggagtgccatcccgaggaccttgcgactctccgtggattcatggagacttgtgtcaaggaagctggggaaatcttgggttgtaactgccccctcgccagtgattctatcgcaggtaggtcctggtatgacacacactgatacatctaggggttgacttaaggccccttttgtgctataatatcttcttccctacaagaggtatatttaatgtctaagaaagtatcccaacgattcaccttcccggtagcgaagctgatcttcccctacatcgtaactccggataccgagtacggtgaggtttaccaagttaccatctgcattccgaccaaggagcaagccgatgagctggtagccaagatggagtccaaggacgcacgcctcaaaggcaccatcaagtacaccgagcgtgacggtgaattcctgttcaaggtcaagcagaagaagcacgttgactggatgcaggatggtgagcgtaagtccgctgttatgaaaccgattgtcctgaccagcgacaacaagccgtacgatggccccaatccgtggggtggctctactggcgaagttggtatcctgatcgaaacccagaagggagctcggggtaaaggcactatcactgccctccgtctccgtggtgtacgtctccacgagattgtatctggtggtgatggtgaggatgatccgctgttcggtggtggtttcaccgaggaagaggacaagcctgaggacgtatttggcgaggacttcgatgacgaagatgctcctatctaagagcctagaggatcatgaggcgggggtatgccctttgggctgcccctactgcttagctgactatgagagaatttggggtgtacgagtggtaagttctgtagctgcatctaatgataaggtagaggtggatcccaatggaatcaaggcaggagatccgggggctaagctcgataagggcaaggtggatgttggaatcatcttcgaagcgttcccaagagctctttatgcggtggcacaagttgctaacttcggagcaagcaaatatagtcgcgggggttggagattcgtcgagaacggagtccagcgatatgatgctgcctttggacgacacctccttgagcgacacaagggtgaggttttggacccccaaagtagtttaccccaccggtatcacgaagtttggaacgccctagcagccctagagctggtaatccaacaagaggagggttctaatggaacttctgctggatccgagggctaagaccattcccagcaattactctgtcaaaggccttgaggtagacctgggggaaaccccaggctacagcctgacggatgctgctatggagaaagccaagcgtcaagagagtgagtactatgactggaagggctacgaagctctagccaaccctgtggtagaacatcccgagtatcggtccaagggtgaagcatttgccctcaccaagcattgggaagaaattcttaagaaaggtccggagggctctaaggatgatcgcggggattgatggtgacgtacttcgatacgagctaggccacatcgctatgaagaaggaacacatcttcgacattgaggtagttaggccctggcccgaggaagaagttaacgagcttgtagataaaaagatagaacaaatcatagagagagtaggcgctgatgactatgaagtgtacctcactggaaaagataatttccgattggagcttgcgaagatccgccaatataaggggactcgcgtcggaaatacaaagccacatcactggcaaacagtgtcagaccgacttaaggacaagtggggagcagtcactgtcaacggtatcgaagctgatgattggctcggggttcggggaactgaggagggggataactttacggcatgttctcgcgacaaggatatccgccaaatcaccgagtgccaccattacagttgggcctgtggcgataagcagccagaattgggaccctttcgtaacgagggccttggacgagtgtccgcttcttggagactctatgggaagaagaagccccaaaagtcctggaagcttgagggctacggtacggcattcctctatggacaactccttgttggtgactctgtggataacataccaggttgtcctgaaatcgggcctaagtcagcggcagatttgcttgggcaccttactactgagaaggatctcttccaggcttgcgcctacgagtaccaaaaggtctatggagataattggaaggagtaccttactgagaactttcgtctcctctacctcatcagagaccgtaattggtttgatattgaacagtccggtaacgagcttaattgccgagtaataagacattgggagataccttatgaagatggagaactattctattgaagaggcccttagagtatgtcgggaactcttcaagaccctagaggaaaccacaaagttcactggccttcgggttggaggcgggttcctccgggattgtgacaacggggttgtgccgaaggatattgatatctacgttgtagagccccgtaccctccaagaggtcaccatccgtaatcgggtccgtgtagttcctcaggaagtggatgaggatacccttgaggttaacgtcatccggttcttcaacgccattggccataagaaagtacggctaggggataaagaggttaataacgactatcccgattcgtttaaggtttgggagtcttgtgagttccccgagggacacctcccggttaacctgattgtatgtaagcaggagcacccctgcgtatttgacattggcctgtgctgcatcagtttttggaatggggatcgtcagttgctctactctcctgattacaccagggataagggaaataagtggcttaccctcattaacattgctgacccgttgattggccacactgttcatgagaccgttatctcagatggagcagtagaccgtcttgtgaaccacatccgaagggttatggagaaatatcctgaccataaagtcaaactctcggataaccttattgcctgggttaacctgaatggcagtactactcagcagagtgtactgaaggttatcgagagacttcaagaggagaacatcattgagcgtccaggggagattcttccggcccaagctgaagtcattgattgggacgccctacgactccaacaccgagaagctcgtgcacgaaacgatgccgtcgttggagttcaagccgaagccgctaacctacgtggtgaagcacaggtactaccccgacttcaaggcctctttagagactggctcatcgacgaagactttgctggtagaggtcaaggggtacttccaagaggcgtctgaggctgccaaatatgtatgggtgagggagagtctccccccagatacagaacttgtgttcatcttcgagaagcccgatacagcgtgccattggcttaagaagcgtaaggatggtacaaagcagactatggcggagtgggccgaacgaaatggcttccgctggttcactctagagactttcaaggagtccttccataatgagtaagcgctataatgaagataccctcgtcattgcggatacccaagtccgcccaggggtcaatgttgaccacctcgggaatctcggggaatggatcgcacgtaatcgccccaagcgaattgtgcatattggggaccattgggacatgcccagtctgtcaagctacgaccgtggtaccgccaagatcgaaggacgtcgagtcctcagcgatatccaagcgggtaatgatgcgatgcggctgctgctcaatcctcttcgccgcctacagcgtcaccaagcgagttgtaagaagagggtctaccgaccagaaatgcacttcttcatcggtaaccatgaggagcgtatcaagcggtatgagaactctaaccctgctctccaaggttttattgggtacgatcattttgatctgtccgattggattgtccatgatttcctcgacgtgggtgtcatcgaaggtgttgccttctcccactacttctataaccccaactccggtcggccttacggtggttccgccgagcatcgccttaataagatcaagcggagctttgtccaaggacacgaacaggggttcaagtaccacatcgaggcagtaggaaaggagcgtatccatgggttggtagtcggtagcttctacacccatgacgagtcctacaagggccctcagggtaacgatcactggcgaggtgtagccctccttcgtaatcacaagggtggtgagtatgacctcaagctgatgagcgtggaggagttcctgtgagtaagttcttgccagacctgtactacattaaatctgagcatgacttcggtcaacgggggttggcgtttaagacgccgatctccgcagaactctggctggatatgaagtttgggaaagggggtgctgaggaggggcttaagcgagggatgtataccatcgaagtcctggagatcctctacatccccagcgttcaccttccggatatcttggggtaatctatgaaagaccgagtgggacgtaagctagaggtaggggacagtgtagtcttcctggtccacaggaacacctcctcccatctagctattggtacagttgatggttttactccgaagatgattcgaatcaaatgccctaccatgagttggactattgattctgaatatgttctcaggagcagtgacaaggtggtgtactatgacaaaggctgaactggagaaagcacttgaagagacgcaaagcgctcttgcgaaggctgaggcgaaggccttttcctttgaagaattggctgaagaagctaaaagacagattgaattcctcgaagggatgctagatttggtagacctaagggcttctgtcttctacggggattggaggggttatgcagaaagatcaaaagggtaagggtggattcccttggacctacattgcggtagcagccttgtttgccctgctggtttatgtaggatacagctgactgatgttactcctgaccctgggagaaatatccagactcctcatcgaagtgttatcttgggcaggttccttatgaaatatcgagtgcagcagctaggtaaacgcatcttctacatacaaaggaggttcttctttacatggagaactgttaaggactcgggaggttgttttatggccttctactccttggaagatgctgaggagttcatccgggataacctaaagtatagggattcaccagtctaccaccccgtcgagtaaccaaaaaaaaaggccccaagggtatcatcccaaggggccttatccttagctccggagagcgttcagcagtgtgttgaacttatccacgagggcctcgtgagtgtccgtataggaagccacggggatctcagcgaccttacccttgacgtaagctgcaatggctgcatcaaggccactcacttcactagccttaggcttccagttacctggcttagcctcgctagccttagcacccacctccagaagctccggcttcccctcaatatcctcccacttcacaggctcaaagtctttcgggccaatgacttgtcggagggtcccatcttcattgcgtacagcaatagccttcgattgaataacgaatacactgttgccgtcgttgtcagcgatataacctgccataatctaactcctaattaaactgccgatttgaaggtacccacggtaccaccgaagggatacacacggacagtgcattccccgaattgaccatcagtctccgtctggttacctttagcgtaccaagccttatccggagatacagcaaacttgatcttagctgccgatccctggataaagtggaactgacagccaggggttacatccgggccaagggtgacggtaatatccgtggtcccctgagacatgaagaaccatccagattgttgcttagtcagggtaatattctgagtaagctgctgggtattgacgacgttaccacccaggtgagtagtcacaaggcgggggtcaaggtagttctgttggagagaaccaatgttcttcacaggaccacgcatgtcgtcaaactgctcgataatgtagtcaccgatggtaccaccgttttggtcatctggaccaatcacgtgcttgatagccacgtaaacacccaggtgttcccggcgagggtctactcggaattcactaccgaaggcagtaccgcaaggggcctcgatcatggtctcggagaagagtccaatgacgtgacctgggtctcggttaccaccagaggtctcccctggggcacccacgagggtgttaaccaggacagaggggataacagcctcggatggattatccgggaaagaagccgttacgttctctacaaccagggctgcgttctctttaactgcgtaatagtccgggaggaactcagtagcagttacgatgttagtattcactcgacccgcatccgaggtaatctcaatgagatcaccatcggcattacggcggaagacctttggcacatcaaccacgtaggcacggccaccaggaccagcggcttcagtttctacataagttgccatattacttctccttaaagatccagggcatcttccattgcttccataccggcaacgaaaggacgtgctggagtttgcttatacaagaacttccaaatctcacctgcggaagggtcatccccgaacgaggcaatagttttaccaacagccatagcaccctctgttgcagcaccagctactggccccaagagtacctctgcgggagtagttccccgacggtaacccgttagcatgtcgtagatcatagaagcctggagtggcatctgttgcatcactacgtccatcatccgttgctctggactacgggtatcctctcggctagatccaccgaacttagccagctgacgaagctcatcctggagataccccaggctcatcatcagaccaagagtgaaggctacacctgcggcacccataccagcgttggtccaagaaccagcgaagtgtgggctcagtcgtctacggaacatcggtaggatgatgttaccataggctgctgggtaacccttcaagagggagaacatctgaacgtttccgttgctcatccacataggcttatcagcgaaggtggggtcgaggactacctgatccacaaaccgacgcatagccagagtcttgacgttgttagccatcaggacttcagacggagtagctggggataccagcttaagggcatcctgctgactaccgatgttaacccccatttcccgaagctgagcaaccttcagagcaccgttggcagaactgaaggggaggcctgcggctagatccatcaggttattctgatagacacgcttggcagtctctgttgcaaatattcggttgacatgagtcaggatggacaagccgttgatgaggaactgaccacggactgccttttggatagtcgtgttaaatacctcagcaccaacccgatctgccattagagaggtagcagaggccagggtgtggttcatatcactcataaaccgaccggtctcagactttggaatcccactgtagatcctacgggctgcctgtcttactacctcccccatggttggcaatacagccccaagggttggcatcacaccagccttagcgaagggcagactgaactccgtaagggtagagaaaccagcaagtggcagtcgggagaggacaagggcaccagaggtaacagccgccagcttcttgatattaggatctttgatacgaccgtgcataccattgtaggcatccactaggtcatacatccgatccacctcttccttagtgaccctcttaccagcccgttgagcctcagctacagcagaagcaatcttagcgttagccttctccccattgataccaaaccgttcggtaaaggcaatccggtgggaagccccctcgaagtaatctcgaatttcctggagacgcttcttaggagtatcgttgagagaatacttattgaggatctcttggggcacggacccaaaggcccggctctcttccagttgaccatacttaggaactgcatcgctttgggcgaaccgtccacgtagagtatctggatcaccctggatacgataacgagggtctacttcccaagccccggtctgctggttctgagtaaccagtcggttaacctcaggggcagtgttaccacgagtatcatccgagacttcaaccagccagttagctacagcatcctcggcagcctgtcggttctggaagtacggagtaatctcattcaggaactcgggggtctgaaccttctctggggatagaccgaatggcatatagttggggatagtaccaacggacattccaccacggttaacagcctcattccttacgtcatccatcaaagcacggagacgggtagcttccggggtgttgacacctgcggatgtatcagcgataatcctatcgatctctttggaggacttaccctcaaagatgttatctagttcagaattccacttacctgcctggagttcctggtcctcaaagatagtcttaccagaggcccgcttaccactcatatcagccctgaaggtctcagagaactcacgggcgataggagaggccttggcgagtggctctagaagggacgtagcttcgtttccaagggcatcccaggccttcttgaccgtacccctaggctctagctcagacgccttaggaggggacgcaggagcattaggatcaacaatggcagagccagctgaatcctgatgacgacccagggtgtccatccccgaggatacagcaccaccggcagtacccatagcggtaccagtgaatgcagcagtcaggaggttatccatgaactgctcaggggtttgtacctgccctactgcatcataggcaatggtatcctggagcgcctgttgggcaccagatgttacaccctcggctacaccagagactgctgcatgcttaccagcttgggtaacagcctcgatagcggtttgcttaggtaggcccgattggaccaacatttggtaagcgccatctttaccgatgtgcttgaggagtggggcagcgataacaccagcacccgcagtatctagtaccgagaggccagcaccacctaggacagcagtccaggggttactaccctcggggtcaagctccttcatctggttgttcagagcacctacgttgatccccatggagctcaggaaagagcctatgagggcaccacccatacgaccaggggcaccaaacatggatccagccttagcacctgcggcaccaccagccattacggctcccatcgaggggagtgcttctgagatattattctttaggaaagaaccaatggagggaatatcttggatatcggcaaaggaccgaacatctggggttccgtactgtgacgcctcctgggcattctcctcagccatctgagtgccgtagtccttgaggtagtcactaccagtcagctcaccaacagtagcaatagtgccaccgatgttagactgcatggtgtcgatcccacgattgatcgcagaaccgatagaattaggatcagccggagcagccaaagcgctcaggtctggggcagcttcaggtacaacctccggggttacttcctggggagctacctctacttcttctggctcattcaacgaagccaactcagcggctacatcgaggtcttttacctcagccagttccgcagcaatagcggctttcatttcaggggaaagagccataagtcacctttttgctggaatagaagataagcaccttaagactatcttaagataacctaaggatttaattaataatacaataagttattaagataatcttaagtgcctatagatctattataccacagttttaataccttgtcaagtaccttttatcacttaggggcaggacgactcaaaatacctgatccccagccaaaaagacccgacggttgcttctgtagcttagccgcttcctgctgtacgaaggtaacggggtcagccccaggattctgcctcaggtggttccgaagcttctgagagatagccgactggtcttgttcaccaatcttagcaccacctaccagatccgaaccagagacacccttgaccaactcattggcatccttggtagtgatagcctcaccacgggaagccgaagcagcagccttacgtgcagcagcactgatcttggcagtcttgagacgtaccgcagaactaagctgagcattctcttgagacatatcctggccacgtctagcggtttcagcctggagctgagcccgttgggtagctaggtcttgaccacgctgggttagggagagcttggcccgttctacagcagcctgtttttcccatttagacaaaagaccctcattcttccgagaggtttcttccagggtgccccgcttaacctctagattaccaatccccagacgctcatcggatttatccttagcaaccttgcgttccaaatccttctctttaatctgacgatcagcagcagcagcagccatcttctgttggttgataccagcctggaggttgcggtcaagttgacgctcataggaagcagcgaacatatccccagccttaccagtcttgtctagggccgaagccagaagaccagtaccaatgagagcgtaagatacataacgagacaggtcatcattatccatagtcctcatctgggtgagctcttcattaacccgagtcttaagctcttcaggtttaagctctacaccttcccgttgggcatcagcctcgactacagcttgggccatttcagggcggcttacggctccactacggagaccttcgatagcaccctgttgaattacctgacggttagcctcttcttcctctgctaccgctgcaccggcctcagaggccacctcaggggctatttcaggttcgatagagggttggttaggggtaacaccataagacaacagaccggctccctgggggcctcctgtggccttctgagctgccctctggcctacaccctgggcccattggttagcactctgggcaacctcaggacccatattctctactgcctgggcagcctgagccatttcctcagcacgagtacgttcttggtcaccgatatcagcaggagttacagcagcctgaacacctacacccacaggaccaccaaggatacctgcgatacgacctagggcaccacgaccagtagcttcagccgcaggtgcaacctcttgggctgcacgggaccaacgctctgggttctccagggctgcctgagcagcacgacggacacgatcaggaatacctgagccttctacagccagcggcagtttgttccttgcgattcgcgccatatccagttcattctgagcagcccgaaggatgctagggcgaattggggacccttcataagccatgctggccataggattaacctccaaagagtttacttaggattcctgactttgggagttgtccaaagccgaggtcttctgcgctaggaagaatgcccattcccgctcccatacctgctgcaacatttgctgcgccttgttcgcttgctcccctagccccttcgacagaaaagcttggtactgcttctgaagaagactcacttccttgcccgaggagagccgcgagtgctgctccgcccattgcgccaaaagggtttgattgtccactactagattcctcactaagtcccgaccccaggtggacagggcggtaggagccttgacgactcaactgtacccgctcctgtccaccaccgatgttctgtccaatggtgaacagcttctgagttaaagaggccattagaacaacagacctccaacccgaccacctgcgttcatacccacagaagctcctgctgggcctccgaagagtgctcccagtgaggcaccacctagggcacccagggcagaaccgaggccaccaccaccgcccccagagcccttggtgacggatacgttagtaccacccatgtcacccgaaatcagttccttataggcgagaaggtcgttaaggctaacgttgttctcataggcccacttctggagagcaccattgatctcctgttgctcctgattctgtagcatagaaccagcgtcaacctgcatggcattaccagagcccagacctttagcgatagccgaaaggttacccaaggtattcagacggttctggttgtaagcctgctgatcttggaaggccagttgagaggcgttattctgttgattctggagcagcctagcggtagcaataccttcggctacacccgctcgggaactgccatactggccagcattggtcgctcctgcacgcaggtctgggcgtaccgtagtgtcgaaatcccattgcatctgctcgttggctgcaccaatggcgttcgccaagccagttttattgggatcgtaaggaccaaggtaatcagccagagagctaacacctgagctacccaggagagactggagggctcccccgagattacccaacccctcgatgccacccaattggagagcattttggtcagccactggatcaaagttcggatcaccaccgtagttcgggtcaaagcccccattatgtagccaagaactggcacccgagagtagttcattgtaattgccctgctgatatggggtagaggtggaggtagtggtttgcttcttactaccccccttataagcccgactatcaagggcatcctctacgttgaaccccatcagccgcttaacgttaaattggaggaagttcatctggagttacctctctatagaaggttacggagtcctcagtgtacccaagcttttcaagggtaggcttccagccccgacgaccttcacattggataaaccgacagcccatcttctgggcaaactgtcccaggtactcatcaacctcggggtagtctatcggagtttcattcccaggcatcttaccactccagaagaagtgtaggatgttgcccaaaggggcttgggatacttgcaataccccagcatacccactctcctcttggtagaagacaaaggcctcgtaattaaccaaggagtgtaccaggtgctcaaagtcccagaacttacccaagtcagtcctattgaatgcccgagctagtgcaggggctacagtaggaagggagtcaatattctcacgagtaatcaaatgaatcatggagtcaccactaggggataagtaaacgtacccccgaagatagcaccttggggaattccttggataagcagacgaccattagccgtaagcttaaacattgcttggttgaccacagcagtttggattgtagcccgattaagaacatcaaagacttgatccagagggggtgcattggggtctgagggtggtggataagtgatctggctttgggctggaatgatactggaaaatgctgggatgaacagttccgcaggaggccaataggcttggggaaggtccaacactgtagcaccgtcagtataattccctccagatgccagaagggtaatccacacttcatccttgtctgtattcatccggtatgcacaggtgcccatagcctgatgattattagcaggggtgaataggataaaatcccctgggagatccttgggtttagtaccggcgagtttccactggttatccaagtcataataatagatacctgatacaggacctactaccccaggggcaaaatacttaaccgttcctggcttaagcttcttagggggctccatagagaccccccaatagccatcagccacatcattgagagtctgtccaaccctaacgaattcctcattaaggaagggcagcagttcctcctcttcctgtggcggaatcgaggggctgtacctttgactcatcgcatacctgccttcggggccatttccaaagtatatccattgaagtaccagtcacctgctgaggcgaagtcaaacttcaaggcaatgtatcggcccacatgcttggtgtcaatcttatagtcctgcccaatacggtacgggtaaggccctttccatcggatacctgatccttgtacctgggcattacctacccaaatattacatactccattgcctgtaatgtgaggtataacagcacttacggtcttcatcattcggtcatccccaaggtagatatcggatctctcaagggtactcgtgaatgattgtccagagaacgtagaagtctccccgaagaggaagagcttagcatcctggaaggacgtgaagatcatactagacttcgctgggttatacgaaccttcaccccagaccgacgtatcggtatcccaggggttggagtcgtcatcccagaggttagaagtcttagggtcaatgatgccataggctccactgaggacgttggggaggtctcggatactccaagtgttctccttccagtcccaaatgatagccctatcgcaatgcttcccaggctcagatcgagtcgaggagtagcatacccacatttcagtattcacatggtcagctagcacaaaggttcgttggtagttatccgggttgatatccgagaagaagaacttacggacctgagcatcaatcacggactgcttctgtacaccgttgtggacatagacgtcaccatgacctacaacaaagtggttaccatcaaactctatagcgcagttaggaccaaggatacctacgtcattaaacagttgctggaattggaaaatgtacagaccaccaatatatcgcatggagtatacagagtcttctttgtagatgatgaaggagtcacgaagtttcacaccatccacgatagcaccattggtatcagccaaggtgttctgtccagcatctttggtagggtccgttgggtcccaagaggccgggacaccaccagcatcagctgaggtactccaccagaccatctggggcatttcaatagagttagacgttacattcaatccaatcaagaagttcttgaaggatttaagacgtctaaaggttgtattcgctgggaagttaggtagaaccctaaacacactctcagttggtggtaggtgatggggagggttaaccccatcgttagcaaagataactccattgaaggaacctacagaccaccggttggttacactcgcagagtatggacctggggacacatcaatgatggtagtcccatctgccaggtaaagcctcttttctgagcacaggagccagtatgggatgttgttccgaatgaaggggaacatatccaagataggggcctgggcagtatcaaagatgggggaatggcccagagccttctgagccttaccgttcttaaaccggacgttgttcccgaaggaccatttatccaggggcaggtcagcgggtgcaatatcagtcacgatacccgttgggttcttgacctcttgtctctctagggccattttatacctcagttcttaataatgaagaatacagcacagaacggcgggatgttgcccagtggcatgttgatcttaacagcgtgggtgtgtgcctgtccttgaccagcagggccagtctctaggttggtattggcagcgttaccagaaccaccagtcaacgcaccagaatcaccagcagaaccagtcagggtggtagcccctcgggtacgccaagtgtgagtgtgtgctgggatttgggctagcgtaagagcgtggccctcagtaagaccttcccatacgatgttagcgttaccaccccgagtacctacagtttgggatgtaccatcaataccccaagggaatgctccgatgaggttgggtacagggatgccattagaggtagcccctacgccattacaaagtttccaacctgctgggatctgagcaagagtacccgcccacatgataaccatcccaggtttcacatattgggtggtatctgcgactgagttcaattgggctgctgttacggtgacagcttggttaatattggggaaggtattcataatggcactcttgatgaggcgaatatggtcatccccaaaggacttcagatcagagccggtagggttcgtaggcaccaactgatcgatatacgttgcgacctcaagacccattcttggcctcctttaactcttctttcatttctttacgggtgacataccgctccccaaagattgccatagaaatctggagttcgctcaccgcttgggtgagcttctccgtggcctggatgttgcgttcaagcagagcctgatgcacactctgaccgacaaccgaggaacctaccgtcactaccgaggaaactaccagggcactgacgatgctgcccaagttatcagttagtagttgcatcctccgacttctcctttagattgtcctccaggactttaacaaactcgtcatcaaccttggagttagtcttctcagccagggctttagcaccagtcacgatggacttagcgattaccctggttgggaagagggtagccaggagattgattgcgagggttttgagaaagataggcattggatgctccttaacgttcgatatccttgatcgccagtcgagagctagcgaagtcagcggcattctcttcattctggagttccatgactgcccgttctagtttctgtccccagaacatcgaccgctcttcatccatggtgtacaggtaaatctgctcaaggacaccatagaggtaaatctgcgggtacttagtcagggcccaagttgttgggttagccaagctcagctctgggagaacagtccagtagttcacaatgaacggggcaccgtcaggaacaacagggtatactcgccagaagttccccaatcgagtgtagtaagttacaccctggggctggtagttgtagttaacatagtgggagaagatatcttgggtgatgtactggagagtacgtccaccgatcaaggcatcccctgtgatagatcgaagggcaacaaagtgctcagggatctcgatgcctccaccaaaggccatcagggtttcaaagtgttcgttctcacgtacccgcaggagacggttgagacggtcagtagtgttgccaatgaacaacatcagaagttcttgggtaaggtcctgacggtcagaccactggattgcggcaatagcgagatcagttacgttattaatcgtagccattcttatacccgtgcctcagaggtccgcatacgatagttgtctcggtcgttaagccaacgggtaaatcgagcggcatggtcagggtcgcaaccaatcaggttaagatcgatggggccaccctcagacattggacgattccgtagtgcctctacaacaaccagcgggatactagcaacctttcgaaggttatccttacggttagtgtttacacccgaatgacgctcttcggcgttagcggacaggattgactcaacatcttgagtatcctttcggataaagagcccaaggtcttcgtcaattgcataagtcgattggatactcatgatgatctcctataggaaaacttggggccccgaagggcccctctggattacaactgggctacgacgtcgcggatcagagcaccggacttctcgttgtttacacggaaggtgtattcaaccaggagttggcgcttctcgctgtcaccggtcttagccagctcatgctggaagaacggacgcaggtagcagaggctgtgcatcttcggatcgaagacgaacagggtgttctcgtggaaccaacggttagcacgaatggtgtacttaccgaagtcactctcatagacgtctacggtctgcgcgatgcggttatccgaagcgtccagggtgatctcagtagcacgacccttcatgttcttgctgatggctttcttgatcgagctcgaagtctggatcgagttagcctgaccaccgttacgccagatcgactccgaggcgttcaggagcatatcttcggtcagcagtcgcagatccccagcggtaccagtgttcgaaccgtcacctacaggagcaacaccgttggcacccagggagccgttggtcttgtagtaggcgaagatgttagccatctgacccggagtagtggtgttacgctggaccttagcctgaggggcaccgaccagggcgtattccatgtccagcttcagttctttggacttcttagccagttggtaagccagttcgttcttacggccagccttcttaacgcgatccgcagtaccggtgacttgcagggtctcgtcagagatttggcagtagttgttcagcatggtggtgaagctaccagccttgatggttgcatcctcaccttcgactcgggtgttcttacccggctgacggagctcgtcagtctgccattcgtgggtgatagcggtagctacacctttgccgatagccgacataaacggggtgtcgtagggggcgatgttgtagatgatatcgataaggtcttcgcgcttaccgttgatctctacagtggagacggcattagttggagttgccatataatgtacttccttctattagaaaatgtcgagagaggagaagagagcagcagcggcttcaaccgactggtctttcctcagattagcacgggcagctttaacccgcttagaaccctctgaggcttccgccctacgagctacaggcttaacagcagcaggtagttcagtctcctccttcttctctagggcagccttgcgacggacctgagactcagcccacttacgtgctgcatcaagtacagccagttggcgggcatcagcaataccccggatttcatcctcggagtagccaattgatttgccgtaagctacgatcttatccccccaggtctcatcagtagtcatttcagggatcagcttcttagccaactctgtctgacgtttgatgtaggagctgtggattatctcggctcgcttctcctgcatagccttgatatcgttacgacgtttaataagtgcctgggcttgatcccgagcctcaagagcctctaggcgaagcttctgatacttctccggatcttgagccttaagttgctcccagtttatgctatcatactgattagcaccagcaatagcggtaacagtgtactgctcaagctcggccagtagattagcacgttcaacatcaactgattcaagcttagctgcatactcttcctccagctcagcctgacgggttacaaactcttcattgcgaaggtaaccactcttgagctcttcgaagttgacctcataaatatcatccccgatcggaatctcaaagagtttctctgagaggtcatcctcagtatcagcctcaggttcatcttcggattcaccttcaggatcttcctcagactcttcctcagactcttcctcaggctcatcctcagattcaccctcgggggtttccttttcaacctcttcctcttcttcctcacctacgactttaccatctgcggtttcatcaccgggggccaggaggtcatcaccgagcaactcaccaaaggcttccgctgcctcgaattcatccatgccttgattctcaaggtccattacttatactcctttagggtgatcgaatccaagattgcctggatacgaagttgtacgcggttgagggcgtggagctcgtggtatatcgcctcccgagtctcactatctttgggggccgtagccttccattcgccctcaatctcctcttggacaatacggaaaagctcaggaagaacgttctcacgtaccatctgctgtgctgcatcagacagcactagactatagccaaaacgttctcccaagattatttcctcactgccttagtgggcttcttagtttcaggcaccttaccgtcaccgatataggcagccctcgcttgagtagcctcaaggtgatactccgcttcattacgagctcgctcccaagtgaaacgatcacgctcaagttgtagttcagcttccttaagggccatttctcgttgctggaggacagcctcctgcttcttcaactcgatttcagccagacgaatctgagcctctacctgcttcatctgagcttcagcttgcttagccatcgcatccgattgggcacgctgggcatctgcttgagctttgatatcctcaggcttaggctgggcttccttctgctctcgaattgccttggcctgtagggcctctggagagttagggttagtccagaagcgatccgggtctttgtacccagcattctctgtgacttccttaaggatgttgtaaaggttctgctcagagacaaggaccccgagaccaccacccccgactacagcctgggccatttcccagatacgcatcaggtggagcatctgctggtctttgttcatgttaccaatacctacagtaacagtcaggtcagacctctctcgccagttggcagggttaacggaaacccacttgcctcgtagctggaagacctcttcctgattctgatacttaatggcatggtcatgcagaagttggaacaaacgcttaacaccagtctctgcaaacatccgggcaatcaggtcaatctgttgctcagccgcagtcatcaactggttaacactcatagccgcttggttagagtgcagggtgttctgatctagtccgcgagtacgatcagtaataccagtacgcttaccccggtctgcctcaaggcggtcaagcatcccatagacttccccggacaactgaggagtctccagaggggtgatgctattcatggatttaactcggacgataccagccgcctcgttggtcagcaagtcttccaagttaacctgaccatccaagactacagaacgaccttggttggtacggtagatgttatccatgatgttccgcatgagtaccgaacggatctcttgaatgtcacggatcttatcgtagacactcatcccgtggaacttatgggcaatgcggtaggcattcaggtcagcgaagggacggcaatcccaaggctcgttactgataatgtaatcacctacatagaggatacgacggagttcagagataccatcaccatctacgtcaagtagggtgtagcactcagaggcccatacctcacggttagcctcagcgtcatccccagagttatactggagttgacccgtcatatcaaaattatcccggactaggcgctcaggctgactatcggagaactcatactcatcatatggaagttcttcgataacatcctcaggaacccccaagagtcggagatcacttacggtgtacttctcacggtgacagaggaatcgggcatcatcgatacaggtcgctagtctatccaccaggaagttctctggcttaacacacaggaccttgatttcccttttcttcttatccttacgaatcttaatagtgtaggtgccatcatcatctacactttgggccaggatagaggtatctgggtcagacagaatgtccgctaccatatcctcagagaggcctgagaatcgttcgaaggtgggctttagaacctcttccacgtagaccttaacaaccccagtcttcatcatgagggtgtcttggaaccagtcgaacattaccttgaacccctcattcttacgcatgaagaggtagttcacatactcagtctcttgctctgcctgctcaacatcctcggcagtatcaggctcatatttaactacttgaccaccagacgtgaacaccttcataagagaaggcataatccagtctacggtctcttggacgtccctagatacaatcgcggacttacccggacgctcattaccgaagggctcaccgaagtaatacttcagggcctcagagcgttgcttagatagttccgacgagttgaaatcaagggcgtcgtttacaagttggtctagatgacgaagtacctgttcatcatccataggcttgatcttacgacgcttagccatttagacaatactcccaaaccaatcagggatcaaccttgcggtatcactcctgtagtatccactgtttcgtacagcaccgggacgagcgtgacgggaagccatcagaagggcataccgagttgctgagatcatgtcatcattacgatccacaatcttaccatctttacggtggtacatcttcatttctttgaggaagttagtacaagtattaaataccttgagatccccattctccatacgagtaagcatccagttgacaccaaactctacagagttacccccgtgtttaccatcaggacctggggggttgctgaagggttcataaacaacattgaggttgtggtcatccttcaggaggtctacgaagcgacgacctgaggtagctccatcatgcttaaacgcatcgtgggggactacaacagggatctggtgaccacccttgaggtagatagcatcagcgtgcatgccgagggtttcgccagattcacttcgttcatcatacaggtaatacttgtccttctctgcatcccaggctacacaggcgatagcgttagggtggtcaaaccctaggtcgataccaatgatcctatggaagtgatccgggatatcaaaaggctcacatacgaacttctcttccagaatagggaagactacaccagagccgagcataggaataccctcagccctcatcctgcgttctgctggagagtataccgagagtagctgctctttaacttctggactgaggtgtggggcatcttcccagcttgcatggatcaggaactgaccaggtttaagatcctggaggaagtccttcacgatttccgtgaggccatgctctggggtaaaggttagatagacaatacccccagtagtagccgttcgggttacacactgggtataaatatccttggggcactcctcgtcaagccagatgacgtcaatagcagtacccatgaatttgtcttgggacatttcgtaggacttgaagatcaaagaggacaaacccccggatgcgtgccttaccattacagcttggacgcacccaggtttgccttccctacgttcagtctttacgatatcctctttggggatcatcccggttccgaacgcctcggggttcttccaatcacctaatagttcagactggagaatgtcccgagtagtatctgtggagatccctgcggcccagcagtttactggcttgtcaaactttcttccagtccaccactcaggataacgaccggtaagatgacaggccatgatgaaggccccggtgtatgtcttaccacagcggttgccagtcatagccagtagctgagcacaattagaagaggctgcaatgaacttctcttgccacccatagggggtgtactggttcatgcggaagtacttctgccgctcagccaactctctgactaggttacgcaaccgctcttgggtatccattaagtcaccgtgctacctatcgtagatgctcgccaaagggctcgttggaggtcccttacgcgaactggagtttgtttagcccaggcactatcctcggcttcccaagctgctctgtcgaactccccggctttcagaaggctccacgtcttggggaacttcttagtccatgctgtaccaagctggaagttaacactcactaaggcgtcgaatagttctggagtgcaaaaaggcagctcagatacttgccgttgggctgcatcgtaggctgcctgactgtcattctctagccaagtctctgctcgggctagggtgatgggtccatcctctccgggacgctgtaggtggccgtagcctccggtccaatatcccaaggagtctttgtaccactggagtactaacccctctcgcttcttaataagactcaggaagtcaagcggtcgggaaagaaaggactttagccaagtctggctcttcgtcgagtattcgtcgaacttcattcatcagatcctctgcggacatatcttccacattcttagtggtaagctccactttctgcttagcaccgaagccacctcggtccaggatatcctgggctgccttgaggcggataccgcctttctcatttggatcttctgcaatagagacgatcacacggagcgccatggggacgtgactcccgattcgctctgagataaaggcattaatgtactcggcatgtttccgatggtaagctgcaacgttacgttgagcatgattgggggagaacccagcggctacataggcttgggttttgtttaatccatcggctagtccttcgcaataaaggtctagtttttctttggcagaaagaggagcggcaccaatcgagacaaccttttcgtctgacatacgcacgctcctatctctctaatgtcaccccggtaggattcgaacctacgacctcctgcttccaaggcaggcactctaaccagactgagctacgaggagttgttggcgacccattggggattcgaaccccagacttctccctagacaggggagcactcttaccgctgagttaatgggccataatggctgggatggtagggctcgaacctacgaccggatagttaacagctatctgctctaccaactgagctacatcccaaggaaatcttaagcacccttaaggttaatttctcaaccttataatactattatatcacacttttgggtttttgtcaacccctaatttcatcttttttattattttttgctggaaaggggaatttcttaaggttcttaagactatcttaagataatctatagattaagtatttaataagtaataagtaaattaagataaatcttaagtgcctatagatctattataccacaaattgaataccctgtcaagtactttttgatatttcttgagaattcctcctgattacccgacgaacggtatggatagggttctataggtataccctaagactacccttcttatgttactggtaatggggttataaggtctattgatccgggttctataggtgtaccccaagactaccctcttgtcactataccctgggtctagttcttcttatcccagcaatataccacaaggtatagcctacgggggcctttgatccggggtatagcccctgagagtaccccaaaagtctattttatcctccggtgtatttagggaatatcc

SEQ ID NO: 8 - DNA, 65838 bp, synthetic construct
  source 1..65838: mol_type = other DNA
cgattgcccaatcctaatagaaaaaatctatcacggacgttacctatctttaaaattaataaaattaatagtaatttggtaatttggtatactttagtatttgaaagccttgcggcactaagcctgtacacttcccgtcaagtttccgattccgctcaactcgcggcagggtcgccggaaacttccagacttacaatccatgggtcgcggcaacaccacggcggactaagcggcaagtgccaaaactcgacgaacggaaccggaaatttgggagcgcgacagaatcgctcagctggacatatttctaacattcgatttaacactcaacccaaacactcaccaccatcgtctcccgccaaccgccgactcgaccctcacccaccagcagaccgcccatataacatcctataacaccacctaacactcattcaccatcaaacccacccagacctacaggccacccacaagcagcccatagacgcgatccctggccccatagtacaatcgcgccatactcagtgtcgcggcagagcaccaggggccatccgccaaccaagccaccacgacgactccagaatcgaactcagggacgcagcaccaaatgaccaaattctacagccccgacgatctagtcacgccacaggaattcgcagacccgcatttcgcggcgatcaaccaaaagcgattcgacctgtacatcgacctgcgcgtccaaggttacagctcctggcgggtcttccgggccatctggggcgaggagcatatggacggtcccgcgcaggcccgcatcttcgcgatggagtccaacccgtactatcgcaagcagttcaaggccaagttgaacgcgaccaaaacgtccgatttgtggaatccaaagacggcgctccacgaactcctccaaatggttcgtgaccccaccgtcaaggactccagccgtctgtcggccatcaaggaattgaacgttctggctgaaatcacgttcgtggacgagtcgggcaagacccggatcggtcgcggtttggccgatttctacgcatcagaagccgaggctcagaccgcgaccgtcgctgctgcggccgaagccaatggctatgtgccggaaggtgaagagggagatttcccgtctccgtctccggaaccgaccgaggaagaccgcgccaaccccatttagacataaaataacatcgttctaggcccgaatcggaccgaactaaggcgacggtagcgggaagggacgaaaaacgattctagggcggttctaggaagtcgtagcctaaccctcagaaacgacaaagccccggactctagttcagaatccggggctttcttttgggcgccttattcgccagcttcgatgatttcgaagttgtatttgacgccttcgtgctcgaaagtcattttgccagccgccttcagctgcatgcggaagcggatgtgtttcgaagaaggcaggccgaactcgatgaatgctgcgttggtggaccggaactcgccgcgcttgcctttgacagtcacagccacgccatggcgctgagtgcgtttcttggaaacttccgggtctttccaggagttggcgatggctgccgacaggtctttggactctttggctttctccggagcgttcttggcctcttcgcgcatcttccggatttcttccagcgcttcctcttcggtgatctcttcttcaggcttcagattctcttcctcggccttctcttcttccttcttcttcttggaagtgcgggttttgtagactttggccggggcttcctcttcctgaaaggcttcttcctcggccggcagagcattcaggatggcgaggcagcgacgctcagcagtcttgcggtcagagaaacgcttgacggtcgcatcggcgttgtgagcgttgtagaaggcgaccagttctttcatttctgcgttctggatgtcgccgaaggttttgatggagttggtcattttgcgatcctctgttttggaagatttcgttcgggcttcggtttgtcgccccgttgaaagagattatgcctaggtcgatgctgcgtgtctacatttatttcatcagattgatcgcaaagccgacgaacggttgtctgatgtgaaaacaccgcagagcaggttgcggtgtttggtgggcgagggcgatggtcagaaagtcgggactgtgatcggctcgattggcgggtcgccgggcctgtcgttgtccgatgcgggcgccgggctggaagggatcgcacccgaccgaagcgccagcgattccgactgcctgcttacctgcggagctggaggcgcttttggcgcatgaggcgcgggcggaaccggcaggcctggaggagccatcggcgccctgggcggctccggaagatcgggcagatcaggaagactcggcgctatgccgccaaaggaccgctgcgaccgctgcgacggattgtcgccgaacatttcggcggatcggttgtccttgatgacttgacctatggcagtcttgtcgccacccggttctgctggatcgaagcccgtgaatggagtggtaacgcagcgcatgttcggatgggtcggcagattgttcaggacgatggagtccgcccagacatacgggtttttgtggttgccgggtccgtggctgatgatctcggcaggaatatggccgttcatggtgagccattccgccatcccttgccgagtcagcgggcttaccacgatctgaccagtggactctttggcgatggcgaacagatcaccgaattcgaatccgatgattgtgtagttggacctggactcgccgacgaaagccagagtttcgatgttgtggtggatatcttggtcttcccagatgtacagcatggttcgctcctcaataaggttgcttttggtgttcgaccagagacataccggccggaactttgaagcggacgtattcgatcatcatcgccgttcgacgccgattcgtgcccgactcgtcattgacgaatagttcgcactggtgcggaatgactcgcgtcaccaggtcttcgcctttcgagccgtagaacttgatgtaggatgcgacgcctcgcgtcatgttcagacggatcgtttcgcagagtcgctgcgcggccgacgcgctagacagatcggtcggactgaccgtcacccagtaatggccactttgttctttatcggacataagctgccctccaatgagaaagcctctgcgactcggcagaggcttgttgttgttagtcgcgcttcaacagaacgactttgtcatatgcgcgatatttaccgcgccagtcttcccagtagtcgccggccgggcaggtaagctccagggtgatttcgtcgcgccagcattcgacagaaaggacaattgccttgttcctggctgcggaatccgccctgagcttaatcagaatttcgtcgccagttttcaactcatcaactcttacaacttttgccatggtgttctcctattgggcagcgccgcgagggcggatcgggtcgatgtcatgtaagcagatggtaggatagtccctggccaacttgagcttgaagtccacccagtcagcgccgatgagtgccgagacttcgatgcttactttgctgtgcggcgcagctccagatcgcaggactatggcccgacgccgaacgggatcgttgacgtcaacatgagtgagtatttcgtcgccgacgagcaactcacaggccatcaatattttggccatcatacaccctatgagaaggcgcggccaggacgatcccagccgcgccgatggattaacgtttgtgaaggatggacactgcgtccacgtcgaggatgctaatggtacggcgacggcgcggattgctgcgttcatggatgcgaatcaggttcgcagctggagctttgtccaccacccatacatcttccggcttctgcttccggagcaggaccgtcacttctgcgttctgggcgaggccgttgcagatgaaagtgaacttggacgagggagttctgtacatttcgagattcctttttggacttctggtccggcttctcagccggtgaagagattatgccctgattctcaccgccgagtaaagcatttgtgtaccaattctcccgtcaggtggaaccaaagtgcggtatcgcttatggctacgctaccgcgccatggcccttcttgctcgcacactgcgaaccacaggctgatttccatccttgccaggactcggccgaaagattcgaacctgcgccgactggacaggatgtcgacattcactcgtcgattgacctcataccggcgaccgttgatgacgaagaccagccgcagggtttgtccgtcaagacactcccagtatctggtttcatatcggagccaatagcgcttgccagccgggcctaccatggtcatttaatcgatgctcctggccgcgccacgcggtccggtcggcgggacgggatcgaaccgacccaggatgtagtcgggccggcgcgcttcctgaggacacacggcgagaaggcgccatcctgcgtccagggcagactggagttcgtccgtgcagcagtcttccttgagcagcagacggttgacgttctggagattcggaccaggtatggccgatcctgtgagtgagttccaaccctcgattccactcaccatctccggttggttgatgtagcttacggacataccggccaaccggctcgcggccagctccagacgctccagcatgggtcgcagagcagcttccgggtcaatgtcgtcccagagaaggaccaggctgatgatggtatacggatagtccttgtccagatcccaggctgaagcggtcaggcggcccaggccgatttcattacacataacgggaacgtcgttgctccaggtggacggctcccagttccgctcaccaggattcccgattgttacgccttccaggttccccaggagaacgtggagcttgctgacatattccgcctccaaggctttccgctcttcgtcggtctggttgtggcgatagaaggatggagggctgactttggcatggtagagtttcataactgttcctcggttttggaagatttgaaagttagagaatggtgtcgcagtatttctcgaaaggactctggcgcttcttctcgcagatcgcgcaggtgatttccaggtcgggcagctcgctgtaagtcttgccgagatacagccagcgcttgcacagactgcggccgtccgacgtgaagaagtggactttgcgagcattgccgggttgcgcccagccgcttggtgccggcttcctgctcataccatcaccacgtcaatttcgtcggcgaagaagttaaagccaactccatctgcgtagccagtgtaacgcagacgagtcgtcccatcaatgtgggcttcctcgacgccgatgacttccagggtggtatcgttgcagtacagcttgacgaacatcttgatgccggagccaatcgccgcgaaggcgatctgcttgtacaggtcttgcttgatcatgctttacgctcctgtttgcggatgtattctgccctgacttcggcttcgaacttcgcccagagcgcgttgtctactggaccccagggaccggtctgagttccgtccaccggcgcgaacttgccaccggcgcgacgatacttggacagcgttctcaggctgatgattttctcttcttgggaatcgaattggacgatcacttgacatacttcccgaagtagcaggtgtggagtatttccaggcctttggagatagccttcagcttgatgccgaaattcttgatcgcccagtccagatcgtggtgatagaactcgatgatgttgacgatttgaccggcgctgtagtggatgacgcgaaccttgccgttccagcggaaatcttcgcggctgtttacggcctcgactgcgatggtgtcgtttctgccgatgttccatttggagctagtcatgtcgcaatcctctttttggagtgtttcgcgttcgatgaggtaactatacctcagccacctcatcgagtaaagcattttcttcagattatctgaaaccttttgaagacaggaattgccgccagagccagtcgatgtgttcgttgcagtaacgctcgccatcggacgacatagataaggcgccatcattgatgccgaagtgggccgcagcgagacattcgaactcgaactggagcgttcctgggcgagggatatgggacatggcgtatttctgcacggtcagtgatcctcagaaggtttagcgtttgcgatagccgctactttctgcataccgaggcgatagaaatcggcttctttcctggcttttgtaagctgctccatggcgtcgcacgccgtcttgtgctgggcattggcgttcttgatgctgccttccaggctcttgtccagctcttcccggagcgccttctgcgatgctagctctgcctccaaatcctggattctgagctggagctggcgatttgtctcgccaacctggtttttcgcgggcgatggagttctgaaggcattcgtcaaggcgagtattttcgcgcaccagctgatgcttttcgactttcgcctttctgcacgtctctcgcatttctgaaagctcaatcgcatgcagatcgtcatccgggtctttacgctcttccttcgactccatccgcaggtagtactggccggctgcgaaggcgagcactgcctggtcggcatacgctgcggcatcggtcatgtatctggatttgtagcgaggcagaaacttgcactcctctggatcccagaggagactatcggcaataggccagaatttgcggaagccatcaggatcgacttctacattgaccatttgcagctggccggcctccacttcgacttccagaggaacctgaatggcgccggtgatgtactggaagtacactatgctagacataacacaatcctcataagaagtcgcggccgggaccatcccagccgcatcgtaatcactcttcgccttcgtccgcgctcagccactcttcgaaggcgaaatttaccttagactcgacccaatcttgaagctcatcggcgaactcgtcactgtcgatgtccatcggaatgcccagagtctcttcgttccacagttcggcattcagttcgaactggatagccggttcgccatccacattcagcaggatgcggtctgcgacttcatattcgtcgacatcgaagaggaaaccgccgctgatgatgtgctggatgaaagcttcgtcgaagttgctgacgccgatattgatctgcttagtcatttttttctggccccttatttggcgagtttgtactgagctttgagggtggtcagcttagctttcagggcgatcatagcctggccgcgcaggccttccatttctgcctcgctgatggcgatcttggcggagcggatttgatcgttgatctgggatttggtcatttctgcgttcctctgttttggagtgtttcgcgttcgatgaagagattatgacgctattcagaatggaagtaaagcagaattgtgaaatatttctcaaagtggacgaacggtatgccccgtcaggaatatttcctcccatggaaggcatttccgcatccgaatgttgattaggtatcccaaagcgcgcagtggcgaatgtgagtccgcacgtacatagggttgtattgtgcgaagtgctcgacggtcgcatccaaggctctgctattgccgcacccttccgctttgcgagccaggaagaatgcttctatttgctggtttgcgttcatcgtcttaccctccaggtgtttgttcgtttcgatgaagagattatgccgttggtcagaatggaagtaaagtgtattaacaataaaattatgttcaccgacgaacggttgtgctcgaccgtctgttgcggcgtcgatatactcgacctattgctgacaccggattgattagaatgtacaaactcaaccctgcactgcgagcggtctggcgaactcgcgcccgttacaaagtcatttatggcggccgggcgtcttcgaagtcacacgacgcaggcggtatcgccgtttacctcgcggccaactatagactcaagttcctctgtgctcgccagtttcagaaccgcatcagcgaatcggtctacacgttgatcaaggacaagattgagaattctgagtacaacggcgagttcattttcacgaagaactcgatcaagcacaagatgaccggatcagaattcctattctatgggatcgcccgtaacctgtcggaaatcaagtctaccgaaggcatcgacattctctggcttgaggaagctcactacctgacccaggagcagtgggaagtcatcgagccgaccatccggaaagagaactcagaaatctggatcatcttcaatccgaacgaagtgacagacttcgtgtatcagaacttcgtggtgaagccacccaaagacgccttcgtcaagatgatcaactggaacgaaaatccgtttctcagtgagacgatgctcaaggtcatccacgaagcttatgagcgcgacaaggaccaggccgagcacatttatggcgggattccgaagactggtggcgacaaatccgtcatcaatctcaagttcattcttgcggccatcgacgcccacaagaaactcggctgggagccggccggatcgaagcgcatcggcttcgacgttgcggacgacggcgaggatgcgaacgcaactacgctcatgcacggcaacgtcattatggaagtggacgaatgggacggcctggaagatgaactgctcaagtcgtccagccgcgtttacaacctggcgaagatgaaaggcgcctcggtcacttatgactccatcggtgtcggcgctcacgtcggatcgaagttcgccgagttgaacgatgccagtcccgacttcaaactgatctatgatccattcaacgcgggcggcgctgtcgataagcctgatgatatctacatgaagctgccgcacacgacgatcaagaacaaagaccacttcagcaacatcaaggcgcagaagtgggaagaagtcgcgacccgattccggaagacttatgaagcggttgagcatggaaaggtttatcccttcgacgaattgatttcgatcaactctgaaacgattcacccggacaaactaaatcaactgtgtatcgaactttcgtcgccgcgcaaagacctggacatgaacggccggttcaaagtcgagtccaagaaggatatgcgcgagaaacgcaagatcaagtcgccgaacatcgctgactcggtgattatgtcggccattctgccgatccggaagcccaaaggtttcttcgacttctaaacacagaaaagcccggatcgctccgggcttcgggtcttactcggtgtggttcctggcgctgagtgtcgacgcaacggcctcgccgactcccagggcttcctggccggccgcgagcgcttcggcttccgactcgacgatgaaatcatcaccttggccatcgcctggcggaacctcgaccagcacggcttcttcgccttcgaaccgcaggtcataggtcttttcgacggacagaccgtaacgagcgttgagcgcgtcccagagctgggcctcataggtccgcaggtcttgcagagctttctggtggctgagcatggccatgtcaacggcccgttgcagggtttcgtccaggacgttgaaccgaatgcgaagagaacgaatccgctcgacgacttccgcatccactacatgtctttcgatcattacttttcacctttgctgaatgtaacgttgtagccgttgtcggccaggtaggtcagggcgccttcgaaggaagttccgacgaggtgctttagcttcatttcgcgttgcgcggccagccagaatgcagttccggagaactcggcgcggccttcggccaagaccctgccatcaggcccggcgatccgtacatgcacggaagatagcttgatcggcatcagtgaatacctccactggcttgcgacggcatgctttcggcgcgtgcggcttcgcagtccgggcagggacatgcctggcggacgcgctccagctcgtcggcatccatgacatagagcttcccatcggcagtgtcgtgcgccatggcgatgttcgggaagtcggtggcgctcaggccggcgacagcgcggatttcgtcccaaagcaccagatgctcggcattcagtcgggcggcgagcgcttcgtgctctttagcgacgcgcgccatgaactcgtccatccggaatgcgaattccgcatcgatggcacgggccgaggccatggagctgagtcgaatcggttctttcttcatgatgattctcttttggttggcggttgttcgctgctcaggcctattacagtctgagcagcatgatgaagatgaacgcaaggaacgcaaacggcacaagtatgccggacatcagctttgacaggcgctgcatctcagcgtatttctcggttgacattccgtgctgcgccgcctcagctgcgaagcgggctttcgcctcgataacttccgggcgtagcgacaggacatattctaaggcctcttcccgcgctttttcggcctcgaccgacctagggtcgcgggcagagacttcgctgtgccctggcctcgcgggatgggcttgcagagatggaggaagttcagcagccacgactccatagtcggcgcaggcccaagcgatcccgatgatgatcgcgaggatggattggacgattcgcagcatcactttgtcgctagggaagttcataatcaatcctccaccgaccgaacgatttccatattgcgcccggcattggcccctgcattgaaggcgcgccgaccgtcgctatcgtccagacgcatgagcttcgtaacattggacttcttgtagccaggatcgccgaaatgttcgtgaaccgcagcttccttcaccacaaccagagacgttccggcagaagataccagctccatacgcttcctggttatggactgaaggcgatagctgatttcctgggtcgcggccagtttgaattgcgcggcaaccttcacgttgaaacgttcgtacccttgcgccttctgatactcccgacacagacgatcaatggcctcgaccagggagttgaacatgttcaccgccagctcaacgtccgacttgtagcctttgaagcgtacggcatgaccccagcgcttggtagtgcttccgtcgcgggcgctcctggatgccttagccgatgctctgtggttgttgatgccaccggcgaaatccatgatgcagtcattgtacgtcgccacggccactgagaaaaacttcatccagttcgggattgcggaatagtaacgagtggcaatttgctcatcgaactcttcgcgaatctcgccggtagcttcgaagtcgtgaaggtcatatttgtccttcaacttcttcacacgctcggctgcgatggccgcttcgtgcggactggaagagtcggctgccatggcggtcagtttgcggatgcggtctttcgccttctcgatggcttcaggggtgaattcgttctggtcggtcatggtcggttccttttgtctgaagggttcgcatttcaatggagctattctgccttcattcagaatggaagtaaagcactttcttccactatttcggcatcgactggaagaaattccagatccaatcacctgctaccagcaagaggatgagggcggcaaagaacaagacagcggccgcgagctgtgcgcccggcttcagtttgggatggctgagcttgggctctacgggaacggccggagcgctagcgctgcaaccggcgtctcctgggccgtagccgatgccgcgttcacacgcctggagcgacaccagggatttaagatactcggtctgcttttgcgactcttcgtagatgccggcgacggcgaaccagagggcgaacactacggacgtacacacaacccaggcgccggtcagcaggagggccagcgggccgatgacgaagacggtagcagccaggacgattgcgccgccccagatgatgaagccggccagaccattagtgatgtcgacacagaactttttcattttcagattccttcggttacgggatggaggggatttgaaactctgcgccgccgagaacatcaatgacgacttcccagagcgtcggaacagaccactggtaacggtcgaagtcggtttcaggatcgacgcccagcgtcacataggaagtggcaggccatttatgaacactgcctttccgcatgagagtcgccacgcggccgtcaccgagcggattcgtcctgtgcggaacgtacgcagacatcgaatattgctgcccggcgtcgataacaaatctttgcctctggcgcagacatacagagcccacaggattccagccgcgaccgccgcaagcctggcgagccgaagaatgctcataaccttcgtgagtcatttcgccgaggcaagccgagcgatcctcgtaatagttgatgttctccatcgatccaaccagcaccagagactcgaaatcgaatcgatgatcgtggatggccgagtgattgaagcaaagccgacgcggcagctccgggtgccaaacatggaggcgaccggccggaagttggacctgaatgaaccccaggccgtgcagagtgatcttgtccttcatcgggtcatggacggtgctcatggataatcctcagtagcagaaatgtattgtgagagttacgattgataagccggtcgcccatagaagggcgaaccaggccatggctttgatggtcgtgtagatcatccgaagaattttccggcacagatcgggccaatgcccatttcgatggatgcgtggttggtcaactcgcgaccgcagcaggagcactgaccagtcttccgaccgtaggcgactgccgattccatcggcttttcgaacatcttcagaacgtcgtcgtgctcagtatcggtgcagtcgcgactcttgatgaatttgccattggtgatccggccgaggtagatgtcgcccaggacgtacaggctaccggcgttccggctgtgagcgctagcctctttcacaacaacgatgagcggctcctcgccttcgccagctaggcggattttcgggcgcttgataccagactctttcgccttctcaaacgctttctcgatgccggaaatgtccagagtcggcgcagcagcttcctgcgcggccactttctcgcgatacttggcgaggttttcgatggcgcgcttcgcagcagcgatctgattttctgtcaaggagccgtatttatacaacgactcctgaaggctctgagcgaagctgaaggaatttccagtccaccactcgatgatgtccgggtgcgcggcttcgaaggccttaattttgaggtcgcgctcttgcgcagcgctacagattttctcgatgcgcttttctaccgccttagcacggctcttggcgcgctgctccgggctggttttatactctttgtatccgacgccgccgcaggcaaagcaggcgcgaccataagacgaagggccacggtacaggccggtgcctgcgcatttggtgcacttgtcgcgatacagcttcacttccttccgggagttcgggcgggcacccatggacacgtcttccagggtcttcggcgcttcgttgttgatctctacggtagcgaagtcatcacccaggtcttcgaagccggtgaacagattctctgctgcgttcatgtcgattctcctgtttggaaagttcgtttcgatgagttgactatacgccagaaatggaaaacggtagcgatttctcactaccgttcgtcgggtcgaagacaatcaataaatgtcgctattgatcttgaacccatgctcagcgccgtcgttgtagtcgtactcagcatagctgtcgcagtagtcgttcaaatgctgaatgatgcctagaacgtcgttcgcggccttgtgccgtttcgcggcgatggtcttggcgatgctgtcgccgacttccagggtttcggccgtccggcgatggatcagcagacggctccaaagatagagccggacgcgacgaatgatgtctcgatggcgctccagtcgtccgtgcaactcttcaatttcttgctcgcgagatttcactactcgtcgaagctgctgaacttccagttccaaatcggccttagtagccatgttcacctcaaaaaggaaaatcgtctgggactccaggaagttcgacgattattgttgagcctgatcgatccagaatgcatccgaccgagccagccctgtggggatagggtctacacgaactataacaggtaacttccatgactattcgccaggtcgatcctccacaccatctacagactggtgggcgcttcgacttcagttttcgttcgtccaagacggcgcgtctgtcgcatgaaaggcagcgcgccttaaccgacgtaaccatgggtatagtcgatcattcggatgagaccatcatcctctgattccctcgacttcagaccgcccaaagagtccgactggccgttgcatttcgcatcgatatccatcgaagacaggaatcctgccgatactgcggtgatatcaactttgtcgccggccttcttgaagtcttcgcactcagcatcgagggccacaattgcactcgcggccatgttcacatgactgatgaggtcggggaagatcacaggaacttcacgcgacatgccacggaccgtcagcttcaggactacatacttcatactcactatcccttttgtatgtgaggaaagaacttgctgttttccggatggtgaaaacgctcggtcgcaggcggtctttcttccggacattgaatcgtcgaaggcgggaaaaccgcgccggcgataatcgccgcgaggagtgcagcggatgtcgacatccagagggcggtttccaggctcacccgaacttccggacggcgcggcttcattcgctccaccctcttcctggctcataggatggtagactgccgccgcgaacccagttgtcccacatatggttcagaccagtcggcggctggggcggcggactgtagaatccgggcgtctcagtgctgctcaggaaataaggcgtgcaaacggcctggacgaccaattggtgccgctcccacatcttatcaatcgctcttagcatgacgtcttcgcactgagccttactgtcgaaccgtctgctagtatggtccggcatctggacacagccgtctccagtgcaaaggaaagcagtagcaatccatacggtgatactcgccatttcgtcaccctctttagttgatgagcagagtctattccatctgctcgccaggagtaaagcgcttttcgtcgggataaatgccgatgatgtctgcgtcgagcatccaaatgtccacggacggatcgtcgctgctgatttgatataggttgtacagctcccgtccgccgacagcacgctcgcctcgcggctcaaccgccagaaccttgccatgtccttctccgtgctcgtccaggtatatgacgtgatcaccgacttcatagctttcttttgtaacgaggcgtgaacgcgagctgttctgcgattccactacccaggaatccagcacgccgcctttcatgtcgcgaaacaatcttgccgatttgtcgcaatcgaatacgcccagaacttcgccgtctttcaacacgatatgtacgattggcaaaagagtattcatgtttaatctccattggttgataattagagtctaatctgccgaaaagttcccgtaaagaattattttctcataactgattagttgcgactgttaatctgatgtatatgtttgaatctcttttgaacgtttgatgtttcccctataataagcgcacacagccaacaaccacgtggaactacaatgtttaaactttcctggatattcgggcgcaaaaaggataacgctgcctgttctgaatcggcgccggagaaagtcgcacaaatccctcaacacgatccgctcgaccccatgataaagctgggaaggattcgcggctggaacgtcgagccggagaaagccccggtcattcgtagtgtgaaagatttcctggagccgggcctatctgtcgcaatggatagtgcgtatggtgatggaccaaccccggccgcgaaggctgctgcgggcggccagaatccctatgtcgtcccgactatgttgcaggactggtacaattcccaagggttcattggataccaagcttgcgcaatcatttcccaacactggttggtggacaaagcttgttccatgtctggggaagacgcagcacggaacggatgggaactcaaatcggacggcaggaagctatccgatgaacaaagcgcgctgatcgcccggcgcgacatggagtttcgcgtcaaagacaaccttgtcgaactcaacagattcaagaacgttttcggcgttcgcatcgcgctgttcgttgtggagtctgacgatccggactactacgagaaaccgttcaatccggatggaatcacacccggctcctacaagggaatctcccagatcgatccatactgggcaatgccgcagctcactgctggctcgacggcagatccgtcttccgaacatttctatgagccggatttctggattatcagcggcaaaaaatatcaccgcagccacctagtggtggttcgtggaccgcagccgccagatatcctgaagcctacatacatcttcggcggtatcccgctcacccagagaatctatgagcgcgtgtatgcagcggaacggacggcgaacgaagccccgctgcttgccatgtcgaagcgaaccagcaccatccacgttgacgtggagaaggccatcgcgaacgaagaggccttcaacgctcgcctggcgttctggatcgccaaccgtgataaccacggcgtgaaagttctgggaattgacgaaggcatggagcagttcgacacgaacctggccgacttcgacagcatcatcatgaaccaatatcagctggtcgcggccatcgccaagactccagccacgaagctcctcggcacttctccaaaaggattcaatgccactggtgagcacgaaacgatttcttatcacgaagaactggagtccattcaagagcacatattcgacccacttcttgaacgccactatttgcttctggcgaagtcggaagaaatcgatgtgcagctggaaatcgtctggaatcctgtagactccacgtccagccagcaacaagccgaattgaacaacaagaaagccgctaccgacgaaatctacatcaactctggcgttgtgtctccggatgaagttcgcgagcgtctgcgcgacgatccgcgttccggctacaaccgactcaccgacgatcaggcggaaaccgaaccgggcatgtctccggaaaacctggccgaattcgagaaggccggtgcacagtcggccaaggcgaaaggcgaagccgagcgagccgaagcccaggcgggcgccgtagagggcgcaggcggcccggttcccgccgctccacgcgggactaagcctctcgcgaaagcggccgaggaaggggccagcgaggccgctgaaccgccgtcgcgaccggaccccaaggccgagctgcggaacttgttggtcgatcttttgtcgaagctccaagacctggacgacattaaggcgccggacggcgtagacatagagcacaatgatgcgcctggcttaaagcgcacatccaaacctggcgtgtctggtatggagccttcggtgttttcgtccaaccgcatcgtcgggcctcgtgatcattcggaactccagagaatcaaggtgaatggaataaccaccttgatcgaaaatccgcgcggaagcattcgacaaggcaaggatgggagttggcgagttcagatgaaacaccactacgggttcatcaagggaacgaaaggcgctgatggggatgaagtcgattgcttcgtcggtccgaatctgggatccaaacgggtcttcgtcgtcaaccaagtgaacaaggaaggtcagtttgacgagcacaagtgtatgctcggcttcaacaacatcaacgatgcgaagtctggatatctgtcctgcttccgtccgggctgggatggtctcggctccatccatgaagttgacctgcccgctttccgtcgctggctggcgaacggcgacaccaccaaaccattcgggggcgagtgatggcattcaaggcctccaaaaagcgcgagcgccgggcacctcttccagtcggaagagggaagcccataattccatcggcaggaatcgaggcctggtatcgaaagcagatgaaggatatgtccaagctcatgatctccgactatcgaaacgagattgagaaggcgctgtcccagcctgcggccgaacgtttcttcgccagagacgaatccgttaacgtcctgttcaagatgactcttcgaagcctacagcagcgatggagccgcatttttgaaggtttcgcggccaagatcgccccggagttcgtcaaccggaccgaagaagccgctaccgccgcgacccttcacagcttgtcggtggccggcgtcgatcagccacgagctgcgtataatgagagcgtcaggaacaccctggaggccgcaaccacttacaatcatactctcatcaccaagatccaagaggaagtccacgagaagatttacacatcagtaatgctgtctctgacttccccgaatccggaagagcaaggaacttccggcataactaacgcacttcgcaaagtcgggaagttttctgaagatcgaatcgaactcatcgcaagagatcaaacaagcaagctttacagttctctgagcgatgagagaatggcggaaaatggagtcgaggagttcgagtggctacactcttctgccggcaagactcctcgccacacccacctggagaaagatgggaagaggttcaagctgaatgaccccagactttgggagggtccgaaagcagaccaggggccgccaggatgggcgattaactgtaggtgcagaaagataccgatcatttgatatcgataggagcgctatatgccgttagttcatggcaattccaatgaggctcgttctgaaaacatcaagcgggaaatcgaagccggtaaggacccaaagcaggctgcggccatcgcttactccatccagcgcagtgagaaagggaagacggcgaaagattgttcgcctgagctcgtcgccgatcttcgcgccctggtggactctctgtcgaggctcgtgaaatgaaccgcaagacgtgccggcgccgactcgtggtcgatgtaatcagggccaatattcacggcggattcttcagcctgaagtttgccgccatcgatttggcaatcatcggcttcgccatcttgatggcttttggccgatgatactgagaaaaatctggattctgactaaaaattctagtccggatagccgcaaattaccgtttacggaaaatagcagtaatttggaaatcctactgccgcgaggctttaacagagccagttcctaatttccgatttagccgcatgcttcaaaagtatatagcctgggaaattagaagtaacgttccaatagaattcatctataagtaacgttataatataacgtcaatctatatgctctagacgtattgaaattcaatttttaatcggtaaattggtaatttgaattagtttaggagttgaaagcctcgcggcagtaggcttagacaaatcccgtcaagtttccgagaccaaattaccggattttcgcggctgaggaaactggtaattagatcataatacaaattataatgtaagttaacagtcacggctacatctaattattgttccgcttatttacccttagatgtactgcgtatataatacagccatagtccacgactcttcgaattaacgatggcaaagtcgaaaagaaaaattgacgaaaatggatatatgaccatagagggctgcccgatcagctcttatggcgttttccaatattctgctggtcaactcggtcttccgggcgatccgacgcggattgtcaacgtatatcgcccggagtctgccgtcagcgatccggagtacatcgaatctctgaagaatctcccgttgatcgacgagcatgagatgctgtcgggattcgacgatgatgacgatagcgtggcccccgaagacaaaggggtggagggcatcatcacatccaacgcttactacgaagccccatgggcacgcggcgatatccgcatctattcccgcaacatgcagaatcagctggaaaggggcaaagaagacctgtccctaggctatagttgccgctacactgagcaacccggcatctggaacggaacaccttatgaagtcgtccaggacaagatgcgcggcaaccacattgccctggtaaaagagggtcgtgtgccgggggccagagtattggatggtctgtgttttgaccatctcagttttgatttcagaccatccgatgagggtaatgaaatgagtctcaagaaagccaagcggaagccccctgtccagcgcgtagggcaagctgctgactcggcggtcgaagagttgcgcgccctgtggccgaagctatctgcgtctgtccagaagttcctgggcgaagaagagcaggagccggagcatcaggaaggcgcagctccggccgaaccgaccgacagcgagcacctgaccgagcatccgactctggaaggtgctcagaaggatgacgaagagcaggaagaggagccttccgttgtcgatccggccgtggccgccgtcgagccggagcatcaagaaggtgccgcatccgaaatgtccggtgaaggcgaagtcgccgaactgatctctcaagtcaaggccattctggctcgactggagggcacggtagccgaaggggcggacgaagagcatggcgaaggtcaagatgtcgtcgagggcttggaagagcagagcagcctcagcggcgcgcaaaccgccagcgacgatggtggtgagggcaaggataacagcgaggaacttcctgaaatggcacagaagaacgcgcaagatgctgcaattcgcggtctctatcgcgacattgctgctaaagatcgcctctacaagcgtcttagctccgtggttggtgcgttcgatcaccgagctatggactcggctgaagtcgctgtttacggcgtgaagaagctgaatatcagctgcgcgaagggccaggaagcactggcgctcgacatgtacctgaaaggtgtcgaagccgcgcgcggcgcggccagccgtcaatcgaaagcccaggattcggccggttctgctccgcagtgcgccgagctggacagctacctgaagggggagtaactcatgttccagaaacaagtttaccgccagtacactcctggttttccgggcgatctgatcgaggacggcccgaagcgggcgcggccgggtcgaatcatgtctctgtctgccgtaaatccggctgccaccgccaccggccccaaccgcataagtcgcgctttcggttacgccggtgacgtcagctccctcggcgaaggtcagccgaagactatcgcggctcgcgcttctgaagtcgtgatcggcggcgccaacttctttggtgtcctcggtcatccgaagcactatgcgctgttcggttcggccggagactccctggctcccagctatgatctgcccgatggcgccgaaggcgagttcttcgacatggccaccggcctggtcgtcgaaattttcaacggcgccgcaaccgccctggacctggactatggtgacctggtcgcctatgtaccaaacaacctgcctaccgccgacaacgcgcttggcctgccggccggcgccctggttggcttcaaggctggctccatgccgaccggcttggtccagattcccaacgcacgcatcgttaacgccatcagtctgccggcccagtcggcggggaatctggttgctggcgttaccatcgtccagctcacgcagtaaggaggcgtcatgagccagattagcaagacccattcgcgcctcgcaggccgcaatgcgaaacctttcgacctgaaaaacatcaccaatgacgccgtggcgtctctgcgccgcatcggcctggtattcgatcacgccgtcgtccaggaccagatcaaggccttggcgaaggccggcgcgttccgctccggctcggccatggacagcaacttcaccgccccggtgaccacgccgtccatcccgactcccatccagttcctgcagacctggttgccgggcttcgtgaaggtcatgaccgctgcacggaagatcgacgaaatcatcggcatcgataccgttggctcctgggaagatcaagaaatcgtccagggcatcgtggagccggccggcactgcggtggaatacggcgaccacaccaacatcccgctgaccagctggaacgccaacttcgaacgtcgcaccatcgttcgtggcgagctgggtatgatggtgggcaccctggaagagggtcgtgcctcggccatccggctgaacagcgccgaagccaaacgccaacaggcggccatcggtctggaaatcttccgcaacgccatcggcttttatggctggcagagcggcctgggcaaccgcacctatggtttcctgaacgatcccaacctgccggcattccagaccccgccgagccagggctggtccactgccgactgggcaggcatcatcggcgacatccgcgaggccgttcgccagctgcgtattcagagccaagatcagatcgatccgaaggcggaaaagatcactctggccctggccaccagcaaggtggactacctgtcggtaaccacgccttacggcatttcggtttcggactggatcgaacagacctatccgaaaatgcggatcgtgtcggctccggaattgtccggcgtccagatgaaaaaccaagagccggaagatgctctggtgctcttcgtcgaagacgtgaacgcggccgtcgatggcagcaccgatggcggcagcgtgttcagccagctggtacagagcaagttcatcaccctgggtgtcgaaaagcgggcgaagtcgtacgtggaagacttctccaacggcaccgccggtgcgctgtgcaagcgtccgtgggctgtggtgcgctacctcggcatctaacagatgcctattcaccaaaggccgggcttccggcctttgttcactctgactctaactcggttgtaggggccggttagggcataattactaggactacgccaatgactgtttatatcgtttcctccatgactcaatcggtgtcttacaatgcgtatgacacctctgatccgtccaatcctcgcctccagcgaaagattctgattcgcggccgcgcaggcatcgcatccgaaacttccggcttcggcgacatgatttccgacgaggcaggacgtccgatctggaccccacagggcgattgcactgccgtgagcgattcggatttcgagctgcttcaggccaataagattttcatgcgtcacatggagaagggttatctgcgagtcgtgaagaccgacatcaccagtgaccaccagcggatttccaaagagactcgcaccatggagcgcgatggattccagcctctggatgctactcgcctgcagcagaaaatcaaggtgaccacagccagcgcttcccaggaacaagagttccggatttaaccgagggtttcggcatggtgattttcgacgaaaataagtttcgcacgctgtttccggagtttgctgatccagcagcttatccggacgtgcgcctgcagatgtatttcgacattgcgtgcgaattcatttctgatcgcgattcgccataccggattctcaatggcaaagccctggaagcgtgcctgtatcttctgaccgcccacctcctgtcgctttccacgatgcaagttcagggcgcggctggaggtggcgtcactgccggtgggacccaaggcggtttcatcaccagcgctacggtcggcgaggtcagcgtggccaagctcgcgcccccggccaagaatggttggcagtggtggctgtccgggacgccttacggccaagagctttgggcgctcctcagcgtcaagtctgtgggcggattctatatcggcggccttccagagcgccgaggcttccggaaggttggagggacgttctggtgatccctggcgcgaatcttctgcgaatggccttcggggtcatcggcactcaaattgtgagatatcgcaagtttgagcagcgagtgaagaatgatcaagcccagtatgtctccatgttcggggagcctttcgacctggcagcgtctgtccagcgagtccgacgcgatcagtatgcccagtttaatctggagttccaacggaattatgttatgatcttcgccaactttgatatggttgacttggatcgcaatatggccggtgaccagttcctctggactggaagagtttttcagctggagtctcaaggctcctggttttatcaggacggctggggagtttgcctggccgtggatatcggtgcggccaagctcactgatgacgggaaaccgactttctaggtggtgtatgtttgacggcgaactgatagcgaaaatggttgtcgagctgaatgcggcgatggcatctgctcaagaggctttgcagttcccggattttgaagtcgtccagaaagctcagccgacccaacaggggacgtcaaccaggccgaccatctttttccagaaactgtttgacattcctcgcggctggcctgccaccgattggtacctggacaacacggcccgcaaatatgtggaaataactcgacagcatgtagagacgactttccagatcagttcccttcattggcagaatcctgaaatcactcacgtggttacggcttctgatatcgccaactatgtgagagcttatttccaagctcgatccacaattgagcgcgtaaaggaactggacttcctcattcttcgcgtgtctcaaatttccaacgaggcattcgagaacgacaatcaccagttcgaattccacccaagttttgacatggttgtaacttacaaccaatatattcgcctgtacgaaaacgcagcatattcggccgatggggtattgataggcatatgagtctgaggcgcgattcagagctaatcgccgcgcacctccagatgttaagagccatgcgcggcaggtccgtttcagccggatggtattccacggctcgatatcctgacaaggcaggcggatcggtcggaatacaagtcgcgagaatcgcacgtctcaatgagtacggcggaactatcgaccatccgggcgggaccaggtatattagggacgccattgttcggggccggtttgttggcgttcggttcgtcagaaacgactttccgggagaaaccgaggtaactaagcctcacagaattaccatcccggctagaccgtttatgcgatatgcttggaacttgttttccgcagatcgcgccgcaatccagaaccggatagccatgaggctggccagaggacagatcactccagatcaagctcttgcccagatcggcctggcattggaaggatacatagccagaagcatcaggaccgggccatgggtggctaactcagcatctacggtcaggagaaagggattcaacagaccgctggtcgatacggcgcacatgcttcaatcgattagcagcagagtaacataaccaggagatcatccagtgatcagtcagagccgttatatccggatcatttcgggcgtaggcgcaggcgctccggtcgcaggccgaaagctgattctgcgcgtcatgactaccaacaacgtcatcccgcccggaatcgtcatcgagttcgacaacgccaacgcagtcctgtcatacttcggcgcgcagtcggaagagtatcagcgggctgcggcttatttcaagttcatcagtaaaagcgtgaattcgccgtccagcatcagcttcgctcgctgggtaaacaccgccatcgcgccgatggttgttggtgacaatctgccgaagaccatcgccgatttcgccggcttctcagcaggggttctgaccatcatggtcggcgcggccaaacagaacattaccgccatcgatacgtccgccgcgacttctatggacaacgtggcgtcgatcatccagaccgaaatccgcaagaacgctgacccgcagctggcccaggctaccgttacctggaatcagaacaccaaccagttcaccttggtcggcgccaccattggtaccggcgtcctggccgtggcgaaatctgccgacccccaggacatgtccaccgccctcggctggtccacctccaacgtcgtcaacgtcgccggccaggctgccgatcttcccgacgcggccgttgccaagagcatcaatgtcagcaacaacttcggttcgttcctgttcgccggtgcgccgctcgacaatgaccagatcaaggccgtgtcggcctggaacgcggctcagaacaaccagttcatctacacggtcgctacttccctggcgaacctcggcactcttttcaccttggtgaatggcaacgccgggaccgccctgaacgtgctgtcggcgactgctgccaacgacttcgtggagcagtgccccagcgagattctggctgccaccaactacgatgagccgggcgcttcgcaaaactacatgtactaccaattccctggccgcaacatcactgtttccgacgataccgttgcgaacaccgtcgacaagagccggggcaactacatcggcgtcacccaggccaatggccagcaactcgcgttctaccagcgcggcattctgtgcggcggtccgaccgatgcggtggacatgaacgtctatgccaacgaaatctggctgaagtcggctatcgctcaagcgctcctggacctgttcctgaacgtcaatgcggtcccggcgagcagcactggcgaggcgatgaccctggcggtgctgcagccggttctggacaaggcgacagccaacggcacgttcacctacggcaaggaaatcagcgccgtccagcagcagtacatcacccaagtcaccggtgatcgccgcgcctggcgtcaagtccaaaccctgggttactggatcaacatcaccttctccagctataccaacagcaacactggcttgaccgagtggaaggccaactacacgctgatctattcgaagggcgatgccatccgcttcgtcgaaggatcggacgtgatgatctaatggtttgcggcggactcgatccgccgctaccttccataaatggagtgaggaataagcaatgatcaacatttctgcgttcggctcgatctgccagttcacggcaagcagaactttcccgaacggattcaccgtcaccgagtttgccgacgatgcggaccccatcgacagcccgccgttcactgcggccgataccggcgtcggcctcaacggtgacatggtagtctggaaccgggcgaacatcctggaagtagtcgtcaacgtcattccgaataccgagggcgagcgcaacctggcagtcctgctggatgccaaccgcaccggaaaggacaagtcgggcgctcgtgatgtcgtcggtttggtcgtggcgatgccggacggcagcaaaatcacctgcaccaacggcaccccaatcgacggcgttctgatcaacgcggtggcaagcgtcggccgtctgaagaccaagccgtatcggttccgatttgaaaaagtgatcaaagccggtactagctgatgaagaaaattccgctgacagcagtccccaatcaagcgatctcatttaacgccggtagcagctattggaagattcgcctgtaccagaacatggacatgatgaatgccgatatcagccgcgacggcgtgatcgtttgccatggggtccgctgcttcggcgggattcctcttctccaatacagccatcagtaccgacctgactatggcaatttcgtcttcgaccgcgacgccgattggacattgttcggcgacggcatcaacctgttctatctggacggcgccgaattcgccgagtatcaggcgcttgccacgaggaaagaatgagcacatcaacgatcagaaccgggacgaacaacgatatccttttggacgacaatggaaacatggttatcctcagggatgtcgaagcgtgcgcccaggacgttcgggcggcgatgctcatgcgcaccggcgaaaacattttcgatgtgaactccggtgtgggatatttcgaatacatcttctcgccgcagaaaagctatgatgacgctcgcaaatccatcgcggatgcaattttatcctcgccggatgtgaccggcatcgagcagcttgacatcgacataaccggggaagtcttcggcgtcgatgcgaaagtcatcaccatccacgggcctgttaccacaggagtttgaaatgagtaccatccgcatccaatacgccaacggcacccaactgttcctggacggcaaaaatccgccgcccctggacccgctgccctcgtttaacccgtctgtcgaagatctggaaggcctggaccgcgaaaagaacaccgacaagggcgactcctcgccggccggtcttcccgttcccccggtaaacgtcgattccaatgtcgacaacggcggaaccatcccggctccggtatcgaccgacgctcctgcggccgaatcggccccggaaggcgcccaggaagctcctgcagcgggccaaggcgacgagaaaggcgctgaggaagccccgactaccgccccagtagaaaaggccgaggaaacggcctcgccggccgctgaagaggaagccccggctcccgccaaggccacctctcgcaaaaccaccagcaagtaaggactcgacatgatcaacgtcagcggcttcggcacaggaattgtaatagtttccgcctcatcgttcccgatggggttttccttgtcgaagttcgctgatgatgagagtcctatatcctccaaagagctggagccgttcggatatgagatgctttatgacggcggcctattcgcctttgacaaggctgctcctctggaagtatctgtatccgtcatcgcagggagcgaggatgatattaaccttcgcatccttctcaattccaaaaagggatcattccgatttcttccgggcatcatcccggatatgacgactctcgtggccactcttcctgatggcggccgcaccgttctgtccaatgggactatcatcaagggtccggccatcgacaccatacagaacaacggacgacgcaaaggcaacacgtacacttttgttttcggtagctatcttggcgcccagactgcgcgtcaagccatttctaacgttatccaatcggtactggaggtggtctgatgttggggattttcaccagcctcctaagttcgcgatctttttcgattgtggaccaaaacacaaaccagctagttgctgcggatttgaggataagccgggccaacacccggttttcttctgtagggcaacgccacatgctggaagatggtacgaccaagatggattccagaacgatccaccctatggaaatcatcgtcgaagtattttgcccttcaattgatgtcgtcgatcagatcaatcaattgctcctggatcgcgacacactgtacaaagtcatcactcgcggcatggtattcgaacggatgatgtgtaccagcgaagcgctcaatcagactccggatatgatatcggcgactcctgcgcggctgacattctcccaagttctcgtccagaatcccaagcctataatgttcagaaatgcaggggactcttctatgatcgaccgagggctggccctagctgaagacgtggttggctcggccggcgatctgttcgactacgcagtgaacggcgtccagaacgccgcagacttgttctgaggtgccaattgaactctttcctcaagtctattctcaacacgcctactctcaccatacgcgatgatgtcaccaaacttcctgtctggaagagtcttcaagtcaagaaagtggaaatttactcgccggcttccgtagtgtcgaaacctctggcgacgaaagaccagacggaagctcaagtgtacaccgaagctctggacattgatgtgaagaatggaaagatcatccaaccggtgcgactccgcatcaatgctatctgtccggacctgtccaccgttgaaagtatcatgaacgccttcaatgataacacctcgactttcgctatcacttctaagtcgatattggccgataaaatggccatcatggcgctcgatgtagatcagtctccagacatgttgaacgcggctgagatcaatatggaattcgagcaggttgagcctccagtattgaataaattcgatcctgcgttccctcaagatagtccgacttatggggtacagattcaatctctttctgatgctaatttgctggatttgggcgccatcggcgattcgatatcttcggccgcaaaatcgctatataatcgcgtgaccagctacttctgaggatgtatcatgcttgaaatcaatcttcccgatggccgtcaaactcgcgtacaaatcgaggcgtggtcggcattggatggctgggaactccagcgccgtttcgtcgaattcgctgtcagccaagattccgacttccgccgctctttcaccatggaaatcctgggctatgcgaaagtgctgcttggcgacgatgacaccggcattccgctgaccaccgcggcagtcatcaacaaccacctcggccactggaagaacgtggaactggttttcaactctgttctcaagcacaatggcatcgacccggccacgcacgccgaccggccggactattgggaacaagtcggatcgcagatggccatcgcatttctggccgaggcgtccaagctcattggtccagcaatgaagatcgccgaaggactcgccaacaagccggagtgattcatgtctagtgatttggatgaattcatacttcggtatgaggccgatactgccagagccgagcgcaatctggagcgcctccagaaccagatcaggcgcgtgaacagcgcatcgacgagtggccttcaggatttgcgccacttcgcagacggcgcggccactgaactcggccgagtcgttccgcagatcgattctgtgacgagcgcgattcgcgggatgaacgcgcaactggcgataggcgccactggcgtggccctggtcgcggccggcgtcaaggcgttcatgaacaccagggaccagtacaatcagcagcgcatccaggcgatggatatcggcatcgccccggcacgcttggaagagtaccagagaaaattcgttcgccagtccggtggaaccatcagccgcgagcagggcgcggagatgaccaaaaatctggcagacactttccggcgtgcttatcgcgatatcgggcgagtcggcccggaagcgcgaattctgcgtatggccggcgttgatgtcgggagcttccaaaagggcatgcggccgctcaacgacatcattactgatctggccacgaaaatggccaagctgaagccggacgaaatttctgcctacgctgatgcccttggcgtctcgcgagactacctgagcaccctggctaagatcggcccggccatgggaaaagtcaccgagatgacgactgcggaactccagtccagggtccagggcgagtccaacattcagaaattcaacgatgctctggcgaatctcaaccagacgttcaccaccctggaaaaccgagtcggcgaaaagctcgcgcctgcgttcaccaagctgatcgaaatcatcgacaagatagtccaggctattcccaatgaagtggaaagcttcgcgaaggacaccaaatcccgttgggaagatggagtgctcgggaaggcgaccgttggcagtgatatcctatccctcctcagccccggcgccctgctcggtcgcctggcagcgtgggggactcgtcgcggcatggaagaatcagggctcatcgacaaggacaaggttcccggcgctcaaaccagcgaagacctggccaagaaacaagaagaccaggacaaggctactaagtccatgaaagagctagagaagctggctgaccagaccacgaagtctaccaatgattttgcggtggcgatcaacatgttcagtggcgcggtatcgtcgttcgcgaatgccgttgacgagcgccaagcttgggcagcctgggcgggggaaattggtcgggcggtcggtatgggaagcaccgcgccgacttcgcgggcaaccggcgtctatccgcacgcgatctatgatcagtcgaagagtggcgcggccggacaagtcttcggcgagccaatcggcgcccagtctcttcgcaatcgcatgttctcgccgcagcgcaaggcagagccggtcaccgttccatcgtacatcaacgatatcatcaaagatgcttcgaagatgtacaacattcctgagctggacatcaagaaactcatatacaccgaaagccgattcaacgccagggccaccagcgaagccggagcgaaaggcctcatgcaactgatgccggaaatcgccaaggcgtatggtatcactgacgtatatgaccctcgccagaacatcctcggcggaacgcgcctactgcgtgaaaacctagatcgagcaaatggcgacatgcggttggccttgacatactatcatggcggactcgatccgaagaactggggcccaaggactcgcgcatatccgggtttggtaatgagcgctccaatcgagctgatggaagaggcgcagcgcaagcagaaggctgcggccatgaccgccgccaacgagacgttcgctccggaaggcggcgacatggacattcgcccctatgatggcggacgattggaaactccagaccagggcaagaaggaagatgagcgccgcgaagcccgtcgatatgacgacagggttgtgcgaccggaaattcgcatcatcgaccgcatgccagaccgcagcgacggcgaaatcctcaagatgtctaggcgtcaagaggccgaccgggcagactctggattccggaagttcccgaatcaggttcgcggcgagaccaagcagaacatccaggcccaactcactgccggagccatcgcgcaagtgatcggcgttaaccccaaccagatcatgcgccgcgaaatcagccgttctgacttgctgttcggatacaaccaagccatcctgggcaagcagcaggagatcaaggccgccgcgaccgaagccaacaacgtattcctgtctccagccaagcttgccgaagccacggccaaggtgaacgccgcatcgcgagaaatggatattctcaggacgtacggcgagcaacttctgaagagcgctccagagcgcggccaggagctgaccatcggtcggatcgacatgttggtcaacgtcaccggcgcgaattctccggaagaggctcgtgagatattcagcaagcagactgcagaccagctgactacggcaatccaggacgctcaaaacgattccgcaactaagatactctactgatgaaaaagagaattctgcgagtaacattcaacatgccttatggacccgaagtcatccgcgaagatctggatgttcgggtccggattatgaaggctgcgttgcggattcagaaccgggcgacgatggaaattttcggcctcaccactcagctgcgcgagtctcttctgtcgcagttcaccgcgtggaagcaccggcaacgtcaagtgggcagggaagatgaattgatgatcaaggtgtcggtggaagccggctactctgaccaggggcgcgagcaagtttccagagtctttgtcggcgaagttgccattgtcgatatcatttctccgccaccggacattggaatccgcatccagtgctataccaggcaaatcgacaggacgaagaccattcggaatatgccgcccgccaacacgacgtttgtcaagttcgtcgagtggggcgcgaacgaaatgggattgaacttcatctgcgataccagttacaacgatcaagttctgaagaatccgggccggtctatcactgtcgcgtcggcaatcctggcgtcgattcaggatatgtacatgccggacgtggccgcgttcgtcgatgatgacatattgatcgtgaaggaccgcgataaagtcattcgtccggatgaggtgaccaacgtcaactcgtttgttggaattccatcttggtctgaatggggcgtggaattccagtgtctgtttgagccatcgattcgtgtggctggaggcgtagcggtcgaatctctcatgaatccaagtgttaatggtaactatgtaataactgctctggaatacgatttggccagtcgggatcggccgttctatatcaaagtcatggggagtccagcagcataatggccagggaaatcaaatcattcaacatgttcggagttcactatacttcgcggcaattctctgccgtcgatgggctcagcatgatgtcggaaattcagagcgtgccgccagaagaattgctcaagggtactgatgtattggcgcatccggaagaccatccggaaggcatctggcttccattgactgctgcgaacatcaatctttatgtcgttgatcgagcgaaagtaatagctcccgtacaagttcttgcacttctgtccgaagtggtaatcgactggaactttggttttctcaaagactggaccggagtcaagattccatccagatttgtcgaagatatcaaaagcgtgaagacggcacattcaccttctgtagttgcgagcttggtcgcgaacggctcagcctctatgcgcgagttggaagagtattattcgactcaagatgcgtttaagatgatagacatcatgacggcgaagagcgtgaacgaggccttggcgtccgaagcatcgcacaacagaatcaaaaagggataattcctaaccgggcctgggaaggctatactagacctgccaaatcagaggctttcccatgtccaatatttctctaacatccgcaaaagctcccgacaggacgcgactgatcgccgctcttgacgctcggtcgcggcgggatgctctagatttcgaagtaatgataccggctcaggttgttcaatatgacagggccgagaatatcgcgacgattcaacctctcatcacctgggttgacacggaacacaacgccgtccaaaggcatcagctggttgatattcctgtaatttccatgggggctggcggcttccacataagtttcccgattcagcaaggtgacataggctggatttatgcggccgaccgcgacacgtctcagttcctggagtcgttgtcaatgtcgaagccgaacaccggccgcatccataagttcgagcatggcctgttcatcccggacgtattccgtcgatacaccatcaattccgaagattcggccgctatggtcatacaatcgacttccggagcgaccagaatttcgattcgcggcgataacatcaagataactgcgccgtcgaatgtcaccgtggatactccgcaggcgaatttcactggtgatgtgactatcgctaacactctggttgtaaacggtatcaacgtgaacaaccacggccacctcgaaaacaacccgcctgatgcccggacgaagggcggcatgattgcttaaggagaatttcatggcaagttttgatttttctgatttaacagcggggggggggtaatggctaactaggttgaaatggctaactacaactacatcgtcgatactggtgtgatagtcgcagataccgccgacgttctgagtgacgttgaagccgagttccgcgccgccctcggtgccaatatcaacttggccgcgagcactccgcagggatcgcttgtcgcggccgaggccatcgcccgttccagtgtcatgcggagtgaagcgcgaattgccaataccataaacccgaacgtttcattcggaacgttcctggacgcgatctgtgccttgatggggatcgagcgcggttctgacctgtcaaccttcggctatggagttcaagtcaccggccgcagccaaacccgcatttccaccgggtctagggtccagactccggccggcgcgatcttcacagtgatgagtgatgtcacgattcctgctggtggtgtcgcgaccatcgatatcaaatcgcaggagtatggcaacattcctctgccggtcgggaatctcatcatcatagacggaataattggctggtccggagcgaaagtcatcgcctccactcgcgtcgatccgggcagccgccaaatgagcgatgcagagttgaagaatgcccgcgttaatcgattggccatccaaggccgcaactcgactatggccatcaaggcgtatgtgagcgccgttccaaacgtcacgtcggtgaacgtaatcgagaacaacaccggcgccgttcaagtggtgaatggagtctcgtttacccttccgtatgctgtttgggtctgcgtcgccggaaacccggataaacaagcagtcgccgatgcgttgtgggccgcccataacggcggaactccatgggactatggtgcgacgaacaacggcgtcccggtggacggtccgaatggcgttcctgtgcgcgatccggcgtccggtcgaaagtatgtcgtgaagtggaccactccaattatgtacgatggatatgttaacgtaacagtccagcaaggttcctcctctgtagctccggaagccattcagaacgcagtggtcaattacgcccaggggaaagtggaaggggaagaggggctggtggtgggcgcgagcctgtcggcattcgaagtcgccggcgccatcgctcgcgaaattcccggcatctacatcaaactctgccaggtcgcgtgcgtcgcggctggctcgccggctccggctccgggcgatttcacttcggaatacgtcatgagcgcattcggccaggctaccatttccgttggtaacgttcgggtgactttcgtatgactctgcctgcgtacaattcggacatccaacaagctctgaaatggctccataaccaggcgcctggaatcaccggcctgatccagagaaaggcgcaatggtatgacagattcagccgccaattctgggccaactgggagcgcgacgttttccacttgaaaaccgccaacccgttcggccttatggtttggtgcatcatcctcggcacgccttcgaaagggttcggcctttatccaaaaaacagttcttgggcattcggtcggctacgccagaacttcatctatagcggtacacaagttccgccaccggcagacgcatcgcctggcggcaacttctacggtggcggcaatgccgaaattctcaacttggacgaaatcaggaaagtgcttcagctaagatatgtagcgctgatttctaacggctcgattgcatatgtcaatcgcatgcttcgttacatattcaatgatgatgagccatgggacgaggcgaccggtctgtacttctatctcatggactcaaccggcgagaatggccctgtggagaacttggcgatatatcggaaagattgggaaggtatggtgctattgtccagttcgcccagaacgaaccacgtgctgacatcgacccctgccagcgacgccgattggccgggagtcgatccggccgcgagcggtattccggtaacggtcgaaacggcgtccgctacggccccggacggctccgctacggtgtgcaagcttactaagccggccgggagtaccgcttacgtctccgcgccgatagatgggccgctggggtccggtagcactgtcacgttctcgttcttcgcgaaagccggctccacccgtttcattgcaattcagtcggctgccgatttccccagtcgagccgatgccgttttcgacctggattccgggaacgtaatcagcgatcagatgctggacagcagcgtggtaagcgcccgaatgattcgtctggaaaatggctggtggcgttgcgttctcacgaccaagaccgtcagttcttcgttccgcgctgcttacgtcgctccggcagaaaccaacttcagctggattgattcgagttccagcggggctattgatgtactaatttggggtgctcagatcgagcttgacgatgttcctaccggatacttggcgaccaccggaacccctgccaccatcactgattatgttctgcagaacatccaaaccggaacggtcaagttcacccagcctcttccgactggagccgaagcgtattggactggagactggaaaggtgggactgcggccgagccggccagattcgcagtaggggatgggattcaagatacattcaatttgtccagtcctgcatacatcggcctacccactagtggggcgttcaagctagaatacagagttggtccggcgcttaatttgtcgccgcaattgatcaacctcatgaatgaccgggcagttggtatcatgccgacttgcgccggttgcgatgtaaaagtcattcaggagtaatgacgtgatcacacccgaactgatacccagtccgtttgctgcgcagggcgacaaagacccgatcccgcagacttcttccactggcttcgctaaccttcgcgacggctacacgccggactacgaaatcagcctggcgtcgaacaacccgcaggccaaagcggtcgagcgaaagattcaaaaccaactcttcttcatcgcgacccagaacgcacaggcgtggcagcggcaaatggcgccgccgtggtttcagggcatgcctggcggctatgaacagaatgcagaagtcgtgcgagtcggcaatgacggcataatgcgacgttatcgttccatggtgaatgccaacgcgagcgaccctctcagcagcacgacttgggaagaacaacccgcctggtcggtgatgcgctccaacataccgatgccagctggagggccaggcctatcttctggcggagaagtcatcacgaccggccgcaacttcaatgacctgttgaatgggacgtgggagttcttctctgattcagtggtcgtcgcttctcagaacgcccccgtatatcccgcttcggctggtgccgctgctggcatgttggaggcgaaatcttgggtgtccggagccaatacgttctgcgtccaacgctacactgatcgcgtcgggaacgtcgctgtgcgcgggcttaatgccggagcgtggaccaactggatgtatgcggtaaacgtcatggcccttcaacaaggtcgggtcacctatggagtcgcggccggatcggcgaacgcttacacgttgacgctagttccgcagctccaaggcggcctggtggatggcatgattcttcgggtcaagttcaacaccatgaacaccggcgcctccaccatcaacgtctccggactcggcgccaaagccatcgtcggcgcggccaacttccctctcaccggcggcgaacttggccaaggactcatcgctgagcttgtgttcgacgcagcaggcgaccgctggaggattctggcaggcgcgccgcgcatccaagtcggcaacgccgatcaggactaccaggcgccgagctggaagcaggttaaggactatgtcgcgtcccaaaagctcaccgaggtggactgggccgatgtagtcaacaagccgaatgtcgccatccaggacaccacgccgtggttcgccaacctggaactgtccgatgctcggcctttcatcgatttccacttcaacagcaatcgcgccaaagattttgactatcggctgatatcggaagcagacggatcgctggctttctattcgcggcaggggtctgctgggcctacgcaggacatcctgttcaaccgaaattccgtgactttcttccagccgcgactcgatgtggcgaaaaacctcgcgtacatcgcgaactctgggcccctttggcagaacaccaccgccgatcagcccggttggaagttcacattcgcgcaaggcgtggacgcgaacaacaacgcggtgatcgcagtcaataccaccaatcccgacggctcctatcgctcacagataatgcgttgggactgggcgtccacgaatgtaatcttcaacaatcgtccgcttttcgccgggcaatatgttccttgggattctggtaacttcgacccttcaacaaagttgacagtcaatgccacgaaccagatcgccgggcccaccgggattcggaacaccaacggcaacaccggcaacatgaacacctggggttccggctccacgacggcttcatacggaaacgctgctcttcaaatcttcgggaaaggtggtggcgagcctgccgcgctttatttcgacaactcccagacaggatggtatctggggatggacaaggatggacagctcaagcgggccggctggtctctcggcaataacgcctacgtgattaccgacgaatccaacattcgtttccacgtgaattccatggctggcactcctgtttggggcggaaacgaattctgggggccgtggaactttaatccgaacaccaagctgaccatcaaagccggcacccaggaaactagcagcactgcgatattcagcggaactatgccattcgctccaatcgcatcgctgtccgattattctcaggcccctttgacggtttacaacgcgccaaccggtccgtctgcaaaaccggccgtcatcgcgttcattcgtcctggcaactggggagcattcttcggcatcgataccgacaacaagctgaaatggggcggcggatcactcggcaacagctccagggaaatcgccgattcaagcaacatcatgaatctttgggcggccaacccgaccgcgccgtcctggaacggccaaaccatttggcgatccgggaattttgatccggcaacgaaagtggatttgaacgccgcgaacgccaccaacggaaacatgatcttcaaccgcatcgccggaactggtagcggcatagcttcgtccggtcgagtcggtgccatcaacctgcagaacggggcgcattcggggcaagcggccgcagtcactttcgagcgtggtggaagtatcttcgtcaacttcggcttggataccgacaacgttctcaaagtgggtggtggaaacctgggggcaaacgcctacccagtcatccacgccgggaactacaacaactacatcaatcaagcgctggttcaggtcggtctggaaggcgtcggttcctatggaattttcgcggttctggattatgccgctccaaccgcaaccgttcgacccggagtggtcgtggacggttccattctcatctactcgtcttgcgccgcaaactacaatagcggtaaaagacctgccggaacttggcgctgcatgggatatgtagtcaaccgggatgccaacacccctgactccgcgacccttttccagcgagtgacgtaaaatgaaatggacgcggatcagaaacccacgttggctggacgcagtaaacatccacgccatggtgactttcgagggaatcggtgaagtgccgttcaccgccaatccgcaagacgtggaggcccacggaagggccatctacgctgcgattctatctggggagcacggacctatcgccccggtcgattcgaagcgggagaaggccttgcaggacgctatacgagccagggaaaagcgggctatccttcgggatacccgctggcccatagatcgccacgaagagcaaagacggctgggtatcgaaaccacggacggccctgggctgatcgcagcccttgttcactggaggcagcagattcgcgattggaatagcggggatcggccgcgacttcccatggctctgaaaacaatgttcaaaaatcaggagtattgatgaaaatcacgaaggatgttctgatcaccggaaccgggtgcaccacggatcgggcgatcaagtggctggatgatgtacaggcggccatggacaagttccacatcgagtcaccgcgagccatcgcggcctacctcgccaacatcggcgtcgagtccggcggactggtaagtctggtggagaatctcaactacagcgcccaagggttggccaacacctggcctcgccggtacgcagtagacccgcgagtccgcccgtatgtcccgaacgctctggcgaaccgcctggcccgtaacccggtcgccatcgccaacaacgtgtacgcggatcgcatgggtaatggatgcgagcaggatggcgatggttggaagtatcgcgggcgcggcttgatccagctgaccgggaagtcgaactattccctgttcgccgaagactccggcatggacgttctggagaaaccggagcttctggaaactccggccggcgcgtcgatgtcttctgcctggttcttctggcgcaatcgctgcatccccatggcggaatccaacaacttctccatggtcgtgaaaaccatcaacggcgccgcgccgaacgatgcgaaccatggccagctccggatcaaccgttatatgaagaccatcgccgcgatcaatcaaggctcctgatattcgcccaaaagaaaaggccgcttattcagcagcctttttgctttccggctttgcctcttcaatcttcctgacttcaaccggcgcggcggactcttcctgagtgaccgaatccacatagttccctagtgaactcagaacgccgattaacagcgctcttaccaccttgtccttgactgtctcgcctatgatcttggtcagaacggatattaactcttcccggagtctggggctgattcttggccgaaagcgcttgcgatgctctttgtgtttcatgtttagtcctctgtctgcggtcttctcctcaccccgataatggcttggggatgcgctgtgttggtcggaagggtcgggcgttattataactcgacgaaaatgctcgcgcttaactgtttaacgatacgcaccgcgatattaaatcaccttctttctggccaaggaactctggcggccgggtccggtctaaggcttaatttgtcgacattaaaacgagaaaacccggatcgcctgtaggataaggcgtccgggtttatctcgatctagtgtacgctagaatcagtggcttccgccccatccgtccagccagcaatcgaagacggcgtgtcgcggcttgtccttggcgccatgagagaaatgcttgaatcggatgacctggcccttgatggactcccggtcgttccagaaacgacgtttctcgtcgtgggtcaggctggaggccgacacgttgaaggtgacgccaggccacagacgttcgttgcggcagacgaatgcgccgaccagcccggatggcgacaggttttccgcatggctggagcgagccgtgcgacccagttcattggtgaaagattcattattgttgtgcatcagctcttctacgtcgatgatctcagcttcgtcgtagtcgtagcgcttgactttcacacagtacccttccttggccgtcgagcgaccgaacttatacaggccgtctgcacgcttgcccatggaaccttcgaatcccagctcggtgtggcggcgctcgacttcggtgaactgctcgatggaagttaccagctcctgctcgaccaggtaaattctctcacagccgatgcagttgcgcaggaagttcacgcgctcggccgccctggacaggcgctcttcggtcggtgcgcgatgatcggtgaaatcgtcgaacacgtggaaggaccagtccggttcaccactgtggcggcgaagatcgccagacgatttctggaagactttcgggtcgctgatgtcgccgcacaccagctcgccgtccaagccgttgaacaatttatcactgagatattcatagatcgactggttagtctgccgctttaggcttcgagtcaaagcctccccttcgaatgtgaaacagcgaaatccatcgatcttcggtgagaaatacatcggcatctggctttccaacagcttcgggtcaaaattcgatgcgagcatgggtttcatacagttctccagaaaagaagcccggcgaaccgggctaaatggcggtaagccggctcagatggtttcgttggcatagttcagttcggccatgatcgacgcatagcgctcgtccgactccttgatgaacacgccgtcgtacatgacgcctttgcgatccttgatggtgtcgtaggcagcagcgtagcaggcgagcatggtggtgtcatgctcttccgtcacgtcgaacagagtcatggcggccgataccaggctcttgatggccaacccatgatttccacgtgccagagcgcctgccagatcgccgaggcgctgcaaatcgtcgccgtaggtgggatgtacttcgccgctctcgatgaaggccgacagatgatcgaacagattttcgccgagttgcgcggccatgatcgtggcaaccaccattacgtcgccgatgccatctttcacatcgaccgggttgttctgaacccaggcttcgcatacttctgcgaactcttcgaccaacttcaggaattgatctttcgcagacgagcctttgatcaggttacggtcggaagcccatttaaccaccaggtcgtgaagttcgcggctcatgatttgttcgatgttcattctttcgattccttctgtatttgggatttgactgcgttgatgatggacgccgtgctctggcgcgatccgtccttggttgtgccaaaatagaaggccataacagacttcagttcggcgaaccaatagccgatgatggtgccgatggcgacagaggaagtcgggtccatcagcgcctcgcggccgaaggtgaagattgcgatgatgatgagaatggagcctgtcagaagagcgaatgtaatcgccgggcgaacgaaatcgttctgctgcgcggcaagcctcctcgccgaatccctgtctgccgcctctgcggcgaactggctaagctcggcctggagctggttctgctcagactgaaggcgattttgttcggcttgaatcgcaagctcctggagacgaacgcgctcggcgctctggagttcggcgagacgcgctagagcttccggattcgcgtctagagcgctagcgaccgaggctgggtcggccttcgaccccagcgccgtcgcgacgatagcgccaacggcggcgcctgcaggcccacccaggagcgaccccagagccggggcagccgcgccgatcttactacctatgtctttccagtccatttcgactcctcaaaagaaaggcgccattacagcgcctttctccggccggtgatttagaactcttcggcttcggtcgcatcgccgacgccaccggcgtcactgcgaggctgttcctgcttgctgtagtccaccttcacctcgccgccgacgaacgacttgtacaggtcggccgcagccttgaagtgatccgggttcttcaccaggccttccagctcgaactggacgccggaccagctgcccttgtcgttcgacatgcctacggtggtcatgcggaccaggttggcgaaagtcggcggggtgcgcaggccctgcggggtctgaacctttttctgggacagcgcggtcatgagcttcttcgaggccttgatctgcgaagacgacagggagatcagagcctggccgaaatcgccggtgtccggatcgatgacgatgacgtaatgaccacgggtgtcggcgaagtagtcagacttcttgtcgctgaccgaaccgtcttcgttcggcgcgtacaggcgtccttcgacttccttcaccttggtcgggtctttcatcatttccttgaagtcttcgacgctgatggcacccttgaaaccgccctcggcctcgcgaccggcccagcggatgaactcgcggcggtacgcggccggaatgatcagcaggccggtcttgccgtcgtaaaccttgccggtgacggtgttcaggaacatgccggccttcgcgccttcgatgtacttcgggtcatcttcatcgacctggggagacatcttctgcaaaacctggatgaaggggatggcataggactcggcatcggcgccttcgaagcctgcgccgtcatacgatcccaaatccatgaagtcgggaacttcggtagtcgcgacggcgccgccaccggtggcaactgctacggccttggtttcttcggttgcttcggaagtttcggttttcttaccagccatgttaggctccttgtttgtcgaatttcagttatcgctaactgtgggtttataataacggaagttgcaactaagtaaagcaaattacatattaagatttgctctttttcaccttcggtttcgtgatcttggcctctttgtattcgtgtacgccgatgaaatctggcagctcttcgcccttctccaggtactcgcgaccgaacgcctggagggtctggtaatggacatcgcggttgatggtggcgtcatagccggcttcgatgatggcctcggccgccttcttcgcatcttccatttcgccgcgaccgaattcggccagaaccttggtcttgatgatgccgtcgttgtcggtgtcttccagccacttccagaacttcgatttgttctcttccttgacggaaatgatggccttcggctctacttttacagtgcgaccgtccgccagagtagtggtcttctgaccaagctcctccaacagctccggaatggtgttacgcttgagggtcttcagctcctcttccttttcggccagcgccttttgaagatcgaggatttcgccgtccagctgcgaagccttgtccaccaagttcagcagtcgatggccgatgtcggtggcttcaaccgccatttcatccatgacgccaaaatagtcgatttcgcccggcgcgttctctttcaagtattccgggatttcaagctcttgctctttcatgtccgcctccaacttagtgatgttcccttacttgaaccaagtattgagtagatattatgccgcatcttccttgatacggctactgatttacatattaaatttcgtcgcgagtgctaacgtcagcctcaaacacgccatcaacgacataacttgccaggttgcgtttccactccaagctgacctggattttctcgtcgatggaatccagacagatcaggtcgaagtacagaaccgagttgacggtgccgattcgatgatttcggtcttcggactgcatccgcagctcgttgtcttcgtcggtcgtgtagtagatggccacgtctgcggctgtgagcgtgatcccgatccctgctgcggccgggttgcccaggaacacctggacgcgcttggcctggaaatcgtcgatcaatttttcccgctcggcctctttggtctcgccgtagtaggtgccaaacgaaatcccttgggcctccaaatacgccttgatctggtcgatttcctgaatgcgcatggcccagacgatgatggaacgctccgggtcttcctccagcagaccctccagaagatcagtaaacacggcgaatcgcgggttgtcttcgggcggcaggatcaccggctcaccatagacgttgatatagccggacgccacttgcttgagcttcgagcgcgctgccgcagcatcgaaagatacatcaagcatgaaatcttcgttctggataacaaagtggtagtcttcttccacgcgctggtaaacctttcgttgctcaggcgacatttcgaagtatatgcgcttgtacactttgtccggcaggaatgggagtgcttctttcttcgttacacggaaactgtgcggctcgatcagggaccgcagcttgtccaggttccggaataccgggcgcccacactcgtctttttcgaccagttgcggcggcatggtttgcttcccttccagcttgcgcatgatagcgatcattcgaggatcgtcactgggcaccaagacggaaaattcggccacaaacgcccgataggatttagtccccagaattccattccgcaagaattggaactgcatgaacaaatcggtcggtgcccgcgtcagaggagttccggaaaggatgcgacgggccacggccttctcgcccagctttacgatctttttcgcacgcttagcttgtgggtttttgatcctcgttgactcgtccacaattgcgcaaactttgaatgtatcaaggaatcgctcgacctcatcatagccagcctggtggttgattgcgtcaacgttgatggcgaagacgcgaagaactttttcatctgcgaacgtctcggaatacaggcggtccaagcgcgccctggcctttttggaagtcggtcggccgcgccaatccacgctcaaagtcttgatcgcgacgtgggtgggaatctcgcgcagaatccagttggtgtggacgcctttgggggcgacgatgagcagcgcgtccacgcgcccctgtaggaagaggcgaacggcatcggccaaagtcgtccaagtcttcccggtgccctgctccatcaggtatgcgaagttccgtttgttcagggacgctgccagggcattgaattggtgctgcatggcctcggtcttcatgcccttgacgggatatgttttggctttcatttgttctccaaatcggcgaggaactgaacaatgttatccagtccttctgcatagctcgcaacttccaccaagtcgcggctgttaagctcgaacaaatcgagcataggattcagcagcagccaatcggttccgatcttggccaggacgaagccccggccaccccagccgatccgctcccgaagaaaagggatttgcccaggttcgaaacatctggccattgggcaggtggaagcgcgtttcggccactcctccaaagccttgaactcgacccagaactggacacctcgacgattcaggcagatcgaatcggacatgccagaccgccgcgtctccagaaagtcgatcaggattcggccgagcgagcgctgcttaaacgcattcgccgctttcgtttcgcgatcattcatcaccttcgccctctttggaaactttctctgcttgcgctgccagctttgctttttcgcgttcggtcaaaatccgcttgacggccttcacgatgaacatatcgattccgctgagcttccagcctttgatgaggaaccaagaacctgtcggcgtcccttcagcaatctgctttccgtattgcagatatttttcaggccgaattctgaaacgtatcggctgatcaaccgagtcatcaacgcacatgatgtcgagaaactgcgactggcccttgtacaccgggtttttcccttgatcggccctcttcttctggcgaatcggttcgttctcatcagacagaacttttttcaccatcttgacaatgaccagaccatcatctccatcgcggatatcccgaatgttccgaatggggtttccggaagttactccaaccagctccggattgtcataagcatgaccccagagcgtgtgagcttcgttcaaatctgcgaattgaacttcagaattcgacagactcgcggcgactttctcccaatcctgaagcgtcttcaggtgggtgcctgccagctctttatattgagccttcagctccttaagatcggccttcaactccttcagatcggccttcaacagtttctccagttctttgtcttggctgattttcgccgaaagaatctgggcttccagagctgctacatcagcagccttgtcctcaacatcctgtgccgatatcgggcaattggccagggcgatcctcgcggccttgacttcctcgcgaagacgcaggaaccgctcggccttcgccgggccgaagcctttggcgttcatgatgccgccgatcaagcgtccatccgccactacccagttaagttcggaatgctccgggtccagggccgtatattctacgccttccttggccaattcgcgaagtatggacacagtttgctggtcgtctttcgccgcccgaagacacgcggccgcgtattccaggcgatgatatcgcttcatgtagcaggtccagtacgtcaccacagcgtagctgaccgagtgggagcggttgaatccccaggcgccgaacgttaccatttcctgccaaacacggtgagcatcgtccggggcgacgcctatggtcttggcgccctcgatgaacaattcccggcgcttgttgaaaaattcttcgccctttcgcgcagacatggctttccgaatcgccgacgtttgttcccagtcgaactgcccaatgtccttcacaatggacatgatctgttcttggtacaggaaaactccatatgttccagacagatactgctcgacctgcgggatggtataggtcacaggctcgcgaccggccacgcgctcgatgtatttggtggccatgcccgaagacaacgggcctggacgggcgagcgccgttatgtggtcgatgttttcgaacgcggtgatattgatcgcattggcgaccgagcggacggcctggccttcgaactggaagatgccggacatcttgtcttcgttgagaatgtccagaaccgccttgtcgttcaacggcaagtcgtacagctcttgcgccgtcacgcaattagcatcttggatgacgcccagagttcgaagcccgagcgcatcgatcttgagaagattcagatattctgaatcgggcttgtcgagctgcgcgacaccttcagaagttaccgtgcagaagtcgatcacttcatcgttgcagaccaggatgcctgccgcgtggacgccggagtgggatgggtggatttcgaggtcgcccatgcaggcggacgcaatctcatacttttcacggaagtcgcggccgggctgagttttttcgaaagtatcctccaatccttttccatatcgttcgtccgccgatgtatattcgatgatcgagtttttgatgttgtcggtgtcgtggaatggaatgccgaagcgttttccgacgtgagcgataaccgacgcggccttgagtgtgttgatgttcccaagctttaccacgttccaagtgccgtatttctgctggagatattcgaacactaaatagcgatgggtatcggcaaagtcgatatctatatcgggaagatcggaacgggaaatgtcgataaagcgctggaagagaaggcgatgcgggagcgggtcaacctcggtaatgcccaggaggtagcagaccaaagagccggctgaagagccgcgagccgggccgaccagcatatgcttcttggcgaacgcaaccagatcggcgaccaccagaaagtagctgtcgaagtctttcagctgaatctgcttgatctcttcttggaatcgatcctcatactcctgggtccattccttgatgtggccgcgactgaggcggtaggcttgcccctcgcgagccagagcgacgatatcaccatccaagtggatcatcggcgccttcgccagtttcacatccgccaactgctcaactaccgcacgagtattggtgatggcggaatcgaactcttcgcgagtcataatgtggcgtaaacggctccacaactcctcttcggtggcgatgtggcgaaggccgaccgattcccgcaccttccaggccgacgcgaaatcagcatggtcgatggacggcatgtcgttgtaagaggtaattaccaccggctttccgaatgccctggccgtctccatagcgccgtgcgcggcgaccatcgacgcgggattgatgtcaatgtagtcgattccggccagatccagatgggcataggcctcgccagcgaatttgatgacgccgtcagcttcctggaattcttgcggagtcaatccttgattctggaccgacttggaagtcaagcggtagaacttctttgtgtccttggcgagcacccaggctttgagcttcagctctttctcgccatcgtcggcgcatttgatcgggatttccatgccgaatccgcgaggcagttctgccttggtagcggcttgttcccaacggacgtggccccatgttccatcatcgacgatggcgacgaatggggattcgatctctttggcgcgctcgatgatctccgggaatcgaccatatgcggcaccgtaggagtaaccagagcgaacgcggagctgagggaaagacatcatgcggcctccattgcttgatacgcccgatacactcccatgcgcttgcagacttcgtggagcagccgcacgtcgtcgagcgcccggtgtttctgtacatatgggccgcagtagtgctcataaagatgttgcagccgcatgcggtggccgaacaacggcgccgactcttccacagtacagatatcgagcgatgggaaattgacttcgtccaggccgaacttgccgcgagccagatcgcaggtcagcatgaacttatcgaatggcaggttgtgggcaatattcgcgtcggctctggaaaagaagtcgcgaactttctggcgctgatcgaggaaagatgggtgcttgatcaggtcttcgttcttcaacccggtgatcttggtgatgatttcctcgataacgattccaggattgcagatgaactctacttcatccaaaatcttttcgccatcagttatcacgccggcgaattcgatgattctcggctgctttctcagactcaccctttggtggaacgggagtcctgtggtctcagtatcccatacagcgaatatcatgtttgttccctcttatgtcgaaaggccggctgctttcgcgaccggcctgaggagtatatcgcgacggctgaagatttacgccttctgtccgtctttcggcgtgatgcggctggagcgcatggtggcgtgaacaaacgccgaatagttgatcaagtccaaggccgaatcggcatccttgaacccgctattcgccaggcgagtgagtttacccaccatgtgcatcacgaacagggcgagtcgatgatcatcggcggtcttcgccaccaggccgttcgggaagaggatttccatgatctttccgtacatcagatcattgcgaccataggtgctctggcgggcgcggaagacttctgctgcggcgtacagattgttgagaacatcttccgcgaaatcatcgggatgggaatcatcttcgcccggccagacggattccatggcgaacggcgcggcgtcttcggtcgggtcttcggactgagccgtttcagcgagcggagacggggctttcgcaaacgcctcgtcgagggtaggggcggagttcggggcggccgggaacggctcgcctgccatgtcgttgggcgcgctatcggggtcgctatcggccgcgacggcgaagaacggcgcatcgcaaccttccaggttcaggatgtaggagtcgatccccaggctattgtaggcatcgatgatgtcctggcgatcatcgaacgccgcgacgatcttggtgacgccttcgatcttcttcaagatgtcgagcgcgactgcccgcttgaactgaggcgccggctcggtgttaccatacggtcgcatgatgagctcatactcgcgatgctcggcgatgccgagttcgcggtgaatcttggccctggtctggaaaaagtggttgtcggttcggccggtgacgaagaaaatcatgaggtcggcgtcgatggcattcctgatgcgacctacagcgtgcgggttgagagtgtccttgtcgagacgagaatgatactcgtcccattgccgttccaaggcgaagctcttgcggtggctatcgttgaatacgcagccgtccaggtcgaagatgatgatgccattctttggtttgcgttccatattcagatttccttactggttgctttctgggtgacggttttatcgatgaacgtcccatcagaagtgaggcggaaaacttcgccattctggatgaaatcgaacgattgcacgttcatcgtgatgcgaatggattcgctgccgttggtcacttcgacttgtgcgatgtcgccaactttctcgacaatgatcttcatgttacatggacttcccgttgacggccacagggttggcctcttgtttttccgatccccagaaatcgcggcgcagttcttcctgctcggccgagcgatccatccatggacgatagaacttgcactgatagatcggtttcaccggcaactcaaccacaggaatctggccgggcttcgtctccagagcggacatgaagcggtcataggattgctcatgaatctcatcttcattcagaaccttcgatccatagcgcgggaaggcacaggagccagtagcgacacagtgcggctggagcagactatcgaacatcggatagacttccagaaccagtcggcgcatttcgcggaaggcttcctgatactccccttgcgtacgaacacacaggcgaactttcgccatgtcgctcagagtgcgcaaattgaacttggccgcgatcttcgtttccatgttggaaggaatgatggcacgagcatcctggagcgatgcgccggcctccaagagcttctggtaactggtctgcgcgtcggcgatggcatcgtgccacaggcggttcaactcttcgcgggcgtgataggtcgggtccggctcgccattggccgtagcctgctcatcgaaatcccagcggaatgcttccggctgaacaacggcgctaacctccagggcgcgactggtttcctgctggtaagcaccggtccgcgtccggacgagttggtgggtaaaattcttgcttacgccctcgatctggaaaatgaagtccacgaattcgaacggcgagcgaatggtgtccagcatgtacttccagtggtcgagcttttcggcttcggtcatggtcgccgggtcttggccgcgcatgcgggtggatttcgtccccaggaggagttcccaggcgttctgggtgtaactgatcagagaaatcttcatcagaaatcttccggaattggcgtgaaggtgaatttttctgtgagagcaagtgccaggccttgggcttgctccttacgaagtccatactgctccatctcttccttaagcagttcgcaggcctccagacgagactcatactcttcagcctcatgaactgacgagaagacggttccatcagaggttctgtatacgagttcaatagccatgatcgcctcagtagcagcggatgatttcggcgcggatatctcgacggtcgaggtagtgctcacgaatcttatcgcgggcgcgctcggcctcttctcgactgccgaacgacaggttgaacgaagtgaaaggcttatcgtccaggcgcccatcgccaatcaggtacaggatgcctaccagcacgaaagacggcgctgtctgtcgcgtttgcggcgggtcgatttgcatttctaggaaagacatgggaacctcttcaggatggtctggtgagtacatcaatagcgctcctgctgagcagctacggtttccggctcgtagatgagcatgtcaacgatttccggacactggcctttcacccagtcgatggccgcgaccagtgtggtggaagcggagaaagagaaagtgcggtaagactcatggatgtacggagaccccatagaatcgcgctcggtggtgcgcgaaatgactgctttgatattaatgatccggcccatcttcggcctccactttagcgattatatctgacaggcttaacttctcgccattcaggaaataactggcgcgaaacttcctgtcgccctctggcccgacgattcgggccgttatggtgagactcccgccgccaagggcatcggtgtcccggaagaaagctagcagcgcccgcttgagcgctgcttcgcgaggatcgacggccattaacctaccacgttccagccgtgctgagcgcaccacacggcaccagcccctgcaggaaggctattcagaaggaacacaggagtcaggcggccgtcctcggtcatatggatgaagtagcgggcgccttcaccgagccactcggccttggcgatagcgcgttcgagattggccttggtggcgtaggttttggtgtggttcttgtcggtggagaaggttacttcgcgggccatttgtcgattccttttggttgaaggggttcgcgtttcgatgagggaatattacgcccacctgatccagaagtaaagcacttttgtaaattacttcacgaacattttcttggccttctgataagacgaagaagtcatcaggcgctcgatgacgtccatgtccgaaaccagatcatccagaaggacgttgcgccaggtagcgaaccggccgagcgagaagatgccagcctcatgggtgagattccagatcatggattcgcgctcgtcgcgaccgagcggaatgattttgcctttggtctggatggtcggctcgccatcttcgatgagctgtttccttctgatgccgaaggccgcgcaaacgtaatccatatcccagtcgctgtcccattcgatggtttcgattttaccatctaaagtctccactatccctttagtgatggattcgacgatcagagtatcaccggtgatggacgctcgaaacgttcccactttaggaccagggaaatacacggtctggaagacatcacaggggatggagagcttgtatcgactcacgatgatggaggttccttcaccgaatgacgggtcgattcccaggtccagccctgccgcagccagattggcgcggaacggcgcggtgctgatgatattcacgtggtcatcttgccggcgaagatactggaagaaagaggcgtcgaaaggacggctccaagtgatacgattcgcaagcttagccaccagctgctcatagtagtcggccggtgcgatccaacgcttttcagtcgccagattccagatggaccggtccgacaggccgcccgttactttcctggagtacatgttgcagtggtcgatgcgcggctgggaaatgaactcgccgtcgatgtagatagctttgtgtacagtgacttcgcggaacgggatgccggtgagttggccaatcactggcgagcggaatcgcagaagtgcgttgtggcgctccttgttctccggcgtcgccgcgtcgatgatttgggcttgaggaaagcgatgcgcggcgatcagtccggcgagtccggcacctacgatgattactttctgatcaggaatcatgatgtgtcccttatgagtgtacaaaacttgagaggataaaaaagggacccattttcatgagtcccttgaagagctagacgattcaggctcagaggagcggcggcttactcctcctcaccttcggaaccatcggcgccctgatcttcaccgccgtgctcctggccttcgtcatcggccttctcgtcatcgccctggccagcttcgtcttccttcgaagcgatggcaaccagatcgacccagcccatgatttccagcttgctcaggtagctgcggaccgaggtgccatagagaagatgggccaccttatcgccgaaggattcgatttcgaccggctcaccaacggtgcagtgctcgttgatgtatgcgaacaccttgccgcgagtcgagaaggcctgcggagtgccatggccgtcgccggtcgggatgaagtgagtggcgcgggggcgacgcgagccgttggacttcaggtcttcgcggcgggcttcggccttcttgcggcgctcttcctgctcttccttgcggcgctgcttctcggcttcgcgctcagccttcttctgctcggccaggcgcttgcgctcttcttcgcgagctactttctgagcttcctgggcggccttcttctcttcggcctttttggcgcgctcggcttccttctcggccttcttctgctcgcgctcggcttccttcgccttcgccttctcggcctgctcggcttccttcgccttggccttttcagcgcgctcggcttccttggctgcggccttttccttcgccttctcggcgcgctcggcttctttcttctcgcgctcggccttacgcttctcttcgcgctcagccttcttctgttcggcttccttggccttggcctcggccttctcggcgcgctcgcgctctttacgttgacgctcagcggccttctcggccttgcgcagggcggctgcttgttccttggtcagctcttcgccttgggtctgctcgttctggtctttctgttccatgttcttactccgggaatgtttaaatggatggcttattggcctgtgagaggattatctctaaactaattgaagaagggaatacctttcgcctgaacttctctaaatatttttctttcgggaaagtccagactccagggaacttatttatgttagcgaagtttttaactcttacgcaaaaacaacaagtattcaattgcgcgagttatgccagtatacatcaactggctgtaaggaattgaaggcaagttttcttccaacattgcgacccgcttccattccgatccctgcgacttgtggaacgtcatcgcccagccgaagtcgaatccgccaatggccttctgtgcctccagccgcacatcttcctcaaccgaaaagctaagaggattgaacttcacccagcgctcatagttcgtgccgataatgcgaactttggcaaacagcatttcatccggctcgtcatcatcttcttggccttcggggaccggcttgaagtccagcagaattgcttgctcgccgttcatgatgccatattcgtgctggttcccggtgcacaccagcttctcgccgatttccggttgtacacccttgtagccgaggatgcggcgcgccctggcgttcaaacggcggcgagtattgttgtaggcgcaaaggatcacgccatcatcgtccaagaacgtccgcatttcgtcgtccgacatgtcgaatccggcccgtaccagaatgtcgtcatactcgcggcagggtaggcgcttgccctggcggacgaacatcgacgcccgaacgatattgccggcgttgcgctcgatttcggtcatgatggtgtcacagctgttctcatggaaaatctggacgccgcgcacaggaggaacttggccaaagtcaccaatctccagaaccggaattcggtgcgacagcaagcgctcttcatcccactcgccgatcatggacgactcgtcgagcacgaccaacttcggcttctcgtcgagcgagtctttgttggcaaacatgatttcgccgtcttcatcttcaccaatcggccgatagatgaagctgtggagggtccgggcattggtgcaacccttctcgcgaagccgcgctgctgctttcccggttggcgcgacgaagactgtccagtccatcgagcagcaaagttcggcgattatcttcgcgatggaagtcttaccagttccggcgaaaccagcgagtcgatagacctggcggcggtgcgctcgatcacaccaaccgcgataccagtccacaacggaattgatcgcgtcgatctgctgactgtttggtcgaaagccgaatcgctcttcgatctgatcgacggtgaagttagatgctgacatatttacgttctccaacgctaggtttaatggaatcgaggctcagtttaagcacgccgtccacagaccagccagtgctacgacgatacttgcggccttgcggatcgacatagaagttcttcgtgcgccgcagcagaacataatgccaagcagctccaagcgcgtggacccttcctttatacgggaaagccttcgcctgctctgcggcctctttggccccagggagccagcggacggtcgaaaggaccaggaccgcccccttaatagcctgggaagcggcgcggccgtcctgtgggctatagcgatgtctatcgaagtctacccagtggtggttgccgcgccggagcttgaccgttcgggatcgcccctcgaacaccaccgtgccttcgtgagtaaaaatatgttccgccatcgaatgttcctttataacgtacagttatgctttacctctgcgcaggaagagtatactatcagctgactcgccaaagcgagcgaatttaatccaactttacttcggcaggaaagtggccgatactagcgccgccgcctgtactgccctccaaaacagaggatacattaaatgcaagaatgcaagatttcccgcgaccaactcccggtcggtaatccgaatcccaatgtcgacaagacccgcgacccgaaccttaagcccggtttcctgcgtcgcagccgcgagctggacccggcgctggccgttcgcatccgtcgcgagctgatccacgccgaagcatccgacttggccaaggccggatgggtcaattcacagtccagcctttatggatcgaaagccttcccgcgccattccgtcgttcgcgtgaccggagttccggaagatggagctttcatcggcatgctgatcggcttcatcgagcatcgcgagcatggcgaatgggcggtcatggaagccggaacgaaagaaggcggcgccgtcattattccagtcgatcacatcatgcgagcgtcattcgccgaagccgaagagttcgccgaaaagtgggagcggaacttggggtggcgtctcctgcgccaactccgcgagtgcggcgccctggccgggactgaagacgagttcctgcggcggatcatcaatcgatacgttcgtgatcgcacgatcctcgatcaccacaaagtcggcgcggacaaaacctatactgatgcagtactcaaaagcatcggcgaaacatggccgaagattccttcggggaaattcgtcggacaccgagtcgcgcagctcctgatcggccacaagctaggtcgagcggggaccatcctgaatgacctggtggacttcctggagaagttcgcggccgggcgcgataaagttctcaacatcgccatctgtaattgaggtgaatggcatgtataataaaccgactttgaatcaccaccatcaaaccgcattgttatatctgtataacaatcccgatcagccggccttcacaggccccaacaatcaggcgctaaatgaacttcggcagatggggtatgtaaaggcgaaaaaattcgaaaactgggcaggcaccggccatcttaggatggaatggacgctcaccaaggccgggattgagcgcgtcgaagtcgggtttctggggaagtgcgccgcttgcaagggaatcggtcagacgttgcttcgggggaagtgtactgtttgcaatggccgcggccaggggtggatcagcgaatggtcgcagaagccaatcgaagataatcctcaaattgcccctaagttcgaaaagaccgatgcgaataggcttgcagacgccattgaagaaatcggccggctggaaaaggcactggccgaatccgaaaagcgcgggagcgagctggccgcgagctattgtgacggcgtagtcggcgatgaatacggccatccttattgtcgttataaagtggaacgcgacgccgccttggccagggtcgcggagctggaagggaagttgacggactgggtgcacgaagggttccggctcaacgaggcgctagcggtcgccaaggctcaacccagcgtgccggaaatatctggcatcggtcgcgatgccgaacatcccagagctgtagtgctgtatctgcgtaacgaacccagcgatgaagatatgcgagcaattcagaactttttgcgcgccatggccgcccccgcacagcacagcgtgccgaaagcatggctcgacgttcaggcagagcgccgccggcaggtcgaggcccaggcgctaaggcaggaagtcgcagcactgcgcgcaagggcggatgttgtgccggaggtgttcgggcttgagcggtaccgcgtagaaaaaactgggaagggtttctggccatactgcgtgcgtgcgggggatggaacgcgtgaactgttcgtcggccacttgaagcagtgtcagcgggtagcgaccgaattggctacggcgtttgaagatgggaagtttatcgccgcgcccggcagctcggctcagcacagcgtgccggaggggtggaagctcgtaccgatggacccgacatcgcagatgacctatgtcggtcagtccctgcgttatgacgcggtaaacagcatcggcgagatttaccggcaaatgctcgccgtagctccctcacccatcgatccggccgcgcatcctcagccgtgccagcaaccacaggcccatcctgcccgctgcgggtgcgagcggtaagtgccaagtaaggaattcatgtaatggaacagaagaaaccttcaccagtagatggagtcatcatgaccagcctcgacgttctccggaaagcacagcccgaagcccaggacgagtatgccgtgtccatgttcgcaacggcgatccgccagaaaatgcagcgctcccgcgataaaggccgaggcggctggatcgactgcgacgaagatattctgatcaacggattcgccgaacatgcgctgaagggcaatgagaacaacctcctggacctggcgacgttcctgatgttcatgtgggtgcgcggcatcgatgatgcgaagattcccccggcgctcgaaaaggcgcggcagcacaagatcatggaagcatggagccgaatccatgaagatggcctaaactccgccagaaaggcgagcgctgcgcgacagtttgtgggagtgccacgacgcaaggggcgcccggagcgacttgcatgaagcctcacgaaataagactggcccaggccgaagagttcctgcgcgaactcggccgagggattccggacgacgaacgggtaatggtcggctacgctgaagaggccacggtccaaaccgacgagaacggccgcaagctcaacgccggctggtggcctgtgccatggaaggaagggaaatacatcaactcccgatccaacgcctacgcatgtatctcgtcgtcaatcaagacgccgaacccgaagaccggccagatgcgatactggcgcggcgaagcctctttcggccacggcctggcgttgatggtcgatgacatcggctcaggcaaagggtccaaaggcgacttcgaccgtgatgagttccgcgagcgactggagccgactgcgattgtggagacttcgccgaacaactaccagttctggtatttcttcaaagagccgatggcccacaggctccagttcaaggcgctgctctattcgttcgtggaccaggtgctaaagaaaggcggcgacaacaccgtcaaggacgtcagccgttacggccggatgccattcggcttcaacaacaagcgcgggaaagacggcaagttcaaatatgccgacgaaaacggcaagccagaactcgtgcgcctgtacagcgccgactattccaagcgctactcgccggaagagatcgcccaggcattcggcgtccgcatcattatgccacagatgaagaaggtggagataaaccgcgacgattgggtgtatgaccaagtatggctgaagtatgccgagcacatctgcacgaaatacaagatgggagaagcggcaggcggccaggtccagcagaatatgtccggcaaataccgcatccgctgtccatggggcgacgagcacaccaacggcgatcctttcggagcatactttcgcggaccgatacctggcgccgagcacgaatatgtgttcggttgcgggcacgatacttgccgcaaagagcatcgccggacttgggcggccttcacggatgaagtcgtgctaccctatatcgtcgaacaattggaaagaatcaaccgccgtcatatcggtgaggagtagacaatatgcaaaacgatcctggaatcctgattaccgccatcggcttgctgttcctcggccttatcatcttcttcgaaggcctaaagggatggaaaatacaagtcgcaaacttcctcgcgtcgcttctgtgcttcttcttcggcctttctgctttgacgttctggttcgtcgttgcgtttgacgtattttaatcgacgaacggtacagaaattttcggatggggacggaacttattagctatgccggtttaggtaggagataatagccgtccctttcgcctcaatgtgtagaggcaatgttgaatccgatcatgtaaagcagaaggcggcaaacctaacatgattatcgacgaagataatatttttgatgatggcgaatcagggtccggtgagtttgatctcacgcagatagaagatgctggaatggaccctttgatgaccgccgcgagcaaggcggccgatgatgcgattgcgaggaacgaaacgcatcgcgcgcaaaaggcggcaagatacgccgaggcgtatgctgaaccagacttgaggaagcgagcccgattgttgatgctcgaccaggcctttgatcttccggtcagccggttggtgaaagggccgtttgatgacttcatcacgaaatacagctcgacttccgacagcaactacctcgcggtctacgacacgttgttctgtaagggcgacgggacagtcccgcatccgcacttcgatgagtttcgcggccggctggtggaccatcgcggcgtggcgttcaacaacaagaccctcgatccgattgacctgatgggcgccctcgcggctgcggccttggacgatccctcgattaagaagacgattgagacttgctgcgtttgggcgcgtcgatatcgccgcaactcgctgatcgagacgtttgagaagaagataccggattgggacggcgaagagcggatcgaaacgctgctgatcgacttgttcaaaccgttcgacaccgagctcaaccgtatggtgagcaagtatttctggctgagcctgtactgccgcatcaactatccgggaatttcggcgccgatctcgttggcgttgattggtgggcaggatgcggggaaatcatatttcggcttgttgatctgtaaggagctgtccggtgggcgcgatctggctccagttcagctcgacctgagccgacacgaccagacgccgttcctgcgaaacatcaccggcaactcggttattgcgaacgtcggggaaatgtccggcttcaaaaagggcgacatggaacgcatcaaagaattcctggtgcgatcttcggatacattcgatcagaagtttgagcctggcgaaacgatcaagcgacaatggatcaccatcatggacggcaacggatacgatgggctccagcgggacgactccggcaaccgtcgattctatccgatgttcgtggcacaacttcccgacgaagatggaaagccgaactgggttaagccgggcgatggtaatgaaccgttcaaggtggacttcaccgacttcggccgcaaattctggcaagcgatggctgaatgccgagcatggatcgaagagcatggtgtcgatggatacttgaatatggtgtctgaagcgaaccgcgaagtgcagaacttctctatttcggaaatggagaatgcgcgtggcgtagttcgtgacgatacgattgacatgtatctgatcaatgttctgatcagttgcgagttcgaagagatcaagcctggcaggaatgccaagaatcctggatggagggctgacaccgtcgccattctgaaatggttcgatattctcgccaggaagaagccgatttctcgtcaccttactccgcacctgaaggcgctgggattcatcccgaataagaacggcctggctggttggtgcttacctgtcgataaggttgcgcctggatggacgaatggcatgcacacgacgttgccgccgttcaatgatgcgcttgtgtacttgctgagaaagggcgacccggatatcactgatgaggctgccatggcaaaaattcgggcagtccgggccgagcgagccaagattttgggcgaggatttctgatgtagtggtggagtgtacttggactaggccgccttcgggcggtcttttctttggtgcggcgtacattcgattccggtggtggccgagtgagattcgaaagctatccggtaattagggtggggtggcgacggtgtattgtggaaagttcgtggacgttggatcgaggattttgtgtggtgagtggatgaattgtggaaaatcgccgattctgactggatttttggggtcggccgcgtcgagcttaccggtaatttacgaaaccggtaattagggcctagccttagtgccgcgtgggtttgaagggcttaaacggatactttccggtttcggatgcagccgcaaaactattctacagcgaaaat

(19) United States
(12) Patent Application Publication
(10) Pub. No.: US 2025/0170195 A1
(43) Pub. Date: May 29, 2025

(54) BACTERIOPHAGE COMPOSITIONS FOR TREATING PSEUDOMONAS INFECTIONS

(71) Applicant: Armata Pharmaceuticals, Inc., Marina del Rey, CA (US)

(72) Inventors: Sandra P. MORALES, Sydney (AU); Gillian MEARNS, Sydney (AU); Deborah A. RANKIN, Sydney (AU)

(21) Appl. No.: 18/922,927

(22) Filed: Oct. 22, 2024

Related U.S. Application Data
(63) Continuation of application No. 16/959,658, filed on Jul. 1, 2020, now abandoned.
(60) Provisional application No. 62/731,774, filed on Sep. 14, 2018.
(60) Provisional application No. 62/678,600, filed on May 31, 2018.
(60) Provisional application No. 62/613,049, filed on Jan. 2, 2018.

(51) Int. Cl. A61K 35/76; A61K 9/00; A61K 45/06; A61P 31/04; C12N 7/00

(52) U.S. Cl. CPC A61K 35/76; A61K 9/0019; A61K 9/0078; A61K 45/06; A61P 31/04; C12N 7/00

(57) ABSTRACT

The present disclosure relates to compositions of bacteriophages, and use of the same for medical and non-medical applications, in particular treatment of Pseudomonas infection.

### CROSS-REFERENCED APPLICATIONS

This application is a continuation application of U.S. application Ser. No. 16/959,658 filed Jul. 1, 2020, which is a national phase application of PCT/US19/12113 filed Jan. 2, 2019, which claims priority to U.S. Provisional Application No. 62/613,049 filed Jan. 2, 2018, U.S. Provisional Application No. 62/678,600 filed May 31, 2018, and U.S. Provisional Application No. 62/731,774 filed Sep. 14, 2018, the entirety of which are incorporated by reference herein.

### SEQUENCE LISTING

The instant application contains a Sequence Listing, which has been submitted electronically in xml format and is hereby incorporated by reference in its entirety. Said xml copy, created on Oct. 21, 2024 is named 2024-10-21 Sequence_Listing_ST26 054249-501C01 US.xml and is 474,987 bytes in size.

### BACKGROUND

The present disclosure relates to bacteriophage, compositions of bacteriophage, and use of the same for medical and non-medical applications.

The widespread use of antibacterial agents, typically in the form of small-molecule (chemical) antibiotics, such as penicillin or tetracycline, has led to a huge increase in antibiotic-resistant bacterial strains. Mutations conferring antibiotic resistance, or genes encoding antibiotic resistance enzymes are becoming increasingly common in pathogenic bacteria worldwide.

Pseudomonas aeruginosa is a serious opportunistic bacterial pathogen. Infections caused by Pseudomonas aeruginosa include bacteremia, pneumonia, otitis externa and otitis media along with other ear infections and other topical infections of humans including Pseudomonas keratitis and Pseudomonas folliculitis; infection of burns and skin grafts in humans; hospital-acquired infections; and lung infection in cystic fibrosis (CF) patients. Pseudomonas aeruginosa is notorious for its resistance to antibiotics, and therefore infections caused by P. aeruginosa can be difficult to treat.

There remains a need to provide alternative therapeutics to small-molecule antibiotics, and to tackle the problem of antibiotic resistance in pathogenic bacteria strains.

### SUMMARY

Provided herein are compositions and methods useful in the treatment of bacterial infection. Provided herein are bacteriophage, including non-naturally occurring bacteriophage, and compositions of the same. The compositions can include one or more obligately lytic bacteriophages and optionally a cryoprotectant. In some aspects, the bacteriophage includes a nucleic acid sequence, or has a genome, including a nucleotide sequence, having at least 76%% identity to any one of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. Such variant bacteriophage can have lytic ability against Pseudomonas. In some embodiments the sequence does not have 100% sequence identity to one or more SEQ ID NOS: 1-8, but has between 76% and 99.9% sequence identity. In some cases, each individual bacteriophage is not prone to generalized transduction and/or does not carry antibiotic resistance genes. In some embodiments, one or more of the bacteriophage are not naturally occurring. The bacteriophage and compositions as described herein have a broad host spectrum and are capable of targeting a wide range of clinically-relevant Pseudomonas isolates. Moreover, the compositions as described herein have been shown to be particularly efficacious in treating bacterial infections (e.g., lung infection, bacteremia, and other Pseudomonas infections). The bacteriophage composition can be an alternative to conventional antibacterial agents/therapeutics, and overcomes one or more problems associated therewith. In some aspects, the bacteriophage composition can be utilized as co-treatment or in combination with conventional antibacterial agents/therapeutics.

In an aspect, provided herein are bacteriophage compositions that include one or more bacteriophages selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), and bacteriophage, including mutants, having 76%-100% identity (and all sub values and sub ranges therein, inclusive of endpoints) to any one of SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8. In some cases, one or more or all of the individual bacteriophage is not prone to generalized transduction and/or does not carry antibiotic resistance genes. In some embodiments, the composition's target bacteria range is broader than the range of any individual bacteriophage in the composition or the sum of the individual bacteriophage in the composition. In some cases, each individual bacteriophage is not prone to generalized transduction and/or does not carry antibiotic resistance genes. In some embodiments, one or more of the bacteriophage are not naturally occurring. The composition can be substantially free of bacterial components such as bacterial endotoxin, bacterial host protein, and the like.

In an aspect, provided herein are bacteriophage compositions. The compositions can include one or more obligately lytic bacteriophage that infect and lyse Pseudomonas. The bacteriophage can have or include a polynucleotide sequence selected from: SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8, and bacteriophage or mutants having at least 76% identity to any one of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In some cases, each individual bacteriophage is not prone to generalized transduction and/or does not carry antibiotic resistance genes. In some embodiments, the composition's target bacteria range is broader than the range of any individual bacteriophage in the composition or the sum of the individual bacteriophage in the composition. In some cases, each individual bacteriophage is not prone to generalized transduction and/or does not carry antibiotic resistance genes. In some embodiments, one or more of the bacteriophage are not naturally occurring. The composition can be substantially free of bacterial components such as bacterial endotoxin, bacterial host protein, and the like.

In an aspect, provided herein are bacteriophage having a genetic polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8, and bacteriophage or mutants having at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% 99%, 99.9, or 99.99% identity to any one of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In some embodiments, one or more of the bacteriophage are not naturally occurring.

In another aspect, there is provided a bacteriophage composition for use as a medicament, or for use in the treatment of a bacterial illness associated with P. aeruginosa. Any of the compositions provided herein are contemplated for use in the treatment of a disease or illness such as a P. aeruginosa infection. In some embodiments, one or more of the bacteriophage are not naturally occurring. In some embodiments, the compositions can include at least one naturally occurring bacteriophage. Corresponding methods of treating a disease comprising administration of the bacteriophage composition to a subject are also provided. The compositions according to this paragraph can include for example one more bacteriophage as described herein, including one or more of Pa223, Pa222, Pa193, Pa204, Pa197, Pa224, Pa226, Pa225 and Pa167. The compositions according to this paragraph can include for example one more bacteriophage comprising a nucleic acid having 76%-100% nucleic acid sequence identity to one or more of SEQ ID NOs: 1-8. In some embodiments, one or more of the bacteriophage are not naturally occurring.

In an aspect, provided herein are methods of use of bacteriophage compositions for treating a Pseudomonas infection. The Pseudomonas infection may be a Pseudomonas aeruginosa infection. The Pseudomonas infection may an infection that causes a sinus infection, nasal infection, respiratory infection, lung infection, urinary tract infection, intra-abdominal infection, septicemia, and/or bacteremia. The Pseudomonas infection may an infection that causes lung infection. The bacteriophage and compositions described in the preceding paragraphs or elsewhere herein can be used in the above-described methods. In some embodiments, one or more of the bacteriophage are not naturally occurring.

In an aspect, provided herein are methods of use of the composition in the manufacture of a medicament for treating a bacterial infection. The Pseudomonas infection may be a Pseudomonas aeruginosa infection. The Pseudomonas infection may an infection that causes a sinus infection, nasal infection, respiratory infection, lung infection, urinary tract infection, intra-abdominal infection, or bacteremia. The Pseudomonas infection may an infection that causes rhinosinusitis. The bacteriophage and compositions described in the above paragraphs or elsewhere herein can be used in the above-described methods and uses. In some embodiments, one or more of the bacteriophage are not naturally occurring.

In an aspect, provided herein are methods of treating a human with a bacterial infection including administration to the human a composition including one or more distinct bacteriophages that infect and lyse Pseudomonas aeruginosa bacteria. At least one of the bacteriophage can have a nucleic acid or a genome, that can include a polynucleotide sequence selected from one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a sequence having at least 76% identity to any one of SEQ ID NOS: 1-8. In some aspects one or more of the bacteriophage are not naturally occurring. The methods can include selecting a patient with a P. aeruginosa infection. The patient can be one that is resistant to an antibiotic treatment for P. aeruginosa. In some embodiments, one or more of the bacteriophage are not naturally occurring.

In an aspect, provided herein are methods of treating a bacterial infection with a confirmed P. aeruginosa infection and administration to the human of a composition including one or more distinct bacteriophages that infect and lyse Pseudomonas aeruginosa. At least one of the bacteriophage can include a nucleic acid comprising a nucleotide sequence selected from one or more of SEQ ID NO: 1 (Pa193, deposited under ECACC reference no. 17062004), SEQ ID NO: 2 (Pa204, deposited under ECACC reference no. 17062006), SEQ ID NO: 3 (Pa222, deposited under ECACC reference no. 17062003), SEQ ID NO: 4 (Pa223, deposited under ECACC reference no. 17062002), SEQ ID NO: 5, SEQ ID NO: 6 SEQ ID NO: 7, SEQ ID NO: 8, or a sequence having at least 76% identity to any one of SEQ ID NOS: 1-8. In some aspect's, the composition's target bacteria range is broader than the range of any individual bacteriophage in the composition or the sum of the individual bacteriophage in the composition. In some embodiments, one or more of the bacteriophage are not naturally occurring. The composition can be substantially free of bacterial components such as bacterial endotoxin, bacterial host protein, and the like.

In an aspect, provided herein are methods of modifying the microbial flora in a human including administering to said human a composition including one or more distinct bacteriophages having lytic activity against Pseudomonas aeruginosa. The one or more distinct bacteriophages are selected from bacteriophages having nucleic acid or a genome, including a nucleotide sequence of any one of SEQ ID NO: 1, SEQ ID NO:2, SEQ ID NO: 3, SEQ ID NO: 4, SEQ ID NO: 5, SEQ ID NO: 6, SEQ ID NO: 7, SEQ ID NO: 8, or a sequence having at least 76% identity to any one of SEQ ID NOS: 1-8. In some aspects one or more of the bacteriophage are not naturally occurring. The methods can include selecting a patient with a P. aeruginosa infection.

A further aspect relates to, for example, a bacteriophage composition for use in treating a bacterial infection in a subject, wherein the bacteriophage composition is administered to the subject, and wherein the bacterial infection includes Pseudomonas (e.g., Pseudomonas aeruginosa) infections. The bacteriophage and compositions described in the above paragraphs or elsewhere herein can be used in the above-described treatment methods and uses. In some embodiments, one or more of the bacteriophage are not naturally occurring.

Some aspects relate to uses of any composition described herein in the treatment of a Pseudomonas aeruginosa infection in a human. The uses can include, for example, administering a composition of one or more distinct bacteriophages to said human, wherein at least one of said one or more bacteriophage comprises a nucleic acid sequence comprising a polynucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and a sequence having at least 76% identity to any one of SEQ ID NOS: 1-8. In some uses, the one more of the bacteriophage are not naturally occurring.

Some aspects relate to uses of any composition described herein in the treatment of a confirmed non-pulmonary Pseudomonas aeruginosa infection in a human. The uses can include the treatment comprising administration to said human of a composition, the composition comprising at least two bacteriophages comprising a nucleic acid or a genome that includes a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10 and a sequence having at least 76% identity to any of SEQ ID NOS: 1-8, wherein the infection is not a pulmonary Pseudomonas aeruginosa infection. In some uses, the one more of the bacteriophage are not naturally occurring.

In an aspect, provided herein is a kit that includes a bacteriophage composition and instructions for use of the same.

### BRIEF DESCRIPTION OF THE DRAWINGS

Embodiments will now be described, by way of example only, with reference to the following Figures and the Examples.

FIG. 1 shows the efficacy of various treatments in a murine model of Pseudomonas aeruginosa lung infection. Treatment with the four phage cocktail described herein had equivalent efficacy in reducing lung colony forming units (bacterial load) as meropenem antibiotic treatment. Values that were below the limit of detection were set at the limit of detection to avoid undue bias during statistical analysis.

FIG. 2 is a summary chart showing phage candidates' single-copy genome similarities are shown in (% nucleotide identity). Within each cell, values are percent identity across the total alignment. The percent of the alignment made up by gaps is reported separately in parentheses by these gaps are included in the total percent identity.

### DETAILED DESCRIPTION

It is to be understood that the present disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

The detailed description of the present disclosure is divided into various sections only for the reader's convenience and disclosure found in any section may be combined with that in another section. Unless defined otherwise, all technical and scientific terms used herein have the same meaning as commonly understood by one of ordinary skill in the art to which the present disclosure belongs.

### Definitions

It must be noted that as used herein and in the appended claims, the singular forms “a,” “an,” and “the” include plural referents unless the context clearly dictates otherwise. Thus, for example, reference to “a bacteriophage” includes a plurality of bacteriophage.

As used herein, the term “about” when used before a numerical designation, e.g., temperature, time, amount, concentration, and such other, including a range, indicates approximations which may vary by (+) or (−) 10%, 5%, or 1%.

When a range (e.g., dosage range) is listed herein, it is to be understood that the value may include any value or range within the recited range(s), including endpoints.

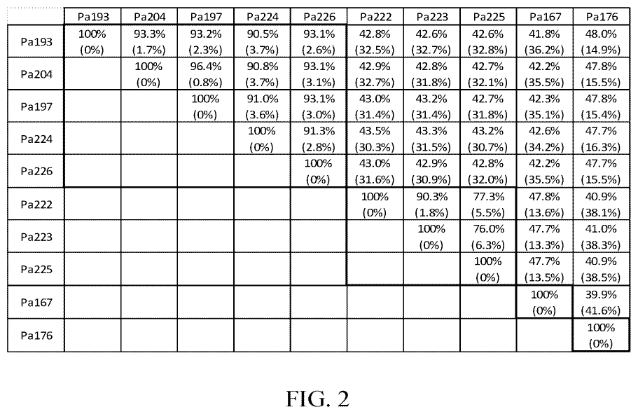

1) The term “mutant” or “variant” as used herein refers to a bacteriophage differing genetically from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006 by one or more nucleotide(s) but still retaining the ability to infect and lyse a Pseudomonas (e.g. P. aeruginosa) target bacteria. Mutants typically comprise e.g., silent mutations, conservative mutations, minor deletions, and/or minor replications of genetic material, and retain phenotypic characteristics of the reference bacteriophage. In an embodiment, the mutants retain any observable characteristic or property that is dependent upon the genome of the bacteriophage as described herein, i.e. phenotypic characteristics of said bacteriophage and/or lytic activity against Pseudomonas strains. Preferred mutants have mutants have less than 30% nucleic acid variation as compared to the genome of the reference bacteriophage, even more preferably less than 20%, more preferably less than 10%. Alternatively, or in combination, mutants have preferably less than 30% amino acid variation in a coded polypeptide sequence as compared to a polypeptide of the reference bacteriophage. Examples of phage that have less than 30% nucleic acid variation across the entire genome when compared to any one of Pa223 (SEQ ID NO: 4, deposited under ECACC reference no. 17062002), Pa222 (SEQ ID NO: 3, deposited under ECACC reference no. 17062003), Pa193 (SEQ ID NO: 1, deposited under ECACC reference no. 17062004) and Pa204 (SEQ ID NO: 2, deposited under ECACC reference no. 17062006 by one or more nucleotide(s) but still retaining the ability to infect and lyse a Pseudomonas (e.g. P. aeruginosa) target bacteria include Pa197 (SEQ ID NO: 5), Pa224 (SEQ ID NO: 6), Pa225 (SEQ ID NO: 7), and Pa226 (SEQ ID NO: 8) (See FIG. 2).

The term “% identity” or “% sequence identity” in relation to nucleic acid or amino acid sequences designates the level of identity or homology between said sequences and may be determined by techniques known in the art. Any of a variety of sequence alignment methods can be used to determine percent identity, including, without limitation, global methods, local methods and hybrid methods, such as segment approach methods. Protocols to determine percent identity are routine procedures within the scope of one skilled in the art. Global methods align sequences from the beginning to the end of the molecule and determine the best alignment by adding up scores of individual nucleotide pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement. Non-limiting methods include, e.g., BLAST, Match-box, see, e.g., Align-M, see, e.g., Ivo Van Walle et al., Align-M—A New Algorithm for Multiple Alignment of Highly Divergent Sequences, 20(9) Bioinformatics: 1428-1435 (2004). This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 100 nucleotides in length, preferably over a region that is 100-1000 or more nucleotides in length, or more preferably over a region that is substantially the entire genome. As noted throughout, some aspects relate to bacteriophage having sequence identity to specified strains and sequences described herein, to compositions of the same, and to methods of using the same. For example, some aspects herein relate to bacteriophage having 76%-100% sequence identity to a phage (e.g., Pa223, Pa222, Pa193, Pa204, Pa197, Pa224, Pa225, and Pa226) or sequence described herein (e.g., SEQ ID NOs. 10⁸). The identity can be at least about 76, 77%, 78%, 79%, 80%, 81%, 82%, 83%, 84%, 85%, 86%, 87%, 88%, 89%, 90%, 91%, 92%, 93%, 94%, 95%, 96%, 97%, 98% 99%, 99.9, or 99.99%. The bacteriophage variants can have the ability to infect and/or lyse Pseudomonas such as P. aeruginosa.

The term “complementation” as used herein refers to the ability of a bacteriophage with a particular genome to compensate for a different, distinct bacteriophage with a different genome. More specifically, bacteriophage insensitive mutants colonies (of target bacteria) may arise to a particular bacteriophage but may still be sensitive to a different bacteriophage. In other words, bacteriophage resistant mutant bacteria arising to one phage are still sensitive to another phage.

The term “generalized transduction” as used herein refers process by which any bacterial DNA may be transferred to another bacterium via a bacteriophage. It is a rare event: a very small percentage of phage particles happen to carry a donor bacterium's DNA, on the order of 1 phage in 10,000. In essence, this is the packaging of bacterial DNA into a viral envelope.

The term “treat” or “treating” as used herein encompasses prophylactic treatment (e.g. to prevent onset of a disease) as well as corrective treatment (treatment of a subject already suffering from a disease).

The term “lytic” or “lytic activity” designates the property of a bacteriophage to cause lysis of a bacterial cell. The lytic activity of a bacteriophage can be tested on a bacteria (e.g., S. aureus strains) according to techniques known in the art. The lytic cycle is named for the process that occurs when a phage has infected a cell, replicated new phage particles, and bursts through the host cell membrane. Some phage exhibit a lysogenic cycle during which the bacteriophage DNA remains practically dormant due to active repression of bacteriophage processes. Whenever the bacteria divides, the DNA of the phage is copied as well. In this way, the virus can continue replicating within its host without lysing the host. At a certain point, conditions may change and the phage enters a lytic cycle. “Obligately lytic” refers to phage that are unable to undergo a lysogenic cycle.

A use or method typically comprises administering a bacteriophage composition described herein to a subject. As used herein, a “subject” is a mammal, such as a human or other animal. In embodiments, the subject is a human.

The term “isolated” as used herein indicates that the bacteriophage are removed from its original environment in which it naturally occurs. In particular, an isolated bacteriophage is, e.g., cultivated, cultured separately from the environment in which it is naturally located. Some aspects herein relate to isolated bacteriophage (individually and/or collectively), compositions of the same, and methods of using the same to treat Pseudomonas infections. The isolated bacteriophage include, but are not limited to, those specifically described and listed herein, and sequence variants as described herein.

The term “purified” as used herein indicates that the bacteriophage are removed from manufacturing host bacteria. In particular, a purified bacteriophage has production impurities, such as bacterial components, removed from its manufacturing or production environment. Bacterial components include but are not limited to bacterial host proteins, lipids, and/or bacterial endotoxin. The term “purified” may also refer to genetic purification in which the strain of bacteriophage is genetically homogenous. Some aspects herein relate to purified bacteriophage (individually and/or collectively), compositions of the same, and methods of using the same to treat Pseudomonas infections. The purified bacteriophage include, but are not limited to, those specifically described and listed herein, and sequence variants as described herein.

As used herein, the term “substantially purified” refers to a composition containing less than 1%, less than 0.1%, less than 0.001%, or no detectable amount of contaminants such as host bacterial proteins or endotoxin. Also, as used herein, the term “substantially pure” when used to describe a bacteriophage strain refers to the genetic purity of the composition such that the strain is greater than 99%, greater than 99.9%, greater than 99.999%, or 100% of one particular nucleic acid sequence (e.g., a genomic sequence for example). Some aspects herein relate to substantially purified bacteriophage (individually and/or collectively), compositions of the same, and methods of using the same to treat Pseudomonas infections. The substantially purified bacteriophage include, but are not limited to, those specifically described and listed herein, and sequence variants as described herein.

Typically, a composition is substantially pure when at least 80%, more preferably at least 85%, more preferably at least 90%, more preferably at least 95%, more preferably at least 98%, and most preferably at least 99% of the total material (by volume, by wet or dry weight, or by mole percent or mole fraction) in a sample is free of impurities or genetic variants.

The term “substantially free” as used herein can refer to something having less than 10% of the substance that it is to be free from. For example, 0.01% to 10% free, including any subvalue and subrange therein. For example, 0.01, 0.02, 0.03, 0.04, 0.05, 0.06, 0.07, 0.08, 0.09, 1, 2, 3, 4, 5, 6, 7, 8, 9, or 10%.

The term “subject” or “patient” refers to a human or non-human animal. Preferably, the subject or patient is in need of treatment with the composition as described herein, e.g., has a bacterial infection susceptible to treatment with the composition.

A “synergistic amount” as used herein refers to the sum of a first amount (e.g., a bacteriophage) and a second amount (e.g., a different bacteriophage) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

Additional terms and phrases are defined below.

### Bacteriophage Compositions

In an aspect, provided herein are bacteriophage compositions including one or more obligately lytic bacteriophage that infect and lyse Pseudomonas selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), and mutants having a genomic sequence with at least 76% identity to one or more of SEQ ID NO: 1-8. In some cases, one or more or all of the individual bacteriophage is not prone to generalized transduction and/or does not carry antibiotic resistance genes. In some embodiments, the composition's target bacteria range is broader than the range of any individual bacteriophage in the composition or the sum of the individual bacteriophage in the composition. In some cases, each individual bacteriophage is not prone to generalized transduction and/or does not carry antibiotic resistance genes. In some embodiments, one or more of the bacteriophage are not naturally occurring. The composition can be substantially free of bacterial components such as bacterial endotoxin, bacterial host protein, and the like.

In embodiments, the mutants of Pa223, Pa222, Pa193, or Pa204 differ in genomic sequence by up to about 30%, up to about 20%, up to about 10%, up to about 9%, up to about 8%, up to about 7%, up to about 6%, up to about 5%, up to about 4%, up to about 3%, up to about 2% or up to about 1% compared to one or more of SEQ ID NO: 1-8. In one embodiment, the bacteriophage composition includes one or more additional bacteriophage. The bacteriophages disclosed herein and mutants thereof are useful for treating a bacterial infections, in particular a Pseudomonas infection.

The bacteriophages Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), were deposited at the European Collection of Cell Cultures (ECACC), Culture Collections, Public Health England, Porton Down, Salisbury, Wiltshire, SP4 0JG, United Kingdom, on 20 Jun. 2017. All of the deposits were made under the provisions of the Budapest Treaty on the International Recognition of the Deposit of Microorganisms for the Purposes of Patent Procedure.

In embodiments, a bacteriophage composition includes at least two bacteriophages selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), and mutants having at least 76% identity to a genomic sequence of Pa223, Pa222, Pa193, or Pa204. A bacteriophage composition including two or more bacteriophages may be referred to herein as a “panel” of bacteriophages.

In embodiments, a bacteriophage composition includes at least three bacteriophages selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), and mutants having at least 76% identity to a genomic sequence of Pa223, Pa222, Pa193, or Pa204.

In embodiments, a bacteriophage composition includes one bacteriophage selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), and mutants having at least 76% identity to a genomic sequence of Pa223, Pa222, Pa193, or Pa204. In embodiments, a bacteriophage composition includes two bacteriophages selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), and mutants having at least 76% identity to a genomic sequence of Pa223, Pa222, Pa193, or Pa204. In embodiments, a bacteriophage composition includes three bacteriophages selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), and mutants having at least 76% identity to a genomic sequence of Pa223, Pa222, Pa193, or Pa204. In embodiments, a bacteriophage composition includes four bacteriophages selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), and mutants having at least 76% identity to a genomic sequence of Pa223, Pa222, Pa193, or Pa204.

In embodiments, a bacteriophage composition includes each of Pa223 or mutant having at least 90% identity to a genomic sequence thereof, Pa222 or mutant having at least 90% identity to a genomic sequence thereof, Pa193 or mutant having at least 90% identity to a genomic sequence thereof, and Pa204 or mutant having at least 90% identity to a genomic sequence thereof.

In embodiments, the bacteriophage compositions include one or more (e.g. two or more) of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006). In embodiments, bacteriophage compositions include two or more of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006). In other embodiments, bacteriophage compositions include three or more of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006). In embodiments, bacteriophage compositions include Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006).

In embodiments, bacteriophage compositions consist essentially of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006), or mutants thereof. In an embodiment, a bacteriophage composition consists essentially of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006).

In embodiments, the only bacteriophage in the composition are Pa223 (deposited under ECACC reference no. 17062002), Pa222 (deposited under ECACC reference no. 17062003), Pa193 (deposited under ECACC reference no. 17062004) Pa204 (deposited under ECACC reference no. 17062006), and/or one or more mutants having at least 76% identity to a genomic sequence of Pa223, Pa222, Pa193, or Pa204. In embodiments, the only bacteriophage in the composition that target Pseudomonas are Pa223 (deposited under ECACC reference no. 17062002), Pa222 (deposited under ECACC reference no. 17062003), Pa193 (deposited under ECACC reference no. 17062004) Pa204 (deposited under ECACC reference no. 17062006), and/or one or more mutants having at least 76% identity to a genomic sequence of Pa223, Pa222, Pa193, or Pa204. In embodiments, the only bacteriophage in the composition that target P. aeruginosa are Pa223 (deposited under ECACC reference no. 17062002), Pa222 (deposited under ECACC reference no. 17062003), Pa193 (deposited under ECACC reference no. 17062004) Pa204 (deposited under ECACC reference no. 17062006), and/or one or more mutants having at least 76% identity to a genomic sequence of Pa223, Pa222, Pa193, or Pa204.

In an aspect, provided herein are bacteriophage compositions including one or more obligately lytic bacteriophage that infect and lyse Pseudomonas selected from Pa223 (SEQ ID NO: 4, deposited under ECACC reference no. 17062002), Pa222 (SEQ ID NO: 3, deposited under ECACC reference no. 17062003), Pa193 (SEQ ID NO: 1, deposited under ECACC reference no. 17062004) and Pa204 (SEQ ID NO: 2, deposited under ECACC reference no. 17062006). Each individual bacteriophage is not prone to generalized transduction and does not carry antibiotic resistance genes, and the composition is substantially free of bacterial components. The bacteriophage composition can be an alternative to conventional antibacterial agents/therapeutics, and overcomes one or more problems associated therewith. In some aspects the bacteriophage composition can be utilized as co-treatment or in combination with conventional antibacterial agents/therapeutics.

In embodiments, the bacteriophage compositions include one or more (e.g. two or more) of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006). In embodiments, bacteriophage compositions include two or more of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006). In other embodiments, bacteriophage compositions include three or more of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006). In embodiments, bacteriophage compositions include Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006).

In an aspect, provided herein are bacteriophage compositions including one or more obligately lytic bacteriophage that infect and lyse Pseudomonas, the bacteriophage having a polynucleotide sequence selected from SEQ ID NO: 1, SEQ ID NO: 2, SEQ ID NO: 3, SEQ ID NO: 4, and mutants having at least 76% to one or more of SEQ ID NO: 1-8. Each individual bacteriophage is not prone to generalized transduction and does not carry antibiotic resistance genes, and the composition is substantially free of bacterial components. The bacteriophage composition can be an alternative to conventional antibacterial agents/therapeutics, and overcomes one or more problems associated therewith. In some aspects the bacteriophage composition can be utilized as co-treatment or in combination with conventional antibacterial agents/therapeutics.

In embodiments, the bacteriophage composition includes at least two bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes at least three bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes one bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes two bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes three bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes four bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8.

In embodiments, the bacteriophage composition includes two or more bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes three or more bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes one bacteriophage having a polynucleotide sequence selected from SEQ ID NO: 1, SEQ ID NO: 2, SEQ ID NO: 3, SEQ ID NO: 4, and mutants having at least 76% identity thereto. In embodiments, the bacteriophage composition includes two bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes three bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes four bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8. In embodiments, the bacteriophage composition includes bacteriophage having a polynucleotide sequence selected from SEQ ID NO: 1, SEQ ID NO: 2, SEQ ID NO: 3, SEQ ID NO: 4, and mutants having at least 76% identity to one or more of SEQ ID NO: 1-8.

In an aspect, provided herein are bacteriophage compositions including one or more obligately lytic bacteriophage that infect and lyse Pseudomonas, the bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In some cases, each individual bacteriophage is not prone to generalized transduction and/or does not carry antibiotic resistance genes. In some embodiments, one or more of the bacteriophage are not naturally occurring. In some embodiments, the composition's target bacteria range is broader than the range of any individual bacteriophage in the composition or the sum of the individual bacteriophage in the composition. In some embodiments, one or more of the bacteriophage in the compositions are not naturally occurring.

In embodiments, the bacteriophage composition includes at least two bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition includes at least three bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition includes one bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition includes two bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition includes three bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition includes four bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

In embodiments, the bacteriophage composition includes two or more bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition includes three or more bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition one bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition two bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition three bacteriophage having a polynucleotide sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition four bacteriophage having a polynucleotide sequence selected from S SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In embodiments, the bacteriophage composition includes bacteriophage having a polynucleotide sequence selected from SEQ ID NO: 1, SEQ ID NO: 2, SEQ ID NO: 3, and SEQ ID NO: 4.

In an aspect, provided herein is a bacteriophage having a polynucleotide sequence selected from SEQ ID NO: 1, SEQ ID NO: 2, SEQ ID NO: 3, SEQ ID NO: 4, and mutants having at least 76% identity to any one of SEQ ID NO: 1-8.

In some embodiments, a bacteriophage composition is provided that consists essentially of a bacteriophage that includes the nucleic acid sequence of SEQ ID NO.: 1, a bacteriophage that includes the nucleic acid sequence of SEQ ID NO.: 2, a bacteriophage that includes the nucleic acid sequence of SEQ ID NO.: 3, and/or a bacteriophage that includes the nucleic acid sequence of SEQ ID NO.: 4, or mutants of each thereof. In embodiments, a bacteriophage composition consists essentially of a bacteriophage that includes the nucleic acid sequence of SEQ ID NO.: 1, a bacteriophage that includes the nucleic acid sequence of SEQ ID NO.: 2, a bacteriophage that includes the nucleic acid sequence of SEQ ID NO.: 3, and/or a bacteriophage that includes the nucleic acid sequence of SEQ ID NO.: 4, or mutants of each thereof, and a pharmaceutically acceptable excipient. In some embodiments, a bacteriophage composition is provided that consists essentially of bacteriophage that includes the nucleic acid sequence of SEQ ID NO.: 1, a bacteriophage that includes the nucleic acid sequence of SEQ ID NO.: 2, a bacteriophage that includes the nucleic acid sequence of SEQ ID NO.: 3, or a bacteriophage that includes the nucleic acid sequence of SEQ ID NO.: 4. In some embodiments, a bacteriophage composition is provided that consists essentially of two bacteriophage strains that include the nucleic acid sequence of SEQ ID NO.: 1, a bacteriophage that includes the nucleic acid sequence of SEQ ID NO.: 2, a bacteriophage that includes the nucleic acid sequence of SEQ ID NO.: 3, and/or a bacteriophage that includes the nucleic acid sequence of SEQ ID NO.: 4. In some embodiments, a bacteriophage composition is provided that consists essentially of three bacteriophage strains that includes the nucleic acid sequence of SEQ ID NO.: 1, a bacteriophage that includes the nucleic acid sequence of SEQ ID NO.: 2, a bacteriophage that includes the nucleic acid sequence of SEQ ID NO.: 3, and/or a bacteriophage that includes the nucleic acid sequence of SEQ ID NO.: 4. In some embodiments, a bacteriophage composition is provided that consists essentially of four bacteriophage strains that includes the nucleic acid sequence of SEQ ID NO.: 1, a bacteriophage that includes the nucleic acid sequence of SEQ ID NO.: 2, a bacteriophage that includes the nucleic acid sequence of SEQ ID NO.: 3, and/or a bacteriophage that includes the nucleic acid sequence of SEQ ID NO.: 4. The term “consists essentially of” as used in this context means that only the bacteriophage(s) explicitly indicated are present in the bacteriophage composition, but that said composition may also contain a further non-bacteriophage constituent, such as an appropriate carrier, diluent, conventional antibiotic or antibacterial agent, etc.

In some embodiments, a bacteriophage composition is provided that consists essentially of a bacteriophage that includes the nucleic acid sequence of SEQ ID NO.: 1, a bacteriophage that includes the nucleic acid sequence of SEQ ID NO.: 2, a bacteriophage that includes the nucleic acid sequence of SEQ ID NO.: 3, and a bacteriophage that includes the nucleic acid sequence of SEQ ID NO.: 4. In embodiments, a bacteriophage composition consists essentially of a bacteriophage that includes the nucleic acid sequence of SEQ ID NO.: 1, a bacteriophage that includes the nucleic acid sequence of SEQ ID NO.: 2, a bacteriophage that includes the nucleic acid sequence of SEQ ID NO.: 3, and a bacteriophage that includes the nucleic acid sequence of SEQ ID NO.: 4 and a pharmaceutically acceptable excipient.

The term “consists essentially of” as used in this context means that only the bacteriophage(s) explicitly indicated are present in the bacteriophage composition, but that said composition may also contain a further non-bacteriophage constituent, such as an appropriate carrier, diluent, conventional antibiotic or antibacterial agent, etc.

In one embodiment, the bacteriophage composition includes one or more additional bacteriophage. In a preferred embodiment, the one or more additional bacteriophage are suitable for treating a bacterial infection, in particular a Pseudomonas infection.

Representative genomic sequences of each of the bacteriophage are provided as follows: Pa223, deposited under ECACC reference no. 17062002 (SEQ ID NO.: 4), Pa222, deposited under ECACC reference no. 17062003 (SEQ ID NO.: 3), Pa193, deposited under ECACC reference no. 17062004 (SEQ ID NO.: 1), and Pa204, deposited under ECACC reference no. 17062006 (SEQ ID NO.: 2). A genomic sequence of a bacteriophage that differs from the sequence provided by up to about 30% is contemplated herein. Thus, the bacteriophage composition may include a bacteriophage having a nucleic acid sequence that differs from the sequence provided by up to about 20%, up to about 10%, up to about 9%, up to about 8%, up to about 7%, up to about 6%, up to about 5%, up to about 4%, up to about 3%, up to about 2%, or up to about 1%.

In an aspect, the bacteriophage composition includes at least one, at least two, at least three, or at least four bacteriophages such that the composition is effective against at least about 60% of target bacterial strains in a panel. For example, the bacteriophage composition is effective against (e.g., kills or lyses) at least 60% of Pseudomonas aeruginosa strains. In one embodiment, the bacteriophage composition includes at least one, at least two, at least three, or at least four bacteriophages such that the composition is effective against at least about 70% of target bacterial strains. In one embodiment, the bacteriophage composition includes at least one, at least two, at least three, or at least four bacteriophages such that the composition is effective against at least about 75% of target bacterial strains. In one embodiment, the bacteriophage composition includes at least one, at least two, at least three, or at least four bacteriophage such that the composition is effective against at least about 76% of target bacterial strains. In one embodiment, the bacteriophage composition includes at least one, at least two, at least three, or at least four bacteriophages such that the composition is effective against at least about 77% of target bacterial strains. In one embodiment, the bacteriophage composition comprises at least one, at least two, at least three, or at least four bacteriophages such that the composition is effective against at least about 78% of target bacterial strains. In one embodiment, the bacteriophage composition includes at least one, at least two, at least three, or at least four bacteriophages such that the composition is effective against at least about 79% of target bacterial strains. In one embodiment, the bacteriophage composition includes at least one, at least two, at least three, or at least four bacteriophages such that the composition is effective against at least about 80% of target bacterial strains. In one embodiment, the bacteriophage composition includes at least one, at least two, at least three, or at least four bacteriophages such that the composition is effective against at least about 81% of target bacterial strains. In one embodiment, the bacteriophage composition includes at least one, at least two, at least three, or at least four bacteriophages such that the composition is effective against at least about 82% of target bacterial strains. In one embodiment, the bacteriophage composition includes at least one, at least two, at least three, or at least four bacteriophage such that the composition is effective against at least about 83% of target bacterial strains. In one embodiment, the bacteriophage composition includes at least one, at least two, at least three, or at least four bacteriophages such that the composition is effective against at least about 84% of target bacterial strains. In one embodiment, the bacteriophage composition includes at least one, at least two, at least three, or at least four bacteriophage such that the composition is effective against at least about 85% of target bacterial strains. In one embodiment, the bacteriophage composition includes at least one, at least two, at least three, or at least four bacteriophage such that the composition is effective against at least about 90% of target bacterial strains. In another embodiment, the bacteriophage composition includes at least one, at least two, at least three, or at least four bacteriophages such that the composition is effective against one or more bacterial strains (or isolates) from a subject with a bacterial infection.

In embodiments, the range of target bacteria of the composition is broader than the range of target bacteria of any single bacteriophage included within the composition. Such activity can be considered synergistic as the effect of the composition (target killing range) is greater than the sum of individual effects (target killing range) of each component bacteriophage.

In embodiments where more than one bacteriophage is present in the bacteriophage composition, the composition is formulated such that each bacteriophage may be present at a ratio of between 1:10 and 10:1 (or any sub value or subrange there between including the endpoints) compared to the amount (e.g., concentration) of any other bacteriophage in the composition. In embodiments, each bacteriophage is present at a ratio of about 1:1 compared to one or more other bacteriophages in the composition. In embodiments, each bacteriophage is present at a ratio of about 1:2 compared to one or more other bacteriophages in the composition. In embodiments, each bacteriophage is present at a ratio of about 1:3 compared to one or more other bacteriophages in the composition. In embodiments, each bacteriophage is present at a ratio of about 1:4 compared to one or more other bacteriophages in the composition. In embodiments, each bacteriophage is present at a ratio of about 1:5 compared to one or more other bacteriophages in the composition. In embodiments, each bacteriophage is present at a ratio of about 1:6 compared to one or more other bacteriophages in the composition. In embodiments, each bacteriophage is present at a ratio of about 1:7 compared to one or more other bacteriophages in the composition. In embodiments, each bacteriophage is present at a ratio of about 1:8 compared to one or more other bacteriophages in the composition. In embodiments, each bacteriophage is present at a ratio of about 1:9 compared to one or more other bacteriophages in the composition. In embodiments, each bacteriophage is present at a ratio of about 1:10 compared to one or more other bacteriophages in the composition. In embodiments, each bacteriophage is present at a ratio of about 10:1 compared to one or more other bacteriophages in the composition. In embodiments, each bacteriophage is present at a ratio of about 5:1 compared to one or more other bacteriophages in the composition. In embodiments, each bacteriophage is present at a ratio of about 10:3 compared to one or more other bacteriophages in the composition. In embodiments, each bacteriophage is present at a ratio of about 5:2 compared to one or more other bacteriophages in the composition. In embodiments, each bacteriophage is present at a ratio of about 2:1 compared to one or more other bacteriophages in the composition. In embodiments, each bacteriophage is present at a ratio of about 5:3 compared to one or more other bacteriophages in the composition. In embodiments, each bacteriophage is present at a ratio of about 10:7 compared to one or more other bacteriophages in the composition. In embodiments, each bacteriophage is present at a ratio of about 5:4 compared to one or more other bacteriophages in the composition.

In one embodiment (alternatively or additionally) a “mutant” bacteriophage is capable of lysing the same target bacterial strains as Pa223 (deposited under ECACC reference no. 17062002), Pa222 (deposited under ECACC reference no. 17062003), Pa193 (deposited under ECACC reference no. 17062004) and/or Pa204 (deposited under ECACC reference no. 17062006), and optionally further capable of lysing one or more additional bacterial species or strains (preferably strains). In one embodiment a mutant may have at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% 99%, 99.9, or 99.99% sequence identity across its entire genome when compared to Pa223 (deposited under ECACC reference no. 17062002), Pa222 (deposited under ECACC reference no. 17062003), Pa193 (deposited under ECACC reference no. 17062004) and/or Pa204 (deposited under ECACC reference no. 17062006). In one embodiment a mutant may have at least about 90% to about 99% sequence identity across its entire genome when compared to Pa223 (deposited under ECACC reference no. 17062002), Pa222 (deposited under ECACC reference no. 17062003), Pa193 (deposited under ECACC reference no. 17062004) and/or Pa204 (deposited under ECACC reference no. 17062006), or any value or subrange there-between, including endpoints. Examples of phage that have less than 30% nucleic acid variation across the entire genome when compared to any one of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006 by one or more nucleotide(s) but still retaining the ability to infect and lyse a Pseudomonas (e.g. P. aeruginosa) target bacteria include Pa197 (SEQ ID NO: 5), Pa224 (SEQ ID NO: 6), Pa225 (SEQ ID NO: 7), and Pa226 (SEQ ID NO: 8).

For example, a Pa223 (deposited under ECACC reference no. 17062002) variant or mutant may have a nucleic acid, including genomic nucleic acids, including a polynucleotide sequence with 76%-100% sequence identify to SEQ ID NO: 4. For example, the variant or mutant may comprise a sequence having at least about 76%, 80%, 85%, 90%, 91%, 92%, 93%, 94%, 95%, 96%, 97%, 98%, or 99% sequence identity when compared to SEQ ID NO.: 4. As a further example, a Pa222 (deposited under ECACC reference no. 17062003) variant or mutant may have a nucleic acid, including a genome, comprising a polynucleotide sequence with 76%-100% sequence identify to SEQ ID NO: 4. For example, the variant or mutant may comprise a sequence having at least about 76%, 80%, 85%, 90%, 91%, 92%, 93%, 94%, 95%, 96%, 97%, 98%, or 99% sequence identity when compared to SEQ ID NO.: 3. Further, a Pa193 (deposited under ECACC reference no. 17062004) variant or mutant may have a nucleic acid, including a genome, that comprises a polynucleotide sequence with 76%-100% sequence identify to SEQ ID NO: 1. For example, the variant or mutant may comprise a sequence having at least about 76%, 80%, 85%, 90%, 91%, 92%, 93%, 94%, 95%, 96%, 97%, 98%, or 99% sequence identity when compared to SEQ ID NO.: 1. In some embodiments, a Pa204 (deposited under ECACC reference no. 17062006) variant or mutant may have a nucleic acid, including a genome, that comprises a polynucleotide sequence with 76%-100% sequence identify to SEQ ID NO: 1. For example, the variant or mutant may comprise a sequence having at least about 76%, 80%, 85%, 90%, 91%, 92%, 93%, 94%, 95%, 96%, 97%, 98%, or 99% sequence identity when compared to SEQ ID NO.: 2. The variants or mutants can be non-naturally occurring in some embodiments. The variants/mutants can be lytic against Pseudomonas.

In embodiments, a “mutant” may be a bacteriophage progeny. A bacteriophage progeny may be a bacteriophage obtainable after lysing Pseudomonas (e.g. P. aeruginosa) target bacteria using a bacteriophage as described herein (i.e. the “parent bacteriophage”). In other words, the bacteriophage progeny may be a second (or further) generation bacteriophage.

In embodiments, a bacteriophage progeny is obtainable by: contacting one or more bacteriophage(s) selected from Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006) with a Pseudomonas target bacteria such that the one or more bacteriophage(s) infects and lyses said target bacteria; and obtaining a bacteriophage released following lysis of said target bacteria. The bacteriophage progeny will typically comprise one or more nucleotide(s) mutation(s) when compared to the relevant parent bacteriophage. In embodiments, the bacteriophage progeny can have 76%-100% sequence identity (including any sub range or sub value therein, inclusive of endpoints) to a phage/sequence described herein. In embodiments, the bacteriophage progeny have a 76% identical genomic sequence when compared to the relevant parent bacteriophage. In embodiments, the bacteriophage progeny have a 80% identical genomic sequence when compared to the relevant parent bacteriophage. In embodiments, the bacteriophage progeny have a 90% identical genomic sequence when compared to the relevant parent bacteriophage. In embodiments, the bacteriophage progeny have a 91% identical genomic sequence when compared to the relevant parent bacteriophage. In embodiments, the bacteriophage progeny have a 92% identical genomic sequence when compared to the relevant parent bacteriophage. In embodiments, the bacteriophage progeny have a 93% identical genomic sequence when compared to the relevant parent bacteriophage. In embodiments, the bacteriophage progeny have a 94% identical genomic sequence when compared to the relevant parent bacteriophage. In embodiments, the bacteriophage progeny have a 95% identical genomic sequence when compared to the relevant parent bacteriophage. In embodiments, the bacteriophage progeny have a 96% identical genomic sequence when compared to the relevant parent bacteriophage. In embodiments, the bacteriophage progeny have a 97% identical genomic sequence when compared to the relevant parent bacteriophage. In embodiments, the bacteriophage progeny have a 98% identical genomic sequence when compared to the relevant parent bacteriophage. In embodiments, the bacteriophage progeny have a 99% identical genomic sequence when compared to the relevant parent bacteriophage. In embodiments, the bacteriophage progeny have 100% identical genomic sequence when compared to the relevant parent bacteriophage. The phage are genetically consistent throughout storage and production processes.

The term “obtainable” as used herein also encompasses the term “obtained.” In one embodiment the term “obtainable” means obtained.

The bacteriophage composition targets (and preferably kills) one or more Pseudomonas species or strain. In embodiments, the composition targets P. aeruginosa, including e.g. one or more strains of P. aeruginosa. In one embodiment, a Pseudomonas species or strain targeted by the bacteriophage is a Pseudomonas species or strain resistant to chemical antibiotics, such as a multi-drug resistant Pseudomonas species or strain.

In embodiments, the bacteriophage compositions described herein have been shown to be particularly effective at treating Pseudomonas species or strain infections resistant to chemical antibiotics, such as a multi-drug resistant Pseudomonas species or strain.

The bacteriophages may be provided in the form of a single therapeutic composition (preferred) or as a number of separate compositions each comprising one or more members of the composition. In embodiments where the bacteriophages are provided in a number of separate compositions, said bacteriophages may be administered to a subject sequentially or simultaneously (suitably simultaneously).

The bacteriophage may be propagated by any suitable method known in the art. For example, one or more bacteriophage(s) may be grown separately in host bacterial strains capable of supporting growth of the bacteriophage. Typically, the bacteriophage will be grown in said host bacterial strain to high concentrations, purified, titrated and combined to form the composition.

The amount of each bacteriophage employed (e.g. in a bacteriophage composition, method or use as described herein) may depend upon its virulence against the target bacterial isolate.

Count bacterial strains may be used in the development of a composition, i.e., bacterial strains which are indicators for individual prospective members of the composition (e.g. panel). A count strain may permit at least 1000 times more plaque formation by one prospective member of the bacteriophage composition than another. In this way, a composition (e.g. panel) that is consistently effective against a wide range of bacterial isolates may be achieved.

Typically, the one or more bacteriophage(s) may be combined to form a composition including at least about 1×10⁵, 1×10⁶, 1×10⁷, 1×10⁸, 1×10⁹ or 1×10¹⁰, or 1×10¹¹ plaque forming units (PFU) of each phage per ml of composition. The composition may include 1×10⁵ to 1×10¹¹ PFU of each phage per ml of composition. In embodiments, the composition may include 1×10⁵ to 1×10⁶ PFU, 1×10⁵ to 1×10⁷ PFU, 1×10⁵ to 1×10⁸ PFU, 1×10⁵ to 1×10⁹ PFU, or 1×10⁵ to 1×10¹⁰ PFU of each phage per ml of composition. In embodiments, the composition may include 1×10⁶ to 1×10⁷ PFU, 1×10⁶ to 1×10⁸ PFU, 1×10⁶ to 1×10⁹ PFU, 1×10⁶ to 1×10¹⁰ PFU, or 1×10⁶ to 1×10¹¹ PFU of each phage per ml of composition. In embodiments, the composition may include 1×10⁷ to 1×10⁸ PFU, 1×10⁷ to 1×10⁹ PFU, 1×10⁷ to 1×10¹⁰ PFU, or 1×10⁷ to 1×10¹¹ PFU of each phage per ml of composition. In embodiments, the composition may include 1×10⁸ to 1×10⁹ PFU, 1×10⁸ to 1×10¹⁰ PFU, or 1×10⁸ to 1×10¹¹ PFU of each phage per ml of composition. In embodiments, the composition may include 1×10⁹ to 1×10¹⁰ PFU or 1×10⁹ to 1×10¹¹ PFU of each phage per ml of composition. In embodiments, the composition may include 1×10¹⁰ to 1×10¹¹ PFU of each phage per ml of composition. In embodiments, one or more bacteriophage(s) may be combined to form a composition include 1×10⁵, 1×10⁶, 1×10⁷, 1×10⁸, 1×10⁹ or 1×10¹⁰, or 1×10¹¹ PFU of each phage per ml of composition. In some embodiments, the composition includes equal (or substantially equal) concentrations of each bacteriophage included herein. Suitable concentrations include any value or subrange within the indicated ranges, including endpoints.

In some aspects, the bacteriophage(s) in the composition are purified or substantially purified.

When selecting bacteriophages for inclusion in the composition, each of the selected phages was isolated from an environmental source and subsequently paired to a well-characterized P. aeruginosa strain that serves as its manufacturing host. After passaging and iterative selection, candidate phages were selected. The genomic sequences of these are not the same as the original, naturally-occurring sequence. Host-paired phages were purified to ensure that the resulting master stocks produced genetically and phenotypically consistent batches of each phage. After propagation, lysates were passed through a 0.2 μm filter to remove large cellular debris and subjected to a series of column-based purification steps to further remove host cell proteins and other bacterial debris and to replace growth medium.

In some embodiments, the bacteriophage composition includes at least one bacteriophage from the Myoviridae family and at least one bacteriophage from the Podoviridae family. In one embodiment, the bacteriophage composition includes at least two bacteriophage from the Myoviridae family and at least two bacteriophage from the Podoviridae family. In one embodiment, one or more of the bacteriophage is strictly lytic. Pa193 deposited under ECACC reference no. ECACC 17062004 and Pa204 deposited under ECACC reference no. ECACC 17062006 are of the Myoviridae family, and Pa222 deposited under ECACC reference no. ECACC 17062003 and Pa223 deposited under ECACC reference no. ECACC 17062002 are of the Podoviridae family. Preferably each bacteriophage is obligately lytic.

In some embodiments, the bacteriophage composition may further include one or more additional bacteriophages. The one or more additional bacteriophages may target a Staphylococcus aureus species or strain, additional strains of Pseudomonas aeruginosa, or a different bacterial target, for example selected from one or more of the following genera Staphylococcus, Helicobacter, Klebsiella, Listeria, Mycobacterium, Escherichia, Meningococcus, Campylobacter, Streptococcus, Enterococcus, Shigella, Pseudomonas (e.g. Pseudomonas species other than P. aeruginosa), Burkholderia, Clostridium, Legionella, Acinetobacter, Salmonella, or combinations thereof.

The one or more additional bacteriophage(s) may be one taught in WO 2009/044163 (incorporated herein by reference), including, but not limited to, a bacteriophage K and/or bacteriophage P68 described therein.

In one embodiment, the one or more additional bacteriophage(s) may be one or more taught in WO 2005/009451 A1, which is incorporated herein by reference. Suitably said one or more additional bacteriophage(s) may target Pseudomonas bacteria, such as Pseudomonas aeruginosa bacteria.

In one embodiment, the composition comprises one or more bacteriophage(s) taught in WO 2013/068743 A9 (incorporated herein by reference).

In some embodiments, the bacteriophage composition comprises one or more bacteriophage(s) as described herein and a preservative agent for storage. Storage may be refrigeration, freezer, ultra-freezer and the like. In one embodiment, the preservative agent is glycerol. In some embodiments, the preservative agent is present in the composition in an amount sufficient to preserve the composition, for example during storage in a refrigerator, freezer or ultra-freezer (e.g., at temperatures from about 0° C. to about −80° C., from about −20° C. to about −80° C., or about −80° C.), or in liquid nitrogen. In some embodiments, the preservative agent is present in the composition in an amount sufficient to preserve the composition during long-term storage, e.g., in a freezer, ultra freezer, or liquid nitrogen. In one embodiment, the preservative agent is between about 5% and about 50% glycerol; more preferably between about 10% and about 30% glycerol; most preferably about 20% glycerol. Suitable concentrations may be any value or subvalue within the recited ranges, including endpoints. Suitable concentrations may be any value or subvalue within the recited ranges, including endpoints.

### Formulations

In some embodiments, bacteriophage compositions provided herein further include a pharmaceutically acceptable carrier, diluent, excipient or combinations thereof. Suitable carriers, diluents and/or excipients may include isotonic saline solutions, such as phosphate-buffered saline. “Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

A bacteriophage composition as described herein may be formulated as a disinfectant composition. The disinfectant composition may be in the form of a spray or liquid wash for a surface. The composition may be a hand wash. Suitably where the composition is a formulation for topical application, it may take the form of a lotion, cream, ointment, paste, gel, foam, or any other physical form as a carrier generally known for topical administration. Such thickened topical formulations are particularly advantageous because the formulations adhere to the area of the skin on which the material is placed, thus allowing a localized high concentration of bacteriophages to be introduced to the particular area to be disinfected. For example, paraffin- and lanolin-based creams, which are particularly useful for the application of product to the nasal cavity, are generally known in the art. However, other thickeners, such as polymer thickeners, may be used. The formulations may also comprise one or more of the following: water, preservatives, active surfactants, emulsifiers, anti-oxidants, or solvents.

A bacteriophage composition as described herein may be formulated for nasal, oral, parenteral, intramuscular, intra-articular, intravenous, subcutaneous, transdermal, ocular or aural administration. Such a bacteriophage preparation may be used directly, refrigerated, lyophilized, stored frozen in aqueous or other solution with an appropriate cryoprotectant (e.g. 20% glycerol), freeze dried and rehydrated prior to use, or rendered stable in some other formulation including (but not limited to) tablet, emulsion, ointment, or impregnated wound dressing or other item. In embodiments, the bacteriophage composition includes saline (e.g., phosphate buffered saline, with or without magnesium). In embodiments, the bacteriophage composition includes a buffer. In embodiments, the buffer includes calcium salts or magnesium salts. In an embodiment, the mbuffer includes phosphate buffered saline and MgSO₄. The buffer may include 1 mM to 20 mM MgSO₄, 2 mM to 19 mM MgSO₄, 3 mM to 17 mM MgSO₄, 4 mM to 16 mM MgSO₄, 5 mM to 15 mM MgSO₄, 6 mM to 14 mM MgSO₄, 7 mM to 13 mM MgSO₄, 8 mM to 12 mM MgSO₄, 9 mM to 11 mM MgSO₄, or about 10 mM MgSO₄. The concentration may be any value or subrange within the recited ranges, including endpoints. For example, the buffer may include about 1 mM MgSO₄, about 2 mM MgSO₄, about 3 mM MgSO₄, about 4 mM MgSO₄, about 5 mM MgSO₄, about 6 mM MgSO₄, about 7 mM MgSO₄, about 8 mM MgSO₄, about 9 mM MgSO₄, about 10 mM MgSO₄, about 11 mM MgSO₄, about 12 mM MgSO₄, about 13 mM MgSO₄, about 14 mM MgSO₄, about 15 mM MgSO₄, about 16 mM MgSO₄, about 17 mM MgSO₄, about 18 mM MgSO₄, about 19 mM MgSO₄, or about 20 mM MgSO₄.

For embodiments directed to the treatment of a pulmonary bacterial infection, the bacteriophage composition may be formulated for pulmonary delivery via nasal or oral administration (e.g. by aerosolization or nebulization of the bacteriophage composition). Thus, in one embodiment the bacteriophage composition may be included in a nasal or pulmonary delivery means, such as a spray, a nebulizer, an inhaler or a respirator.

In one aspect, provided herein is a pulmonary delivery means (such as an inhaler, nebulizer, or a respirator) including the bacteriophage composition.

In embodiments, a bacteriophage composition described herein is formulated for nasal irrigation. Thus, a use or method of treatment described herein may include administering a bacteriophage composition to a subject by way of nasal irrigation.

In some embodiments, the bacteriophage composition is sterile. Such a sterile product may be suitable for parenteral administration in a subject.

In some embodiments, provided herein are aerosol formulations including the bacteriophage and/or bacteriophage compositions/formulations as described herein. Some embodiments relate to methods and uses of such aerosol formulations.

In embodiments, a bacteriophage composition described herein is formulated for intravenous (IV) administration. Intravenous administration may be by intravenous push or using an IV bag.

### Uses/Methods of Use

Provided herein is a use of a bacteriophage composition as a medicament (e.g., for treating a Pseudomonas infection). Corresponding methods of treating a disease comprising administration of the bacteriophage composition to a subject are also provided.

In an aspect, there is provided a bacteriophage composition for use in treating a bacterial infection. In related aspects, there is provided use of a bacteriophage composition in the manufacture of a medicament for treating a bacterial infection, as well as a method of treating a bacterial infection comprising administering the bacteriophage composition to a subject.

In an aspect, provided herein are methods of treating a bacterial infection including selecting a subject with a confirmed Pseudomonas infection and administering a bacteriophage composition as described herein.

The bacteriophage compositions are useful in treating a Pseudomonas (e.g. P. aeruginosa) bacterial infection. In one embodiment, the bacterial infection is a sinus, nasal or respiratory infection. In one embodiment, the bacterial infection is a lung infection. In one embodiment, the lung infection is in a patient with cystic fibrosis. In one embodiment, the bacterial infection is rhinosinusitis. In one embodiment, the bacterial infection is a urinary tract infection (or complicated urinary tract infection), intra-abdominal infection (or complicated intra-abdominal infection), septicemia (e.g., septicemia due to burns, uncontrolled bacteremia), or bacteremia (e.g., due to pneumonia, urinary tract infection, endocarditis, etc.). In one embodiment, the bacterial infection is an implant infection, such as a cardiac implant infection (e.g., ventricular assist device infection; pacemaker infection) or prosthetic joint infection. In one embodiment, the bacterial infection is endocarditis or prosthetic valve endocarditis. In one embodiment, the bacterial infection is pneumonia, such as hospital associated pneumonia, post-transplant pneumonia, or ventilator associated pneumonia. In one embodiment, the bacterial infection is a skin infection or skin structure infection. In some embodiments, the infection is characterized by the presence of a bacterial biofilm.

In embodiments, the bacterial infection may be chronic or acute.

In some embodiments, the subject has a bacterial infection that is not responding to one or more antibiotics. In some embodiments, the subject has a bacterial infection that fails to respond to standard-of-care antibiotics.

In embodiments, one or more bacterial isolates from the subject is tested for susceptibility to the bacterial composition prior to administration.

In some embodiments, the infection is an infection caused by an antibiotic resistant Pseudomonas species or strain, such as a multi-drug resistant Pseudomonas species or strain. For example and without limitation, the Pseudomonas species or strain may be resistant to fluoroquinolones, imipenem, gentamycin, amikacin, ciprofloxacin, ceftazidime, piperacillin or combinations thereof.

Provided herein is a bacteriophage composition for use in treating lung infection. In related aspects, there is provided use of a bacteriophage composition in the manufacture of a medicament for treating lung infection, as well as related methods of treatment of lung infection. The lung infection may be chronic or acute.

A use or method as described herein typically comprises administering a bacteriophage composition described herein to a subject. As used herein, a “subject” is a mammal, such as a human or other animal. Preferably, the term “subject” refers to a human subject. In one embodiment, the subject is a human subject with a Pseudomonas infection (e.g. a P. aeruginosa infection).

A bacteriophage composition may be administered to a subject in a therapeutically effective amount or a prophylactically effective amount.

As used herein, a “therapeutically effective amount” is any amount of the composition, which when administered alone or in combination to a subject for treating a bacterial infection (or a symptom thereof) is sufficient to effect such treatment of the infection, or symptom thereof.

As used herein, a “prophylactically effective amount” is any amount of the composition that, when administered alone or in combination to a subject inhibits or delays the onset or reoccurrence of a bacterial infection (or a symptom thereof). In some embodiments, the prophylactically effective amount prevents the onset or reoccurrence of a bacterial infection entirely. “Inhibiting” the onset means either lessening the likelihood of a bacterial infection's onset (or symptom thereof), or preventing the onset entirely.

An appropriate dosage range is one that produces the desired therapeutic effect (e.g., the composition is dosed in a therapeutically or prophylactically effective amount).

In embodiments, the composition includes 1×10⁵ to 1×10¹¹ PFU of each phage per ml of composition. In embodiments, the composition includes 1×10⁵ to 1×10⁶ PFU, 1×10⁵ to 1×10⁷ PFU, 1×10⁵ to 1×10⁸ PFU, 1×10⁵ to 1×10⁹ PFU, or 1×10⁵ to 1×10¹⁰ PFU of each phage per ml of composition. In embodiments, the composition includes 1×10⁶ to 1×10⁷ PFU, 1×10⁶ to 1×10⁸ PFU, 1×10⁶ to 1×10⁹ PFU, 1×10⁶ to 1×10¹⁰ PFU, or 1×10⁶ to 1×10¹¹ PFU of each phage per ml of composition. In embodiments, the composition includes 1×10⁷ to 1×10⁸ PFU, 1×10⁷ to 1×10⁹ PFU, 1×10⁷ to 1×10¹⁰ PFU, or 1×10⁷ to 1×10¹¹ PFU of each phage per ml of composition. In embodiments, the composition includes 1×10⁸ to 1×10⁹ PFU, 1×10⁸ to 1×10¹⁰ PFU, or 1×10⁸ to 1×10¹¹ PFU of each phage per ml of composition. In embodiments, the composition includes 1×10⁹ to 1×10¹⁰ PFU or 1×10⁹ to 1×10¹¹ PFU of each phage per ml of composition. In embodiments, the composition may include 1×10¹⁰ to 1×10¹¹ PFU of each phage per ml of composition. In embodiments, a bacteriophage composition is administered to a subject at a dosage of at least about 1×10⁵ PFU of each phage, at least about 1×10⁶ PFU of each phage, at least about 1×10⁷ PFU of each phage, at least about 1×10⁸ PFU of each phage, at least about 1×10⁹ PFU of each phage, at least about 1×10¹⁰ PFU of each phage, or at least about 1×10¹¹ PFU of each phage per ml of composition. In embodiments, one or more bacteriophage(s) may be combined to form a composition include 1×10⁵, 1×10⁶, 1×10⁷, 1×10⁸, 1×10⁹ or 1×10¹⁰, or 1×10¹¹ PFU of each phage per ml of composition. Dosages include any value or range within the recited ranges, including endpoints.

In some embodiments, bacteriophage compositions provided herein are administered to a subject at a dosage of at least about 1×10⁵ PFU total phage, at least about 1×10⁶ PFU total phage, at least about 1×10⁷ PFU total phage, at least about 1×10⁸ PFU total phage, at least about 1×10⁹ PFU total phage, at least about 1×10¹⁰ PFU total phage, or at least about 1×10¹¹ PFU of total phage. The bacteriophage composition is administered at a dosage of between about 1×10⁵ to about 1×10¹¹ PFU of total phage per ml of composition. In embodiments, the bacteriophage composition is administered at a dosage of between about 1×10⁵ to about 1×10⁶ PFU, between about 1×10⁵ to about 1×10⁷ PFU, between about 1×10⁵ to about 1×10⁸ PFU, between about 1×10⁵ to about 1×10⁹ PFU, or between about 1×10⁵ to about 1×10¹⁰ PFU of total phage per ml of composition. In embodiments, the bacteriophage composition is administered at a dosage of between about 1×10⁶ to about 1×10⁷ PFU, between about 1×10⁶ to about 1×10⁸ PFU, between about 1×10⁶ to about 1×10⁹ PFU, between about 1×10⁶ to about 1×10¹⁰ PFU, or between about 1×10⁶ to about 1×10¹¹ PFU of total phage per ml of composition. In embodiments, the bacteriophage composition is administered at a dosage of between about 1×10⁷ to about 1×10⁸ PFU, between about 1×10⁷ to about 1×10⁹ PFU, between about 1×10⁷ to about 1×10¹⁰ PFU, or between about 1×10⁷ to about 1×10¹¹ PFU of each phage per ml of composition. In embodiments, the bacteriophage composition is administered at a dosage of between about 1×10⁸ to about 1×10⁹ PFU, between about 1×10⁸ to about 1×10¹⁰ PFU, or between about 1×10⁸ to about 1×10¹¹ PFU of total phage per ml of composition. In embodiments, the bacteriophage composition is administered at a dosage of between about between about 1×10⁹ to about 1×10¹⁰ PFU, or between about 1×10⁹ to about 1×10¹¹ PFU of total phage per ml of composition. In embodiments, the bacteriophage composition is administered at a dosage of between about 1×10¹⁰ to about 1×10¹¹ PFU of total phage per ml of composition. A dosage may be 3×10⁹ PFU per milliliter composition. Dosages include any value or range within the recited ranges, including endpoints.

In some embodiments, the bacteriophage composition is administered at least once, twice, three times, or four times daily. Suitably the bacteriophage composition may be administered twice daily. In one embodiment, therefore, a dosage of at least about 1×10⁵ PFU of each phage is administered at least once, twice, three times, or four times daily. In one embodiment, therefore, a dosage of at least about 1×10⁶ PFU of each phage is administered at least once, twice, three times, or four times daily. In another embodiment at least about 1×10⁷ PFU of each phage is administered at least once, twice, three times, or four times daily. In a further embodiment at least about 1×10⁸ PFU of each phage is administered at least once, twice, three times, or four times daily. In another embodiment at least about 1×10⁹ PFU of each phage is administered at least once, twice, three times, or four times daily. In another embodiment at least about 1×10¹⁰ PFU of each phage is administered at least once, twice, three times, or four times daily. In another embodiment at least about 1×10¹¹ PFU of each phage is administered at least once, twice, three times, or four times daily. A dosage range between about 1×10⁵ PFU of each phage to about 1×10¹¹ PFU of each phage may be administered at least once, twice, three times, or four times daily. Preferably a dosage range between about 1×10⁷ PFU of each phage to about 1×10⁹ PFU of each phage may be administered at least once, twice, three times, or four times daily.

In some embodiments, the bacteriophage composition is administered every 2 hours, every 4 hours, every 6 hours, every 8 hours, every 12 hours, every 24 hours, every 48 hours, or every 72 hours. In some embodiments, the bacteriophage composition is administered every 2 hours. In some embodiments, the bacteriophage composition is administered every 4 hours. In some embodiments, the bacteriophage composition is administered every 6 hours. In some embodiments, the bacteriophage composition is administered every 8 hours In some embodiments, the bacteriophage composition is administered every 12 hours. In some embodiments, the bacteriophage composition is administered every 24 hours. In some embodiments, the bacteriophage composition is administered every 48 hours. In some embodiments, the bacteriophage composition is administered every 72 hours. Frequency of administration include any value or range within the recited ranges, including endpoints.

In some embodiments, the bacteriophage composition is administered for at least one day, at least 2 days, at least 3 days, at least 4 days, at least 5 days, at least 6 days, one week, at least two weeks, at least three weeks, at least four weeks, at least 5 weeks, at least 6 weeks, at least 7 weeks, at least 8 weeks, at least 9 weeks, at least 10 weeks, or more than 10 weeks. In some embodiments, the bacteriophage composition is administered for at least one day. In some embodiments, the bacteriophage composition is administered for at least one week. In some embodiments, the bacteriophage composition is administered for at least two weeks. In some embodiments, the bacteriophage composition is administered for at least three weeks. In some embodiments, the bacteriophage composition is administered for at least four weeks. In some embodiments, the bacteriophage composition is administered for at least five weeks. In some embodiments, the bacteriophage composition is administered for at least six weeks. In some embodiments, the bacteriophage composition is administered for between about 3 days and about 100 days. In an embodiment, the bacteriophage composition is administered for between about 3 days and about 60 days. In an embodiment, the bacteriophage composition is administered for between about 7 days and about 60 days. In an embodiment, the bacteriophage composition is administered for between about 14 days and about 60 days. In an embodiment, the bacteriophage composition is administered for between about 3 days and about 57 days. In an embodiment, the bacteriophage composition is administered for between about 7 days and about 57 days. In an embodiment, the bacteriophage composition is administered for between about 14 days and about 57 days. In an embodiment, the bacteriophage composition is administered for between about 14 days and about 30 days. In an embodiment, the bacteriophage composition is administered for greater than 3 days. In an embodiment, the bacteriophage composition is administered for greater than 7 days. In an embodiment, the bacteriophage composition is administered for at least 14 days. In an embodiment, the bacteriophage composition is administered for greater than 14 days. In an embodiment, the bacteriophage composition is administered for 56 days. In an embodiment, the bacteriophage composition is administered for 57 days. Duration of administration includes any value or range within the recited ranges, including endpoints.

A bacteriophage composition for use as a medicament may be administered by any route selected on the basis of the condition to be treated. In one embodiment the route of administration is nasal, oral, pulmonary, parenteral, intramuscular, intra-articular, intravenous, subcutaneous, transdermal, ocular, aural or combinations thereof. When used in the treatment of a pulmonary bacterial infection, the bacteriophage composition may be administered nasally or orally, for example via aerosolisation or nebulization using an appropriate pulmonary delivery means, such as an inhaler, nebulizer, or respirator. The composition may be administered to the patient via more than one route, for example intravenously and by inhalation, or intravenously and intra-articularly.

In one embodiment, an antibiotic (e.g. a chemical antibiotic) may be administered in combination with the bacteriophage composition. Combinatorial administration of antibiotics and bacteriophages is taught in WO 2008/110840 and WO 2005/009451, which teaching is incorporated herein by reference in its entirety. The antibiotic may be administered simultaneously or sequentially with the bacteriophage composition. One or more antibiotics may be administered after the composition such that bacteriophage replication has become established before antibiotic treatment begins. In this case, antibiotic treatment may be delayed for one or more hours or days from application of the one or more bacteriophages. In an embodiment, the antibiotic treatment may be delayed for at least 12 or 24 hours, suitably at least 48 hours. In another embodiment the antibiotic treatment may be delayed for at least 3 or 4 days, suitably at least 5 days. In another embodiment, the antibiotic treatment may be delayed for at least one week, for example at least 8, 9 or 10 days. Where a bacteriophage composition is administered to a subject, it will suffice that the composition as a whole is capable of targeting the bacterial infection, even if not all of the individual bacteriophages do so.

In some embodiments, a bacteriophage composition includes one or more antibiotics, such as one or more chemical antibiotics. A combination of a bacteriophage composition and an antibiotic (e.g. a chemical antibiotic) may provide an enhanced (e.g. synergistic) therapeutic showing unexpectedly improved efficacy when treating a Pseudomonas infection, particularly when used in treating lung infection.

An antibiotic may be selected based on sensitivity of a Pseudomonas species or strain to said antibiotic. The Pseudomonas species or strain may be the same species or strain present in a subject to be treated. In an embodiment, a Pseudomonas species or strain is taken from a subject to be treated and tested for antibiotic sensitivity. Sensitivity may be determined by in vitro sensitivity assays known in the art.

Alternatively or additionally, an antibiotic may be selected because it is known to be active against a bacteria known to be (or thought likely to be) present together with a Pseudomonas infection to be treated (e.g. as part of a bacterial biofilm).

In one embodiment, an antibiotic includes an antibiotic from one of the following antibiotic classes: fluoroquinolone, meropenem, carbepenem, aminoglycoside, cephalosporin, penicillins, beta lactamase inhibitors, monobatams, phosphonic acids, and polymixins. Non-limiting examples include imipenem, gentamycin, amikacin, ciprofloxacin, ceftazidime, piperacillin or combinations thereof, or pharmaceutically acceptable salts thereof.

Provided herein are uses or methods including administration of a bacteriophage composition to a subject in vivo; in vitro monitoring of the sensitivity of a sample of bacterial cells from an infection (e.g. present in the subject) or from another infection by the same strain to one or more antibiotic(s); and administration of said one or more antibiotic(s), when it has been established that said sensitivity to said one or more antibiotic(s) has been induced.

In an embodiment, provided herein is a method for restoring sensitivity to an antibiotic(s) by administering a composition as described herein. In embodiments. provided is a method for disrupting a biofilm by administering a composition as described herein. In an embodiment is provided a method for destroying a biofilm by administering a composition as described herein.

In an embodiment, the antibiotic (e.g. chemical antibiotic) is administered at a time period at which sensitivity of sampled bacteria to the antibiotic is induced by the composition. In some embodiments, the time period may be 6 hours, 12 hours, 24 hours, or 48 hours. In other embodiments, the bacteriophage composition and the antibiotic may be administered at intervals of one day to two months apart, preferably at intervals of one to four weeks apart, suitably at intervals of two weeks apart.

In one aspect, a bacteriophage composition may be used in a method of killing bacteria (e.g. Pseudomonas species or strains) on a surface. The method includes applying a bacteriophage composition (e.g. formulated as a disinfectant composition) to the surface. The surface is a site of contamination or prospective site of contamination.

In one embodiment, the surface is the skin of a mammal (e.g. a human), for example a nasal cavity. Alternatively or additionally, the surface may be equipment, medical equipment including but not limited to prostheses, implants, and the like, bedding, furniture, walls or floors (e.g. in a clinical environment). Alternatively, the surface may be that of a medical dressing or an implanted or implantable medical device (e.g. prosthetic joint or heart valve).

Provided herein are kits including a bacteriophage composition as described herein; and instructions for use of same (e.g. in medicine). The kit may further include an antibiotic (e.g. a chemical antibiotic) and optionally instructions for use of same in combination with the bacteriophage composition. The kit may also include means for testing antibiotic resistance. The kit may further include materials and/or equipment for administration of the bacteriophage composition.

In an embodiment, the instructions provide details for dosing a bacteriophage composition as described herein. In embodiments, the instructions included in the kit are for use of bacteriophage composition as described herein in treating a Pseudomonas infection, e.g. rhinosinusitis, lung infection, etc.

Provided herein is a use of a bacteriophage composition or kit for a non-medical application. For example, a bacteriophage composition or kit may be used in food hygiene, agriculture or crop protection, and/or in environmental hygiene applications. Thus, in one embodiment the kit includes instructions for use of a bacteriophage composition in a non-medical application.

In embodiments, provided herein is a bandage or wound dressing including a bacteriophage composition. The wound dressing may be a pad or sticking plaster-type dressing. The bacteriophages may be applied to the wound dressing or bandage as a disinfectant formulation or topical cream, prior to applying to the wound dressing or bandage. Alternatively, the wound dressing or bandage may be soaked in a carrier containing the bacteriophages and dried to impregnate said bacteriophages within the dressing or bandage. Bacteriophages may also be adsorbed onto the surface of the bandage or wound dressing using techniques generally known in the art. The advantage of this approach is that the bandage or wound dressing allows the bacteriophages to be brought into contact with a wound, which may contain the bacteria. In a related aspect are provided methods of inhibiting the growth of and/or treating and/or killing bacteria by applying a bandage or wound dressing to a subject.

Unless defined otherwise, all technical and scientific terms used herein have the same meaning as commonly understood by one of ordinary skill in the art to which this disclosure belongs.

This disclosure is not limited by the example methods and materials disclosed herein, and any methods and materials similar or equivalent to those described herein can be used in the practice or testing of embodiments of this disclosure. Numeric ranges are inclusive of the numbers defining the range. The headings provided herein are not limitations of the various aspects or embodiments of this disclosure.

Other definitions of terms may appear throughout the specification. Before the example embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a bacteriophage” includes a plurality of such candidate agents and reference to “the bacteriophages” includes reference to one or more bacteriophages and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### EXAMPLES

### Example 1: Development of Phage Cocktail

Experiments were conducted to create a bacteriophage therapy that met the following criteria: 1) Obligately lytic to avoid specialized transduction of bacterial genes; 2) Not known, by empirical testing and/or inference from genomics, to be prone to generalized transduction, and 3) Fully sequenced, to avoid phages with genes known to carry antibiotic resistance or bacterial virulence genes, and to help assess other lifestyle traits.

Collectively, the phages used together to treat a subject should: 1) Have broad activity against the target pathogen but no other species, to maximize potential utility and minimize off-target effects, and 2) Be capable of complementation, in which resistant mutants arising to one phage are sensitive to another phage.

In addition to characteristics of the phages themselves, material for clinical use should be produced in such a way as to give confidence that the final product retains these characteristics (i.e. are still the same phages) and does not contain potentially harmful (or harmful amounts) of impurities such as endotoxin or host cell proteins.

### Bacteriophage Isolation

Each of the selected phages was isolated from an environmental source and subsequently paired to a well-characterized Pseudomonas aeruginosa strain that serves as its manufacturing host. After passaging and iterative selection, candidate phages were selected. The genomic sequences of these are not the same as the original, naturally-occurring sequence Host-paired phages were purified to ensure that the resulting master stocks produced genetically and phenotypically consistent batches of each phage. Unless otherwise stated, all data is derived from the host-paired, plaque-purified phages. Phages were propagated in liquid culture using vegetable peptone media. Lysates were passed through a 0.2 μm filter to remove large cellular debris and, depending on the needs of subsequent testing, optionally subjected to a proprietary process of column-based purification steps to further remove host cell proteins and other bacterial debris and to replace growth medium with phosphate-buffered saline (PBS) containing 10 mM magnesium sulfate (PBS+Mg).

Stocks of four anti-P. aeruginosa bacteriophages (Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006) were developed. Pa193 deposited under ECACC no. 17062004 and Pa204 deposited under ECACC no. 17062006 are of the Myoviridae family, and Pa222 deposited under ECACC no. 17062003 and Pa223 deposited under ECACC no. 17062002 are of the Podoviridae family. All 4 phages have been characterized as strictly lytic by genome sequencing. Prior to each assay, the stock suspension of each bacteriophage was titrated against a selected P. aeruginosa bacterial strain using the soft agar overlay small drop assay, as described below. Equal concentrations of each bacteriophage were combined to form the bacteriophage cocktail (known as AB-PA01).

### Bacterial Strains and Growth Conditions

P. aeruginosa strains were isolated from endoscopically-guided sinus swabs, from patients who met the European Position Paper on Rhinosinusitis and Nasal Polyps (EPOS) 2012 criteria for chronic rhinosinusitis (CRS) (Fokkens et al., 2012, EPOS 2012: European position paper on rhinosinusitis and nasal polyps 2012. A summary for otorhinolaryngologists. Rhinology 50, 1-12). Clinical P. aeruginosa isolates of patients with CF were kindly donated by the Department of Otorhinolaryngology, Academic Medical Centre (Amsterdam, Netherlands) and P. aeruginosa sinus isolates from CRS patients with and without CF were kindly donated by the Department of Otolaryngology-Head and Neck Surgery, University of Alabama at Birmingham (Birmingham, AL). P. aeruginosa isolates were stored in 25% glycerol in nutrient broth at −80° C. P. aeruginosa laboratory reference strain ATCC 15692 (PAO1) was obtained from American Type Culture Collection (Manassas, VA, USA) as a control for phage sensitivity and biofilm assays. Isolates were plated from frozen glycerol stocks onto 1.5% nutrient agar, and broth cultures were grown in nutrient broth. Agar plates and broth cultures were incubated at 37° C.

### Minimum Inhibitory Concentration (MIC) Assays

Resistance to commonly used antibiotics was determined using broth microdilution minimum inhibitory concentration (MIC) assays, as described by Wiegand et al. (2008), Agar and broth dilution methods to determine the minimal inhibitory concentration (MIC) of antimicrobial substances. Nat. Protocols 3, 163-175. Antibiotics tested were: gentamicin, ciprofloxacin, ceftazidime, piperacillin, and amikacin, obtained from Sigma-Aldrich (Castle Hill, NSW, Australia). Isolates were designated as being sensitive, resistant, or having intermediate sensitivity to the antibiotics based on Clinical and Laboratory Standards Institute (CLSI) cut-offs.

### Example 2: Phage Activity and Bacterial Lineage

Phage activity was assessed using a modification of the small drop agar overlay method (Mazzocco et al. “Enumeration of bacteriophages using the small drop plaque assay system”. Methods Mol Biol. 2009; 501:81-5). Briefly, planktonic bacterial culture was mixed with molten dilute agar and poured evenly over an agar plate. When the top agar layer was set, serial dilutions of standardized phage solutions were spotted onto the overlay and plates incubated overnight at 37° C. Phage activity was indicated by clearing of the bacterial lawn at the site of phage application, and by the development of individual plaques as the phage sample is diluted. Strains were only considered sensitive if discrete plaques could be observed as the sample was diluted, indicating phage replication in addition to cell death. Lineage was determined by Public Health England using the 9-locus VNTR typing system described by Turton et al (Clin Microbiol Infect. 2010 August; 16(8):1111-6).

The combination of phages (AB-PA01) is active against P. aeruginosa strains having multiple genetic lineages and antibiotic resistance profiles, including clinically significant ones such as the Liverpool Epidemic Strain (LES) and the Manchester strain. Genetic lineage is not predictive of phage activity because the genes involved in lineage determination are not ones expected to be involved in phage activity. For example, four strains with identical VNTR profiles related to the Liverpool Epidemic Strain (SPS #1155, SPS #1156, SPS #1158, SPS #1168) were all sensitive to AB-PA01, but to different combinations of the phages. Results are summarized in Table 1. In the right-most columns, the black dots indicate that productive phage infection by that phage occurred.

TABLE 1
Phage activity against P. aeruginosa strains

| | | No. repeats at indicated minisatellite locus¹ | | | | | | | | | Productive Phage Infection | | | |
|---|---|---|---|---|---|---|---|---|---|---|---|---|---|---|
| Strain | Lineage | 172 | 211 | 213 | 214 | 217 | 222 | 207 | 209 | (61) | Pa193 | Pa204 | Pa222 | Pa223 |
| SPS1155 | Liverpool | 11 | 7 | 5 | 4 | 4 | 3 | 5 | 2 | (7) | | • | | |
| SPS1156 | | | | | | | | | | | • | | | |
| SPS1158 | | | | | | | | | | | • | • | | |
| SPS1168 | | | | | | | | | | | | | • | • |
| SPS1165 | | 11 | 7 | — | 4 | 4 | 3 | 5 | 2 | (7) | • | • | • | • |
| SPS1163 | | 11 | — | 5 | 4 | 4 | 3 | 5 | 2 | (8) | | • | | |
| SPS1164 | | | | | | | | | | (10) | | | • | • |
| SPS1159 | Manchester | 8 | 2 | 3 | — | 3 | 2 | 5 | 3 | (8) | • | • | | • |
| SPS1162 | | 9 | 2 | 3 | — | 3 | 2 | 5 | 3 | (5) | • | • | | |
| SPS1170 | | | | | | | | | | (9) | | • | | |
| SPS1173 | | | | | | | | | | (9) | • | | • | • |
| SPS1175 | | | | | | | | | | (8) | • | | • | • |
| SPS1176 | | | | | | | | | | (9) | | | | |

¹Each column gives the number of repeats present at the indicated minisatellite locus; a hyphen indicates no PCR product was amplified from that locus. The ninth locus, in parentheses, sometimes aids discrimination within types.

Sensitivity of seven P. aeruginosa strains was tested with four bacteriophage (Pa197 (SEQ ID NO: 5), Pa224 (SEQ ID NO: 6), Pa225 (SEQ ID NO: 7), and Pa226 (SEQ ID NO: 8)) that that have less than 10% nucleic acid variation across the entire genome when compared to any one of Pa223 (deposited under ECACC reference no. 17062002), Pa222 (deposited under ECACC reference no. 17062003), Pa193 (deposited under ECACC reference no. 17062004) and Pa204 (deposited under ECACC reference no. 17062006 by one or more nucleotide(s) but still retaining the ability to infect and lyse a Pseudomonas (e.g. P. aeruginosa) target bacteria. The phage showed lytic activity to at least one P. aeruginosa isolate. The data is shown in Table 2.

TABLE 2
Phage activity of variant bacteriophage against P. aeruginosa strains

| | Sensitivity (S) or Resistance (R) of Seven P. aeruginosa Isolates | | | | | | |
|---|---|---|---|---|---|---|---|
| Phage | SPS#1146 | SPS#1147 | SPS#1148 | SPS#1149 | SPS#1150 | SPS#1151 | SPS#1152 |
| Pa197 | S | R | R | S | S | S | R |
| Pa224 | R | R | R | S | S | S | S |
| Pa225 | S | S | S | S | R | S | S |
| Pa226 | R | R | R | S | R | S | S |

### Example 3: Bacterial Cell Surface Receptors for Phages

Strain origins for PAO1 mutants are described in Danis-Wlodarczyk et al (Sci Rep. 2016 Jun. 15; 6:28115). Testing for susceptibility of bacterial strains to each phage was done using the small drop method as described above.

To further analyze the breadth and features of target bacteria of the bacteriophage composition and the individual phage components, bacterial strains mutated in various cell receptors were tested for sensitivity to the bacteriophage. All four phages infected the Pseudomonas aeruginosa mutants lacking flagella and type IV pili, suggesting they do not rely on them for infection. Pa193 and Pa204 required B-band O-antigen to infect PAO1. Pa222 and Pa223 required intact LPS core and an additional factor that was present in the ΔwaaL strain but not the ΔwbpL strain. Pa222 and Pa223 produced notably larger, clearer plaques on strains with substantially reduced motility (ΔwaaL and ΔfliC). Results are summarized in Table 3. Despite some similarities in receptor usage, each phage infects different sets of bacterial strains and has shown the ability to complement resistance that develops to another phage (complementation data shown in Table 4).

TABLE 3
Phage activity against P. aeruginosa mutant strains

| | | Spot Score and Mean Apparent | | | |
|---|---|---|---|---|---|
| Strain | | Titer (PFU/mL) ² | | | |
| Designation | Description ¹ | Pa193 | Pa204 | Pa222 | Pa223 |
| Pa1116 | Standard Quantification Host | 1.2E+09 | 7.5E+08 | NA | NA |
| Pa1430 | Standard Quantification Host | NA | NA | 6.2E+08 | 6.1E+08 |
| PAO1 | In-house PAO1 Control | 9.7E+08 | 7.5E+07 | 5.5E+08 | 5.3E+08 |
| Δrmd (A−, B+) | Deficient in D-rhamnose | 1.2E+09 | 2.1E+08 | 4.5E+08 | 4.0E+08 |
| | biosynthesis; lack of A-band LPS | | | | |
| ΔrmLC (A−, B−, | Deficient in L-rhamnose | 0 | 0 | 0 | 0 |
| core-) | biosynthesis; truncated core, lacks | | | | |
| | A- and B-band LPS | | | | |
| ΔwaaL (A−, B−) | Lacks Waal (ligating O-polymer to | 0 | 0 | 5.7E+08 | 7.3E+08 |
| | core-lipid A); LPS is devoid of A- | | | | |
| | and B-band | | | | |
| ΔwbpL (A−, B−) | Lacks glucosyltransferase | 0 | 0 | 0 | 0 |
| | essential for initiation of both A- | | | | |
| | and B-band synthesis | | | | |
| ΔfliC ΔalgC ΔpilA | Lacks flagella, type IV pili, AlgC (A- | 0 | 0 | 0 | 0 |
| | band, core oligosaccharide, | | | | |
| | alginate synthesis) | | | | |
| ΔfliC ΔpilA | Lacks flagella and type IV pili | 1.4E+09 | 3.3E+08 | 8.4E+08 | 9.0E+08 |
| ΔfliC | Lacks flagella | 1.2E+09 | 3.3E+08 | 7.5E+08 | 7.0E+08 |
| WT | Parental strain for ΔfliC ΔalgC | 1.1E+09 | 3.9E+08 | 1.0E+08 | 1.0E+08 |
| | ΔpilA strains | | | | |

¹ Strain origins for PAO1 mutants are described in Danis-Wlodarczyk et al (2016)
² Titers are the mean of three replicates except where underlined values indicates that discrete plaques were only observed in one or two of the three replicates.

### Example 4: Resistance

An important feature of a bacteriophage therapy is that the phage should not create evolutionary pressure for the development of bacteria resistant to the phage. The frequency of spontaneous resistance to AB-PA01 was tested using a P. aeruginosa strain sensitive to all four component phages using a modification of O'Flynn et al (2004). Known quantities of phages and bacteria were combined in a molten dilute agar overlay and bacterial colonies counted after 24 or 48 h incubation at 37° C. Selected colonies were tested for phage susceptibility using the small drop method previously described.

The spontaneous frequency of P. aeruginosa resistance to AB-PA01 appeared to be lower than the frequencies of resistance to individual component phages, with fewer apparent bacteriophage-insensitive mutants (BIMs) being isolated when the four phages were used in combination. When apparent BIMs were isolated and directly tested for phage susceptibility, not all BIMs were truly resistant to the phage used to generate them (Table 4). The single BIM that grew in the presence of AB-PA01 was sensitive to Pa204, meaning no truly resistant bacteria colonies against AB-PA01 were generated. Cross-resistance between the two myoviruses or between the two podoviruses appeared to be more common than cross-resistance between a myovirus and a podovirus, but this was not absolute. The data show that the phage cocktail had unexpected resistance management properties.

TABLE 4
Summary of resistance data

| Phage | | | | | | |
|---|---|---|---|---|---|---|
| used to | | | | | | |
| generate | Bacterial | Sensitivity of Lawn to Indicated Phage | | | | |
| BIM | Lawn | Pa193 | Pa204 | Pa222 | Pa223 | AB-PA01 |
| None | parental | 4.0E+07 | 4.0E+07 | 4.0E+08 | 4.0E+08 | 4.0E+08 |
| Pa193 | BIM 1A | 0 | 1.4E+05 | 0 | 0 | 5.0E+04 |
| | BIM 1B | 0 | 1.0E+05 | 0 | 0 | 2.6E+04 |
| | BIM 2A | 0 | 1.0E+05 | 0 | 0 | 3.0E+04 |
| | BIM 2B | 0 | 0 | 2.0E+09 | 8.0E+06 | 2.0E+08 |
| | BIM 3A | 0 | 0 | 1.2E+09 | 5.0E+06 | 2.8E+08 |
| | BIM 3B | 4.0E+05 | 6.0E+06 | 8.0E+08 | 1.2E+07 | 2.8E+08 |
| Pa204 | BIM 1A | 0 | 0 | 2.0E+08 | 8.0E+06 | 4.0E+08 |
| | BIM 1B | 0 | 0 | 1.2E+09 | 1.2E+07 | 3.0E+08 |
| None | parental | 4.0E+07 | 4.0E+07 | 4.0E+08 | 4.0E+08 | 4.0E+08 |
| | BIM 2A | 0 | 1.6E+05 | 0 | 0 | 1.0E+05 |
| | BIM 2B | 0 | 1.0E+05 | 0 | 0 | 8.0E+03 |
| | BIM 3A | 0 | 0 | 8.0E+08 | 6.0E+06 | 2.6E+08 |
| | BIM 3B | 0 | 0 | 1.2E+09 | 6.0E+06 | 2.4E+08 |
| Pa222 | BIM 1A | 1.8E+08 | 1.6E+08 | 0 | 0 | 8.0E+07 |
| | BIM 1B | 1.4E+08 | 2.2E+08 | 0 | 0 | 1.0E+08 |
| | BIM 2A | 1.2E+08 | 2.2E+08 | 0 | 0 | 6.0E+07 |
| | BIM 2B | 0 | 1.0E+04 | 0 | 0 | 5.0E+03 |
| | BIM 2C | 2.0E+08 | 1.6E+08 | 0 | 0 | 1.2E+08 |
| | BIM 3A | 2.0E+08 | 2.0E+08 | 0 | 0 | 1.2E+08 |
| | BIM 3B | 2.0E+08 | 2.2E+08 | 0 | 0 | 2.2E+08 |
| Pa223 | BIM 1A | 1.6E+08 | 1.4E+08 | 0 | 0 | 8.0E+07 |
| | BIM 1B | 2.6E+08 | 1.4E+08 | 0 | 0 | 6.0E+07 |
| | BIM 2A | 2.0E+08 | 1.2E+08 | 6.0E+08 | 1.0E+07 | 3.0E+08 |
| | BIM 2B | 3.0E+08 | 2.4E+08 | 0 | 0 | 6.0E+07 |
| | BIM 3A | 2.2E+08 | 1.8E+08 | 0 | 0 | 8.0E+07 |
| | BIM 3B | 2.4E+08 | 2.0E+08 | 0 | 0 | 8.0E+07 |
| AB-PA01 | BIM 1 | 0 | 1.0E+05 | 0 | 0 | 1.0E+04 |

### Example 5: Synergy

Unexpectedly, a P. aeruginosa strain was identified on which the effect of AB-PA01 was fundamentally different from the effects of the individual component phages. When a lawn of this strain (AP1127) was created on semi-solid media, the combination of all four phages consistently yielded distinct plaques whereas no plaques were observed when the phages were tested individually. Plaques indicate that phage replication has occurred in the process of killing the cell. This indicates that the collective bactericidal effect of the 4-phage combination was greater than the simple additive effects of the individual phages (referred to as “synergy”). This result unexpectedly differs from the more common observation in which the combination of 4 phages yields plaques when one or more of the component phages also yields plaques, as is exemplified by the results from the manufacturing host and SPS #1721 summarized in Table 5.

TABLE 5
Synergy Results

| | Titer (PFU/mL) for each Phage-Bacteria Combination | | | | | AB-PA01 |
|---|---|---|---|---|---|---|
| P. aeruginosa Lawn | Pa193 | Pa204 | Pa222 | Pa223 | AB-PA01 | Result |
| Paired Manufacturing | 3.0E+08 | 7.4E+08 | 3.6E+08 | 1.4E+09 | 2.3E+08 | Expected |
| Host | | | | | | |
| SPS#1721 | 2.0E+06 | 0 | 4.0E+07 | 0 | 6.0E+06 | |
| AP1127 | 0 | 0 | 0 | 0 | 2.6E+03 | Unexpected |

### Example 6: Mouse Model of Acute P. aeruginosa Pneumonia

Based on the data above showing the efficacy of the bacteriophage cocktail in killing a variety of Pseudomonas aeruginosa strains, experiments investigating the efficacy of the cocktail in animas with bacterial infections were conducted. Immunocompetent CD-1 female mice were inoculated intranasally (IN) with clinical strain PA14, (6.26 log₁₀ CFU) in 50 μL of tryptic soy broth. At 2 and 6 hours post infection (hpi), 50 μL of phage mix AB-PA01 was administered IN to three dosage groups (n=5) consisting of 1.5×10⁷, 1.5×10⁸, or 1.5×10⁹ total PFU per dose. Meropenem (25 mg/kg) was administered subcutaneously at 2 and 6 hpi to a fourth group. A fifth group was infected, but treated with the phage diluent as a control. All mice were euthanized at 24 hours and CFU/lung pair determined. Blue values were below limit of detection and conservatively set at limit of detection. Statistics were performed

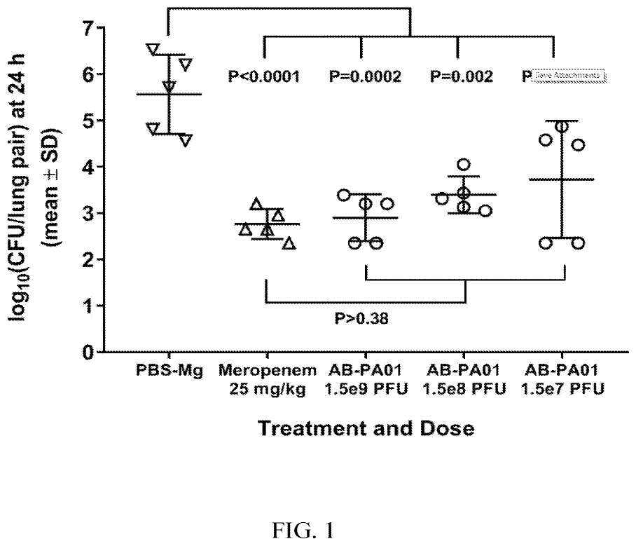

AB-PA01 efficacy was equivalent to meropenem in murine models of acute P. aeruginosa lung infection, causing a 2 to 3 log reduction in lung colony forming units (CFU) (FIG. 1). Values that were below the limit of detection were set at the limit of detection to avoid undue bias during statistical analysis.

### Example 7: Bacteriophage Administration to Human Patients

Bacteriophage cocktails were administered to human patients having P. aeruginosa infections that did not respond to antibiotic treatment alone. The patients were suffering from lung infection (post-transplant lung, patient was on immunosuppressant drugs), chronic lung infection (patient with cystic fibrosis), or ventilator-associated pneumonia.

A bacteriophage composition (“cocktail”) containing approximately equal ratios of four bacteriophage, ECACC 17062004 (Pa193), ECACC 17062006 (Pa204), ECACC 17062003 (Pa222), and ECACC 17062002 (Pa223), was administered to three of the patients having P. aeruginosa infections. The phage cocktail had approximately 80% coverage of P. aeruginosa strains, including multidrug-resistant isolates. Susceptibility of P. aeruginosa isolates from the patients was determined by soft agar overlay small drop assay prior to treatment. Patients were treated with 4×10⁹ PFU of the phage cocktail via intravenous administration every 6 hours or 12 hours, and/or via inhalation (nebulized) every 12 hours. Therapy was administered for 7 to 57 days, depending on indication. Patients were also administered the best available antibiotic therapy, as determined by the attending physician.

Treatment was well-tolerated and there were no treatment-related serious adverse events.

Throughout the duration of treatment, the bacterial isolates from the patients remained sensitive to the cocktail.

### Example 8: Additional Results from Bacteriophage Administration in Human Patients

Bacteriophage cocktails were administered to human patients having P. aeruginosa infections that did not respond to antibiotic treatment alone. The patients were suffering from pneumonia (cystic fibrosis, post-transplant, or ventilator-associated pneumonia), or bacteraemia due to burns.

A bacteriophage composition (“cocktail”) containing approximately equal ratios of four bacteriophage, ECACC 17062004 (Pa193), ECACC 17062006 (Pa204), ECACC 17062003 (Pa222), and ECACC 17062002 (Pa223), was administered to six patients having P. aeruginosa infections. The phage cocktail had approximately 80% coverage of P. aeruginosa strains, including multidrug-resistant isolates. Susceptibility of P. aeruginosa isolates from the patients was determined by the spot dilution method prior to treatment. Patients were treated with 4×10⁹ PFU of the phage cocktail via intravenous administration every 6 hours or 12 hours, and/or via inhalation (nebulized) every 12 hours. Therapy was administered for 7 to 57 days, depending on indication. Patients were also administered the best available antibiotic therapy, as determined by the attending physician.

Infections in the treated patients included pneumonia (cystic fibrosis, post-transplant, or ventilator-associated pneumonia), or bacteraemia due to burns.

Results showed 83% treatment success of patients in the modified intent-to-treat population (mITT, defined as all patients who meet criteria for the clinical diagnosis, whose bacterial isolate was susceptible to AB-PA01 and who received at least one dose of AB-PA01) treated with AB-PA01 at end of therapy. “Treatment success” was determined by the treating physician as a complete resolution or significant improvement of baseline signs and symptoms. One patient showed no improvement after treatment with AB-PA01 (determined by the treating physician as no resolution of baseline signs and symptoms, or death).

Over 600+ doses of AB-PA01 were administered, including 400+ doses intravenously, 90+ inhaled, 50+ topical, and 20+ oral. One patient discontinued treatment due to 2 adverse events. Treatment was well tolerated in the other 5 patients. There were no treatment-related SAEs.

All publications mentioned in the above specification are herein incorporated by reference. Various modifications and variations of the described methods and system as described herein will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### EMBODIMENTS

Embodiment 1-1. A bacteriophage composition comprising two or more strictly lytic bacteriophage from the Myoviridae family and two or more strictly lytic bacteriophage from the Podoviridae family.

Embodiment 1-2. The bacteriophage composition according to embodiment 1-1 comprising one or more bacteriophages selected from: ECACC 17062002, ECACC 17062003, ECACC 17062004, ECACC 17062006, and mutants thereof.

Embodiment 1-3. The bacteriophage composition according to embodiment 1-2 comprising at least two bacteriophages selected from: ECACC 17062002, ECACC 17062003, ECACC 17062004, ECACC 17062006, and mutants thereof.

Embodiment 1-4. The bacteriophage composition according to embodiment 1-2 comprising at least three bacteriophages selected from: ECACC 17062002, ECACC 17062003, ECACC 17062004, ECACC 17062006, and mutants thereof.

Embodiment 1-5. The bacteriophage composition according to any one of embodiments 1-1 to 1-4, comprising bacteriophages: ECACC 17062002, ECACC 17062003, ECACC 17062004, and ECACC 17062006, or mutants thereof.

Embodiment 1-6. The bacteriophage composition according to any one of the preceding embodiments consisting essentially of ECACC 17062002, ECACC 17062003, ECACC 17062004, and ECACC 17062006, or mutants thereof.

Embodiment 1-7. The bacteriophage composition according to any one of the preceding embodiments, wherein the mutant has at least 75% sequence identity across its entire genome when compared to ECACC 17062002, ECACC 17062003, ECACC 17062004, and/or ECACC 17062006.

Embodiment 1-8. The bacteriophage composition according to any one of the preceding embodiments, wherein the mutant has at least 80% sequence identity across its entire genome when compared to ECACC 17062002, ECACC 17062003, ECACC 17062004, and/or ECACC 17062006.

Embodiment 1-9. The bacteriophage composition according to any one of the preceding embodiments, wherein the mutant has at least 90% sequence identity across its entire genome when compared to ECACC 17062002, ECACC 17062003, ECACC 17062004, and/or ECACC 17062006.

Embodiment 1-10. The bacteriophage composition according to any one of the preceding embodiments, wherein the mutant has at least 95% sequence identity across its entire genome when compared to ECACC 17062002, ECACC 17062003, ECACC 17062004, and/or ECACC 17062006.

Embodiment 1-11. The bacteriophage composition according to embodiment 1-1 comprising one or more bacteriophages having a genomic sequence selected from: SEQ ID NO.: 1, SEQ ID NO.: 2, SEQ ID NO.: 3, SEQ ID NO.: 4, and mutants thereof.

Embodiment 1-12. The bacteriophage composition according to embodiment 1-11 comprising at least two bacteriophages having a genomic sequence selected from: SEQ ID NO.: 1, SEQ ID NO.: 2, SEQ ID NO.: 3, SEQ ID NO.: 4, and mutants thereof.

Embodiment 1-13. The bacteriophage composition according to embodiment 1-11 comprising at least three bacteriophages having a genomic sequence selected from: SEQ ID NO.: 1, SEQ ID NO.: 2, SEQ ID NO.: 3, SEQ ID NO.: 4, and mutants thereof.

Embodiment 1-14. The bacteriophage composition according to any one of embodiments 1-11 to 1-13, comprising bacteriophages having a genomic sequence of: SEQ ID NO.: 1, SEQ ID NO.: 2, SEQ ID NO.: 3, and SEQ ID NO.: 4, or mutants thereof.

Embodiment 1-15. The bacteriophage composition according to any one of embodiments 1-11 to 1-14, consisting essentially of bacteriophages having a genomic sequence of: SEQ ID NO.: 1, SEQ ID NO.: 2, SEQ ID NO.: 3, and SEQ ID NO.: 4, or mutants thereof.

Embodiment 1-16. The bacteriophage composition according to any one of embodiments 1-11 to 1-15, wherein the mutant has at least 75% sequence identity across its entire genome when compared to SEQ ID NO.: 1, SEQ ID NO.: 2, SEQ ID NO.: 3, and/or SEQ ID NO.: 4.

Embodiment 1-17. The bacteriophage composition according to any one of embodiments 1-11 to 1-15, wherein the mutant has at least 80% sequence identity across its entire genome when compared to SEQ ID NO.: 1, SEQ ID NO.: 2, SEQ ID NO.: 3, and/or SEQ ID NO.: 4.

Embodiment 1-18. The bacteriophage composition according to any one of embodiments 1-11 to 1-15, wherein the mutant has at least 90% sequence identity across its entire genome when compared to SEQ ID NO.: 1, SEQ ID NO.: 2, SEQ ID NO.: 3, and/or SEQ ID NO.: 4.

Embodiment 1-19. The bacteriophage composition according to any one of embodiments 1-11 to 1-15, wherein the mutant has at least 95% sequence identity across its entire genome when compared to SEQ ID NO.: 1, SEQ ID NO.: 2, SEQ ID NO.: 3, and/or SEQ ID NO.: 4.

Embodiment 1-20. The bacteriophage composition according to any one of the preceding embodiments further comprising an antibiotic (e.g. a chemical antibiotic).

Embodiment 1-21. The bacteriophage composition according to any one of the preceding embodiments further comprising a pharmaceutically acceptable carrier, diluent, excipient or combinations thereof.

Embodiment 1-22. The bacteriophage composition according to embodiment 1-21, wherein the pharmaceutically acceptable carrier, diluent, excipient or combinations thereof comprises MgSO₄.

Embodiment 1-23. The bacteriophage composition according to any one of the preceding embodiments, comprising about a 1:1 ratio of at least two of the bacteriophage strains.

Embodiment 1-24. The bacteriophage composition according to any one of the preceding embodiments, comprising at least about 1×10⁶ to about 1×10¹⁰ of at least one of the bacteriophage strains.

Embodiment 1-25. The bacteriophage composition according to any of the preceding embodiments comprising at least one cryprotectant.

Embodiment 1-26. The bacteriophage composition according embodiment 1-25, wherein the cryoprotectant comprises glycerol.

Embodiment 1-27. An aerosolized composition comprising a bacteriophage composition according to any of the preceding embodiments

Embodiment 1-28. A frozen composition comprising a bacteriophage composition according to any one of embodiments 1-1 to 1-26.

Embodiment 1-29. A composition according to any one of the preceding embodiments for use as a medicament.

Embodiment 1-30. A bacteriophage composition according to any one of the preceding embodiments for use in treating a bacterial infection.

Embodiment 1-31. Use of a bacteriophage composition according to any one of embodiments 1-1 to 1-26 in the manufacture of a medicament for treating a bacterial infection.

Embodiment 1-32. A method of treating a bacterial infection comprising administering the bacteriophage composition according to any one of embodiments 1-1 to 1-24 to a subject.

Embodiment 1-33. The bacteriophage composition for use, use, or method according to any one of embodiments 1-29 to 1-32, wherein the bacterial infection is a sinus, nasal or respiratory infection.

Embodiment 1-34. The bacteriophage composition for use, use, or method according to any one of embodiments 1-29 to 1-32, wherein the bacterial infection is a urinary tract infection, intra-abdominal infection, skin infection, skin structure infection, or bacteremia.

Embodiment 1-35. The bacteriophage composition for use, use, or method according to any one of embodiments 1-29 to 1-32, wherein the bacterial infection is a lung infection.

Embodiment 1-36. The bacteriophage composition for use, use, or method according to any one of embodiments 1-29 to 1-32, wherein the bacterial infection is endocarditis.

Embodiment 1-37. The bacteriophage composition for use, use, or method according to any one of embodiments 1-29 to 1-32, wherein the bacterial infection is an implant infection.

Embodiment 1-38. The bacteriophage composition for use, use, or method according to any one of embodiments 1-29 to 1-32, wherein the bacterial infection is associated with cystic fibrosis.

Embodiment 1-39. The bacteriophage composition for use, use, or method according to any one of embodiments 1-29 to 1-32, wherein the bacterial infection is resistant to antibiotics.

Embodiment 1-40. The bacteriophage composition for use, use, or method according to any one of embodiments 1-29 to 1-32, wherein the bacterial infection is a Pseudomonas bacterial infection.

Embodiment 1-41. The bacteriophage composition for use, use, or method according to any one of embodiments 1-29 to 1-32, wherein the bacterial infection is a Pseudomonas aeruginosa bacterial infection.

Embodiment 1-42. The bacteriophage composition for use, use, or method according to any one of embodiments 1-29 to 1-41, wherein the bacterial infection is an infection characterized by the presence of a bacterial biofilm.

Embodiment 1-43. A bacteriophage composition according to any one of embodiments 1-1 to 1-28 for use in treating rhinosinusitis.

Embodiment 1-44. Use of a bacteriophage composition according to any one of embodiments 1-1 to 1-28 in the manufacture of a medicament for treating rhinosinusitis.

Embodiment 1-45. A method of treating rhinosinusitis comprising administering the bacteriophage composition according to any one of embodiments 1-1 to 1-28 to a subject.

Embodiment 1-46. The bacteriophage composition for use, use, or method according to any one of embodiments 1-41 to 1-45, wherein the rhinosinusitis is chronic or acute rhinosinusitis.

Embodiment 1-47. The bacteriophage composition for use, use, or method according to any one of embodiments 1-41 to 1-46, wherein the rhinosinusitis is caused by a Pseudomonas bacterial infection.

Embodiment 1-48. The bacteriophage composition for use, use, or method according to any one of embodiments 1-41 to 1-47, wherein the rhinosinusitis is caused by a Pseudomonas aeruginosa bacterial infection.

Embodiment 1-49. The bacteriophage composition for use, use, or method according to any one of embodiments 1-41 to 1-48, wherein the rhinosinusitis is characterized by the presence of a bacterial biofilm.

Embodiment 1-50. The bacteriophage composition for use, use, or method according to any one of embodiments 1-29 to 1-49, further comprising administering an antibiotic (e.g. a chemical antibiotic) to the subject.

Embodiment 1-51. The bacteriophage composition for use, use, or method according to embodiment 1-50, wherein the antibiotic is a fluoroquinolone, carbepenem, aminoglycoside, cephlasporin, penicillin, beta lactam, or beta lactamase inhibitor.

Embodiment 1-52. The bacteriophage composition for use, use, or method according to any one of embodiments 1-29 to 1-51, wherein the composition is applied to the surface of the respiratory tract or lung tissue.

Embodiment 1-53. The bacteriophage composition for use, use, or method according to embodiment 1-52, wherein the application is done via an aerosolized formulation of the composition.

Embodiment 1-54. The bacteriophage composition for use, use, or method according to any one of embodiments 1-29 to 1-51, wherein the composition is administered intravenously.

Embodiment 1-55. A kit comprising: a. a bacteriophage composition according to any one of embodiments 1-1 to 1-28; and b. instructions for use of same (e.g. in medicine).

Embodiment 1-56. The kit according to embodiment 1-55 further comprising an antibiotic (e.g. a chemical antibiotic) and instructions for use of same in combination with the bacteriophage composition.

Embodiment 1-57. The kit according to embodiment 1-55 or 1-56, wherein said instructions are for use of same in treating an infection.

Embodiment 1-58. The kit according to embodiment 1-57, wherein said instructions are for use of same in treating a Pseudomonas aeruginosa infection.

Embodiment 1-59. The kit according to embodiment 1-57 or 1-58, wherein the infection is a sinus infection, nasal infection, respiratory infection, lung infection, urinary tract infection, intra-abdominal infection, or bacteremia.

Embodiment 1-60. A method of killing bacteria on a surface, said method comprising applying a bacteriophage composition according to any one of embodiments 1-1 to 1-28 to the surface.

Embodiment 1-61. The method according to embodiment 1-60, wherein the surface is the skin of a mammal, equipment (suitably medical equipment), bedding, furniture, walls, floors, or combinations thereof.

Embodiment 1-62. Use of the bacteriophage composition according to any one of embodiments 1-1 to 1-28 or the kit according to any one of embodiments 1-55 to 1-59 for a non-medical application.

Embodiment 1-63. A bandage or wound dressing comprising a bacteriophage composition according to any one of embodiments 1-1 to 1-28.

Embodiment 1-64. A method for manufacturing a bacteriophage composition, said method comprising admixing at least two bacteriophages selected from: ECACC 17062002, ECACC 17062003, ECACC 17062004, ECACC 17062006, and mutants thereof.

Embodiment 1-65. A method for manufacturing a bacteriophage composition, said method comprising admixing at least two bacteriophages having a genomic sequence selected from: SEQ ID NO.: 1, SEQ ID NO.: 2, SEQ ID NO.: 3, SEQ ID NO.: 4, and mutants thereof.

Embodiment 1-66. The method of embodiment 1-65, wherein the mutant has at least 75% sequence identity across its entire genome when compared to SEQ ID NO.: 1, SEQ ID NO.: 2, SEQ ID NO.: 3, and/or SEQ ID NO.: 4.

Embodiment 1-67. A bacteriophage composition obtainable by the method of any one of embodiments 1-64 to 1-66.

Embodiment 1-68. The composition, use, method, or kit of any one of the preceding embodiments, wherein the bacteriophage of the composition infect and lyse Pseudomonas.

Embodiment 1-69. The composition, use, method, or kit of any one of the preceding embodiments, wherein the bacteriophage of the composition infect and lyse Pseudomonas aeruginosa.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. A method of treating a bacterial infection comprising administering a composition comprising one or more obligately lytic bacteriophage that infect and lyse Pseudomonas, the bacteriophage selected from: bacteriophage having at least 99% identity to a genomic sequence of Pa222 (deposited under ECACC reference no. 17062003), bacteriophage having at least 97% identity to a genomic sequence of Pa193 (deposited under ECACC reference no. 17062004), or bacteriophage having at least 97% identity to a genomic sequence of Pa204 (deposited under ECACC reference no. 17062006), wherein the composition is substantially free of bacterial components, to a subject.

37. A method of treating a bacterial infection comprising:
a) selecting a patient with a confirmed Pseudomonas infection and,
b) administering a composition comprising one or more obligately lytic bacteriophage that infect and lyse Pseudomonas, the bacteriophage selected from: bacteriophage having at least 99% identity to a genomic sequence of Pa222 (deposited under ECACC reference no. 17062003), bacteriophage having at least 97% identity to a genomic sequence of Pa193 (deposited under ECACC reference no. 17062004), or bacteriophage having at least 97% identity to a genomic sequence of Pa204 (deposited under ECACC reference no. 17062006), wherein the composition is substantially free of bacterial components, to the subject.

38. The method of claim 37 wherein the composition comprises a total of 1×10⁵ to about 1×10¹¹ PFU.

39. The method of claim 37 wherein the composition is administered every 6 hours.

40. The method of claim 37 wherein the composition is administered every 12 hours.

41. The method of claim 37 wherein the composition is administered for at least 7 days.

42. The method according to claim 36, wherein the bacterial infection is a sinus, nasal, lung, respiratory infection.

43. The method according to claim 42, wherein the bacterial infection is a urinary tract infection, intra-abdominal infection, skin infection, skin structure infection, or bacteremia.

44. The method according to claim 42, wherein the bacterial infection is a lung infection.

45. The method according to claim 36, wherein the bacterial infection is endocarditis.

46. The method according to claim 36, wherein the bacterial infection is an implant infection.

47. The method according to claim 36, wherein the bacterial infection is associated with cystic fibrosis.

48. The method according to claim 36, wherein the bacterial infection is resistant to antibiotics.

49. The method according to claim 36, wherein the bacterial infection is a Pseudomonas bacterial infection.

50. The method according to 36, wherein the bacterial infection is a Pseudomonas aeruginosa bacterial infection.

51. (canceled)

52. (canceled)

53. A method of treating a lung infection comprising administering a composition comprising one or more obligately lytic bacteriophage that infect and lyse Pseudomonas, the bacteriophage selected from: bacteriophage having at least 99% identity to a genomic sequence of Pa222 (deposited under ECACC reference no. 17062003), bacteriophage having at least 97% identity to a genomic sequence of Pa193 (deposited under ECACC reference no. 17062004), or bacteriophage having at least 97% identity to a genomic sequence of Pa204 (deposited under ECACC reference no. 17062006), wherein the composition is substantially free of bacterial components, to a subject.

54. The method according to claim 53, wherein the lung infection is caused by a Pseudomonas bacterial infection.

55. The method according to claim 54, wherein the lung infection is caused by a Pseudomonas aeruginosa bacterial infection.

56. The method according to claim 53, further comprising administering an antibiotic and/or immunosuppressant to the subject.

57. The method according to claim 56, wherein the antibiotic is a fluoroquinolone, carbepenem, aminoglycoside, cephalosporin, penicillins, beta lactamase inhibitor, monobactams, phosphonic acids, or polymixins.

58. The method according to claim 53, wherein the composition is applied to the surface of the respiratory tract or lung tissue.

59. The method according to claim 58, wherein the application is done via an aerosolized formulation of the composition.

60. The method according to claim 53, wherein the composition is administered intravenously.

61. (canceled)

62. (canceled)

63. (canceled)

64. (canceled)

65. (canceled)

66. A method of killing bacteria on a surface, said method comprising applying a composition comprising one or more obligately lytic bacteriophage that infect and lyse Pseudomonas, the bacteriophage selected from: bacteriophage having at least 99% identity to a genomic sequence of Pa222 (deposited under ECACC reference no. 17062003), bacteriophage having at least 97% identity to a genomic sequence of Pa193 (deposited under ECACC reference no. 17062004), or bacteriophage having at least 97% identity to a genomic sequence of Pa204 (deposited under ECACC reference no. 17062006), wherein the composition is substantially free of bacterial components, to the surface.

67. The method according to claim 66, wherein the surface is the skin of a mammal, equipment, medical equipment, prostheses, implant, bedding, furniture, walls, floors, or combinations thereof.

68. (canceled)

69. (canceled)

70. A method for manufacturing a bacteriophage composition, said method comprising admixing at least two bacteriophages selected from: Pa222, Pa193, and Pa204, and bacteriophage having at least 90% identity thereto.

71. A method for manufacturing a bacteriophage composition, said method comprising admixing at least two bacteriophages having a genomic sequence selected from: SEQ ID NO: 1, SEQ ID NO: 2, SEQ ID NO:3, and bacteriophage having at least 90% identity thereto.

72. (canceled)

73. The method of claim 71, wherein the bacteriophage of the composition infect and lyse Pseudomonas.

74. The method of claim 71, wherein the bacteriophage of the composition infect and lyse Pseudomonas aeruginosa.
